(19) United States
(12) Patent Application Publication
(10) Pub. No.: US 2009/0227560 A1
(43) Pub. Date: Sep. 10, 2009

(54) Substituted imidazole compound and use thereof

(76) Inventors: Takanobu Kuroita, Osaka-shi (JP); Tsuneo Oda, Osaka-shi (JP); Yasutomi Asano, Osaka-shi (JP); Naohiro Taya, Osaka-shi (JP); Kouichi Iwanaga, Osaka-shi (JP); Hidekazu Tokuhara, Osaka-shi (JP); Yoshiyuki Fukase, Osaka-shi (JP)

Correspondence Address: WENDEROTH, LIND & PONACK, L.L.P., 2033 K STREET N. W., SUITE 800, WASHINGTON, DC, 20006-1021, US

(21) Appl. No.: 12/216,874

(22) Filed: Jul. 11, 2008

Related U.S. Application Data
(63) Continuation-in-part of application No. 12/081,917, filed on Apr. 23, 2008.

(30) Foreign Application Priority Data
Apr. 27, 2007 (JP) 120292/2007
Aug. 8, 2007 (JP) 207271/2007

(51) Int. Cl. A61K 31/496; C07D 403/06; C07D 403/14; C07D 413/14; A61K 31/5377; A61P 9/12

(52) U.S. Cl. 514/210.18; 544/370; 514/254.05; 544/105

(57) ABSTRACT

The present invention relates to a compound represented by the formula:

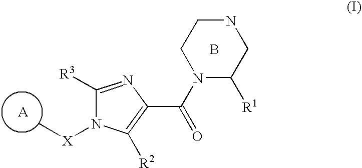

wherein each symbol is as defined in the description, or a salt thereof or a prodrug thereof.

The compound of the present invention has a superior renin inhibitory activity, and thus is useful as an agent for the prophylaxis or treatment of hypertension, various organ damages attributable to hypertension, and the like.

### TECHNICAL FIELD

The present invention relates to a substituted imidazole compound and the like, which has a superior renin inhibitory activity and is useful as an agent for the prophylaxis or treatment of hypertension, various organ damages attributable to hypertension, and the like.

### BACKGROUND OF INVENTION

Hypertension is one of representative lifestyle-related diseases. Hypertension which is left untreated for long time lays a heavy burden on the cardiovascular system and results in arteriosclerosis to progress, thus causing various disorders in important organs, such as cerebral hemorrhage, cerebral infarction, cardiac failure, angina pectoris, myocardial infarction, renal failure and the like. Accordingly, the purpose of treating hypertension lies not only in lowering the blood pressure, but also in improving and/or preventing disorders in important organs including brain, heart and kidney, by controlling the blood pressure. As a method of treating hypertension, there are available fundamental treatments based on improvement in the lifestyle, such as dietetic therapy, exercise therapy and the like, as well as an attempt to control the blood pressure by positive pharmaceutical intervention.

The renin-angiotensin (RA) system is a system of biosynthesis of angiotensin II (AII), which is a major vasopressor factor, and takes an important role in the control of the blood pressure and the amount of body fluid. AII exhibits a strong vasoconstrictive effect brought by the intervention of AII receptors on the cellular membrane, thus raising the blood pressure, and also promotes cellular propagation or production of extracellular matrix by directly acting on the AII receptors in the cardiac cells or renal cells. Therefore, drugs inhibiting increase in the activity of the RA system can be expected to have a blood pressure lowering action as well as a powerful organ protecting action, and thus active researches on such drugs have been conducted so far.

The method of inhibiting the AII action is broadly classified into methods of inhibiting the biosynthesis of AII and methods of inhibiting the binding of AII to AII receptors. For the drugs inhibiting the biosynthesis of AII, angiotensin converting enzyme (ACE) inhibitory drugs have been already put to practical use and are being confirmed to have a blood pressure lowering action as well as an effect for protecting various organs. However, since ACE is an enzyme identical to kininase II, which is a bradykinin degrading enzyme, ACE inhibitory drug inhibits the biosynthesis of AII as well as the degradation of bradykinin. As a result, ACE inhibitory drugs are believed to induce side effects such as dry cough, angioedema and the like, which are considered to be caused by accumulation of bradykinin.

As the drugs inhibiting the binding of AII to AII receptors, AII type 1 receptor blockers (ARB) have been developed. ARB has a merit in that it can inhibit, at the receptor level, the action of AII that is biosynthesized by not only ACE but also an enzyme other than ACE, such as chymase and the like. It is known that administration of ACE inhibitors and ARB increases the plasma renin activity (PRA) as a compensatory feedback effect, since these drugs act on a more peripheral region of the RA system.

Renin is an enzyme occupying a position at the uppermost stream of the RA system, and converts angiotensinogen to angiotensin I. A renin inhibitory drug inhibits the RA system by inhibiting the biosynthesis of AII in the same manner as the ACE inhibitory drugs do, and thus can be expected to have a blood pressure lowering action or an effect of protecting various organs. Since the renin inhibitory drug does not have influence on the metabolism of bradykinin, it is believed to have no risk of side effects such as dry cough and the like, that are observed with the ACE inhibitory drugs. Furthermore, while the ACE inhibitory drugs or ARB increase the PRA level, the renin inhibitory drugs are the only drugs that can reduce PRA.

As renin inhibitors, orally administrable Aliskiren has been reported (Chem. Biol., 2000, vol. 7, pages 493-504; Hypertension, 2003, vol. 42, pages 1137-1143; J. Hypertens., 2005, vol. 23, pages 417-426 etc.). In addition, low molecular weight renin inhibitory drugs are disclosed in WO 2004/002957, WO 2004/089915 and the like.

Imidazole compounds have been reported as orexin receptor antagonists (e.g., WO 2003/002559, WO 2003/002561, WO 2003/032991, WO 2003/041711, WO 2003/051368, WO 2003/051871, WO 2003/051873, WO 2004/026866, WO 2004/041791 etc.).

### DISCLOSURE OF THE INVENTION

There is a demand on the development of a novel compound having a superior renin inhibitory activity, which is useful as a pharmaceutical agent (e.g., hypertension, agent for the prophylaxis or treatment of various organ damages attributable to hypertension and the like, and the like).

The present inventors have conducted various studies, and as a result, first succeeded in the creation of a novel compound represented by the following formula (I) and a salt thereof, and found that the compound and a salt thereof unexpectedly have a superior renin inhibitory activity, and are useful as pharmaceutical agents, which resulted in the completion of the present invention.

Accordingly, the present invention relates to the following:

[1] a compound represented by the formula:

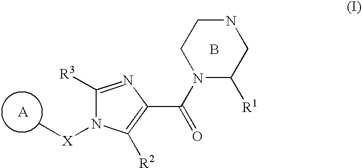

wherein
R¹ is a substituent,
R² is a cyclic group optionally having substituent(s), C₁₋₁₀ alkyl optionally having substituent(s), C₂₋₁₀ alkenyl optionally having substituent(s) or C₂₋₁₀ alkynyl optionally having substituent(s),
R³ is a hydrogen atom, a halogen atom, C₁₋₆ alkyl or C₁₋₆ alkoxy,
X is bond or spacer having 1 to 6 atoms in the main chain,
ring A is C₅₋₇ cycloalkane optionally having substituent(s), and
ring B is piperazine optionally further having substituent(s) besides R¹,
or a salt thereof [hereinafter to be sometimes abbreviated as compound (I)];
[2] the compound of the aforementioned [1], wherein R¹ is a hydrocarbon group optionally having substituent(s);
[3] the compound of the aforementioned [1], wherein R² is C₆₋₁₄ aryl optionally having substituent(s) or C₃₋₁₀ cycloalkyl optionally having substituent(s);
[4] the compound of the aforementioned [1], wherein R³ is a hydrogen atom, a halogen atom, C₁₋₃ alkyl or C₁₋₃ alkoxy;
[5] the compound of the aforementioned [1], wherein X is bond or C₁₋₆ alkylene optionally having substituent(s);
[6] the compound of the aforementioned [1], wherein ring A is C₅₋₇ cycloalkane optionally having substituent(s) selected from a halogen atom, a hydrocarbon group optionally having substituent(s), hydroxy optionally having a substituent and amino optionally having substituent(s);
[7] the compound of the aforementioned [1], wherein ring B is a ring represented by the formula:

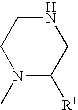

wherein R¹ is as defined above;
[8] a compound represented by the formula:

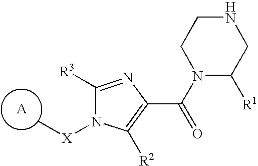

wherein

R¹ is

(a) C₁₋₆ alkyl substituted by hydroxy optionally having a substituent,
(b) C₁₋₆ alkyl substituted by phenylamino optionally having substituent(s), or
(c) C₇₋₁₃ aralkyl optionally having substituent(s);

R² is optionally halogenated C₆₋₁₀ aryl;

R³ is a hydrogen atom, a halogen atom, C₁₋₃ alkyl or C₁₋₃ alkoxy;

X is bond or C₁₋₆ alkylene optionally having substituent(s); and

ring A is

(a) C₅₋₇ cycloalkane substituted by hydroxy optionally having a substituent, and optionally further substituted by C₁₋₃ alkyl optionally having substituent(s), or
(b) C₅₋₇ cycloalkane substituted by amino optionally having substituent(s);
[9] (1S,2R)-1-(Methoxymethyl)-2-{4-[((2R)-2-{2-[(2-methyl-1,3-benzothiazol-5-yl)oxy]ethyl}piperazin-1-yl)carbonyl]-5-phenyl-1H-imidazol-1-yl}cyclohexanol or a salt thereof;
[10] Methyl [(1S,2S)-2-(4-{[(2R)-2-(3,5-difluorobenzyl)piperazin-1-yl]carbonyl}-5-phenyl-1H-imidazol-1-yl)cyclohexyl]carbamate or a salt thereof;
[11] (1S,2R)-1-(Methoxymethyl)-2-[5-phenyl-4-({(2R)-2-[(5-phenyl-1,3,4-oxadiazol-2-yl)methyl]piperazin-1-yl}carbonyl)-1H-imidazol-1-yl]cyclohexanol or a salt thereof;
[12] (1S,2R)-1-(Methoxymethyl)-2-[4-({(2R)-2-[2-(2-methoxy-4-methylphenoxy)ethyl]piperazin-1-yl}carbonyl)-5-phenyl-1H-imidazol-1-yl]cyclohexanol or a salt thereof;
[13] Ethyl [(1S,2S)-2-(4-{[(2R)-2-(3,5-difluorobenzyl)piperazin-1-yl]carbonyl}-5-phenyl-1H-imidazol-1-yl)cyclohexyl]carbamate or a salt thereof;
[14] (1S,2R)-2-(4-{[(2R)-2-Benzylpiperazin-1-yl]carbonyl}-5-phenyl-1H-imidazol-1-yl)-1-(methoxymethyl)cyclohexanol or a salt thereof;
[15] (1S,2R)-2-[4-{[(2R)-2-Benzylpiperazin-1-yl]carbonyl}-5-(3-fluorophenyl)-1H-imidazol-1-yl]-1-(methoxymethyl)cyclohexanol or a salt thereof;
[16] Methyl [(1S,2S)-2-(4-{[(2R)-2-(2-anilinoethyl)piperazin-1-yl]carbonyl}-5-phenyl-1H-imidazol-1-yl)cyclohexyl]carbamate or a salt thereof;
[17] (1S,2R)-1-(Methoxymethyl)-2-(4-{[(2R)-2-(2-morpholinobenzyl)piperazin-1-yl]carbonyl}-5-phenyl-1H-imidazol-1-yl)cyclohexanol or a salt thereof;
[18] (1S,2R)-2-(4-{[(2R)-2-Benzylpiperazin-1-yl]carbonyl}-5-phenyl-1H-imidazol-1-yl)-1-methylcyclohexanol or a salt thereof;
[19] (1S,2R)-1-(Methoxymethyl)-2-{4-[((2R)-2-{2-[(3-methoxyphenyl)amino]ethyl}piperazin-1-yl)carbonyl]-5-phenyl-1H-imidazol-1-yl}cyclohexanol or a salt thereof;
[20] (1S,2R)-1-(Methoxymethyl)-2-(5-phenyl-4-{[(2R)-2-(2-{[4-(1H-pyrazol-1-yl)phenyl]amino}ethyl)piperazin-1-yl]carbonyl}-1H-imidazol-1-yl)cyclohexanol or a salt thereof;
[21] (1S,2R)-1-(Methoxymethyl)-2-{4-[((2R)-2-{2-[(5-methoxy-2-methylphenyl)amino]ethyl}piperazin-1-yl)carbonyl]-5-phenyl-1H-imidazol-1-yl}cyclohexanol or a salt thereof;
[22] (1S,2R)-2-{4-[((2R)-2-{2-[(2-Ethyl-1,3-benzoxazol-5-yl)amino]ethyl}piperazin-1-yl)carbonyl]-5-phenyl-1H-imidazol-1-yl}-1-(methoxymethyl)cyclohexanol or a salt thereof;
[23] 1-[(4-{[(2R)-2-(2-Anilinoethyl)piperazin-1-yl]carbonyl}-5-phenyl-1H-imidazol-1-yl)methyl]cyclohexanol or a salt thereof;
[24] a prodrug of the compound of the aforementioned [1];
[25] a pharmaceutical agent comprising the compound of the aforementioned [1] or a prodrug thereof;
[26] the pharmaceutical agent of the aforementioned [25], which is a renin inhibitor;
[27] the pharmaceutical agent of the aforementioned [25], which is an agent for the prophylaxis or treatment of hypertension;
[28] the pharmaceutical agent of the aforementioned [25], which is an agent for the prophylaxis or treatment of various organ damages attributable to hypertension;
[29] a method for the prophylaxis or treatment of hypertension in a mammal, which comprises administering an effective amount of the compound of the aforementioned [1] or a prodrug thereof to the mammal;
[30] use of the compound of the aforementioned [1] or a prodrug thereof for the production of an agent for the prophylaxis or treatment of hypertension; and the like.

### EFFECT OF THE INVENTION

Compound (I) has a superior renin inhibitory activity, and thus it is useful as an agent for the prophylaxis or treatment of hypertension, various organ damages attributable to hypertension, and the like.

### DETAILED DESCRIPTION OF THE INVENTION

Examples of the “halogen atom” in the present specification include fluorine, chlorine, bromine and iodine.

Examples of the “C₁₋₄ alkylenedioxy” in the present specification include methylenedioxy, ethylenedioxy, trimethylenedioxy and the like.

Examples of the “C₁₋₆ alkyl” in the present specification include methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl and the like.

Examples of the “C₁₋₆ alkoxy” in the present specification include methoxy, ethoxy, propoxy, isopropoxy, butoxy, isobutoxy, sec-butoxy, tert-butoxy and the like.

Examples of the “C₁₋₆ alkoxy-carbonyl” in the present specification include methoxycarbonyl, ethoxycarbonyl, propoxycarbonyl, tert-butoxycarbonyl and the like.

Examples of the “C₁₋₆ alkyl-carbonyl” in the present specification include acetyl, propanoyl, butanoyl, isobutanoyl, pentanoyl, isopentanoyl, hexanoyl and the like.

The “optionally halogenated” in the present specification means being optionally substituted by 1 to 5, preferably 1 to 3, halogen atoms.

Examples of the “hydrocarbon group” of the “hydrocarbon group optionally having substituent(s)” in the present specification include C₁₋₁₀ alkyl, C₂₋₁₀ alkenyl, C₂₋₁₀ alkynyl, C₁₋₃ alkylidene, C₃₋₁₀ cycloalkyl, C₃₋₁₀ cycloalkenyl, C₄₋₁₀ cycloalkadienyl, C₆₋₁₄ aryl, C₇₋₁₃ aralkyl, C₈₋₁₃ arylalkenyl, C₃₋₁₀ cycloalkyl-C₁₋₆ alkyl and the like. The above-mentioned C₃₋₁₀ cycloalkyl, C₃₋₁₀ cycloalkenyl and C₄₋₁₀ cycloalkadienyl are each optionally condensed with a benzene ring.

Examples of the “C₁₋₁₀ alkyl” in the present specification include methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, heptyl, octyl, nonyl, decyl and the like. Among these, C₁₋₆ alkyl is preferable.

Examples of the “C₂₋₁₀ alkenyl” in the present specification include ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl, 5-hexenyl, 1-heptenyl, 1-octenyl and the like. Among these, C₂₋₆ alkenyl is preferable.

Examples of the “C₂₋₁₀ alkynyl” in the present specification include ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 1-heptynyl, 1-octynyl and the like. Among these, C₂₋₆ alkynyl is preferable.

Examples of the “C₁₋₃ alkylidene” in the present specification include methylene, ethylidene, propylidene, isopropylidene and the like.

Examples of the “C₃₋₁₀ cycloalkyl” in the present specification include cyclopropyl, cyclobutyl, cyclopentyl, cyclohexyl, cycloheptyl, cyclooctyl, bicyclo[2.2.1]heptyl, bicyclo[2.2.2]octyl, bicyclo[3.2.1]octyl, bicyclo[3.2.2]nonyl, bicyclo[3.3.1]nonyl, bicyclo[4.2.1]nonyl, bicyclo[4.3.1]decyl, adamantyl and the like. Among these, C₃₋₆ cycloalkyl is preferable. The above-mentioned C₃₋₁₀ cycloalkyl is optionally condensed with a benzene ring. Examples of the condensed group include indanyl, tetrahydronaphthyl, fluorenyl and the like.

Examples of the “C₃₋₁₀ cycloalkenyl” in the present specification include 2-cyclopenten-1-yl, 3-cyclopenten-1-yl, 2-cyclohexen-1-yl, 3-cyclohexen-1-yl and the like. The above-mentioned C₃₋₁₀ cycloalkenyl is optionally condensed with a benzene ring. Examples of the condensed group include indenyl and the like.

Examples of the “C₄₋₁₀ cycloalkadienyl” in the present specification include 2,4-cyclopentadien-1-yl, 2,4-cyclohexadien-1-yl, 2,5-cyclohexadien-1-yl and the like. The above-mentioned C₄₋₁₀ cycloalkadienyl is optionally condensed with a benzene ring.

Examples of the “C₆₋₁₄ aryl” in the present specification include phenyl, 1-naphthyl, 2-naphthyl, biphenylyl, 2-anthryl and the like. Among these, C₆₋₁₀ aryl is preferable, and phenyl is more preferable. The above-mentioned C₆₋₁₄ aryl is optionally condensed with C₃₋₁₀ cycloalkane (examples of the C₃₋₁₀ cycloalkane include rings corresponding to the above-mentioned C₃₋₁₀ cycloalkyl). Examples of the condensed group include tetrahydronaphthyl and the like.

Examples of the “C₇₋₁₃ aralkyl” in the present specification include benzyl, phenethyl, naphthylmethyl, biphenylylmethyl and the like.

Examples of the “C₈₋₁₃ arylalkenyl” in the present specification include styryl and the like.

Examples of the “C₃₋₁₀ cycloalkyl-C₁₋₆ alkyl” in the present specification include cyclopropylmethyl, cyclohexylmethyl and the like.

The “hydrocarbon group” of the “hydrocarbon group optionally having substituent(s)” optionally have substituent(s) (e.g., 1 to 5, preferably 1 to 3 substituents) at substitutable position(s). When the number of the substituents is not less than 2, respective substituents may be the same or different.

Examples of the “substituent” of the “hydrocarbon group optionally having substituent(s)” include the following substituents.

(1) a halogen atom;
(2) C₃₋₁₀ cycloalkyl (e.g., cyclopropyl, cyclohexyl);
(3) C₆₋₁₄ aryl (e.g., phenyl, naphthyl) optionally having 1 to 3 substituents selected from
(i) carboxy,
(ii) hydroxy,
(iii) C₁₋₆ alkyl optionally having 1 to 3 substituents selected from
   (a) hydroxy, and
   (b) a halogen atom,
(iv) C₁₋₆ alkoxy optionally having 1 to 3 substituents selected from
   (a) C₁₋₆ alkoxy,
   (b) carbamoyl optionally mono- or di-substituted by substituent(s) selected from C₁₋₆ alkyl optionally substituted by carbamoyl, and C₁₋₆ alkylsulfonyl,
   (c) carboxyl,
   (d) C₁₋₆ alkoxy-carbonyl optionally substituted by a non-aromatic heterocyclic group (e.g., dioxolyl) optionally having 1 to 3 substituents selected from oxo and C₁₋₆ alkyl,
   (e) cyano, and
   (f) a non-aromatic heterocyclic group (e.g., oxadiazolinyl) optionally substituted by oxo,
(v) carbamoyl optionally mono- or di-substituted by substituent(s) selected from
   (a) C₁₋₆ alkyl optionally substituted by hydroxy, and
   (b) C₁₋₆ alkylsulfonyl,
(vi) a non-aromatic heterocyclic group (e.g., oxadiazolinyl) optionally substituted by oxo,
(vii) an aromatic heterocyclic group (e.g., tetrazolyl),
(viii) C₁₋₆ alkoxy-carbonyl optionally substituted by a non-aromatic heterocyclic group (e.g., dioxolyl) optionally having 1 to 3 substituents selected from oxo and C₁₋₆ alkyl,
(ix) cyano,
(x) sulfamoyl,
(xi) halogen,
(xii) C₁₋₆ alkylsulfonyl (e.g., methylsulfonyl), and
(xiii) C₁₋₆ alkyl sulfonyloxy (e.g., methylsulfonyloxy);
   (4) an aromatic heterocyclic group (e.g., thienyl, furyl, pyrrolyl, pyrazolyl, triazolyl, pyridyl, oxazolyl, thiazolyl, tetrazolyl, oxadiazolyl, pyrazinyl, quinolyl, indolyl, imidazolyl, indazolyl, benzimidazolyl, benzotriazolyl) optionally having 1 to 3 substituents selected from
(i) a halogen atom,
(ii) C₁₋₆ alkyl optionally having 1 to 3 substituents selected from
   (a) a halogen atom,
   (b) hydroxy,
   (c) C₆₋₁₄ aryl (e.g., phenyl),
   (d) C₁₋₆ alkoxy,
   (e) C₁₋₆ alkyl-carbonyloxy, and
   (f) a non-aromatic heterocyclic group (the non-aromatic heterocyclic group is optionally oxidized; e.g., tetrahydrofuryl) optionally having 1 to 3 C₁₋₆ alkyl,
(iii) C₃₋₆ cycloalkyl,
(iv) C₆₋₁₄ aryl,
(v) hydroxy,
(vi) C₁₋₆ alkoxy,
(vii) C₁₋₆ alkyl-carbonyl optionally having amino optionally mono- or di-substituted by C₁₋₆ alkyl-carbonyl,
(viii) C₆₋₁₄ aryl-carbonyl (e.g., benzoyl),
(ix) C₁₋₆ alkoxy-carbonyl,
(x) carboxy,
(xi) carbamoyl optionally mono- or di-substituted by C₁₋₆ alkyl optionally having 1 to 3 substituents selected from hydroxy and carbamoyl,
(xii) C₁₋₆ alkylsulfonyl,
(xiii) C₆₋₁₄ arylsulfonyl, and
(xiv) cyano;
   (5) a non-aromatic heterocyclic group (the non-aromatic heterocyclic group is optionally oxidized; e.g., tetrahydrofuryl, morpholinyl, thiomorpholinyl, piperidinyl, pyrrolidinyl, piperazinyl, dioxolyl, dioxolanyl, 1,3-dihydro-2-benzofuranyl, thiazolidinyl, oxadiazolidinyl, 1-oxidothiomorpholinyl, 1,1-dioxidothiomorpholinyl, tetrahydropyranyl, dihydroisoindolyl, dihydroindazolyl, tetrahydroindazolyl, dihydrobenzimidazolyl, dihydrobenzoxazolyl, dihydrobenzoxazinyl, tetrahydroquinolyl, tetrahydroisoquinolyl) optionally having 1 to 3 substituents selected from
(i) a halogen atom,
(ii) C₁₋₆ alkyl optionally having 1 to 3 substituents selected from
   (a) a halogen atom,
   (b) hydroxy,
   (c) C₆₋₁₄ aryl (e.g., phenyl),
   (d) C₁₋₆ alkoxy,
   (e) C₁₋₆ alkyl-carbonyloxy, and
   (f) a non-aromatic heterocyclic group (the non-aromatic heterocyclic group is optionally oxidized; e.g., tetrahydrofuryl) optionally having 1 to 3 C₁₋₆ alkyl,
(iii) C₃₋₆ cycloalkyl,
(iv) C₆₋₁₄ aryl,
(v) hydroxy,
(vi) C₁₋₆ alkoxy,
(vii) C₁₋₆ alkyl-carbonyl optionally having amino optionally mono- or di-substituted by C₁₋₆ alkyl-carbonyl,
(viii) C₆-C₁₄ aryl-carbonyl (e.g., benzoyl),
(ix) C₁₋₆ alkoxy-carbonyl,
(x) carboxy,
(xi) carbamoyl optionally mono- or di-substituted by C₁₋₆ alkyl optionally having 1 to 3 substituents selected from hydroxy and carbamoyl,
(xii) C₁₋₆ alkylsulfonyl,
(xiii) C₆₋₁₄ arylsulfonyl,
(xiv) cyano, and
(xv) oxo;
   (6) amino optionally mono- or di-substituted by substituent(s) selected from
(i) C₁₋₁₀ alkyl optionally substituted by 1 to 3 substituents selected from
   (a) hydroxy,
   (b) C₁₋₆ alkoxy optionally substituted by C₆₋₁₄ aryl (e.g., phenyl),
   (c) carboxy,
   (d) C₃₋₁₀ cycloalkyl (e.g., cyclopropyl) optionally substituted by C₁₋₆ alkoxy-carbonyl,
   (e) a halogen atom,
   (f) an aromatic heterocyclic group (e.g., furyl, pyridyl, indolyl, imidazolyl, thienyl, pyrazolyl, pyrrolyl) optionally having 1 to 3 substituents selected from
      1) C₁₋₆ alkyl optionally substituted by hydroxy,
      2) C₁₋₆ alkoxy-carbonyl,
      3) carboxy,
      4) a halogen atom, and
      5) C₁₋₆ alkylthio,
   (g) C₆₋₁₄ aryl (e.g., phenyl) optionally having 1 to 3 substituents selected from
      1) amino optionally mono- or di-substituted by substituent(s) selected from C₁₋₆ alkyl and C₁₋₆ alkyl-carbonyl,
      2) C₁₋₄ alkylenedioxy,
      3) hydroxy, and
      4) C₁₋₆ alkoxy optionally substituted by carboxy,
   (h) C₁₋₆ alkylthio,
   (i) C₁₋₆ alkylsulfonyl,
   (j) amino optionally mono- or di-substituted by C₁₋₆ alkoxy-carbonyl optionally substituted by C₆₋₁₄ aryl (e.g., phenyl), and
   (k) carbamoyl,
(ii) C₆₋₁₄ aryl (e.g., phenyl) optionally having 1 to 3 substituents selected from
   (a) a halogen atom,
   (b) optionally halogenated C₁₋₆ alkyl (e.g., isopropyl, trifluoromethyl),
   (c) optionally halogenated C₁₋₆ alkoxy (e.g., methoxy, trifluoromethoxy, difluoromethoxy),
   (d) cyano,
   (e) nitro,
   (f) carboxy,
   (g) C₁₋₆ alkyl-carbonyl,
   (h) C₁₋₆ alkoxy-carbonyl,
   (i) C₁₋₄ alkylenedioxy, and
   (j) a 5- or 6-membered heterocyclic group (e.g., pyrazolyl, piperidinyl, dihydropyridyl) optionally having 1 or 2 oxo,
(iii) C₃₋₆ cycloalkyl optionally condensed with a benzene ring (e.g., cyclopropyl, cyclohexyl, indanyl),
(iv) C₇₋₁₃ aralkyl (e.g., benzyl),
(v) C₁₋₆ alkyl-carbonyl optionally having 1 to 3 substituents selected from
   (a) carboxy,
   (b) C₆₋₁₄ aryl (e.g., phenyl),
   (c) amino optionally mono- or di-substituted by C₁₋₆ alkyl-carbonyl,
   (d) C₁₋₆ alkoxy optionally substituted by C₁₋₆ alkoxy,
   (e) an aromatic heterocyclic group (e.g., thienyl),
   (f) C₁₋₆ alkoxy-carbonyl,
   (g) carbamoyl optionally mono- or di-substituted by C₃₋₁₀ cycloalkyl, and
   (h) non-aromatic heterocyclylcarbonyl (e.g., morpholinylcarbonyl),
(vi) C₃₋₁₀ cycloalkyl-carbonyl,
(vii) C₁₋₆ alkoxy-carbonyl optionally having 1 or 2 C₆₋₁₄ aryl (e.g., phenyl),
(viii) C₆₋₁₄ aryl-carbonyl (e.g., benzoyl) optionally having 1 to 3 substituents selected from a halogen atom and C₁₋₆ alkoxy,
(ix) C₇₋₁₃ aralkyl-carbonyl (e.g., benzylcarbonyl, phenethylcarbonyl),
(x) carbamoyl optionally mono- or di-substituted by C₁₋₆ alkyl optionally having 1 to 3 substituents selected from
   (a) carboxy,
   (b) C₁₋₆ alkoxy-carbonyl, and
   (c) carbamoyl,
(xi) C₆₋₁₄ aryl-carbamoyl (e.g., phenylcarbamoyl, 1-naphthylcarbamoyl, 2-naphthylcarbamoyl),
(xii) C₇₋₁₃ aralkyl-carbamoyl (e.g., benzylcarbamoyl),
(xiii) C₁₋₆ alkylsulfonyl (e.g., methylsulfonyl, ethylsulfonyl, isopropylsulfonyl),
(xiv) C₆₋₁₄ arylsulfonyl (e.g., benzenesulfonyl, toluenesulfonyl, 1-naphthalenesulfonyl, 2-naphthalenesulfonyl),
(xv) C₇₋₁₃ aralkylsulfonyl (e.g., benzylsulfonyl), and
(xvi) a heterocyclic group (the heterocyclic group is optionally oxidized; e.g., tetrahydrofuryl, tetrahydropyranyl, pyridyl, benzoxazolyl, benzothiazolyl, benzimidazolyl, benzothienyl, benzofuranyl, benzotriazolyl, benzisoxazolyl, benzisothiazolyl, dihydrobenzofuranyl, dihydrobenzoxazolyl, indolyl, indazolyl, dihydrofuropyridyl, tetrahydroquinolyl, tetrahydroisoquinolyl, chromenyl, thienopyridyl, imidazopyridyl, pyrazolopyridyl, pyrrolopyrazinyl, imidazopyrazinyl, pyrazolothienyl, dihydrobenzoxazinyl) optionally substituted by 1 to 3 substituents selected from hydroxy, C₁₋₆ alkyl and oxo;
   (7) amidino;
   (8) C₁₋₆ alkyl-carbonyl optionally having 1 to 3 substituents selected from a halogen atom and hydroxy;
   (9) C₁₋₆ alkoxy-carbonyl optionally having 1 to 3 substituents selected from a halogen atom and C₆₋₁₄ aryl (e.g., phenyl);
   (10) carbamoyl optionally mono- or di-substituted by substituent(s) selected from
(i) C₁₋₆ alkyl optionally having 1 to 3 substituents selected from a halogen atom, hydroxy, carbamoyl and an aromatic heterocyclic group (e.g., furyl),
(ii) C₆₋₁₄ aryl (e.g., phenyl),
(iii) C₇₋₁₃ aralkyl (e.g., benzyl), and
(iv) aromatic heterocyclyl-C₁₋₆ alkyl (e.g., furfuryl);
   (11) thiocarbamoyl optionally mono- or di-substituted by C₁₋₆ alkyl optionally substituted by 1 to 3 halogen atoms;
   (12) sulfamoyl optionally mono- or di-substituted by C₁₋₆ alkyl optionally substituted by 1 to 3 halogen atoms;
   (13) carboxy;
   (14) hydroxy;
   (15) C₁₋₆ alkoxy optionally having 1 to 3 substituents selected from
(i) a halogen atom,
(ii) carboxy,
(iii) hydroxy,
(iv) C₁₋₆ alkoxy optionally having 1 or 2 hydroxy,
(v) C₆₋₁₄ aryl (e.g., phenyl) optionally substituted by C₁₋₆ alkylsulfonyl,
(vi) C₃₋₆ cycloalkyl,
(vii) C₁₋₆ alkoxy-carbonyl,
(viii) C₁₋₆ alkylsulfonyl (e.g., methylsulfonyl),
(ix) mono- or di-C₁₋₆ alkylamino,
(x) C₁₋₆ alkyl-carbonylamino,
(xi) C₁₋₆ alkylthio,
(xii) a heterocyclic group (e.g., a 5- or 6-membered heterocyclic group such as thiazolyl, imidazolyl, pyrrolidinyl, imidazolidinyl, oxazolidinyl, pyridyl, oxetanyl, tetrahydrothiopyranyl, tetrahydropyranyl and the like; benzimidazolyl; the heterocyclic group is optionally oxidized, e.g., 1,1-dioxidotetrahydrothiopyranyl, 1,1-dioxidothiomorpholinyl) optionally having 1 to 3 substituents selected from C₁₋₆ alkyl and oxo, and
(xiii) carbamoyl optionally mono- or di-substituted by C₁₋₆ alkyl optionally having 1 to 3 substituents selected from carbamoyl and hydroxy;
   (16) C₂₋₆ alkenyloxy (e.g., ethenyloxy) optionally substituted by 1 to 3 halogen atoms;
   (17) C₃₋₁₀ cycloalkyloxy optionally condensed with a benzene ring (e.g., cyclohexyloxy, tetrahydronaphthyloxy, indanyloxy) optionally having oxo;
   (18) C₇₋₁₃ aralkyloxy (e.g., benzyloxy);
   (19) C₆₋₁₄ aryloxy (e.g., phenyloxy, naphthyloxy; the C₆₋₁₄ aryl is optionally condensed with C₃₋₁₀ cycloalkane) optionally having 1 to 3 substituents selected from
(i) a halogen atom,
(ii) cyano,
(iii) C₁₋₆ alkyl optionally having 1 or 2 substituents selected from a halogen atom, carboxy, hydroxy, C₁₋₆ alkoxy-carbonyl and mono- or di-C₁₋₆ alkylamino,
(iv) C₁₋₆ alkoxy optionally having 1 to 3 substituents selected from a halogen atom and C₁₋₆ alkoxy,
(v) C₁₋₄ alkylenedioxy,
(vi) carboxy,
(vii) C₁₋₆ alkyl-carbonyl,
(viii) C₁₋₆ alkoxy-carbonyl,
(ix) 5- or 6-membered non-aromatic heterocyclylcarbonyl (e.g., azetidinylcarbonyl),
(x) carbamoyl,
(xi) optionally halogenated mono- or di-C₁₋₆ alkyl-carbamoyl,
(xii) C₃₋₆ cycloalkyl-carbamoyl (e.g., cyclopropylcarbamoyl),
(xiii) mono- or di-C₁₋₆ alkylamino,
(xiv) optionally halogenated C₁₋₆ alkylsulfonyl (e.g., methylsulfonyl, trifluoromethylsulfonyl),
(xv) a 5- or 6-membered heterocyclic group (e.g., imidazolyl, pyrazolyl, triazolyl, oxadiazolyl, pyrrolidinyl, piperazinyl, morpholinyl) optionally having 1 or 2 substituents selected from C₁₋₆ alkyl, C₁₋₆ alkyl-carbonyl, C₁₋₆ alkoxy-carbonyl, C₁₋₄ alkylenedioxy and oxo, and
(xvi) a 9- or 10-membered fused heterocyclic group (e.g., dihydroimidazoimidazolyl) optionally having 1 to 3 substituents selected from C₁₋₆ alkyl and oxo;
   (20) C₃₋₁₀ cycloalkyl-C₁₋₆ alkyloxy (e.g., cyclopropylmethyloxy);
   (21) heterocyclyloxy (e.g., 5- or 6-membered aromatic heterocyclyloxy such as pyrazolyloxy, triazolyloxy, thienyloxy, thiazolyloxy, isoxazolyloxy, oxadiazolyloxy, pyridyloxy, pyrimidinyloxy and the like; 5- or 6-membered non-aromatic heterocyclyloxy such as piperidinyloxy, tetrahydropyranyloxy, tetrahydrothiopyranyloxy, 1-oxidotetrahydrothiopyranyloxy, 1,1-dioxidotetrahydrothiopyranyloxy and the like; 9- or 10-membered fused heterocyclyloxy such as benzothienyloxy, benzothiazolyloxy, benzofuranyloxy, benzimidazolyloxy, benzoxazolyloxy, dihydrobenzoxazolyloxy, benzisoxazolyloxy, benzisothiazolyloxy, dihydrobenzofuranyloxy, dihydroquinolyloxy, dihydroisoquinolyloxy, tetrahydroquinolyloxy, tetrahydroisoquinolyloxy, chromenyloxy, thienopyridyloxy, benzotriazolyloxy, indolyloxy, indazolyloxy, imidazopyridyloxy, pyrazolopyridyloxy, pyrrolopyrazinyloxy, imidazopyrazinyloxy, pyrazolothienyloxy, dihydrofuropyridyloxy, dihydrobenzoxazinyloxy and the like; the heterocycle is optionally oxidized) optionally having 1 to 3 substituents selected from
(i) a halogen atom,
(ii) cyano,
(iii) C₁₋₆ alkyl optionally having 1 to 3 substituents selected from a halogen atom, C₁₋₆ alkoxy and C₁₋₆ alkoxy-carbonyl,
(iv) C₁₋₆ alkoxy-carbonyl-C₁₋₆ alkyl,
(v) mono- or di-C₁₋₆ alkylamino-C₁₋₆ alkyl (e.g., dimethylaminomethyl),
(vi) C₆₋₁₀ aryl (e.g., phenyl),
(vii) C₃₋₁₀ cycloalkyl,
(viii) C₁₋₆ alkoxy,
(ix) C₁₋₆ alkoxy-carbonyl,
(x) carboxy, and
(xi) oxo;
   (22) C₁₋₆ alkyl-carbonyloxy (e.g., acetyloxy, tert-butylcarbonyloxy);
   (23) mercapto;
   (24) C₁₋₆ alkylthio (e.g., methylthio, ethylthio) optionally substituted by 1 to 3 halogen atoms;
   (25) C₇₋₂₀ aralkylthio (e.g., benzylthio, tritylthio);
   (26) C₆₋₁₄ arylthio (e.g., phenylthio, naphthylthio);
   (27) sulfo;
   (28) C₁₋₆ alkylsulfinyl (e.g., methylsulfinyl);
   (29) C₆₋₁₄ arylsulfinyl (e.g., phenylsulfinyl);
   (30) C₁₋₆ alkylsulfonyl (e.g., methylsulfonyl) optionally substituted by 1 to 3 halogen atoms;
   (31) C₆₋₁₄ arylsulfonyl (e.g., phenylsulfonyl) optionally substituted by C₁₋₆ alkoxy;
   (32) C₃₋₁₀ cycloalkylsulfonyl (e.g., cyclopropylsulfonyl);
   (33) aromatic heterocyclylsulfonyl (e.g., pyridylsulfonyl, pyrazolylsulfonyl, thienylsulfonyl, furylsulfonyl, imidazolylsulfonyl) optionally having 1 to 3 substituents selected from
(i) C₁₋₆ alkyl,
(ii) C₁₋₆ alkoxy,
(iii) C₁₋₆ alkoxy-carbonyl, and
(iv) a halogen atom;
   (34) cyano;
   (35) azido;
   (36) nitro;
   (37) nitroso;
   (38) oxo;
   (39) non-aromatic heterocyclylcarbonyl (the non-aromatic heterocycle is optionally oxidized; e.g., morpholinylcarbonyl, piperazinylcarbonyl) optionally substituted by C₁₋₆ alkyl optionally substituted by C₆₋₁₄ aryl (e.g., phenyl);
   (40) non-aromatic heterocyclylcarbonyloxy (e.g., pyrrolidinylcarbonyloxy);
   (41) C₁₋₄ alkylenedioxy optionally substituted by 1 to 3 halogen atoms;
   (42) hydroxyimino optionally substituted by C₁₋₆ alkyl;
   (43) C₁₋₆ alkyl optionally having 1 to 5 (preferably 1 to 3) substituents selected from
(i) a halogen atom,
(ii) carboxy,
(iii) hydroxy,
(iv) C₁₋₆ alkoxy,
(v) C₁₋₆ alkoxy-carbonyl,
(vi) C₁₋₆ alkyl-carbonyloxy (e.g., acetyloxy, tert-butylcarbonyloxy),
(vii) amino,
(viii) carbamoyl optionally mono- or di-substituted by C₁₋₆ alkyl optionally substituted by hydroxy,
(ix) a non-aromatic heterocyclic group (the non-aromatic heterocyclic group is optionally oxidized; e.g., piperidino, tetrahydrofuryl) optionally substituted by C₁₋₆ alkyl,
(x) non-aromatic heterocyclylcarbonyl (the non-aromatic heterocycle is optionally oxidized; e.g., morpholinylcarbonyl),
(xi) C₆₋₁₄ aryl (e.g., phenyl) optionally substituted by C₁₋₆ alkylsulfonyl,
(xii) C₃₋₁₀ cycloalkyl (e.g., cyclopropyl), and
(xiii) an aromatic heterocyclic group (e.g., furyl) optionally having 1 to 3 substituents selected from carboxy and C₁₋₆ alkoxy-carbonyl;
   (44) C₂₋₆ alkenyl (e.g., ethenyl, 1-propenyl) optionally having 1 to 3 substituents selected from
(i) a halogen atom,
(ii) carboxy,
(iii) C₁₋₆ alkoxy-carbonyl,
(iv) carbamoyl, and
(v) C₆₋₁₄ aryl (e.g., phenyl) optionally substituted by C₁₋₆ alkoxy-carbonyl;
   (45) C₇₋₁₃ aralkyl (e.g., benzyl) optionally having 1 to 3 substituents selected from
(i) C₁₋₆ alkyl optionally substituted by 1 to 3 halogen atoms,
(ii) hydroxy,
(iii) C₁₋₆ alkoxy, and
(iv) a halogen atom;
   (46) C₆₋₁₀ aryl-carbamoyl (e.g., phenylcarbamoyl);
   (47) C₆₋₁₀ arylsulfinyl (e.g., phenylsulfinyl);
   (48) optionally halogenated C₆₋₁₀ arylsulfonyl (e.g., phenylsulfonyl, fluorophenylsulfonyl);
   (49) heterocyclylthio (e.g., thiazolylthio, thiadiazolylthio, triazolylthio, benzothiazolylthio, benzimidazolylthio, thiazolopyridylthio) optionally having C₁₋₆ alkyl optionally having 1 to 3 substituents selected from hydroxy and C₁₋₆ alkyl-carbonyloxy; and the like.

Examples of the “cyclic group” of the “cyclic group optionally having substituent(s)” in the present specification include an aromatic group, a non-aromatic cyclic group and the like.

Examples of the “aromatic group” include an aromatic hydrocarbon group and an aromatic heterocyclic group.

Examples of the “aromatic hydrocarbon group” include C₆₋₁₄ aryl and the like.

Examples of the “aromatic heterocyclic group” include a 4- to 7-membered (preferably 5- or 6-membered) monocyclic aromatic heterocyclic group containing, as a ring-constituting atom besides carbon atoms, 1 to 4 heteroatoms selected from an oxygen atom, a sulfur atom and a nitrogen atom, and a fused aromatic heterocyclic group. Examples of the fused aromatic heterocyclic group include a group derived from a fused ring wherein a ring corresponding to such 4- to 7-membered monocyclic aromatic heterocyclic group, and 1 or 2 rings selected from a 5- or 6-membered aromatic heterocycle containing 1 or 2 nitrogen atoms, a 5-membered aromatic heterocycle containing one sulfur atom and a benzene ring are condensed, and the like.

Examples of the “aromatic heterocyclic group” include 4- to 7-membered (preferably 5- or 6-membered) monocyclic aromatic heterocyclic groups such as furyl (e.g., 2-furyl, 3-furyl), thienyl (e.g., 2-thienyl, 3-thienyl), pyridyl (e.g., 2-pyridyl, 3-pyridyl, 4-pyridyl), pyrimidinyl (e.g., 2-pyrimidinyl, 4-pyrimidinyl, 5-pyrimidinyl), pyridazinyl (e.g., 3-pyridazinyl, 4-pyridazinyl), pyrazinyl (e.g., 2-pyrazinyl), pyrrolyl (e.g., 1-pyrrolyl, 2-pyrrolyl, 3-pyrrolyl), imidazolyl (e.g., 1-imidazolyl, 2-imidazolyl, 4-imidazolyl, 5-imidazolyl), pyrazolyl (e.g., 1-pyrazolyl, 3-pyrazolyl, 4-pyrazolyl), thiazolyl (e.g., 2-thiazolyl, 4-thiazolyl, 5-thiazolyl), isothiazolyl (e.g., 3-isothiazolyl, 4-isothiazolyl, 5-isothiazolyl), oxazolyl (e.g., 2-oxazolyl, 4-oxazolyl, 5-oxazolyl), isoxazolyl (e.g., 3-isoxazolyl, 4-isoxazolyl, 5-isoxazolyl), oxadiazolyl (e.g., 1,2,4-oxadiazol-5-yl, 1,3,4-oxadiazol-2-yl), thiadiazolyl (e.g., 1,2,4-thiadiazol-5-yl, 1,3,4-thiadiazol-2-yl), triazolyl (e.g., 1,2,4-triazol-1-yl, 1,2,4-triazol-3-yl, 1,2,3-triazol-1-yl, 1,2,3-triazol-2-yl, 1,2,3-triazol-4-yl), tetrazolyl (e.g., tetrazol-1-yl, tetrazol-5-yl), triazinyl (e.g., 1,3,5-triazin-2-yl, 1,3,5-triazin-4-yl, 1,2,3-triazin-4-yl, 1,2,4-triazin-3-yl) and the like;

fused aromatic heterocyclic groups such as quinolyl (e.g., 2-quinolyl, 3-quinolyl, 4-quinolyl, 6-quinolyl), isoquinolyl (e.g., 3-isoquinolyl), quinazolyl (e.g., 2-quinazolyl, 4-quinazolyl), quinoxalyl (e.g., 2-quinoxalyl, 6-quinoxalyl), benzofuranyl (e.g., 2-benzofuranyl, 3-benzofuranyl), benzothienyl (e.g., 2-benzothienyl, 3-benzothienyl), benzoxazolyl (e.g., 2-benzoxazolyl), benzisoxazolyl (e.g., 7-benzisoxazolyl), benzothiazolyl (e.g., 2-benzothiazolyl), benzimidazolyl (e.g., benzimidazol-1-yl, benzimidazol-2-yl, benzimidazol-5-yl), benzotriazolyl (e.g., 1H-1,2,3-benzotriazol-5-yl), indolyl (e.g., indol-1-yl, indol-2-yl, indol-3-yl, indol-5-yl), indazolyl (e.g., 1H-indazol-3-yl), pyrrolopyrazinyl (e.g., 1H-pyrrolo[2,3-b]pyrazin-2-yl, 1H-pyrrolo[2,3-b]pyrazin-6-yl), imidazopyridyl (e.g., 1H-imidazo[4,5-b]pyridin-2-yl, 1H-imidazo[4,5-c]pyridin-2-yl, 2H-imidazo[1,2-a]pyridin-3-yl), imidazopyrazinyl (e.g., 1H-imidazo[4,5-b]pyrazin-2-yl), pyrazolopyridyl (e.g., 1H-pyrazolo[4,3-c]pyridin-3-yl), pyrazolothienyl (e.g., 2H-pyrazolo[3,4-b]thiophen-2-yl), pyrazolotriazinyl (e.g., pyrazolo[5,1-c][1,2,4]triazin-3-yl) and the like; and the like.

Examples of the “non-aromatic cyclic group” include a non-aromatic cyclic hydrocarbon group and a non-aromatic heterocyclic group.

Examples of the “non-aromatic cyclic hydrocarbon group” include C₃₋₁₀ cycloalkyl, C₃₋₁₀ cycloalkenyl and C₄₋₁₀ cycloalkadienyl, each of which is optionally condensed with a benzene ring, and the like.

Examples of the “non-aromatic heterocyclic group” include a 4- to 7-membered (preferably 5- or 6-membered) monocyclic non-aromatic heterocyclic group containing, as a ring-constituting atom besides carbon atoms, 1 to 4 heteroatoms selected from an oxygen atom, a sulfur atom and a nitrogen atom, and a fused non-aromatic heterocyclic group. Examples of the fused non-aromatic heterocyclic group include a group derived from a fused ring wherein a ring corresponding to such 4- to 7-membered monocyclic non-aromatic heterocyclic group, and 1 or 2 rings selected from a 5- or 6-membered heterocycle containing 1 or 2 nitrogen atoms, a 5-membered heterocycle containing one sulfur atom and a benzene ring are condensed, and the like.

Examples of the “non-aromatic heterocyclic group” include 4- to 7-membered (preferably 5- or 6-membered) monocyclic non-aromatic heterocyclic groups such as pyrrolidinyl (e.g., 1-pyrrolidinyl, 2-pyrrolidinyl), piperidinyl (e.g., piperidino, 2-piperidinyl, 3-piperidinyl, 4-piperidinyl), morpholinyl (e.g., morpholino), thiomorpholinyl (e.g., thiomorpholino), piperazinyl (e.g., 1-piperazinyl, 2-piperazinyl, 3-piperazinyl), hexamethyleniminyl (e.g., hexamethylenimin-1-yl), oxazolidinyl (e.g., oxazolidin-2-yl), thiazolidinyl (e.g., thiazolidin-2-yl), imidazolidinyl (e.g., imidazolidin-2-yl, imidazolidin-3-yl), oxazolinyl (e.g., oxazolin-2-yl), thiazolinyl (e.g., thiazolin-2-yl), imidazolinyl (e.g., imidazolin-2-yl, imidazolin-3-yl), dioxolyl (e.g., 1,3-dioxol-4-yl), dioxolanyl (e.g., 1,3-dioxolan-4-yl), dihydrooxadiazolyl (e.g., 4,5-dihydro-1,2,4-oxadiazol-3-yl), 2-thioxo-1,3-oxazolidin-5-yl, pyranyl (e.g., 4-pyranyl), tetrahydropyranyl (e.g., 2-tetrahydropyranyl, 3-tetrahydropyranyl, 4-tetrahydropyranyl), thiopyranyl (e.g., 4-thiopyranyl), tetrahydrothiopyranyl (e.g., 2-tetrahydrothiopyranyl, 3-tetrahydrothiopyranyl, 4-tetrahydrothiopyranyl), 1-oxidotetrahydrothiopyranyl (e.g., 1-oxidotetrahydrothiopyran-4-yl), 1,1-dioxidotetrahydrothiopyranyl (e.g., 1,1-dioxidotetrahydrothiopyran-4-yl), tetrahydrofuryl (e.g., tetrahydrofuran-3-yl, tetrahydrofuran-2-yl), pyrazolidinyl (e.g., pyrazolidin-1-yl, pyrazolidin-3-yl), pyrazolinyl (e.g., pyrazolin-1-yl), tetrahydropyrimidinyl (e.g., tetrahydropyrimidin-1-yl), dihydrotriazolyl (e.g., 2,3-dihydro-1H-1,2,3-triazol-1-yl), tetrahydrotriazolyl (e.g., 2,3,4,5-tetrahydro-1H-1,2,3-triazol-1-yl) and the like; fused non-aromatic heterocyclic groups such as dihydroindolyl (e.g., 2,3-dihydro-1H-indol-1-yl), dihydroisoindolyl (e.g., 1,3-dihydro-2H-isoindol-2-yl), dihydrobenzofuranyl (e.g., 2,3-dihydrobenzofuran-5-yl), dihydrobenzodioxinyl (e.g., 2,3-dihydro-1,4-benzodioxinyl), dihydrobenzodioxepinyl (e.g., 3,4-dihydro-2H-1,5-benzodioxepinyl), tetrahydrobenzofuranyl (e.g., 4,5,6,7-tetrahydrobenzofuran-3-yl), chromenyl (e.g., 4H-chromen-2-yl, 2H-chromen-3-yl), dihydroquinolinyl (e.g., 1,2-dihydroquinolin-4-yl), tetrahydroquinolinyl (e.g., 1,2,3,4-tetrahydroquinolin-4-yl), dihydroisoquinolinyl (e.g., 1,2-dihydroisoquinolin-4-yl), tetrahydroisoquinolinyl (e.g., 1,2,3,4-tetrahydroisoquinolin-4-yl), dihydrophthalazinyl (e.g., 1,4-dihydrophthalazin-4-yl) and the like; and the like.

The “cyclic group” optionally has substituent(s) (e.g., 1 to 5, preferably 1 to 3 substituents) at substitutable position(s). When the number of the substituents is not less than 2, respective substituents may be the same or different. Examples of the substituent include groups exemplified as the substituents which the aforementioned “hydrocarbon group” optionally has, and the like.

Examples of the “heterocyclic group” of the “heterocyclic group optionally having substituent(s)” in the present specification include an aromatic heterocyclic group and a non-aromatic heterocyclic group.

Examples of the “aromatic heterocyclic group” and “non-aromatic heterocyclic group” include those similar to the “aromatic heterocyclic group” and “non-aromatic heterocyclic group” which are exemplified as the “cyclic group” of the “cyclic group optionally having substituent(s)”.

The above-mentioned “heterocyclic group” optionally has substituent(s) (e.g., 1 to 5, preferably 1 to 3 substituents) at substitutable position(s). When the number of the substituents is not less than 2, respective substituents may be the same or different. Examples of the substituent include groups exemplified as the substituents which the aforementioned “hydrocarbon group” optionally has, and the like.

Examples of the “hydroxy optionally having a substituent” in the present specification include (1) hydroxy, (2) hydroxy having, instead of a hydrogen atom of hydroxy, for example, a substituent selected from the aforementioned “hydrocarbon group optionally having substituent(s)”, the aforementioned “heterocyclic group optionally having substituent(s)”, the groups exemplified as the substituents which the aforementioned “hydrocarbon group optionally having substituent(s)” optionally has, and the like, and the like.

Specific examples of the “hydroxy optionally having a substituent” include (1) hydroxy, (2) hydroxy optionally having a substituent selected from C₁₋₁₀ alkyl optionally having substituent(s), C₂₋₁₀ alkenyl optionally having substituent(s), C₃₋₁₀ cycloalkyl optionally having substituent(s), C₃₋₁₀ cycloalkenyl optionally having substituent(s), C₆₋₁₄ aryl optionally having substituent(s), C₇₋₁₃ aralkyl optionally having substituent(s), C₈₋₁₃ arylalkenyl optionally having substituent(s), a heterocyclic group optionally having substituent(s), acyl and the like, and the like.

The aforementioned C₁₋₁₀ alkyl, C₂₋₁₀ alkenyl, C₃₋₁₀ cycloalkyl, C₃₋₁₀ cycloalkenyl, C₆₋₁₄ aryl, C₇₋₁₃ aralkyl and C₈₋₁₃ arylalkenyl optionally have substituent(s) (preferably 1 to 3 substituents) at substitutable position(s). Examples of the substituent include groups exemplified as the substituents which the aforementioned “hydrocarbon group” optionally has, and the like. When the number of the substituents is not less than 2, respective substituents may be the same or different.

Examples of the “amino optionally having substituent(s)” in the present specification include (1) amino, (2) amino having, instead of hydrogen atom(s) of amino, for example, 1 or 2 substituents selected from the aforementioned “hydrocarbon group optionally having substituent(s)”, the aforementioned “heterocyclic group optionally having substituent(s)”, the groups exemplified as the substituents which the aforementioned “hydrocarbon group optionally having substituent(s)” optionally has, and the like, and the like.

Specific examples of the “amino optionally having substituent(s)” include (1) amino, (2) amino optionally having 1 or 2 substituents selected from C₁₋₁₀ alkyl optionally having substituent(s), C₂₋₁₀ alkenyl optionally having substituent(s), C₃₋₁₀ cycloalkyl optionally having substituent(s), C₃₋₁₀ cycloalkenyl optionally having substituent(s), C₆₋₁₄ aryl optionally having substituent(s), C₇₋₁₃ aralkyl optionally having substituent(s), C₈₋₁₃ arylalkenyl optionally having substituent(s), a heterocyclic group optionally having substituent(s), acyl and the like, and the like.

The aforementioned C₁₋₁₀ alkyl, C₂₋₁₀ alkenyl, C₃₋₁₀ cycloalkyl, C₃₋₁₀ cycloalkenyl, C₆₋₁₄ aryl, C₇₋₁₃ aralkyl and C₈₋₁₃ arylalkenyl optionally have substituent(s) (preferably 1 to 3 substituents) at substitutable position(s). Examples of the substituent include groups exemplified as the substituents which the aforementioned “hydrocarbon group” optionally has, and the like. When the number of the substituents is not less than 2, respective substituents may be the same or different.

Examples of the “mercapto optionally having a substituent” in the present specification include (1) mercapto, (2) mercapto having, instead of a hydrogen atom of mercapto, for example, a substituent selected from the aforementioned “hydrocarbon group optionally having substituent(s)”, the aforementioned “heterocyclic group optionally having substituent(s)”, the groups exemplified as the substituents which the aforementioned “hydrocarbon group optionally having substituent(s)” optionally has, and the like, and the like.

Specific examples of the “mercapto optionally having a substituent” include (1) mercapto, (2) mercapto optionally having a substituent selected from C₁₋₁₀ alkyl optionally having substituent(s), C₂₋₁₀ alkenyl optionally having substituent(s), C₃₋₁₀ cycloalkyl optionally having substituent(s), C₃₋₁₀ cycloalkenyl optionally having substituent(s), C₆₋₁₄ aryl optionally having substituent(s), C₇₋₁₃ aralkyl optionally having substituent(s), C₈₋₁₃ arylalkenyl optionally having substituent(s), a heterocyclic group optionally having substituent(s), acyl and the like, and the like.

The aforementioned C₁₋₁₀ alkyl, C₂₋₁₀ alkenyl, C₃₋₁₀ cycloalkyl, C₃₋₁₀ cycloalkenyl, C₆₋₁₄ aryl, C₇₋₁₃ aralkyl and CB-13 arylalkenyl optionally have substituent(s) (preferably 1 to 3 substituents) at substitutable position(s). Examples of the substituent include groups exemplified as the substituents which the aforementioned “hydrocarbon group” optionally has, and the like. When the number of the substituents is not less than 2, respective substituents may be the same or different.

Examples of the “acyl” in the present specification include a group represented by the formula: —COR^{A}, —CO—OR^{A}, —SO₂R^{A}, —SOR^{A}, —CO—NR^{A}′R^{B}′ or —CS—NR^{A}′R^{B}′ [wherein R^{A} is a hydrogen atom, a hydrocarbon group optionally having substituent(s) or a heterocyclic group optionally having substituent(s), and R^{A}′ and R^{B}′ are the same or different and each is a hydrogen atom, a hydrocarbon group optionally having substituent(s) or a heterocyclic group optionally having substituent(s), or R^{A}′ and R^{B}′ optionally form, together with the adjacent nitrogen atom, a nitrogen-containing heterocycle optionally having substituent(s)] and the like.

Examples of the “nitrogen-containing heterocycle” of the “nitrogen-containing heterocycle optionally having substituent(s)” formed by R^{A}′ and R^{B}′ together with the adjacent nitrogen atom include a 5- to 7-membered nitrogen-containing heterocycle containing, as a ring-constituting atom besides carbon atoms, at least one nitrogen atom and optionally further containing one or two heteroatoms selected from an oxygen atom, a sulfur atom and a nitrogen atom. Specific examples of the nitrogen-containing heterocycle include pyrrolidine, imidazolidine, pyrazolidine, piperidine, piperazine, morpholine, thiomorpholine and the like.

The nitrogen-containing heterocycle optionally has substituent(s) (preferably 1 to 3, more preferably 1 or 2 substituents) at substitutable position(s). Examples of the substituent include groups exemplified as the substituents which the aforementioned “hydrocarbon group” optionally has, and the like. When the number of the substituents is not less than 2, respective substituents may be the same or different.

Preferable examples of the “acyl” include

(1) formyl;
(2) carboxy;
(3) carbamoyl;
(4) C₁₋₆ alkyl-carbonyl;
(5) C₁₋₆ alkoxy-carbonyl optionally having 1 to 3 substituents selected from carboxy, carbamoyl, thiocarbamoyl, C₁₋₆ alkoxy-carbonyl and C₁₋₆ alkyl-carbonyloxy (e.g., methoxycarbonyl, ethoxycarbonyl, propoxycarbonyl, tert-butoxycarbonyl; carboxymethoxycarbonyl, carboxyethoxycarbonyl, carboxybutoxycarbonyl; carbamoylmethoxycarbonyl; thiocarbamoylmethoxycarbonyl; ethoxycarbonylmethoxycarbonyl, ethoxycarbonylethoxycarbonyl, methoxycarbonylbutoxycarbonyl, ethoxycarbonylbutoxycarbonyl; tert-butylcarbonyloxymethoxycarbonyl);
(6) C₃₋₁₀ cycloalkyl-carbonyl (e.g., cyclopentylcarbonyl, cyclohexylcarbonyl);
(7) C₆₋₁₄ aryl-carbonyl (e.g., benzoyl, 1-naphthoyl, 2-naphthoyl) optionally having 1 to 3 substituents selected from a halogen atom, cyano, C₁₋₆ alkyl optionally substituted by 1 to 3 halogen atoms, C₁₋₆ alkoxy, carboxy, C₁₋₆ alkoxy-carbonyl, an aromatic heterocyclic group (e.g., tetrazolyl, oxadiazolyl), a non-aromatic heterocyclic group (e.g., oxooxadiazolyl) and carbamoyl;
(8) C₆₋₁₄ aryloxy-carbonyl (e.g., phenyloxycarbonyl, naphthyloxycarbonyl) optionally having 1 to 3 substituents selected from carboxy, C₁₋₆ alkoxy-carbonyl and carbamoyl;
(9) C₇₋₁₃ aralkyloxy-carbonyl optionally having 1 to 3 substituents selected from carboxy, carbamoyl, thiocarbamoyl, C₁₋₆ alkoxy-carbonyl, a halogen atom, cyano, nitro, C₁₋₆ alkoxy, C₁₋₆ alkylsulfonyl and C₁₋₆ alkyl (e.g., benzyloxycarbonyl, phenethyloxycarbonyl; carboxybenzyloxycarbonyl; methoxycarbonylbenzyloxycarbonyl; biphenylylmethoxycarbonyl);
(10) carbamoyl mono- or di-substituted by C₁₋₆ alkyl optionally having 1 to 3 substituents selected from a halogen atom and C₁₋₆ alkoxy (e.g., methylcarbamoyl, ethylcarbamoyl, dimethylcarbamoyl, diethylcarbamoyl, ethylmethylcarbamoyl, propylcarbamoyl, isopropylcarbamoyl, butylcarbamoyl, isobutylcarbamoyl, trifluoroethylcarbamoyl, N-methoxyethyl-N-methylcarbamoyl);
(11) C₁₋₆ alkylsulfonyl optionally having 1 to 3 substituents selected from carboxy, carbamoyl and C₁₋₆ alkoxy-carbonyl (e.g., methylsulfonyl, carboxymethylsulfonyl);
(12) C₁₋₆ alkylsulfinyl (e.g., methylsulfinyl);
(13) thiocarbamoyl;
(14) C₇₋₁₃ aralkyl-carbonyl (e.g., benzylcarbonyl, phenethylcarbonyl);
(15) aromatic heterocyclyl (e.g., furyl, thienyl, oxazolyl, thiazolyl, isoxazolyl, isothiazolyl, pyrazolyl, pyridyl, pyrazinyl, benzofuryl, benzothienyl, quinoxalinyl)-carbonyl (e.g., furylcarbonyl, thienylcarbonyl, thiazolylcarbonyl, pyrazolylcarbonyl, pyridylcarbonyl, pyrazinylcarbonyl, benzofurylcarbonyl, benzothienylcarbonyl, quinoxalinylcarbonyl) optionally having 1 to 3 substituents selected from C₁₋₆ alkyl, C₆₋₁₄ aryl, C₇₋₁₃ aralkyl, C₁₋₆ alkoxy, carboxy, C₁₋₆ alkoxy-carbonyl and carbamoyl; and the like.

Each symbol in the formula (I) is described in detail in the following.

R¹ is a substituent.

Examples of the “substituent” for R¹ include a halogen atom, a hydrocarbon group optionally having substituent(s), a heterocyclic group optionally having substituent(s), hydroxy optionally having a substituent, amino optionally having substituent(s), acyl and the like.

R¹ is preferably a halogen atom, a hydrocarbon group optionally having substituent(s), hydroxy optionally having a substituent, amino optionally having substituent(s) or the like, more preferably a hydrocarbon group optionally having substituent(s), further more preferably C₁₋₆ alkyl optionally having substituent(s), C₇₋₁₃ aralkyl optionally having substituent(s) or the like, particularly preferably

(a) C₁₋₆ alkyl substituted by hydroxy optionally having a substituent (e.g., a hydrocarbon group optionally having substituent(s), a heterocyclic group optionally having substituent(s), heterocyclylcarbonyl optionally having substituent(s)),
(b) C₁₋₆ alkyl substituted by a heterocyclic group optionally having substituent(s) or amino optionally having substituent(s),
(c) C₇₋₁₃ aralkyl optionally having substituent(s) (e.g., a halogen atom, C₁₋₆ alkyl optionally having substituent(s), cyano, hydroxy, C₁₋₆ alkoxy optionally having substituent(s), a heterocyclic group optionally having substituent(s)), or the like.

More preferably, R¹ is

(a) C₁₋₆ alkyl substituted by hydroxy optionally having a substituent (e.g., a fused aromatic heterocyclic group such as benzofuranyl (e.g., 5-benzofuranyl, 6-benzofuranyl), benzothienyl (e.g., 5-benzothienyl, 6-benzothienyl), benzoxazolyl (e.g., 5-benzoxazolyl, 6-benzoxazolyl), benzisoxazolyl (e.g., 5-benzisoxazolyl, 6-benzisoxazolyl), benzothiazolyl (e.g., 5-benzothiazolyl, 6-benzothiazolyl), benzimidazolyl (e.g., benzimidazol-5-yl), benzotriazolyl (e.g., 1H-1,2,3-benzotriazol-5-yl), indolyl (e.g., indol-5-yl, indol-6-yl), indazolyl (e.g., 1H-indazol-5-yl, 1H-indazol-6-yl), pyrrolopyrazinyl (e.g., 1H-pyrrolo[2,3-b]pyrazin-2-yl, 1H-pyrrolo[2,3-b]pyrazin-6-yl), imidazopyridyl (e.g., 1H-imidazo[4,5-b]pyridin-5-yl, 1H-imidazo[4,5-c]pyridin-5-yl, 2H-imidazo[1,2-a]pyridin-5-yl), imidazopyrazinyl (e.g., 1H-imidazo[4,5-b]pyrazin-5-yl), pyrazolopyridyl (e.g., 1H-pyrazolo[4,3-c]pyridin-5-yl), pyrazolothienyl (e.g., 2H-pyrazolo[3,4-b]thiophen-2-yl) and the like),
(b) C₁₋₆ alkyl substituted by phenylamino optionally having substituent(s) (e.g., a halogen atom, C₁₋₆ alkyl optionally having substituent(s), C₁₋₆ alkoxy optionally having substituent(s)),
(c) C₇₋₁₃ aralkyl optionally having substituent(s) (e.g., a halogen atom, C₁₋₆ alkyl optionally having substituent(s), C₁₋₆ alkoxy optionally having substituent(s), a monocyclic aromatic heterocyclic group optionally having substituent(s)), or the like.

Preferable embodiments of R¹ include

(1) C₇₋₁₃ aralkyl (e.g., benzyl, phenethyl, phenylpropyl) optionally having 1 to 3 substituents selected from
(i) a halogen atom,
(ii) C₁₋₆ alkyl optionally having 1 to 5 substituents selected from a halogen atom and hydroxy,
(iii) cyano,
(iv) hydroxy,
(v) optionally halogenated C₁₋₆ alkoxy (e.g., trifluoromethoxy), and
(vi) a 5- or 6-membered non-aromatic heterocyclic group (e.g., morpholinyl);
   (2) C₃₋₁₀ cycloalkyl-C₁₋₆ alkyl (e.g., cyclopropylmethyl, cyclohexylmethyl) optionally having one hydroxy;
   (3) C₁₋₆ alkyl optionally having 1 to 5 substituents selected from
(i) a halogen atom,
(ii) hydroxy optionally having a substituent selected from
   (a) C₆₋₁₀ aryl (e.g., phenyl) optionally having 1 to 3 substituents selected from
      A) a halogen atom,
      B) cyano,
      C) C₁₋₆ alkyl optionally having 1 or 2 substituents selected from carboxy, hydroxy, C₁₋₆ alkoxy-carbonyl and mono- or di-C₁₋₆ alkylamino,
      D) optionally halogenated C₁₋₆ alkoxy (e.g., methoxy, trifluoromethoxy, ethoxy, isopropoxy),
      E) C₁₋₄ alkylenedioxy,
      F) carboxy,
      G) C₁₋₆ alkyl-carbonyl,
      H) C₁₋₆ alkoxy-carbonyl,
      I) 5- or 6-membered non-aromatic heterocyclylcarbonyl (e.g., azetidinylcarbonyl),
      J) carbamoyl,
      K) optionally halogenated mono- or di-C₁₋₆ alkyl-carbamoyl,
      L) C₃₋₆ cycloalkyl-carbamoyl (e.g., cyclopropylcarbamoyl),
      M) mono- or di-C₁₋₆ alkylamino,
      O) optionally halogenated C₁₋₆ alkylsulfonyl (e.g., methylsulfonyl, trifluoromethylsulfonyl), and
      P) a 5- or 6-membered heterocyclic group (e.g., imidazolyl, pyrazolyl, triazolyl, oxadiazolyl, pyrrolidinyl, piperazinyl) optionally having 1 or 2 substituents selected from C₁₋₆ alkyl, C₁₋₆ alkyl-carbonyl and oxo,
   (b) C₆₋₁₀ aryl condensed with C₃₋₁₀ cycloalkane (e.g., tetrahydronaphthyl) optionally having 1 or 2 oxo,
   (c) a 5- or 6-membered aromatic heterocyclic group (e.g., pyrazolyl, triazolyl, thienyl, thiazolyl, isoxazolyl, pyridyl, pyrimidinyl; the 5- or 6-membered aromatic heterocyclic group is optionally oxidized) optionally having 1 to 3 substituents selected from a halogen atom, cyano, optionally halogenated C₁₋₆ alkyl, C₁₋₆ alkoxy-carbonyl-C₁₋₆ alkyl, mono- or di-C₁₋₆ alkylamino-C₁₋₆ alkyl (e.g., dimethylaminomethyl), C₆₋₁₀ aryl (e.g., phenyl), C₁₋₆ alkoxy-carbonyl and carboxy,
   (d) a 9- or 10-membered fused heterocyclic group (e.g., benzothiazolyl, dihydrobenzoxazolyl, benzisoxazolyl, dihydrobenzofuranyl, tetrahydroquinolyl, tetrahydroisoquinolyl, chromenyl, thienopyridyl) optionally having 1 to 3 substituents selected from C₁₋₆ alkyl, C₁₋₆ alkoxy and oxo,
   (e) C₇₋₁₃ aralkyl (e.g., benzyl),
   (f) C₃₋₁₀ cycloalkyl-C₁₋₆ alkyl (e.g., cyclopropylmethyl), and
   (g) 5- or 6-membered non-aromatic heterocyclylcarbonyl (e.g., pyrrolidinylcarbonyl),
(iii) C₆₋₁₀ arylthio (e.g., phenylthio),
(iv) amino optionally having 1 or 2 substituents selected from
   (a) C₁₋₆ alkyl,
   (b) C₆₋₁₀ aryl (e.g., phenyl),
   (c) C₃₋₆ cycloalkyl-carbonyl,
   (d) C₆₋₁₀ aryl-carbonyl optionally having 1 to 3 substituents selected from a halogen atom and C₁₋₆ alkoxy,
   (e) C₁₋₆ alkoxy-carbonyl-C₁₋₆ alkyl-carbonyl, and
   (f) carbamoyl-C₁₋₆ alkyl-carbonyl,
(v) a 5- or 6-membered heterocyclic group (e.g., piperidinyl, pyrrolyl, imidazolyl, pyrazolyl, triazolyl, thiazolyl, oxadiazolyl, pyridyl) optionally having 1 to 3 substituents selected from
   (a) C₁₋₆ alkyl optionally having 1 to 5 substituents selected from a halogen atom, hydroxy and C₁₋₆ alkyl-carbonyloxy,
   (b) C₃₋₆ cycloalkyl,
   (c) C₆₋₁₀ aryl (e.g., phenyl),
   (d) C₁₋₆ alkyl-carbonyl, and
   (e) C₁₋₆ alkoxy-carbonyl, and
(vi) a 9- or 10-membered fused heterocyclic group (e.g., indolyl, indazolyl, dihydroindazolyl, tetrahydroindazolyl, benzotriazolyl, benzimidazolyl, dihydrobenzimidazolyl, dihydrobenzoxazolyl, dihydrobenzoxazinyl) optionally having 1 to 3 substituents selected from cyano, C₁₋₆ alkyl, C₃₋₆ cycloalkyl, C₁₋₆ alkoxy-carbonyl and oxo;
   (4) C₃₋₁₀ cycloalkyl optionally condensed with a benzene ring (e.g., indanyl); and the like.

Other preferable embodiments of R¹ include

(1) C₇₋₁₃ aralkyl (e.g., benzyl, phenethyl, phenylpropyl, naphthylmethyl, biphenylylmethyl) optionally having 1 to 3 substituents selected from
(i) a halogen atom,
(ii) C₁₋₆ alkyl optionally having 1 to 5 substituents selected from a halogen atom and hydroxy,
(iii) cyano,
(iv) hydroxy,
(v) optionally halogenated C₁₋₆ alkoxy (e.g., methoxy, trifluoromethoxy),
(vi) C₆₋₁₀ aryloxy (e.g., phenoxy),
(vii) a 5- or 6-membered non-aromatic heterocyclic group (e.g., morpholinyl), and
(viii) a 5- or 6-membered aromatic heterocyclic group (e.g., pyridyl, pyrazolyl, oxadiazolyl) optionally having 1 to 3 substituents selected from C₁₋₆ alkyl and C₁₋₆ alkoxy;
   (2) C₃₋₁₀ cycloalkyl-C₁₋₆ alkyl (e.g., cyclopropylmethyl, cyclohexylmethyl) optionally having one hydroxy;
   (3) C₁₋₆ alkyl optionally having 1 to 5 substituents selected from
(i) a halogen atom,
(ii) hydroxy optionally having a substituent selected from
   (a) C₆₋₁₀ aryl (e.g., phenyl, naphthyl) optionally having 1 to 3 substituents selected from
      A) a halogen atom,
      B) cyano,
      C) C₁₋₆ alkyl optionally having 1 or 2 substituents selected from carboxy, hydroxy, C₁₋₆ alkoxy-carbonyl and mono- or di-C₁₋₆ alkylamino,
      D) optionally halogenated C₁₋₆ alkoxy (e.g., methoxy, trifluoromethoxy, ethoxy, isopropoxy, difluoromethoxy),
      E) C₁₋₄ alkylenedioxy,
      F) carboxy,
      G) C₁₋₆ alkyl-carbonyl,
      H) C₁₋₆ alkoxy-carbonyl,
      I) 5- or 6-membered non-aromatic heterocyclylcarbonyl (e.g., azetidinylcarbonyl),
      J) carbamoyl,
      K) optionally halogenated mono- or di-C₁₋₆ alkyl-carbamoyl,
      L) C₃₋₆ cycloalkyl-carbamoyl (e.g., cyclopropylcarbamoyl),
      M) mono- or di-C₁₋₆ alkylamino,
      O) optionally halogenated C₁₋₆ alkylsulfonyl (e.g., methylsulfonyl, trifluoromethylsulfonyl),
      P) a 5- or 6-membered heterocyclic group (e.g., imidazolyl, pyrazolyl, triazolyl, oxadiazolyl, pyrrolidinyl, piperazinyl, morpholinyl) optionally having 1 or 2 substituents selected from C₁₋₆ alkyl, C₁₋₆ alkyl-carbonyl and oxo, and
      Q) a 9- or 10-membered fused heterocyclic group (e.g., dihydroimidazoimidazolyl) optionally having 1 to 3 substituents selected from C₁₋₆ alkyl and oxo,
   (b) C₆₋₁₀ aryl condensed with C₃₋₁₀ cycloalkane (e.g., tetrahydronaphthyl) optionally having 1 or 2 oxo,
   (c) a 5- or 6-membered aromatic heterocyclic group (e.g., pyrazolyl, triazolyl, thienyl, thiazolyl, isoxazolyl, pyridyl, pyrimidinyl; the 5- or 6-membered aromatic heterocyclic group is optionally oxidized) optionally having 1 to 3 substituents selected from a halogen atom, cyano, optionally halogenated C₁₋₆ alkyl, C₁₋₆ alkoxy-carbonyl-C₁₋₆ alkyl, mono- or di-C₁₋₆ alkylamino-C₁₋₆ alkyl (e.g., dimethylaminomethyl), C₆₋₁₀ aryl (e.g., phenyl), C₁₋₆ alkoxy-carbonyl and carboxy,
   (d) a 9- or 10-membered fused heterocyclic group (e.g., benzothiazolyl, benzimidazolyl, benzothienyl, benzoxazolyl, benzotriazolyl, indolyl, indazolyl, imidazopyridyl, pyrazolopyridyl, dihydrobenzoxazolyl, benzisoxazolyl, benzisothiazolyl, benzofuranyl, dihydrobenzofuranyl, tetrahydroquinolyl, tetrahydroisoquinolyl, chromenyl, thienopyridyl, pyrrolopyrazinyl, imidazopyrazinyl, pyrazolothienyl, dihydrofuropyridyl) optionally having 1 to 3 substituents selected from C₁₋₆ alkyl, C₁₋₆ alkoxy, C₁₋₆ alkoxy-carbonyl, C₃₋₁₀ cycloalkyl, a halogen atom and oxo,
   (e) C₇₋₁₃ aralkyl (e.g., benzyl),
   (f) C₃₋₁₀ cycloalkyl-C₁₋₆ alkyl (e.g., cyclopropylmethyl),
   (g) 5- or 6-membered non-aromatic heterocyclylcarbonyl (e.g., pyrrolidinylcarbonyl), and
   (h) C₆₋₁₀ aryl-carbamoyl (e.g., phenylcarbamoyl),
(iii) C₆₋₁₀ arylthio (e.g., phenylthio),
(iv) C₆₋₁₀ arylsulfinyl (e.g., phenylsulfinyl),
(v) optionally halogenated C₆₋₁₀ arylsulfonyl (e.g., phenylsulfonyl, fluorophenylsulfonyl),
(vi) amino optionally having 1 or 2 substituents selected from
   (a) C₁₋₆ alkyl,
   (b) C₆₋₁₀ aryl (e.g., phenyl) optionally having 1 to 3 substituents selected from
      A) a halogen atom,
      B) optionally halogenated C₁₋₆ alkyl (e.g., methyl, ethyl, isopropyl, trifluoromethyl),
      C) optionally halogenated C₁₋₆ alkoxy (e.g., methoxy, trifluoromethoxy, difluoromethoxy),
      D) cyano,
      E) nitro,
      F) carboxy,
      G) C₁₋₆ alkyl-carbonyl,
      H) C₁₋₆ alkoxy-carbonyl,
      I) C₁₋₄ alkylenedioxy, and
      J) a 5- or 6-membered heterocyclic group (e.g., pyrazolyl, piperidinyl, dihydropyridyl) optionally having 1 or 2 oxo,
   (c) C₃₋₆ cycloalkyl optionally condensed with a benzene ring (e.g., cyclopropyl, cyclohexyl, indanyl),
   (d) C₇₋₁₃ aralkyl (e.g., benzyl),
   (e) C₁₋₆ alkyl-carbonyl,
   (f) C₃₋₆ cycloalkyl-carbonyl,
   (g) C₆₋₁₀ aryl-carbonyl optionally having 1 to 3 substituents selected from a halogen atom and C₁₋₆ alkoxy,
   (h) C₁₋₆ alkoxy-carbonyl-C₁₋₆ alkyl-carbonyl,
   (i) carbamoyl-C₁₋₆ alkyl-carbonyl,
   (j) a 5- or 6-membered heterocyclic group (e.g., pyridyl), and
   (k) a 9- or 10-membered fused heterocyclic group (e.g., benzothiazolyl, benzimidazolyl, benzothienyl, benzoxazolyl, benzotriazolyl, indolyl, indazolyl, imidazopyridyl, pyrazolopyridyl, dihydrobenzoxazolyl, benzisoxazolyl, benzisothiazolyl, benzofuranyl, dihydrobenzofuranyl, tetrahydroquinolyl, tetrahydroisoquinolyl, chromenyl, thienopyridyl, pyrrolopyrazinyl, imidazopyrazinyl, pyrazolothienyl, dihydrofuropyridyl) optionally having 1 to 3 substituents selected from C₁₋₆ alkyl and oxo,
(vii) a 5- or 6-membered heterocyclic group (e.g., piperidinyl, pyrrolyl, imidazolyl, pyrazolyl, triazolyl, thiazolyl, oxadiazolyl, pyridyl) optionally having 1 to 3 substituents selected from
   (a) C₁₋₆ alkyl optionally having 1 to 5 substituents selected from a halogen atom, hydroxy and C₁₋₆ alkyl-carbonyloxy,
   (b) C₃₋₆ cycloalkyl,
   (c) C₆₋₁₀ aryl (e.g., phenyl),
   (d) C₁₋₆ alkyl-carbonyl, and
   (e) C₁₋₆ alkoxy-carbonyl,
(viii) a 9- or 10-membered fused heterocyclic group (e.g., indolyl, dihydroisoindolyl, indazolyl, dihydroindazolyl, tetrahydroindazolyl, benzotriazolyl, benzimidazolyl, dihydrobenzimidazolyl, dihydrobenzoxazolyl, dihydrobenzoxazinyl, tetrahydroquinolyl, tetrahydroisoquinolyl) optionally having 1 to 3 substituents selected from cyano, C₁₋₆ alkyl, C₃₋₆ cycloalkyl, C₁₋₆ alkoxy-carbonyl and oxo,
(ix) carbamoyl optionally having 1 or 2 substituents selected from C₁₋₆ alkyl and C₆₋₁₀ aryl,
(x) 5- or 6-membered heterocyclylthio (e.g., thiazolylthio, thiadiazolylthio, triazolylthio) optionally having C₁₋₆ alkyl optionally having 1 to 3 substituents selected from hydroxy and C₁₋₆ alkyl-carbonyloxy, and
(xi) a 9- or 10-membered fused heterocyclylthio (e.g., benzothiazolylthio, benzimidazolylthio, thiazolopyridylthio);
   (4) C₃₋₁₀ cycloalkyl optionally condensed with a benzene ring (e.g., indanyl); and the like.

Still other preferable embodiments of R¹ include

(1) C₇₋₁₃ aralkyl (e.g., benzyl, phenethyl, phenylpropyl, naphthylmethyl, biphenylylmethyl) optionally having 1 to 3 substituents selected from
(i) a halogen atom,
(ii) C₁₋₆ alkyl optionally having 1 to 5 substituents selected from a halogen atom and hydroxy,
(iii) cyano,
(iv) hydroxy,
(v) optionally halogenated C₁₋₆ alkoxy (e.g., methoxy, trifluoromethoxy),
(vi) C₆₋₁₀ aryloxy (e.g., phenoxy),
(vii) a 5- or 6-membered non-aromatic heterocyclic group (e.g., morpholinyl), and
(viii) a 5- or 6-membered aromatic heterocyclic group (e.g., pyridyl, pyrazolyl, oxadiazolyl) optionally having 1 to 3 substituents selected from C₁₋₆ alkyl and C₁₋₆ alkoxy;
   (2) C₃₋₁₀ cycloalkyl-C₁₋₆ alkyl (e.g., cyclopropylmethyl, cyclohexylmethyl) optionally having one hydroxy;
   (3) C₁₋₆ alkyl optionally having 1 to 5 substituents selected from
(i) a halogen atom,
(ii) hydroxy optionally having a substituent selected from
   (a) C₆₋₁₀ aryl (e.g., phenyl, naphthyl) optionally having 1 to 3 substituents selected from
      A) a halogen atom,
      B) cyano,
      C) C₁₋₆ alkyl optionally having 1 to 3 substituents selected from a halogen atom, carboxy, hydroxy, C₁₋₆ alkoxy-carbonyl and mono- or di-C₁₋₆ alkylamino,
      D) C₁₋₆ alkoxy optionally having 1 to 3 substituents selected from a halogen atom and C₁₋₆ alkoxy,
      E) C₁₋₄ alkylenedioxy,
      F) carboxy,
      G) C₁₋₆ alkyl-carbonyl,
      H) C₁₋₆ alkoxy-carbonyl,
      I) 5- or 6-membered non-aromatic heterocyclylcarbonyl (e.g., azetidinylcarbonyl),
      J) carbamoyl,
      K) optionally halogenated mono- or di-C₁₋₆ alkyl-carbamoyl,
      L) C₃₋₆ cycloalkyl-carbamoyl (e.g., cyclopropylcarbamoyl),
      M) mono- or di-C₁₋₆ alkylamino,
      O) optionally halogenated C₁₋₆ alkylsulfonyl (e.g., methylsulfonyl, trifluoromethylsulfonyl),
      P) a 5- or 6-membered heterocyclic group (e.g., imidazolyl, pyrazolyl, triazolyl, oxadiazolyl, pyrrolidinyl, piperazinyl, morpholinyl) optionally having 1 or 2 substituents selected from C₁₋₆ alkyl, C₁₋₆ alkyl-carbonyl and oxo, and
      Q) a 9- or 10-membered fused heterocyclic group (e.g., dihydroimidazoimidazolyl) optionally having 1 to 3 substituents selected from C₁₋₆ alkyl and oxo,
   (b) C₆₋₁₀ aryl condensed with C₃₋₁₀ cycloalkane (e.g., tetrahydronaphthyl) optionally having 1 or 2 oxo,
   (c) a 5- or 6-membered aromatic heterocyclic group (e.g., pyrazolyl, triazolyl, thienyl, thiazolyl, isoxazolyl, pyridyl, pyrimidinyl; the 5- or 6-membered aromatic heterocyclic group is optionally oxidized) optionally having 1 to 3 substituents selected from a halogen atom, cyano, optionally halogenated C₁₋₆ alkyl, C₁₋₆ alkoxy-carbonyl-C₁₋₆ alkyl, mono- or di-C₁₋₆ alkylamino-C₁₋₆ alkyl (e.g., dimethylaminomethyl), C₆₋₁₀ aryl (e.g., phenyl), C₁₋₆ alkoxy-carbonyl and carboxy,
   (d) a 9- or 10-membered fused heterocyclic group (e.g., benzothiazolyl, benzimidazolyl, benzothienyl, benzoxazolyl, benzotriazolyl, indolyl, indazolyl, imidazopyridyl, pyrazolopyridyl, dihydrobenzoxazolyl, benzisoxazolyl, benzisothiazolyl, benzofuranyl, dihydrobenzofuranyl, tetrahydroquinolyl, tetrahydroisoquinolyl, chromenyl, thienopyridyl, pyrrolopyrazinyl, imidazopyrazinyl, pyrazolothienyl, dihydrofuropyridyl, dihydrobenzoxazinyl) optionally having 1 to 3 substituents selected from
      A) C₁₋₆ alkyl optionally having 1 to 3 substituents selected from C₁₋₆ alkoxy and C₁₋₆ alkoxy-carbonyl,
      B) C₁₋₆ alkoxy,
      C) C₁₋₆ alkoxy-carbonyl,
      D) C₃₋₁₀ cycloalkyl,
      E) a halogen atom, and
      F) oxo,
   (e) C₇₋₁₃ aralkyl (e.g., benzyl),
   (f) C₃₋₁₀ cycloalkyl-C₁₋₆ alkyl (e.g., cyclopropylmethyl),
   (g) 5- or 6-membered non-aromatic heterocyclylcarbonyl (e.g., pyrrolidinylcarbonyl),
   (h) C₆₋₁₀ aryl-carbamoyl (e.g., phenylcarbamoyl), and
   (i) C₃₋₆ cycloalkyl optionally condensed with a benzene ring (e.g., indanyl),
(iii) C₆₋₁₀ arylthio (e.g., phenylthio),
(iv) C₆₋₁₀ arylsulfinyl (e.g., phenylsulfinyl),
(v) optionally halogenated C₆₋₁₀ arylsulfonyl (e.g., phenylsulfonyl, fluorophenylsulfonyl),
(vi) amino optionally having 1 or 2 substituents selected from
   (a) C₁₋₆ alkyl,
   (b) C₆₋₁₀ aryl (e.g., phenyl) optionally having 1 to 3 substituents selected from
      A) a halogen atom,
      B) optionally halogenated C₁₋₆ alkyl (e.g., methyl, ethyl, isopropyl, trifluoromethyl),
      C) optionally halogenated C₁₋₆ alkoxy (e.g., methoxy, trifluoromethoxy, difluoromethoxy),
      D) cyano,
      E) nitro,
      F) carboxy,
      G) C₁₋₆ alkyl-carbonyl,
      H) C₁₋₆ alkoxy-carbonyl,
      I) C₁₋₄ alkylenedioxy, and
      J) a 5- or 6-membered heterocyclic group (e.g., pyrazolyl, piperidinyl, dihydropyridyl) optionally having 1 or 2 oxo,
   (c) C₃₋₆ cycloalkyl optionally condensed with a benzene ring (e.g., cyclopropyl, cyclohexyl, indanyl),
   (d) C₇₋₁₃ aralkyl (e.g., benzyl),
   (e) C₁₋₆ alkyl-carbonyl,
   (f) C₃₋₆ cycloalkyl-carbonyl,
   (g) C₆₋₁₀ aryl-carbonyl optionally having 1 to 3 substituents selected from a halogen atom and C₁₋₆ alkoxy,
   (h) C₁₋₆ alkoxy-carbonyl-C₁₋₆ alkyl-carbonyl,
   (i) carbamoyl-C₁₋₆ alkyl-carbonyl,
   (j) a 5- or 6-membered heterocyclic group (e.g., pyridyl), and
   (k) a 9- or 10-membered fused heterocyclic group (e.g., benzothiazolyl, benzimidazolyl, benzothienyl, benzoxazolyl, benzotriazolyl, indolyl, indazolyl, imidazopyridyl, pyrazolopyridyl, dihydrobenzoxazolyl, benzisoxazolyl, benzisothiazolyl, benzofuranyl, dihydrobenzofuranyl, tetrahydroquinolyl, tetrahydroisoquinolyl, chromenyl, thienopyridyl, pyrrolopyrazinyl, imidazopyrazinyl, pyrazolothienyl, dihydrofuropyridyl, dihydrobenzoxazinyl) optionally having 1 to 3 substituents selected from C₁₋₆ alkyl and oxo,
(vii) a 5- or 6-membered heterocyclic group (e.g., piperidinyl, pyrrolyl, imidazolyl, pyrazolyl, triazolyl, thiazolyl, oxadiazolyl, pyridyl, tetrazolyl) optionally having 1 to 3 substituents selected from
   (a) C₁₋₆ alkyl optionally having 1 to 5 substituents selected from a halogen atom, hydroxy and C₁₋₆ alkyl-carbonyloxy,
   (b) C₃₋₆ cycloalkyl,
   (c) C₆₋₁₀ aryl (e.g., phenyl),
   (d) C₁₋₆ alkyl-carbonyl, and
   (e) C₁₋₆ alkoxy-carbonyl,
(viii) a 9- or 10-membered fused heterocyclic group (e.g., indolyl, dihydroisoindolyl, indazolyl, dihydroindazolyl, tetrahydroindazolyl, benzotriazolyl, benzimidazolyl, dihydrobenzimidazolyl, dihydrobenzoxazolyl, dihydrobenzoxazinyl, tetrahydroquinolyl, tetrahydroisoquinolyl) optionally having 1 to 3 substituents selected from cyano, C₁₋₆ alkyl, C₃₋₆ cycloalkyl, C₁₋₆ alkoxy-carbonyl and oxo,
(ix) carbamoyl optionally having 1 or 2 substituents selected from C₁₋₆ alkyl and C₆₋₁₀ aryl,
(x) 5- or 6-membered heterocyclylthio (e.g., thiazolylthio, thiadiazolylthio, triazolylthio) optionally having C₁₋₆ alkyl optionally having 1 to 3 substituents selected from hydroxy and C₁₋₆ alkyl-carbonyloxy, and
(xi) 9- or 10-membered fused heterocyclylthio (e.g., benzothiazolylthio, benzimidazolylthio, thiazolopyridylthio);
   (4) C₃₋₁₀ cycloalkyl optionally condensed with a benzene ring (e.g., indanyl); and the like.

R² is a cyclic group optionally having substituent(s), C₁₋₁₀ alkyl optionally having substituent(s), C₂₋₁₀ alkenyl optionally having substituent(s) or C₂₋₁₀ alkynyl optionally having substituent(s).

The aforementioned C₁₋₁₀ alkyl, C₂₋₁₀ alkenyl and C₂₋₁₀ alkynyl optionally have substituent(s) (e.g., 1 to 5, preferably 1 to 3 substituents) at substitutable position(s). Examples of the substituent include groups exemplified as the substituents which the aforementioned “hydrocarbon group” optionally has, and the like. When the number of the substituents is not less than 2, respective substituents may be the same or different.

R² is preferably C₆₋₁₄ aryl optionally having substituent(s), C₃₋₁₀ cycloalkyl optionally having substituent(s) or the like.

R² is more preferably optionally halogenated C₆₋₁₀ aryl (e.g., phenyl), C₃₋₆ cycloalkyl (e.g., cyclopropyl, cyclohexyl) or the like.

R² is particularly preferably optionally halogenated C₆₋₁₀ aryl (e.g., phenyl) or the like.

R³ is a hydrogen atom, a halogen atom, C₁₋₆ alkyl or C₁₋₆ alkoxy.

R³ is preferably a hydrogen atom, a halogen atom, C₁₋₃ alkyl (e.g., methyl, ethyl, propyl, isopropyl), C₁₋₃ alkoxy (e.g., methoxy, ethoxy, propoxy, isopropoxy) or the like, more preferably a hydrogen atom or the like.

X is bond or spacer having 1 to 6 atoms in the main chain.

The “main chain” of the “spacer having 1 to 6 atoms in the main chain” for X is a straight chain connecting ring A and imidazole, and the atom number of the main chain is counted such that the number of atoms in the main chain will be minimum. The “main chain” consists of 1 to 6 atoms selected from a carbon atom and a hetero atom (e.g., O, S, N etc.), and may be saturated or unsaturated. In addition, S may be oxidized.

Examples of the “spacer having 1 to 6 atoms in the main chain” include straight chain C₁₋₆ alkylene, —X¹—NH—X²—, —X¹—O—X²— and —X¹—S—X²— [wherein X¹ and X² are the same or different and each is bond or straight chain C₁₋₅ alkylene, when X¹ and X² are both straight chain C₁₋₅ alkylene, then the total carbon number of straight chain C₁₋₅ alkylene for X¹ and straight chain C₁₋₅ alkylene for X² is 5 or less, and S is optionally oxidized] and the like.

Examples of the “straight chain C₁₋₆ alkylene” include —CH₂—, —CH₂CH₂—, —CH₂CH₂CH₂—, —CH₂CH₂CH₂CH₂—, —CH₂CH₂CH₂CH₂CH₂— and —CH₂CH₂CH₂CH₂CH₂CH₂—.

Examples of the “straight chain C₁₋₅ alkylene” for X¹ or X² include —CH₂—, —CH₂CH₂—, —CH₂CH₂CH₂—, —CH₂CH₂CH₂CH₂— and —CH₂CH₂CH₂CH₂CH₂—.

The “spacer having 1 to 6 atoms in the main chain” optionally has substituent(s) (preferably 1 to 3 substituents) at substitutable position(s) (optionally at the carbon atom and nitrogen atom constituting the main chain). Examples of the substituent include groups exemplified as the substituents which the aforementioned “hydrocarbon group” optionally has, and the like. When the number of the substituents is not less than 2, respective substituents may be the same or different.

X is preferably bond, C₁₋₆ alkylene optionally having substituent(s) or the like.

X is more preferably

(1) bond,
(2) C₁₋₆ alkylene optionally having substituent(s) selected from C₁₋₆ alkyl and C₆₋₁₀ aryl (e.g., phenyl) or the like.

Ring A is C₅₋₇ cycloalkane optionally having substituent(s).

Examples of the “C₅₋₇ cycloalkane” of the “C₅₋₇ cycloalkane optionally having substituent(s)” include cyclopropane, cyclobutane, cyclopentane, cyclohexane, bicyclo[1.1.1]pentane, bicyclo[2.1.1]hexane, bicyclo[2.2.1]heptane, bicyclo[3.1.1]heptane and the like.

The “C₅₋₇ cycloalkane” of the “C₅₋₇ cycloalkane optionally having substituent(s)” optionally has substituent(s) (e.g., 1 to 5, preferably 1 to 3 substituents) at substitutable position(s). When the number of the substituents is not less than 2, respective substituents may be the same or different, and may be substituted at the same carbon of ring A. In addition, two substituents may be bonded each other to form, with C₅₋₇ cycloalkane, an optionally substituted ring (a fused ring or spiro ring).

Examples of the fused ring or spiro ring include a fused ring or spiro ring consisting of C₅₋₇ cycloalkane and C₃₋₁₀ cycloalkane, C₃₋₁₀ cycloalkene, C₄₋₁₀ cycloalkadiene or a heterocycle. Examples of the “C₃₋₁₀ cycloalkane”, “C₃₋₁₀ cycloalkene”, “C₄₋₁₀ cycloalkadiene” and “heterocycle” include rings corresponding to the aforementioned C₃₋₁₀ cycloalkyl, C₃₋₁₀ cycloalkenyl, C₄₋₁₀ cycloalkadienyl and heterocyclic group.

Examples of the “substituent” of the “C₅₋₇ cycloalkane optionally having substituent(s)” include a halogen atom, a hydrocarbon group optionally having substituent(s), a heterocyclic group optionally having substituent(s), hydroxy optionally having a substituent, amino optionally having substituent(s), mercapto optionally having substituent(s), cyano, acyl and the like. Preferable example thereof include a halogen atom, a hydrocarbon group optionally having substituent(s), hydroxy optionally having a substituent, amino optionally having substituent(s) and the like. More preferable Example thereof include a halogen atom, a hydrocarbon group optionally having substituent(s), hydroxy optionally having a substituent and the like.

Ring A is preferably C₅₋₇ cycloalkane optionally having substituent(s) selected from a halogen atom, a hydrocarbon group optionally having substituent(s), hydroxy optionally having a substituent and amino optionally having substituent(s).

More preferably, ring A is

(a) C₅₋₇ cycloalkane having hydroxy optionally having a substituent, and optionally further having substituent(s) (e.g., a halogen atom, a hydrocarbon group optionally having substituent(s) etc.), or
(b) C₅₋₇ cycloalkane substituted by amino optionally having substituent(s) (e.g., a hydrocarbon group optionally having substituent(s), acyl etc.).

Further more preferably, ring A is

(a) C₅₋₇ cycloalkane having hydroxy optionally having a substituent, and optionally further having substituent(s) (e.g., C₁₋₃ alkyl optionally having substituent(s) etc.), or
(b) C₅₋₇ cycloalkane substituted by amino optionally having substituent(s) (e.g., C₁₋₆ alkoxy-carbonyl etc.).

Still more preferably, ring A is

(a) C₅₋₇ cycloalkane substituted by hydroxy optionally having a substituent, and optionally further substituted by C₁₋₃ alkyl optionally having substituent(s), or
(b) C₅₋₇ cycloalkane substituted by amino optionally having substituent(s) (e.g., C₁₋₆ alkoxy-carbonyl etc.).

Still more preferably, ring A is

(a) C₅₋₇ cycloalkane having hydroxy optionally having a substituent, and optionally further having substituent(s) (e.g., cyclopropylmethyl, methyl, methoxymethyl, ethoxymethyl etc.), or
(b) C₅₋₇ cycloalkane substituted by amino optionally having substituent(s) (e.g., methoxycarbonyl, ethoxycarbonyl etc.).

Preferable embodiments of ring A is the following [A] and [B] and the like.

[A]: C₅₋₇ cycloalkane optionally having 1 to 5 substituents selected from
(1) a halogen atom;
(2) C₁₋₆ alkyl optionally having 1 to 5 substituents selected from
(i) a halogen atom,
(ii) cyano,
(iii) C₃₋₆ cycloalkyl,
(iv) hydroxy,
(v) C₁₋₆ alkoxy optionally having 1 or 2 substituents selected from
   (a) a halogen atom,
   (b) hydroxy,
   (c) C₁₋₆ alkoxy optionally having 1 or 2 hydroxy,
   (d) C₃₋₆ cycloalkyl,
   (e) mono- or di-C₁₋₆ alkylamino,
   (f) C₁₋₆ alkyl-carbonylamino,
   (g) C₁₋₆ alkylthio,
   (h) C₁₋₆ alkylsulfonyl, and
   (i) a heterocyclic group (e.g., a 5- or 6-membered heterocyclic group such as thiazolyl, imidazolyl, pyrrolidinyl, oxazolidinyl, pyridyl, oxetanyl, tetrahydrothiopyranyl, tetrahydropyranyl and the like; benzimidazolyl; the heterocyclic group is optionally oxidized, e.g., 1,1-dioxidotetrahydrothiopyranyl) optionally having 1 or 2 substituents selected from C₁₋₆ alkyl and oxo,
(vi) C₃₋₆ cycloalkyloxy optionally condensed with a benzene ring (e.g., cyclobutyloxy, indanyloxy),
(vii) C₆₋₁₀ aryloxy (e.g., phenoxy),
(viii) 5- or 6-membered heterocyclyloxy (e.g., tetrahydropyranyloxy, piperidinyloxy, tetrahydrothiopyranyloxy; the heterocycle is optionally oxidized, e.g., 1,1-dioxidotetrahydrothiopyranyloxy) optionally having 1 or 2 substituents selected from C₁₋₆ alkyl and oxo,
(ix) C₁₋₆ alkyl-carbonyloxy,
(x) carboxy,
(xi) C₁₋₆ alkoxy-carbonyl,
(xii) carbamoyl optionally having 1 or 2 substituents selected from C₁₋₆ alkyl and 5- or 6-membered aromatic heterocyclyl-C₁₋₆ alkyl (e.g., furfuryl),
(xiii) C₁₋₆ alkylthio,
(xiv) C₁₋₆ alkylsulfonyl,
(xv) amino optionally having 1 or 2 substituents selected from C₁₋₆ alkyl, C₁₋₆ alkyl-carbonyl, C₁₋₆ alkoxy-carbonyl, 5- or 6-membered aromatic heterocyclyl-C₁₋₆ alkyl (e.g., furfuryl) and C₁₋₆ alkylsulfonyl-C₁₋₆ alkyl, and
(xvi) a 5- or 6-membered aromatic heterocyclic group (e.g., tetrazolyl);
   (3) hydroxy optionally having a substituent selected from
(i) C₇₋₁₃ aralkyl (e.g., benzyl),
(ii) C₁₋₆ alkyl optionally having 1 to 3 substituents selected from C₁₋₆ alkoxy and C₁₋₆ alkyl-carbonylamino,
(iii) C₂₋₆ alkenyl,
(iv) C₁₋₆ alkyl-carbonyl,
(v) C₆₋₁₀ aryl-carbonyl (e.g., benzoyl) optionally having 1 or 2 nitro, and
(vi) carbamoyl optionally having 1 or 2 substituents selected from C₁₋₆ alkyl, C₁₋₆ alkylsulfonyl-C₁₋₆ alkyl and 5- or 6-membered aromatic heterocyclyl-C₁₋₆ alkyl (e.g., furfuryl);
   (4) amino optionally having 1 or 2 substituents selected from C₁₋₆ alkyl, C₁₋₆ alkoxy-C₂₋₆ alkyl, C₃₋₆ cycloalkyl-C₁₋₆ alkyl, C₁₋₆ alkyl-carbonyl, C₃₋₆ cycloalkyl-carbonyl, C₁₋₆ alkoxy-carbonyl, C₁₋₆ alkoxy-C₁₋₆ alkoxy-carbonyl, mono- or di-C₁₋₆ alkyl-carbamoyl and C₃₋₆ cycloalkylsulfonyl;
   (5) 5- or 6-membered cyclic amino (e.g., morpholino, oxazolidinyl) optionally having 1 or 2 oxo;
   (6) C₁₋₃ alkylidene (e.g., methylene) optionally having a substituent selected from C₁₋₆ alkoxy-carbonyl and C₁₋₆ alkyl-carbamoyl;
   (7) oxo; and
   (8) azido;
   [B]: C₅₋₇ cycloalkane forming, together with a 5- or 6-membered non-aromatic heterocycle, a spiro ring (e.g., 1-oxa-3-azaspiro[4.5]decyl) optionally having 1 or 2 oxo.

More preferable embodiments of ring A is the following [A] and [B] and the like.

[A]: C₅₋₇ cycloalkane optionally having 1 to 5 substituents selected from
(1) a halogen atom;
(2) C₁₋₆ alkyl optionally having 1 to 5 substituents selected from
(i) a halogen atom,
(ii) cyano,
(iii) C₃₋₆ cycloalkyl,
(iv) hydroxy,
(v) C₁₋₆ alkoxy optionally having 1 or 2 substituents selected from
   (a) a halogen atom,
   (b) hydroxy,
   (c) C₁₋₆ alkoxy optionally having 1 or 2 hydroxy,
   (d) C₃₋₆ cycloalkyl,
   (e) mono- or di-C₁₋₆ alkylamino,
   (f) C₁₋₆ alkyl-carbonylamino,
   (g) C₁₋₆ alkylthio,
   (h) C₁₋₆ alkylsulfonyl, and
   (i) a heterocyclic group (e.g., a 5- or 6-membered heterocyclic group such as thiazolyl, imidazolyl, pyrrolidinyl, oxazolidinyl, pyridyl, oxetanyl, tetrahydrothiopyranyl, tetrahydropyranyl and the like; benzimidazolyl; the heterocyclic group is optionally oxidized, e.g., 1,1-dioxidotetrahydrothiopyranyl) optionally having 1 or 2 substituents selected from C₁₋₆ alkyl and oxo,
(vi) C₃₋₆ cycloalkyloxy optionally condensed with a benzene ring (e.g., cyclobutyloxy, indanyloxy),
(vii) C₆₋₁₀ aryloxy (e.g., phenoxy),
(viii) 5- or 6-membered heterocyclyloxy (e.g., tetrahydropyranyloxy, piperidinyloxy, tetrahydrothiopyranyloxy; the heterocycle is optionally oxidized, e.g., 1,1-dioxidotetrahydrothiopyranyloxy) optionally having 1 or 2 substituents selected from C₁₋₆ alkyl and oxo,
(ix) C₁₋₆ alkyl-carbonyloxy,
(x) carboxy,
(xi) C₁₋₆ alkoxy-carbonyl,
(xii) carbamoyl optionally having 1 or 2 substituents selected from C₁₋₆ alkyl and 5- or 6-membered aromatic heterocyclyl-C₁₋₆ alkyl (e.g., furfuryl),
(xiii) C₁₋₆ alkylthio,
(xiv) C₁₋₆ alkylsulfonyl,
(xv) amino optionally having 1 or 2 substituents selected from C₁₋₆ alkyl, C₁₋₆ alkyl-carbonyl, C₁₋₆ alkoxy-carbonyl, 5- or 6-membered aromatic heterocyclyl-C₁₋₆ alkyl (e.g., furfuryl) and C₁₋₆ alkylsulfonyl-C₁₋₆ alkyl, and
(xvi) a 5- or 6-membered aromatic heterocyclic group (e.g., tetrazolyl);
   (3) hydroxy optionally having a substituent selected from
(i) C₇₋₁₃ aralkyl (e.g., benzyl),
(ii) C₁₋₆ alkyl optionally having 1 to 3 substituents selected from C₁₋₆ alkoxy and C₁₋₆ alkyl-carbonylamino,
(iii) C₂₋₆ alkenyl,
(iv) C₁₋₆ alkyl-carbonyl,
(v) C₆₋₁₀ aryl-carbonyl (e.g., benzoyl) optionally having 1 or 2 nitro, and
(vi) carbamoyl optionally having 1 or 2 substituents selected from C₁₋₆ alkyl, C₁₋₆ alkylsulfonyl-C₁₋₆ alkyl and 5- or 6-membered aromatic heterocyclyl-C₁₋₆ alkyl (e.g., furfuryl);
   (4) amino optionally having 1 or 2 substituents selected from
(i) C₁₋₆ alkyl,
(ii) C₁₋₆ alkoxy-C₂₋₆ alkyl,
(iii) C₃₋₆ cycloalkyl-C₁₋₆ alkyl,
(iv) C₁₋₆ alkyl-carbonyl,
(v) C₃₋₆ cycloalkyl-carbonyl,
(vi) C₁₋₆ alkoxy-carbonyl optionally having 1 to 3 substituents selected from a halogen atom, C₁₋₆ alkoxy and C₃₋₆ cycloalkyl,
(vii) C₃₋₆ cycloalkoxy-carbonyl,
(viii) C₁₋₆ alkoxy-C₁₋₆ alkoxy-carbonyl,
(ix) mono- or di-C₁₋₆ alkyl-carbamoyl,
(x) C₃₋₆ cycloalkylsulfonyl,
(xi) C₁₋₆ alkylsulfonyl, and
(xii) mono- or di-C₁₋₆ alkylsulfamoyl;
   (5) 5- or 6-membered cyclic amino (e.g., morpholino, oxazolidinyl) optionally having 1 or 2 oxo;
   (6) C₁₋₃ alkylidene (e.g., methylene) optionally having a substituent selected from C₁₋₆ alkoxy-carbonyl and C₁₋₆ alkyl-carbamoyl;
   (7) oxo; and
   (8) azido;
   [B]: C₅₋₇ cycloalkane forming, together with a 5- or 6-membered non-aromatic heterocycle, a spiro ring (e.g., 1-oxa-3-azaspiro[4.5]decyl) optionally having 1 or 2 oxo.

Ring B is piperazine optionally further having substituent(s) besides R¹.

Examples of the “substituent” which ring B optionally further has include groups exemplified as the “substituent” for R¹ optionally has, and the like. Specific examples of the “substituent” include optionally substituted C₁₋₆ alkyl, for example, C₁₋₆ alkyl optionally having a 5- or 6-membered non-aromatic heterocyclic group (e.g., dioxolyl) optionally having 1 to 3 substituents selected from C₁₋₆ alkyl and oxo, and the like.

Ring B is preferably a ring represented by the formula:

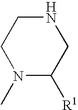

wherein R¹ is as defined above. The piperazine ring optionally has 1 to 3 C₁₋₆ alkyl at the ring-constituting carbon atom.

Preferable examples of compound (I) include the following compounds.

### [Compound A]

Compound (I) wherein

R¹ is a hydrocarbon group optionally having substituent(s);

R² is C₆₋₁₄ aryl optionally having substituent(s) or C₃₋₁₀ cycloalkyl optionally having substituent(s);

R³ is a hydrogen atom, a halogen atom, C₁₋₃ alkyl or C₁₋₃ alkoxy;

X is bond or C₁₋₆ alkylene optionally having substituent(s); and

ring A is C₅₋₇ cycloalkane optionally having substituent(s) selected from a halogen atom, a hydrocarbon group optionally having substituent(s), hydroxy optionally having a substituent and amino optionally having substituent(s).

### [Compound B]

A compound represented by the formula:

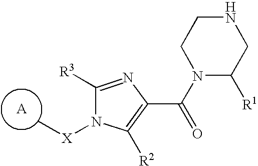

wherein

R¹ is

(a) C₁₋₆ alkyl substituted by hydroxy optionally having a substituent (e.g., a fused aromatic heterocyclic group such as benzofuranyl (e.g., 5-benzofuranyl, 6-benzofuranyl), benzothienyl (e.g., 5-benzothienyl, 6-benzothienyl), benzoxazolyl (e.g., 5-benzoxazolyl, 6-benzoxazolyl), benzisoxazolyl (e.g., 5-benzisoxazolyl, 6-benzisoxazolyl), benzothiazolyl (e.g., 5-benzothiazolyl, 6-benzothiazolyl), benzimidazolyl (e.g., benzimidazol-5-yl), benzotriazolyl (e.g., 1H-1,2,3-benzotriazol-5-yl), indolyl (e.g., indol-5-yl, indol-6-yl), indazolyl (e.g., 1H-indazol-5-yl, 1H-indazol-6-yl), pyrrolopyrazinyl (e.g., 1H-pyrrolo[2,3-b]pyrazin-2-yl, 1H-pyrrolo[2,3-b]pyrazin-6-yl), imidazopyridyl (e.g., 1H-imidazo[4,5-b]pyridin-5-yl, 1H-imidazo[4,5-c]pyridin-5-yl, 2H-imidazo[1,2-a]pyridin-5-yl), imidazopyrazinyl (e.g., 1H-imidazo[4,5-b]pyrazin-5-yl), pyrazolopyridyl (e.g., 1H-pyrazolo[4,3-c]pyridin-5-yl), pyrazolothienyl (e.g., 2H-pyrazolo[3,4-b]thiophen-2-yl) and the like),
(b) C₁₋₆ alkyl substituted by phenylamino optionally having substituent(s) (e.g., a halogen atom, C₁₋₆ alkyl optionally having substituent(s), C₁₋₆ alkoxy optionally having substituent(s)), or
(c) C₇₋₁₃ aralkyl optionally having substituent(s) (e.g., a halogen atom, C₁₋₆ alkyl optionally having substituent(s), C₁₋₆ alkoxy optionally having substituent(s), a monocyclic aromatic heterocyclic group optionally having substituent(s));

R² is optionally halogenated C₆₋₁₀ aryl (e.g., phenyl), or C₃₋₆ cycloalkyl (e.g., cyclopropyl, cyclohexyl);

R³ is a hydrogen atom;

X is

(1) bond, or
(2) C₁₋₆ alkylene optionally having substituent(s) selected from C₁₋₆ alkyl and C₆₋₁₀ aryl (e.g., phenyl); and

ring A is

(a) C₅₋₇ cycloalkane having hydroxy optionally having a substituent, and optionally further having substituent(s) (e.g., C₁₋₃ alkyl optionally having substituent(s) etc.), or
(b) C₅₋₇ cycloalkane substituted by amino optionally having substituent(s) (e.g., C₁₋₆ alkoxy-carbonyl etc.).

### [Compound B′]

A compound represented by the formula:

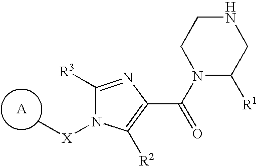

wherein

R¹ is

(a) C₁₋₆ alkyl substituted by hydroxy optionally having a substituent (e.g., a fused aromatic heterocyclic group such as benzofuranyl (e.g., 5-benzofuranyl, 6-benzofuranyl), benzothienyl (e.g., 5-benzothienyl, 6-benzothienyl), benzoxazolyl (e.g., 5-benzoxazolyl, 6-benzoxazolyl), benzisoxazolyl (e.g., 5-benzisoxazolyl, 6-benzisoxazolyl), benzothiazolyl (e.g., 5-benzothiazolyl, 6-benzothiazolyl), benzimidazolyl (e.g., benzimidazol-5-yl), benzotriazolyl (e.g., 1H-1,2,3-benzotriazol-5-yl), indolyl (e.g., indol-5-yl, indol-6-yl), indazolyl (e.g., 1H-indazol-5-yl, 1H-indazol-6-yl), pyrrolopyrazinyl (e.g., 1H-pyrrolo[2,3-b]pyrazin-2-yl, 1H-pyrrolo[2,3-b]pyrazin-6-yl), imidazopyridyl (e.g., 1H-imidazo[4,5-b]pyridin-5-yl, 1H-imidazo[4,5-c]pyridin-5-yl, 2H-imidazo[1,2-a]pyridin-5-yl), imidazopyrazinyl (e.g., 1H-imidazo[4,5-b]pyrazin-5-yl), pyrazolopyridyl (e.g., 1H-pyrazolo[4,3-c]pyridin-5-yl), pyrazolothienyl (e.g., 2H-pyrazolo[3,4-b]thiophen-2-yl) and the like),
(b) C₁₋₆ alkyl substituted by phenylamino optionally having substituent(s) (e.g., a halogen atom, C₁₋₆ alkyl optionally having substituent(s), C₁₋₆ alkoxy optionally having substituent(s)), or
(c) C₇₋₁₃ aralkyl optionally having substituent(s) (e.g., a halogen atom, C₁₋₆ alkyl optionally having substituent(s), C₁₋₆ alkoxy optionally having substituent(s), a monocyclic aromatic heterocyclic group optionally having substituent(s));

R² is optionally halogenated C₆₋₁₀ aryl (e.g., phenyl);

R³ is a hydrogen atom, a halogen atom, C₁₋₃ alkyl (e.g., methyl, ethyl, propyl, isopropyl) or C₁₋₃ alkoxy (e.g., methoxy, ethoxy, propoxy, isopropoxy);

X is

(1) bond, or
(2) C₁₋₆ alkylene optionally having substituent(s) (e.g., C₁₋₆ alkyl, C₆₋₁₀ aryl (e.g., phenyl), etc.); and

ring A is

(a) C₅₋₇ cycloalkane substituted by hydroxy optionally having a substituent, and optionally further substituted by C₁₋₃ alkyl optionally having substituent(s), or
(b) C₅₋₇ cycloalkane substituted by amino optionally having substituent(s) (e.g., C₁₋₆ alkoxy-carbonyl etc.).

### [Compound B′-1]

Compound B′ wherein R¹ is C₁₋₆ alkyl substituted by hydroxy optionally having a substituent (e.g., a fused aromatic heterocyclic group such as benzofuranyl (e.g., 5-benzofuranyl, 6-benzofuranyl), benzothienyl (e.g., 5-benzothienyl, 6-benzothienyl), benzoxazolyl (e.g., 5-benzoxazolyl, 6-benzoxazolyl), benzisoxazolyl (e.g., 5-benzisoxazolyl, 6-benzisoxazolyl), benzothiazolyl (e.g., 5-benzothiazolyl, 6-benzothiazolyl), benzimidazolyl (e.g., benzimidazol-5-yl), benzotriazolyl (e.g., 1H-1,2,3-benzotriazol-5-yl), indolyl (e.g., indol-5-yl, indol-6-yl), indazolyl (e.g., 1H-indazol-5-yl, 1H-indazol-6-yl), pyrrolopyrazinyl (e.g., 1H-pyrrolo[2,3-b]pyrazin-2-yl, 1H-pyrrolo[2,3-b]pyrazin-6-yl), imidazopyridyl (e.g., 1H-imidazo[4,5-b]pyridin-5-yl, 1H-imidazo[4,5-c]pyridin-5-yl, 2H-imidazo[1,2-a]pyridin-5-yl), imidazopyrazinyl (e.g., 1H-imidazo[4,5-b]pyrazin-5-yl), pyrazolopyridyl (e.g., 1H-pyrazolo[4,3-c]pyridin-5-yl), pyrazolothienyl (e.g., 2H-pyrazolo[3,4-b]thiophen-2-yl) and the like).

### [Compound B′-2]

Compound B′ wherein R¹ is C₁₋₆ alkyl substituted by phenylamino optionally having substituent(s) (e.g., a halogen atom, C₁₋₆ alkyl optionally having substituent(s), C₁₋₆ alkoxy optionally having substituent(s)).

### [Compound B′-3]

Compound B′ wherein R¹ is C₇₋₁₃ aralkyl optionally having substituent(s) (e.g., a halogen atom, C₁₋₆ alkyl optionally having substituent(s), C₁₋₆ alkoxy optionally having substituent(s), a monocyclic aromatic heterocyclic group optionally having substituent(s)).

### [Compound B′-4]

Compound B′ wherein ring A is C₅₋₇ cycloalkane substituted by hydroxy optionally having a substituent, and optionally further substituted by C₁₋₃ alkyl optionally having substituent(s).

### [Compound B′-4]

Compound B′ wherein ring A is C₅₋₇ cycloalkane substituted by amino optionally having substituent(s) (e.g., C₁₋₆ alkoxy-carbonyl etc.).

### [Compound C]

A compound represented by the formula:

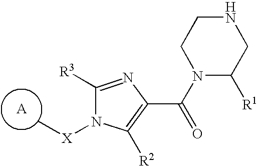

wherein

R¹ is

(1) C₇₋₁₃ aralkyl (e.g., benzyl, phenethyl, phenylpropyl) optionally having 1 to 3 substituents selected from
(i) a halogen atom,
(ii) C₁₋₆ alkyl optionally having 1 to 5 substituents selected from a halogen atom and hydroxy,
(iii) cyano,
(iv) hydroxy,
(v) optionally halogenated C₁₋₆ alkoxy (e.g., trifluoromethoxy), and
(vi) a 5- or 6-membered non-aromatic heterocyclic group (e.g., morpholinyl);
   (2) C₃₋₁₀ cycloalkyl-C₁₋₆ alkyl (e.g., cyclopropylmethyl, cyclohexylmethyl) optionally having one hydroxy;
   (3) C₁₋₆ alkyl optionally having 1 to 5 substituents selected from
(i) a halogen atom,
(ii) hydroxy optionally having a substituent selected from
   (a) C₆₋₁₀ aryl (e.g., phenyl) optionally having 1 to 3 substituents selected from
      A) a halogen atom,
      B) cyano,
      C) C₁₋₆ alkyl optionally having 1 or 2 substituents selected from carboxy, hydroxy, C₁₋₆ alkoxy-carbonyl and mono- or di-C₁₋₆ alkylamino,
      D) optionally halogenated C₁₋₆ alkoxy (e.g., methoxy, trifluoromethoxy, ethoxy, isopropoxy),
      E) C₁₋₄ alkylenedioxy,
      F) carboxy,
      G) C₁₋₆ alkyl-carbonyl,
      H) C₁₋₆ alkoxy-carbonyl,
      I) 5- or 6-membered non-aromatic heterocyclylcarbonyl (e.g., azetidinylcarbonyl),
      J) carbamoyl,
      K) optionally halogenated mono- or di-C₁₋₆ alkyl-carbamoyl,
      L) C₃₋₆ cycloalkyl-carbamoyl (e.g., cyclopropylcarbamoyl),
      M) mono- or di-C₁₋₆ alkylamino,
      O) optionally halogenated C₁₋₆ alkylsulfonyl (e.g., methylsulfonyl, trifluoromethylsulfonyl), and
      P) a 5- or 6-membered heterocyclic group (e.g., imidazolyl, pyrazolyl, triazolyl, oxadiazolyl, pyrrolidinyl, piperazinyl) optionally having 1 or 2 substituents selected from C₁₋₆ alkyl, C₁₋₆ alkyl-carbonyl and oxo,
   (b) C₆₋₁₀ aryl condensed with C₃₋₁₀ cycloalkane (e.g., tetrahydronaphthyl) optionally having 1 or 2 oxo,
   (c) a 5- or 6-membered aromatic heterocyclic group (e.g., pyrazolyl, triazolyl, thienyl, thiazolyl, isoxazolyl, pyridyl, pyrimidinyl; the 5- or 6-membered aromatic heterocyclic group is optionally oxidized) optionally having 1 to 3 substituents selected from a halogen atom, cyano, optionally halogenated C₁₋₆ alkyl, C₁₋₆ alkoxy-carbonyl-C₁₋₆ alkyl, mono- or di-C₁₋₆ alkylamino-C₁₋₆ alkyl (e.g., dimethylaminomethyl), C₆₋₁₀ aryl (e.g., phenyl), C₁₋₆ alkoxy-carbonyl and carboxy,
   (d) a 9- or 10-membered fused heterocyclic group (e.g., benzothiazolyl, dihydrobenzoxazolyl, benzisoxazolyl, dihydrobenzofuranyl, tetrahydroquinolyl, tetrahydroisoquinolyl, chromenyl, thienopyridyl) optionally having 1 to 3 substituents selected from C₁₋₆ alkyl, C₁₋₆ alkoxy and oxo,
   (e) C₇₋₁₃ aralkyl (e.g., benzyl),
   (f) C₃₋₁₀ cycloalkyl-C₁₋₆ alkyl (e.g., cyclopropylmethyl), and
   (g) 5- or 6-membered non-aromatic heterocyclylcarbonyl (e.g., pyrrolidinylcarbonyl),
(iii) C₆₋₁₀ arylthio (e.g., phenylthio),
(iv) amino optionally having 1 or 2 substituents selected from
   (a) C₁₋₆ alkyl,
   (b) C₆₋₁₀ aryl (e.g., phenyl),
   (c) C₃₋₆ cycloalkyl-carbonyl,
   (d) C₆₋₁₀ aryl-carbonyl optionally having 1 to 3 substituents selected from a halogen atom and C₁₋₆ alkoxy,
   (e) C₁₋₆ alkoxy-carbonyl-C₁₋₆ alkyl-carbonyl, and
   (f) carbamoyl-C₁₋₆ alkyl-carbonyl,
(v) a 5- or 6-membered heterocyclic group (e.g., piperidinyl, pyrrolyl, imidazolyl, pyrazolyl, triazolyl, thiazolyl, oxadiazolyl, pyridyl) optionally having 1 to 3 substituents selected from
   (a) C₁₋₆ alkyl optionally having 1 to 5 substituents selected from a halogen atom, hydroxy and C₁₋₆ alkyl-carbonyloxy,
   (b) C₃₋₆ cycloalkyl,
   (c) C₆₋₁₀ aryl (e.g., phenyl),
   (d) C₁₋₆ alkyl-carbonyl, and
   (e) C₁₋₆ alkoxy-carbonyl, and
(vi) a 9- or 10-membered fused heterocyclic group (e.g., indolyl, indazolyl, dihydroindazolyl, tetrahydroindazolyl, benzotriazolyl, benzimidazolyl, dihydrobenzimidazolyl, dihydrobenzoxazolyl, dihydrobenzoxazinyl) optionally having 1 to 3 substituents selected from cyano, C₁₋₆ alkyl, C₃₋₆ cycloalkyl, C₁₋₆ alkoxy-carbonyl and oxo; or
   (4) C₃₋₁₀ cycloalkyl optionally condensed with a benzene ring (e.g., indanyl);

R² is optionally halogenated C₆₋₁₀ aryl (e.g., phenyl), or C₃₋₆ cycloalkyl (e.g., cyclopropyl, cyclohexyl);

R³ is a hydrogen atom, a halogen atom, C₁₋₃ alkyl or C₁₋₃ alkoxy;

X is

(1) bond, or
(2) C₁₋₆ alkylene optionally having substituent(s) selected from C₁₋₆ alkyl and C₆₋₁₀ aryl (e.g., phenyl); and

ring A is

[A] C₅₋₇ cycloalkane optionally having 1 to 5 substituents selected from the following (1) to (8), or [B] C₅₋₇ cycloalkane forming, together with a 5- or 6-membered non-aromatic heterocycle, a spiro ring (e.g., 1-oxa-3-azaspiro[4.5]decyl) optionally having 1 or 2 oxo:
(1) a halogen atom;
(2) C₁₋₆ alkyl optionally having 1 to 5 substituents selected from
(i) a halogen atom,
(ii) cyano,
(iii) C₃₋₆ cycloalkyl,
(iv) hydroxy,
(v) C₁₋₆ alkoxy optionally having 1 or 2 substituents selected from
   (a) a halogen atom,
   (b) hydroxy,
   (c) C₁₋₆ alkoxy optionally having 1 or 2 hydroxy,
   (d) C₃₋₆ cycloalkyl,
   (e) mono- or di-C₁₋₆ alkylamino,
   (f) C₁₋₆ alkyl-carbonylamino,
   (g) C₁₋₆ alkylthio,
   (h) C₁₋₆ alkylsulfonyl, and
   (i) a heterocyclic group (e.g., a 5- or 6-membered heterocyclic group such as thiazolyl, imidazolyl, pyrrolidinyl, oxazolidinyl, pyridyl, oxetanyl, tetrahydrothiopyranyl, tetrahydropyranyl and the like; benzimidazolyl; the heterocyclic group is optionally oxidized, e.g., 1,1-dioxidotetrahydrothiopyranyl) optionally having 1 or 2 substituents selected from C₁₋₆ alkyl and oxo,
(vi) C₃₋₆ cycloalkyloxy optionally condensed with a benzene ring (e.g., cyclobutyloxy, indanyloxy),
(vii) C₆₋₁₀ aryloxy (e.g., phenoxy),
(viii) 5- or 6-membered heterocyclyloxy (e.g., tetrahydropyranyloxy, piperidinyloxy, tetrahydrothiopyranyloxy; the heterocycle is optionally oxidized, e.g., 1,1-dioxidotetrahydrothiopyranyloxy) optionally having 1 or 2 substituents selected from C₁₋₆ alkyl and oxo,
(ix) C₁₋₆ alkyl-carbonyloxy,
(x) carboxy,
(xi) C₁₋₆ alkoxy-carbonyl,
(xii) carbamoyl optionally having 1 or 2 substituents selected from C₁₋₆ alkyl and 5- or 6-membered aromatic heterocyclyl-C₁₋₆ alkyl (e.g., furfuryl),
(xiii) C₁₋₆ alkylthio,
(xiv) C₁₋₆ alkylsulfonyl,
(xv) amino optionally having 1 or 2 substituents selected from C₁₋₆ alkyl, C₁₋₆ alkyl-carbonyl, C₁₋₆ alkoxy-carbonyl, 5- or 6-membered aromatic heterocyclyl-C₁₋₆ alkyl (e.g., furfuryl) and C₁₋₆ alkylsulfonyl-C₁₋₆ alkyl, and
(xvi) a 5- or 6-membered aromatic heterocyclic group (e.g., tetrazolyl);
   (3) hydroxy optionally having a substituent selected from
(i) C₇₋₁₃ aralkyl (e.g., benzyl),
(ii) C₁₋₆ alkyl optionally having 1 to 3 substituents selected from C₁₋₆ alkoxy and C₁₋₆ alkyl-carbonylamino,
(iii) C₂₋₆ alkenyl,
(iv) C₁₋₆ alkyl-carbonyl,
(v) C₆₋₁₀ aryl-carbonyl (e.g., benzoyl) optionally having 1 or 2 nitro, and
(vi) carbamoyl optionally having 1 or 2 substituents selected from C₁₋₆ alkyl, C₁₋₆ alkylsulfonyl-C₁₋₆ alkyl and 5- or 6-membered aromatic heterocyclyl-C₁₋₆ alkyl (e.g., furfuryl);
   (4) amino optionally having 1 or 2 substituents selected from C₁₋₆ alkyl, C₁₋₆ alkoxy-C₂₋₆ alkyl, C₃₋₆ cycloalkyl-C₁₋₆ alkyl, C₁₋₆ alkyl-carbonyl, C₃₋₆ cycloalkyl-carbonyl, C₁₋₆ alkoxy-carbonyl, C₁₋₆ alkoxy-C₁₋₆ alkoxy-carbonyl, mono- or di-C₁₋₆ alkyl-carbamoyl and C₃₋₆ cycloalkylsulfonyl;
   (5) 5- or 6-membered cyclic amino (e.g., morpholino, oxazolidinyl) optionally having 1 or 2 oxo;
   (6) C₁₋₃ alkylidene (e.g., methylene) optionally having a substituent selected from C₁₋₆ alkoxy-carbonyl and C₁₋₆ alkyl-carbamoyl;
   (7) oxo; and
   (8) azido.

### [Compound D]

Compound (I) wherein

R¹ is

(1) C₇₋₁₃ aralkyl (e.g., benzyl, phenethyl, phenylpropyl, naphthylmethyl, biphenylylmethyl) optionally having 1 to 3 substituents selected from
(i) a halogen atom,
(ii) C₁₋₆ alkyl optionally having 1 to 5 substituents selected from a halogen atom and hydroxy,
(iii) cyano,
(iv) hydroxy,
(v) optionally halogenated C₁₋₆ alkoxy (e.g., methoxy, trifluoromethoxy),
(vi) C₆₋₁₀ aryloxy (e.g., phenoxy),
(vii) a 5- or 6-membered non-aromatic heterocyclic group (e.g., morpholinyl), and
(viii) a 5- or 6-membered aromatic heterocyclic group (e.g., pyridyl, pyrazolyl) optionally having 1 to 3 substituents selected from C₁₋₆ alkyl and C₁₋₆ alkoxy;
   (2) C₃₋₁₀ cycloalkyl-C₁₋₆ alkyl (e.g., cyclopropylmethyl, cyclohexylmethyl) optionally having one hydroxy;
   (3) C₁₋₆ alkyl optionally having 1 to 5 substituents selected from
(i) a halogen atom,
(ii) hydroxy optionally having a substituent selected from
   (a) C₆₋₁₀ aryl (e.g., phenyl) optionally having 1 to 3 substituents selected from
      A) a halogen atom,
      B) cyano,
      C) C₁₋₆ alkyl optionally having 1 or 2 substituents selected from carboxy, hydroxy, C₁₋₆ alkoxy-carbonyl and mono- or di-C₁₋₆ alkylamino,
      D) optionally halogenated C₁₋₆ alkoxy (e.g., methoxy, trifluoromethoxy, ethoxy, isopropoxy, difluoromethoxy),
      E) C₁₋₄ alkylenedioxy,
      F) carboxy,
      G) C₁₋₆ alkyl-carbonyl,
      H) C₁₋₆ alkoxy-carbonyl,
      I) 5- or 6-membered non-aromatic heterocyclylcarbonyl (e.g., azetidinylcarbonyl),
      J) carbamoyl,
      K) optionally halogenated mono- or di-C₁₋₆ alkyl-carbamoyl,
      L) C₃₋₆ cycloalkyl-carbamoyl (e.g., cyclopropylcarbamoyl),
      M) mono- or di-C₁₋₆ alkylamino,
      O) optionally halogenated C₁₋₆ alkylsulfonyl (e.g., methylsulfonyl, trifluoromethylsulfonyl),
      P) a 5- or 6-membered heterocyclic group (e.g., imidazolyl, pyrazolyl, triazolyl, oxadiazolyl, pyrrolidinyl, piperazinyl, morpholinyl) optionally having 1 or 2 substituents selected from C₁₋₆ alkyl, C₁₋₆ alkyl-carbonyl and oxo, and
      Q) a 9- or 10-membered fused heterocyclic group (e.g., dihydroimidazoimidazolyl) optionally having 1 to 3 substituents selected from C₁₋₆ alkyl and oxo,
   (b) C₆₋₁₀ aryl condensed with C₃₋₁₀ cycloalkane (e.g., tetrahydronaphthyl) optionally having 1 or 2 oxo,
   (c) a 5- or 6-membered aromatic heterocyclic group (e.g., pyrazolyl, triazolyl, thienyl, thiazolyl, isoxazolyl, pyridyl, pyrimidinyl; the 5- or 6-membered aromatic heterocyclic group is optionally oxidized) optionally having 1 to 3 substituents selected from a halogen atom, cyano, optionally halogenated C₁₋₆ alkyl, C₁₋₆ alkoxy-carbonyl-C₁₋₆ alkyl, mono- or di-C₁₋₆ alkylamino-C₁₋₆ alkyl (e.g., dimethylaminomethyl), C₆₋₁₀ aryl (e.g., phenyl), C₁₋₆ alkoxy-carbonyl and carboxy,
   (d) a 9- or 10-membered fused heterocyclic group (e.g., benzothiazolyl, benzimidazolyl, benzothienyl, dihydrobenzoxazolyl, benzisoxazolyl, benzofuranyl, dihydrobenzofuranyl, tetrahydroquinolyl, tetrahydroisoquinolyl, chromenyl, thienopyridyl) optionally having 1 to 3 substituents selected from C₁₋₆ alkyl, C₁₋₆ alkoxy, C₁₋₆ alkoxy-carbonyl, a halogen atom and oxo,
   (e) C₇₋₁₃ aralkyl (e.g., benzyl),
   (f) C₃₋₁₀ cycloalkyl-C₁₋₆ alkyl (e.g., cyclopropylmethyl),
   (g) 5- or 6-membered non-aromatic heterocyclylcarbonyl (e.g., pyrrolidinylcarbonyl), and
   (h) C₆₋₁₀ aryl-carbamoyl (e.g., phenylcarbamoyl),
(iii) C₆₋₁₀ arylthio (e.g., phenylthio),
(iv) C₆₋₁₀ arylsulfinyl (e.g., phenylsulfinyl),
(v) optionally halogenated C₆₋₁₀ arylsulfonyl (e.g., phenylsulfonyl, fluorophenylsulfonyl),
(vi) amino optionally having 1 or 2 substituents selected from
   (a) C₁₋₆ alkyl,
   (b) C₆₋₁₀ aryl (e.g., phenyl) optionally having 1 to 3 substituents selected from
      A) a halogen atom,
      B) optionally halogenated C₁₋₆ alkyl (e.g., isopropyl, trifluoromethyl),
      C) optionally halogenated C₁₋₆ alkoxy (e.g., methoxy, trifluoromethoxy, difluoromethoxy),
      D) cyano,
      E) nitro,
      F) carboxy,
      G) C₁₋₆ alkyl-carbonyl,
      H) C₁₋₆ alkoxy-carbonyl,
      I) C₁₋₄ alkylenedioxy, and
      J) a 5- or 6-membered heterocyclic group (e.g., pyrazolyl, piperidinyl, dihydropyridyl) optionally having 1 or 2 oxo,
   (c) C₃₋₆ cycloalkyl optionally condensed with a benzene ring (e.g., cyclopropyl, cyclohexyl, indanyl),
   (d) C₇₋₁₃ aralkyl (e.g., benzyl),
   (e) C₁₋₆ alkyl-carbonyl,
   (f) C₃₋₆ cycloalkyl-carbonyl,
   (g) C₆₋₁₀ aryl-carbonyl optionally having 1 to 3 substituents selected from a halogen atom and C₁₋₆ alkoxy,
   (h) C₁₋₆ alkoxy-carbonyl-C₁₋₆ alkyl-carbonyl,
   (i) carbamoyl-C₁₋₆ alkyl-carbonyl,
   (j) a 5- or 6-membered heterocyclic group (e.g., pyridyl), and
   (k) a 9- or 10-membered fused heterocyclic group (e.g., benzoxazolyl, benzothiazolyl, dihydrobenzofuranyl, indazolyl, dihydrofuropyridyl) optionally having 1 to 3 substituents selected from C₁₋₆ alkyl and oxo,
(vii) a 5- or 6-membered heterocyclic group (e.g., piperidinyl, pyrrolyl, imidazolyl, pyrazolyl, triazolyl, thiazolyl, oxadiazolyl, pyridyl) optionally having 1 to 3 substituents selected from
   (a) C₁₋₆ alkyl optionally having 1 to 5 substituents selected from a halogen atom, hydroxy and C₁₋₆ alkyl-carbonyloxy,
   (b) C₃₋₆ cycloalkyl,
   (c) C₆₋₁₀ aryl (e.g., phenyl),
   (d) C₁₋₆ alkyl-carbonyl, and
   (e) C₁₋₆ alkoxy-carbonyl,
(viii) a 9- or 10-membered fused heterocyclic group (e.g., indolyl, dihydroisoindolyl, indazolyl, dihydroindazolyl, tetrahydroindazolyl, benzotriazolyl, benzimidazolyl, dihydrobenzimidazolyl, dihydrobenzoxazolyl, dihydrobenzoxazinyl, tetrahydroquinolyl, tetrahydroisoquinolyl) optionally having 1 to 3 substituents selected from cyano, C₁₋₆ alkyl, C₃₋₆ cycloalkyl, C₁₋₆ alkoxy-carbonyl and oxo,
(ix) carbamoyl optionally having 1 or 2 substituents selected from C₁₋₆ alkyl and C₆₋₁₀ aryl,
(x) 5- or 6-membered heterocyclylthio (e.g., thiazolylthio, thiadiazolylthio, triazolylthio) optionally having C₁₋₆ alkyl optionally having 1 to 3 substituents selected from hydroxy and C₁₋₆ alkyl-carbonyloxy, and
(xi) a 9- or 10-membered fused heterocyclylthio (e.g., benzothiazolylthio, benzimidazolylthio, thiazolopyridylthio); or
   (4) C₃₋₁₀ cycloalkyl optionally condensed with a benzene ring (e.g., indanyl);

R² is optionally halogenated C₆₋₁₀ aryl (e.g., phenyl), or C₃₋₆ cycloalkyl (e.g., cyclopropyl, cyclohexyl);

R³ is a hydrogen atom, a halogen atom, C₁₋₃ alkyl or C₁₋₃ alkoxy;

X is

(1) bond, or
(2) C₁₋₆ alkylene optionally having substituent(s) selected from C₁₋₆ alkyl and C₆₋₁₀ aryl (e.g., phenyl);

ring A is

[A] C₅₋₇ cycloalkane optionally having 1 to 5 substituents selected from the following (1) to (8), or [B] C₅₋₇ cycloalkane forming, together with a 5- or 6-membered non-aromatic heterocycle, a spiro ring (e.g., 1-oxa-3-azaspiro[4.5]decyl) optionally having 1 or 2 oxo:
(1) a halogen atom;
(2) C₁₋₆ alkyl optionally having 1 to 5 substituents selected from
(i) a halogen atom,
(ii) cyano,
(iii) C₃₋₆ cycloalkyl,
(iv) hydroxy,
(v) C₁₋₆ alkoxy optionally having 1 or 2 substituents selected from
   (a) a halogen atom,
   (b) hydroxy,
   (c) C₁₋₆ alkoxy optionally having 1 or 2 hydroxy,
   (d) C₃₋₆ cycloalkyl,
   (e) mono- or di-C₁₋₆ alkylamino,
   (f) C₁₋₆ alkyl-carbonylamino,
   (g) C₁₋₆ alkylthio,
   (h) C₁₋₆ alkylsulfonyl, and
   (i) a heterocyclic group (e.g., a 5- or 6-membered heterocyclic group such as thiazolyl, imidazolyl, pyrrolidinyl, oxazolidinyl, pyridyl, oxetanyl, tetrahydrothiopyranyl, tetrahydropyranyl and the like; benzimidazolyl; the heterocyclic group is optionally oxidized, e.g., 1,1-dioxidotetrahydrothiopyranyl) optionally having 1 or 2 substituents selected from C₁₋₆ alkyl and oxo,
(vi) C₃₋₆ cycloalkyloxy optionally condensed with a benzene ring (e.g., cyclobutyloxy, indanyloxy),
(vii) C₆₋₁₀ aryloxy (e.g., phenoxy),
(viii) 5- or 6-membered heterocyclyloxy (e.g., tetrahydropyranyloxy, piperidinyloxy, tetrahydrothiopyranyloxy; the heterocycle is optionally oxidized, e.g., 1,1-dioxidotetrahydrothiopyranyloxy) optionally having 1 or 2 substituents selected from C₁₋₆ alkyl and oxo,
(ix) C₁₋₆ alkyl-carbonyloxy,
(x) carboxy,
(xi) C₁₋₆ alkoxy-carbonyl,
(xii) carbamoyl optionally having 1 or 2 substituents selected from C₁₋₆ alkyl and 5- or 6-membered aromatic heterocyclyl-C₁₋₆ alkyl (e.g., furfuryl),
(xiii) C₁₋₆ alkylthio,
(xiv) C₁₋₆ alkylsulfonyl,
(xv) amino optionally having 1 or 2 substituents selected from C₁₋₆ alkyl, C₁₋₆ alkyl-carbonyl, C₁₋₆ alkoxy-carbonyl, 5- or 6-membered aromatic heterocyclyl-C₁₋₆ alkyl (e.g., furfuryl) and C₁₋₆ alkylsulfonyl-C₁₋₆ alkyl, and
(xvi) a 5- or 6-membered aromatic heterocyclic group (e.g., tetrazolyl);
   (3) hydroxy optionally having a substituent selected from
(i) C₇₋₁₃ aralkyl (e.g., benzyl),
(ii) C₁₋₆ alkyl optionally having 1 to 3 substituents selected from C₁₋₆ alkoxy and C₁₋₆ alkyl-carbonylamino,
(iii) C₂₋₆ alkenyl,
(iv) C₁₋₆ alkyl-carbonyl,
(v) C₆₋₁₀ aryl-carbonyl (e.g., benzoyl) optionally having 1 or 2 nitro, and
(vi) carbamoyl optionally having 1 or 2 substituents selected from C₁₋₆ alkyl, C₁₋₆ alkylsulfonyl-C₁₋₆ alkyl and 5- or 6-membered aromatic heterocyclyl-C₁₋₆ alkyl (e.g., furfuryl);
   (4) amino optionally having 1 or 2 substituents selected from C₁₋₆ alkyl, C₁₋₆ alkoxy-C₂₋₆ alkyl, C₃₋₆ cycloalkyl-C₁₋₆ alkyl, C₁₋₆ alkyl-carbonyl, C₃₋₆ cycloalkyl-carbonyl, C₁₋₆ alkoxy-carbonyl, C₁₋₆ alkoxy-C₁₋₆ alkoxy-carbonyl, mono- or di-C₁₋₆ alkyl-carbamoyl and C₃₋₆ cycloalkylsulfonyl;
   (5) 5- or 6-membered cyclic amino (e.g., morpholino, oxazolidinyl) optionally having 1 or 2 oxo;
   (6) C₁₋₃ alkylidene (e.g., methylene) optionally having a substituent selected from C₁₋₆ alkoxy-carbonyl and C₁₋₆ alkyl-carbamoyl;
   (7) oxo; and
   (8) azido; and

ring B is piperazine optionally further having, besides R¹, C₁₋₆ alkyl optionally having a 5- or 6-membered non-aromatic heterocyclic group (e.g., dioxolyl) optionally having 1 to 3 substituents selected from C₁₋₆ alkyl and oxo.

### [Compound E]

Compound (I) wherein

R¹ is

(1) C₇₋₁₃ aralkyl (e.g., benzyl, phenethyl, phenylpropyl, naphthylmethyl, biphenylylmethyl) optionally having 1 to 3 substituents selected from
(i) a halogen atom,
(ii) C₁₋₆ alkyl optionally having 1 to 5 substituents selected from a halogen atom and hydroxy,
(iii) cyano,
(iv) hydroxy,
(v) optionally halogenated C₁₋₆ alkoxy (e.g., methoxy, trifluoromethoxy),
(vi) C₆₋₁₀ aryloxy (e.g., phenoxy),
(vii) a 5- or 6-membered non-aromatic heterocyclic group (e.g., morpholinyl), and
(viii) a 5- or 6-membered aromatic heterocyclic group (e.g., pyridyl, pyrazolyl) optionally having 1 to 3 substituents selected from C₁₋₆ alkyl and C₁₋₆ alkoxy;
   (2) C₃₋₁₀ cycloalkyl-C₁₋₆ alkyl (e.g., cyclopropylmethyl, cyclohexylmethyl) optionally having one hydroxy;
   (3) C₁₋₆ alkyl optionally having 1 to 5 substituents selected from
(i) a halogen atom,
(ii) hydroxy optionally having a substituent selected from
   (a) C₆₋₁₀ aryl (e.g., phenyl, naphthyl) optionally having 1 to 3 substituents selected from
      A) a halogen atom,
      B) cyano,
      C) C₁₋₆ alkyl optionally having 1 to 3 substituents selected from a halogen atom, carboxy, hydroxy, C₁₋₆ alkoxy-carbonyl and mono- or di-C₁₋₆ alkylamino,
      D) C₁₋₆ alkoxy optionally having 1 to 3 substituents selected from a halogen atom and C₁₋₆ alkoxy,
      E) C₁₋₄ alkylenedioxy,
      F) carboxy,
      G) C₁₋₆ alkyl-carbonyl,
      H) C₁₋₆ alkoxy-carbonyl,
      I) 5- or 6-membered non-aromatic heterocyclylcarbonyl (e.g., azetidinylcarbonyl),
      J) carbamoyl,
      K) optionally halogenated mono- or di-C₁₋₆ alkyl-carbamoyl,
      L) C₃₋₆ cycloalkyl-carbamoyl (e.g., cyclopropylcarbamoyl),
      M) mono- or di-C₁₋₆ alkylamino,
      O) optionally halogenated C₁₋₆ alkylsulfonyl (e.g., methylsulfonyl, trifluoromethylsulfonyl),
      P) a 5- or 6-membered heterocyclic group (e.g., imidazolyl, pyrazolyl, triazolyl, oxadiazolyl, pyrrolidinyl, piperazinyl, morpholinyl) optionally having 1 or 2 substituents selected from C₁₋₆ alkyl, C₁₋₆ alkyl-carbonyl and oxo, and
      Q) a 9- or 10-membered fused heterocyclic group (e.g., dihydroimidazoimidazolyl) optionally having 1 to 3 substituents selected from C₁₋₆ alkyl and oxo,
   (b) C₆₋₁₀ aryl condensed with C₃₋₁₀ cycloalkane (e.g., tetrahydronaphthyl) optionally having 1 or 2 oxo,
   (c) a 5- or 6-membered aromatic heterocyclic group (e.g., pyrazolyl, triazolyl, thienyl, thiazolyl, isoxazolyl, pyridyl, pyrimidinyl; the 5- or 6-membered aromatic heterocyclic group is optionally oxidized) optionally having 1 to 3 substituents selected from a halogen atom, cyano, optionally halogenated C₁₋₆ alkyl, C₁₋₆ alkoxy-carbonyl-C₁₋₆ alkyl, mono- or di-C₁₋₆ alkylamino-C₁₋₆ alkyl (e.g., dimethylaminomethyl), C₆₋₁₀ aryl (e.g., phenyl), C₁₋₆ alkoxy-carbonyl and carboxy,
   (d) a 9- or 10-membered fused heterocyclic group (e.g., benzothiazolyl, benzimidazolyl, benzothienyl, benzoxazolyl, indolyl, dihydrobenzoxazolyl, benzisoxazolyl, benzofuranyl, dihydrobenzofuranyl, tetrahydroquinolyl, tetrahydroisoquinolyl, chromenyl, thienopyridyl, dihydrobenzoxazinyl) optionally having 1 to 3 substituents selected from
      A) C₁₋₆ alkyl optionally having 1 to 3 substituents selected from C₁₋₆ alkoxy and C₁₋₆ alkoxy-carbonyl,
      B) C₁₋₆ alkoxy,
      C) C₁₋₆ alkoxy-carbonyl,
      D) C₃₋₁₀ cycloalkyl,
      E) a halogen atom, and
      F) oxo,
   (e) C₇₋₁₃ aralkyl (e.g., benzyl),
   (f) C₃₋₁₀ cycloalkyl-C₁₋₆ alkyl (e.g., cyclopropylmethyl),
   (g) 5- or 6-membered non-aromatic heterocyclylcarbonyl (e.g., pyrrolidinylcarbonyl),
   (h) C₆₋₁₀ aryl-carbamoyl (e.g., phenylcarbamoyl), and
   (i) C₃₋₆ cycloalkyl optionally condensed with a benzene ring (e.g., indanyl),
(iii) C₆₋₁₀ arylthio (e.g., phenylthio),
(iv) C₆₋₁₀ arylsulfinyl (e.g., phenylsulfinyl),
(v) optionally halogenated C₆₋₁₀ arylsulfonyl (e.g., phenylsulfonyl, fluorophenylsulfonyl),
(vi) amino optionally having 1 or 2 substituents selected from
   (a) C₁₋₆ alkyl,
   (b) C₆₋₁₀ aryl (e.g., phenyl) optionally having 1 to 3 substituents selected from
      A) a halogen atom,
      B) optionally halogenated C₁₋₆ alkyl (e.g., methyl, isopropyl, trifluoromethyl),
      C) optionally halogenated C₁₋₆ alkoxy (e.g., methoxy, trifluoromethoxy, difluoromethoxy),
      D) cyano,
      E) nitro,
      F) carboxy,
      G) C₁₋₆ alkyl-carbonyl,
      H) C₁₋₆ alkoxy-carbonyl,
      I) C₁₋₄ alkylenedioxy, and
      J) a 5- or 6-membered heterocyclic group (e.g., pyrazolyl, piperidinyl, dihydropyridyl) optionally having 1 or 2 oxo,
   (c) C₃₋₆ cycloalkyl optionally condensed with a benzene ring (e.g., cyclopropyl, cyclohexyl, indanyl),
   (d) C₇₋₁₃ aralkyl (e.g., benzyl),
   (e) C₁₋₆ alkyl-carbonyl,
   (f) C₃₋₆ cycloalkyl-carbonyl,
   (g) C₆₋₁₀ aryl-carbonyl optionally having 1 to 3 substituents selected from a halogen atom and C₁₋₆ alkoxy,
   (h) C₁₋₆ alkoxy-carbonyl-C₁₋₆ alkyl-carbonyl,
   (i) carbamoyl-C₁₋₆ alkyl-carbonyl,
   (j) a 5- or 6-membered heterocyclic group (e.g., pyridyl), and
   (k) a 9- or 10-membered fused heterocyclic group (e.g., benzoxazolyl, benzothiazolyl, dihydrobenzofuranyl, indazolyl, dihydrofuropyridyl) optionally having 1 to 3 substituents selected from C₁₋₆ alkyl and oxo,
(vii) a 5- or 6-membered heterocyclic group (e.g., piperidinyl, pyrrolyl, imidazolyl, pyrazolyl, triazolyl, thiazolyl, oxadiazolyl, pyridyl, tetrazolyl) optionally having 1 to 3 substituents selected from
   (a) C₁₋₆ alkyl optionally having 1 to 5 substituents selected from a halogen atom, hydroxy and C₁₋₆ alkyl-carbonyloxy,
   (b) C₃₋₆ cycloalkyl,
   (c) C₆₋₁₀ aryl (e.g., phenyl),
   (d) C₁₋₆ alkyl-carbonyl, and
   (e) C₁₋₆ alkoxy-carbonyl,
(viii) a 9- or 10-membered fused heterocyclic group (e.g., indolyl, dihydroisoindolyl, indazolyl, dihydroindazolyl, tetrahydroindazolyl, benzotriazolyl, benzimidazolyl, dihydrobenzimidazolyl, dihydrobenzoxazolyl, dihydrobenzoxazinyl, tetrahydroquinolyl, tetrahydroisoquinolyl) optionally having 1 to 3 substituents selected from cyano, C₁₋₆ alkyl, C₃₋₆ cycloalkyl, C₁₋₆ alkoxy-carbonyl and oxo,
(ix) carbamoyl optionally having 1 or 2 substituents selected from C₁₋₆ alkyl and C₆₋₁₀ aryl,
(x) 5- or 6-membered heterocyclylthio (e.g., thiazolylthio, thiadiazolylthio, triazolylthio) optionally having C₁₋₆ alkyl optionally having 1 to 3 substituents selected from hydroxy and C₁₋₆ alkyl-carbonyloxy, and
(xi) 9- or 10-membered fused heterocyclylthio (e.g., benzothiazolylthio, benzimidazolylthio, thiazolopyridylthio); or
   (4) C₃₋₁₀ cycloalkyl optionally condensed with a benzene ring (e.g., indanyl);

R² is optionally halogenated C₆₋₁₀ aryl (e.g., phenyl), or C₃₋₆ cycloalkyl (e.g., cyclopropyl, cyclohexyl);

R³ is a hydrogen atom, a halogen atom, C₁₋₃ alkyl or C₁₋₃ alkoxy;

X is

(1) bond, or
(2) C₁₋₆ alkylene optionally having substituent(s) selected from C₁₋₆ alkyl and C₆₋₁₀ aryl (e.g., phenyl);

ring A is

[A] C₅₋₇ cycloalkane optionally having 1 to 5 substituents selected from the following (1) to (8), or [B] C₅₋₇ cycloalkane forming, together with a 5- or 6-membered non-aromatic heterocycle, a spiro ring (e.g., 1-oxa-3-azaspiro[4.5]decyl) optionally having 1 or 2 oxo:
(1) a halogen atom;
(2) C₁₋₆ alkyl optionally having 1 to 5 substituents selected from
(i) a halogen atom,
(ii) cyano,
(iii) C₃₋₆ cycloalkyl,
(iv) hydroxy,
(v) C₁₋₆ alkoxy optionally having 1 or 2 substituents selected from
   (a) a halogen atom,
   (b) hydroxy,
   (c) C₁₋₆ alkoxy optionally having 1 or 2 hydroxy,
   (d) C₃₋₆ cycloalkyl,
   (e) mono- or di-C₁₋₆ alkylamino,
   (f) C₁₋₆ alkyl-carbonylamino,
   (g) C₁₋₆ alkylthio,
   (h) C₁₋₆ alkylsulfonyl, and
   (i) a heterocyclic group (e.g., a 5- or 6-membered heterocyclic group such as thiazolyl, imidazolyl, pyrrolidinyl, oxazolidinyl, pyridyl, oxetanyl, tetrahydrothiopyranyl, tetrahydropyranyl and the like; benzimidazolyl; the heterocyclic group is optionally oxidized, e.g., 1,1-dioxidotetrahydrothiopyranyl) optionally having 1 or 2 substituents selected from C₁₋₆ alkyl and oxo,
(vi) C₃₋₆ cycloalkyloxy optionally condensed with a benzene ring (e.g., cyclobutyloxy, indanyloxy),
(vii) C₆₋₁₀ aryloxy (e.g., phenoxy),
(viii) 5- or 6-membered heterocyclyloxy (e.g., tetrahydropyranyloxy, piperidinyloxy, tetrahydrothiopyranyloxy; the heterocycle is optionally oxidized, e.g., 1,1-dioxidotetrahydrothiopyranyloxy) optionally having 1 or 2 substituents selected from C₁₋₆ alkyl and oxo,
(ix) C₁₋₆ alkyl-carbonyloxy,
(x) carboxy,
(xi) C₁₋₆ alkoxy-carbonyl,
(xii) carbamoyl optionally having 1 or 2 substituents selected from C₁₋₆ alkyl and 5- or 6-membered aromatic heterocyclyl-C₁₋₆ alkyl (e.g., furfuryl),
(xiii) C₁₋₆ alkylthio,
(xiv) C₁₋₆ alkylsulfonyl,
(xv) amino optionally having 1 or 2 substituents selected from C₁₋₆ alkyl, C₁₋₆ alkyl-carbonyl, C₁₋₆ alkoxy-carbonyl, 5- or 6-membered aromatic heterocyclyl-C₁₋₁₆ alkyl (e.g., furfuryl) and C₁₋₆ alkylsulfonyl-C₁₋₆ alkyl, and
(xvi) a 5- or 6-membered aromatic heterocyclic group (e.g., tetrazolyl);
   (3) hydroxy optionally having a substituent selected from
(i) C₇₋₁₃ aralkyl (e.g., benzyl),
(ii) C₁₋₆ alkyl optionally having 1 to 3 substituents selected from C₁₋₆ alkoxy and C₁₋₆ alkyl-carbonylamino,
(iii) C₂₋₆ alkenyl,
(iv) C₁₋₆ alkyl-carbonyl,
(v) C₆₋₁₀ aryl-carbonyl (e.g., benzoyl) optionally having 1 or 2 nitro, and
(vi) carbamoyl optionally having 1 or 2 substituents selected from C₁₋₆ alkyl, C₁₋₆ alkylsulfonyl-C₁₋₆ alkyl and 5- or 6-membered aromatic heterocyclyl-C₁₋₆ alkyl (e.g., furfuryl);
   (4) amino optionally having 1 or 2 substituents selected from
(i) C₁₋₆ alkyl,
(ii) C₁₋₆ alkoxy-C₂₋₆ alkyl,
(iii) C₃₋₆ cycloalkyl-C₁₋₆ alkyl,
(iv) C₁₋₆ alkyl-carbonyl,
(v) C₃₋₆ cycloalkyl-carbonyl,
(vi) C₁₋₆ alkoxy-carbonyl optionally having 1 to 3 substituents selected from a halogen atom, C₁₋₆ alkoxy and C₃₋₆ cycloalkyl,
(vii) C₃₋₆ cycloalkoxy-carbonyl,
(viii) C₁₋₆ alkoxy-C₁₋₆ alkoxy-carbonyl,
(ix) mono- or di-C₁₋₆ alkyl-carbamoyl,
(X) C₃₋₆ cycloalkylsulfonyl,
(xi) C₁₋₆ alkylsulfonyl, and
(xii) mono- or di-C₁₋₆ alkylsulfamoyl;
   (5) 5- or 6-membered cyclic amino (e.g., morpholino, oxazolidinyl) optionally having 1 or 2 oxo;
   (6) C₁₋₃ alkylidene (e.g., methylene) optionally having a substituent selected from C₁₋₆ alkoxy-carbonyl and C₁₋₆ alkyl-carbamoyl;
   (7) oxo; and
   (8) azido; and

ring B is piperazine optionally further having, besides R¹, C₁₋₆ alkyl optionally having a 5- or 6-membered non-aromatic heterocyclic group (e.g., dioxolyl) optionally having 1 to 3 substituents selected from C₁₋₆ alkyl and oxo.

Specific examples of compound (I) include
methyl [(1S,2S)-2-(4-{[(2R)-2-(3,5-difluorobenzyl)piperazin-1-yl]carbonyl}-5-phenyl-1H-imidazol-1-yl)cyclohexyl]carbamate,
(1S,2R)-2-{4-[((2R)-2-{2-[(2-fluoro-4-methoxyphenyl)amino]ethyl}piperazin-1-yl)carbonyl]-5-phenyl-1H-imidazol-1-yl}-1-(methoxymethyl)cyclohexanol,
(1S,2R)-2-(4-{[(2R)-2-benzylpiperazin-1-yl]carbonyl}-5-phenyl-1H-imidazol-1-yl)-1-methylcyclohexanol dihydrochloride,
(1S,2R)-1-(methoxymethyl)-2-{4-[((2R)-2-{2-[(4-methoxy-2-methylphenyl)amino]ethyl}piperazin-1-yl)carbonyl]-5-phenyl-1H-imidazol-1-yl}cyclohexanol,
(1S,2R)-1-(methoxymethyl)-2-{4-[((2R)-2-{2-[(5-methoxy-2-methylphenyl)amino]ethyl}piperazin-1-yl)carbonyl]-5-phenyl-1H-imidazol-1-yl}cyclohexanol,
(1S,2R)-1-(methoxymethyl)-2-{4-[((2R)-2-{2-[(2-methoxy-5-methylphenyl)amino]ethyl}piperazin-1-yl)carbonyl]-5-phenyl-1H-imidazol-1-yl}cyclohexanol,
(1S,2R)-2-[4-{[(2R)-2-benzylpiperazin-1-yl]carbonyl}-5-(3-fluorophenyl)-1H-imidazol-1-yl]-1-(methoxymethyl)cyclohexanol,
(1S,2R)-1-(methoxymethyl)-2-{4-[((2R)-2-{2-[(2-methylphenyl)amino]ethyl}piperazin-1-yl)carbonyl]-5-phenyl-1H-imidazol-1-yl}cyclohexanol,
(1S,2R)-2-{4-[((2R)-2-{2-[(3-fluoro-2-methoxyphenyl)amino]ethyl}piperazin-1-yl)carbonyl]-5-phenyl-1H-imidazol-1-yl}-1-(methoxymethyl)cyclohexanol,
(1S,2R)-2-{4-[((2R)-2-{2-[(2-ethyl-1,3-benzoxazol-5-yl)amino]ethyl}piperazin-1-yl)carbonyl]-5-phenyl-1H-imidazol-1-yl}-1-(methoxymethyl)cyclohexanol,
(1S,2R)-2-{4-[((2R)-2-{2-[(2-fluorophenyl)amino]ethyl}piperazin-1-yl)carbonyl]-5-phenyl-1H-imidazol-1-yl}-1-(methoxymethyl)cyclohexanol,
(1S,2R)-1-(methoxymethyl)-2-[4-({(2R)-2-[2-(2-methoxy-4-methylphenoxy)ethyl]piperazin-1-yl}carbonyl)-5-phenyl-1H-imidazol-1-yl]cyclohexanol dihydrochloride,
(1S,2R)-1-(methoxymethyl)-2-{4-[((2R)-2-{2-[(4-methoxyphenyl)amino]ethyl}piperazin-1-yl)carbonyl]-5-phenyl-1H-imidazol-1-yl}cyclohexanol,
(1S,2R)-1-(methoxymethyl)-2-{4-[((2R)-2-{2-[(2-methyl-1,3-benzothiazol-5-yl)oxy]ethyl}piperazin-1-yl)carbonyl]-5-phenyl-1H-imidazol-1-yl}cyclohexanol hydrochloride,
(1S,2R)-2-{4-[((2R)-2-{2-[(3-fluoro-4-methoxyphenyl)amino]ethyl}piperazin-1-yl)carbonyl]-5-phenyl-1H-imidazol-1-yl}-1-(methoxymethyl)cyclohexanol trihydrochloride,
(1S,2R)-2-{4-[((2R)-2-{2-[(2-fluoro-3-methoxyphenyl)amino]ethyl}piperazin-1-yl)carbonyl]-5-phenyl-1H-imidazol-1-yl}-1-(methoxymethyl)cyclohexanol,
(1S,2R)-1-(methoxymethyl)-2-(4-{[(2R)-2-(2-morpholinobenzyl)piperazin-1-yl]carbonyl}-5-phenyl-1H-imidazol-1-yl)cyclohexanol,
(1S,2R)-2-{4-[((2R)-2-{2-[(4-fluoro-2-methoxyphenyl)amino]ethyl}piperazin-1-yl)carbonyl]-5-phenyl-1H-imidazol-1-yl}-1-(methoxymethyl)cyclohexanol,
1-[(4-{[(2R)-2-(2-anilinoethyl)piperazin-1-yl]carbonyl}-5-phenyl-1H-imidazol-1-yl)methyl]cyclohexanol,
(1S,2R)-1-(methoxymethyl)-2-{4-[((2R)-2-{2-[(2-methoxyphenyl)amino]ethyl}piperazin-1-yl)carbonyl]-5-phenyl-1H-imidazol-1-yl}cyclohexanol,
(1S,2R)-1-(methoxymethyl)-2-[5-phenyl-4-({(2R)-2-[(5-phenyl-1,3,4-oxadiazol-2-yl)methyl]piperazin-1-yl}carbonyl)-1H-imidazol-1-yl]cyclohexanol hydrochloride,
(1S,2R)-1-(methoxymethyl)-2-{4-[((2R)-2-{2-[(3-methoxyphenyl)amino]ethyl}piperazin-1-yl)carbonyl]-5-phenyl-1H-imidazol-1-yl}cyclohexanol,
(1S,2R)-1-(methoxymethyl)-2-(5-phenyl-4-{([(2R)-2-(2-{[4-(1H-pyrazol-1-yl)phenyl]amino}ethyl)piperazin-1-yl]carbonyl}-1H-imidazol-1-yl)cyclohexanol, and
methyl [(1S,2S)-2-(4-{[(2R)-2-(2-anilinoethyl)piperazin-1-yl]carbonyl}-5-phenyl-1H-imidazol-1-yl)cyclohexyl]carbamate,
   and a salt thereof, and the like.

Another specific examples of compound (I) include
(1S,2R)-1-(methoxymethyl)-2-{(4-[((2R)-2-{2-[(2-methyl-1,3-benzothiazol-5-yl)oxy]ethyl}piperazin-1-yl)carbonyl]-5-phenyl-1H-imidazol-1-yl}cyclohexanol hydrochloride,
methyl [(1S,2S)-2-(4-{[(2R)-2-(3,5-difluorobenzyl)piperazin-1-yl]carbonyl}-5-phenyl-1H-imidazol-1-yl)cyclohexyl]carbamate,
(1S,2R)-1-(methoxymethyl)-2-[5-phenyl-4-({(2R)-2-[(5-phenyl-1,3,4-oxadiazol-2-yl)methyl]piperazin-1-yl}carbonyl)-1H-imidazol-1-yl]cyclohexanol hydrochloride,
(1S,2R)-1-(methoxymethyl)-2-[4-({(2R)-2-[2-(2-methoxy-4-methylphenoxy)ethyl]piperazin-1-yl}carbonyl)-5-phenyl-1H-imidazol-1-yl]cyclohexanol dihydrochloride,
ethyl [(1S,2S)-2-(4-{[(2R)-2-(3,5-difluorobenzyl)piperazin-1-yl]carbonyl}-5-phenyl-1H-imidazol-1-yl)cyclohexyl]carbamate,
ethyl [(1S,2S)-2-(4-{[(2R)-2-(3,5-difluorobenzyl)piperazin-1-yl]carbonyl}-5-phenyl-1H-imidazol-1-yl)cyclohexyl]carbamate malonate,
(1S,2R)-2-(4-{[(2R)-2-benzylpiperazin-1-yl]carbonyl}-5-phenyl-1H-imidazol-1-yl)-1-(methoxymethyl)cyclohexanol dihydrochloride,
(1S,2R)-2-(4-{[(2R)-2-benzylpiperazin-1-yl]carbonyl}-5-phenyl-1H-imidazol-1-yl)-1-(methoxymethyl)cyclohexanol fumarate,
(1S,2R)-2-[4-{[(2R)-2-benzylpiperazin-1-yl]carbonyl}-5-(3-fluorophenyl)-1H-imidazol-1-yl]-1-(methoxymethyl)cyclohexanol,
methyl [(1S,2S)-2-(4-{[(2R)-2-(2-anilinoethyl)piperazin-1-yl]carbonyl}-5-phenyl-1H-imidazol-1-yl)cyclohexyl]carbamate,
(1S,2R)-1-(methoxymethyl)-2-(4-{[(2R)-2-(2-morpholinobenzyl)piperazin-1-yl]carbonyl}-5-phenyl-1H-imidazol-1-yl)cyclohexanol,
(1S,2R)-2-(4-{[(2R)-2-benzylpiperazin-1-yl]carbonyl}-5-phenyl-1H-imidazol-1-yl)-1-methylcyclohexanol dihydrochloride,
(1S,2R)-1-(methoxymethyl)-2-{4-[((2R)-2-{2-[(3-methoxyphenyl)amino]ethyl}piperazin-1-yl)carbonyl]-5-phenyl-1H-imidazol-1-yl}cyclohexanol,
(1S,2R)-1-(methoxymethyl)-2-(5-phenyl-4-{[(2R)-2-(2-{[4-(1H-pyrazol-1-yl)phenyl]amino}ethyl)piperazin-1-yl]carbonyl}-1H-imidazol-1-yl)cyclohexanol,
(1S,2R)-1-(methoxymethyl)-2-{4-[((2R)-2-{2-[(5-methoxy-2-methylphenyl)amino]ethyl}piperazin-1-yl)carbonyl]-5-phenyl-1H-imidazol-1-yl}cyclohexanol,
(1S,2R)-2-{4-[((2R)-2-{2-[(2-ethyl-1,3-benzoxazol-5-yl)amino]ethyl}piperazin-1-yl)carbonyl]-5-phenyl-1H-imidazol-1-yl}-1-(methoxymethyl)cyclohexanol,
1-[(4-{[(2R)-2-(2-anilinoethyl)piperazin-1-yl]carbonyl}-5-phenyl-1H-imidazol-1-yl)methyl]cyclohexanol,
(1S,2R)-1-(methoxymethyl)-2-{4-[((2R)-2-{2-[(2-methyl-1,3-benzothiazol-5-yl)amino]ethyl}piperazin-1-yl)carbonyl]-5-phenyl-1H-imidazol-1-yl}cyclohexanol,
(1S,2R)-1-(methoxymethyl)-2-{4-[((2R)-2-{2-[(2-methyl-1,3-benzoxazol-5-yl)oxy]ethyl}piperazin-1-yl)carbonyl]-5-phenyl-1H-imidazol-1-yl}cyclohexanol,
(1S,2R)-2-{4-[((2R)-2-{2-[(3-fluoro-2-methoxyphenyl)amino]ethyl}piperazin-1-yl)carbonyl]-5-phenyl-1H-imidazol-1-yl}-1-(methoxymethyl)cyclohexanol,
ethyl ((1R,2R)-2-{4-[((2R)-2-{2-[(2-fluoro-3-methoxyphenyl)amino]ethyl}piperazin-1-yl)carbonyl]-5-phenyl-1H-imidazol-1-yl}cyclohexyl)carbamate,
(1S,2R)-2-{4-[((2R)-2-{2-[(2-fluoro-3-methoxyphenyl)amino]ethyl}piperazin-1-yl)carbonyl]-5-phenyl-1H-imidazol-1-yl}-1-(methoxymethyl)cyclohexanol,
propyl [(1S,2S)-2-(4-{[(2R)-2-(3,5-difluorobenzyl)piperazin-1-yl]carbonyl}-5-phenyl-1H-imidazol-1-yl)cyclohexyl]carbamate,
4-{[(2R)-1-({1-[(1R,2R)-2-(cyclopropylmethyl)-2-hydroxycyclohexyl]-5-phenyl-1H-imidazol-4-yl}carbonyl)piperazin-2-yl]methyl}benzonitrile,
isopropyl [(1S,2S)-2-(4-{[(2R)-2-(3,5-difluorobenzyl)piperazin-1-yl]carbonyl}-5-phenyl-1H-imidazol-1-yl)cyclohexyl]carbamate,
isopropyl [(1S,2S)-2-(4-{[(2R)-2-{2-[(2-fluoro-3-methoxyphenyl)amino]ethyl}piperazin-1-yl]carbonyl}-5-phenyl-1H-imidazol-1-yl)cyclohexyl]carbamate, and
(1S,2R)-1-(methoxymethyl)-2-(4-{[(2R)-2-{2-[(2-methyl-1,3-benzoxazol-6-yl)oxy]ethyl}piperazin-1-yl]carbonyl}-5-phenyl-1H-imidazol-1-yl)cyclohexanol,
   and a salt thereof, and the like.

Examples of the salt of compound (I) include metal salts, ammonium salts, salts with organic bases, salts with inorganic acids, salts with organic acids, salts with basic or acidic amino acids, and the like.

Preferable examples of the metal salt include alkali metal salts such as sodium salt, potassium salt and the like; alkaline earth metal salts such as calcium salt, magnesium salt, barium salt and the like; aluminum salt and the like.

Preferable examples of the salt with organic base include a salt with trimethylamine, triethylamine, pyridine, picoline, 2,6-lutidine, ethanolamine, diethanolamine, triethanolamine, cyclohexylamine, dicyclohexylamine, N,N-dibenzylethylenediamine or the like.

Preferable examples of the salt with inorganic acid include a salt with hydrochloric acid, hydrobromic acid, nitric acid, sulfuric acid, phosphoric acid or the like.

Preferable examples of the salt with organic acid include a salt with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid or the like.

Preferable examples of the salt with basic amino acid include a salt with arginine, lysine, ornithine or the like.

Preferable examples of the salt with acidic amino acid include a salt with aspartic acid, glutamic acid or the like.

Of these, a pharmaceutically acceptable salt is preferable. When the compound has an acidic functional group, examples thereof include inorganic salts such as alkali metal salts (e.g., sodium salt, potassium salt, etc.), alkaline earth metal salts (e.g., calcium salt, magnesium salt, barium salt, etc.) and the like, ammonium salts, and the like. When the compound has a basic functional group, examples thereof include salts with inorganic acids such as hydrochloric acid, hydrobromic acid, nitric acid, sulfuric acid, phosphoric acid and the like, and salts with organic acids such as acetic acid, phthalic acid, fumaric acid, oxalic acid, tartaric acid, maleic acid, citric acid, succinic acid, methanesulfonic acid, p-toluenesulfonic acid and the like.

The production methods of compound (I) are shown in the following.

Compound (I) is obtained by, for example, a method shown in the following reaction scheme or a method analogous thereto, or the like.

Each of compounds (II)-(VIII) shown in the reaction scheme may form a salt. Examples of the salt include salts similar to the salts of compound (I).

The compound obtained in each step can also be used for the next reaction directly as the reaction mixture or as a crude product. In addition, it can also be isolated from the reaction mixture according to a conventional method, and can be isolated and purified by a known method such as phase transfer, concentration, solvent extraction, fractional distillation, pH conversion, crystallization, recrystallization, chromatography and the like.

The schematic drawings of the reaction scheme are shown in the following.

R is C₁₋₄ alkyl, Y is a hydrogen atom or an alkali metal atom, PG is an N-protecting group (e.g., benzyl, tert-butoxycarbonyl, benzyloxycarbonyl etc.), and the other symbols are as defined above.

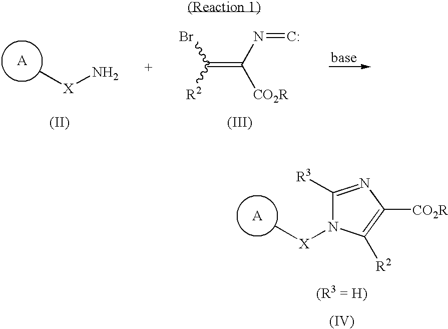

This method is used for the production of compound (IV) wherein R³ is a hydrogen atom.

Compound (II) may be commercially available, or can be produced according to a method known per se, for example, the method described in Tetrahedron: Asymmetry, 1997, vol. 8, pages 3153-3159, or the like, or a method analogous thereto.

The production of compound (III), and the production of compound (IV) by the reaction of compound (II) with compound (III) are performed, for example, according to the method described in Journal of Organic Chemistry, 1994, vol. 59, pages 7635-7642, or the like, or a method analogous thereto.

Compound (IV) wherein R³ is a halogen atom, C₁₋₆ alkyl or C₁₋₆ alkoxy can be produced according to a method known per se, for example, the method described in Journal of Organic Chemistry, 2004, vol. 69, pages 8829-8835, or the like, or a method analogous thereto.

Compound (IV) can be modified by further carrying out one or more of known acylation reaction, alkylation reaction, amination reaction, oxidation-reduction reaction, cyclization reaction, carbon chain extension reaction, substituent exchange reaction and the like, as desired.

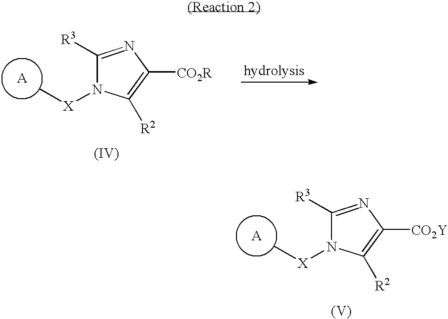

Compound (V) can be produced by subjecting compound (IV) to known hydrolysis, for example, alkali-hydrolysis or acid-hydrolysis.

The reaction is advantageously carried out under alkali conditions. Preferable examples of the alkali to be used for this step include alkali metal hydroxides such as lithium hydroxide, sodium hydroxide, potassium hydroxide and the like. The amount of the alkali to be used is about 1 mol to large excess, preferably 1 to 5 mol, per 1 mol of compound (IV).

The reaction is advantageously carried out in an inert solvent. While the solvent is not particularly limited as long as the reaction proceeds, preferable examples of the solvent include alcohols such as methanol, ethanol, propanol and the like; hydrocarbons such as benzene, toluene, cyclohexane, hexane and the like; halogenated hydrocarbons such as dichloromethane, chloroform, carbon tetrachloride, 1,2-dichloroethane and the like; ethers such as diethyl ether, tetrahydrofuran, dioxane and the like, a mixed solvent thereof, and the like.

While the reaction time varies depending on the reagent or solvent to be used, it is generally 30 min to 24 hr, preferably 30 min to 8 hr.

The reaction temperature is generally 0 to 150° C., preferably 20 to 80° C.

After the reaction, compound (V) (wherein Y is a hydrogen atom) is obtained as a free form by neutralizing the reaction mixture with a mineral acid (e.g., hydrochloric acid, sulfuric acid etc.), an organic acid (e.g., acetic acid etc.) or an ion exchange resin. Alternatively, compound (V) (wherein Y is an alkali metal atom such as lithium, sodium, potassium and the like) is obtained as an alkali metal salt of the carboxylic acid by directly concentrating the reaction mixture.

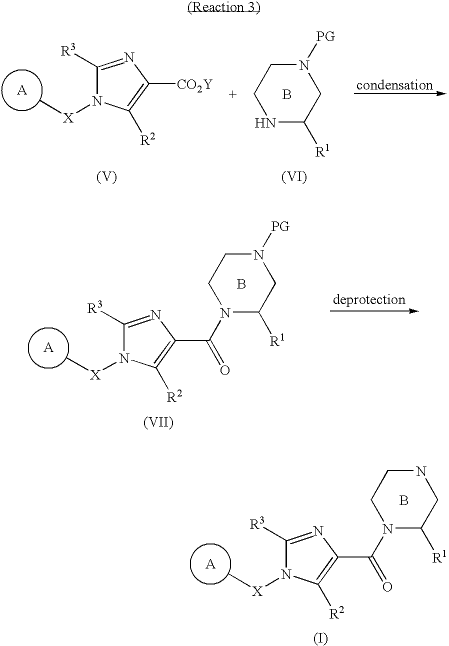

Compound (VII) can be produced by a condensation reaction of compound (V) with compound (VI).

Compound (VI) may be commercially available, or can be produced according to a method known per se, for example, the method described in WO 2003/000181 or the like, or a method analogous thereto.

When Y is a hydrogen atom, the condensation reaction is carried out according to a conventional peptide synthesis technique, for example, an acid chloride method, an acid anhydride method, a mixed anhydride method, a method of using N,N′-dicyclohexylcarbodiimide (DCC), an active ester method, a method of using N,N′-carbonyldiimidazole (CDI), a method of using diethyl cyanophosphate (DEPC), a method of using N-ethyl-N′-(3-dimethylaminopropyl)carbodiimide hydrochloride (WSC.HCl) and 1-hydroxybenzotriazole (HOBt), or the like. Compound (VI) is used in an amount of about 1 to 2 mol, preferably about 1.0 to 1.1 mol, per 1 mol of compound (V). The reagent for the aforementioned methods is used in an amount of about 1 to 2 mol, preferably about 1.1 to 1.3 mol, per 1 mol of compound (V). The reaction temperature is generally −10 to 80° C., preferably 0 to 30° C.

When Y is an alkali metal atom, the condensation reaction is advantageously carried out according to a method using WSC HCl and HOBt. Compound (VI) is used in an amount of about 1 to 2 mol, preferably about 1.0 to 1.1 mol, per 1 mol of compound (V). WSC.HCl is used in an amount of about 1 to 4 mol, preferably about 1.5 to 2.5 mol, per 1 mol of compound (V). HOBt is used in an amount of about 1 to 8 mol, preferably about 2.5 to 5.0 mol, per 1 mol of compound (V). The reaction temperature is generally −10 to 100° C., preferably 40 to 70° C.

In any case, the condensation reaction is preferably carried out in a solvent. Examples of the solvent to be used include the above-mentioned halogenated hydrocarbons; the above-mentioned ethers; amides such as N,N-dimethylformamide, N,N-dimethylacetamide and the like; dimethyl sulfoxide, pyridine, acetonitrile and a mixed solvent thereof.

While the reaction time varies depending on the reagent or solvent to be used, it is generally 30 min to 3 days, preferably 30 min to 15 hr.

Compound (VII) can also be produced by further carrying out one or more of known hydrolysis reaction, acylation reaction, alkylation reaction, amination reaction, oxidation-reduction reaction, cyclization reaction, carbon chain extension reaction, substituent exchange reaction and the like, as desired.

Compound (I) can be produced by removing the N-protecting group PG of compound (VII). In addition, in each of the aforementioned reactions, when the starting compound has an amino group, a carboxyl group or a hydroxy group as a substituent, a protecting group generally used in peptide chemistry and the like may be introduced into these groups. By removing the protecting group as necessary after the reaction, the objective compound can be obtained. Introduction or removal of these protective groups may be carried out according to a method known per se, for example, the method disclosed in Theodora W. Greene and Peter G. M. Wuts, “Protective Groups in Organic Synthesis, 3^{rd} Ed.”, Wiley-Interscience (1999), or the like.

As the amino-protecting group, for example, formyl group; C₁₋₆ alkyl-carbonyl group, phenylcarbonyl group, C₁₋₆ alkoxy-carbonyl group, allyloxycarbonyl (Alloc) group, phenyloxycarbonyl group, fluorenylmethyloxycarbonyl (Fmoc) group, C₇₋₁₀ aralkyl-carbonyl group (e.g., benzylcarbonyl and the like), C₇₋₁₀ aralkyloxy-carbonyl group (e.g., benzyloxycarbonyl (Cbz) and the like), C₇₋₁₀ aralkyl group (e.g., benzyl and the like), trityl group, phthaloyl group, dithiasuccinoyl group, N,N-dimethylaminomethylene group, each optionally having substituent(s), and the like can be mentioned. As the substituent(s), for example, phenyl group, a halogen atom, C₁₋₆ alkyl-carbonyl group, C₁₋₆ alkoxy group optionally substituted by halogen atom(s) (e.g., methoxy, ethoxy, trifluoromethoxy and the like), nitro group and the like can be used. The number of the substituent(s) is 1 to 3.

As the carboxyl-protecting group, for example, C₁₋₆ alkyl group, allyl group, benzyl group, phenyl group, trityl group, trialkylsilyl group, each optionally having substituent(s), and the like can be mentioned. As the substituent(s), for example, a halogen atom, formyl group, C₁₋₆ alkyl-carbonyl group, C₁₋₆ alkoxy group optionally substituted by halogen atom(s) (e.g., methoxy, ethoxy, trifluoromethoxy and the like), nitro group and the like can be used. The number of the substituent(s) is 1 to 3.

As the hydroxy-protecting group, for example, C₁₋₆ alkyl group, C₇₋₂₀ aralkyl group (e.g., benzyl, trityl and the like), formyl group, C₁₋₆ alkyl-carbonyl group, benzoyl group, C₇₋₁₀ aralkyl-carbonyl group (e.g., benzylcarbonyl and the like), 2-tetrahydropyranyl group, tetrahydrofuranyl group, trialkylsilyl group (e.g., trimethylsilyl, tert-butyldimethylsilyl, diisopropylethylsilyl and the like), each optionally having substituent(s), and the like can be mentioned. As the substituent(s), for example, a halogen atom, C₁₋₆ alkyl group, phenyl group, C₇₋₁₀ aralkyl group (e.g., benzyl and the like), C₁₋₆ alkoxy group, nitro group and the like can be used. The number of the substituent(s) is 1 to 4.

When compound (I) is obtained as a free compound, it can be converted to the object salt according to a method known per se or a method analogous thereto, and when it is obtained as a salt, it can be converted to a free compound or the object salt according to a method known per se or a method analogous thereto.

Compound (I) may be used as a prodrug. A prodrug of compound (I) means a compound which is converted to compound (I) with a reaction due to an enzyme, an gastric acid, etc. under the physiological condition in the living body, that is, a compound which is converted to compound (I) with oxidation, reduction, hydrolysis, etc. according to an enzyme; a compound which is converted to compound (I) by hydrolysis etc. due to gastric acid, etc.

Examples of a prodrug of compound (I) include a compound wherein an amino group of compound (I) is acylated, alkylated or phosphorylated (e.g., compound wherein amino group of compound (I) is eicosanoylated, alanylated, pentylaminocarbonylated, (5-methyl-2-oxo-1,3-dioxolen-4-yl)methoxycarbonylated, tetrahydrofuranylated, pyrrolidylmethylated, pivaloyloxymethylated or tert-butylated, and the like); a compound wherein a hydroxy group of compound (I) is acylated, alkylated, phosphorylated or borated (e.g., a compound wherein a hydroxy group of compound (I) is acetylated, palmitoylated, propanoylated, pivaloylated, succinylated, fumarylated, alanylated or dimethylaminomethylcarbonylated, and the like); a compound wherein a carboxyl group of compound (I) is esterified or amidated (e.g., a compound wherein a carboxyl group of compound (I) is ethyl esterified, phenyl esterified, carboxymethyl esterified, dimethylaminomethyl esterified, pivaloyloxymethyl esterified, ethoxycarbonyloxyethyl esterified, phthalidyl esterified, (5-methyl-2-oxo-1,3-dioxolen-4-yl)methyl esterified, cyclohexyloxycarbonylethyl esterified or methylamidated, and the like) and the like. These compounds can be produced from compound (I) by a method known per se.

A prodrug of compound (I) may also be one which is converted into compound (I) under a physiological condition, such as those described in IYAKUHIN no KAIHATSU (Development of Pharmaceuticals), Vol. 7, Design of Molecules, p. 163-198, Published by HIROKAWA SHOTEN (1990).

When compound (I) has an isomer such as optical isomer, steric isomer, positional isomer, rotational isomer and the like, any isomers and a mixture thereof are encompassed in compound (I). For example, when compound (I) has an optical isomer, an optical isomer resolved from a racemate is also encompassed in compound (I). Such isomer can be obtained as a single product by a synthesis method, a separation method (e.g., concentration, solvent extraction, column chromatography, recrystallization etc.), optical resolution method (e.g., fractional recrystallization, chiral column method, diastereomer method etc.) and the like known per se.

Compound (I) may be a crystal, and both a single crystal and crystal mixtures are encompassed in compound (I). Crystals can be produced by crystallization according to crystallization methods known per se.

Compound (I) may be a solvate (e.g., hydrate etc.) or a non-solvate (e.g., non-hydrate etc.), both of which are encompassed in compound (I).

A compound labeled with an isotope (e.g., ³H, ¹⁴C, ³⁵S, ¹²⁵I and the like) and the like is also encompassed in compound (I).

Deuterium-converted compound wherein ¹H has been converted to ²H(D) are also encompassed in the compound (I).

Compound (I) or its prodrug, or salts thereof (hereinafter, sometimes to be abbreviated to as a compound of the present invention) exhibit superior renin inhibitory activity. They have low toxicity (e.g., acute toxicity, chronic toxicity, genetic toxicity, reproductive toxicity, cardiac toxicity, drug interaction, carcinogenicity, etc.) and high water-solubility, and are excellent in the aspects of stability, pharmacokinetics (absorbability, distribution, metabolism, excretion, etc.) and efficacy, thus being useful as medicine.

The compound of the present invention acts as a renin inhibitory drug in mammals (e.g., mouse, rat, hamster, rabbit, cat, dog, cattle, sheep, monkey, human, etc.), and is useful as a drug inhibiting the RA system by inhibiting the biosynthesis of AII, and is useful as an agent for the prophylaxis or treatment of various diseases caused by the RA system.

Examples of such diseases include hypertension (e.g., essential hypertension, renal vascular hypertension, renoparenchymal hypertension, primary aldosteronism, Cushing's syndrome etc.), blood pressure circadian rhythm abnormality, heart diseases (e.g., cardiac hypertrophy, acute heart failure, chronic heart failure including congestive heart failure, failure of expansion, cardiac myopathy, angina pectoris, myocarditis, atrial fibrillation, arrhythmia, tachycardia, cardiac infraction etc.), cerebrovascular disorders (e.g., asymptomatic cerebrovascular disorder, transient cerebral ischemia, apoplexy, cerebrovascular dementia, hypertensive encephalopathy, cerebral infarction etc.), cerebral edema, cerebral circulatory disorder, recurrence and sequela of cerebrovascular disorders (e.g., neurotic symptom, psychic symptom, subjective symptom, disorder in daily living activities etc.), ischemic peripheral circulation disorder, myocardial ischemia, venous insufficiency, progression of cardiac insufficiency after cardiac infarction, renal diseases (e.g., nephritis, glomerulonephritis, glomerulosclerosis, renal failure, nephrotic syndrome, thrombotic vasculopathy, complication of dialysis, organ damage including nephropathy by radiation irradiation etc.), arteriosclerosis including atherosclerosis (e.g., aneurysm, coronary arteriosclerosis, cerebral arteriosclerosis, peripheral arteriosclerosis etc.), vascular hypertrophy, vascular hypertrophy or obliteration and organ damages after intervention (e.g., percutaneous transluminal coronary angioplasty, stenting, coronary angioscopy, intravascular ultrasound, dounce thrombolytic therapy etc.), vascular re-obliteration and restenosis after bypass, polycythemia, hypertension, organ damage and vascular hypertrophy after transplantation, rejection after transplantation, ocular diseases (e.g., glaucoma, ocular hypertension etc.), thrombosis, multiple organ disorder, endothelial dysfunction, hypertensive tinnitus, other cardiovascular diseases (e.g., deep vein thrombosis, obstructive peripheral circulatory disorder, arteriosclerosis obliterans, obstructive thromboangiitis, ischemic cerebral circulatory disorder, Raynaud's disease, Berger disease etc.), metabolic and/or nutritional disorders (e.g., diabetes, impaired glucose tolerance, insulin resistance, hyperinsulinemia, diabetic nephropathy, diabetic retinopathy, diabetic neuropathy, obesity, hyperlipidemia, hypercholesterolemia, hyperuricacidemia, hyperkalemia, hypernatremia etc.), metabolic syndrome, nerve degeneration diseases (e.g., Alzheimer's disease, Parkinson's syndrome, Creutzfeldt-Jakob disease, multiple sclerosis, amyotrophic lateral sclerosis, AIDS encephalopathy etc.), central nervous system disorders (e.g., damages such as cerebral hemorrhage and cerebral infarction, and sequela and complication thereof, head injury, spinal injury, cerebral edema, sensory malfunction, sensory functional disorder, autonomic nervous system disorder, autonomic nervous system malfunction etc.), dementia, migraine, defects of memory, disorder of consciousness, amnesia, anxiety symptom, catatonic symptom, discomfort mental state, sleep disorder, agrypnia, sychopathies (e.g., depression, epilepsy, alcoholism etc.), inflammatory diseases (e.g., arthritis such as rheumatoid arthritis, osteoarthritis, rheumatoid myelitis, periostitis etc.; inflammation after operation or injury; remission of swelling; pharyngitis; cystitis; pneumonia; atopic dermatitis; inflammatory intestinal diseases such as Crohn's disease, ulcerative colitis etc.; meningitis; inflammatory ocular disease; inflammatory pulmonary disease such as pneumonia, pulmonary silicosis, pulmonary sarcoidosis, pulmonary tuberculosis etc.), allergic diseases (e.g., allergic rhinitis, conjunctivitis, gastrointestinal allergy, pollinosis, anaphylaxis etc.), chronic obstructive pulmonary disease, interstitial pneumonia, pneumocytis carinni pneumonia, collagen diseases (e.g., systemic lupus erythematodes, scleroderma, polyarteritis etc.), hepatic diseases (e.g., hepatitis including chronic hepatitis, hepatic cirrhosis etc.), portal hypertension, digestive system disorders (e.g., gastritis, gastric ulcer, gastric cancer, gastric disorder after operation, dyspepsia, esophageal ulcer, pancreatitis, colon polyp, cholelithiasis, hemorrhoidal disease, varices ruptures of esophagus and stomach etc.), blood and/or myelopoietic diseases (e.g., erythrocytosis, vascular purpura, autoimmune hemolytic anemia, disseminated intravascular coagulation syndrome, multiple myelopathy etc.), bone diseases (e.g., fracture, refracture, osteoporosis, osteomalacia, bone Paget's disease, sclerosing myelitis, rheumatoid arthritis, joint tissue dysfunction and the like caused by osteoarthritis of the knee and diseases similar to these), solid tumor, tumors (e.g., malignant melanoma, malignant lymphoma, cancer of digestive organs (e.g., stomach, intestine etc.) etc.), cancer and cachexia following cancer, metastasis cancer, endocrinopathy (e.g., Addison's disease, pheochromocytoma etc.), urinary organ and/or male genital diseases (e.g., cystitis, prostatic hypertrophy, prostatic cancer, sex infectious disease etc.), female disorders (e.g., climacteric disorder, gestosis, endometriosis, hysteromyoma, ovarian disease, breast disease, sex infectious disease etc.), disease relating to environment and occupational factors (e.g., radiation hazard, hazard by ultraviolet, infrared or laser beam, altitude sickness etc.), respiratory diseases (e.g., cold syndrome, pneumonia, asthma, pulmonary hypertension, pulmonary thrombosis and pulmonary embolism etc.), infectious diseases (e.g., viral infectious diseases with cytomegalovirus, influenza virus, herpes virus etc., rickettsiosis, bacterial infectious disease etc.), toxemias (e.g., sepsis, septic shock, endotoxin shock, Gram-negative sepsis, toxic shock syndrome etc.), otorhinolaryngological diseases (e.g., Meniere's syndrome, tinnitus, dysgeusia, vertigo, disequilibrium, dysphagia etc.), skin diseases (e.g., keloid, hemangioma, psoriasis etc.), intradialytic hypotension, myasthenia gravis, systemic diseases such as chronic fatigue syndrome and the like.

The compound of the present invention can be used in combination with an existing hypertension therapeutic drug such as an ACE inhibitor (captopril, enalapril maleate, alacepril, delapril hydrochloride, imidapril hydrochloride, quinapril hydrochloride, cilazapril, temocapril hydrochloride, trandolapril, benazepril hydrochloride, perindopril, lisinopril, etc.), ARB (losartan potassium, candesartan cilexetil, valsartan, TAK-536, TAK-491, irbesartan, telmisartan, eprosartan, olmesartan medoxomil, etc.), an aldosterone receptor antagonist (spironolactone, eplerenone, etc.), a Ca-ion channel inhibitor (verapamil hydrochloride, diltiazem hydrochloride, nifedipine, amlodipine hydrochloride, azelnidipine, aranidipine, efonidipine hydrochloride, cilnidipine, nicardipine hydrochloride, nisoldipine, nitrendipine, nilvadipine, barnidipine hydrochloride, felodipine, benidipine hydrochloride, manidipine hydrochloride, etc.), diuretic (trichlormethiazide, hydrochlorothiazide, benzylhydrochlorothiazide, indapamide, tripamide, meticrane, mefruside, furosemide, triamterene, chlorthalidon etc.), a β-blocker (propranolol hydrochloride, atenolol, metoprolol tartrate, bisoprolol fumarate, etc.), an α,β-blocker (carvedilol, etc.), and the like.

Moreover, the compound of the present invention can be also used in combination with an antithrombotic drug such as heparin sodium, heparin calcium, warfarin calcium (Warfarin), a blood coagulation factor Xa inhibitor, drug having a function of balance correction in the coagulation-fibrinolysis system, an oral thrombin inhibitor, a thrombolytic drug (tPA, urokinase, etc.), an antiplatelet drug [aspirin, sulfinpyrazone (Anturane), dipyridamol (Persantine), ticlopidine hydrochloride (Panaldine), clopidogrel, cilostazol (Pletal), GPIIb/IIIa antagonist (ReoPro, etc.)], and the like. Also, the compound can be used in combination with a lipid lowering drug or a cholesterol lowering drug. Examples thereof include a squalene synthase inhibitor (lapaquistat acetate etc.), fibrates (clofibrate, benzafibrate, gemfibrozil, etc.), nicotinic acid, its derivatives and analogs (acipimox, probucol, etc.), a bile acid binding resin (cholestyramine, colestipol, etc.), an omega-3 polyunsaturated fatty acid (EPA (eicosapentaenoic acid), DHA (docosahexaenoic acid), or a mixture thereof etc.), a compound inhibiting cholesterol absorption (sitosterol, neomycin, etc.), and a squalene epoxidase inhibitor (NB-598 and its analogs, etc.). Furthermore, other possible combination components are an oxidosqualene-lanosterol cyclase, for example, a decalin derivative, an azadecalin derivative, an indane derivative and the like. Combination with a HMG-CoA reductase (3-hydroxy-3-methylglutaryl coenzyme A reductase) inhibitor (atorvastatin calcium hydrate, pravastatin sodium, simvastatin, itavastatin, lovastatin, fluvastatin, etc.) is also possible.

The compound of the present invention can also be used in combination with a therapeutic drug for diabetes or a therapeutic drug for diabetic complications. For example, the compound of the present invention can be used in combination with an insulin preparation, an insulin sensitivity improving drug [pioglitazone hydrochloride, rosiglitazone, etc.], an α-glucosidase inhibitor [voglibose, acarbose, miglitol, emiglitate etc.], biguanide [phenformin, metformin, buformine etc.], insulin secretagogue [tolbutamide, glibenclamide, gliclazide, nateglinide, mitiglinide, glimepiride etc.], a dipeptidylpeptidase IV inhibitor [Alogliptin benzoate, Vidagliptin (LAF237), P32/98, Saxagliptin (BMS-477118) etc.], Kinedak, Penfill, Humulin, Euglucon, Glimicron, Daonil, Novolin, Monotard, Glucobay, Dimelin, Rastinon, Bacilcon, Deamelin S, Iszilin family, or the like.

In addition, the compound can be also used together with other pharmaceutical components, including a bone disease medicine, a myocardial protective drug, a coronary artery disease medicine, a chronic cardiac failure medicine, a hypothyroidism medicine, a nephrotic syndrome medicine, a chronic renal failure medicine, a gynecological disease medicine, an infection medicine, or the like.

The administration mode may be exemplified by (1) administration of a single preparation obtained by simultaneously formulating the compound of the present invention and the combination drug, (2) simultaneous administration through the same administration route of two preparations obtained by separately formulating the compound of the present invention and the combination drug, (3) administration with a time interval through the same administration route of two preparations obtained by separately formulating the compound of the present invention and the combination drug, (4) simultaneous administration through different administration routes of two preparations obtained by separately formulating the compound of the present invention and the combination drug, (5) administration with a time interval through different administration routes of two preparations obtained by separately formulating the compound of the present invention and the combination drug (for example, administration in order of the compound of the present invention and then the combination drug, or administration in the reverse order), or the like. The amount of the combination drug to be administered can be appropriately selected with reference to the clinically used dosage. The mixing ratio of the compound of the present invention and the combination drug can be appropriately selected in accordance with the subject of administration, administration route, disease to be treated, symptoms, combination, and the like.

The compound of the present invention can be also used in combination with, for example, gene therapy involving VEGF, TNFα or the like, or therapeutic methods involving various antibody medicines or the like.

The compound of the present invention can be safely administered individually, or according to ordinary methods (for example, methods described in the Japanese Pharmacopeia, etc.), as a pharmaceutical composition mixed with pharmaceutically acceptable carriers, for example, a tablet (including a sugar-coated tablet and a film-coated tablet), a film, a powder, a granule, a capsule, a liquid, an emulsion, a suspension, an injectable preparation, a suppository, a sustained release preparation, a patch and the like, either orally or parenterally (e.g., topical, rectal, intravenous administration, etc.).

The dosage form of the aforementioned pharmaceutical preparation may be exemplified by oral preparations such as a tablet (including a sublingual tablet and a buccal disintegration tablet), a film (including a buccal disintegration film), a capsule (including a soft capsule and a microcapsule), a granule, a powder, a troche, a syrup, an emulsion, a suspension and the like; and parenteral preparations such as an injectable preparation (e.g., a subcutaneous injectable preparation, an intravenous injectable preparation, intramuscular injectable preparation, intraperitoneal injectable preparation, a drip infusion), external preparation (e.g., a percutaneous preparation, an ointment), a suppository (e.g., a rectal suppository, a vaginal suppository), a pellet, a transnasal preparation, a transpulmonary preparation (inhalant), an eye drop and the like.

These preparations may be controlled release preparations such as a rapid release preparation, a sustained release preparation and the like (e.g., a sustained release microcapsule).

The content of the compound of the present invention in the pharmaceutical composition is about 0.01 to 100% by weight of the entire composition.

The amount of administration of the compound of the present invention may vary depending on the subject of administration, administration route, subject disease or the like; however, in the case of administering orally to an adult as a hypertension medicine, the amount of administration is about 0.0005 to 2 mg/kg of body weight, preferably about 0.001 to 1 mg/kg of body weight, and more preferably about 0.001 to 0.5 mg/kg of body weight, in terms of compound (I), the active ingredient, possibly once to several times a day.

The aforementioned pharmaceutically acceptable carrier may be exemplified by various organic or inorganic carrier materials that are conventionally used as preparation materials, for example, excipient, gliding agent, binding agent and disintegrant for solid preparations; or solvent, solution aid, suspending agent, isotonic agent, buffering agent, soothing agent and the like for liquid preparations. Further, if necessary, additives such as preservative, antioxidant, colorant, sweetening agent, adsorbing agent, wetting agent and the like can be also used.

Examples of the excipient include lactose, white sugar, D-mannitol, starch, corn starch, crystalline cellulose, light silicic anhydride and the like.

Examples of the gliding agent include magnesium stearate, calcium stearate, talc, colloidal silica and the like.

Examples of the binding agent include crystalline cellulose, white sugar, D-mannitol, dextrin, hydroxypropylcellulose, hydroxypropylmethylcellulose, polyvinylpyrrolidone, starch, sucrose, gelatin, methylcellulose, carboxymethylcellulose sodium and the like.

Examples of the disintegrant include starch, carboxymethylcellulose, carboxymethylcellulose calcium, carboxymethylstarch sodium, L-hydroxypropylcellulose and the like.

Examples of the solvent include water for injection, alcohol, propylene glycol, Macrogol, sesame oil, corn oil, olive oil and the like.

Examples of the dissolution aid include polyethylene glycol, propylene glycol, D-mannitol, benzyl benzoate, ethanol, trisaminomethane, cholesterol, triethanolamine, sodium carbonate, sodium citrate and the like.

Examples of the suspending agent include surfactants such as stearyl triethanolamine, sodium lauryl sulfate, laurylaminopropionic acid, lecithin, benzalkonium chloride, benzetonium chloride, glycerin monostearate and the like; hydrophilic polymers such as polyvinyl alcohol, polyvinylpyrrolidone, carboxymethylcellulose sodium, methylcellulose, hydroxymethylcellulose, hydroxyethylcellulose, hydroxypropylcellulose and the like; and the like.

Examples of the isotonic agent include glucose, D-sorbitol, sodium chloride, glycerin, D-mannitol and the like.

Examples of the buffering agent include buffer solutions such as phosphates, acetates, carbonates, citrates and the like.

Examples of the soothing agent include benzyl alcohol and the like.

Examples of the preservative include parahydroxybenzoic acid esters, chlorobutanol, benzyl alcohol, phenethyl alcohol, dehydroacetic acid, sorbic acid and the like.

Examples of the antioxidant include sulfites, ascorbic acid, α-tocopherol and the like.

Examples of the colorant include water-soluble Food coal tar dyes (e.g., Food dyes such as Food Red No. 2 and No. 3, Food Yellow No. 4 and No. 5, Food Blue No. 1 and No. 2, and the like), water-insoluble lake dyes (e.g., aluminum salts of the aforementioned water-soluble Food coal tar dyes), natural dyes (e.g., β-carotene, chlorophyll, red iron oxide) and the like.

Examples of the sweetening agent include saccharin sodium, dipotassium glycyrrhizinate, aspartame, stevia and the like.

### EXAMPLES

The present invention is explained in detail in the following by referring to Reference Examples, Examples, Preparation Examples and Experimental Examples, which are not to be construed as limitative. Of the synthesis starting materials used in Reference Examples and Examples, synthetic methods of known compounds are omitted.

“Room temperature” in the following Reference Examples and Examples represents a temperature of about 10° C. to about 35° C., and “%” represents weight % unless otherwise stated. Provided that, yield represents mol/mol %.

¹H-NMR spectra were measured with a Varian MERCURY 300 (300 MHz) spectrometer or a BRUKER ADVANCE 300 spectrometer (300 MHz) using tetramethylsilane as an internal standard. All of the δ values are represented in ppm.

LC/MS spectra were measured under the following conditions.

Equipment: Agilent 1100 HPLC (Gilson 215 autosampler)/Waters ZQ, or Waters 2795/ZQ
Column: CapcellPak C18UG120 (1.5 mmID×35 mL, S-3 μm), manufactured by Shiseido Co., Ltd.
Solvent: Solution A (0.05% trifluoroacetic acid-containing water), Solution B (0.04% trifluoroacetic acid-containing water)
Gradient cycle: 0.00 min (A/B=90/10), 2.00 min (A/B=5/95), 2.75 min (A/B=5/95), 2.76 min (A/B=90/10), 3.45 min (A/B=90/10)
Flow rate: 0.5 ml/min

### Detection: UV (220 nm)

Mass spectrum: electrospray ionization (ESI)

Reverse-phase HPLC analysis was carried out on an YMC CombiPrep ODS-A (20 mmID×50 mL, S-5 μm) Column using a Gilson UniPoint system, and eluted with 0.1% trifluoroacetic acid-containing acetonitrile/water (10:90-100:0) at a flow rate of 25 ml/min.

The microwave reactor used was Discover of CEM.

Other symbols used in the present text indicate the following meanings.

s: singlet, d: doublet, t: triplet, q: quartet, dd: double doublet, dt: double triplet, td: triple doublet, dq: double quartet, tq: triple quartet, ddd: double double doublet, m: multiplet, br: broad.
Me: methyl, Et: ethyl, nPr: n-propyl, iPr: isopropyl, nBu: n-butyl, iBu: isobutyl, ^{t}Bu: tert-butyl, Boc: tert-butoxycarbonyl, Cbz: benzyloxycarbonyl, Tr: trityl.
DMA: N,N-dimethylacetamide, DME: 1,2-dimethoxyethane, DMF: N,N-dimethylformamide, DMSO: dimethyl sulfoxide, THF: tetrahydrofuran.
ADDP: 1,1′-(azodicarbonyl)dipiperidine,
9-BBN: 9-borabicyclo[3.3.1]nonane,
BEMP: 2-tert-butylimino-2-diethylamino-1,3-dimethylperhydro-1,3,2-diazaphosphorin,
BINAP: 2,2′-bis(diphenylphosphino)-1,1′-binaphthyl,
DAST: (diethylamino)sulfur trifluoride,
DBU: 1,8-diazabicyclo[5.4.0]-7-undecene,
DCC: dicyclohexylcarbodiimide,
DEAD: diethyl azodicarboxylate,
DMAP: 4-(dimethylamino)pyridine,
dppf: 1,1′-bis(diphenylphosphino)ferrocene,
DTBAD: di-tert-butyl azodicarboxylate,
HATU: O-(7-azabenzotriazol-1-yl)-N,N,N′,N′-tetramethyluronium hexafluorophosphate,
HOBt: 1-hydroxybenzotriazole,
mCPBA: m-chloroperbenzoic acid,

### NBS: N-bromosuccinimide,

Pd₂ (dba) 3: tris(dibenzylideneacetone)dipalladium(0),
TBAF: tetra-n-butylammonium fluoride,
TFA: trifluoroacetic acid,
WSC.HCl: 1-ethyl-3-[3-(dimethylamino)propyl]carbodiimide hydrochloride.

### Reference Example 1

### Ethyl 2-(formylamino)-3-phenylacrylate

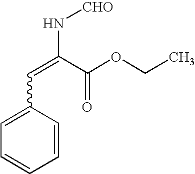

Sodium hydride (60% in oil) (11.62 g) was suspended in THF (270 ml), and, while stirring the suspension, a solution of benzaldehyde (28.27 g) and ethyl isocyanoacetate (27.39 g) in THF (55 ml) was added dropwise over 20 min at room temperature. The mixture was stirred at room temperature for 2.5 hr, and ice-cooled. Acetic acid (45 ml) was added dropwise, and the mixture was stirred for 10 min, poured into ice water, and extracted with ethyl acetate. The extract was washed successively with water, saturated aqueous sodium hydrogen carbonate, water and saturated brine, and dried over anhydrous magnesium sulfate, and the solvent was evaporated under reduced pressure. The residue was subjected to silica gel column chromatography, and the fraction eluted with ethyl acetate-hexane (1:2-2:1) was concentrated under reduced pressure to give the object compound (40.27 g) as an oil.

¹H-NMR (CDCl₃) δ 0.98-1.40 (3H, m), 4.06-4.38 (2H, m), 7.06-7.68 (7H, m), 8.21-8.47 (1H, m)

### Reference Example 2

### Ethyl 3-bromo-2-(formylamino)-3-phenylacrylate

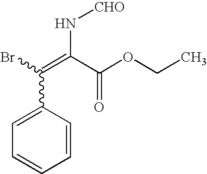

Ethyl 2-(formylamino)-3-phenylacrylate (40.27 g) was dissolved in carbon tetrachloride-chloroform (3:1, 440 ml), the solution was ice-cooled, and NBS (34.33 g) was added. The mixture was stirred at 0° C. for 1.5 hr, and then at room temperature for 3 hr, and ice-cooled again. Triethylamine (19.52 g) was added, and the mixture was stirred at 0° C. for 20 min, and then at room temperature for 40 min. The reaction mixture was washed successively with water and saturated brine, and dried over anhydrous magnesium sulfate, and the solvent was evaporated under reduced pressure. The residue was subjected to silica gel column chromatography, and the fraction eluted with ethyl acetate-hexane (1:3-1:2) was concentrated under reduced pressure to give the object compound (44.88 g) as an oil.

¹H-NMR (CDCl₃) δ 0.89-1.45 (3H, m), 3.97-4.46 (2H, m), 6.91 (1H, br s), 7.28-7.46 (5H, m), 7.95-8.28 (1H, m)

### Reference Example 3

### Ethyl 3-bromo-2-isocyano-3-phenylacrylate

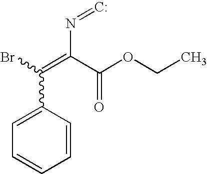

Ethyl 3-bromo-2-(formylamino)-3-phenylacrylate (16.33 g) and triethylamine (13.86 g) were dissolved in dichloromethane (150 ml), and the solution was ice-cooled. Phosphoryl chloride (9.24 g) was added, and the mixture was stirred at 0° C. for 2 hr. The reaction mixture was poured into saturated aqueous sodium hydrogen carbonate, and the mixture was vigorously stirred at room temperature for 1 hr, and extracted with ethyl acetate. The extract was washed successively with water and saturated brine, and dried over anhydrous magnesium sulfate. The solvent was evaporated under reduced pressure, and the residue was subjected to silica gel column chromatography. The fraction eluted with ethyl acetate-hexane (1:6) was concentrated under reduced pressure at 30° C. or below to give the object compound (14.82 g) as an oil.

¹H-NMR (CDCl₃) δ 1.03-1.42 (3H, m), 4.04-4.42 (2H, m), 7.25-7.56 (5H, m)

### Reference Example 4

### Methyl 1-cyclohexyl-5-phenyl-1H-imidazole-4-carboxylate

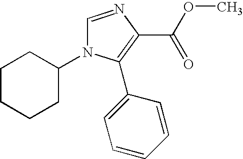

Cyclohexylamine (0.21 ml) and triethylamine (0.26 ml) were dissolved in DMF (5 ml), and the solution was ice-cooled. Methyl 3-bromo-2-isocyano-3-phenylacrylate (500 mg) was added, and the mixture was stirred at room temperature for 12 hr. The reaction mixture was concentrated under reduced pressure, and partitioned between ethyl acetate and water. The organic layer was washed with saturated brine, and dried over anhydrous sodium sulfate, and the solvent was evaporated under reduced pressure. The residue was subjected to silica gel column chromatography, and the fraction eluted with ethyl acetate was concentrated under reduced pressure to give the object compound (240 mg).

MS (ESI+, m/e) 285 (M+1)

### Reference Example 5

### Methyl 1-[(1S,2S)-2-(benzyloxy)cyclohexyl]-5-phenyl-1H-imidazole-4-carboxylate

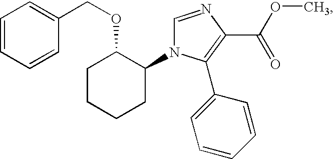

(1S,2S)-2-(Benzyloxy)cyclohexylamine (848 mg) and triethylamine (1.06 ml) were dissolved in DMF (10 ml), and the solution was ice-cooled. Methyl 3-bromo-2-isocyano-3-phenylacrylate (1.0 g) was added, and the mixture was stirred at room temperature for 12 hr. The reaction mixture was concentrated under reduced pressure, and partitioned between ethyl acetate and water. The organic layer was washed with saturated brine, and dried over anhydrous sodium sulfate, and the solvent was evaporated under reduced pressure. The residue was subjected to silica gel column chromatography, and the fraction eluted with ethyl acetate was concentrated under reduced pressure to give the object compound (1.26 g).

¹H-NMR (CDCl₃) δ 1.05-1.38 (3H, m), 1.56-1.84 (3H, m), 1.91-2.01 (1H, m), 2.17-2.30 (1H, m), 3.44-3.59 (1H, m), 3.68-3.78 (1H, m), 3.79 (3H, s), 4.25 (1H, d), 4.43 (1H, d), 6.99-7.09 (2H, m), 7.22-7.33 (3H, m), 7.33-7.48 (5H, m), 7.57 (1H, s)

In the same manner as in Reference Example 5, the following compounds (Reference Examples 6-14) were obtained.

### Reference Example 6

### Methyl 1-[(1R,2R)-2-(benzyloxy)cyclohexyl]-5-phenyl-1H-imidazole-4-carboxylate

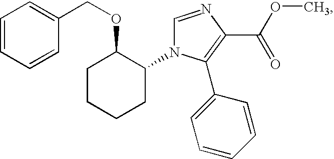

¹H-NMR (CDCl₃) δ 1.05-1.38 (3H, m), 1.56-1.84 (3H, m), 1.91-2.01 (1H, m), 2.17-2.30 (1H, m), 3.44-3.59 (1H, m), 3.68-3.78 (1H, m), 3.79 (3H, s), 4.25 (1H, d), 4.43 (1H, d), 6.99-7.09 (2H, m), 7.22-7.33 (3H, m), 7.33-7.48 (5H, m), 7.57 (1H, s)

### Reference Example 7

### Methyl 1-(trans-4-hydroxycyclohexyl)-5-phenyl-1H-imidazole-4-carboxylate

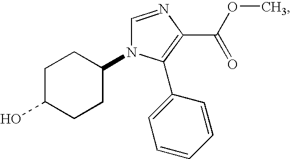

¹H-NMR (CDCl₃) δ 1.17-1.36 (2H, m), 1.68-1.87 (2H, m), 1.96-2.09 (4H, m), 3.65-3.79 (5H, m), 7.28-7.37 (2H, m), 7.45-7.55 (3H, m), 7.64 (1H, s)

### Reference Example 8

### Methyl 1-cyclopentyl-5-phenyl-1H-imidazole-4-carboxylate

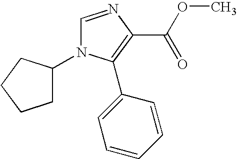

¹H-NMR (CDCl₃) δ 1.53-1.70 (2H, m), 1.75-1.91 (4H, m), 1.96-2.15 (2H, m), 3.77 (3H, s), 4.18-4.29 (1H, m), 7.29-7.39 (2H, m), 7.43-7.52 (2H, m), 7.65 (1H, s)

### Reference Example 9

### Methyl 1-cycloheptyl-5-phenyl-1H-imidazole-4-carboxylate

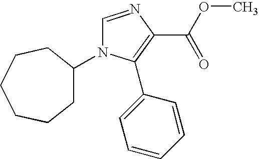

¹H-NMR (CDCl₃) δ 1.26-1.40 (2H, m), 1.49-1.63 (4H, m), 1.64-1.82 (2H, m), 1.83-1.93 (2H, m), 1.95-2.05 (2H, m), 3.77 (3H, s), 3.81-3.93 (1H, m), 7.32 (2H, s), 7.43-7.53 (3H, m), 7.66 (1H, s)

### Reference Example 10

### Methyl 1-(trans-2-hydroxycyclopentyl)-5-phenyl-1H-imidazole-4-carboxylate

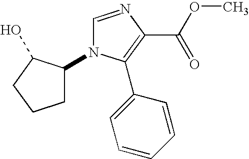

¹H-NMR (CDCl₃) δ 1.52-1.71 (2H, m), 1.73-1.86 (3H, m), 2.04-2.22 (2H, m), 2.62 (1H, br s), 3.75 (3H, s), 4.10 (1H, s), 7.34-7.42 (2H, m), 7.44-7.52 (3H, m), 7.61 (1H, s)

### Reference Example 11

### Methyl 1-[(1R,2R)-2-(benzyloxy)cyclopentyl]-5-phenyl-1H-imidazole-4-carboxylate

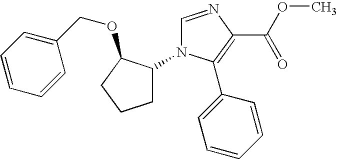

¹H-NMR (CDCl₃) δ 1.67-1.87 (4H, m), 1.95-2.09 (2H, m), 2.13-2.21 (1H, m), 3.78 (3H, s), 4.03-4.09 (1H, m), 4.22-4.37 (2H, m), 7.12-7.15 (2H, m), 7.26-7.47 (7H, m), 7.57 (1H, s)

MS (ESI+, m/e) 377 (M+1)

### Reference Example 12

### Methyl 1-[(2R)-bicyclo[2.2.1]hept-2-yl]-5-phenyl-1H-imidazole-4-carboxylate

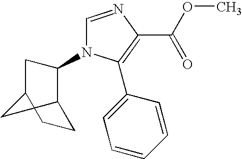

¹H-NMR (CDCl₃) δ 1.04-1.09 (2H, m), 1.36-1.74 (5H, m), 1.83-1.93 (1H, m), 2.41-2.49 (2H, m), 3.76 (3H, s), 3.76-3.82 (1H, m), 7.32-7.35 (2H, m), 7.46-7.49 (3H, m), 7.69 (1H, s)

MS (ESI+, m/e) 297 (M+1)

### Reference Example 13

### Methyl 1-[bicyclo[2.2.1]hept-2-yl]-5-phenyl-1H-imidazole-4-carboxylate

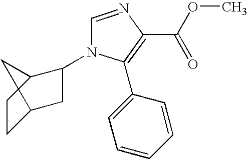

¹H-NMR (CDCl₃) δ 1.31-1.45 (4H, m), 1.51-1.57 (1H, m), 1.63-1.72 (1H, m), 1.99-2.13 (2H, m), 2.33-2.36 (1H, m), 3.76 (3H, s), 4.24-4.31 (1H, m), 7.31-7.35 (2H, m), 7.45-7.48 (2H, m), 7.67 (1H, s)

MS (ESI+, m/e) 297 (M+1)

### Reference Example 14

### Methyl 1-[cis-2-(hydroxymethyl)cyclohexyl]-5-phenyl-1H-imidazole-4-carboxylate

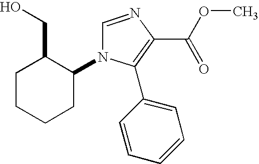

¹H-NMR (CDCl₃) δ 1.37-1.51 (2H, m), 1.51-1.65 (2H, m), 1.65-1.79 (3H, m), 1.86 (2H, dd), 3.44 (1H, d), 3.57 (1H, s), 3.75 (3H, s), 4.14-4.24 (2H, m), 7.32-7.41 (2H, m), 7.41-7.54 (3H, m), 7.71 (1H, s)

### Reference Example 15

### Ethyl 1-[(1S,2R)-2-hydroxycyclohexyl]-5-phenyl-1H-imidazole-4-carboxylate

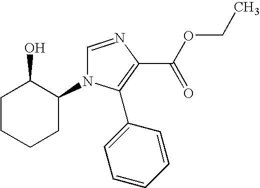

(1R,2S)-2-Aminocyclohexanol hydrochloride (5.3 g) and triethylamine (15.1 ml) were dissolved in DMF (100 ml), and the solution was ice-cooled. Ethyl 3-bromo-2-isocyano-3-phenylacrylate (9.8 g) was added, and the mixture was stirred at room temperature for 12 hr. The reaction mixture was concentrated under reduced pressure, and partitioned between ethyl acetate and water. The organic layer was washed with saturated brine, and dried over anhydrous sodium sulfate, and the solvent was evaporated under reduced pressure. The residue was subjected to silica gel column chromatography, and the fraction eluted with ethyl acetate-methanol (9:1) was concentrated under reduced pressure to give the object compound (6.13 g).

¹H-NMR (CDCl₃) δ 1.10 (3H, t), 1.21-1.37 (2H, m), 1.38-1.52 (1H, m), 1.60-1.79 (2H, m), 1.80-1.97 (2H, m), 2.22-2.37 (2H, m), 3.76 (1H, dt), 4.04 (1H, br s), 4.13 (2H, q), 7.20-7.31 (2H, m), 7.39-7.51 (3H, m), 7.86 (1H, s)

In the same manner as in Reference Example 15, the following compounds (Reference Examples 16-20) were obtained.

### Reference Example 16

### Ethyl 1-[(1S,2S)-2-hydroxycyclohexyl]-5-phenyl-1H-imidazole-4-carboxylate

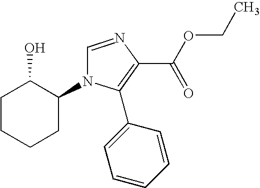

MS (ESI+, m/e) 315 (M+1)

### Reference Example 17

### Ethyl 1-(cis-2-hydroxycyclohexyl)-5-phenyl-1H-imidazole-4-carboxylate

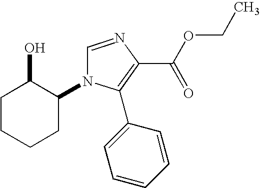

¹H-NMR (CDCl₃) δ 1.10 (3H, t), 1.21-1.37 (2H, m), 1.38-1.52 (1H, m), 1.60-1.79 (2H, m), 1.80-1.97 (2H, m), 2.22-2.37 (2H, m), 3.76 (1H, dt), 4.04 (1H, br s), 4.13 (2H, q), 7.20-7.31 (2H, m), 7.39-7.51 (3H, m), 7.86 (1H, s)

### Reference Example 18

### Ethyl 1-[(1S)-1-(1-hydroxycyclohexyl)ethyl]-5-phenyl-1H-imidazole-4-carboxylate

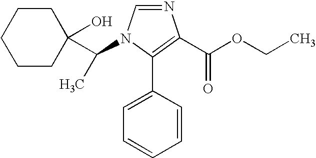

¹H-NMR (CDCl₃) δ 0.99-1.14 (3H, m), 1.17-1.26 (3H, m), 1.29-1.37 (2H, m), 1.43-1.58 (5H, m), 1.61-1.68 (5H, m), 3.81 (1H, q), 4.22 (2H, dq), 7.47 (3H, td), 7.96 (1H, s)

### Reference Example 19

### Ethyl 1-[(S)-(1-hydroxycyclohexyl)(phenyl)methyl]-5-phenyl-1H-imidazole-4-carboxylate

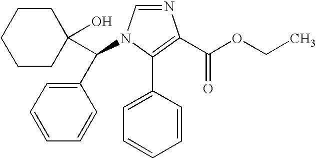

¹H-NMR (CDCl₃) δ 1.19 (3H, t), 1.33-1.47 (3H, m), 1.49-1.65 (7H, m), 4.19 (2H, dd), 4.64 (1H, s), 7.10-7.24 (3H, m), 7.31-7.40 (4H, m), 7.46 (3H, s), 8.57 (1H, s)

### Reference Example 20

### Ethyl 1-[(R)-(1-hydroxycyclohexyl)(phenyl)methyl]-5-phenyl-1H-imidazole-4-carboxylate

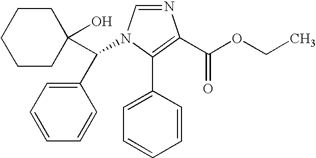

¹H-NMR (CDCl₃) δ 1.20 (3H, t), 1.35-1.47 (3H, m), 1.49-1.60 (3H, m), 1.68 (4H, d), 4.19 (2H, dq), 4.64 (1H, s), 7.21 (3H, dd), 7.31-7.36 (4H, m), 7.40-7.49 (3H, m), 8.57 (1H, s)

### Reference Example 21

### Ethyl 1-[trans-2-hydroxycycloheptyl]-5-phenyl-1H-imidazole-4-carboxylate

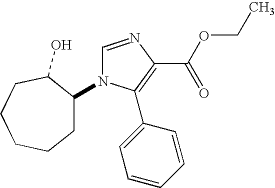

A mixture of ethyl 3-bromo-2-isocyano-3-phenylacrylate (1.50 g), trans-2-aminocycloheptanol (1.05 g), triethylamine (4.50 ml) and DMF (20 ml) was stirred at room temperature for 2 days, and concentrated under reduced pressure. The residue was dissolved in ethyl acetate, and the solution was washed successively with water and saturated brine, and dried over anhydrous magnesium sulfate. The solvent was evaporated under reduced pressure. The residue was subjected to silica gel column chromatography, and the fraction eluted with ethyl acetate-methanol (1:0-9:1) was concentrated under reduced pressure to give the object compound (860 mg).

¹H-NMR (CDCl₃) δ 1.21 (3H, t), 1.27-1.41 (1H, m), 1.51-1.61 (3H, m), 1.63-1.72 (3H, m), 1.77-1.84 (2H, m), 1.88-2.01 (1H, m), 3.74-3.86 (1H, m), 3.93-4.04 (1H, m), 4.19 (2H, q), 7.36-7.49 (5H, m), 7.61 (1H, s)

MS (ESI+, m/e) 329 (M+1)

### Reference Example 22

### Ethyl 1-[(1-hydroxycyclohexyl)methyl]-5-phenyl-1H-imidazole-4-carboxylate

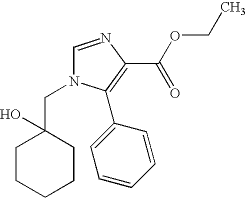

A mixture of ethyl 3-bromo-2-isocyano-3-phenylacrylate (500 mg), 1-(aminomethyl)cyclohexanol (440 mg), N,N-diisopropylethylamine (1.9 ml) and DMF (7 ml) was stirred at room temperature for 12 hr, poured into water, and the mixture was extracted with ethyl acetate. The extract was washed with saturated brine, and dried over anhydrous magnesium sulfate, and the solvent was evaporated under reduced pressure. The residue was subjected to basic silica gel column chromatography, and the fraction eluted with ethyl acetate-hexane (1:9-1:0) was concentrated under reduced pressure to give the object compound (447 mg).

¹H-NMR (CDCl₃) δ 1.02-1.17 (3H, m), 1.23 (3H, t), 1.28-1.37 (4H, m), 1.44-1.47 (1H, m), 1.63 (3H, br s), 3.80 (2H, s), 4.19 (2H, q), 7.28-7.37 (2H, m), 7.39-7.50 (3H, m), 7.79 (1H, s)

MS (ESI+, m/e) 329 (M+1)

### Reference Example 23

### Ethyl 1-{(1S,2S)-2-[(tert-butoxycarbonyl)amino]cyclohexyl}-5-phenyl-1H-imidazole-4-carboxylate

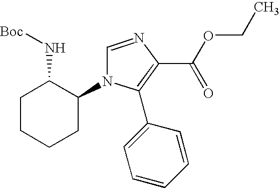

tert-Butyl [(1S,2S)-2-aminocyclohexyl]carbamate (1.29 g) and ethyl 3-bromo-2-isocyano-3-phenylacrylate (1.4 g) were dissolved in DMF (15 ml). N,N-Diisopropylethylamine (1.29 g) was added, and the mixture was stirred at room temperature for 40 hr. DBU (761 mg) was added to the reaction mixture, and the mixture was further stirred at room temperature for 1 hr. Saturated brine was added to the reaction mixture, and the liberated oil was extracted with ethyl acetate. The extract was washed successively with 6% aqueous sodium bicarbonate, 10% aqueous citric acid solution and saturated brine, dried over anhydrous magnesium sulfate, and concentrated under reduced pressure. The residue was subjected to basic silica gel column chromatography, and the fraction eluted with ethyl acetate-hexane (1:1-1:0) was concentrated under reduced pressure to give the object compound (1.24 g).

¹H-NMR (CDCl₃) δ 1.05-1.41 (6H, m), 1.34 (9H, s), 1.75-1.85 (3H, m), 2.06 (2H, t), 3.44-3.51 (1H, m), 3.73-3.79 (1H, m), 4.05 (1H, s), 4.22 (2H, q), 7.32-7.34 (2H, m), 7.48-7.52 (3H, m), 7.72 (1H, s)

### Reference Example 24

### Ethyl 1-[(1S,2S)-2-aminocyclohexyl]-5-phenyl-1H-imidazole-4-carboxylate

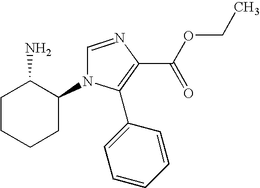

(1S,2S)-Cyclohexane-1,2-diamine (1.37 g) and ethyl 3-bromo-2-isocyano-3-phenylacrylate (1.12 g) were dissolved in DMF (5 ml), and the mixture was stirred at room temperature for 15 hr. Saturated brine was added to the reaction mixture, and the liberated oil was extracted with ethyl acetate. The extract was dried over anhydrous magnesium sulfate, and concentrated under reduced pressure. The residue was subjected to silica gel column chromatography, and the fraction eluted with ethyl acetate-methanol (1:0-9:1) was concentrated under reduced pressure to give the object compound (860 mg) as an oil.

¹H-NMR (CDCl₃) δ 1.02-1.44 (6H, m), 1.21 (3H, t), 1.59-1.81 (3H, m), 1.95-2.00 (2H, m), 3.02 (1H, dt), 3.43 (1H, dt), 4.22 (2H, q), 7.36-7.38 (2H, m), 7.46-7.49 (3H, m), 7.69 (1H, s)

In the same manner as in Reference Example 24, the following compounds (Reference Examples 25-26) were obtained.

### Reference Example 25

### Ethyl 1-[(1R,2R)-2-aminocyclohexyl]-5-phenyl-1H-imidazole-4-carboxylate

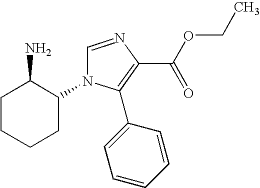

¹H-NMR (CDCl₃) δ 1.03-1.39 (3H, m), 1.22 (3H, t), 1.45 (2H, br s), 1.59-1.82 (3H, t), 1.96-2.01 (2H, m), 3.02 (1H, dt), 3.44 (1H, dt), 4.22 (2H, q), 7.36-7.38 (2H, m), 7.44-7.50 (3H, m), 7.69 (1H, s)

### Reference Example 26

### Ethyl 1-(cis-2-aminocyclohexyl)-5-phenyl-1H-imidazole-4-carboxylate

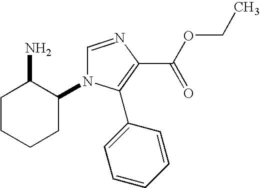

¹H-NMR (CDCl₃) δ 1.19-1.85 (12H, m), 2.17-2.31 (1H, m), 3.03 (1H, br s), 3.84-3.90 (1H, m), 4.22 (2H, q), 7.30-7.33 (2H, m), 7.44-7.48 (3H, m), 7.84 (1H, s)

### Reference Example 27

### 4-(4-Oxocyclohexyl)morpholin-3-one

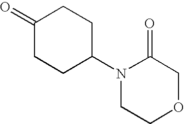

4-(1,4-Dioxaspiro[4.5]dec-8-yl)morpholin-3-one (1.24 g) was dissolved in acetic acid-THF-water (4:2:1, 40 ml), and the solution was stirred at 65° C. for 17 hr. The reaction mixture was concentrated under reduced pressure. The residue was subjected to silica gel column chromatography, and the fraction eluted with ethyl acetate-hexane (1:4) was concentrated under reduced pressure to give the object compound (700 mg) as an oil.

¹H-NMR (CDCl₃) δ 1.67-2.09 (4H, m), 2.43-2.63 (4H, m), 3.30 (2H, t), 3.85-3.92 (2H, m), 4.22 (2H, s), 4.93-5.04 (1H, m)

### Reference Example 28

### 4-(1-Oxaspiro[2.5]oct-6-yl)morpholin-3-one

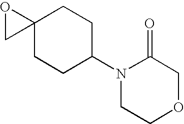

Trimethylsulfoxonium iodide (5.4 g) was dissolved in DMSO (40 ml). Sodium hydride (60% in oil, 972 mg) was added, and the mixture was stirred at room temperature for 30 min. A solution of 4-(4-oxocyclohexyl)morpholin-3-one (4.0 g) in DMSO (80 ml) was added thereto, and the mixture was further stirred at room temperature for 2 hr. The reaction mixture was poured into ice water, and extracted with ethyl acetate-THF (1:1). The extract was dried over anhydrous sodium sulfate, and the solvent was evaporated under reduced pressure. The residue was subjected to silica gel column chromatography, and the fraction eluted with ethyl acetate was concentrated under reduced pressure to give the object compound (2.0 g) as an oil.

¹H-NMR (CDCl₃) δ 1.30-1.38 (2H, m), 1.69-1.89 (4H, m), 2.05-2.16 (2H, m), 2.69 (2H, s), 3.32-3.35 (2H, m), 3.87-3.90 (2H, m), 4.20 (2H, s), 4.59-4.69 (1H, m)

### Reference Example 29

### 4-[4-(Aminomethyl)-4-hydroxycyclohexyl]morpholin-3-one

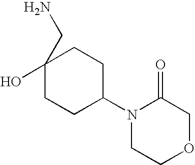

4-(1-Oxaspiro[2.5]oct-6-yl)morpholin-3-one (2.0 g) and benzylamine (3.0 g) were dissolved in ethanol (20 ml), and the solution was stirred at 80° C. for 12 hr. The reaction mixture was concentrated under reduced pressure. The residue was subjected to silica gel column chromatography, and the fraction eluted with ethyl acetate was concentrated under reduced pressure. This was dissolved in methanol (20 ml), 20% palladium hydroxide-carbon (50% containing water, 200 mg) was added thereto, and the mixture was subjected to catalytic reduction at ambient temperature and normal pressure for 12 hr. The catalyst was filtered off, and the filtrate was concentrated under reduced pressure. The residue was suspended in ethyl acetate, and the suspension was washed with saturated aqueous sodium hydrogen carbonate, and dried over anhydrous sodium sulfate. The solvent was evaporated under reduced pressure to give the object compound (1.5 g) as an oil.

¹H-NMR (CDCl₃) δ 1.30-1.46 (2H, m), 1.48-1.59 (2H, m), 1.64-1.73 (2H, m), 1.77-2.02 (4H, m), 2.53-2.63 (2H, m), 2.62 (1H, s), 3.29-3.39 (2H, m), 3.80-3.91 (2H, m), 4.18-4.19 (2H, m), 4.39-4.55 (1H, m)

### Reference Example 30

### Ethyl 1-{[cis-1-hydroxy-4-(3-oxomorpholino)cyclohexyl]methyl}-5-phenyl-1H-imidazole-4-carboxylate

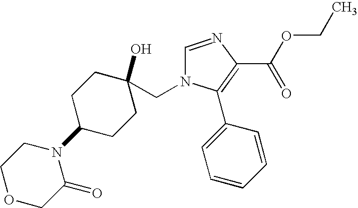

A solution of methyl 3-bromo-2-isocyano-3-phenylacrylate (1.23 g), 4-[4-(aminomethyl)-4-hydroxycyclohexyl]morpholin-3-one (1.5 g) and triethylamine (1.85 ml) in DMF (15 ml) was stirred at room temperature for 12 hr in an argon stream, and poured into water, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate-hexane-methanol (1:1:0-20:0:1) was concentrated under reduced pressure. The crystals were collected by filtration to give the object compound (700 mg).

¹H-NMR (CDCl₃) δ 1.02-2.08 (11H, m), 2.35 (2H, s), 3.20-3.37 (3H, m), 3.75-3.91 (4H, m), 4.07-4.51 (4H, m), 7.09-7.56 (5H, m), 7.88 (1H, s)

### Reference Example 31

### Ethyl 5-(3-fluorophenyl)-1-[(1S,2S)-2-hydroxycyclohexyl]-1H-imidazole-4-carboxylate

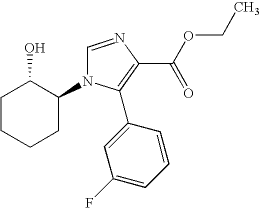

Sodium hydride (60% in oil, 10.1 g) was suspended in THF (200 ml), and the suspension was ice-cooled. A solution of methyl isocyanoacetate (21.8 g) and 3-fluorobenzenecarbaldehyde (23.3 g) in THF (50 ml) was added dropwise thereto. After the completion of the dropwise addition, the mixture was stirred at room temperature for 3 hr. The reaction mixture was ice-cooled, acetic acid (40 ml) was gradually added thereto, and the mixture was poured into ice water, and extracted with ethyl acetate. The organic layer was washed successively with water, saturated aqueous sodium hydrogen carbonate, water and saturated brine, dried over anhydrous magnesium sulfate, and concentrated under reduced pressure. The residue was subjected to silica gel column chromatography, and the fraction eluted with ethyl acetate-hexane (7:3) was concentrated under reduced pressure to give ethyl 3-(3-fluorophenyl)-2-(formylamino)acrylate (34.5 g) as a solid.

The total amount thereof was dissolved in carbon tetrachloride-chloroform (1:1, 400 ml), and the solution was ice-cooled. NBS (27.1 g) was added thereto, and the mixture was stirred at 0° C. for 1.5 hr, and then at room temperature for 3 hr. The reaction mixture was ice-cooled again, triethylamine (21.2 ml) was added, and the mixture was stirred at 0° C. for 20 min, and then at room temperature for 40 min. The reaction mixture was washed successively with water and saturated brine, dried over anhydrous magnesium sulfate, and concentrated under reduced pressure. The residue was subjected to silica gel column chromatography, and the fraction eluted with ethyl acetate-hexane (1:2) was concentrated under reduced pressure to give ethyl 3-bromo-3-(3-fluorophenyl)-2-(formylamino)acrylate (39.2 g) as an oil.

The total amount thereof and triethylamine (45.3 ml) were dissolved in diethyl ether (300 ml), and the solution was ice-cooled. A solution of phosphorus oxychloride (21.0 ml) in diethyl ether (100 ml) was added dropwise, and the mixture was stirred at 0° C. for 3 hr. The reaction mixture was ice-cooled, poured into 10% potassium carbonate aqueous solution (400 ml), and the mixture was vigorously stirred at room temperature for 2 hr. The organic layer was washed successively with water and saturated brine, dried over anhydrous magnesium sulfate, and concentrated under reduced pressure. The residue was subjected to silica gel column chromatography, and the fraction eluted with ethyl acetate-hexane (3:17) was concentrated under reduced pressure to give ethyl 3-bromo-3-(3-fluorophenyl)-2-(isocyano)acrylate (19.76 g) as an oil.

The total amount thereof was dissolved in DMF (50 ml), the solution was added to a solution of (1S,2S)-2-aminocyclohexanol (9.6 g) and triethylamine (21.0 ml) in DMF (150 ml) under ice-cooling, and the mixture was stirred at room temperature for 12 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate was concentrated under reduced pressure to give the object compound (9.15 g) as an amorphous solid.

¹H-NMR (CDCl₃) δ 1.12 (3H, t), 1.20-1.44 (4H, m), 1.55-1.82 (3H, m), 1.84-1.96 (1H, m), 2.04-2.17 (1H, m), 3.49-3.63 (1H, m), 3.79-3.93 (1H, m), 4.06-4.17 (2H, m), 7.07-7.17 (2H, m), 7.35-7.49 (2H, m), 7.63 (1H, s)

MS (ESI+, m/e) 333 (M+1)

In the same manner as in Reference Example 31, the following compounds (Reference Examples 32-38) were obtained.

### Reference Example 32

### Methyl 1-[(1S,2S)-2-aminocyclohexyl]-5-(3-fluorophenyl)-1H-imidazole-4-carboxylate

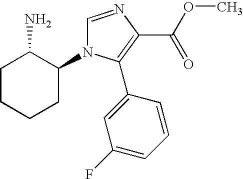

¹H-NMR (CDCl₃) δ 1.07-1.45 (6H, m), 1.60-1.83 (3H, m), 1.93-2.03 (2H, m), 3.05 (1H, dt), 3.43 (1H, dt), 3.78 (3H, s), 7.23-7.27 (2H, m), 7.44-7.50 (1H, m), 7.69 (1H, s)

### Reference Example 33

### Methyl 1,5-dicyclohexyl-1H-imidazole-4-carboxylate

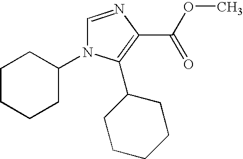

¹H-NMR (CDCl₃) δ 1.15-1.55 (6H, m), 1.56-2.15 (14H, m), 3.25 (1H, br s), 3.88 (3H, s), 3.93-4.05 (1H, m), 7.46 (1H, s)

### Reference Example 34

### Methyl 1-cyclohexyl-5-cyclopropyl-1H-imidazole-4-carboxylate

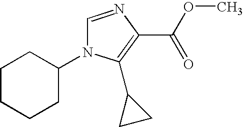

¹H-NMR (CDCl₃) δ 0.74-0.85 (2H, m), 1.06-1.18 (2H, m), 1.19-1.34 (2H, m), 1.37-1.52 (2H, m), 1.55-1.86 (5H, m), 1.95 (2H, s), 2.08 (2H, s), 3.88 (3H, s), 4.28 (1H, tt), 7.48 (1H, s)

### Reference Example 35

### Ethyl 5-(2-fluorophenyl)-1-[(1S,2R)-2-hydroxycyclohexyl]-1H-imidazole-4-carboxylate

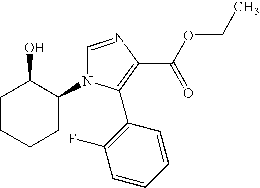

MS (ESI+, m/e) 333 (M+1)

### Reference Example 36

### Ethyl 5-(3,5-difluorophenyl)-1-[(1S,2R)-2-hydroxycyclohexyl]-1H-imidazole-4-carboxylate

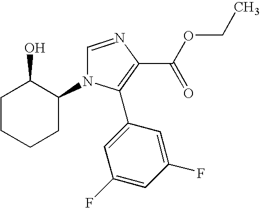

¹H-NMR (CDCl₃) δ 1.10 (3H, t), 1.19-1.77 (6H, m), 1.83-2.02 (2H, m), 2.30 (1H, qd), 3.64-3.76 (1H, m), 4.08-4.20 (3H, m), 6.77-6.85 (2H, m), 6.92 (1H, tt), 7.84 (1H, s)

### Reference Example 37

### Ethyl 5-(2,3-difluorophenyl)-1-[(1S,2S)-2-hydroxycyclohexyl]-1H-imidazole-4-carboxylate

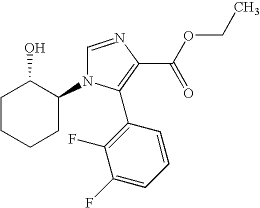

¹H-NMR (CDCl₃) δ 1.13-1.43 (5H, m), 1.53-1.86 (4H, m), 1.98-2.22 (2H, m), 2.65-2.90 (1H, m), 3.51 (1H, dd), 3.75-3.86 (1H, m), 4.12-4.30 (2H, m), 7.15-7.22 (1H, m), 7.24-7.35 (2H, m), 7.72-7.75 (1H, m)

### Reference Example 38

### Ethyl 5-(4-fluorophenyl)-1-[(1S,2S)-2-hydroxycyclohexyl]-1H-imidazole-4-carboxylate

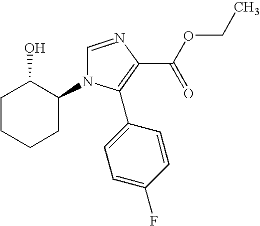

¹H-NMR (CDCl₃) δ 1.14 (3H, t), 1.23-1.37 (2H, m), 1.62-1.69 (1H, m), 1.70-1.83 (2H, m), 1.89-2.03 (1H, m), 2.07-2.17 (1H, m), 3.52-3.66 (1H, m), 3.79-3.93 (1H, m), 4.15 (2H, q), 7.11-7.27 (3H, m), 7.33-7.46 (1H, m), 7.66 (1H, s)

### Reference Example 39

### Ethyl 1-{(1S,2S)-2-[(ethoxycarbonyl)amino]cyclohexyl}-5-phenyl-1H-imidazole-4-carboxylate

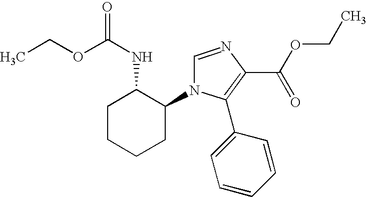

Ethyl 1-[(1S,2S)-2-aminocyclohexyl]-5-phenyl-1H-imidazole-4-carboxylate (850 mg) and triethylamine (378 mg) were dissolved in dichloromethane (10 ml), and the solution was ice-cooled. Ethyl chloroformate (322 mg) was added, and the mixture was stirred at 0° C. for 2 hr. The mixture was concentrated under reduced pressure, and to the residue was added ethyl acetate. The mixture was washed successively with water and saturated brine, dried over anhydrous magnesium sulfate, and concentrated under reduced pressure. The residue was subjected to silica gel column chromatography, and the fraction eluted with ethyl acetate-hexane (3:7-7:3) was concentrated under reduced pressure to give the object compound (450 mg).

¹H-NMR (CDCl₃) δ 1.05-1.23 (8H, m), 1.32-1.45 (1H, m), 1.79 (3H, t), 2.05-2.08 (2H, m), 3.52 (1H, br t), 3.85 (1H, br s), 3.79-4.05 (2H, m), 4.15-4.25 (3H, m), 7.30-7.32 (2H, m), 7.48-7.51 (3H, m), 7.72 (1H, s)

In the same manner as in Reference Example 39, the following compounds (Reference Examples 40-44) were obtained.

### Reference Example 40

### Ethyl 1-{(1S,2S)-2-[(methoxycarbonyl)amino]cyclohexyl}-5-phenyl-1H-imidazole-4-carboxylate

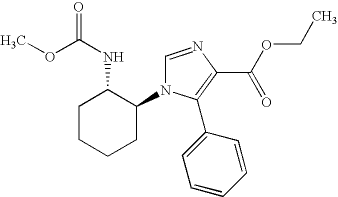

¹H-NMR (CDCl₃) δ 1.17-1.23 (5H, m), 1.32-1.45 (1H, m), 1.74-1.83 (3H, m), 2.05-2.07 (2H, m), 3.55 (4H, s), 3.85 (1H, br s), 4.15-4.24 (2H, m), 4.42 (1H, br s), 7.11-7.48 (5H, m), 7.72 (1H, s)

### Reference Example 41

### Ethyl 1-{(1R,2R)-2-[(ethoxycarbonyl)amino]cyclohexyl}-5-phenyl-1H-imidazole-4-carboxylate

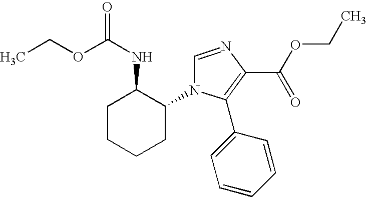

¹H-NMR (CDCl₃) δ 1.11-1.26 (8H, m), 1.31-1.46 (1H, m), 1.74-1.82 (3H, m), 2.05-2.08 (2H, m), 3.53 (1H, t), 3.86 (1H, br s), 3.97-4.05 (2H, m), 4.15-4.25 (3H, m), 7.30-7.32 (2H, m), 7.47-7.49 (3H, m), 7.72 (1H, s)

### Reference Example 42

### Ethyl 1-{cis-2-[(ethoxycarbonyl)amino]cyclohexyl}-5-phenyl-1H-imidazole-4-carboxylate

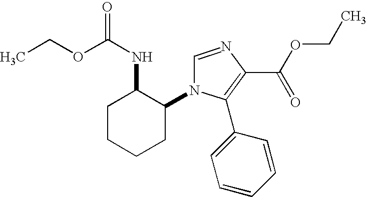

¹H-NMR (CDCl₃) δ 1.14-1.44 (9H, m), 1.57-1.60 (1H, m), 1.71-1.74 (1H, m), 1.87-1.91 (3H, m), 3.89-4.02 (4H, m), 4.14-4.22 (2H, m), 4.93 (1H, br d), 7.43-7.47 (5H, m), 7.57 (1H, s)

### Reference Example 43

### Methyl 5-(3-fluorophenyl)-1-{(1S,2S)-2-[(methoxycarbonyl)amino]cyclohexyl}-1H-imidazole-4-carboxylate

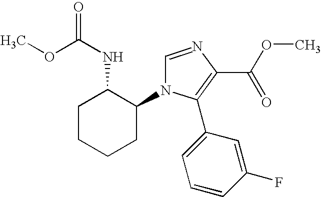

¹H-NMR (CDCl₃) δ 1.15-1.46 (4H, m), 1.77-1.85 (3H, m), 2.05-2.06 (2H, m), 3.55 (3H, br s), 3.75 (3H, s), 3.84 (1H, br s), 7.02-7.10 (2H, m), 7.19 (1H, dt), 7.43-7.51 (1H, m), 7.72 (1H, s)

### Reference Example 44

### Methyl 1-{(1S,2S)-2-[(ethoxycarbonyl)amino]cyclohexyl}-5-(3-fluorophenyl)-1H-imidazole-4-carboxylate

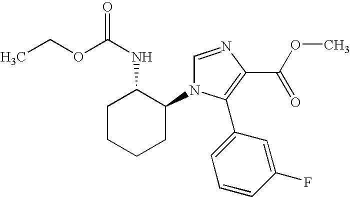

¹H-NMR (CDCl₃) δ 1.15-1.27 (5H, m), 1.33-1.46 (1H, m), 1.71-1.85 (3H, m), 2.05-2.09 (2H, m), 3.56 (1H, dt), 3.74 (3H, s), 3.85 (1H, br s), 3.96-4.04 (2H, m), 4.47 (1H, br d), 7.03-7.11 (2H, m), 7.15-7.21 (1H, m), 7.43-7.50 (1H, m), 7.74 (1H, s)

### Reference Example 45

### Ethyl 1-{(1S,2S)-2-[(methylsulfonyl)oxy]cyclohexyl}-5-phenyl-1H-imidazole-4-carboxylate

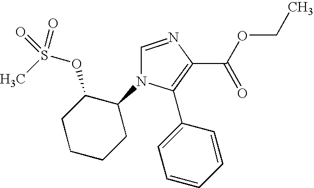

Ethyl 1-[(1S,2S)-2-hydroxycyclohexyl]-5-phenyl-1H-imidazole-4-carboxylate (3.14 g) was dissolved in pyridine (50 ml), and the solution was ice-cooled. Methanesulfonyl chloride (1.49 g) was added dropwise over 1 min, and the mixture was stirred at 0° C. for 1 hr. The reaction mixture was concentrated under reduced pressure, and the residue was dissolved in ethyl acetate (50 ml). The insoluble material was filtered off, and the filtrate was concentrated under reduced pressure. The residue was subjected to silica gel column chromatography, and the fraction eluted with ethyl acetate-hexane (1:1-1:0) was concentrated under reduced pressure to give the object compound (3.35 g) as an amorphous solid.

¹H-NMR (CDCl₃) δ 1.20 (3H, t), 1.35-1.56 (2H, m), 1.77-1.87 (3H, m), 2.09-2.14 (1H, m), 2.34-2.40 (1H, m), 2.61 (3H, s), 3.80-3.89 (1H, m), 4.17-4.26 (2H, m), 4.74-4.82 (1H, m), 7.33-7.35 (2H, m), 7.49-7.52 (3H, m), 7.77 (1H, s)

### Reference Example 46

### Ethyl 1-[(1S,2R)-2-azidocyclohexyl]-5-phenyl-1H-imidazole-4-carboxylate

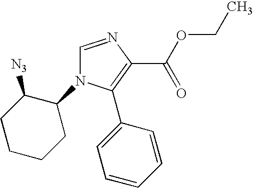

A solution of ethyl 1-{(1S,2S)-2-[(methylsulfonyl)oxy]cyclohexyl}-5-phenyl-1H-imidazole-4-carboxylate (3.0 g) and sodium azide (3.9 g) in DMSO (30 ml) was stirred at 80° C. for 15 hr. The reaction mixture was poured into ice water, and the liberated oil was extracted with ethyl acetate. The extract was washed with saturated brine, dried over anhydrous magnesium sulfate, and concentrated under reduced pressure. The residue was subjected to silica gel column chromatography, and the fraction eluted with ethyl acetate-hexane (3:7-7:3) was concentrated under reduced pressure to give the object compound (2.1 g).

¹H-NMR (CDCl₃) δ 1.22 (3H, t), 1.28-1.44 (2H, m), 1.50-1.60 (2H, m), 1.78-2.03 (3H, m), 2.10-2.24 (1H, m), 3.69-3.70 (1H, m), 3.83-3.89 (1H, m), 4.22 (2H, q), 7.30-7.33 (2H, m), 7.46-7.51 (3H, m), 7.79 (1H, s)

### Reference Example 47

### Ethyl 1-[(1S,2R)-2-fluorocyclohexyl]-5-phenyl-1H-imidazole-4-carboxylate

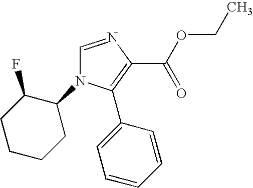

A solution of ethyl 1-{(1S,2S)-2-[(methylsulfonyl)oxy]cyclohexyl}-5-phenyl-1H-imidazole-4-carboxylate (785 mg) and TBAF (1.40 g) in acetonitrile (10 ml) was heated under reflux for 20 hr, and concentrated under reduced pressure. Ethyl acetate was added to the residue, and the mixture was washed successively with water and saturated brine, dried over anhydrous magnesium sulfate, and concentrated under reduced pressure. The residue was subjected to silica gel column chromatography, and the fraction eluted with ethyl acetate-hexane (3:7-7:3) was concentrated under reduced pressure to give the object compound (430 mg).

¹H-NMR (CDCl₃) δ 1.22 (3H, t), 1.25-1.34 (1H, m), 1.42-1.67 (3H, m), 1.81-1.91 (2H, m), 2.04-2.24 (2H, m), 3.71-3.86 (1H, m), 4.23 (2H, q), 4.70 (1H, d), 7.28-7.32 (2H, m), 7.47-7.49 (3H, m), 7.82 (1H, d)

### Reference Example 48

### Ethyl 1-[(1S,2R)-2-aminocyclohexyl]-5-phenyl-1H-imidazole-4-carboxylate

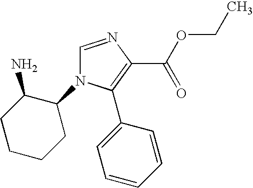

Ethyl 1-[(1S,2R)-2-azidocyclohexyl]-5-phenyl-1H-imidazole-4-carboxylate (2.00 g) was dissolved in methanol (15 ml), 10% palladium-carbon (50% containing water, 500 mg) was added thereto, and the mixture was subjected to catalytic reduction at ambient temperature and normal pressure for 5 hr. The catalyst was filtered off, and the filtrate was concentrated under reduced pressure to give the object compound (1.84 g).

¹H-NMR (CDCl₃) δ 0.98 (2H, br s), 1.20-1.88 (10H, m), 2.18-2.31 (1H, m), 3.03 (1H, br s), 3.84-3.90 (1H, m), 4.22 (2H, q), 7.30-7.33 (2H, m), 7.44-7.51 (3H, m), 7.84 (1H, s)

### Reference Example 49

### Ethyl 1-((1S,2R)-2-{[(benzyloxy)carbonyl]amino}cyclohexyl)-5-phenyl-1H-imidazole-4-carboxylate

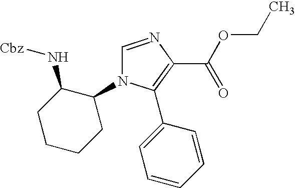

Ethyl 1-[(1S,2R)-2-aminocyclohexyl]-5-phenyl-1H-imidazole-4-carboxylate (1.80 g) was dissolved in THF (10 ml), and the solution was ice-cooled. Triethylamine (865 mg) and benzyl chloroformate (1.18 g) were added. The mixture was stirred at 0° C. for 1 hr, and concentrated under reduced pressure. Ethyl acetate was added to the residue, and the mixture was washed successively with 6% aqueous sodium bicarbonate and saturated brine, dried over anhydrous magnesium sulfate, and concentrated under reduced pressure. The residue was subjected to basic silica gel column chromatography, and the fraction eluted with ethyl acetate-hexane (1:1-1:0) was concentrated under reduced pressure to give the object compound (1.61 g).

¹H-NMR (CDCl₃) δ 1.18 (3H, t), 1.23-1.44 (3H, m), 1.56-1.60 (1H, m), 1.68-1.75 (1H, m), 1.87-1.90 (3H, m), 3.91-4.04 (1H, m), 4.20 (2H, q), 4.92-5.07 (3H, m), 7.34-7.47 (10H, m), 7.58 (1H, s)

### Reference Example 50

### Ethyl 1-(2-oxocyclohexyl)-5-phenyl-1H-imidazole-4-carboxylate

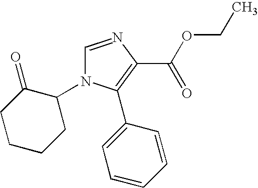

Ethyl 1-[(1S,2S)-2-hydroxycyclohexyl]-5-phenyl-1H-imidazole-4-carboxylate (5.5 g) and triethylamine (5.3 g) were dissolved in DMSO (55 ml), and the solution was cooled to 15° C.

A solution of pyridine-sulfur trioxide complex (8.4 g) in DMSO (20 ml) was added dropwise over 5 min. The mixture was stirred at room temperature for 2 hr. The reaction mixture was poured into ice water, and the liberated oil was extracted with ethyl acetate. The extract was washed successively with 6% aqueous sodium bicarbonate, 10% aqueous citric acid solution and saturated brine, dried over anhydrous magnesium sulfate, and concentrated under reduced pressure. The residue was subjected to silica gel column chromatography, and the fraction eluted with ethyl acetate-hexane (1:1-1:0) was concentrated under reduced pressure to give the object compound (5.4 g).

¹H-NMR (CDCl₃) δ 1.21 (3H, t), 1.70-1.76 (2H, m), 2.03-2.30 (4H, m), 2.39-2.45 (1H, m), 2.54-2.60 (1H, m), 4.22 (2H, q), 4.46 (1H, dd), 7.24-7.27 (2H, m), 7.42-7.46 (3H, m), 7.58 (1H, s)

In the same manner as in Reference Example 50, the following compound (Reference Example 51) was obtained.

### Reference Example 51

### Ethyl 5-(3-fluorophenyl)-1-(2-oxocyclohexyl)-1H-imidazole-4-carboxylate

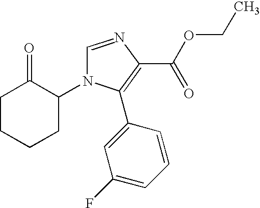

MS (ESI+, m/e) 331 (M+1)

### Reference Example 52

### Ethyl 1-[(3R,4S)-1-oxaspiro[2.5]oct-4-yl]-5-phenyl-1H-imidazole-4-carboxylate and ethyl 1-[(3S,4R)-1-oxaspiro[2.5]oct-4-yl]-5-phenyl-1H-imidazole-4-carboxylate

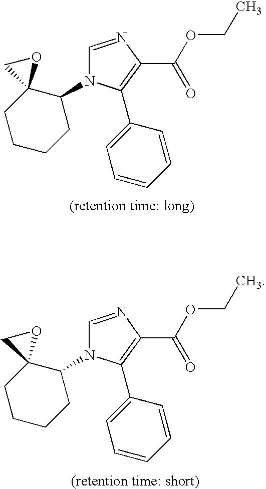

Trimethylsulfoxonium iodide (17.96 g) was dissolved in DMSO (300 ml), and sodium hydride (60% in oil, 3.26 g) was added at room temperature. After stirring for 30 min, the mixture was cooled to 15 to 20° C. A solution of ethyl 1-(2-oxocyclohexyl)-5-phenyl-1H-imidazole-4-carboxylate (21.24 g) in DMSO (75 ml) was added dropwise thereto over 20 min, and the mixture was stirred at room temperature for 30 min. The reaction mixture was poured into ice water, and the liberated oil was extracted with ethyl acetate. The extract was washed with saturated brine, dried over anhydrous magnesium sulfate, and concentrated under reduced pressure. The residue was subjected to basic silica gel column chromatography, and the fraction eluted with ethyl acetate-hexane (3:7-7:3) was concentrated under reduced pressure to give a racemic mixture (18.54 g) of ethyl 1-[(3R,4S)-1-oxaspiro[2.5]oct-4-yl]-5-phenyl-1H-imidazole-4-carboxylate and ethyl 1-[(3S,4R)-1-oxaspiro[2.5]oct-4-yl]-5-phenyl-1H-imidazole-4-carboxylate.

¹H-NMR (CDCl₃) δ 1.23 (3H, t), 1.35-1.44 (2H, m), 1.65-2.17 (8H, m), 1.51 (1H, d), 4.11 (1H, dd), 4.22 (2H, q), 7.26-7.30 (2H, m), 7.46-7.50 (3H, m), 7.71 (1H, s)

The obtained racemate was optically resolved by normal phase chiral HPLC under the following conditions.

column: CHIRALPAK AD 50 mm ID×500 mL
mobile phase: hexane-ethanol (9:1)
flow rate: 80 ml/min
temperature: 30° C.
detection: UV (254 nm)
injection volume-concentration: 10 mg/ml, 47 ml (load: 470 mg)

In the same manner as in Reference Example 52, the following compound (Reference Example 53) was obtained.

### Reference Example 53

### Ethyl 5-(3-fluorophenyl)-1-[(3R,4S)-1-oxaspiro[2.5]oct-4-yl]-1H-imidazole-4-carboxylate and ethyl 5-(3-fluorophenyl)-1-[(3S,4R)-1-oxaspiro[2.5]oct-4-yl]-1H-imidazole-4-carboxylate

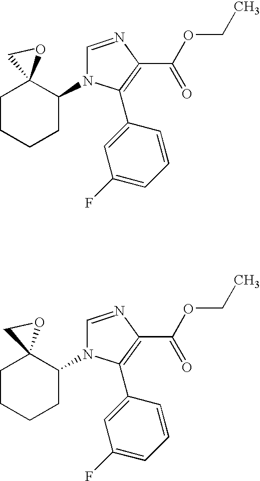

MS (ESI+, m/e) 344 (M+1)

MS (ESI+, m/e) 344 (M+1)

### Reference Example 54

### Ethyl 1-{2-[(benzylamino)methyl]-2-hydroxycyclohexyl}-5-phenyl-1H-imidazole-4-carboxylate

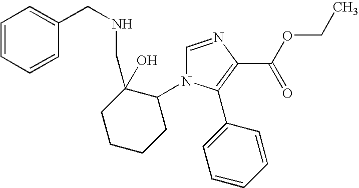

Ethyl 1-(1-oxaspiro[2.5]oct-4-yl)-5-phenyl-1H-imidazole-4-carboxylate (680 mg) and benzylamine (430 mg) were dissolved in acetonitrile (10 ml). Lithium perchlorate (426 mg) was added, and the mixture was heated under reflux for 15 hr. The reaction mixture was concentrated under reduced pressure, and to the residue was added ethyl acetate. The mixture was washed successively with water and saturated brine, dried over anhydrous magnesium sulfate, and concentrated under reduced pressure. The residue was subjected to silica gel column chromatography, and the fraction eluted with ethyl acetate-hexane (1:1-7:3) was concentrated under reduced pressure to give the object compound (785 mg) as an amorphous solid.

¹H-NMR (CDCl₃) δ 1.01 (1H, dt), 1.15-1.25 (1H, m), 1.21 (3H, t), 1.48-1.52 (1H, m), 1.65-1.86 (5H, m), 2.11 (2H, s), 2.26 (2H, dq), 3.52 (1H, dd), 3.67 (2H, s), 4.21 (2H, dq), 7.10-7.18 (4H, m), 7.28-7.46 (6H, m), 7.06 (1H, s)

### Reference Example 55

### Ethyl 1-(2-{[(ethoxycarbonyl)amino]methyl}-2-hydroxycyclohexyl)-5-phenyl-1H-imidazole-4-carboxylate

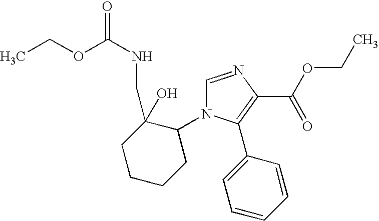

Ethyl 1-{2-[(benzylamino)methyl]-2-hydroxycyclohexyl}-5-phenyl-1H-imidazole-4-carboxylate (770 mg) was dissolved in methanol (8 ml), 20% palladium hydroxide-carbon (50% containing water, 200 mg) was added thereto, and the mixture was subjected to catalytic reduction at ambient temperature and normal pressure for 12 hr. The catalyst was filtered off, and the filtrate was concentrated under reduced pressure to give ethyl 1-[2-(aminomethyl)-2-hydroxycyclohexyl]-5-phenyl-1H-imidazole-4-carboxylate (590 mg).

¹H-NMR (CDCl₃) δ 1.02 (1H, dt), 1.17 (3H, t), 1.22-1.28 (1H, m), 1.51-1.54 (1H, m), 1.66-1.88 (4H, m), 2.20-2.33 (3H, m), 2.56 (3H, br s), 3.58 (1H, dd), 4.13-4.24 (2H, m), 7.29 (2H, br s), 7.45-7.49 (3H, m), 8.08 (1H, s)

The above-mentioned ethyl 1-[2-(aminomethyl)-2-hydroxycyclohexyl]-5-phenyl-1H-imidazole-4-carboxylate (584 mg) and triethylamine (258 mg) were dissolved in dichloromethane (10 ml), and the solution was ice-cooled. Ethyl chloroformate (203 mg) was added, and the mixture was stirred at 0° C. for 2 hr, and concentrated under reduced pressure. To the residue was added ethyl acetate. The mixture was washed successively with water and saturated brine, dried over anhydrous magnesium sulfate, and concentrated under reduced pressure. The residue was subjected to silica gel column chromatography, and the fraction eluted with ethyl acetate-hexane (3:7-7:3) was concentrated under reduced pressure to give the object compound (615 mg) as an amorphous solid.

¹H-NMR (CDCl₃) δ 1.07-1.22 (8H, m), 1.51-1.83 (6H, m), 2.22 (1H, dq), 2.73-2.84 (2H, m), 3.63 (1H, dd), 3.91 (1H, br s), 4.05 (2H, dq), 4.20 (2H, q), 5.47 (1H, br t), 7.30 (2H, br s), 7.48-7.51 (3H, m), 8.05 (1H, s)

### Reference Example 56

### Ethyl 1-((1S,2R)-2-hydroxy-2-{[3-(methylthio)propoxy]methyl}cyclohexyl)-5-phenyl-1H-imidazole-4-carboxylate

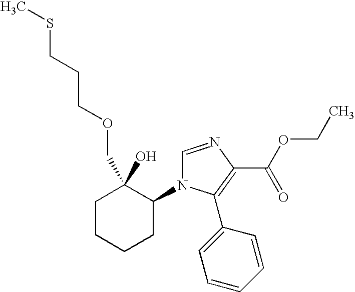

Sodium hydride (60% in oil, 88 mg) was suspended in DMF (3 ml), 3-(methylthio)propan-1-ol (267 μl) was added thereto, and the mixture was stirred at room temperature for 30 min. To this was added ethyl 1-[(3R,4S)-1-oxaspiro[2.5]oct-4-yl]-5-phenyl-1H-imidazole-4-carboxylate (240 mg), and the mixture was stirred at 60° C. for 15 hr. The reaction mixture was neutralized with 1N hydrochloric acid, and extracted with ethyl acetate. The extract was washed with saturated brine, dried over anhydrous magnesium sulfate, and concentrated under reduced pressure to give the object compound (318 mg).

MS (ESI+, m/e) 433 (M+1)

### Reference Example 57

### Ethyl 1-(2-methylenecyclohexyl)-5-phenyl-1H-imidazole-4-carboxylate

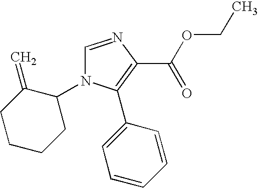

Ethyl 1-(2-oxocyclohexyl)-5-phenyl-1H-imidazole-4-carboxylate (6.5 g) and methyltriphenylphosphonium bromide (11.15 g) were dissolved in THF (100 ml), and potassium tert-butoxide (3.5 g) was added at 15 to 17° C. After stirring at room temperature for 2 hr, the insoluble material was filtered off, and the filtrate was concentrated under reduced pressure. To the residue was added ethyl acetate. The mixture was washed successively with water and saturated brine, dried over anhydrous magnesium sulfate, and concentrated under reduced pressure. The residue was subjected to basic silica gel column chromatography, and the fraction eluted with ethyl acetate-hexane (1:4-3:2) was concentrated under reduced pressure to give the object compound (6.0 g) as an amorphous solid.

¹H-NMR (CDCl₃) δ 1.24 (3H, t), 1.37-1.46 (2H, m), 1.81-2.01 (4H, m), 2.09-2.13 (1H, m), 2.46 (1H, d), 4.19-4.28 (4H, m), 4.85 (1H, s), 7.34-7.36 (2H, m), 7.43-7.45 (3H, m), 7.66 (1H, s)

### Reference Example 58

### Ethyl 1-(2-ethoxy-2-{[(ethoxycarbonyl)amino]methyl}cyclohexyl)-5-phenyl-1H-imidazole-4-carboxylate

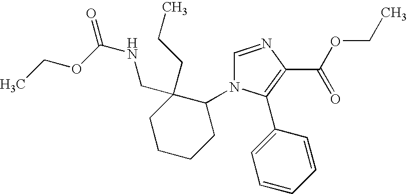

A mixture of ethyl 1-(2-methylenecyclohexyl)-5-phenyl-1H-imidazole-4-carboxylate (4.66 g), ethyl {[(4-nitrophenyl)sulfonyl]oxy}carbamate (8.7 g), calcium oxide (1.68 g) and dichloromethane (100 ml) was stirred at room temperature for 15 hr. The insoluble material was filtered off, and the filtrate was concentrated under reduced pressure. The residue was subjected to basic silica gel column chromatography, and the fraction eluted with ethyl acetate-hexane (1:4-3:2) was concentrated under reduced pressure to give a mixture (ratio 3:2, 2.16 g) as an amorphous solid of ethyl 4-[4-(ethoxycarbonyl)-5-phenyl-1H-imidazol-1-yl]-1-azaspiro[2.5]octane-1-carboxylate and the starting material. This was dissolved in ethanol (15 ml), and boron trifluoride diethyl etherate (425 mg) was added. The mixture was stirred at 70° C. for 26 hr, and concentrated under reduced pressure. Ethyl acetate was added to the residue, and the mixture was washed successively with saturated aqueous sodium hydrogen carbonate and saturated brine, dried over anhydrous magnesium sulfate, and concentrated under reduced pressure. The residue was subjected to basic silica gel column chromatography, and the fraction eluted with ethyl acetate-hexane (3:7-7:3) was concentrated under reduced pressure to give the object compound (276 mg) as an amorphous solid.

¹H-NMR (CDCl₃) δ 0.94 (3H, t), 1.24 (6H, t), 1.44-1.53 (2H, m), 1.53-1.67 (2H, m), 1.80-1.84 (2H, m), 2.00-2.11 (2H, m), 2.64-2.74 (1H, m), 3.00-3.17 (2H, m), 3.64-3.71 (1H, m), 4.05-4.16 (2H, m), 4.18-4.28 (3H, m), 4.55 (1H, br s), 7.34-7.48 (5H, m), 7.67 (1H, s)

### Reference Example 59

### Ethyl 1-cyclohexyl-2-oxo-5-phenyl-2,3-dihydro-1H-imidazole-4-carboxylate

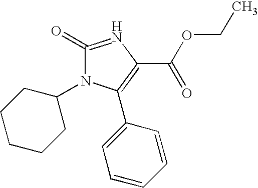

A mixture of cyclohexylurea (755 mg), ethyl 2-diazo-3-oxo-3-phenylpropionate (1.00 g), rhodium (II) acetate dimmer (30 mg), toluene (10 ml) and 1,2-dichloroethane (10 ml) was stirred at 80° C. for 1 hr, and cooled to room temperature. TFA (1.0 ml) was added, and the reaction mixture was stirred at room temperature for 2 days, and concentrated under reduced pressure. The residue was subjected to silica gel column chromatography, and the fraction eluted with ethyl acetate-hexane (1:4-4:1) was concentrated under reduced pressure. The crystals were collected by filtration to give the object compound (1.01 g).

¹H-NMR (CDCl₃) δ 1.10 (6H, t), 1.71 (3H, t), 1.69 (2H, br s), 2.27 (2H, d), 3.44-3.57 (1H, m), 4.10 (2H, q), 7.26-7.37 (2H, m), 7.47 (2H, d), 7.42-7.51 (1H, m), 8.87 (1H, br s)

MS (ESI+, m/e) 315 (M+1)

### Reference Example 60

### Ethyl 1-cyclohexyl-2-ethoxy-5-phenyl-1H-imidazole-4-carboxylate

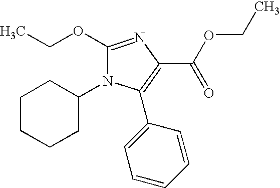

Ethyl 1-cyclohexyl-2-oxo-5-phenyl-2,3-dihydro-1H-imidazole-4-carboxylate (500 mg) was dissolved in dichloromethane (10 ml), and the solution was ice-cooled. Triethyloxonium tetrafluoroborate (1M dichloromethane solution, 5.0 ml) was added dropwise, and the reaction mixture was stirred at room temperature for 14 hr. The reaction mixture was poured into saturated aqueous sodium hydrogen carbonate, and the mixture was extracted with ethyl acetate. The extract was washed successively with water and saturated brine, and dried over anhydrous magnesium sulfate, and the solvent was evaporated under reduced pressure. The residue was subjected to silica gel column chromatography, and the fraction eluted with ethyl acetate-hexane (1:9-4:1) was concentrated under reduced pressure to give the object compound (319 mg).

¹H-NMR (CDCl₃) δ 1.11 (6H, q), 1.45 (3H, t), 1.58 (1H, d), 1.74 (2H, d), 1.65-1.80 (2H, m), 2.05 (1H, dd), 1.97-2.12 (1H, m), 3.51 (1H, t), 4.15 (2H, q), 4.56 (2H, q), 7.26-7.33 (1H, m), 7.29 (1H, dd), 7.40-7.46 (1H, m), 7.43 (2H, d)

MS (ESI+, m/e) 343 (M+1)

### Reference Example 61

### Ethyl 2-chloro-1-cyclohexyl-5-phenyl-1H-imidazole-4-carboxylate

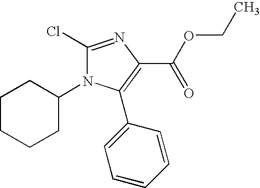

A mixture of ethyl 1-cyclohexyl-2-oxo-5-phenyl-2,3-dihydro-1H-imidazole-4-carboxylate (10.0 g) and phosphoryl chloride (70 ml) was stirred at 100° C. for 31 hr, and cooled to room temperature. The reaction mixture was concentrated under reduced pressure, and the residue was dissolved in ethyl acetate. The solution was washed successively with saturated aqueous sodium hydrogen carbonate and saturated brine, and dried over anhydrous magnesium sulfate, and the solvent was evaporated under reduced pressure. The residue was subjected to silica gel column chromatography, and the fraction eluted with ethyl acetate-hexane (1:9-7:3) was concentrated under reduced pressure to give the object compound (4.69 g).

¹H-NMR (CDCl₃) δ 1.08-1.21 (6H, m), 1.53-1.68 (2H, m), 1.71-1.85 (5H, m), 3.85 (1H, br s), 4.09-4.23 (2H, m), 7.25-7.34 (2H, m), 7.42-7.51 (3H, m)

MS (ESI+, m/e) 333 (M+1)

### Reference Example 62

### Ethyl 3-[(trans-2-hydroxycyclohexyl)amino]-2-nitro-3-phenylacrylate

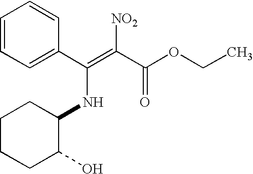

A mixture of ethyl 3-iodo-2-nitro-3-phenylacrylate (7.00 g), trans-2-aminocyclohexanol hydrochloride (4.59 g), triethylamine (12.5 ml) and acetonitrile (60 ml) was stirred at room temperature for 12 hr, and poured into saturated aqueous sodium hydrogen carbonate, and the mixture was extracted with ethyl acetate. The extract was washed with saturated brine, and dried over anhydrous magnesium sulfate, and the solvent was evaporated under reduced pressure. The residue was subjected to silica gel column chromatography, and the fraction eluted with ethyl acetate-hexane (1:4-4:1) was concentrated under reduced pressure to give the object compound (5.28 g).

¹H-NMR (CDCl₃) δ 0.88-1.01 (3H, m), 1.23-1.30 (2H, m), 1.38-1.48 (1H, m), 1.57-1.71 (4H, m), 1.78-1.89 (1H, m), 1.94-2.06 (1H, m), 2.18 (1H, br s), 2.94-3.07 (1H, m), 3.50-3.62 (1H, m), 3.90 (2H, br s), 7.24-7.35 (2H, m), 7.40-7.51 (3H, m)

MS (ESI+, m/e) 335 (M+1)

### Reference Example 63

### Ethyl 1-(trans-2-hydroxycyclohexyl)-2-methyl-5-phenyl-1H-imidazole-4-carboxylate

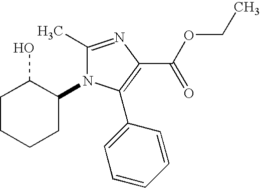

A mixture of ethyl 3-[(trans-2-hydroxycyclohexyl)amino]-2-nitro-3-phenylacrylate (4.49 g), 10% palladium-carbon (50% containing water, 500 mg) and trimethyl orthoacetate (150 ml) was subjected to catalytic reduction at 80° C. for 11 hr under hydrogen pressure (5 kgf/cm²). The catalyst was filtered off, and the filtrate was concentrated under reduced pressure. The residue was subjected to silica gel column chromatography, and the fraction eluted with ethyl acetate-methanol (1:0-9:1) was concentrated under reduced pressure to give the object compound (360 mg).

¹H-NMR (CDCl₃) δ 0.83-0.97 (1H, m), 1.12-1.26 (4H, m), 1.63-1.73 (2H, m), 1.76-1.83 (1H, m), 1.97-2.08 (2H, m), 2.13-2.29 (3H, m), 2.45 (1H, br s), 3.06-3.21 (1H, m), 3.61-3.77 (1H, m), 4.05-4.21 (3H, m), 7.24-7.36 (4H, m), 7.44 (1H, br s)

MS (ESI+, m/e) 329 (M+1)

### Reference Example 64

### 1-[(1S,2S)-2-(Benzyloxy)cyclohexyl]-5-phenyl-1H-imidazole-4-carboxylic acid

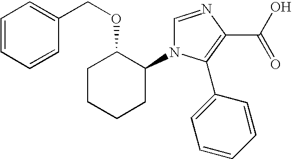

Methyl 1-[(1S,2S)-2-(benzyloxy)cyclohexyl]-5-phenyl-1H-imidazole-4-carboxylate (1.25 g) was dissolved in methanol-THF (1:1, 20 ml). 8N aqueous sodium hydroxide solution (5 ml) was added, and the mixture was stirred at room temperature for 15 hr. The reaction mixture was concentrated under reduced pressure, and partitioned between ethyl acetate and 10% aqueous citric acid solution. The organic layer was washed with saturated brine, and dried over anhydrous sodium sulfate, and the solvent was evaporated under reduced pressure to give the object compound (1.11 g) as an amorphous solid.

¹H-NMR (CDCl₃) δ 1.14-1.29 (3H, m), 1.59-1.87 (3H, m), 1.92-2.06 (1H, m), 2.20-2.36 (1H, m), 3.55 (1H, td), 3.75-3.87 (1H, m), 4.23 (1H, d), 4.48 (1H, d), 7.01 (2H, s), 7.19-7.32 (4H, m), 7.39-7.47 (5H, m), 7.91 (1H, br s)

In the same manner as in Reference Example 64, the following compound (Reference Example 65) was obtained.

### Reference Example 65

### 1-[(1R,2R)-2-(Benzyloxy)cyclohexyl]-5-phenyl-1H-imidazole-4-carboxylic acid

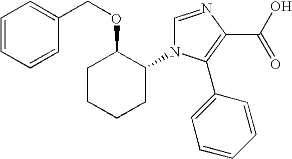

¹H-NMR (CDCl₃) δ 1.14-1.29 (3H, m), 1.59-1.87 (3H, m), 1.92-2.06 (1H, m), 2.20-2.36 (1H, m), 3.55 (1H, td), 3.75-3.87 (1H, m), 4.23 (1H, d), 4.48 (1H, d), 7.01 (2H, s), 7.19-7.32 (4H, m), 7.39-7.47 (5H, m), 7.91 (1H, br s)

### Reference Example 66

### 1-[(1R,2R)-2-(Benzyloxy)cyclopentyl]-5-phenyl-1H-imidazole-4-carboxylic acid

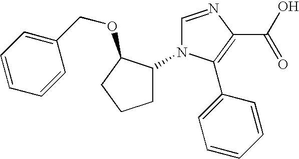

A mixture of methyl 1-[(1R,2R)-2-(benzyloxy)cyclopentyl]-5-phenyl-1H-imidazole-4-carboxylate (680 mg), lithium hydroxide monohydrate (400 mg), THF (4 ml), methanol (4 ml) and water (6 ml) was stirred at 70° C. for 12 hr, and concentrated under reduced pressure. The residual aqueous solution was acidified with 1N hydrochloric acid, and the mixture was extracted with ethyl acetate. The extract was washed with saturated brine, and dried over anhydrous magnesium sulfate, and the solvent was evaporated under reduced pressure to give the object compound (468 mg).

MS (ESI+, m/e) 363 (M+1)

In the same manner as in Reference Example 66, the following compounds (Reference Examples 67-70) were obtained.

### Reference Example 67

### 1-[(2R)-Bicyclo[2.2.1]hept-2-yl]-5-phenyl-1H-imidazole-4-carboxylic acid

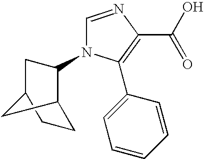

MS (ESI+, m/e) 283 (M+1)

### Reference Example 68

### 1-[Bicyclo[2.2.1]hept-2-yl]-5-phenyl-1H-imidazole-4-carboxylic acid

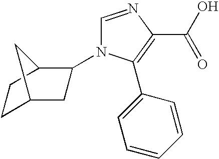

MS (ESI+, m/e) 283 (M+1)

### Reference Example 69

### 1-Cyclohexyl-2-ethoxy-5-phenyl-1H-imidazole-4-carboxylic acid

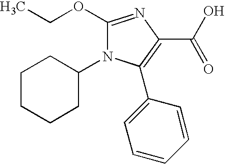

¹H-NMR (CDCl₃) δ 1.12 (3H, br s), 1.46 (3H, t), 1.61 (1H, br s), 1.67-1.83 (2H, m), 1.76 (2H, d), 2.05 (1H, s), 2.07 (1H, d), 3.58 (1H, dd), 4.50 (2H, q), 7.25-7.37 (2H, m), 7.38-7.49 (1H, m), 7.44 (2H, d)

MS (ESI+, m/e) 315 (M+1)

### Reference Example 70

### 2-Chloro-1-cyclohexyl-5-phenyl-1H-imidazole-4-carboxylic acid

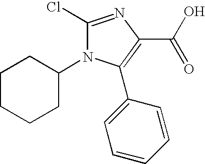

¹H-NMR (DMSO-d₆) δ 0.96-1.11 (3H, m), 1.53 (1H, br s), 1.68-1.83 (4H, m), 1.87-2.03 (2H, m), 3.62-3.77 (1H, m), 7.34-7.42 (2H, m), 7.44-7.53 (3H, m)

MS (ESI+, m/e) 305 (M+1)

### Reference Example 71

### 1-[(1S,2R)-2-Azidocyclohexyl]-5-phenyl-1H-imidazole-4-carboxylic acid

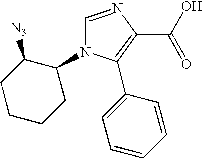

A solution of ethyl 1-[(1S,2R)-2-azidocyclohexyl]-5-phenyl-1H-imidazole-4-carboxylate (2.30 g) and potassium hydroxide (1.15 g) in ethanol (20 ml) was heated under reflux for 1.5 hr. The solvent was evaporated under reduced pressure, and the residue was acidified with 1N hydrochloric acid. The precipitated crystals were collected by filtration, and dried to give the object compound (1.86 g).

¹H-NMR (DMSO-d₆) δ 1.22-1.40 (4H, m), 1.74-1.84 (3H, m), 2.06-2.19 (1H, m), 3.81-3.90 (2H, m), 7.37-7.52 (5H, m), 7.90 (1H, s), 11.90 (1H, br s)

In the same manner as in Reference Example 71, the following compounds (Reference Examples 72-75) were obtained.

### Reference Example 72

### 1-{(1S,2S)-2-[(Methoxycarbonyl)amino]cyclohexyl}-5-phenyl-1H-imidazole-4-carboxylic acid

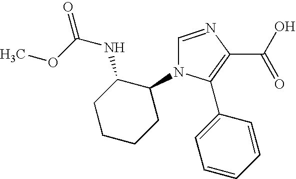

¹H-NMR (DMSO-d₆) δ 0.93-0.98 (1H, m), 1.28-1.34 (2H, m), 1.60-1.79 (5H, m), 3.44 (3H, s), 3.55-3.61 (1H, m), 3.90-3.93 (1H, m), 7.09-7.12 (1H, m), 7.31-7.49 (5H, m), 7.89 (1H, s), 11.74 (1H, br s)

### Reference Example 73

### 1-{(1S,2S)-2-[(Ethoxycarbonyl)amino]cyclohexyl}-5-phenyl-1H-imidazole-4-carboxylic acid

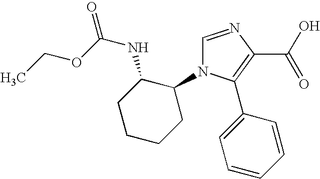

¹H-NMR (DMSO-d₆) δ 0.94-0.98 (1H, m), 1.08 (3H, t), 1.25-1.33 (2H, m), 1.60-1.79 (5H, m), 3.54-3.60 (1H, m), 3.85-3.91 (3H, m), 7.06-7.09 (1H, m), 7.32-7.49 (5H, m), 7.96 (1H, s), 11.80 (1H, br s)

### Reference Example 74

### 1-{(1S,2R)-2-[(Ethoxycarbonyl)amino]cyclohexyl}-5-phenyl-1H-imidazole-4-carboxylic acid

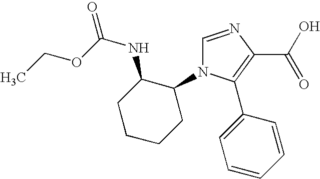

¹H-NMR (DMSO-d₆) δ 1.09 (3H, s), 1.20-1.34 (2H, m), 1.53-1.57 (2H, m), 1.73-1.84 (2H, m), 3.38 (2H, q), 3.69 (1H, br s), 3.88-4.00 (3H, m), 7.44-7.58 (5H, m), 8.81 (1H, s), 13.00 (1H, br s)

### Reference Example 75

### 1-[(1S,2R)-2-Fluorocyclohexyl]-5-phenyl-1H-imidazole-4-carboxylic acid

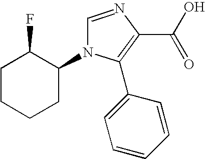

¹H-NMR (DMSO-d₆) δ 1.24-1.44 (4H, m), 1.74-1.82 (2H, m), 1.83-1.95 (1H, m), 2.08-2.20 (1H, m), 3.71-3.87 (1H, m), 4.73 (1H, d), 7.36-7.48 (5H, m), 7.91 (1H, d), 11.96 (1H, br s)

### Reference Example 76

### 1-[(1S,2R)-2-Hydroxycyclohexyl]-5-phenyl-1H-imidazole-4-carboxylic acid

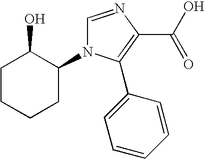

Ethyl 1-[(1S,2R)-2-hydroxycyclohexyl]-5-phenyl-1H-imidazole-4-carboxylate (6.1 g) was dissolved in ethanol-THF (1:1, 200 ml). 8N aqueous sodium hydroxide solution (10 ml) was added, and the mixture was stirred at 50° C. for 3 hr. The reaction mixture was concentrated under reduced pressure, and the residue was neutralized with 1N hydrochloric acid, subjected to DIAION HP-20 (manufactured by Mitsubishi Chemical), and washed with water. The fraction eluted with acetone was concentrated under reduced pressure to give the object compound (4.52 g) as an amorphous solid.

MS (ESI+, m/e) 287 (M+1)

### Reference Example 77

### 5-(3-Fluorophenyl)-1-[(1S,2S)-2-hydroxycyclohexyl]-1H-imidazole-4-carboxylic acid

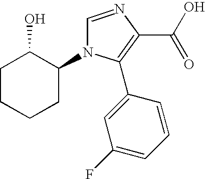

Methyl 5-(3-fluorophenyl)-1-[(1S,2S)-2-hydroxycyclohexyl]-1H-imidazole-4-carboxylate (1.05 g) was dissolved in methanol-THF (1:1, 20 ml). 8N aqueous sodium hydroxide solution (3 ml) was added, and the mixture was stirred at 50° C. for 3 hr. The reaction mixture was concentrated under reduced pressure, and the residue was neutralized with 1N hydrochloric acid, subjected to DIAION HP-20 (manufactured by Mitsubishi Chemical), and washed with water. The fraction eluted with acetone was concentrated under reduced pressure to give the object compound (981 mg) as an amorphous solid.

¹H-NMR (DMSO-d₆) δ 0.71-1.41 (5H, m), 1.41-1.99 (5H, m), 2.89-3.94 (2H, m), 6.88-7.67 (4H, m), 7.84 (1H, br s)

In the same manner as in Reference Example 77, the following compounds (Reference Examples 78-83) were obtained.

### Reference Example 78

### 1,5-Dicyclohexyl-1H-imidazole-4-carboxylic acid

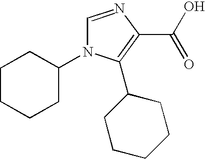

¹H-NMR (DMSO-d₆) δ 1.11-2.14 (20H, m), 3.10-3.33 (1H, m), 4.04-4.23 (1H, m), 8.08 (1H, s)

### Reference Example 79

### 1-Cyclohexyl-3-cyclopropyl-1H-imidazole-4-carboxylic acid

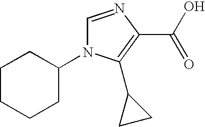

¹H-NMR (DMSO-d₆) δ 0.61 (2H, br s), 0.87 (2H, d), 1.07-2.02 (11H, m), 4.08 (1H, br s), 4.90 (1H, br s), 7.33 (1H, br s)

### Reference Example 80

### 5-(3-Fluorophenyl)-1-[(1S,2R)-2-hydroxycyclohexyl]-1H-imidazole-4-carboxylic acid

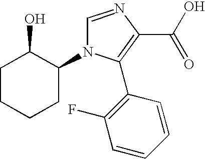

MS (ESI+, m/e) 305 (M+1)

### Reference Example 81

### 5-(3,5-Difluorophenyl)-1-[(1S,2R)-2-hydroxycyclohexyl]-1H-imidazole-4-carboxylic acid

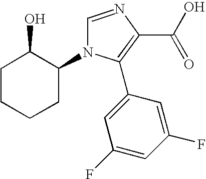

MS (ESI+, m/e) 323 (M+1)

### Reference Example 82

### 5-(2,3-Difluorophenyl)-1-[(1S,2S)-2-hydroxycyclohexyl]-1H-imidazole-4-carboxylic acid

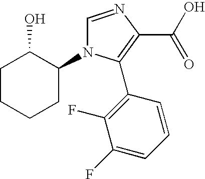

MS (ESI+, m/e) 323 (M+1)

### Reference Example 83

### 5-(4-Fluorophenyl)-1-[(1S,2S)-2-hydroxycyclohexyl]-1H-imidazole-4-carboxylic acid

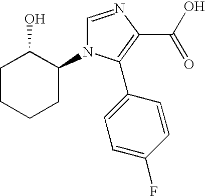

MS (ESI+, m/e) 305 (M+1)

### Reference Example 84

### 1-[(1R,2S)-2-Hydroxy-2-(methoxymethyl)cyclohexyl]-5-phenyl-1H-imidazole-4-carboxylic acid

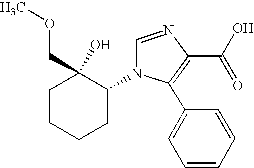

Ethyl 1-[(3S,4R)-1-oxaspiro[2.5]oct-4-yl]-5-phenyl-1H-imidazole-4-carboxylate (7.83 g) was dissolved in methanol (120 ml). Sodium methoxide (28% methanol solution, 23.1 ml) was added at room temperature, and the mixture was stirred at 60° C. for 15 hr. To the reaction mixture was added water (24 ml), and the mixture was further stirred at 60° C. for 6 hr. After cooling to room temperature, the mixture was neutralized (pH 7) with hydrochloric acid, and the solvent was evaporated under reduced pressure. The residue was dissolved in water, subjected to DIAION HP-20 (manufactured by Mitsubishi Chemical), and washed with water, and the fraction eluted with acetone was concentrated under reduced pressure to give the object compound (7.92 g) as an amorphous solid.

¹H-NMR (DMSO-d₆) δ 1.03 (1H, t), 1.26-1.44 (2H, m), 1.44-1.79 (4H, m), 1.96-2.14 (1H, m), 2.58-2.65 (1H, m), 2.68-2.77 (1H, m), 2.90-3.00 (3H, m), 3.62-3.73 (1H, m), 5.08 (1H, br s), 7.21-7.47 (5H, m), 7.95 (1H, s), 11.74 (1H, br s)

In the same manner as in Reference Example 84, the following compound (Reference Example 85) was obtained.

### Reference Example 85

### 5-(3-Fluorophenyl)-1-[(1R,2S)-2-hydroxy-2-(methoxymethyl)cyclohexyl]-1H-imidazole-4-carboxylic acid

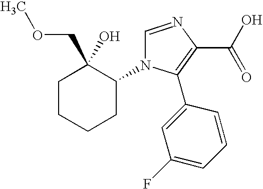

MS (ESI+, m/e) 349 (M+1)

### Reference Example 86

### Ethyl N-(tert-butoxycarbonyl)-2-fluoro-D-phenylalanyl-N-benzylglycinate

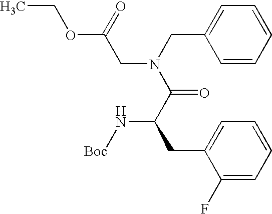

A solution of N-(tert-butoxycarbonyl)-2-fluoro-D-phenylalanine (999 mg), ethyl N-benzylglycinate (716 mg), WSC HCl (811 mg) and HOBt (524 mg) in DMF (18 ml) was stirred at room temperature for 15 hr, and poured into water, and the mixture was extracted with ethyl acetate. The extract was washed successively with 10% aqueous citric acid solution, water, saturated aqueous sodium hydrogen carbonate, water and saturated brine, and dried over anhydrous magnesium sulfate, and the solvent was evaporated under reduced pressure to give the object compound (1.62 g) as an amorphous solid.

MS (ESI+, m/e) 359 (M+1-“Boc”)

In the same manner as in Reference Example 86, the following compounds (Reference Examples 87-101) were obtained.

### Reference Example 87

### Ethyl N-(tert-butoxycarbonyl)-3-fluoro-D-phenylalanyl-N-benzylglycinate

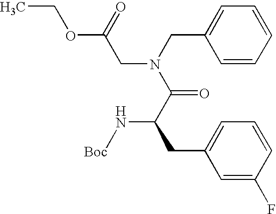

MS (ESI+, m/e) 359 (M+1-“Boc”)

### Reference Example 88

### Ethyl N-(tert-butoxycarbonyl)-4-fluoro-D-phenylalanyl-N-benzylglycinate

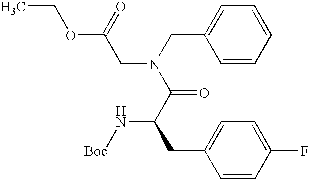

MS (ESI+, m/e) 359 (M+1-“Boc”)

### Reference Example 89

### Ethyl N-(tert-butoxycarbonyl)-3,4-difluoro-D-phenylalanyl-N-benzylglycinate

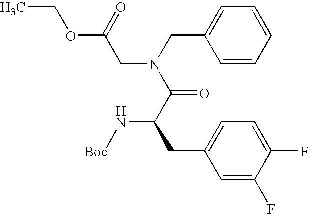

MS (ESI+, m/e) 377 (M+1-“Boc”)

### Reference Example 90

### Ethyl N-(tert-butoxycarbonyl)-3,5-difluoro-D-phenylalanyl-N-benzylglycinate

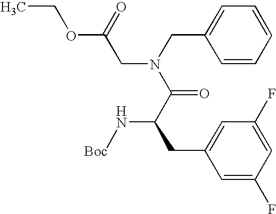

MS (ESI+, m/e) 377 (M+1-“Boc”)

### Reference Example 91

### Ethyl N-(tert-butoxycarbonyl)-2,4,5-trifluoro-D-phenylalanyl-N-benzylglycinate

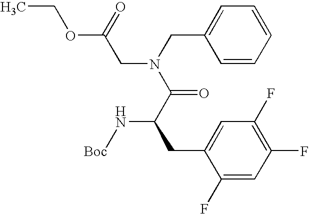

MS (ESI+, m/e) 395 (M+1-“Boc”)

### Reference Example 92

### Ethyl N-(tert-butoxycarbonyl)-2-(trifluoromethyl)-D-phenylalanyl-N-benzylglycinate

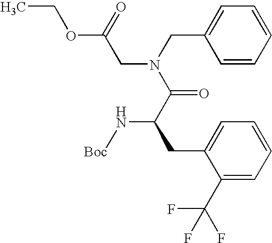

MS (ESI+, m/e) 409 (M+1-“Boc”)

### Reference Example 93

### Ethyl N-(tert-butoxycarbonyl)-3-(trifluoromethyl)-D-phenylalanyl-N-benzylglycinate

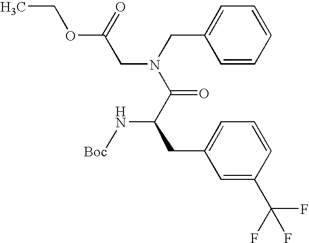

MS (ESI+, m/e) 409 (M+1-“Boc”)

### Reference Example 94

### Ethyl N-(tert-butoxycarbonyl)-4-(trifluoromethyl)-D-phenylalanyl-N-benzylglycinate

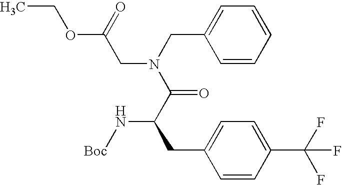

MS (ESI+, m/e) 409 (M+1-“Boc”)

### Reference Example 95

### Ethyl N-(tert-butoxycarbonyl)-O-methyl-D-tyrosyl-N-benzylglycinate

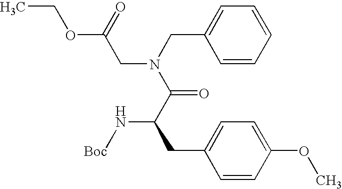

MS (ESI+, m/e) 371 (M+1-“Boc”)

### Reference Example 96

### Ethyl 4-bromo-N-(tert-butoxycarbonyl)-D-phenylalanyl-N-benzylglycinate

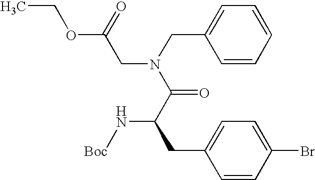

MS (ESI+, m/e) 519 (M+1)

### Reference Example 97

### Ethyl N-benzyl-N-[(2R)-2-[(tert-butoxycarbonyl)amino]-2-(2,3-dihydro-1H-inden-2-yl)acetyl]glycinate

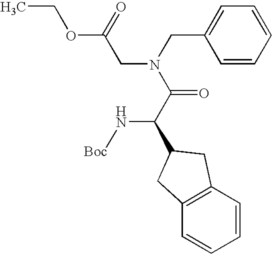

MS (ESI+, m/e) 367 (M+1-“Boc”)

### Reference Example 98

### Ethyl N-(tert-butoxycarbonyl)-4-methyl-D-leucyl-N-benzylglycinate

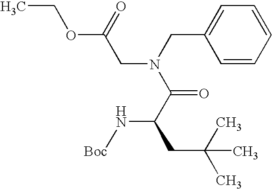

MS (ESI+, m/e) 421 (M+1)

### Reference Example 99

### Ethyl N-(tert-butoxycarbonyl)-D-leucyl-N-benzylglycinate

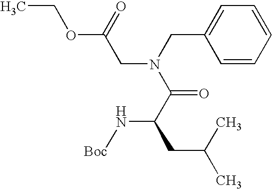

MS (ESI+, m/e) 307 (M+1-“Boc”)

### Reference Example 100

### Ethyl N-(tert-butoxycarbonyl)-3-cyclohexyl-D-alanyl-N-benzylglycinate

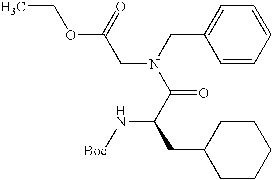

¹H-NMR (CDCl₃) δ 0.74-1.88 (25H, m), 3.70-3.89 (1H, m), 4.09-4.29 (2H, m), 4.42-4.61 (2H, m), 4.74-4.92 (2H, m), 5.10-5.18 (1H, m), 7.18-7.38 (5H, m)

### Reference Example 101

### Ethyl N-(tert-butoxycarbonyl)-D-tyrosyl-N-benzylglycinate

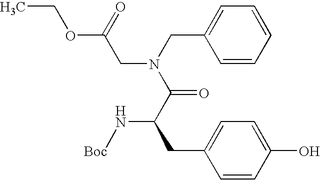

¹H-NMR (CDCl₃) δ 1.11-1.52 (12H, m), 3.66-4.26 (5H, m), 4.36-4.78 (3H, m), 4.83-5.13 (1H, m), 5.22-5.37 (1H, m), 5.65 (1H, br s), 6.61-7.49 (10H, m)

### Reference Example 102

### Ethyl N-(tert-butoxycarbonyl)-3-(pyridin-3-yl)-D-alanyl-N-benzylglycinate

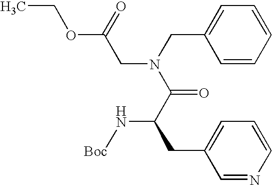

A solution of N-(tert-butoxycarbonyl)-3-(pyridin-3-yl)-D-alanine (5.00 g), ethyl N-benzylglycinate (3.81 g), WSC.HCl (4.32 g) and HOBt (2.79 g) in DMF (85 ml) was stirred at room temperature for 15 hr, and poured into saturated aqueous sodium hydrogen carbonate, and the mixture was extracted with ethyl acetate. The extract was washed successively with water and saturated brine, and dried over anhydrous magnesium sulfate, and the solvent was evaporated under reduced pressure to give the object compound (8.28 g) as an oil (it was allowed to crystallization at low temperature).

MS (ESI+, m/e) 442 (M+1)

In the same manner as in Reference Example 102, the following compounds (Reference Examples 103-106) were obtained.

### Reference Example 103

### Ethyl N-(tert-butoxycarbonyl)-3-(pyridin-2-yl)-D-alanyl-N-benzylglycinate

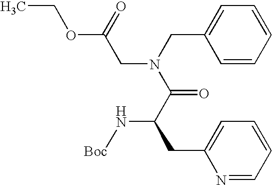

MS (ESI+, m/e) 442 (M+1)

### Reference Example 104

### Ethyl N-(tert-butoxycarbonyl)-3-(pyridin-4-yl)-D-alanyl-N-benzylglycinate

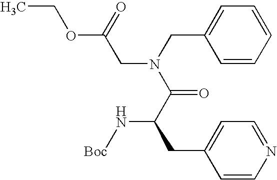

MS (ESI+, m/e) 442 (M+1)

### Reference Example 105

### Ethyl N-(tert-butoxycarbonyl)-D-tryptophyl-N-benzylglycinate

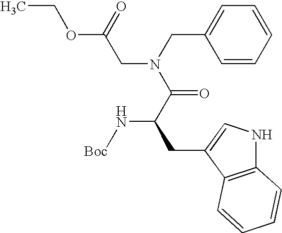

MS (ESI+, m/e) 480 (M+1)

### Reference Example 106

### Ethyl N-(tert-butoxycarbonyl)-D-histidyl-N-benzylglycinate

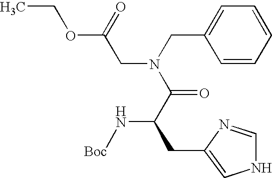

MS (ESI+, m/e) 431 (M+1)

### Reference Example 107

### Ethyl N-(tert-butoxycarbonyl)-2,4-dichloro-D-phenylalanylglycinate

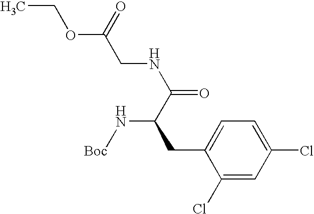

A mixture of N-(tert-butoxycarbonyl)-2,4-dichloro-D-phenylalanine (5.00 g), glycine ethyl ester hydrochloride (2.19 g), WSC.HCl (3.44 g), HOBt (2.22 g), triethylamine (1.82 g) and DMF (70 ml) was stirred at room temperature for 15 hr, and poured into water, and the mixture was extracted with ethyl acetate. The extract was washed successively with 10% aqueous citric acid solution, water, saturated aqueous sodium hydrogen carbonate, water and saturated brine, and dried over anhydrous magnesium sulfate. The solvent was evaporated under reduced pressure, and the crystals were collected by filtration to give the object compound (5.69 g).

¹H-NMR (CDCl₃) δ 1.28 (3H, t), 1.36 (9H, s), 3.00-3.07 (1H, m), 3.31 (1H, dd), 3.95 (1H, dd), 4.05 (1H, dd), 4.21 (2H, q), 4.48-4.50 (1H, m), 5.05-5.07 (1H, m), 6.52 (1H, br s), 7.15-7.21 (2H, m), 7.38 (1H, s)

MS (ESI+, m/e) 319 (M+1-“Boc”)

In the same manner as in Reference Example 107, the following compound (Reference Example 108) was obtained.

### Reference Example 108

### Ethyl N-(tert-butoxycarbonyl)-3-(1,3-thiazol-4-yl)-D-alanylglycinate

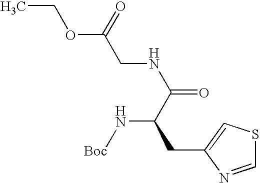

MS (ESI+, m/e) 358 (M+1)

### Reference Example 109

### (3R)-1-Benzyl-3-(2-fluorobenzyl)piperazine-2,5-dione

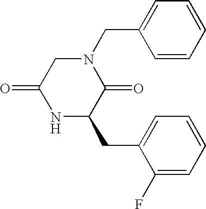

To a solution of ethyl N-(tert-butoxycarbonyl)-2-fluoro-D-phenylalanyl-N-benzylglycinate (1.58 g) in dichloromethane (0.9 ml) was added TFA (9 ml), and the mixture was stirred at room temperature for 1 hr. The reaction mixture was concentrated under reduced pressure, and the residue was diluted with toluene. The mixture was again concentrated under reduced pressure to remove TFA. The residue was dissolved in dichloromethane (15 ml), triethylamine (3 ml) was added, and the mixture was stirred at room temperature for 2 hr, and concentrated under reduced pressure, and the residue was dissolved in ethyl acetate-THF (4:1, 100 ml). The solution was washed successively with 10% aqueous citric acid solution, water, saturated aqueous sodium hydrogen carbonate, water and saturated brine, and dried over anhydrous magnesium sulfate. The solvent was evaporated under reduced pressure, and the crystals were collected by filtration to give the object compound (950 mg).

¹H-NMR (CDCl₃) δ 3.14 (1H, dd), 3.22 (1H, d), 3.38 (1H, dd), 3.63 (1H, d), 4.33-4.37 (1H, m), 4.49 (1H, d), 4.56 (1H, d), 6.18 (1H, s), 6.93-7.06 (2H, m), 7.14 (1H, dt), 7.20-7.28 (3H, m), 7.31-7.36 (3H, m)

MS (ESI+, m/e) 313 (M+1)

In the same manner as in Reference Example 109, the following compounds (Reference Examples 110-124) were obtained.

### Reference Example 110

### (3R)-1-Benzyl-3-(3-fluorobenzyl)piperazine-2,5-dione

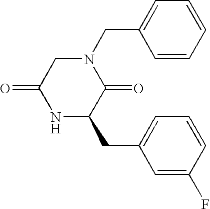

¹H-NMR (CDCl₃) δ 3.12-3.24 (3H, m), 3.61 (1H, d), 4.33-4.37 (1H, m), 4.49 (1H, d), 4.56 (1H, d), 6.51 (1H, s), 6.92-6.99 (3H, m), 7.19-7.24 (3H, m), 7.30-7.37 (3H, m)

MS (ESI+, m/e) 313 (M+1)

### Reference Example 111

### (3R)-1-Benzyl-3-(4-fluorobenzyl)piperazine-2,5-dione

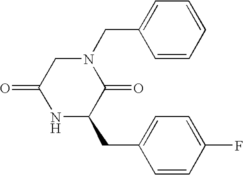

¹H-NMR (CDCl₃) δ 3.02 (1H, d), 3.08 (1H, dd), 3.21 (1H, dd), 3.55 (1H, d), 4.32-4.36 (1H, m), 4.39 (1H, d), 4.55 (1H, d), 6.68 (1H, s), 6.84-6.91 (2H, m), 7.07-7.18 (4H, m), 7.30-7.33 (3H, m)

MS (ESI+, m/e) 313 (M+1)

### Reference Example 112

### (3R)-1-Benzyl-3-(3,4-difluorobenzyl)piperazine-2,5-dione

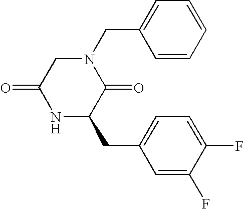

¹H-NMR (CDCl₃) δ 3.09 (1H, dd), 3.20 (1H, dd), 3.23 (1H, d), 3.63 (1H, d), 4.32-4.37 (1H, m), 4.41 (1H, d), 4.62 (1H, d), 6.85 (1H, s), 6.87-6.89 (1H, m), 6.94-7.06 (2H, m), 7.16-7.19 (2H, m), 7.31-7.36 (3H, m)

MS (ESI+, m/e) 331 (M+1)

### Reference Example 113

### (3R)-1-Benzyl-3-(3,5-difluorobenzyl)piperazine-2,5-dione

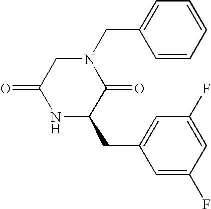

¹H-NMR (CDCl₃) δ 3.11-3.23 (2H, m), 3.38 (1H, d), 3.68 (1H, d), δ 4.33-4.37 (1H, m), 4.48 (1H, d), 4.61 (1H, d), 6.67-6.79 (4H, m), 7.17-7.20 (2H, m), 7.28-7.37 (3H, m)

MS (ESI+, m/e) 331 (M+1)

### Reference Example 114

### (3R)-1-Benzyl-3-(2,4,5-trifluorobenzyl)piperazine-2,5-dione

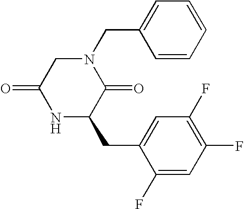

¹H-NMR (CDCl₃) δ 3.14 (1H, dd), 3.27 (1H, dd), 3.42 (1H, d), 3.70 (1H, d), 4.36-4.39 (1H, m), 4.44 (1H, d), 4.65 (1H, d), 6.55 (1H, s), 6.85-6.93 (1H, m), 7.01-7.10 (1H, m), 7.17-7.20 (2H, m), 7.30-7.35 (3H, m)

MS (ESI+, m/e) 349 (M+1)

### Reference Example 115

### (3R)-1-Benzyl-3-[2-(trifluoromethyl)benzyl]piperazine-2,5-dione

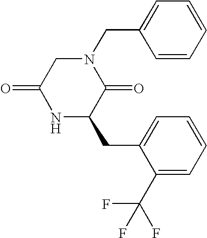

MS (ESI+, m/e) 363 (M+1)

### Reference Example 116

### (3R)-1-Benzyl-3-[3-(trifluoromethyl)benzyl]piperazine-2,5-dione

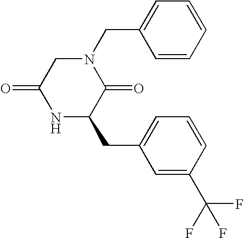

¹H-NMR (CDCl₃) δ 3.13 (1H, d), 3.21-3.31 (2H, m), 3.60 (1H, d), 4.37 (1H, d), 4.37-4.41 (1H, m), 4.62 (1H, d), 6.66 (1H, s), 7.15-7.20 (2H, m), 7.28-7.39 (5H, m), 7.50-7.56 (2H, m)

MS (ESI+, m/e) 363 (M+1)

### Reference Example 117

### (3R)-1-Benzyl-3-[4-(trifluoromethyl)benzyl]piperazine-2,5-dione

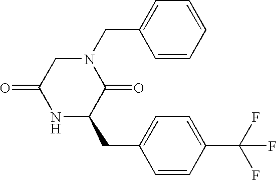

¹H-NMR (CDCl₃) δ 3.07 (1H, d), 3.18 (1H, dd), 3.28 (1H, dd), 3.58 (1H, d), 4.29 (1H, d), 4.37-4.41 (1H, m), 4.68 (1H, d), 6.50 (1H, s), 7.16-7.19 (2H, m), 7.24-7.27 (2H, m), 7.32-7.35 (3H, m), 7.43 (2H, d)

MS (ESI+, m/e) 363 (M+1)

### Reference Example 118

### (3R)-1-Benzyl-3-(4-methoxybenzyl)piperazine-2,5-dione

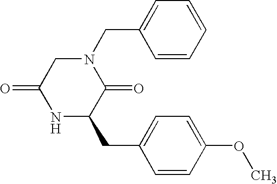

¹H-NMR (CDCl₃) δ 2.97 (1H, d), 3.06 (1H, dd), 3.15 (1H, dd), 3.51 (1H, d), 3.75 (3H, s), 4.28-4.32 (1H, m), 4.43 (1H, d), 4.51 (1H, d), 6.43 (1H, s), 6.72 (2H, d), 7.04 (2H, d), 7.16-7.20 (2H, m), 7.29-7.34 (3H, m)

MS (ESI+, m/e) 325 (M+1)

### Reference Example 119

### (3R)-1-Benzyl-3-(4-bromobenzyl)piperazine-2,5-dione

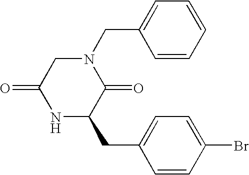

¹H-NMR (CDCl₃) δ 3.06 (1H, dd), 3.07 (1H, d), 3.18 (1H, dd), 3.56 (1H, d), 4.32-4.36 (1H, m), 4.35 (1H, d), 4.60 (1H, d), 6.63 (1H, s), 7.00 (2H, d), 7.14-7.17 (2H, m), 7.27-7.36 (5H, m)

MS (ESI+, m/e) 373 (M+1)

### Reference Example 120

### (3R)-1-Benzyl-3-(2,3-dihydro-1H-inden-2-yl)piperazine-2,5-dione

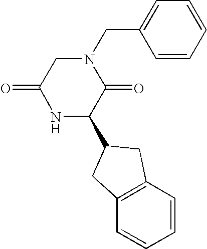

¹H-NMR (CDCl₃) δ 2.76 (1H, dd), 2.88-3.16 (4H, m), 3.78 (1H, d), 3.88 (1H, d), 4.15 (1H, dd), 4.48 (1H, d), 4.75 (1H, d), 6.87 (1H, s), 7.10-7.20 (4H, m), 7.25-7.40 (5H, m)

MS (ESI+, m/e) 321 (M+1)

### Reference Example 121

### (3R)-1-Benzyl-3-(2,2-dimethylpropyl)piperazine-2,5-dione

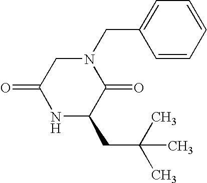

¹H-NMR (CDCl₃) δ 1.01 (9H, s), 1.55 (1H, dd), 2.11 (1H, dd), 3.79 (1H, d), 3.87 (1H, dd), 4.06 (1H, dt), 4.54 (1H, d), 4.63 (1H, d), 6.32 (1H, br s), 7.23-7.26 (2H, m), 7.30-7.38 (3H, m)

MS (ESI+, m/e) 275 (M+1)

### Reference Example 122

### (3R)-1-Benzyl-3-isobutylpiperazine-2,5-dione

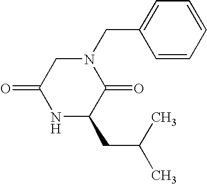

MS (ESI+, m/e) 261 (M+1)

### Reference Example 123

### (3R)-1-Benzyl-3-(cyclohexylmethyl)piperazine-2,5-dione

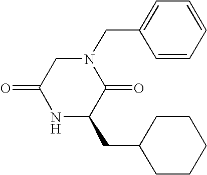

¹H-NMR (CDCl₃) δ 0.93-1.05 (2H, m), 1.12-1.29 (3H, m), 1.40-1.46 (1H, m), 1.57-1.89 (8H, m), 3.76-3.89 (2H, m), 4.06-4.12 (1H, m), 4.59 (2H, dd), 6.98 (1H, s), 7.24-7.38 (5H, m)

### Reference Example 124

### (3R)-1-Benzyl-3-(4-hydroxybenzyl)piperazine-2,5-dione

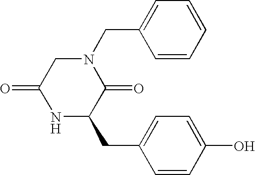

¹H-NMR (DMSO-d₆) δ 2.70-2.82 (1H, m), 2.99-3.11 (1H, m), 3.32-3.43 (2H, m), 4.14-4.26 (2H, m), 4.55 (1H, d), 6.52 (2H, d), 6.83 (2H, d), 7.11 (2H, m), 7.23-7.39 (3H, m), 8.23-8.31 (1H, m), 9.26 (1H, s)

### Reference Example 125

### (3R)-1-Benzyl-3-(pyridin-3-ylmethyl)piperazine-2,5-dione

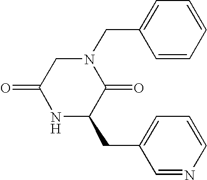

To a solution of ethyl N-(tert-butoxycarbonyl)-3-(pyridin-3-yl)-D-alanyl-N-benzylglycinate (8.27 g) in dichloromethane (5 ml) was added TFA (50 ml), and the mixture was stirred at room temperature for 50 min. The reaction mixture was concentrated under reduced pressure, and the residue was diluted with toluene. The mixture was again concentrated under reduced pressure to remove TFA. The residue was dissolved in dichloromethane (75 ml), triethylamine (15 ml) was added thereto, the mixture was stirred at room temperature for 2 hr, and concentrated under reduced pressure, and the residue was dissolved in chloroform (about 200 ml). The solution was washed successively with water and saturated brine, and dried over anhydrous magnesium sulfate. The solvent was evaporated under reduced pressure, and the crystals were collected by filtration to give the object compound (4.14 g).

¹H-NMR (CDCl₃) δ 3.14 (1H, dd), 3.16 (1H, d), 3.26 (1H, dd), 3.60 (1H, d), 4.37-4.41 (1H, m), 4.50 (2H, s), 7.12-7.19 (3H, m), 7.24 (1H, s), 7.28-7.33 (3H, m), 7.50 (1H, dt), 8.48-8.50 (2H, m)

MS (ESI+, m/e) 296 (M+1)

In the same manner as in Reference Example 125, the following compounds (Reference Examples 126-129) were obtained.

### Reference Example 126

### (3R)-1-Benzyl-3-(pyridin-2-ylmethyl)piperazine-2,5-dione

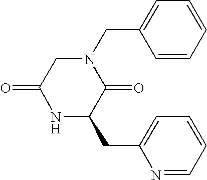

MS (ESI+, m/e) 296 (M+1)

### Reference Example 127

### (3R)-1-Benzyl-3-(pyridin-4-ylmethyl)piperazine-2,5-dione

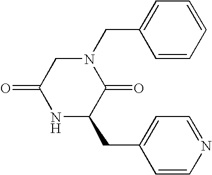

¹H-NMR (CDCl₃) δ 3.14 (1H, dd), 3.22 (1H, dd), 3.26 (1H, d), 3.64 (1H, d), 4.38-4.43 (1H, m), 4.40 (1H, d), 4.61 (1H, d), 6.91 (1H, s), 7.10 (2H, d), 7.16-7.21 (2H, m), 7.31-7.39 (3H, m), 8.44 (2H, d)

MS (ESI+, m/e) 296 (M+1)

### Reference Example 128

### (3R)-1-Benzyl-3-(1H-indol-3-ylmethyl)piperazine-2,5-dione

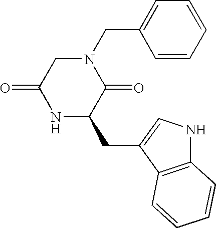

¹H-NMR (CDCl₃) δ 3.02 (1H, d), 3.36 (2H, d), 3.50 (1H, d), 4.12 (1H, d), 4.35-4.39 (1H, m), 4.59 (1H, d), 6.31 (1H, s), 6.98 (1H, d), 7.02-7.05 (2H, m), 7.13-7.27 (5H, m), 7.37 (1H, d), 7.64 (1H, d), 8.21 (1H, s)

MS (ESI+, m/e) 334 (M+1)

### Reference Example 129

### (3R)-1-Benzyl-3-(1H-imidazol-4-ylmethyl)piperazine-2,5-dione

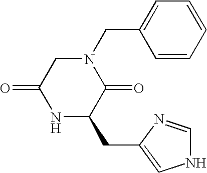

¹H-NMR (CDCl₃) δ 3.18 (1H, dd), 3.30 (1H, dd), 3.42 (1H, d), 3.70 (1H, d), 4.34-4.37 (1H, m), 4.50 (1H, d), 4.59 (1H, d), 6.83 (1H, s), 7.18-7.22 (2H, m), 7.28-7.36 (4H, m), 7.63 (1H, s), 8.11 (1H, s)

MS (ESI+, m/e) 285 (M+1)

### Reference Example 130

### (3R)-3-(2,4-Dichlorobenzyl)piperazine-2,5-dione

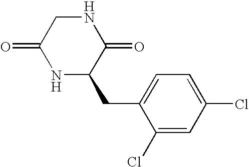

Ethyl N-(tert-butoxycarbonyl)-2,4-dichloro-D-phenylalanylglycinate (5.68 g) was suspended in dichloromethane (4 ml). TFA (40 ml) was added, and the mixture was stirred at room temperature for 50 min. The reaction mixture was concentrated under reduced pressure, and the residue was diluted with toluene. The mixture was again concentrated under reduced pressure to remove TFA. The residue was dissolved in ethanol (60 ml), triethylamine (12 ml) was added, and the mixture was heated under reflux for 15 hr. The reaction mixture was concentrated under reduced pressure, and the crystals were collected by filtration to give the object compound (3.40 g).

¹H-NMR (DMSO-d₆) δ 3.06 (1H, dd), 3.17 (1H, dd), 3.53 (1H, dd), 3.64 (1H, d), 3.94-3.98 (1H, m), 7.32 (1H, d), 7.39 (1H, dd), 7.58 (1H, d), 8.07 (2H, s)

MS (ESI+, m/e) 273 (M+1)

### Reference Example 131

### (3R)-3-(1,3-Thiazol-4-ylmethyl)piperazine-2,5-dione

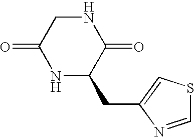

Ethyl N-(tert-butoxycarbonyl)-3-(1,3-thiazol-4-yl)-D-alanylglycinate (5.6 g) was dissolved in dichloromethane (5 ml). TFA (15 ml) was added, and the mixture was stirred at room temperature for 1 hr. The reaction mixture was concentrated under reduced pressure, and the residue was diluted with toluene. The mixture was again concentrated under reduced pressure to remove TFA. The residue was dissolved in methanol (40 ml), triethylamine (8 ml) was added thereto, and the mixture was stirred at room temperature for 12 hr. The reaction mixture was concentrated under reduced pressure, and the residue was dissolved in ethyl acetate-THF (4:1, 250 ml). The solution was washed successively with 10% aqueous citric acid solution, water, saturated aqueous sodium hydrogen carbonate, water and saturated brine, and dried over anhydrous magnesium sulfate, and the solvent was evaporated under reduced pressure. The residue was subjected to silica gel column chromatography, and the fraction eluted with ethyl acetate-methanol (1:1) was concentrated under reduced pressure to give the object compound (2.4 g) as an amorphous solid.

MS (ESI+, m/e) 212 (M+1)

### Reference Example 132

### Benzyl 3-[(2R)-4-benzyl-3,6-dioxopiperazin-2-yl]propionate

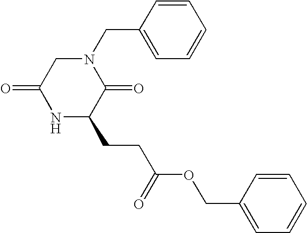

A solution of (2R)-5-(benzyloxy)-2-[(tert-butoxycarbonyl)amino]-5-oxopentanoic acid (50 g), ethyl N-benzylglycinate (28.6 g), WSC.HCl (34 g) and HOBt (25 g) in DMF (300 ml) was stirred at room temperature for 12 hr, and poured into aqueous sodium bicarbonate, and the mixture was extracted with ethyl acetate. The extract was washed successively with 10% aqueous citric acid solution and saturated brine, and dried over anhydrous sodium sulfate, and the solvent was evaporated under reduced pressure. The residue was dissolved in chloroform (150 ml), TFA (15 ml) was added thereto, and the mixture was stirred at room temperature for 1 hr. The reaction mixture was concentrated under reduced pressure, and the residue was diluted with toluene. The mixture was again concentrated under reduced pressure. The residue was dissolved in chloroform (400 ml), triethylamine (70 ml) was added thereto, and the mixture was stirred at room temperature for 12 hr. The reaction mixture was washed successively with water, 10% aqueous citric acid solution, water, saturated aqueous sodium hydrogen carbonate, water and saturated brine, and dried over anhydrous sodium sulfate, and the solvent was evaporated under reduced pressure. To the residue was added ethyl acetate-hexane (1:1), and the precipitated crystals were collected by filtration to give the object compound (57 g).

MS (ESI+, m/e) 367 (M+1)

### Reference Example 133

### (3R)-1-Benzyl-3-(2-fluorobenzyl)piperazine

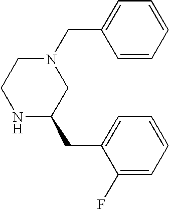

A mixture of (3R)-1-benzyl-3-(2-fluorobenzyl)piperazine-2,5-dione (942 mg) and THF (25 ml) was ice-cooled, and lithium aluminum hydride (458 mg) was added by small portions. The mixture was stirred at room temperature for 30 min, and then at 60° C. for 1.5 hr. The mixture was cooled to −78° C., and ethanol-ethyl acetate (1:1, 3 ml) and 1N aqueous sodium hydroxide solution (6 ml) were successively added dropwise. After the completion of the dropwise addition, the mixture was stirred at room temperature for 40 min. The insoluble material was filtered, and washed with ethyl acetate. The filtrate was washed with saturated brine, and dried over anhydrous magnesium sulfate, and the solvent was evaporated under reduced pressure. The residue was subjected to silica gel column chromatography, and the fraction eluted with ethyl acetate-methanol (1:0-10:1) was concentrated under reduced pressure to give the object compound (595 mg) as an oil.

¹H-NMR (CDCl₃) δ 1.57 (1H, br s), 1.90 (1H, t), 2.06 (1H, dt), 2.61 (1H, dd), 2.68-2.85 (4H, m), 2.92 (1H, dt), 3.00-3.06 (1H, m), 3.44 (1H, d), 3.55 (1H, d), 6.98-7.07 (2H, m), 7.15-7.32 (7H, m)

MS (ESI+, m/e) 285 (M+1)

In the same manner as in Reference Example 133, the following compounds (Reference Examples 134-146) were obtained.

### Reference Example 134

### (3R)-1-Benzyl-3-(3-fluorobenzyl)piperazine

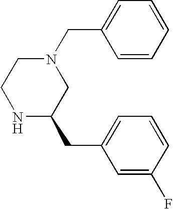

¹H-NMR (CDCl₃) δ 1.65 (1H, br s), 1.88 (1H, dd), 2.08 (1H, dt), 2.54 (1H, dd), 2.66-3.03 (6H, m), 3.46 (1H, d), 3.53 (1H, d), 6.87-6.97 (3H, m), 7.20-7.32 (6H, m)

MS (ESI+, m/e) 285 (M+1)

### Reference Example 135

### (3R)-1-Benzyl-3-(4-fluorobenzyl)piperazine

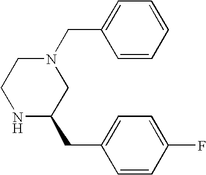

¹H-NMR (CDCl₃) δ 1.62 (1H, br s), 1.86 (1H, t), 2.03-2.11 (1H, m), 2.51 (1H, dd), 2.64-2.99 (6H, m), 3.46 (1H, d), 3.53 (1H, d), 6.49-7.00 (2H, m), 7.12-7.18 (2H, m), 7.21-7.32 (5H, m)

MS (ESI+, m/e) 285 (M+1)

### Reference Example 136

### (3R)-1-Benzyl-3-(3,4-difluorobenzyl)piperazine

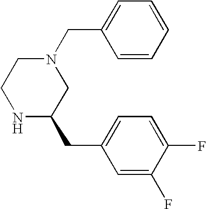

¹H-NMR (CDCl₃) δ 1.61 (1H, br s), 1.85 (1H, t), 2.07 (1H, dt), 2.50 (1H, dd), 2.65 (1H, dd), 2.71-2.84 (3H, m), 2.89-2.99 (2H, m), 3.46 (1H, d), 3.53 (1H, d), 6.86-6.90 (1H, m), 6.95-7.10 (2H, m), 7.22-7.32 (5H, m)

MS (ESI+, m/e) 303 (M+1)

### Reference Example 137

### (3R)-1-Benzyl-3-(2,4,5-trifluorobenzyl)piperazine

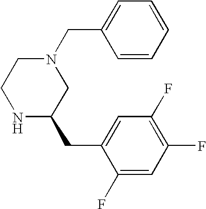

MS (ESI+, m/e) 321 (M+1)

### Reference Example 138

### (3R)-1-Benzyl-3-[2-(trifluoromethyl)benzyl]piperazine

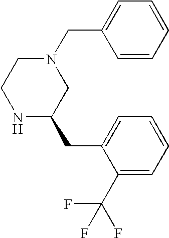

MS (ESI+, m/e) 335 (M+1)

### Reference Example 139

### (3R)-1-Benzyl-3-[3-(trifluoromethyl)benzyl]piperazine

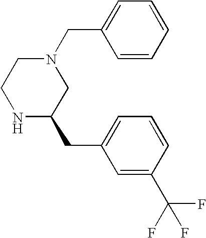

¹H-NMR (CDCl₃) δ 1.61 (1H, br s), 1.88 (1H, t), 2.08 (1H, dt), 2.61 (1H, dd), 2.73-2.85 (4H, m), 2.92 (1H, dt), 2.97-3.06 (1H, m), 3.47 (1H, d), 3.53 (1H, d), 7.21-7.48 (9H, m)

MS (ESI+, m/e) 335 (M+1)

### Reference Example 140

### (3R)-1-Benzyl-3-[4-(trifluoromethyl)benzyl]piperazine

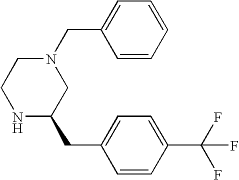

¹H-NMR (CDCl₃) δ 1.59 (1H, br s), 1.89 (1H, t), 2.08 (1H, dt), 2.61 (1H, dd), 2.71-2.84 (4H, m), 2.91 (1H, dt), 2.97-3.06 (1H, m), 3.47 (1H, d), 3.53 (1H, d), 7.21-7.31 (8H, m), 7.54 (1H, d)

MS (ESI+, m/e) 335 (M+1)

### Reference Example 141

### (3R)-1-Benzyl-3-(4-methoxybenzyl)piperazine

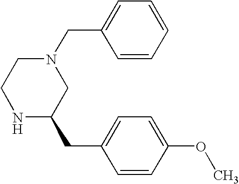

¹H-NMR (CDCl₃) δ 1.64 (1H, br s), 1.87 (1H, t), 2.07 (1H, dt), 2.47 (1H, dd), 2.65 (1H, dd), 2.71-2.95 (5H, m), 3.46 (1H, d), 3.54 (1H, d), 3.79 (3H, s), 6.83 (2H, d), 7.11 (2H, d), 7.23-7.32 (5H, m)

MS (ESI+, m/e) 297 (M+1)

### Reference Example 142

### (3R)-1-Benzyl-3-(2,3-dihydro-1H-inden-2-yl)piperazine

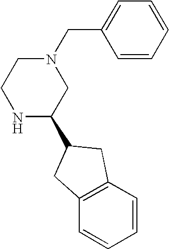

¹H-NMR (CDCl₃) δ 1.53 (1H, br s), 1.86 (1H, t), 2.05 (1H, dt), 2.33-2.45 (1H, m), 2.62-3.10 (9H, m), 3.46 (1H, d), 3.58 (1H, d), 7.08-7.32 (9H, m)

MS (ESI+, m/e) 293 (M+1)

### Reference Example 143

### (3R)-1-Benzyl-3-(2,2-dimethylpropyl)piperazine

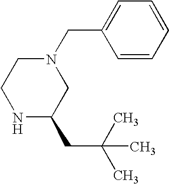

¹H-NMR (CDCl₃) δ 0.91 (9H, s), 1.18-1.20 (2H, m), 1.62 (1H, br s), 1.75 (1H, t), 1.94-2.02 (1H, m), 2.70-2.92 (5H, m), 3.44 (1H, d), 3.53 (1H, d), 7.22-7.31 (5H, m)

MS (ESI+, m/e) 247 (M+1)

### Reference Example 144

### (3R)-1-Benzyl-3-isobutylpiperazine

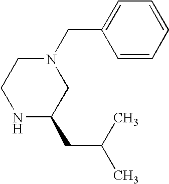

MS (ESI+, m/e) 233 (M+1)

### Reference Example 145

### (3R)-1-Benzyl-3-(cyclohexylmethyl)piperazine

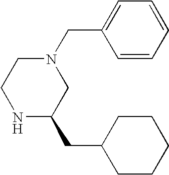

MS (ESI+, m/e) 273 (M+1)

### Reference Example 146

### 4-{[(2R)-4-Benzylpiperazin-2-yl]methyl}phenol

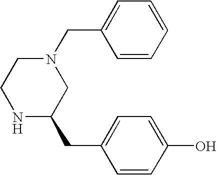

¹H-NMR (DMSO-d₆) δ 1.52-2.88 (8H, m), 3.08-3.73 (4H, m), 6.64 (2H, d), 6.94 (2H, d), 7.16-7.35 (5H, m), 9.17 (1H, br s)

MS (ESI+, m/e) 283 (M+1)

### Reference Example 147

### (3R)-1-Benzyl-3-(3,5-difluorobenzyl)piperazine

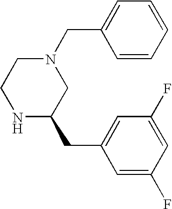

(3R)-1-Benzyl-3-(3,5-difluorobenzyl)piperazine-2,5-dione (4.36 g) was dissolved in THF (55 ml), and borane-THF (1.0M THF solution, 105.6 ml) was added dropwise at room temperature over 15 min. The mixture was stirred at room temperature for 1 hr, and then at 60° C. for 15 hr. The reaction mixture was ice-cooled, and water (6 ml) was added dropwise over 5 min. The mixture was stirred at room temperature for 10 min, and the reaction mixture was concentrated under reduced pressure. To the residue was added 2N hydrochloric acid (60 ml), and the mixture was stirred at 50° C. for 30 min. The reaction mixture was ice-cooled again, basified with 8N aqueous sodium hydroxide solution to weak basic (pH 8-9), and extracted with ethyl acetate. The extract was washed with saturated brine, and dried over anhydrous magnesium sulfate, and the solvent was evaporated under reduced pressure. The residue was subjected to silica gel column chromatography, and the fraction eluted with ethyl acetate-hexane-methanol (1:1:0-20:0:1) was concentrated under reduced pressure to give the object compound (1.81 g) as an oil (it was allowed to crystallization at low temperature).

¹H-NMR (CDCl₃) δ 1.64 (1H, br s), 1.86 (1H, t), 2.08 (1H, dt), 2.54 (1H, dd), 2.64-3.01 (6H, m), 3.47 (1H, d), 3.53 (1H, d), 6.61-6.74 (3H, m), 7.24-7.32 (5H, m)

MS (ESI+, m/e) 303 (M+1)

In the same manner as in Reference Example 147, the following compounds (Reference Examples 148-152) were obtained.

### Reference Example 148

### (3R)-1-Benzyl-3-(pyridin-2-ylmethyl)piperazine

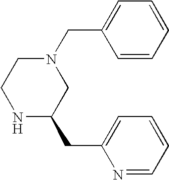

¹H-NMR (CDCl₃) δ 2.14-2.28 (3H, m), 2.78-2.93 (5H, m), 3.19-3.32 (1H, m), 3.50 (1H, d), 3.67 (1H, d), 3.81 (1H, s), 7.04-7.19 (2H, m), 7.22-7.37 (5H, m), 7.58 (1H, td), 8.53 (1H, dd).

MS (ESI+, m/e) 268 (M+1)

### Reference Example 149

### (3R)-1-Benzyl-3-(pyridin-4-ylmethyl)piperazine

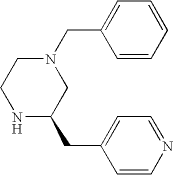

¹H-NMR (CDCl₃) δ 1.60 (1H, br s), 1.88 (1H, t), 2.09 (1H, dt), 2.56 (1H, dd), 2.66-3.07 (6H, m), 3.47 (1H, d), 3.53 (1H, d), 7.12 (2H, d), 7.24-7.35 (5H, m), 8.51 (2H, d)

MS (ESI+, m/e) 268 (M+1)

### Reference Example 150

### 3-{[(2R)-4-Benzylpiperazin-2-yl]methyl}-1H-indole

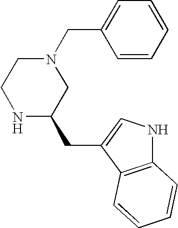

MS (ESI+, m/e) 306 (M+1)

### Reference Example 151

### (3R)-1-Benzyl-3-(1H-imidazol-4-ylmethyl)piperazine

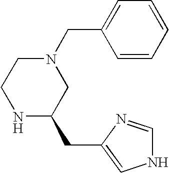

¹H-NMR (CDCl₃) δ 1.83 (1H, t), 2.06 (1H, dt), 2.56 (1H, dd), 2.64-3.07 (7H, m), 3.48 (2H, s), 6.76 (1H, s), 7.21-7.33 (6H, m), 7.44 (1H, s)

MS (ESI+, m/e) 257 (M+1)

### Reference Example 152

### (3R)-1-Benzyl-3-(4-bromobenzyl)piperazine

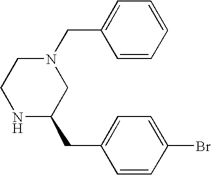

¹H-NMR (CDCl₃) δ 1.62 (1H, br s), 1.87 (1H, t), 2.07 (1H, dt), 2.50 (1H, dd), 2.66 (1H, dd), 2.71-2.99 (5H, m), 3.46 (1H, d), 3.53 (1H, d), 7.07 (2H, d), 7.21-7.32 (5H, m), 7.41 (2H, d)

MS (ESI+, m/e) 345 (M+1)

### Reference Example 153

### (3R)-1-Benzyl-3-(pyridin-3-ylmethyl)piperazine

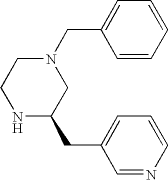

(3R)-1-Benzyl-3-(pyridin-3-ylmethyl)piperazine-2,5-dione (4.13 g) was dissolved in THF (60 ml), and borane-THF (1.0M THF solution, 111.9 ml) was added dropwise at room temperature over 15 min. The mixture was stirred at room temperature for 1 hr, and then at 60° C. for 15 hr. The reaction mixture was ice-cooled, water (6.5 ml) was added dropwise over 5 min, and the mixture was stirred at room temperature for 10 min. The reaction mixture was concentrated under reduced pressure, to the residue was added 2N hydrochloric acid (65 ml), and the mixture was stirred at 50° C. for 30 min. The reaction mixture was ice-cooled again, basified with 8N aqueous sodium hydroxide solution to weak basic (pH 8-9), and extracted with ethyl acetate. The extract was washed with saturated brine, and dried over anhydrous magnesium sulfate, and the solvent was evaporated under reduced pressure. The residue was subjected to silica gel column chromatography, and the fraction eluted with ethyl acetate-methanol-triethylamine (1:0:0-100:5:2) was concentrated under reduced pressure to give the object compound (1.98 g) as an oil.

¹H-NMR (CDCl₃) δ 1.76 (1H, br s), 1.88 (1H, t), 2.08 (1H, dt), 2.57 (1H, dd), 2.67-3.04 (6H, m), 3.46 (1H, d), 3.53 (1H, d), 7.19-7.34 (6H, m), 7.50 (1H, dt), 8.45-8.47 (2H, m)

MS (ESI+, m/e) 268 (M+1)

### Reference Example 154

### (2R)-2-(2,4-Dichlorobenzyl)piperazine

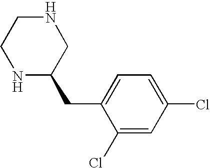

(3R)-3-(2,4-Dichlorobenzyl)piperazine-2,5-dione (3.39 g) was dissolved in THF (55 ml), and borane-THF (1.0M THF solution, 99.3 ml) was added dropwise at room temperature over 15 min. The mixture was stirred at room temperature for 1 hr, and then at 60° C. for 6 hr, and the reaction mixture was ice-cooled. Water (6 ml) was added dropwise over 5 min, and the mixture was stirred at room temperature for 10 min, and concentrated under reduced pressure. To the residue was added 2N hydrochloric acid (60 ml), and the mixture was stirred at 50° C. for 30 min. The reaction mixture was ice-cooled again, basified with 8N aqueous sodium hydroxide solution to weak basic (pH 8-9), and extracted with chloroform. The extract was washed with saturated brine, and dried over anhydrous magnesium sulfate. The solvent was evaporated under reduced pressure, and the crystals were collected by filtration to give the object compound (1.09 g).

MS (ESI+, m/e) 245 (M+1)

### Reference Example 155

### tert-Butyl (3R)-3-(2,4-dichlorobenzyl)piperazine-1-carboxylate

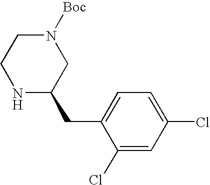

A mixture of (2R)-2-(2,4-dichlorobenzyl)piperazine (1.08 g), tert-butanol (20 ml), water (15 ml) and 1N aqueous sodium hydroxide solution (4.63 ml) was ice-cooled, and di-tert-butyl bicarbonate (1.01 g) was added thereto. The mixture was stirred at room temperature for 6 hr, and concentrated under reduced pressure to about half-volume. The residue was saturated with sodium chloride, and extracted with ethyl acetate. The extract was washed with saturated brine, and dried over anhydrous magnesium sulfate, and the solvent was evaporated under reduced pressure. The residue was subjected to silica gel column chromatography, and the fraction eluted with ethyl acetate-hexane-methanol (1:8:0-20:0:1) was concentrated under reduced pressure to give the object compound (154 mg) as an oil.

¹H-NMR (CDCl₃) δ 1.45 (9H, s), 2.62-2.71 (3H, m), 2.81-2.95 (5H, m), 3.87-3.91 (2H, m), 7.15-7.21 (2H, m), 7.38 (1H, s)

MS (ESI+, m/e) 345 (M+1)

### Reference Example 156

### tert-Butyl (3R)-3-(1,3-thiazol-4-ylmethyl)piperazine-1-carboxylate

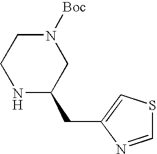

A mixture of (3R)-3-(1,3-thiazol-4-ylmethyl)piperazine-2,5-dione (1.0 g) and THF (30 ml) was ice-cooled, and lithium aluminum hydride (0.9 g) was added by small portions. The mixture was stirred at room temperature for 30 min, and then at 50° C. for 2 hr, and cooled to −78° C. Sodium sulfate hydrate and 1N aqueous sodium hydroxide solution (0.5 ml) were successively added dropwise. After the completion of the dropwise addition, the mixture was stirred at room temperature for 1 hr. The insoluble material was filtered, and washed with ethyl acetate. The filtrate was washed with saturated brine, and dried over anhydrous magnesium sulfate, and the solvent was evaporated under reduced pressure. The residue was subjected to basic silica gel column chromatography, and the fraction eluted with ethyl acetate-methanol (1:1) was concentrated under reduced pressure. The residue was dissolved in tert-butanol (5 ml), 2.5N aqueous sodium hydroxide solution (5 ml) and di-tert-butyl bicarbonate (2.18 g) were successively added, and the mixture was stirred at room temperature for 2 hr. The solvent was evaporated under reduced pressure, and the residue was dissolved in ethyl acetate (50 ml). The solution was washed successively with water and saturated brine, and dried over anhydrous sodium sulfate, and the solvent was evaporated under reduced pressure. The residue was subjected to basic silica gel column chromatography, and the fraction eluted with ethyl acetate-methanol (4:1) was concentrated under reduced pressure to give the object compound (100 mg) as an oil.

MS (ESI+, m/e) 284 (M+1)

### Reference Example 157

### tert-Butyl (2R)-4-benzyl-2-(4-bromobenzyl)piperazine-1-carboxylate

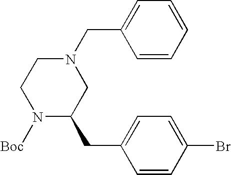

(3R)-1-Benzyl-3-(4-bromobenzyl)piperazine (2.64 g) was dissolved in THF (20 ml), and di-tert-butyl bicarbonate (1.75 g) was added. The mixture was stirred at room temperature for 3 hr, and the reaction mixture was concentrated under reduced pressure. The crystals were collected by filtration to give the object compound (2.87 g).

¹H-NMR (CDCl₃) δ 1.38 (9H, s), 1.95 (1H, dd), 2.08 (1H, dt), 2.58 (1H, d), 2.82-2.98 (3H, m), 3.17 (1H, dt), 3.31 (1H, d), 3.55 (1H, d), 3.91-4.08 (2H, m), 6.87 (2H, d), 7.24-7.34 (7H, m)

MS (ESI+, m/e) 445 (M+1)

### Reference Example 158

### tert-Butyl (2R)-4-benzyl-2-(4-morpholinobenzyl)piperazine-1-carboxylate

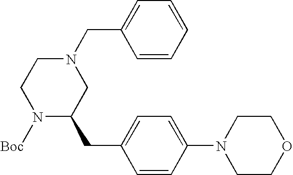

A mixture of tert-butyl (2R)-4-benzyl-2-(4-bromobenzyl)piperazine-1-carboxylate (1.00 g), morpholine (215 mg), BINAP (140 mg), sodium tert-butoxide (324 mg), Pd₂(dba)₃ (82 mg) and toluene (20 ml) was stirred at 90° C. for 15 hr in an argon stream, and poured into saturated aqueous sodium hydrogen carbonate, and the mixture was extracted with ethyl acetate. The extract was washed successively with water and saturated brine, and dried over anhydrous magnesium sulfate, and the solvent was evaporated under reduced pressure. The residue was subjected to silica gel column chromatography, and the fraction eluted with ethyl acetate-hexane (1:5-1:2) was concentrated under reduced pressure to give the object compound (986 mg) as an oil (it was allowed to crystallization at low temperature).

¹H-NMR (CDCl₃) δ 1.39 (9H, s), 1.95 (1H, dd), 2.04 (1H, dt), 2.63 (1H, d), 2.72-2.84 (2H, m), 2.96-3.04 (1H, m), 3.07 (4H, dd), 3.18 (1H, dt), 3.36 (1H, d), 3.51 (1H, d), 3.84 (4H, dd), 3.85-4.15 (2H, m), 6.72 (2H, d), 6.95 (2H, d), 7.27-7.34 (5H, m)

MS (ESI+, m/e) 452 (M+1)

### Reference Example 159

### 4-(4-{[(2R)-4-Benzylpiperazin-2-yl]methyl}phenyl)morpholine

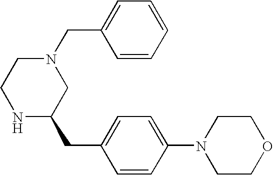

tert-Butyl (2R)-4-benzyl-2-(4-morpholinobenzyl)piperazine-1-carboxylate (937 mg) was dissolved in dichloromethane (2 ml). TFA (5 ml) was added, and the mixture was stirred at room temperature for 50 min. The reaction mixture was poured into saturated aqueous sodium hydrogen carbonate by small portions, and the mixture was saturated with sodium chloride, and extracted with ethyl acetate. The extract was washed with saturated brine, and dried over anhydrous magnesium sulfate, and the solvent was evaporated under reduced pressure. The residue was subjected to basic silica gel column chromatography, and the fraction eluted with ethyl acetate-hexane (1:2-1:0) was concentrated under reduced pressure to give the object compound (728 mg) as an oil.

¹H-NMR (CDCl₃) δ 1.63 (1H, br s), 1.87 (1H, t), 2.07 (1H, dt), 2.45 (1H, dd), 2.63 (1H, dd), 2.71-2.97 (5H, m), 3.13 (4H, dd), 3.46 (1H, d), 3.53 (1H, d), 3.84 (4H, dd), 6.84 (2H, d), 7.09 (2H, d), 7.21-7.31 (5H, m)

MS (ESI+, m/e) 352 (M+1)

### Reference Example 160

### Di-tert-butyl (2R)-2-(4-hydroxybenzyl)piperazine-1,4-dicarboxylate

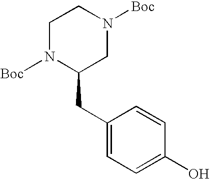

4-{[(2R)-4-Benzylpiperazin-2-yl]methyl}phenol (12 g) was dissolved in methanol (240 ml), 20% palladium hydroxide-carbon (50% containing water, 3.0 g) was added thereto, and the mixture was subjected to catalytic reduction at ambient temperature and normal pressure for 12 hr. The catalyst was filtered off, and the filtrate was concentrated under reduced pressure. The residue was suspended in ethyl acetate, and the suspension was dried over anhydrous sodium sulfate, and the solvent was evaporated under reduced pressure. The residue was dissolved in a mixed solvent of tert-butanol (100 ml) and water (100 ml), and 2.5N sodium hydroxide (40 ml) and di-tert-butyl bicarbonate (17.6 g) were added under ice-cooling. After stirring for 12 hr, the mixture was extracted with ethyl acetate. The extract was washed successively with 10% aqueous citric acid solution and saturated brine, and dried over anhydrous sodium sulfate, and the solvent was evaporated under reduced pressure. The residue was subjected to silica gel column chromatography, and the fraction eluted with ethyl acetate-hexane (1:4) was concentrated under reduced pressure to give the object compound (10.7 g) as an amorphous solid.

MS (ESI+, m/e) 393 (M+1)

### Reference Example 161

### Di-tert-butyl (2R)-2-(4-{[(trifluoromethyl)sulfonyl]oxy}benzyl)piperazine-1,4-dicarboxylate

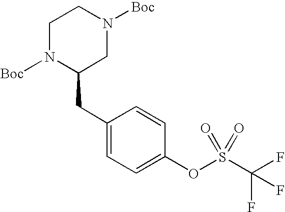

Di-tert-butyl (2R)-2-(4-hydroxybenzyl)piperazine-1,4-dicarboxylate (10.7 g), 4-nitrophenyl trifluoromethanesulfonate (8.1 g) and potassium carbonate (7.6 g) were suspended in DMF (170 ml), and the suspension was stirred at room temperature for 12 hr. The reaction mixture was poured into water, and the mixture was extracted with ethyl acetate. The extract was washed with saturated brine, and dried over anhydrous sodium sulfate, and the solvent was evaporated under reduced pressure. The residue was subjected to silica gel column chromatography, and the fraction eluted with ethyl acetate-hexane (1:1) was concentrated under reduced pressure to give the object compound (11.2 g) as an amorphous solid.

MS (ESI+, m/e) 525 (M+1)

### Reference Example 162

### tert-Butyl (3R)-3-(4-cyanobenzyl)piperazine-1-carboxylate

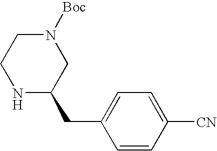

A solution of di-tert-butyl (2R)-2-(4-{[(trifluoromethyl)sulfonyl]oxy}benzyl)piperazine-1,4-dicarboxylate (1.05 g), zinc cyanide (282 mg) and tetrakis(triphenylphosphine)palladium(0) (231 mg) in DMF (10 ml) was stirred at 80° C. for 15 hr. The insoluble material was filtered off, and the solvent was evaporated under reduced pressure. The residue was subjected to silica gel column chromatography, and the fraction eluted with ethyl acetate-hexane (1:9-3:7) was concentrated under reduced pressure to give di-tert-butyl (2R)-2-(4-cyanobenzyl)piperazine-1,4-dicarboxylate (570 mg) as crystals.

The total amount thereof was dissolved in dichloromethane (1 ml), and TFA (3 ml) was added thereto. The mixture was stirred at room temperature for 1 hr, and concentrated under reduced pressure. To the residue was 6% aqueous sodium bicarbonate was added by small portions to neutralize the residue, and the mixture was extracted with chloroform. The extract was dried over anhydrous magnesium sulfate, and the solvent was evaporated under reduced pressure to give 4-[(2R)-piperazin-2-ylmethyl]benzonitrile (600 mg) as an oil.

The total amount thereof and aqueous sodium hydroxide solution (100 mg/10 ml) were dissolved in tert-butanol (10 ml), and the solution was ice-cooled. Di-tert-butyl bicarbonate (546 mg) was added thereto, and the mixture was stirred at room temperature for 15 hr. The reaction mixture was concentrated under reduced pressure. The residue was subjected to silica gel column chromatography, and the fraction eluted with ethyl acetate-methanol (1:0-4:1) was concentrated under reduced pressure to give the object compound (145 mg) as an amorphous solid.

MS (ESI+, m/e) 302 (M+1)

### Reference Example 163

### tert-Butyl (3S)-4-benzyl-3-(hydroxymethyl)piperazine-1-carboxylate

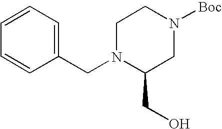

tert-Butyl (3S)-3-(hydroxymethyl)piperazine-1-carboxylate (15.1 g), benzaldehyde (7.4 g) and acetic acid (4.2 g) were dissolved in 1,2-dichloroethane (200 ml), and the solution was ice-cooled. Sodium triacetoxyborohydride (19.3 g) was added, and the mixture was stirred at room temperature for 15 hr, and neutralized with saturated aqueous sodium hydrogen carbonate. The organic layer was dried over anhydrous magnesium sulfate, and concentrated under reduced pressure. The residue was subjected to silica gel column chromatography, and the fraction eluted with ethyl acetate-hexane (1:4-1:1) was concentrated under reduced pressure to give the object compound (16.1 g) as crystals.

MS (ESI+, m/e) 307 (M+1)

### Reference Example 164

### tert-Butyl (2S)-4-benzyl-2-(hydroxymethyl)piperazine-1-carboxylate

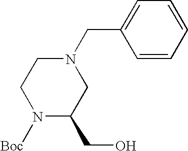

[(2S)-4-Benzylpiperazin-2-yl]methanol (25.84 g) was dissolved in THF (250 ml). Di-tert-butyl bicarbonate (27.34 g) was added by small portions, and the mixture was stirred at room temperature for 2 hr, and concentrated under reduced pressure. The residue was subjected to silica gel column chromatography, and the fraction eluted with ethyl acetate-hexane (1.5:1) was concentrated under reduced pressure to give the object compound (38.34 g) as an oil.

¹H-NMR (CDCl₃) δ 1.45 (9H, s), 2.09 (1H, dt), 2.31 (1H, dd), 2.83 (1H, d), 2.97 (1H, d), 3.36-3.53 (3H, m), 3.83-3.99 (5H, m), 7.25-7.33 (5H, m)

MS (ESI+, m/e) 307 (M+1)

In the same manner as in Reference Example 164, the following compound (Reference Example 165) was obtained.

### Reference Example 165

### tert-Butyl (2R)-4-benzyl-2-(2-hydroxyethyl)piperazine-1-carboxylate

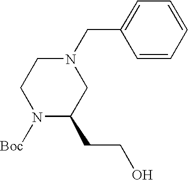

¹H-NMR (CDCl₃) δ 1.46 (9H, s), 2.01 (1H, dt), 2.20-2.24 (1H, m), 2.25 (1H, dd), 2.68-2.72 (2H, m), 3.01 (1H, dt), 3.37-3.60 (4H, m), 3.85-3.98 (3H, m), 4.26-4.30 (1H, m), 7.25-7.34 (5H, m)

MS (ESI+, m/e) 321 (M+1)

### Reference Example 166

### tert-Butyl (2R)-4-benzyl-2-(3-hydroxypropyl)piperazine-1-carboxylate

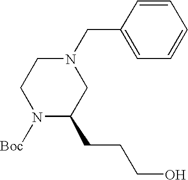

Benzyl 3-[(2R)-4-benzyl-3,6-dioxopiperazin-2-yl]propionate (25 g) was dissolved in THF (350 ml), and the solution was cooled to −20° C. Lithium aluminum hydride (13 g) was added over 30 min, and the mixture was stirred at room temperature for 30 min, and then at 50° C. for 12 hr. The mixture was cooled to −78° C., and sodium sulfate hydrate and 1N aqueous sodium hydroxide solution (5 ml) were successively added dropwise. After the completion of the dropwise addition, the mixture was stirred at room temperature for 1 hr. The insoluble material was filtered, and washed with ethyl acetate. The filtrate was washed with saturated brine, and dried over anhydrous sodium sulfate, and the solvent was evaporated under reduced pressure. The residue was dissolved in THF (150 ml), di-tert-butyl bicarbonate (13.4 g) was added, and the mixture was stirred at room temperature for 2 hr. The solvent was evaporated under reduced pressure, and the residue was subjected to silica gel column chromatography. The fraction eluted with ethyl acetate-methanol (1:1) was concentrated under reduced pressure to give the object compound (8.2 g) as an oil.

MS (ESI+, m/e) 335 (M+1)

### Reference Example 167

### tert-Butyl (2S)-4-benzyl-2-formylpiperazine-1-carboxylate

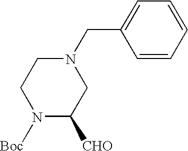

tert-Butyl (2S)-4-benzyl-2-(hydroxymethyl)piperazine-1-carboxylate (12.26 g) was dissolved in dichloromethane (130 ml), and a solution of pyridine-sulfur trioxide complex (19.10 g) in DMSO (130 ml) and triethylamine (12.14 g) were added at 0° C. The reaction mixture was stirred at 0° C. for 2 hr, and poured into ice-cooled saturated aqueous sodium hydrogen carbonate, and the mixture was extracted with ethyl acetate. The extract was washed successively with water and saturated brine, and dried over anhydrous magnesium sulfate, and the solvent was evaporated under reduced pressure. The residue was subjected to silica gel column chromatography, and the fraction eluted with ethyl acetate-hexane (1:4) was concentrated under reduced pressure to give the object compound (6.28 g) as an oil.

¹H-NMR (CDCl₃) δ 1.43-1.48 (9H, m), 2.12 (1H, dt), 2.27 (1H, dd), 2.69-2.73 (1H, m), 3.06-3.15 (1H, m), 3.30 (1H, d), 3.44 (1H, d), 3.56 (1H, d), 3.78 (0.5H, d), 3.90 (0.5H, d), 4.38 (0.5H, s), 4.58 (0.5H, s), 7.22-7.34 (5H, m), 9.49 (1H, s)

MS (ESI+, m/e) 305 (M+1)

In the same manner as in Reference Example 167, the following compound (Reference Example 168) was obtained.

### Reference Example 168

### tert-Butyl (2R)-4-benzyl-2-(2-oxoethyl)piperazine-1-carboxylate

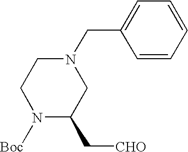

¹H-NMR (CDCl₃) δ 1.44 (9H, s), 2.04 (1H, dt), 2.20 (1H, dd), 2.66-2.84 (4H, m), 3.01-3.09 (1H, m), 3.42 (1H, d), 3.51 (1H, d), 3.84-3.88 (1H, m), 4.60-4.64 (1H, m), 7.25-7.28 (5H, m), 9.73 (1H, s)

MS (ESI+, m/e) 319 (M+1)

### Reference Example 169

### tert-Butyl (3S)-4-benzyl-3-(bromomethyl)piperazine-1-carboxylate

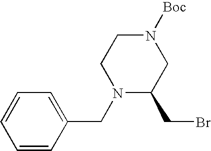

Triphenylphosphine (9.4 g) and carbon tetrabromide (11.9 g) were suspended in diethyl ether (200 ml), tert-butyl (3S)-4-benzyl-3-(hydroxymethyl)piperazine-1-carboxylate (9.2 g) was added over 5 min by small portions, and the mixture was stirred at room temperature for 15 hr. The insoluble material was filtered off, and the filtrate was concentrated under reduced pressure. The residue was subjected to silica gel column chromatography, and the fraction eluted with ethyl acetate-hexane (1:9-3:7) was concentrated under reduced pressure to give the object compound (8.5 g) as an oil.

MS (ESI+, m/e) 370 (M+1)

### Reference Example 170

### tert-Butyl (3S)-3-[(phenylthio)methyl]piperazine-1-carboxylate

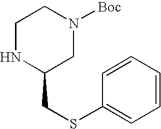

tert-Butyl (3S)-4-benzyl-3-(bromomethyl)piperazine-1-carboxylate (3.69 g) was dissolved in DMF (35 ml). Sodium benzenethiolate (1.98 g) was added, and the mixture was stirred at room temperature for 15 hr. To the reaction mixture was added 6% aqueous sodium bicarbonate, and the mixture was extracted with ethyl acetate. The extract was dried over anhydrous magnesium sulfate, and concentrated under reduced pressure. The residue was subjected to silica gel column chromatography, and the fraction eluted with ethyl acetate-hexane (1:9-3:7) was concentrated under reduced pressure to give tert-butyl (3S)-4-benzyl-3-[(phenylthio)methyl]piperazine-1-carboxylate (3.77 g) as an oil. 3.67 g therefrom was dissolved in 1,2-dichloroethane (30 ml), and 1-chloroethyl chloroformate (1.58 g) was added. The mixture was heated under reflux for 5 hr, and concentrated under reduced pressure. To the residue was added methanol (30 ml), and the mixture was further heated under reflux for 4 hr, and concentrated under reduced pressure. The residue was neutralized with 6% aqueous sodium bicarbonate, and the mixture was extracted with ethyl acetate. The extract was washed with saturated brine, dried over anhydrous magnesium sulfate, and concentrated under reduced pressure. The residue was subjected to silica gel column chromatography, and the fraction eluted with ethyl acetate-methanol (1:0-9:1) was concentrated under reduced pressure to give the object compound (1.44 g) as an oil.

MS (ESI+, m/e) 309 (M+1)

### Reference Example 171

### tert-Butyl (2S)-4-benzyl-2-{2,2,2-trifluoro-1-[(trimethylsilyl)oxy]ethyl}piperazine-1-carboxylate

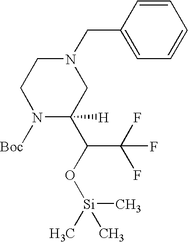

tert-Butyl (2S)-4-benzyl-2-formylpiperazine-1-carboxylate (912 mg) and trimethyl(trifluoromethyl)silane (853 mg) were dissolved in THF (20 ml). TBAF (several mg) was added, and the mixture was stirred at room temperature for 4 hr, and concentrated under reduced pressure to give the object compound (1.34 g) as an oil.

MS (ESI+, m/e) 447 (M+1)

### Reference Example 172

### tert-Butyl (2S)-4-benzyl-2-[cyclopropyl(hydroxy)methyl]piperazine-1-carboxylate

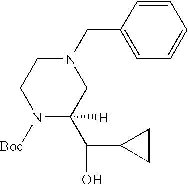

tert-Butyl (2S)-4-benzyl-2-formylpiperazine-1-carboxylate (2.5 g) was dissolved in THF (25 ml), and the mixture was cooled to −30° C. Cyclopropylmagnesium bromide (0.5M THF solution, 40 ml) was added thereto, and the mixture was stirred at −20° C. for 1 hr. To the reaction mixture was added saturated aqueous ammonium chloride solution, and the mixture was extracted with ethyl acetate. The extract was washed with saturated brine, dried over anhydrous sodium sulfate, and concentrated under reduced pressure. The residue was subjected to basic silica gel column chromatography, and the fraction eluted with ethyl acetate was concentrated under reduced pressure to give the object compound (2.2 g) as an amorphous solid.

MS (ESI+, m/e) 347 (M+1)

In the same manner as in Reference Example 172 and by the reaction of known methyl (1,4-dibenzylpiperazin-2-yl)acetate with methylmagnesium bromide, the following compound (Reference Example 173) was obtained.

### Reference Example 173

### 1-(1,4-Dibenzylpiperazin-2-yl)-2-methylpropan-2-ol

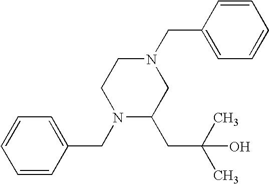

MS (ESI+, m/e) 339 (M+1)

### Reference Example 174

### 1-[(2S)-4-Benzylpiperazin-2-yl]-2,2,2-trifluoroethanol

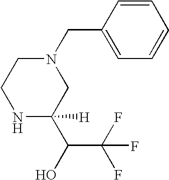

tert-Butyl (2S)-4-benzyl-2-{2,2,2-trifluoro-1-[(trimethylsilyl)oxy]ethyl}piperazine-1-carboxylate (1.34 g) was dissolved in chloroform (2 ml). TFA (2 ml) was added, and the mixture was stirred at room temperature for 2 hr. The reaction mixture was poured into saturated aqueous sodium hydrogen carbonate by small portions, and the mixture was basified with small amount of potassium carbonate, and extracted with ethyl acetate. The extract was washed with saturated brine, and dried over anhydrous magnesium sulfate, and the solvent was evaporated under reduced pressure. The residue was subjected to basic silica gel column chromatography, and the fraction eluted with ethyl acetate was concentrated under reduced pressure to give the object compound (772 mg) as an oil.

MS (ESI+, m/e) 275 (M+1)

In the same manner as in Reference Example 174, the following compound (Reference Example 175) was obtained.

### Reference Example 175

### [(2S)-4-Benzylpiperazin-2-yl](cyclopropyl)methanol

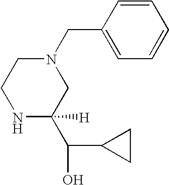

MS (ESI+, m/e) 247 (M+1)

### Reference Example 176

### tert-Butyl 3-(2-hydroxy-2-methylpropyl)piperazine-1-carboxylate

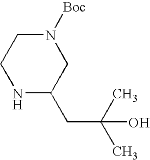

1-(1,4-Dibenzylpiperazin-2-yl)-2-methylpropan-2-ol (1.0 g) was dissolved in methanol (30 ml), 20% palladium hydroxide-carbon (50% containing water, 200 mg) was added thereto, and the mixture was subjected to catalytic reduction at ambient temperature and normal pressure for 17 hr. The catalyst was filtered off, and the filtrate was concentrated under reduced pressure. The residue and potassium carbonate (300 mg) were dissolved in THF (15 ml) and water (30 ml), and the mixture was cooled to 0° C. (2Z)-{[(tert-Butoxycarbonyl)oxy]imino}(phenyl)acetonitrile (726 mg) was added thereto, and the mixture was stirred at the same temperature for 1 hr, and then at room temperature for 3 hr. To the reaction mixture was added 30% aqueous citric acid solution, and the mixture was washed with diethyl ether twice. The aqueous layer was saturated with potassium carbonate, and extracted with ethyl acetate. The extract was washed with saturated brine, dried over anhydrous magnesium sulfate, and concentrated under reduced pressure to give the object compound (500 mg) as an oil.

MS (ESI+, m/e) 259 (M+1)

### Reference Example 177

### tert-Butyl (2R)-4-benzyl-2-[(isopropylamino)methyl]piperazine-1-carboxylate

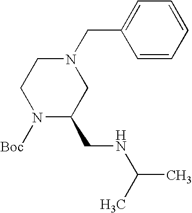

A solution of tert-butyl (2S)-4-benzyl-2-formylpiperazine-1-carboxylate (6.27 g), isopropylamine (2.44 g), acetic acid (2.47 g) and dichloromethane (80 ml) in DMF (40 ml) was stirred at room temperature for 40 min, sodium triacetoxyborohydride (8.73 g) was added, and the mixture was further stirred at room temperature for 15 hr. The reaction mixture was poured into saturated aqueous sodium hydrogen carbonate, and the mixture was stirred at room temperature for 15 min. After stirring, the mixture was extracted with ethyl acetate. The extract was washed successively with water and saturated brine, and dried over anhydrous magnesium sulfate, and the solvent was evaporated under reduced pressure to give the object compound (6.37 g) as an oil.

¹H-NMR (CDCl₃) δ 0.98 (3H, d), 1.00 (3H, d), 1.46 (9H, s), 1.99-2.08 (2H, m), 2.73-2.96 (6H, m), 3.07 (1H, dt), 3.38 (1H, d), 3.54 (1H, d), 3.85-3.89 (1H, m), 4.07 (1H, br s), 7.30-7.32 (5H, m)

MS (ESI+, m/e) 348 (M+1)

In the same manner as in Reference Example 177, the following compounds (Reference Examples 178-179) were obtained.

### Reference Example 178

### tert-Butyl (2R)-2-(anilinomethyl)-4-benzylpiperazine-1-carboxylate

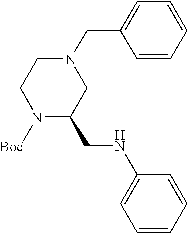

¹H-NMR (CDCl₃) δ 1.45 (9H, s), 2.02-2.11 (2H, m), 2.80-2.84 (2H, m), 3.12 (1H, dt), 3.39-4.28 (7H, m), 6.54 (2H, d), 6.62-6.67 (1H, m), 7.10-7.15 (2H, m), 7.27-7.34 (5H, m)

MS (ESI+, m/e) 382 (M+1)

### Reference Example 179

### tert-Butyl (2R)-4-benzyl-2-{[(2,4-dimethoxybenzyl)amino]methyl}piperazine-1-carboxylate

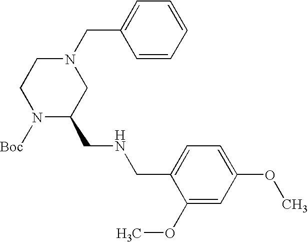

¹H-NMR (CDCl₃) δ 1.44 (9H, s), 1.59 (1H, br s), 1.97 (1H, dd), 2.00 (1H, dd), 2.09 (1H, dd), 2.71 (1H, d), 2.85-3.03 (4H, m), 3.46 (2H, s), 3.71 (2H, s), 3.77 (3H, s), 3.80 (3H, s), 3.80-3.86 (1H, m), 6.40-6.46 (2H, m), 7.12 (1H, d), 7.20-7.33 (5H, m)

MS (ESI+, m/e) 456 (M+1)

### Reference Example 180

### tert-Butyl (2S)-4-benzyl-2-{[(4-ethoxy-4-oxobutanoyl)(phenyl)amino]methyl}piperazine-1-carboxylate

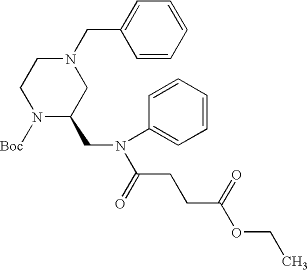

tert-Butyl (2R)-2-(anilinomethyl)-4-benzylpiperazine-1-carboxylate (2.75 g) and triethylamine (1.46 g) were dissolved in THF (60 ml), ethylsuccinyl chloride (2.37 g) was added, and the mixture was stirred at room temperature for 3 hr. The mixture was poured into saturated aqueous sodium hydrogen carbonate, and extracted with ethyl acetate. The extract was washed successively with water and saturated brine, and dried over anhydrous magnesium sulfate, and the solvent was evaporated under reduced pressure. The residue was subjected to basic silica gel column chromatography, and the fraction eluted with ethyl acetate-chloroform-hexane (1:1:4) was concentrated under reduced pressure to give the object compound (3.56 g) as an oil.

MS (ESI+, m/e) 510 (M+1)

### Reference Example 181

### tert-Butyl (2S)-4-benzyl-2-{[(2,4-dimethoxybenzyl)(2-methoxybenzoyl)amino]methyl}piperazine-1-carboxylate

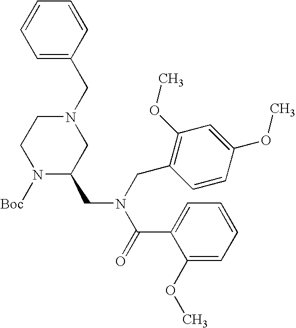

tert-Butyl (2R)-4-benzyl-2-{[(2,4-dimethoxybenzyl)amino]methyl}piperazine-1-carboxylate (1.91 g) and triethylamine (850 mg) were dissolved in THF (35 ml), and 2-methoxybenzoyl chloride (1.43 g) was added. The mixture was stirred at room temperature for 15 hr, and poured into saturated aqueous sodium hydrogen carbonate, and the mixture was extracted with ethyl acetate. The extract was washed successively with water and saturated brine, and dried over anhydrous magnesium sulfate, and the solvent was evaporated under reduced pressure. The residue was subjected to silica gel column chromatography, and the fraction eluted with ethyl acetate-hexane (1:2-1:1) was concentrated under reduced pressure to give the object compound (1.90 g) as an amorphous solid.

MS (ESI+, m/e) 590 (M+1)

In the same manner as in Reference Example 181, the following compounds (Reference Examples 182-184) were obtained.

### Reference Example 182

### tert-Butyl (2S)-2-{[(benzoyl)(2,4-dimethoxybenzyl)amino]methyl}-4-benzylpiperazine-1-carboxylate

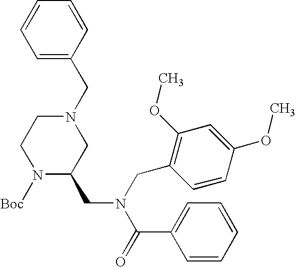

MS (ESI+, m/e) 560 (M+1)

### Reference Example 183

### tert-Butyl (2S)-4-benzyl-2-{[(3,5-difluorobenzoyl)(2,4-dimethoxybenzyl)amino]methyl}piperazine-1-carboxylate

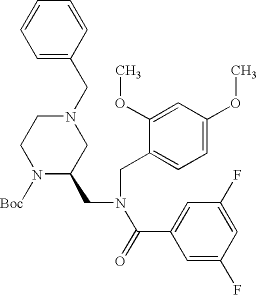

MS (ESI+, m/e) 596 (M+1)

### Reference Example 184

### tert-Butyl (2S)-4-benzyl-2-{[(cyclohexylcarbonyl)(2,4-dimethoxybenzyl)amino]methyl}piperazine-1-carboxylate

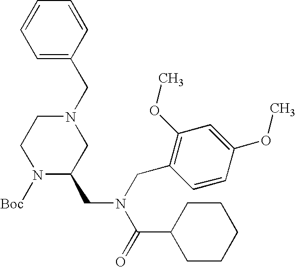

MS (ESI+, m/e) 566 (M+1)

### Reference Example 185

### tert-Butyl (2S)-4-benzyl-2-{[(isopropyl)(5-methoxy-4,4-dimethyl-5-oxopentanoyl)amino]methyl}piperazine-1-carboxylate

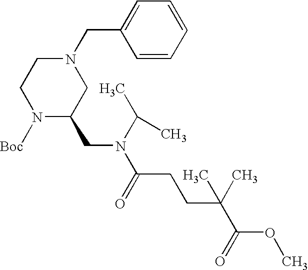

5-Methoxy-4,4-dimethyl-5-oxovaleric acid (4.46 g) was dissolved in THF (100 ml), and oxalyl chloride (3.90 g) and DMF (50 μl) were added. The mixture was stirred at room temperature for 2 hr, and concentrated under reduced pressure. The residue was dissolved in THF (10 ml), and the solution was added to a solution of tert-butyl (2R)-4-benzyl-2-[(isopropylamino)methyl]piperazine-1-carboxylate (4.24 g) and triethylamine (2.59 g) in THF (90 ml). The mixture was stirred at room temperature for 15 hr, and poured into saturated aqueous sodium hydrogen carbonate, and the mixture was extracted with ethyl acetate. The extract was washed successively with water and saturated brine, and dried over anhydrous magnesium sulfate, and the solvent was evaporated under reduced pressure. The residue was subjected to silica gel column chromatography, and the fraction eluted with ethyl acetate-hexane (1:3-1:1) was concentrated under reduced pressure to give the object compound (5.91 g) as an oil.

MS (ESI+, m/e) 504 (M+1)

In the same manner as in Reference Example 185, the following compound (Reference Example 186) was obtained.

### Reference Example 186

### tert-Butyl (2S)-4-benzyl-2-{[(5-methoxy-4,4-dimethyl-5-oxopentanoyl)(phenyl)amino]methyl}piperazine-1-carboxylate

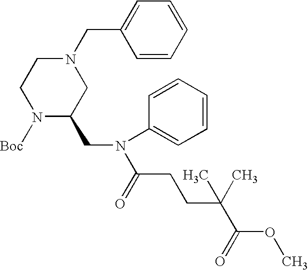

MS (ESI+, m/e) 538 (M+1)

### Reference Example 187

### 4-[{[(2S)-4-Benzyl-1-(tert-butoxycarbonyl)piperazin-2-yl]methyl}(phenyl)amino]-4-oxobutyric acid

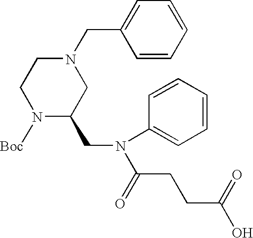

tert-Butyl (2S)-4-benzyl-2-{[(4-ethoxy-4-oxobutanoyl)(phenyl)amino]methyl}piperazine-1-carboxylate (3.55 g) was dissolved in ethanol (115 ml), and 2N aqueous lithium hydroxide solution (75 ml) was added. The mixture was stirred at room temperature for 1 hr, and poured into ice water. While vigorously stirring the mixture, 6N hydrochloric acid was added by small portions to neutralize the mixture. The mixture was saturated with sodium chloride, and extracted with ethyl acetate. The extract was washed with saturated brine, and dried over anhydrous magnesium sulfate. The solvent was evaporated under reduced pressure to give the object compound (3.21 g) as an amorphous solid.

MS (ESI+, m/e) 482 (M+1)

### Reference Example 188

### tert-Butyl (2S)-2-{[(4-amino-4-oxobutanoyl)(phenyl)amino]methyl}-4-benzylpiperazine-1-carboxylate

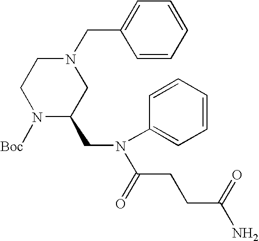

A mixture of 4-[{[(2S)-4-benzyl-1-(tert-butoxycarbonyl)piperazin-2-yl]methyl}(phenyl)amino]-4-oxobutyric acid (3.20 g), HOBt ammonium salt (1.21 g), WSC.HCl (1.53 g) and DMF (45 ml) was stirred at room temperature for 15 hr, and poured into saturated aqueous sodium hydrogen carbonate, and the mixture was extracted with ethyl acetate. The extract was washed successively with water and saturated brine, and dried over anhydrous magnesium sulfate, and the solvent was evaporated under reduced pressure to give the object compound (3.00 g) as an amorphous solid.

MS (ESI+, m/e) 481 (M+1)

### Reference Example 189

### Methyl 5-[{[(2S)-4-benzylpiperazin-2-yl]methyl}(isopropyl)amino]-2,2-dimethyl-5-oxovalerate

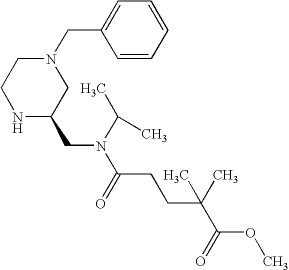

tert-Butyl (2S)-4-benzyl-2-{[(isopropyl)(5-methoxy-4,4-dimethyl-5-oxopentanoyl)amino]methyl}piperazine-1-carboxylate (3.03 g) was dissolved in dichloromethane (7.5 ml), TFA (15 ml) was added, and the mixture was stirred at room temperature for 1 hr. The reaction mixture was poured into saturated aqueous sodium hydrogen carbonate by small portions, and potassium carbonate was added by small portions to basify the mixture. The mixture was saturated with sodium chloride, and extracted with ethyl acetate. The extract was washed with saturated brine, and dried over anhydrous magnesium sulfate. The solvent was evaporated under reduced pressure to give the object compound (2.41 g) as an oil.

MS (ESI+, m/e) 404 (M+1)

In the same manner as in Reference Example 189, the following compounds (Reference Examples 190-191) were obtained.

### Reference Example 190

### Methyl 5-[{[(2S)-4-benzylpiperazin-2-yl]methyl}(phenyl)amino]-2,2-dimethyl-5-oxovalerate

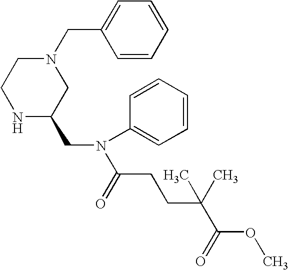

MS (ESI+, m/e) 438 (M+1)

### Reference Example 191

### N-{[(2S)-4-Benzylpiperazin-2-yl]methyl}-N-phenylsuccinamide

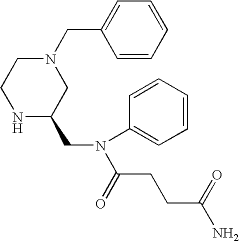

MS (ESI+, m/e) 381 (M+1)

### Reference Example 192

### N-{[(2R)-4-Benzylpiperazin-2-yl]methyl}-2-methoxybenzamide

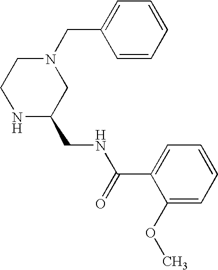

tert-Butyl (2S)-4-benzyl-2-{[(2,4-dimethoxybenzyl)(2-methoxybenzoyl)amino]methyl}piperazine-1-carboxylate (1.89 g) was dissolved in dichloromethane (3 ml), TFA (12 ml) was added, and the mixture was stirred at room temperature for 1.5 hr. The reaction mixture was poured into saturated aqueous sodium hydrogen carbonate by small portions. To the mixture was added potassium carbonate by small portions to basify the mixture, and the mixture was extracted with ethyl acetate (along with which the insoluble material was filtered off). The extract was washed with saturated brine, dried over anhydrous magnesium sulfate, and concentrated under reduced pressure to about 50 ml. The insoluble material was filtered off again. The filtrate was concentrated under reduced pressure, and the crystals were collected by filtration to give the object compound (1.09 g).

¹H-NMR (CDCl₃) δ 2.01 (1H, t), 2.22 (1H, dt), 2.78 (1H, d), 2.88 (1H, d), 2.96 (1H, dt), 3.12 (1H, dt), 3.19-3.27 (1H, m), 3.44-3.57 (4H, m), 3.85-3.96 (4H, m), 6.94 (1H, d), 7.05 (1H, dt), 7.22-7.32 (5H, m), 7.43 (1H, ddd), 8.13 (1H, dd), 8.18 (1H, t)

MS (ESI+, m/e) 340 (M+1)

In the same manner as in Reference Example 192, the following compounds (Reference Examples 193-194) were obtained.

### Reference Example 193

### N-{[(2R)-4-Benzylpiperazin-2-yl]methyl}benzamide

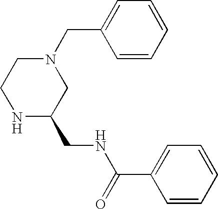

¹H-NMR (CDCl₃) δ 2.18 (1H, t), 2.30 (1H, t), 2.74 (1H, d), 2.88 (1H, d), 2.95 (1H, t), 3.14 (1H, d), 3.32-3.34 (1H, m), 3.47 (1H, d), 3.54 (1H, d), 3.60 (1H, d), 3.61 (1H, d), 5.47 (1H, br s), 7.26-7.49 (8H, m), 7.58 (1H, t), 7.80-7.82 (2H, m)

MS (ESI+, m/e) 310 (M+1)

### Reference Example 194

### N-{[(2R)-4-Benzylpiperazin-2-yl]methyl}-3,5-difluorobenzamide

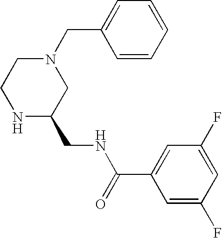

¹H-NMR (CDCl₃) δ 2.17 (1H, dd), 2.30 (1H, dt), 2.76 (1H, d), 2.86 (1H, d), 2.98 (1H, dt), 3.16 (1H, dt), 3.27-3.31 (1H, m), 3.47-3.59 (4H, m), 4.96 (1H, br s), 6.88-6.95 (1H, m), 7.24-7.34 (7H, m), 7.45 (1H, br t)

MS (ESI+, m/e) 346 (M+1)

### Reference Example 195

### N-{[(2R)-4-Benzylpiperazin-2-yl]methyl}cyclohexanecarboxamide

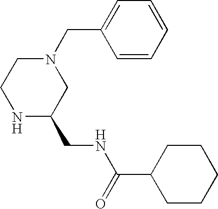

tert-Butyl (2S)-4-benzyl-2-{[(cyclohexylcarbonyl)(2,4-dimethoxybenzyl)amino]methyl}piperazine-1-carboxylate (2.26 g) was dissolved in dichloromethane (3.5 ml), TFA (15 ml) was added thereto, and the mixture was stirred at room temperature for 1.5 hr, and then at 70° C. for 10 min. The reaction mixture was poured into saturated aqueous sodium hydrogen carbonate by small portions. To the mixture was added potassium carbonate by small portions to basify the mixture, and the mixture was extracted with ethyl acetate (along with which the insoluble material was filtered off). The extract was washed with saturated brine, dried over anhydrous magnesium sulfate, and concentrated under reduced pressure to about 50 ml. The insoluble material was filtered off again. The filtrate was concentrated under reduced pressure, and the crystals were collected by filtration to give the object compound (473 mg).

¹H-NMR (CDCl₃) δ 1.17-1.85 (12H, m), 2.01-2.09 (2H, m), 2.68-2.74 (2H, m), 2.82-3.01 (3H, m), 3.16 (1H, ddd), 3.28 (1H, dt), 3.48 (2H, s), 5.88 (1H, br s), 7.23-7.34 (5H, m)

MS (ESI+, m/e) 316 (M+1)

### Reference Example 196

### tert-Butyl (2R)-4-benzyl-2-[(2E)-3-phenyl-2-propen-1-yl]piperazine-1-carboxylate

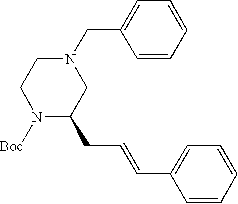

Diethyl benzylphosphonate (473 mg) was dissolved in THF (9 ml), the solution was ice-cooled, and sodium hydride (60% in oil) (113 mg) was added. The mixture was stirred at room temperature for 30 min, and ice-cooled again, and a solution of tert-butyl (2R)-4-benzyl-2-(2-oxoethyl)piperazine-1-carboxylate (600 mg) in THF (3 ml) was added. The mixture was further stirred at room temperature for 15 hr, and poured into saturated aqueous sodium hydrogen carbonate, and the mixture was extracted with ethyl acetate. The extract was washed successively with water and saturated brine, and dried over anhydrous magnesium sulfate, and the solvent was evaporated under reduced pressure. The residue was subjected to silica gel column chromatography, and the fraction eluted with ethyl acetate-hexane (1:9) was concentrated under reduced pressure to give the object compound (428 mg) as an oil.

¹H-NMR (CDCl₃) δ 1.40 (9H, s), 2.02-2.11 (2H, m), 2.61 (2H, t), 2.74 (1H, d), 2.80 (1H, d), 3.12 (1H, dt), 3.36 (1H, d), 3.57 (1H, d), 3.83-3.87 (1H, m), 4.09-4.13 (1H, m), 6.00-6.11 (1H, m), 6.32 (1H, d), 7.13-7.36 (10H, m)

MS (ESI+, m/e) 393 (M+1)

### Reference Example 197

### tert-Butyl (2R)-4-benzyl-2-[(E)-2-cyclopropylvinyl]piperazine-1-carboxylate

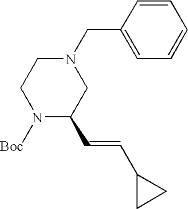

(Cyclopropylmethyl)(triphenyl)phosphonium bromide (385 mg) was dissolved in THF (10 ml), and the mixture was cooled to −78° C. N-Butyllithium (1.6M hexane solution, 1.25 ml) was added thereto, and the mixture was stirred at −20° C. for 20 min. A solution of tert-butyl (2S)-4-benzyl-2-formylpiperazine-1-carboxylate (608 mg) in THF (5 ml) was added thereto, and the mixture was further stirred at −20° C. for 2 hr. To the reaction mixture was added saturated aqueous ammonium chloride solution, and the mixture was extracted with ethyl acetate. The extract was dried over anhydrous sodium sulfate, and the solvent was evaporated under reduced pressure. The residue was subjected to silica gel column chromatography, and the fraction eluted with ethyl acetate-hexane (1:4) was concentrated under reduced pressure to give the object compound (700 mg) as an oil.

MS (ESI+, m/e) 343 (M+1)

### Reference Example 198

### Diethyl [2-(trifluoromethoxy)benzyl]phosphonate

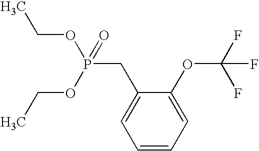

1-(Bromomethyl)-2-(trifluoromethoxy)benzene (1.37 g) and triethyl phosphite (1.2 ml) were dissolved in toluene (2.4 ml), and the mixture was heated under reflux for 15 hr. The reaction mixture was subjected to silica gel column chromatography, and the fraction eluted with ethyl acetate was concentrated under reduced pressure to give the object compound (1.77 g) as an oil.

¹H-NMR (CDCl₃) δ 1.25 (6H, t), 3.21 (1H, s), 3.28 (1H, s), 3.97-4.22 (4H, m), 7.19-7.34 (3H, m), 7.46-7.55 (1H, m)

In the same manner as in Reference Example 198, the following compounds (Reference Examples 199-200) were obtained.

### Reference Example 199

### Diethyl [3-(trifluoromethoxy)benzyl]phosphonate

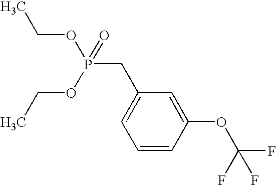

¹H-NMR (CDCl₃) δ 1.25 (6H, t), 3.12 (1H, s), 3.19 (1H, s), 3.97-4.18 (4H, m), 7.04-7.40 (4H, m)

### Reference Example 200

### Diethyl [4-(trifluoromethoxy)benzyl]phosphonate

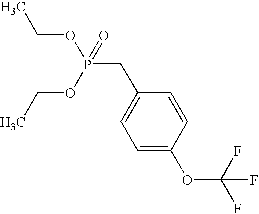

¹H-NMR (CDCl₃) δ 1.25 (6H, t), 3.11 (1H, s), 3.18 (1H, s), 3.95-4.19 (4H, m), 7.12-7.21 (2H, m), 7.29-7.37 (2H, m)

### Reference Example 201

### tert-Butyl (2R)-4-benzyl-2-[(E)-2-(2-fluorophenyl)vinyl]piperazine-1-carboxylate

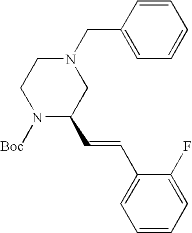

Diethyl (2-fluorobenzyl)phosphonate (500 mg) was dissolved in THF (10 ml), and the solution was ice-cooled. Sodium hydride (60% in oil) (112 mg) was added, and the mixture was stirred at room temperature for 30 min. The reaction mixture was ice-cooled again, a solution of tert-butyl (2S)-4-benzyl-2-formylpiperazine-1-carboxylate (562 mg) in THF (5 ml) was added, and the mixture was stirred at room temperature for 15 hr. The reaction mixture was poured into saturated aqueous sodium hydrogen carbonate, and the mixture was extracted with ethyl acetate. The extract was washed with brine, and dried over anhydrous sodium sulfate, and the solvent was evaporated under reduced pressure. The residue was subjected to silica gel column chromatography, and the fraction eluted with ethyl acetate-hexane (1:1) was concentrated under reduced pressure to give the object compound (943 mg) as an oil.

MS (ESI+, m/e) 397 (M+1)

In the same manner as in Reference Example 201, the following compounds (Reference Examples 202-209) shown in Table 1 were obtained.

TABLE 1

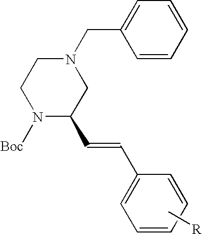

| Ref. Ex. | | | |
|---|---|---|---|
| No. | R | Compound | MS (ESI+) |
| 202 | 3-F | tert-Butyl (2R)-4-benzyl-2-[(E)-2- | 397 |
| | | (3-fluorophenyl)vinyl]piperazine-1- | |
| | | carboxylate | |
| 203 | 4-F | tert-Butyl (2R)-4-benzyl-2-[(E)-2- | 397 |
| | | (4-fluorophenyl)vinyl]piperazine-1- | |
| | | carboxylate | |
| 204 | 2-OCF₃ | tert-Butyl (2R)-4-benzyl-2-{(E)-2- | 463 |
| | | [2-(trifluoromethoxy)phenyl]vinyl} | |
| | | piperazine-1-carboxylate | |
| 205 | 3-OCF₃ | tert-Butyl (2R)-4-benzyl-2-{(E)-2- | 463 |
| | | [3-(trifluoromethoxy)phenyl]vinyl) | |
| | | piperazine-1-carboxylate | |
| 206 | 4-OCF₃ | tert-Butyl (2R)-4-benzyl-2-{(E)-2- | 463 |
| | | [4-(trifluoromethoxy)phenyl)vinyl} | |
| | | piperazine-1-carboxylate | |
| 207 | 2-CF₃ | tert-Butyl (2R)-4-benzyl-2-{(E)-2- | 447 |
| | | [2-(trifluoromethyl)phenyl]vinyl} | |
| | | piperazine-1-carboxylate | |
| 208 | 3-CF₃ | tert-Butyl (2R)-4-benzyl-2-{(E)-2- | 447 |
| | | [3-(trifluoromethyl)phenyl]vinyl} | |
| | | piperazine-1-carboxylate | |
| 209 | 4-CF₃ | tert-Butyl (2R)-4-benzyl-2-{(E)-2- | 447 |
| | | [4-(trifluoromethyl)phenyl]vinyl} | |
| | | piperazine-1-carboxylate | |

### Reference Example 210

### tert-Butyl (2R)-4-benzyl-2-[(E)-2-(pyridin-2-yl)vinyl]piperazine-1-carboxylate

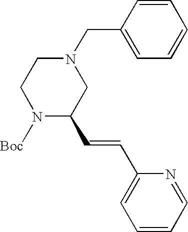

tert-Butyl (2S)-4-benzyl-2-formylpiperazine-1-carboxylate (500 mg) was dissolved in THF (5 ml), and the solution was cooled to 0° C. Triphenyl (pyridin-2-ylmethyl)phosphonium chloride-potassium hydride (1:1) (1059 mg) was added thereto, and the mixture was stirred at room temperature for 17 hr. To the reaction mixture was added saturated brine, and the mixture was extracted with ethyl acetate. The extract was dried over anhydrous magnesium sulfate, and concentrated under reduced pressure. The residue was subjected to basic silica gel column chromatography, and the fraction eluted with ethyl acetate-hexane (1:1) was concentrated under reduced pressure to give the object compound (590 mg) as an oil.

MS (ESI+, m/e) 380 (M+1)

### Reference Example 211

### (3R)-1-Benzyl-3-[(2E)-3-phenyl-2-propen-1-yl]piperazine

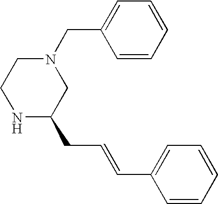

tert-Butyl (2R)-4-benzyl-2-[(2E)-3-phenyl-2-propen-1-yl]piperazine-1-carboxylate (424 mg) was dissolved in dichloromethane (1.5 ml), TFA (3 ml) was added thereto, and the mixture was stirred at room temperature for 40 min. The reaction mixture was poured into saturated aqueous sodium hydrogen carbonate by small portions. To the mixture was added potassium carbonate by small portions to basify the mixture, and the mixture was saturated with sodium chloride, and extracted with ethyl acetate. The extract was washed with saturated brine, and dried over anhydrous magnesium sulfate, and the solvent was evaporated under reduced pressure to give the object compound (315 mg) as an oil.

¹H-NMR (CDCl₃) δ 2.05 (1H, t), 2.21 (1H, dt), 2.40 (2H, t), 2.72 (1H, d), 2.85-3.09 (4H, m), 3.47 (1H, d), 3.56 (1H, d), 4.54 (1H, br s), 6.11 (1H, dt), 6.43 (1H, d), 7.16-7.33 (10H, m)

MS (ESI+, m/e) 293 (M+1)

In the same manner as in Reference Example 211, the following compound (Reference Example 212) was obtained.

### Reference Example 212

### (3R)-1-Benzyl-3-[(E)-2-cyclopropylvinyl]piperazine

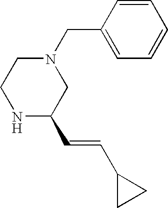

MS (ESI+, m/e) 243 (M+1)

In the same manner as in Reference Example 211, the following compounds (Reference Examples 213-221) shown in Table 2 were obtained.

TABLE 2

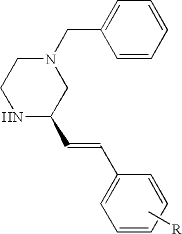

| Ref. Ex. | | | |
|---|---|---|---|
| No. | R | Compound | MS (ESI+) |
| 213 | 2-F | (3R)-1-Benzyl-3-[(E)-2-(2- | 297 |
| | | fluorophenyl)vinyl]piperazine | |
| 214 | 3-F | (3R)-1-Benzyl-3-[(E)-2-(3- | 297 |
| | | fluorophenyl)vinyl]piperazine | |
| 215 | 4-F | (3R)-1-Benzyl-3-[(E)-2-(4- | 297 |
| | | fluorophenyl)vinyl]piperazine | |
| 216 | 2-OCF₃ | (3R)-1-Benzyl-3-{(E)-2-[2- | 363 |
| | | (trifluoromethoxy)phenyl]vinyl} | |
| | | piperazine | |
| 217 | 3-OCF₃ | (3R)-1-Benzyl-3-{(E)-2-[3- | 363 |
| | | (trifluoromethoxy)phenyl]vinyl} | |
| | | piperazine | |
| 218 | 4-OCF₃ | (3R)-1-Benzyl-3-{(E)-2-[4- | 363 |
| | | (trifluoromethoxy)phenyl]vinyl} | |
| | | piperazine | |
| 219 | 2-CF₃ | (3R)-1-Benzyl-3-{(E)-2-[2- | 347 |
| | | (trifluoromethyl)phenyl]vinyl} | |
| | | piperazine | |
| 220 | 3-CF₃ | (3R)-1-Benzyl-3-{(E)-2-[3- | 347 |
| | | (trifluoromethyl)phenyl]vinyl} | |
| | | piperazine | |
| 221 | 4-CF₃ | (3R)-1-Benzyl-3-{(E)-2-[4- | 347 |
| | | (trifluoromethyl)phenyl]vinyl} | |
| | | piperazine | |

### Reference Example 222

### (3R)-1-Benzyl-3-[(E)-2-(pyridin-2-yl)vinyl]piperazine dihydrochloride

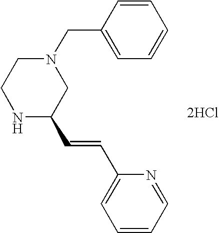

To tert-butyl (2R)-4-benzyl-2-[(E)-2-(pyridin-2-yl)vinyl]piperazine-1-carboxylate (280 mg) was added 4N hydrogen chloride-ethyl acetate solution (10 ml), and the mixture was stirred at room temperature for 3 hr, and concentrated under reduced pressure. The crystals were collected by filtration to give the object compound (260 mg).

MS (ESI+, m/e) 280 (M+1)

### Reference Example 223

### [(2R)-4-Benzyl-1-(tert-butoxycarbonyl)piperazin-2-yl]acetic acid

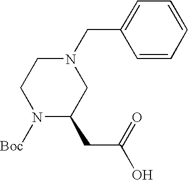

tert-Butyl (2R)-4-benzyl-2-(2-oxoethyl)piperazine-1-carboxylate (1.42 g) and 2-methyl-2-butene (4.6 ml) were dissolved in dioxane (17 ml), and a solution of sodium chlorite (2.22 g) and sodium dihydrogen phosphate (3.06 g) in water (11.5 ml) was added thereto. After stirring at room temperature for 1.5 hr, sodium chlorite (0.55 g) and sodium dihydrogen phosphate (0.55 g) were added thereto, and the mixture was further stirred at room temperature for 1 hr. The reaction mixture was poured into saturated brine, and the mixture was extracted with ethyl acetate. The extract was washed with saturated brine, and dried over anhydrous magnesium sulfate, and the solvent was evaporated under reduced pressure. The residue was subjected to silica gel column chromatography, and the fraction eluted with ethyl acetate-hexane (1:1-2:1) was concentrated under reduced pressure to give the object compound (882 mg) as an amorphous solid.

¹H-NMR (CDCl₃) δ 1.44 (9H, s), 2.16 (1H, dt), 2.38 (1H, dd), 2.65 (1H, dd), 2.86-2.99 (3H, m), 3.17-3.21 (2H, m), 3.57 (1H, d), 3.65 (1H, d), 3.88-3.92 (1H, m), 4.44 (1H, br s), 7.26-7.36 (5H, m)

MS (ESI+, m/e) 335 (M+1)

### Reference Example 224

### tert-Butyl (2R)-4-benzyl-2-[(5-phenyl-1,3,4-oxadiazol-2-yl)methyl]piperazine-1-carboxylate

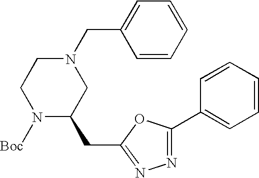

A mixture of [(2R)-4-benzyl-1-(tert-butoxycarbonyl)piperazin-2-yl]acetic acid (300 mg), 5-phenyl-1H-tetrazole (144 mg), DCC (204 mg) and toluene (6 ml) was stirred at 100° C. for 4 hr, and cooled to room temperature. The insoluble material was filtered, and washed with ethyl acetate. The filtrate was concentrated under reduced pressure. The residue was subjected to silica gel column chromatography, the fraction eluted with ethyl acetate-hexane (1:4) was concentrated under reduced pressure, and the crystals were collected by filtration to give the object compound (332 mg).

¹H-NMR (CDCl₃) δ 1.27 (9H, s), 2.09 (1H, t), 2.25 (1H, dd), 2.78-2.82 (2H, m), 3.22-3.26 (2H, m), 3.47 (1H, d), 3.56 (1H, d), 3.53-3.58 (1H, m), 4.04-4.10 (1H, m), 4.55-4.59 (1H, m), 7.22-7.34 (5H, m), 7.43-7.50 (3H, m), 7.96-7.99 (2H, m)

MS (ESI+, m/e) 435 (M+1)

### Reference Example 225

### (3R)-1-Benzyl-3-[(5-phenyl-1,3,4-oxadiazol-2-yl)methyl]piperazine

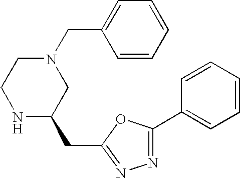

tert-Butyl (2R)-4-benzyl-2-[(5-phenyl-1,3,4-oxadiazol-2-yl)methyl]piperazine-1-carboxylate (332 mg) was dissolved in dichloromethane (1.5 ml), TFA (3 ml) was added thereto, and the mixture was stirred at room temperature for 50 min. The reaction mixture was poured into saturated aqueous sodium hydrogen carbonate by small portions. To the mixture was added potassium carbonate by small portions to basify the mixture, and the mixture was saturated with sodium chloride, and extracted with ethyl acetate. The extract was washed with saturated brine, and dried over anhydrous magnesium sulfate, and the solvent was evaporated under reduced pressure to give the object compound (254 mg) as an oil.

¹H-NMR (CDCl₃) δ 2.02 (1H, t), 2.13-2.21 (3H, m), 2.74 (1H, d), 2.86 (1H, d), 2.90-3.07 (3H, m), 3.32-3.41 (1H, m), 3.53 (2H, s), 7.22-7.32 (5H, m), 7.45-7.55 (3H, m), 7.98-8.01 (2H, m)

MS (ESI+, m/e) 335 (M+1)

### Reference Example 226

### 2-{[(2R)-4-Benzylpiperazin-2-yl]methyl}-1H-benzimidazole

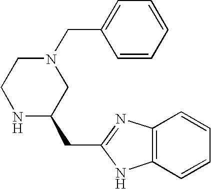

A solution of [(2R)-4-benzyl-1-(tert-butoxycarbonyl)piperazin-2-yl]acetic acid (576 mg), o-phenylenediamine (931 mg), WSC.HCl (660 mg) and HOBt (466 mg) in DMF (18 ml) was stirred at room temperature for 15 hr, and poured into saturated aqueous sodium hydrogen carbonate, and the mixture was extracted with ethyl acetate-THF (4:1). The extract was washed with saturated brine, and dried over anhydrous magnesium sulfate, and the solvent was evaporated under reduced pressure. The residue was dissolved in acetic acid (25 ml), and the solution was stirred at 65° C. for 3 hr, and concentrated under reduced pressure. TFA (5 ml) was added to the residue, and the mixture was stirred at room temperature for 1 hr. The reaction mixture was poured into saturated aqueous sodium hydrogen carbonate by small portions. To the mixture was added potassium carbonate by small portions to basify the mixture, and the mixture was saturated with sodium chloride, and extracted with ethyl acetate-THF (4:1). The extract was washed with saturated brine, and dried over anhydrous magnesium sulfate, and the solvent was evaporated under reduced pressure. The residue was subjected to basic silica gel column chromatography, and the fraction eluted with ethyl acetate-hexane-methanol (1:1:0-10:0:1) was concentrated under reduced pressure to give the object compound (290 mg) as an amorphous solid.

¹H-NMR (CDCl₃) δ 1.89 (1H, t), 2.10 (1H, dt), 2.73-2.83 (2H, m), 2.87-3.11 (4H, m), 3.24-3.32 (1H, m), 3.47 (2H, s), 7.17-7.33 (9H, m), 7.53 (2H, br s)

MS (ESI+, m/e) 307 (M+1)

### Reference Example 227

### Di-tert-butyl (2R)-2-[4-(ethoxycarbonyl)benzyl]piperazine-1,4-dicarboxylate

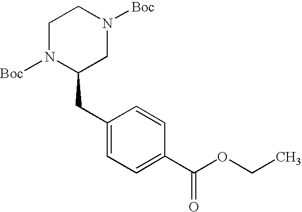

Di-tert-butyl (2R)-2-(4-{([(trifluoromethyl)sulfonyl]oxy}benzyl)piperazine-1,4-dicarboxylate (6.0 g), triethylamine (11 ml), palladium(II) acetate (510 mg) and dppf (1.26 g) were suspended in ethanol (65 ml), and the suspension was stirred at 80° C. for 12 hr under a carbon monoxide atmosphere. The reaction mixture was cooled to room temperature, diluted with ethyl acetate and water, and the insoluble material was filtered through celite. The organic layer was separated, washed with saturated brine, and dried over anhydrous magnesium sulfate. The solvent was evaporated under reduced pressure. The residue was subjected to silica gel column chromatography, the fraction eluted with ethyl acetate-hexane (1:4) was concentrated under reduced pressure, and the crystals were collected by filtration to give the object compound (4.1 g).

MS (ESI+, m/e) 449 (M+1)

### Reference Example 228

### tert-Butyl (3R)-3-[4-(2,2,2-trifluoro-1-hydroxyethyl)benzyl]piperazine-1-carboxylate

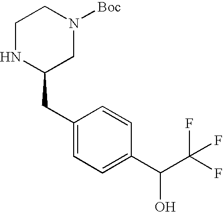

Di-tert-butyl (2R)-2-[4-(ethoxycarbonyl)benzyl]piperazine-1,4-dicarboxylate (1.79 g) was dissolved in ethanol (15 ml), pulverized potassium hydroxide (673 mg) was added, and the mixture was stirred at 80° C. for 30 min. The reaction mixture was concentrated under reduced pressure, and the residue was dissolved in water (5 ml). The mixture was weakly acidified (pH 3-4) with 10% aqueous citric acid solution, and extracted with ethyl acetate. The extract was washed with saturated brine, dried over anhydrous magnesium sulfate, and concentrated under reduced pressure to give 4-{[(2R)-1,4-bis(tert-butoxycarbonyl)piperazin-2-yl]methyl}benzoic acid (1.67 g) as crystals. 1.65 g therefrom was dissolved in THF (15 ml), the solution was ice-cooled, N-methylmorpholine (435 mg) and ethyl chloroformate (467 mg) were successively added. The mixture was stirred at 0-5° C. for 1 hr, and concentrated under reduced pressure, and the residue was dissolved in ethyl acetate (30 ml). The solution was washed successively with 6% aqueous sodium bicarbonate and water, dried over anhydrous magnesium sulfate, and concentrated under reduced pressure. The residue was subjected to silica gel column chromatography, and the fraction eluted with ethyl acetate-hexane (1:9-3:7) was concentrated under reduced pressure to give di-tert-butyl (2R)-2-(4-{([(ethoxycarbonyl)oxy]carbonyl}benzyl)piperazine-1,4-dicarboxylate (1.48 g) as an oil.

The total amount thereof was dissolved in THF (15 ml), and the solution was ice-cooled. Sodium borohydride (379 mg) was added, and then methanol (3 ml) was added dropwise over 5 min. The mixture was stirred at the same temperature for 30 min, and saturated aqueous ammonium chloride solution (5 ml) was added. The mixture was extracted with ethyl acetate, and the extract was dried over anhydrous magnesium sulfate, and concentrated under reduced pressure to give di-tert-butyl (2R)-2-[4-(hydroxymethyl)benzyl]piperazine-1,4-dicarboxylate (1.11 g) as an amorphous solid. 1.10 g therefrom was dissolved in dichloromethane (20 ml), manganese dioxide (2.35 g) was added thereto, and the mixture was stirred at room temperature for 15 hr. The insoluble material was filtered off, and the filtrate was concentrated under reduced pressure to give di-tert-butyl (2R)-2-(4-formylbenzyl)piperazine-1,4-dicarboxylate (1.01 g) as an oil. 1.00 g therefrom and trimethyl(trifluoromethyl)silane (702 mg) were dissolved in THF (10 ml), and TBAF (several mg) was added thereto. The mixture was stirred at room temperature for 2 hr, and concentrated under reduced pressure to give di-tert-butyl (2R)-2-[4-(2,2,2-trifluoro-1-hydroxyethyl)benzyl]piperazine-1,4-dicarboxylate (1.35 g) as an oil.

To the total amount thereof was added TFA (3 ml), and the mixture was stirred at room temperature for 30 min, and concentrated under reduced pressure. The residue was dissolved in THF (15 ml), and the solution was ice-cooled. N,N-Diisopropylethylamine (1.28 g) and di-tert-butyl bicarbonate (539 mg) were successively added, and the mixture was stirred at room temperature for 15 hr, and concentrated under reduced pressure. The residue was subjected to silica gel column chromatography, and the fraction eluted with ethyl acetate-methanol (9:1-7:3) was concentrated under reduced pressure to give the object compound (0.9 g) as an amorphous solid.

MS (ESI+, m/e) 375 (M+1)

### Reference Example 229

### tert-Butyl (3S)-4-benzyl-3-({[5-(methoxycarbonyl)pyridin-2-yl]oxy}methyl)piperazine-1-carboxylate

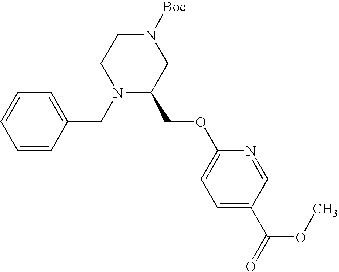

A mixture of tert-butyl (3S)-4-benzyl-3-(hydroxymethyl)piperazine-1-carboxylate (3.00 g), sodium hydride (60% in oil) (500 mg) and THF (50 ml) was stirred at room temperature for 1 hr, and ice-cooled, and methyl 6-chloronicotinate (1.68 g) was added. The reaction mixture was further stirred at room temperature for 2 hr, and poured into ice water, and the mixture was extracted with ethyl acetate. The extract was washed with saturated brine, and dried over anhydrous magnesium sulfate, and the solvent was evaporated under reduced pressure. The residue was subjected to silica gel column chromatography, and the fraction eluted with ethyl acetate-hexane (1:19-3:2) was concentrated under reduced pressure to give the object compound (2.83 g).

¹H-NMR (CDCl₃) δ 1.43 (9H, s), 2.31 (1H, br s), 2.75 (1H, dd), 2.91 (1H, br s), 3.45 (3H, br s), 3.58 (2H, br s), 3.91 (3H, s), 3.97-4.09 (1H, m), 4.50 (1H, d), 4.63 (1H, br s), 6.78 (1H, d), 7.21-7.36 (5H, m), 8.15 (1H, dd), 8.80 (1H, d)

MS (ESI+, m/e) 442 (M+1)

### Reference Example 230

### tert-Butyl (3S)-3-({[5-(methoxycarbonyl)pyridin-2-yl]oxy}methyl)piperazine-1-carboxylate

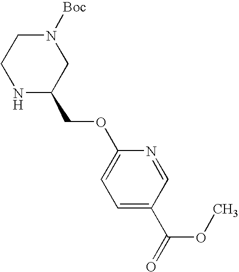

tert-Butyl (3S)-4-benzyl-3-({[5-(methoxycarbonyl)pyridin-2-yl]oxy}methyl)piperazine-1-carboxylate (1.00 g) was dissolved in methanol (30 ml), 20% palladium hydroxide-carbon (50% containing water, 150 mg) was added thereto, and the mixture was subjected to catalytic reduction at ambient temperature and normal pressure for 1 hr. The catalyst was filtered off, and the filtrate was concentrated under reduced pressure to give the object compound (747 mg).

¹H-NMR (CDCl₃) δ 1.47 (9H, s), 1.91 (1H, br s), 2.81 (1H, dd), 3.08 (2H, td), 2.96-3.12 (1H, m), 3.73 (2H, s), 3.91 (4H, s), 4.30 (1H, d), 4.36 (1H, d), 6.78 (1H, d), 8.16 (1H, dd), 8.80 (1H, d)

MS (ESI+, m/e) 352 (M+1)

### Reference Example 231

### tert-Butyl (3S)-3-[(2-cyanophenoxy)methyl]piperazine-1-carboxylate

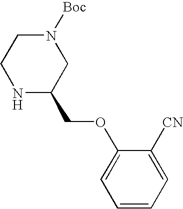

A mixture of tert-butyl (3S)-4-benzyl-3-(bromomethyl)piperazine-1-carboxylate (1.85 g), 2-cyanophenol (471 mg), potassium carbonate (1.04 mg) and DMF (5 ml) was stirred at 60° C. for 15 hr. Water was added to the reaction mixture, and the mixture was extracted with ethyl acetate. The extract was washed with saturated brine, dried over anhydrous magnesium sulfate, and concentrated under reduced pressure. The residue was subjected to silica gel column chromatography, and the fraction eluted with ethyl acetate-hexane (3:2) was concentrated under reduced pressure to give tert-butyl (3S)-4-benzyl-3-[(2-cyanophenoxy)methyl]piperazine-1-carboxylate (2.00 g) as an oil.

The total amount thereof was dissolved in 1,2-dichloromethane (50 ml), and the solution was ice-cooled. 1-Chloroethyl chloroformate (830 μl) was added thereto, and the mixture was stirred at 80° C. for 2 hr. After stirring, the solvent was evaporated under reduced pressure. Methanol (3 ml) was added to the residue, and the mixture was heated under reflux for 1 hr. The solvent was evaporated under reduced pressure. The residue was dissolved in ethyl acetate, and the solution was washed with saturated aqueous sodium hydrogen carbonate, and dried over anhydrous sodium sulfate. The solvent was evaporated under reduced pressure. The residue was suspended in THF (20 ml), N,N-diisopropylethylamine (3.4 ml) and di-tert-butyl bicarbonate (1.07 g) were added thereto, and the mixture was stirred at room temperature for 15 hr. The reaction mixture was partitioned between ethyl acetate and water. The organic layer was washed with saturated brine, and dried over anhydrous sodium sulfate, and the solvent was evaporated under reduced pressure. The residue was subjected to silica gel column chromatography, and the fraction eluted with ethyl acetate-methanol (19:1) was concentrated under reduced pressure to give the object compound (805 mg) as an amorphous solid.

MS (ESI+, m/e) 218 (M+1-“Boc”)

### Reference Example 232

### tert-Butyl (3S)-3-[(3,5-difluorophenoxy)methyl]piperazine-1-carboxylate

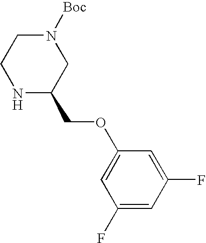

tert-Butyl (3S)-4-benzyl-3-(bromomethyl)piperazine-1-carboxylate (1.2 g) and 3,5-difluorophenol (509 mg) were dissolved in acetonitrile (30 ml), potassium carbonate (663 mg) was added thereto, and the mixture was stirred at room temperature for 8 hr. The reaction mixture was partitioned between ethyl acetate and water. The organic layer was washed with saturated brine, and dried over anhydrous sodium sulfate, and the solvent was evaporated under reduced pressure. The residue was subjected to silica gel column chromatography, and the fraction eluted with ethyl acetate-hexane (1:3) was concentrated under reduced pressure to give tert-butyl (3S)-4-benzyl-3-[(3,5-difluorophenoxy)methyl]piperazine-1-carboxylate (1.09 g) as an amorphous solid. The total amount thereof was dissolved in methanol (20 ml), 20% palladium hydroxide-carbon (50% containing water, 100 mg) was added thereto, and the mixture was subjected to catalytic reduction at ambient temperature and normal pressure for 12 hr. The catalyst was filtered off, and the filtrate was concentrated under reduced pressure to give the object compound (906 mg) as an amorphous solid.

MS (ESI+, m/e) 273 (M+1-“Boc”)

In the same manner as in Reference Example 232, the following compounds (Reference Examples 233-234) were obtained.

### Reference Example 233

### tert-Butyl (3S)-3-(phenoxymethyl)piperazine-1-carboxylate

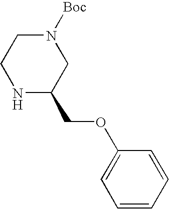

MS (ESI+, m/e) 293 (M+1)

### Reference Example 234

### tert-Butyl (3S)-3-[(2,6-difluorophenoxy)methyl]piperazine-1-carboxylate

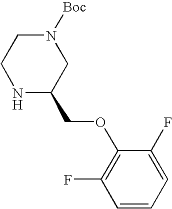

MS (ESI+, m/e) 329 (M+1)

### Reference Example 235

### tert-Butyl (3S)-3-[(4-methyl-1H-pyrazol-1-yl)methyl]piperazine-1-carboxylate

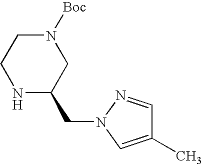

A solution of tert-butyl (3S)-4-benzyl-3-(bromomethyl)piperazine-1-carboxylate (370 mg) and 4-methylpyrazole (99 mg) in DMF (5 ml) was ice-cooled, and sodium hydride (60% in oil, 60 mg) was added thereto. The mixture was stirred at 0° C. for 15 min, and then at room temperature for 1 hr, and poured into ice-cooled saturated aqueous sodium hydrogen carbonate, and the mixture was extracted with ethyl acetate. The extract was dried over anhydrous sodium sulfate, and the solvent was evaporated under reduced pressure. The residue was subjected to silica gel column chromatography, and the fraction eluted with ethyl acetate-hexane (1:1) was concentrated under reduced pressure. The residue was dissolved in methanol (5 ml), 20% palladium hydroxide-carbon (50% containing water, 70 mg) was added thereto, and the mixture was subjected to catalytic reduction at ambient temperature and normal pressure for 2 hr. The catalyst was filtered off, and the filtrate was concentrated under reduced pressure to give the object compound (90 mg) as an oil.

MS (ESI+, m/e) 281 (M+1)

In the same manner as in Reference Example 235, the following compounds (Reference Examples 236-239) were obtained.

### Reference Example 236

### tert-Butyl (3S)-3-(1H-1,2,4-triazol-1-ylmethyl)piperazine-1-carboxylate

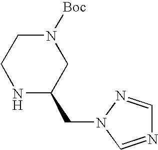

MS (ESI+, m/e) 268 (M+1)

### Reference Example 237

### tert-Butyl (3S)-3-(1H-pyrazol-1-ylmethyl)piperazine-1-carboxylate

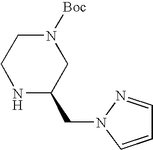

MS (ESI+, m/e) 267 (M+1)

### Reference Example 238

### tert-Butyl (3S)-3-(1H-indazol-1-ylmethyl)piperazine-1-carboxylate

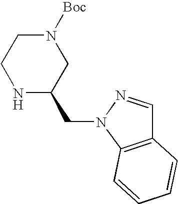

MS (ESI+, m/e) 317 (M+1)

### Reference Example 239

### tert-Butyl (3S)-3-(1H-1,2,3-benzotriazol-1-ylmethyl)piperazine-1-carboxylate

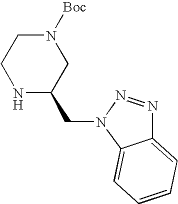

MS (ESI+, m/e) 318 (M+1)

### Reference Example 240

### tert-Butyl (3S)-3-(1H-imidazol-1-ylmethyl)piperazine-1-carboxylate

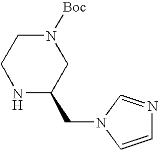

A solution of tert-butyl (3S)-4-benzyl-3-(bromomethyl)piperazine-1-carboxylate (600 mg) and imidazole (150 mg) in DMF (10 ml) was ice-cooled, and sodium hydride (60% in oil, 84 mg) was added thereto. The mixture was stirred at 0° C. for 15 min, and then at 60° C. for 1 hr, and poured into ice-cooled saturated aqueous sodium hydrogen carbonate, and the mixture was extracted with ethyl acetate. The extract was dried over anhydrous sodium sulfate, and the solvent was evaporated under reduced pressure. The residue was subjected to silica gel column chromatography, and the fraction eluted with ethyl acetate-hexane (1:1) was concentrated under reduced pressure. The residue was dissolved in methanol (5 ml), 20% palladium hydroxide-carbon (50% containing water, 100 mg) was added thereto, and the mixture was subjected to catalytic reduction at ambient temperature and normal pressure for 2 hr. The catalyst was filtered off, and the filtrate was concentrated under reduced pressure to give the object compound (210 mg) as an oil.

MS (ESI+, m/e) 267 (M+1)

In the same manner as in Reference Example 240, the following compound (Reference Example 241) was obtained.

### Reference Example 241

### tert-Butyl (3S)-3-[(3,5-dimethyl-1H-pyrazol-1-yl)methyl]piperazine-1-carboxylate

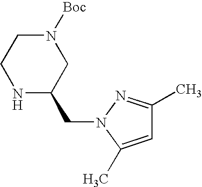

MS (ESI+, m/e) 295 (M+1)

### Reference Example 242

### tert-Butyl (3S)-3-{[3-(trifluoromethyl)-1H-pyrazol-1-yl]methyl}piperazine-1-carboxylate

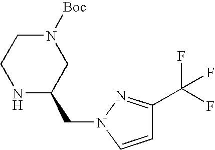

To a solution of tert-butyl (3S)-4-benzyl-3-(bromomethyl)piperazine-1-carboxylate (370 mg) and 3-trifluoromethylpyrazole (272 mg) in DMF (3 ml) was added BEMP (600 mg). The mixture was stirred at room temperature for 2 hr, and poured into ice-cooled saturated aqueous sodium hydrogen carbonate, and the mixture was extracted with ethyl acetate. The extract was dried over anhydrous sodium sulfate, and the solvent was evaporated under reduced pressure. The residue was subjected to silica gel column chromatography, and the fraction eluted with ethyl acetate-hexane (4:1) was concentrated under reduced pressure. The residue was dissolved in methanol (5 ml), 20% palladium hydroxide-carbon (50% containing water, 100 mg) was added thereto, and the mixture was subjected to catalytic reduction at ambient temperature and normal pressure for 2 hr. The catalyst was filtered off, and the filtrate was concentrated under reduced pressure to give the object compound (261 mg) as an oil.

MS (ESI+, m/e) 335 (M+1)

### Reference Example 243

### tert-Butyl (3S)-3-(1H-benzimidazol-1-ylmethyl)piperazine-1-carboxylate

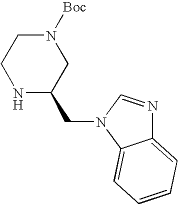

A mixture of tert-butyl (3S)-4-benzyl-3-(bromomethyl)piperazine-1-carboxylate (370 mg), 1H-benzimidazole (236 mg), potassium carbonate (690 mg) and DMF (5 ml) was stirred at 60° C. for 12 hr, and poured into water, and the mixture was extracted with ethyl acetate. The extract was washed with saturated brine, and dried over anhydrous sodium sulfate, and the solvent was evaporated under reduced pressure. The residue was subjected to silica gel column chromatography, and the fraction eluted with ethyl acetate-hexane (1:1) was concentrated under reduced pressure. The residue was dissolved in methanol (5 ml), 20% palladium hydroxide-carbon (50% containing water, 50 mg) was added thereto, and the mixture was subjected to catalytic reduction at ambient temperature and normal pressure for 12 hr. The catalyst was filtered off, and the filtrate was concentrated under reduced pressure to give the object compound (160 mg) as an oil.

MS (ESI+, m/e) 317 (M+1)

### Reference Example 244

### tert-Butyl (3S)-3-[(pyridin-2-yloxy)methyl]piperazine-1-carboxylate

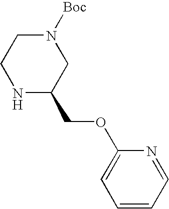

Potassium tert-butoxide (1.58 g) was dissolved in tert-butanol (60 ml), tert-butyl (3S)-4-benzyl-3-(hydroxymethyl)piperazine-1-carboxylate (3.06 g) and 2-bromopyridine (1.74 g) were added, and the mixture was stirred at 80° C. for 3 days. The reaction mixture was concentrated under reduced pressure, and the residue was partitioned between ethyl acetate and water. The organic layer was washed with saturated brine, and dried over anhydrous sodium sulfate, and the solvent was evaporated under reduced pressure. The residue was subjected to silica gel column chromatography, and the fraction eluted with ethyl acetate-hexane (3:7) was concentrated under reduced pressure to give tert-butyl (3S)-4-benzyl-3-[(pyridin-2-yloxy)methyl]piperazine-1-carboxylate (1.67 g) as an amorphous solid. The total amount thereof was dissolved in methanol (50 ml), 20% palladium hydroxide-carbon (50% containing water, 200 mg) was added thereto, and the mixture was subjected to catalytic reduction at ambient temperature and normal pressure for 12 hr. The catalyst was filtered off, and the filtrate was concentrated under reduced pressure to give the object compound (990 mg) as an amorphous solid.

MS (ESI+, m/e) 294 (M+1)

### Reference Example 245

### tert-Butyl (3R)-3-(3-methoxybenzyl)piperazine-1-carboxylate

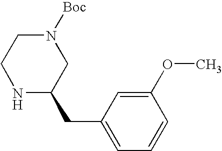

tert-Butyl (2S)-4-benzyl-2-formylpiperazine-1-carboxylate (1.00 g) was dissolved in THF (10 ml), and the solution was ice-cooled. 3-Methoxyphenylmagnesium bromide (1M THF solution, 4.0 ml) was added, and the mixture was stirred at room temperature for 15 hr. To the reaction mixture was added saturated aqueous ammonium chloride solution, and the mixture was extracted with ethyl acetate. The extract was washed with saturated brine, and dried over anhydrous sodium sulfate, and the solvent was evaporated under reduced pressure. The residue was subjected to silica gel column chromatography, and the fraction eluted with ethyl acetate-hexane (1:1) was concentrated under reduced pressure to give tert-butyl (2S)-4-benzyl-2-[(hydroxy) (3-methoxyphenyl)methyl]piperazine-1-carboxylate (1.26 g) as an amorphous solid.

The total amount thereof and lithium chloride (1.26 g) were suspended in 1,2-dichloroethane (15 ml), and the suspension was ice-cooled. Methanesulfonyl chloride (280 μl) and triethylamine (970 μl) were added thereto, and the mixture was stirred at room temperature for 15 hr. The reaction solution was partitioned between ethyl acetate and water. The organic layer was washed with saturated brine, and dried over anhydrous sodium sulfate, and the solvent was evaporated under reduced pressure. The residue was subjected to silica gel column chromatography, and the fraction eluted with ethyl acetate-hexane (3:2) was concentrated under reduced pressure to give (8aS)-7-benzyl-1-(3-methoxyphenyl)hexahydro[1,3]oxazolo[3,4-a]pyrazin-3-one (942 mg) as an amorphous solid. 900 mg therefrom was dissolved in ethanol-THF (1:1, 30 ml), 8N aqueous sodium hydroxide solution (5 ml) was added thereto, and the mixture was stirred at 50° C. for 24 hr. The reaction mixture was concentrated under reduced pressure, and the residue was dissolved in water (10 ml) and THF (10 ml). Benzyl chloroformate (420 μl) was added thereto, and the mixture was stirred at room temperature for 3 hr. The reaction mixture was extracted with ethyl acetate. The extract was washed with saturated brine, and dried over anhydrous sodium sulfate, and the solvent was evaporated under reduced pressure. The residue was subjected to silica gel column chromatography, and the fraction eluted with ethyl acetate-hexane (3:2) was concentrated under reduced pressure to give benzyl (2S)-4-benzyl-2-[(hydroxy)(3-methoxyphenyl)methyl]piperazine-1-carboxylate (469 mg) as an amorphous solid.

460 mg therefrom was dissolved in dichloromethane (10 ml), DAST (240 μl) was added thereto at −78° C., and the mixture was stirred at the same temperature for 3 hr. The reaction mixture was partitioned between ethyl acetate and water. The organic layer was washed with saturated brine, and dried over anhydrous sodium sulfate, and the solvent was evaporated under reduced pressure. The residue was subjected to silica gel column chromatography, and the fraction eluted with ethyl acetate-hexane (3:2) was concentrated under reduced pressure to give benzyl (2S)-4-benzyl-2-[(fluoro)(3-methoxyphenyl)methyl]piperazine-1-carboxylate (449 mg) as an amorphous solid.

300 mg therefrom was dissolved in ethanol (10 ml), 20% palladium hydroxide-carbon (50% containing water, 100 mg) was added thereto, and the mixture was subjected to catalytic reduction at ambient temperature and normal pressure for 12 hr. The catalyst was filtered off, and the filtrate was concentrated under reduced pressure to give (2R)-2-(3-methoxybenzyl)piperazine as an amorphous solid. The total amount thereof was dissolved in tert-butanol (5 ml) and water (4 ml), 8N aqueous sodium hydroxide solution (670 μl) and di-tert-butyl bicarbonate (146 mg) were added thereto, and the mixture was stirred at room temperature for 15 hr. The reaction mixture was partitioned between ethyl acetate and water. The organic layer was washed with saturated brine, and dried over anhydrous sodium sulfate, and the solvent was evaporated under reduced pressure. The residue was subjected to basic silica gel column chromatography, and the fraction eluted with ethyl acetate-methanol (17:3) was concentrated under reduced pressure to give the object compound (55 mg) as an oil.

MS (ESI+, m/e) 307 (M+1)

### Reference Example 246

### (3R)-1-Benzyl-3-[2-(cyclopropylmethoxy)ethyl]piperazine dihydrochloride

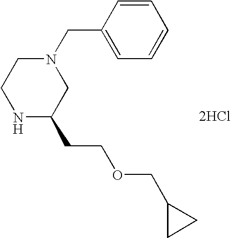

tert-Butyl (2R)-4-benzyl-2-(2-hydroxyethyl)piperazine-1-carboxylate (320 mg) was dissolved in DMF (5 ml), and the solution was ice-cooled. Sodium hydride (60% in oil, 48 mg) was added thereto, and the mixture was stirred at 0° C. for 10 min. After stirring, (bromomethyl)cyclopropane (120 μl) was added thereto, and the mixture was stirred at room temperature for 15 hr. The reaction mixture was partitioned between ethyl acetate and water. The organic layer was washed with saturated brine, and dried over anhydrous sodium sulfate, and the solvent was evaporated under reduced pressure. The residue was subjected to silica gel column chromatography, and the fraction eluted with ethyl acetate-hexane (3:7) was concentrated under reduced pressure to give tert-butyl (2R)-4-benzyl-2-[2-(cyclopropylmethoxy)ethyl]piperazine-1-carboxylate (150 mg) as an amorphous solid. To 140 mg therefrom was added 4N hydrogen chloride-ethyl acetate solution (5 ml), and the mixture was stirred at room temperature for 2 hr, and concentrated under reduced pressure to give the object compound (141 mg) as an amorphous solid.

MS (ESI+, m/e) 275 (M+1)

### Reference Example 247

### tert-Butyl (3S)-3-({[6-(trifluoromethyl)pyridin-2-yl]oxy}methyl)piperazine-1-carboxylate

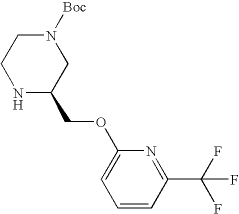

tert-Butyl (3S)-4-benzyl-3-(hydroxymethyl)piperazine-1-carboxylate (1.00 g) was dissolved in DMF (15 ml), and the solution was ice-cooled. Sodium hydride (60% in oil, 156 mg) was added thereto, and the mixture was stirred at 0° C. for 10 min. After stirring, 2-bromo-6-(trifluoromethyl)pyridine (884 mg) was added thereto, and the mixture was stirred at room temperature for 4 hr. The reaction solution was partitioned between ethyl acetate and water. The organic layer was washed with saturated brine, and dried over anhydrous sodium sulfate, and the solvent was evaporated under reduced pressure. The residue was subjected to silica gel column chromatography, and the fraction eluted with ethyl acetate-hexane (2:3) was concentrated under reduced pressure to give tert-butyl (3S)-4-benzyl-3-({[6-(trifluoromethyl)pyridin-2-yl]oxy}methyl)piperazine-1-carboxylate (1.44 g) as an amorphous solid. 1.41 g therefrom was dissolved in ethanol (50 ml), 20% palladium hydroxide-carbon (50% containing water, 300 mg) was added thereto, and the mixture was subjected to catalytic reduction at ambient temperature and normal pressure for 12 hr. The catalyst was filtered off, and the filtrate was concentrated under reduced pressure to give the object compound (937 mg) as an oil.

MS (ESI+, m/e) 362 (M+1)

In the same manner as in Reference Example 247, the following compound (Reference Example 248) was obtained.

### Reference Example 248

### tert-Butyl (3S)-3-({[4-(trifluoromethyl)pyridin-2-yl]oxy}methyl)piperazine-1-carboxylate

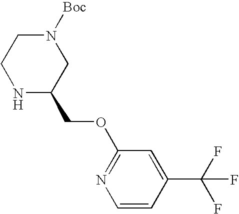

MS (ESI+, m/e) 362 (M+1)

### Reference Example 249

### tert-Butyl (3R)-3-{2-[4-(trifluoromethyl)phenyl]ethyl}piperazine-1-carboxylate

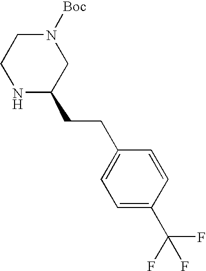

(2R)-1,4-Dibenzyl-2-vinylpiperazine (1.10 g) was dissolved in THF (10 ml), 9-BBN (0.5M THF solution, 30 ml) was added, and the mixture was stirred at room temperature for 12 hr. To the reaction mixture were added triphenylphosphine (168 mg), 1-iodo-4-(trifluoromethyl)benzene (1.53 g), tetrakis(triphenylphosphine)palladium(0) (92 mg) and 3N aqueous sodium hydroxide solution (3.1 ml), and the mixture was stirred at 70° C. for 24 hr. The solvent was evaporated under reduced pressure, 2N aqueous sodium hydroxide solution (80 ml) was added, and the mixture was stirred at room temperature for 1 hr. The reaction mixture was extracted with diethyl ether, and the organic layer was back-extracted with 1N hydrochloric acid. The acidic aqueous layer was separated, basified with 8N aqueous sodium hydroxide solution, and extracted with ethyl acetate. The extract was washed with saturated brine, and dried over anhydrous sodium sulfate, and the solvent was evaporated under reduced pressure. The residue was subjected to basic silica gel column chromatography, and the fraction eluted with ethyl acetate-hexane (3:7) was concentrated under reduced pressure to give (2R)-1,4-dibenzyl-2-{2-[4-(trifluoromethyl)phenyl]ethyl}piperazine (751 mg) as an amorphous solid.

The total amount thereof was dissolved in ethanol (20 ml), 20% palladium hydroxide-carbon (50% containing water, 100 mg) was added thereto, and the mixture was subjected to catalytic reduction at ambient temperature and normal pressure for 12 hr. The catalyst was filtered off, and the filtrate was concentrated under reduced pressure to give (2R)-2-{2-[4-(trifluoromethyl)phenyl]ethyl}piperazine as an amorphous solid. The total amount thereof was dissolved in tert-butanol (10 ml) and water (8 ml), 1N aqueous sodium hydroxide solution (1.71 ml) and di-tert-butyl bicarbonate (373 mg) were added thereto, and the mixture was stirred at room temperature for 15 hr. The reaction mixture was partitioned between ethyl acetate and water. The organic layer was washed with saturated brine, and dried over anhydrous sodium sulfate, and the solvent was evaporated under reduced pressure. The residue was subjected to basic silica gel column chromatography, and the fraction eluted with ethyl acetate-methanol (17:3) was concentrated under reduced pressure to give the object compound (455 mg) as an oil.

MS (ESI+, m/e) 359 (M+1)

### Reference Example 250

### tert-Butyl (2R)-2-(2-hydroxyethyl)piperazine-1-carboxylate

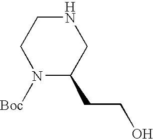

tert-Butyl (2R)-4-benzyl-2-(2-hydroxyethyl)piperazine-1-carboxylate (13.33 g) was dissolved in methanol (135 ml), 20% palladium hydroxide-carbon (50% containing water, 4.0 g) was added thereto, and the mixture was subjected to catalytic reduction at room temperature for 4 hr under moderate-pressure (5.0 kgf/cm²). The catalyst was filtered off, and the filtrate was concentrated under reduced pressure to give the object compound (9.44 g) as an oil.

¹H-NMR (CDCl₃) δ 1.47 (9H, s), 1.68 (1H, br s), 2.07-2.11 (1H, m), 2.36-2.40 (3H, m), 2.64-2.75 (1H, m), 2.85-2.96 (3H, m), 3.38-3.42 (1H, m), 3.66 (1H, dt), 3.82-3.86 (1H, m), 4.24 (1H, br s)

MS (ESI+, m/e) 231 (M+1)

### Reference Example 251

### 1-tert-Butyl 4-benzyl (2R)-2-(2-hydroxyethyl)piperazine-1,4-dicarboxylate

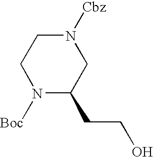

tert-Butyl (2R)-2-(2-hydroxyethyl)piperazine-1-carboxylate (9.44 g) was dissolved in dioxane (90 ml), and the solution was ice-cooled. A solution of sodium carbonate (4.78 g) in water (45 ml) and benzyl chloroformate (7.34 g) were added thereto, and the mixture was stirred at room temperature for 2 hr. The reaction mixture was poured into water, and the mixture was extracted with ethyl acetate. The extract was washed successively with water and saturated brine, and dried over anhydrous magnesium sulfate, and the solvent was evaporated under reduced pressure. The residue was subjected to silica gel column chromatography, and the fraction eluted with ethyl acetate-hexane (1:1-2:1) was concentrated under reduced pressure to give the object compound (14.17 g) as an oil.

MS (ESI+, m/e) 265 (M+1-“Boc”)

### Reference Example 252

### 1-tert-Butyl 4-benzyl (2R)-2-(2-bromoethyl)piperazine-1,4-dicarboxylate

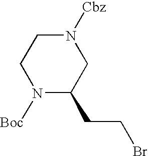

Triphenylphosphine (1.29 g) and carbon tetrabromide (1.63 g) were suspended in diethyl ether (20 ml), a solution of 1-tert-butyl 4-benzyl (2R)-2-(2-hydroxyethyl)piperazine-1,4-dicarboxylate (1.50 g) in diethyl ether (10 ml) was added, and the mixture was stirred at room temperature for 15 hr. To the reaction mixture was added THF (30 ml), triphenylphosphine (1.29 g) and carbon tetrabromide (1.63 g) were added thereto, and the mixture was further stirred at room temperature for 12 hr. The reaction mixture was concentrated under reduced pressure, and the residue was dissolved in THF (60 ml). Triphenylphosphine (1.29 g) and carbon tetrabromide (1.63 g) were further added thereto, and the mixture was stirred at room temperature for 3 days. The insoluble material was filtered off, and the filtrate was concentrated under reduced pressure. The residue was partitioned between ethyl acetate and water. The organic layer was washed with saturated brine, and dried over anhydrous sodium sulfate, and the solvent was evaporated under reduced pressure. The residue was subjected to silica gel column chromatography, and the fraction eluted with ethyl acetate-hexane (1:19-3:7) was concentrated under reduced pressure to give the object compound (697 mg) as an oil.

MS (ESI+, m/e) 427 (M+1)

### Reference Example 253

### 1-tert-Butyl 4-benzyl (2R)-2-[2-(4-methyl-1H-pyrazol-1-yl)ethyl]piperazine-1,4-dicarboxylate

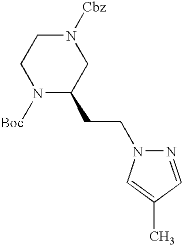

A mixture of 1-tert-butyl 4-benzyl (2R)-2-(2-bromoethyl)piperazine-1,4-dicarboxylate (320 mg), 4-methyl-1H-pyrazole (123 mg), potassium carbonate (415 mg) and DMF (5 ml) was stirred at 50° C. for 10 hr, and poured into water, and the mixture was extracted with ethyl acetate. The extract was washed with saturated brine, and dried over anhydrous sodium sulfate, and the solvent was evaporated under reduced pressure. The residue was subjected to silica gel column chromatography, and the fraction eluted with ethyl acetate-hexane (3:1) was concentrated under reduced pressure to give the object compound (330 mg) as an oil.

MS (ESI+, m/e) 429 (M+1)

### Reference Example 254

### 1-tert-Butyl 4-benzyl (2R)-2-{2-[(methylsulfonyl)oxy]ethyl}piperazine-1,4-dicarboxylate

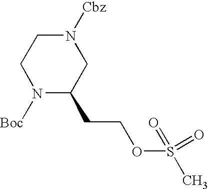

1-tert-Butyl 4-benzyl (2R)-2-(2-hydroxyethyl)piperazine-1,4-dicarboxylate (14.17 g) and triethylamine (5.90 g) were dissolved in THF (80 ml), and the solution was ice-cooled. Methanesulfonyl chloride (5.57 g) was added thereto, and the mixture was stirred at room temperature for 2 hr. The reaction mixture was poured into water, and the mixture was extracted with ethyl acetate. The extract was washed successively with water and saturated brine, and dried over anhydrous magnesium sulfate, and the solvent was evaporated under reduced pressure. The crystals were collected by filtration to give the object compound (15.54 g).

¹H-NMR (CDCl₃) δ 1.47 (9H, s), 1.88-2.04 (2H, m), 2.93-2.98 (5H, m), 3.95-4.33 (7H, m), 5.10 (1H, d), 5.17 (1H, d), 7.30-7.39 (5H, m)

MS (ESI+, m/e) 343 (M+1-“Boc”)

### Reference Example 255

### 1-tert-Butyl 4-benzyl (2R)-2-(2-phenoxyethyl)piperazine-1,4-dicarboxylate

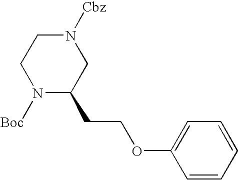

A mixture of 1-tert-butyl 4-benzyl (2R)-2-{2-[(methylsulfonyl)oxy]ethyl}piperazine-1,4-dicarboxylate (708 mg), phenol (188 mg), potassium carbonate (332 mg), potassium iodide (133 mg) and DMF (16 ml) was stirred at 65° C. for 15 hr, and poured into water, and the mixture was extracted with ethyl acetate. The extract was washed successively with water and saturated brine, and dried over anhydrous magnesium sulfate, and the solvent was evaporated under reduced pressure. The residue was subjected to basic silica gel column chromatography, and the fraction eluted with ethyl acetate-hexane (1:2) was concentrated under reduced pressure to give the object compound (591 mg) as an oil.

MS (ESI+, m/e) 441 (M+1)

In the same manner as in Reference Example 255, the following compounds (Reference Examples 256-320) shown in Table 3-1-Table 3-7 were obtained. In the column of “MS (ESI+)” in the Tables, “*” means that a mass value of “M+1-“Boc”” was obtained, and “**” means that a mass value of “M+1-“^{t}Bu”” was obtained (a mass value of M+1 was obtained for other compounds).

TABLE 3-1

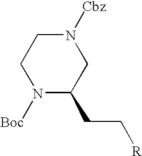

| Ref. Ex. No. | R | Compound | MS(ESI+) |
|---|---|---|---|
| 256 | 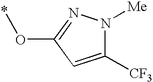 | 1-tert-Butyl 4-benzyl (2R)-2-(2-{[1- methyl-5-(trifluoromethyl)-1H-pyrazol- 3-yl]oxy}ethyl)piperazine-1,4- dicarboxylate | 513 |
| 257 | 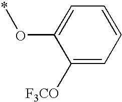 | 1-tert-Butyl 4-benzyl (2R)-2-{2-[2- (trifluoromethoxy)phenoxy]ethyl} piperazine-1,4-dicarboxylate | 525 |
| 258 | 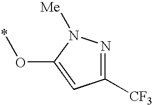 | tert-Butyl 4-benzyl (2R)-2-(2-{[1- methyl-3-(triofluoromethyl)-1H- pyrazol-5-yl]oxy}ethyl) piperzine-1,4-dicarboxylate | 513 |
| 259 | 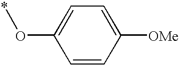 | 1-tert-Butyl 4-benzyl (2R)-2-[2-(4- methoxyphenoxy)ethyl]piperzine-1,4- dicarboxylate | 471 |
| 260 | 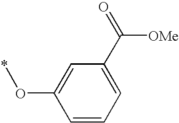 | 1-tert-Butyl 4-benzyl (2R)-2-{2-[3- (methoxycarbonyl)phenoxy]ethyl} piperazine-1,4-dicarboxylate | 499 |
| 261 | 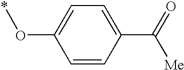 | 1-tert-Butyl 4-benzyl (2R)-2-[2-(4- acetyiphenoxy) ethyl]piperazine-1,4- dicarboxylate | 483 |
| 262 | 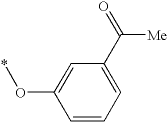 | 1-tert-Butyl 4-benzyl (2R)-2-[2-(3- acetylphenoxy)ethyl]piperzine-1,4- dicarboxylate | 483 |
| 363 | 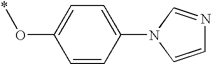 | 1-tert-Butyl 4-benzyl (2R)-2-{2- [4-(1H-imidazol-1- yl)phenoxy]ethyl}piperzine-1,4- dicarboxylate | 507 |
| 264 | 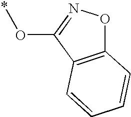 | 1-tert-Butyl 4-benzyl (2R)-2-[2-(1,2- benzisoxazol-3-yloxy)ethyl]piperzine- 1,4-dicarboxylate | 482 |

TABLE 3-2

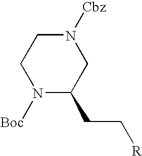

| Ref. Ex. No. | R | Compound | MS(ESI+) |
|---|---|---|---|
| 265 | 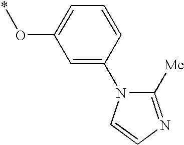 | 1-tert-Butyl 4-benzyl (2R)-2-{2-[3-(2- methyl-1H-midazol-1- yl)phenoxyl]ethyl}piperazine-1,4- dicarboxylate | 521 |
| 266 | 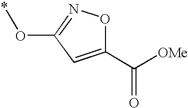 | 1-tert-Butyl 4-benzyl (2R)-2-(2-{[5- (methoxycarbonyl)-isoxazol-3- yl]oxy}ethyl)piperazine-1,4- dicarboxylate | 490 |
| 267 | 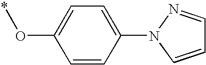 | 1-tert-Butyl 4-benzyl (2R)-2-{2-[4- (1H-pyrazol-1-yl)phenoxy]ethyl) piperazine-1,4-dicarboxylate | 407* |
| 268 | 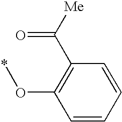 | 1-tert-Butyl 4-benzyl (2R)-2-[2-(2- acetylphenoxy)ethyl]piperazine-1,4- dicarboxylate | 483 |
| 269 | 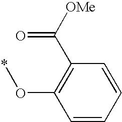 | 1-tert-Butyl 4-benzyl (2R)-2-{2-[2- (methoxycarbonyl)phenoxy]ethyl) piperazine-1,4-dicarboxylate | 499 |
| 270 | 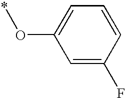 | 1-tert-Butyl 4-benzyl (2R)-2-[2-(3- fluorophenoxy)ethyl]piperazine-1,4- dicarboxylate | 459 |
| 271 | 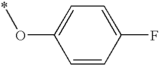 | 1-tert-Butyl 4-benzyl (2R)-2-[2-(4- fluorophenoxy)ethyl]piperazine-1,4- dicarboxylate | 459 |
| 272 | 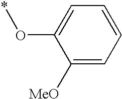 | 1-tert-Butyl 4-benzyl (2R)-2-[2-(2- methoxyphenoxy)ethyl]piperazine-1,4- dicarboxylate | 471 |
| 273 | 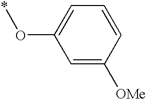 | 1-tert-Butyl 4-benzyl (2R)-2-[2-(3- methoxyphenoxy)ethyl]piperazine-1,4- dicarboxylate | 471 |
| 274 | 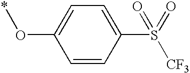 | 1-tert-Butyl 4-benzyl (2R)-2-(2-{4- [(trifluoromethyl)sulfonyl]phenoxy} ethyl) piperazine-1,4-dicarboxylate | 473* |

TABLE 3-3

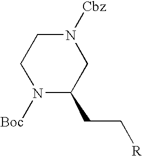

| Ref. Ex. No. | R | Compound | MS(ESI+) |
|---|---|---|---|
| 275 | 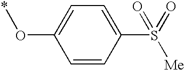 | 1-tert-Butyl 4-benzyl (2R)-2-{2-[4- (methylsulfonyl)phenoxy]ethyl} piperazine-1,4-dicarboxylate | 419* |
| 276 | 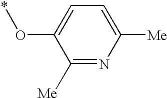 | 1-tert-Butyl 4-benzyl (2R)-2-{2- [(2,6-dimethylpyridin-3- yl)oxy]ethyl}piperazine-1,4- dicarboxylate | 470 |
| 277 | 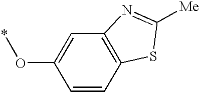 | 1-tert-Butyl 4-benzyl (2R)-2-{2-[(2- methyl-1,3-benzothiazol-5- yl)oxy]ethyl}piperazine-1,4- dicarboxylate | 512 |
| 278 | 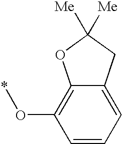 | 1-tert-Butyl 4-benzyl (2R)-2-{2- [(2,2-dimethyl-2,3-dihydro-1- benzofuran-7-yl)oxy]ethyl} piperazine-1,4-dicarboxylate | 511 |
| 279 | 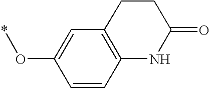 | 1-tert-Butyl 4-benzyl (2R)-2-{2-[(2- oxo-1,2,3,4-tetrahydroquinolin-6- yl)oxy]ethyl}piperazine-1,4- dicarboxylate | 510 |
| 280 | 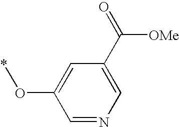 | 1-tert-Butyl 4-benzyl (2R)-2-(2-{[5- (methoxycarbonyl)pyridin-3- yl]oxy}ethyl)piperazine-1,4- dicarboxylate | 500 |
| 281 | 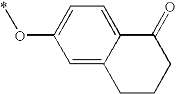 | 1-tert-Butyl 4-benzyl (2R)-2-{2-[(5- oxo-5,6,7,8-tetrahydronaphthalen-2- yl)oxy]ethyl}piperazine-1,4- dicarboxylate | 453** |
| 282 | 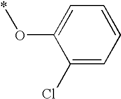 | 1-tert-Butyl 4-benzyl (2R)-2-[2-(2- chlorophenoxy)ethyl]piperazine-1,4- dicarboxylate | 475 |
| 283 | 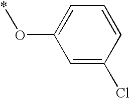 | 1-tert-Butyl 4-benzyl (2R)-2-[2-(3- chlorophenoxy)ethyl]piperazine-1,4- dicarboxylate | 475 |
| 284 | 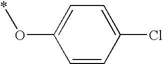 | 1-tert-Butyl 4-benzyl (2R)-2-[2-(4- chlorophenoxy)ethyl]piperazine-1,4- dicarboxylate | 475 |

TABLE 3-4

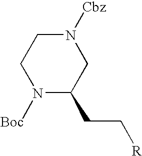

| Ref. Ex. No. | R | Compound | MS(ESI+) |
|---|---|---|---|
| 285 | 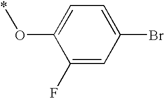 | 1-tert-Butyl 4-benzyl (2R)-2-[2-(4- bromo-2-fluorophenoxy)ethyl] piperazine-1,4-dicarboxylate | 538 |
| 286 | 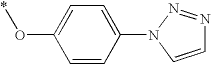 | 1-tert-Butyl 4-benzyl (2R)-2-{2-[4- (1H-1,2,3-triazol-1-yl) phenoxy]ethyl}piperzine- 1,4-dicarboxylate | 508 |
| 287 | 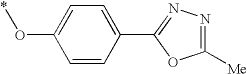 | 1-tert-Butyl 4-benzyl (2R)-2-{2-[4- (5-methyl-1,3,4-oxadiazol-2-yl) phenoxy]ethyl}piperzine- 1,4-dicarboxylate | 523 |
| 288 | 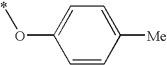 | 1-tert-Butyl 4-benzyl (2R)-2-[2-(4- methylphenoxy)-ethyl]piperazine- 1,4-dicarboxylate | 455 |
| 289 | 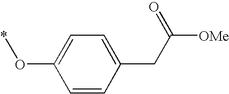 | 1-tert-Butyl 4-benzyl (2R)-2-{2-[4- (2-methoxy-2-oxoethyl)phenoxy] ethyl}piperazine-1,4-dicarboxylate | 513 |
| 290 | 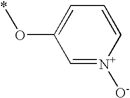 | 1-tert-Butyl 4-benzyl (2R)-2-{-2-[(1- oxidopyridin-3-yl)oxy]ethyl} piperazine-1,4-dicarboxylate | 458 |
| 291 | 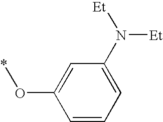 | 1-tert-Butyl 4-benzyl (2R)-2-{2-[3- (diethylamino)phenoxy]ethyl} piperazine-1,4-dicarboxylate | 512 |
| 292 | 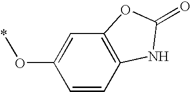 | 1-tert-Butyl 4-benzyl (2R)-2-{2-[(2- oxo-2,3-dihydro-1,3-benzoxazol-6- yl)oxy]ethyl}piperazine-1,4- dicarboxylate | 498 |
| 293 | 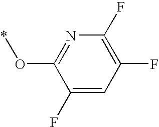 | 1-tert-Butyl 4-benzyl (2R)-2-{2- [(3,5,6-trifluoropyridin-2- yl)oxy]ethyl}piperzine-1,4- dicarboxylate | 396* |
| 294 | 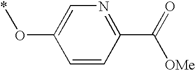 | 1-tert-Butyl 4-benzyl (2R)-2-(2-{[6- (methoxycarbonyl)pyridin-3- yl]oxy}ethyl)piperzine-1,4- dicarboxylate | 500 |

TABLE 3-5

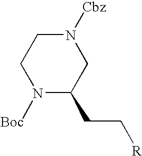

| Ref. Ex. No. | R | Compound | MS(ESI+) |
|---|---|---|---|
| 295 | 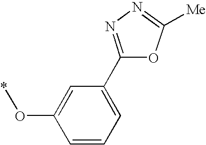 | 1-tert-Butyl 4-benzyl (2R)-2-{2-[3- (5-methyl-1,3,4-oxadiazol-2- yl)phenoxy]ethyl}piperazine-1,4- dicarboxylate | 523 |
| 296 | 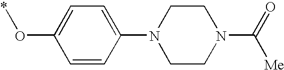 | 1-tert-Butyl 4-benzyl (2R)-2-(2-[4- (4-acetylpiperazin-1- yl)phenoxy]ethyl}piperazine-1,4- dicarboxylate | 567 |
| 297 | 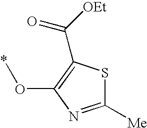 | 1-tert-Butyl 4-benzyl (2R)-2-(2- {[5-(ethoxycarbonyl)-2-methyl-1,3- thiazol-4-yl]oxy}ethyl) piperazine-1,4-dicarboxylate | 534 |
| 298 | 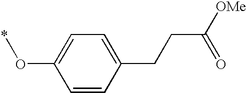 | 1-tert-Butyl 4-benzyl (2R)-2-{2-[4- (3-methoxy-3-oxopropyl)phenoxy] ethyl}piperazine-1,4-dicarboxylate | 527 |
| 299 | 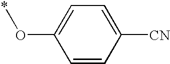 | 1-tert-Butyl 4-benzyl (2R)-2-[2-(4- cyanophenoxy)ethyl] piperazine-1,4-dicarboxylate | 466 |
| 300 | 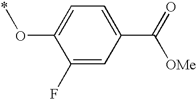 | 1-tert-Butyl 4-benzyl (2R)-2-{2-[2- fluoro-4-(methoxycarbonyl) phenoxy]ethyl}piperazine-1,4- dicarboxylate | 517 |
| 301 | 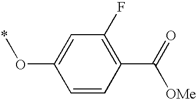 | 1-tert-Butyl 4-benzyl (2R)-2-{2-[3- fluoro-4-(methoxycarbonyl) phenoxy]ethyl}piperazine-1,4- dicarboxylate | 517 |
| 302 | 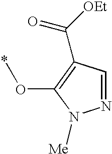 | 1-tert-Butyl 4-benzyl (2R)-2-(2- {[4-(ethoxycarbonyl)-1-methyl-1H- pyrazol-5-yl]oxy}ethyl) piperazine-1,4-dicarboxylate | 517 |
| 303 | 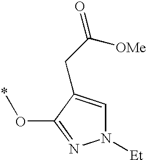 | 1-tert-Butyl 4-benzyl (2R)-2-(2- {[1-ethyl-4-(2-methoxy-2-oxo ethyl)-1H-pyrazol-3-yl]oxy}ethyl) piperazine-1,4-dicarboxylate | 531 |

TABLE 3-6

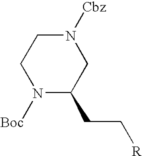

| Ref. Ex. No. | R | Compound | MS(ESI+) |
|---|---|---|---|
| 304 | 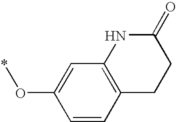 | 1-tert-Butyl 4-benzyl (2R)-2-{2-[(2- oxo-1,2,3,4-tetrahydroquinolin-7- yl)oxy]ethyl}piperazine-1,4- dicarboxylate | 410* |
| 305 | 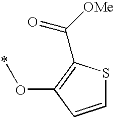 | 1-tert-Butyl 4-benzyl (2R)-2-(2-{[2- (methoxycarbonyl)-3- thienyl]oxy}ethyl)piperazine-1,4- dicarboxylate | 405* |
| 306 | 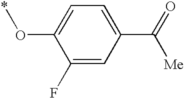 | 1-tert-Butyl 4-benzyl (2R)-2-[2-(4- acetyl-2-fluorophenoxy)ethyl] piperazine-1,4-dicarboxylate | 501 |
| 307 | 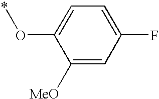 | 1-tert-Butyl 4-benzyl (2R)-2-[2-(4- fluoro-2-methoxyphenoxy)ethyl] piperazine-1,4-dicarboxylate | 489 |
| 308 | 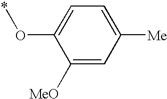 | 1-tert-Butyl 4-benzyl (2R)-2-[2-(2- methoxy-4-methylphenoxy)ethyl] piperazine-1,4-dicarboxylate | 485 |
| 309 | 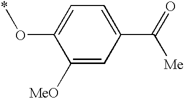 | 1-tert-Butyl 4-benzyl (2R)-2-[2-(4- acetyl-2-methoxyphenoxy)ethyl] piperazine-1,4-dicarboxylate | 513 |
| 310 | 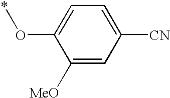 | 1-tert-Butyl 4-benzyl (2R)-2-[2-(4- cyano-2-methoxyphenoxy)ethyl] piperazine-1,4-dicarboxylate | 496 |
| 311 | 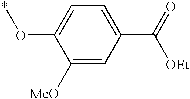 | 1-tert-Butyl 4-benzyl (2R)-2-{2-[4- (ethoxycarbonyl)-2-methoxyphenoxy] ethyl)piperazine-1,4-dicarboxylate | 543 |
| 312 | 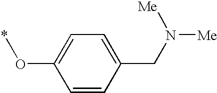 | 1-tert-Butyl 4-benzyl (2R)-2-(2-{4- [(dimethylamino)methyl]phenoxy} ethyl)piperazine-1,4-dicarboxylate | 498 |
| 313 | 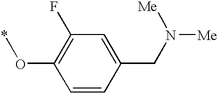 | 1-tert-Butyl 4-benzyl (2R)-2-(2-{4- [(dimethylamino)methyl]-2- fluorophenoxy}ethyl)piperazine-1,4- dicarboxylate | 516 |

TABLE 3-7

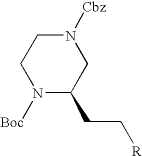

| Ref. Ex. No. | R | Compound | MS(ESI+) |
|---|---|---|---|
| 314 | 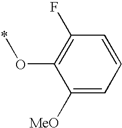 | 1-tert-Butyl 4-benzyl (2R)-2-[2-(2- fluoro-6-methoxyphenoxy)ethyl] piperazine-1,4-dicarboxylate | 489 |
| 315 | 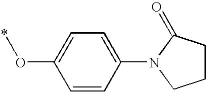 | 1-tert-Butyl 4-benzyl (2R)-2-{2-[4- (2-oxopyrrolidin-1-yl)phenoxy]ethyl} piperazine-1,4-dicarboxylate | 524 |
| 316 | 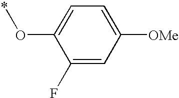 | 1-tert-Butyl 4-benzyl (2R)-2-[2-(2- fluoro-4-methoxyphenoxy)ethyl] piperazine-1,4-dicarboxylate | 489 |
| 317 | 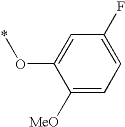 | 1-tert-Butyl 4-benzyl (2R)-2-[2-(5- fluoro-2-methoxyphenoxy)ethyl] piperazine-1,4-dicarboxylate | 489 |
| 318 | 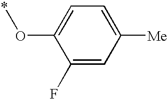 | 1-tert-Butyl 4-benzyl (2R)-2-[2-(2- fluoro-4-methylphenoxy)ethyl] piperazine-1,4-dicarboxylate | 473 |
| 319 | 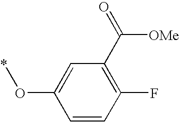 | 1-tert-Butyl 4-benzyl (2R)-2-{2- [4-fluoro-3-(methoxycarbonyl) phenoxy]ethyl} piperazine-1,4-dicarboxylate | 517 |
| 320 | 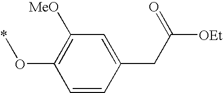 | 1-tert-Butyl 4-benzyl (2R)-2-{2- [4-(2-ethoxy-2-oxoethyl)-2-methoxy- phenoxy]ethyl}piperazine-1,4- dicarboxylate | 557 |

### Reference Example 321

### 1-tert-Butyl 4-benzyl (2R)-2-[2-(2-fluorophenoxy)ethyl]piperazine-1,4-dicarboxylate

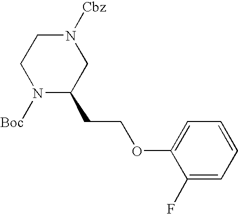

A mixture of 1-tert-butyl 4-benzyl (2R)-2-{2-[(methylsulfonyl)oxy]ethyl}piperazine-1,4-dicarboxylate (221 mg), 2-fluorophenol (84 mg), potassium carbonate (138 mg), potassium iodide (83 mg) and DMF (5 ml) was stirred at 65° C. for 15 hr. Saturated brine was added to the reaction mixture, and the liberated oil was extracted with ethyl acetate. The extract was washed successively with 6% aqueous sodium bicarbonate, 10% aqueous citric acid solution and saturated brine, dried over anhydrous magnesium sulfate, and concentrated under reduced pressure. The residue was subjected to basic silica gel column chromatography, and the fraction eluted with ethyl acetate-hexane (1:9-3:7) was concentrated under reduced pressure to give the object compound (210 mg) as an oil.

MS (ESI+, m/e) 459 (M+1)

### Reference Example 322

### 1-tert-Butyl 4-benzyl (2R)-2-{2-[4-(methoxycarbonyl)phenoxy]ethyl}piperazine-1,4-dicarboxylate

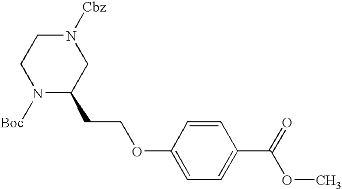

1-tert-Butyl 4-benzyl (2R)-2-{2-[(methylsulfonyl)oxy]ethyl}piperazine-1,4-dicarboxylate (1.11 g) was dissolved in DMF (10 ml), methyl 4-hydroxybenzoate (681 mg), potassium carbonate (1.38 g) and potassium iodide (415 mg) were added, and the mixture was stirred at 60° C. for 15 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate was concentrated under reduced pressure to give the object compound (452 mg) as an oil.

MS (ESI+, m/e) 499 (M+1)

### Reference Example 323

### 1-tert-Butyl 4-benzyl (2R)-2-(2-{[2-(methoxycarbonyl)pyridin-3-yl]oxy}ethyl)piperazine-1,4-dicarboxylate

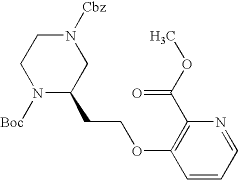

A mixture of 1-tert-butyl 4-benzyl (2R)-2-{2-[(methylsulfonyl)oxy]ethyl}piperazine-1,4-dicarboxylate (708 mg), methyl 3-hydroxypyridine-2-carboxylate (490 mg), potassium carbonate (332 mg), potassium iodide (266 mg) and DMF (16 ml) was stirred at 65° C. for 15 hr, and poured into water, and the mixture was extracted with ethyl acetate. The extract was ice-cooled, and washed successively with 0.5N aqueous sodium hydroxide solution, water and saturated brine, and dried over anhydrous magnesium sulfate. The solvent was evaporated under reduced pressure. The residue was subjected to basic silica gel column chromatography, and the fraction eluted with ethyl acetate-hexane (1:2) was concentrated under reduced pressure to give the object compound (658 mg) as an oil.

MS (ESI+, m/e) 500 (M+1)

In the same manner as in Reference Example 323, the following compounds (Reference Examples 324-335) shown in Table 4-1-Table 4-2 were obtained. In the column of “MS (ESI+)” in the Tables, “*” means that a mass value of “M+1-“Boc”” was obtained (a mass value of M+1 was obtained for other compounds).

TABLE 4-1

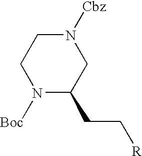

| Ref. Ex. No. | R | Compound | MS(ESI+) |
|---|---|---|---|
| 324 | 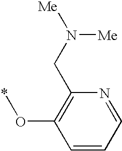 | 1-tert-Butyl 4-benzyl (2R)-2-[2-({2- [(dimethylamino)methyl]pyridin-3- yl}oxy)ethyl]piperazine-1,4- dicarboxylate | 499 |
| 325 | 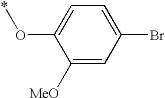 | 1-tert-Butyl 4-benzyl (2R)-2-[2-(4- bromo-2-methoxyphenoxy)ethyl] piperazine-1,4-dicarboxylate | 449* |
| 326 | 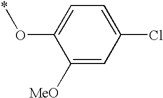 | 1-tert-Butyl 4-benzyl (2R)-2-[2-(4- chloro-2-methoxyphenoxy)ethyl] piperazine-1,4-dicarboxylate | 405* |
| 327 | 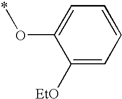 | 1-tert-Butyl 4-benzyl (2R)-2-[2-(2- ethoxyphenoxy)ethyl]piperazine-1,4- dicarboxylate | 385* |
| 328 | 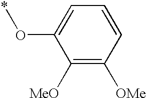 | 1-tert-Butyl 4-benzyl (2R)-2-[2-(2,3- dimethoxyphenoxy)ethyl] piperazine-1,4-dicarboxylate | 401* |
| 329 | 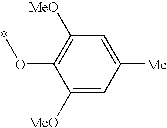 | 1-tert-Butyl 4-benzyl (2R)-2-[2-(2,6- dimethoxy-4-methylphenoxy) ethyl]piperazine-1,4-dicarboxylate | 415* |
| 330 | 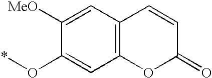 | 1-tert-Butyl 4-benzyl (2R)-2-{2-[(6- methoxy-2-oxo-2H-chromen-7- yl)oxy]ethyl}piperazine-1,4- dicarboxylate | 439* |
| 331 | 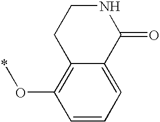 | 1-tert-Butyl 4-benzyl (2R)-2-{2-[(1- oxo-1,2,3,4-tetrahydro isoquinolin-5-yl)oxy]ethyl} piperazine-1,4-dicarboxylate | 510 |
| 332 | 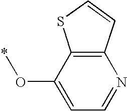 | 1-tert-Butyl 4-benzyl (2R)-2-[2- (thieno[3,2-b]pyridin-7- yloxy)ethyl]piperazine-1,4- dicarboxylate | 498 |

TABLE 4-2

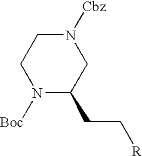

| Ref. Ex. No. | R | Compound | MS(ESI+) |
|---|---|---|---|
| 333 | 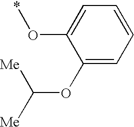 | 1-tert-Butyl 4-benzyl (2R)-2-[2-(2- isopropoxyphenoxy)ethyl]piperazine- 1,4-dicarboxylate | 399* |
| 334 | 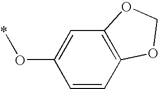 | 1-tert-Butyl 4-benzyl (2R)-2-[2- (1,3-benzodioxol-5-yloxy)ethyl] piperazine-1,4-dicarboxylate | 385* |
| 335 | 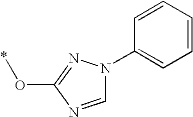 | 1-tert-Butyl 4-benzyl (2R)-2-{2-[(1- phenyl-1H-1,2,4-triazol-3- yl)oxy]ethyl}piperazine-1,4- dicarboxylate | 508 |

### Reference Example 336

### 2-Fluoro-4-(5-methyl-1,3,4-oxadiazol-2-yl)phenol

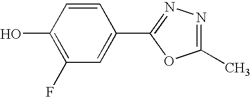

Methyl 3-fluoro-4-hydroxybenzoate (1.0 g) was dissolved in ethanol (10 ml), hydrazine monohydrate (2.9 g) was added thereto, and the mixture was heated under reflux for 12 hr. The solvent was evaporated under reduced pressure, triethyl orthoformate (10 ml) was added thereto, and the mixture was heated under reflux for 12 hr. The reaction mixture was partitioned between ethyl acetate and water. The organic layer was washed with saturated brine, and dried over anhydrous sodium sulfate, and the solvent was evaporated under reduced pressure. The residue was suspended in diisopropyl ether, and the precipitated crystals were collected by filtration to give the object compound (755 mg).

¹H-NMR (DMSO-d₆) δ 2.11 (3H, s), 6.61-6:75 (2H, m), 7.73 (1H, t), 10.54 (1H, br s)

MS (ESI+, m/e) 195 (M+1)

In the same manner as in Reference Example 336, the following compounds (Reference Examples 337-340) were obtained.

### Reference Example 337

### 3-Fluoro-4-(5-methyl-1,3,4-oxadiazol-2-yl)phenol

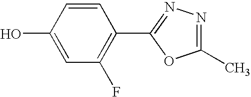

¹H-NMR (DMSO-d₆) δ 2.55 (3H, s), 7.13 (1H, t), 7.56-7.75 (2H, m), 10.79 (1H, br s)

MS (ESI+, m/e) 195 (M+1)

### Reference Example 338

### 4-Fluoro-3-(5-methyl-1,3,4-oxadiazol-2-yl)phenol

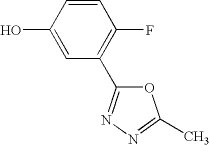

¹H-NMR (DMSO-d₆) δ 2.59 (3H, s), 6.95-7.07 (1H, m), 7.22-7.38 (2H, m), 9.92 (1H, br s)

MS (ESI+, m/e) 195 (M+1)

### Reference Example 339

### 3-Methoxy-4-(5-methyl-1,3,4-oxadiazol-2-yl)phenol

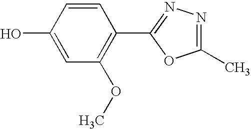

¹H-NMR (DMSO-d₆) δ 2.55 (3H, s), 3.86 (3H, s), 6.94 (1H, d), 7.37-7.44 (2H, m), 9.88 (1H, s)

MS (ESI+, m/e) 207 (M+1)

### Reference Example 340

### 2-Methoxy-5-(5-methyl-1,3,4-oxadiazol-2-yl)phenol

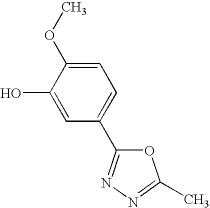

¹H-NMR (DMSO-d₆) δ 2.54 (3H, s), 3.84 (3H, s), 7.09 (1H, d), 7.35-7.51 (2H, m), 9.59 (1H, br s)

MS (ESI+, m/e) 207 (M+1)

### Reference Example 341

### 1-tert-Butyl 4-benzyl (2R)-2-{2-[2-methoxy-5-(methoxycarbonyl)phenoxy]ethyl}piperazine-1,4-dicarboxylate

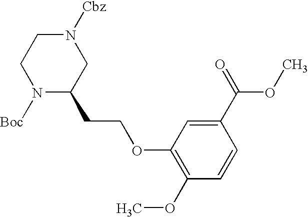

1-tert-Butyl 4-benzyl (2R)-2-{2-[(methylsulfonyl)oxy]ethyl}piperazine-1,4-dicarboxylate (442 mg) was dissolved in DMA (10 ml), and methyl 3-hydroxy-4-methoxybenzoate (273 mg) and cesium carbonate (652 mg) were added thereto. The mixture was stirred at 60° C. for 15 hr, and the reaction solution was partitioned between ethyl acetate and water. The organic layer was washed with saturated brine, and dried over anhydrous sodium sulfate, and the solvent was evaporated under reduced pressure. The residue was subjected to silica gel column chromatography, and the fraction eluted with ethyl acetate-hexane (7:3) was concentrated under reduced pressure to give the object compound (482 mg) as a colorless amorphous solid.

MS (ESI+, m/e) 429 (M+1-“Boc”)

In the same manner as in Reference Example 341, the following compounds (Reference Examples 342-346) were obtained.

### Reference Example 342

### 1-tert-Butyl 4-benzyl (2R)-2-{2-[2-fluoro-4-(5-methyl-1,3,4-oxadiazol-2-yl)phenoxy]ethyl}piperazine-1,4-dicarboxylate

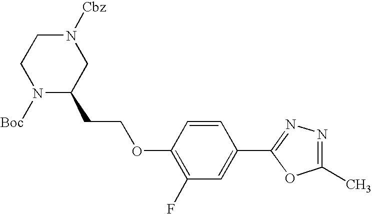

MS (ESI+, m/e) 541 (M+1)

### Reference Example 343

### 1-tert-Butyl 4-benzyl (2R)-2-{2-[3-fluoro-4-(5-methyl-1,3,4-oxadiazol-2-yl)phenoxy]ethyl}piperazine-1,4-dicarboxylate

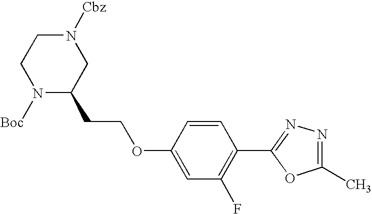

MS (ESI+, m/e) 541 (M+1)

### Reference Example 344

### 1-tert-Butyl 4-benzyl (2R)-2-{2-[4-fluoro-3-(5-methyl-1,3,4-oxadiazol-2-yl)phenoxy]ethyl}piperazine-1,4-dicarboxylate

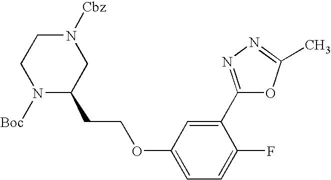

MS (ESI+, m/e) 541 (M+1)

### Reference Example 345

### 1-tert-Butyl 4-benzyl (2R)-2-{2-[2-methoxy-4-(5-methyl-1,3,4-oxadiazol-2-yl)phenoxy]ethyl}piperazine-1,4-dicarboxylate

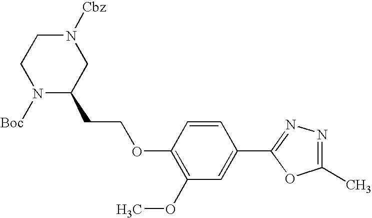

MS (ESI+, m/e) 553 (M+1)

### Reference Example 346

### 1-tert-Butyl 4-benzyl (2R)-2-{2-[2-methoxy-5-(5-methyl-1,3,4-oxadiazol-2-yl)phenoxy]ethyl}piperazine-1,4-dicarboxylate

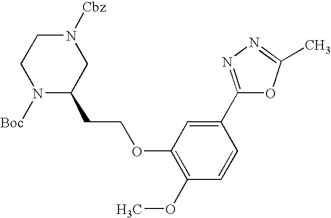

MS (ESI+, m/e) 553 (M+1)

### Reference Example 347

### 1-tert-Butyl 4-benzyl (2R)-2-[2-(1H-benzimidazol-1-yl)ethyl]piperazine-1,4-dicarboxylate

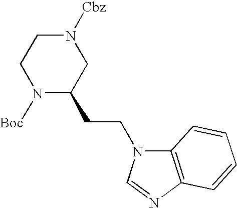

A mixture of 1-tert-butyl 4-benzyl (2R)-2-{2-[(methylsulfonyl)oxy]ethyl}piperazine-1,4-dicarboxylate (619 mg), 1H-benzimidazole (331 mg), potassium carbonate (1.20 g) and DMF (7 ml) was stirred at 50° C. for 10 hr, and poured into water, and the mixture was extracted with ethyl acetate. The extract was washed with saturated brine, and dried over anhydrous sodium sulfate, and the solvent was evaporated under reduced pressure. The residue was subjected to silica gel column chromatography, and the fraction eluted with ethyl acetate-hexane (1:4-1:1) was concentrated under reduced pressure to give the object compound (510 mg) as an oil.

MS (ESI+, m/e) 465 (M+1)

### Reference Example 348

### 1-tert-Butyl 4-benzyl (2R)-2-[2-(3,5-di-tert-butyl-1H-pyrazol-1-yl)ethyl]piperazine-1,4-dicarboxylate

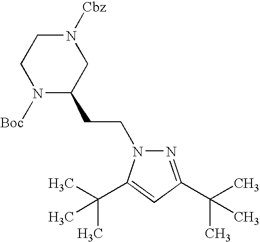

3,5-Di-tert-butyl-1H-pyrazole (204 mg) was dissolved in DMF (7 ml), and the solution was ice-cooled. Sodium hydride (60% in oil, 46 mg) was added thereto, and the mixture was stirred at 0° C. for 15 min. After stirring, 1-tert-butyl 4-benzyl (2R)-2-{2-[(methylsulfonyl)oxy]ethyl}piperazine-1,4-dicarboxylate (250 mg) was added thereto, and the mixture was stirred at room temperature for 1 hr. The reaction mixture was poured into ice-cooled saturated aqueous sodium hydrogen carbonate, and the mixture was extracted with ethyl acetate. The extract was dried over anhydrous sodium sulfate, and the solvent was evaporated under reduced pressure. The residue was subjected to silica gel column chromatography, and the fraction eluted with ethyl acetate-hexane (1:1) was concentrated under reduced pressure to give the object compound (220 mg) as an oil.

MS (ESI+, m/e) 527 (M+1)

In the same manner as in Reference Example 347 or Reference Example 348, the following compounds (Reference Examples 349-363) shown in Table 5-1-Table 5-2 were obtained. In the column of “Base” in the Tables, the compounds described as “K₂CO₃” were synthesized according to the method of Reference Example 347 and the compounds described as “NaH” were synthesized according to the method of Reference Example 348. In addition, in the column of “MS (ESI+)” in the Tables, “*” means that a mass value of “M+1-“Boc”” was obtained, and “**” means that a mass value of “M+1-“^{t}Bu”” was obtained (a mass value of M+1 was obtained for other compounds).

TABLE 5-1

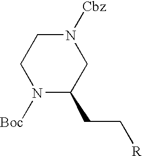

| Ref. Ex. No. | R | Compound | Base | MS(ESI+) |
|---|---|---|---|---|
| 349 | 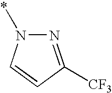 | 1-tert-Butyl 4-benzyl (2R)-2-{2-[3- (trifluoromethyl)-1H-pyrazol-1- yl]ethyl}piperazine-1,4- dicarboxylate | K₂CO₃ | 483 |
| 350 | 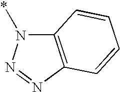 | 1-tert-Butyl 4-benzyl (2R)-2-[2- (1H-1,2,3-benzotriazol-1-yl)ethyl] piperazine-1,4-dicarboxylate | K₂CO₃ | 466 |
| 351 | 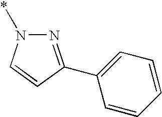 | 1-tert-Butyl 4-benzyl (2R)-2-[2-(3- phenyl-1H-pyrazol-1-yl)ethyl] piperazine-1,4-dicarboxylate | K₂CO₃ | 491 |
| 352 | 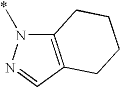 | 1-tert-Butyl 4-benzyl (2R)-2-[2- (4,5,6,7-tetrahydro-1H-indazol-1- yl)ethyl]piperazine-1,4- dicarboxylate | K₂CO₃ | 469 |
| 353 | 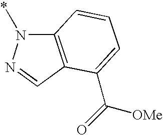 | 1-tert-Butyl 4-benzyl (2R)-2-{2-[4- (methoxycarbonyl)-1H-indazol-1-yl] ethyl}piperazine-1,4-dicarboxylate | K₂CO₃ | 523 |
| 354 | 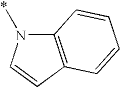 | 1-tert-Butyl 4-benzyl (2R)-2-[2- (1H-indol-1-yl)ethyl]piperazine- 1,4-dicarboxylate | NaH | 364* |
| 355 | 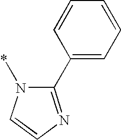 | 1-tert-Butyl 4-benzyl (2R)-2-[2-(2- phenyl-1H-imidazol-1-yl)ethyl] piperazine-1,4-dicarboxylate | NaH | 491 |
| 356 | 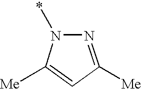 | 1-tert-Butyl 4-benzyl (2R)-2-[2- (3,5-dimethyl-1H-pyrazol-1-yl)ethyl] piperazine-1,4-dicarboxylate | K₂CO₃ | 443 |
| 357 | 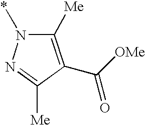 | 1-tert-Butyl 4-benzyl (2R)-2-{2-[4- (methoxycarbonyl)-3,5-dimethyl-1H- pyrazol-1-yl]ethyl}piperazine- 1,4-dicarboxylate | NaH | 501 |

TABLE 5-2

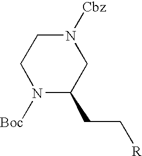

| Ref. Ex. No. | R | Compound | Base | MS(ESI+) |
|---|---|---|---|---|
| 358 | 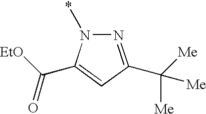 | 1-tert-Butyl 4-benzyl (2R)-2-{2- [3-tert-butyl-5-(ethoxycarbonyl)- 1H-pyrazol-1-yl]ethyl}piperazine- 1,4-dicarboxylate | NaH | 543 |
| 359 | 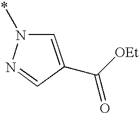 | 1-tert-Butyl 4-benzyl (2R)- 2-{2-[4-(ethoxycarbonyl)-1H- pyrazol-1-yl]ethyl} piperazine-1,4-dicarboxylate | K₂CO₃ | 487 |
| 360 | 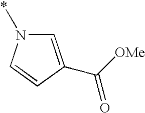 | 1-tert-Butyl 4-benzyl (2R)- 2-{2-[3-(methoxycarbonyl)- 1H-pyrrol-1-yl]ethyl} piperazine-1,4-dicarboxylate | NaH | 472 |
| 361 | 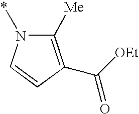 | 1-tert-Butyl 4-benzyl (2R)- 2-{2-[3-(ethoxycarbonyl)-2- methyl-1H-pyrrol-1-yl]ethyl} piperazine-1,4-dicarboxylate | NaH | 500 |
| 362 | 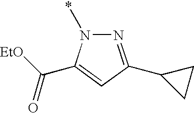 | 1-tert-Butyl 4-benzyl (2R)- 2-{2-[3-cyclopropyl-5-(ethoxy- carbonyl)-1H-pyrazol-1-yl]ethyl} piperazine-1,4-dicarboxylate | K₂CO₃ | 527 |
| 363 | 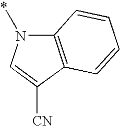 | 1-tert-Butyl 4-benzyl (2R)- 2-[2-(3-cyano-1H-1ndol-1-yl)ethyl] piperazine-1,4-dicarboxylate | NaH | 433** |

### Reference Example 364

### 1-tert-Butyl 4-benzyl (2R)-2-[2-(3-oxo-2,3-dihydro-1H-indazol-1-yl)ethyl]piperazine-1,4-dicarboxylate

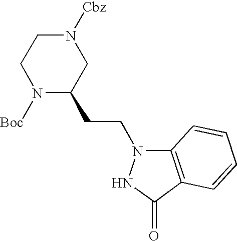

A mixture of 1-tert-butyl 4-benzyl (2R)-2-{2-[(methylsulfonyl)oxy]ethyl}piperazine-1,4-dicarboxylate (700 mg), 1,2-dihydro-3H-indazol-3-one (212 mg), potassium carbonate (450 mg) and DMF (6 ml) was stirred at 80° C. for 3 hr, the insoluble material was filtered off using silica gel, and the filtrate was concentrated under reduced pressure. The residue was subjected to silica gel column chromatography, and the fraction eluted with ethyl acetate-hexane (1:9-1:0) was concentrated under reduced pressure to give the object compound (423 mg).

¹H-NMR (CDCl₃) δ 1.36 (9H, s), 2.05 (1H, s), 2.19 (1H, br s), 2.89 (1H, br s), 3.08 (2H, br s), 4.05-4.16 (1H, m), 4.12 (1H, d), 4.41 (2H, br s), 5.14 (2H, s), 7.07 (1H, td), 7.25-7.39 (9H, m), 7.65 (1H, br s)

MS (ESI+, m/e) 481 (M+1)

In the same manner as in Reference Example 364, the following compounds (Reference Examples 365-371) were obtained.

### Reference Example 365

### 1-tert-Butyl 4-benzyl (2R)-2-[2-(2-oxo-2,3-dihydro-1H-benzimidazol-1-yl)ethyl]piperazine-1,4-dicarboxylate

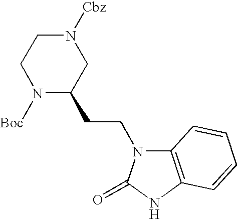

¹H-NMR (CDCl₃) δ 1.35 (9H, br s), 1.98 (4H, br s), 2.90 (1H, br s), 3.04 (2H, br s), 3.84 (1H, br s), 3.96 (1H, br s), 4.14 (2H, br s), 5.14 (2H, br s), 7.03 (3H, br s), 7.29 (6H, br s), 9.17 (1H, br s)

MS (ESI+, m/e) 481 (M+1)

### Reference Example 366

### 1-tert-Butyl 4-benzyl (2R)-2-[2-(2-oxo-1,3-benzoxazol-3(2H)-yl)ethyl]piperazine-1,4-dicarboxylate

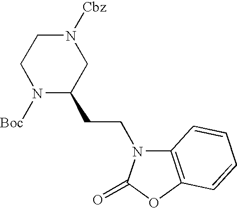

¹H-NMR (CDCl₃) δ 1.38 (9H, s), 1.95 (2H, br s), 3.03 (2H, br s), 3.81 (2H, br s), 3.95 (1H, br s), 4.04-4.19 (1H, m), 4.12 (2H, d), 5.14 (2H, q), 7.05 (1H, s), 7.17 (3H, ddd), 7.11-7.22 (1H, m), 7.25-7.35 (5H, m)

MS (ESI+, m/e) 482 (M+1)

### Reference Example 367

### 1-tert-Butyl 4-benzyl (2R)-2-[2-(3-oxo-2,3-dihydro-4H-1,4-benzoxazin-4-yl)ethyl]piperazine-1,4-dicarboxylate

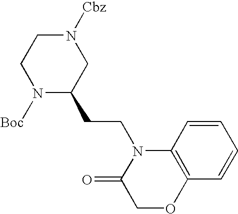

¹H-NMR (CDCl₃) δ 1.44 (9H, br s), 1.88 (2H, br s), 2.90 (1H, br s), 3.05 (2H, br s), 3.82 (2H, br s), 4.09 (4H, br s), 4.60 (2H, br s), 5.14 (2H, br s), 6.96 (4H, br s), 7.31 (5H, br s)

MS (ESI+, m/e) 496 (M+1)

### Reference Example 368

### 1-tert-Butyl 4-benzyl (2R)-2-[2-(3-(cyclopent-1-en-1-yl)-2-oxo-2,3-dihydro-1H-benzimidazol-1-yl)ethyl]piperazine-1,4-dicarboxylate

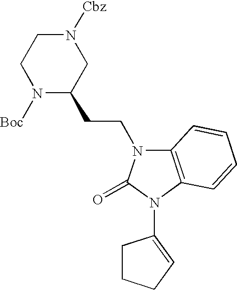

¹H-NMR (CDCl₃) δ 1.36 (9H, br s), 1.89 (1H, d), 2.08 (1H, qd), 1.98-2.13 (3H, m), 2.56 (2H, td), 2.81-2.97 (3H, m), 3.00 (1H, d), 3.09 (2H, br s), 3.83 (1H, d), 3.94 (1H, br s), 4.21 (2H, br s), 5.13 (2H, q), 5.92 (1H, t), 6.91 (1H, br s), 7.03-7.12 (2H, m), 7.13-7.20 (1H, m), 7.22-7.36 (4H, m), 7.28 (1H, d)

MS (ESI+, m/e) 547 (M+1)

### Reference Example 369

### 1-tert-Butyl 4-benzyl (2R)-2-[2-(3-(cyclopent-1-en-1-yl)-2-oxo-2,3-dihydro-1H-benzimidazol-1-yl)ethyl]piperazine-1,4-dicarboxylate

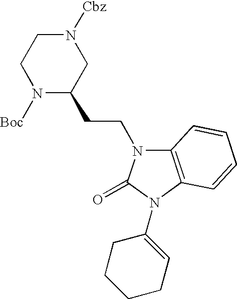

¹H-NMR (CDCl₃) δ 1.38 (9H, br s), 1.69-1.79 (2H, m), 1.80-1.93 (3H, m), 2.25-2.41 (4H, m), 2.28 (3H, d), 2.89 (1H, br s), 3.04 (2H, br s), 3.84 (1H, d), 3.94 (1H, br s), 4.11 (2H, br s), 5.13 (2H, q), 5.91 (1H, br s), 6.95-7.09 (4H, m), 7.22-7.37 (5H, m)

MS (ESI+, m/e) 561 (M+1)

### Reference Example 370

### 1-tert-Butyl 4-benzyl (2R)-2-{2-[4-(ethoxycarbonyl)-2H-1,2,3-triazol-2-yl]ethyl}piperazine-1,4-dicarboxylate

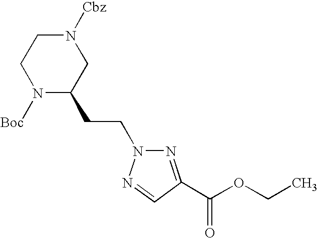

MS (ESI+, m/e) 488 (M+1)

### Reference Example 371

### 1-tert-Butyl 4-benzyl (2R)-2-{2-[5-(ethoxycarbonyl)-1H-1,2,3-triazol-1-yl]ethyl}piperazine-1,4-dicarboxylate

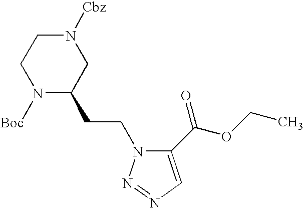

MS (ESI+, m/e) 488 (M+1)

### Reference Example 372

### 1-tert-Butyl 4-benzyl (2R)-2-[2-(3-methyl-2-oxo-2,3-dihydro-1H-benzimidazol-1-yl)ethyl]piperazine-1,4-dicarboxylate

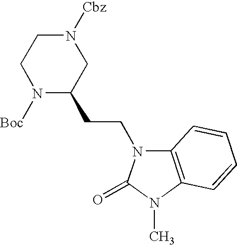

A mixture of 1-tert-butyl 4-benzyl (2R)-2-[2-(2-oxo-2,3-dihydro-1H-benzimidazol-1-yl)ethyl]piperazine-1,4-dicarboxylate (515 mg), methyl iodide (100 μl), cesium carbonate (1.00 g) and DMA (5 ml) was stirred at room temperature for 3 hr, and poured into water, and the mixture was extracted with ethyl acetate. The extract was washed with saturated brine, and dried over anhydrous magnesium sulfate, and the solvent was evaporated under reduced pressure. The residue was subjected to silica gel column chromatography, and the fraction eluted with ethyl acetate-hexane (1:9-1:0) was concentrated under reduced pressure to give the object compound (473 mg).

¹H-NMR (CDCl₃) δ 1.30 (9H, br s), 1.85-2.01 (2H, m), 2.93 (1H, d), 3.01 (2H, br s), 3.34-3.45 (3H, m), 3.81 (2H, br s), 3.94 (1H, br s), 4.04-4.20 (1H, m), 4.12 (2H, q), 5.05-5.20 (2H, m), 6.87-7.02 (2H, m), 7.03-7.14 (1H, m), 7.03-7.14 (1H, m), 7.22-7.36 (5H, m)

MS (ESI+, m/e) 495 (M+1)

### Reference Example 373

### 1-tert-Butyl 4-benzyl (2R)-2-(2-azidoethyl)piperazine-1,4-dicarboxylate

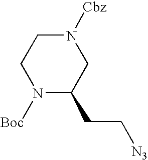

A mixture of 1-tert-butyl 4-benzyl (2R)-2-{2-[(methylsulfonyl)oxy]ethyl}piperazine-1,4-dicarboxylate (3.00 g), sodium azide (2.50 g) and DMF (20 ml) was stirred at 80° C. for 12 hr, and poured into water, and the mixture was extracted with ethyl acetate. The extract was washed with saturated brine, and dried over anhydrous magnesium sulfate, and the solvent was evaporated under reduced pressure. The residue was subjected to silica gel column chromatography, and the fraction eluted with ethyl acetate-hexane (1:19-4:1) was concentrated under reduced pressure to give the object compound (2.19 g).

¹H-NMR (CDCl₃) δ 1.47 (9H, s), 1.69 (1H, br s), 1.84 (1H, t), 1.84 (1H, d), 2.93 (1H, d), 2.92 (1H, d), 3.01 (1H, br s), 3.25 (2H, br s), 4.00 (2H, br s), 4.27 (1H, br s), 5.14 (2H, d), 7.30-7.40 (5H, m)

MS (ESI+, m/e) 390 (M+1)

### Reference Example 374

### 1-tert-Butyl 4-benzyl (2R)-2-{2-[4-(hydroxymethyl)-1H-1,2,3-triazol-1-yl]ethyl}piperazine-1,4-dicarboxylate

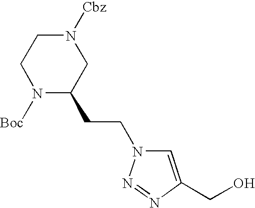

A mixture of 1-tert-butyl 4-benzyl (2R)-2-(2-azidoethyl)piperazine-1,4-dicarboxylate (500 mg), propargyl alcohol (360 mg) and toluene (7 ml) was stirred at 130° C. for 12 hr in a sealed stainless tube, and the solvent was evaporated under reduced pressure. The residue was subjected to silica gel column chromatography, and the fraction eluted with ethyl acetate-hexane (1:19-4:1) was concentrated under reduced pressure to give the object compound (510 mg).

¹H-NMR (CDCl₃) δ 1.46 (9H, d), 1.77-1.94 (1H, m), 2.04 (1H, br s), 2.18 (1H, d), 2.95 (2H, br s), 3.26 (1H, br s), 3.86 (1H, br s), 4.02 (2H, br s), 4.13 (1H, br s), 4.27 (2H, br s), 4.64 (1H, br s), 4.78 (1H, s), 5.14 (2H, d), 7.09-7.21 (1H, m), 7.23-7.38 (5H, m)

MS (ESI+, m/e) 446 (M+1)

In the same manner as in Reference Example 374, the following compounds (Reference Examples 375-378) were obtained.

### Reference Example 375

### 1-tert-Butyl 4-benzyl (2R)-2-{2-[4-(2-hydroxyethyl)-1H-1,2,3-triazol-1-yl]ethyl}piperazine-1,4-dicarboxylate

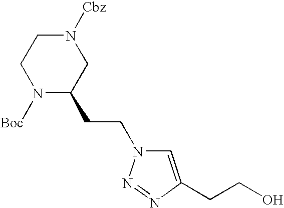

¹H-NMR (CDCl₃) δ 1.45 (9H, br s), 1.79-1.95 (1H, m), 2.03-2.19 (2H, m), 2.34 (1H, br s), 2.50 (1H, br s), 2.90 (4H, br s), 3.27 (1H, br s), 3.74 (1H, br s), 3.85 (1H, br s), 3.95 (2H, br s), 4.06 (1H, br s), 4.28 (1H, br s), 5.14 (2H, br s), 7.16 (1H, br s), 7.26 (1H, br s), 7.35 (4H, br s)

MS (ESI+, m/e) 460 (M+1)

### Reference Example 376

### 1-tert-Butyl 4-benzyl (2R)-2-[2-(4-cyclopropyl-1H-1,2,3-triazol-1-yl)ethyl]piperazine-1,4-dicarboxylate

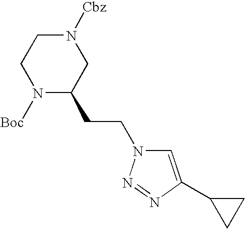

¹H-NMR (CDCl₃) δ 0.67 (1H, br s), 0.84 (2H, dd), 0.89-1.04 (1H, m), 0.94 (2H, td), 1.43 (9H, d), 1.94 (1H, dt), 2.14 (1H, br s), 2.35 (1H, s), 2.87 (1H, br s), 3.03 (1H, br s), 4.12 (2H, d), 4.08 (1H, br s), 4.25 (2H, br s), 5.13 (2H, d), 7.15-7.19 (1H, m), 7.22-7.37 (5H, m)

MS (ESI+, m/e) 456 (M+1)

### Reference Example 377

### 1-tert-Butyl 4-benzyl (2R)-2-{2-[4-(ethoxycarbonyl)-1H-1,2,3-triazol-1-yl]ethyl}piperazine-1,4-dicarboxylate

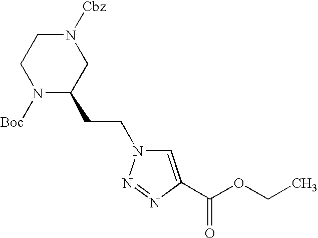

MS (ESI+, m/e) 488 (M+1)

### Reference Example 378

### 1-tert-Butyl 4-benzyl (2R)-2-[2-(4-acetyl-1H-1,2,3-triazol-1-yl)ethyl]piperazine-1,4-dicarboxylate

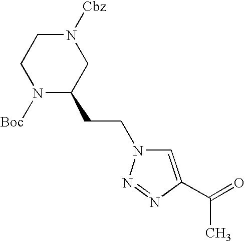

¹H-NMR (CDCl₃) δ 1.44 (9H, s), 1.98-2.08 (1H, m), 2.25 (1H, d), 2.69 (3H, s), 2.92 (2H, d), 3.03 (1H, br s), 3.94 (1H, br s), 3.98-4.21 (3H, m), 4.38 (2H, br s), 5.06-5.21 (1H, m), 5.14 (1H, d), 7.34 (5H, s), 8.15 (1H, s)

MS (ESI+, m/e) 458 (M+1)

### Reference Example 379

### 1-tert-Butyl 4-benzyl (2R)-2-(2-{4-[(acetyloxy)methyl]-1H-1,2,3-triazol-1-yl}ethyl)piperazine-1,4-dicarboxylate

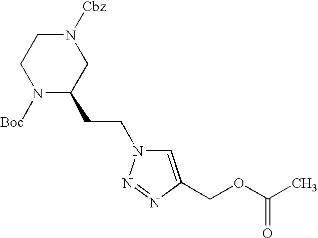

A mixture of 1-tert-butyl 4-benzyl (2R)-2-{2-[4-(hydroxymethyl)-1H-1,2,3-triazol-1-yl]ethyl}piperazine-1,4-dicarboxylate (360 mg), acetic anhydride (1.0 ml) and pyridine (1.0 ml) was stirred at room temperature for 12 hr, and concentrated under reduced pressure to give the object compound (390 mg).

¹H-NMR (CDCl₃) δ 1.44 (9H, s), 2.03-2.16 (4H, m), 2.23 (3H, s), 2.89 (1H, br s), 2.96 (1H, br s), 3.04 (1H, br s), 4.15 (1H, br s), 4.21-4.36 (3H, m), 5.07-5.22 (4H, m), 7.30-7.40 (5H, m), 7.55-7.72 (1H, m)

MS (ESI+, m/e) 488 (M+1)

### Reference Example 380

### 1-tert-Butyl 4-benzyl (2R)-2-[2-(1H-indazol-1-yl)ethyl]piperazine-1,4-dicarboxylate and 1-tert-butyl 4-benzyl (2R)-2-[2-(2H-indazol-2-yl)ethyl]piperazine-1,4-dicarboxylate

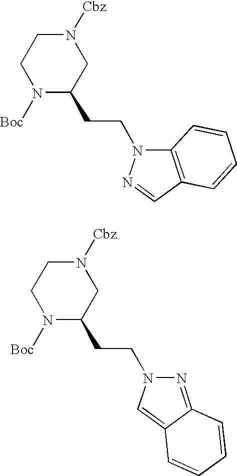

A mixture of 1-tert-butyl 4-benzyl (2R)-2-{2-[(methylsulfonyl)oxy]ethyl}piperazine-1,4-dicarboxylate (620 mg), 1H-indazole (331 mg), potassium carbonate (1.2 g) and DMF (7 ml) was stirred at 50° C. for 10 hr, and poured into water, and the mixture was extracted with ethyl acetate. The extract was washed with saturated brine, and dried over anhydrous sodium sulfate, and the solvent was evaporated under reduced pressure. The residue was subjected to silica gel column chromatography, and the fractions eluted with ethyl acetate-hexane (1:1) were concentrated under reduced pressure, respectively. The residue of the less polar fraction was vacuum-dried to give 1-tert-butyl 4-benzyl (2R)-2-[2-(1H-indazol-1-yl)ethyl]piperazine-1,4-dicarboxylate (380 mg), and the residue of the more polar fraction was vacuum-dried to give 1-tert-butyl 4-benzyl (2R)-2-[2-(2H-indazol-2-yl)ethyl]piperazine-1,4-dicarboxylate (170 mg), as an amorphous solid, respectively.

MS (ESI+, m/e) 465 (M+1)

MS (ESI+, m/e) 465 (M+1)

### Reference Example 381

### 1-tert-Butyl 4-benzyl (2R)-2-{2-[5-(ethoxycarbonyl)-3-methyl-1H-pyrazol-1-yl]ethyl}piperazine-1,4-dicarboxylate and 1-tert-butyl 4-benzyl (2R)-2-{2-[3-(ethoxycarbonyl)-5-methyl-1H-pyrazol-1-yl]ethyl}piperazine-1,4-dicarboxylate

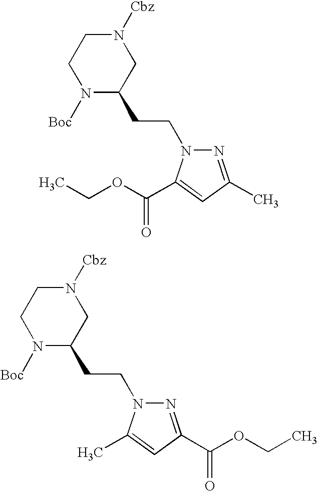

A mixture of 1-tert-butyl 4-benzyl (2R)-2-{2-[(methylsulfonyl)oxy]ethyl}piperazine-1,4-dicarboxylate (800 mg), ethyl 5-methyl-1H-pyrazole-3-carboxylate (560 mg), potassium carbonate (1.1 g) and DMF (20 ml) was stirred at 50° C. for 10 hr, and poured into water, and the mixture was extracted with ethyl acetate. The extract was washed with saturated brine, and dried over anhydrous sodium sulfate, and the solvent was evaporated under reduced pressure. The residue was subjected to silica gel column chromatography, and the fractions eluted with ethyl acetate-hexane (1:1) were concentrated under reduced pressure, respectively. The residue of the less polar fraction was vacuum-dried to give 1-tert-butyl 4-benzyl (2R)-2-{2-[3-(ethoxycarbonyl)-5-methyl-1H-pyrazol-1-yl]ethyl}piperazine-1,4-dicarboxylate (470 mg), and the residue of the more polar fraction was vacuum-dried to give 1-tert-butyl 4-benzyl (2R)-2-{2-[5-(ethoxycarbonyl)-3-methyl-1H-pyrazol-1-yl]ethyl}piperazine-1,4-dicarboxylate (390 mg), as an amorphous solid, respectively.

MS (ESI+, m/e) 501 (M+1)

MS (ESI+, m/e) 501 (M+1)

In the same manner as in Reference Example 381, the following compound (Reference Example 382) was obtained.

### Reference Example 382

### 1-tert-Butyl 4-benzyl (2R)-2-{2-[3-(methoxycarbonyl)-1H-indazol-1-yl]ethyl}piperazine-1,4-dicarboxylate and 1-tert-butyl 4-benzyl (2R)-2-{2-[3-(methoxycarbonyl)-2H-indazol-2-yl]ethyl}piperazine-1,4-dicarboxylate

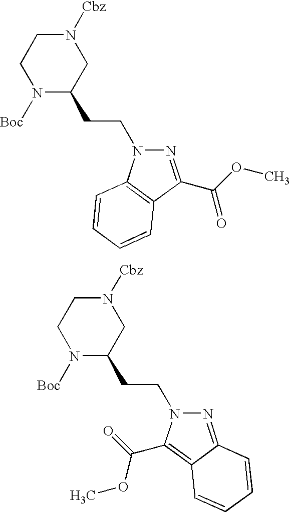

MS (ESI+, m/e) 523 (M+1)

MS (ESI+, m/e) 523 (M+1)

### Reference Example 383

### Benzyl (3R)-3-(2-phenoxyethyl)piperazine-1-carboxylate

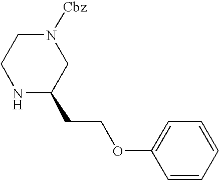

1-tert-Butyl 4-benzyl (2R)-2-(2-phenoxyethyl)piperazine-1,4-dicarboxylate (585 mg) was dissolved in dichloromethane (2 ml), TFA (4 ml) was added thereto, and the mixture was stirred at room temperature for 50 min. The reaction mixture was poured into saturated aqueous sodium hydrogen carbonate-saturated brine (1:1) by small portions. To the mixture was added potassium carbonate by small portions to basify the mixture, and the mixture was extracted with ethyl acetate. The extract was washed with saturated brine, and dried over anhydrous magnesium sulfate, and the solvent was evaporated under reduced pressure to give the object compound (435 mg) as an oil.

MS (ESI+, m/e) 341 (M+1)

In the same manner as in Reference Example 383, the following compounds (Reference Examples 384-422) shown in Table 6-1-Table 6-5 were obtained.

TABLE 6-1

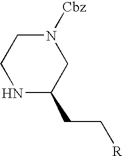

| Ref. Ex. No. | R | Compound | MS(ESI+) |
|---|---|---|---|
| 384 | 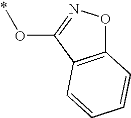 | Benzyl (3R)-3-[2-(1,2- benzisoxazol-3-yloxy)ethyl] piperazine-1-carboxylate | 382 |
| 385 | 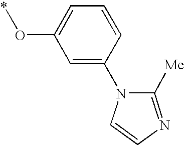 | Benzyl (3R)-3-{2-[3-(2-methyl- 1H-imidazol-1-yl)phenoxy]ethyl} piperazine-1-carboxylate | 421 |
| 386 | 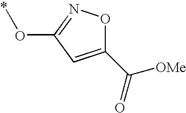 | Benzyl (3R)-3-(2-{[5-(methoxy carbonyl)-isoxazol-3-yl]oxy} ethyl)piperazine-1-carboxylate | 390 |
| 387 | 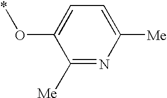 | Benzyl (3R)-3-{2-[(2,6- dimethylpyridin-3-yl)oxy]ethyl} piperazine-1-carboxylate | 370 |
| 388 | 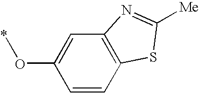 | Benzyl (3R)-3-{2-[(2-methyl-1,3- benzothiazol-5-yl)oxy]ethyl} piperazine-1-carboxylate | 412 |
| 389 | 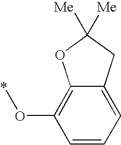 | Benzyl (3R)-3-{2-[(2,2-dimethyl- 2,3-dihydro-1-benzofuran-7-yl)-oxy] ethyl}piperazine-1-carboxylate | 411 |
| 390 | 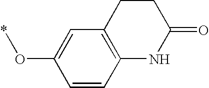 | Benzyl (3R)-3-{2-[(2-oxo-1,2,3,4- tetrahydroquinolin-6-yl)oxy]ethyl} piperazine-1-carboxylate | 410 |
| 391 | 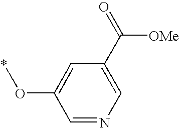 | Benzyl (3R)-3-(2-{[5-(methoxy carbonyl)pyridin-3-yl]oxy} ethyl)piperazine-1-carboxylate | 400 |
| 392 | 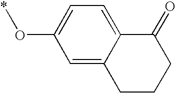 | Benzyl (3R)-3-{2-[(5-oxo-5,6,7,8- tetrahydronaphthalen-2-yl)oxy] ethyl}piperazine-1-carboxylate | 409 |
| 393 | 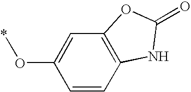 | Benzyl (3R)-3-{2-[(2-oxo-2,3- dihydro-1,3-benzoxazol-6-yl)oxy] ethyl}piperazine-1-carboxylate | 398 |

TABLE 6-2

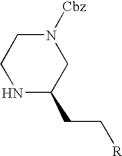

| Ref. Ex. No. | R | Compound | MS(ESI+) |
|---|---|---|---|
| 394 | 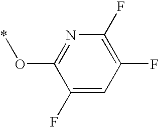 | Benzyl (3R)-3-{2-[(3,5,6- trifluoropyridin-2-yl)oxy] ethyl}piperazine-1-carboxylate | 396 |
| 395 | 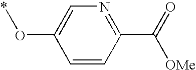 | Benzyl (3R)-3-(2-{[6- (methoxycarbonyl)pyridin-3-yl] oxy}ethyl)piperazine-1-carboxylate | 400 |
| 396 | 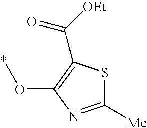 | Benzyl (3R)-3-(2-{[5-(ethoxy- carbonyl)-2-methyl-1,3-thiazol-4-yl] oxy}ethyl)piperazine-1-carboxylate | 434 |
| 397 | 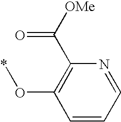 | Benzyl (3R)-3-(2-{[2-(methoxy carbonyl)pyridin-3-yl]oxy}ethyl) piperazine-1-carboxylate | 400 |
| 398 | 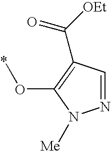 | Benzyl (3R)-3-(2-{[4-(ethoxy- carbonyl)-1-methyl-1H-pyrazol-5-yl] oxy}ethyl)piperazine-1-carboxylate | 417 |
| 399 | 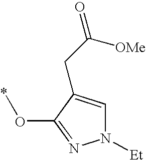 | Benzyl (3R)-3-(2-{[1-ethyl-4-(2- methoxy-2-oxoethyl)-1H-pyrazol-3-yl] oxy}ethyl)piperazine-1-carboxylate | 431 |
| 400 | 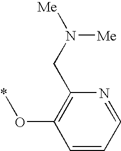 | Benzyl (3R)-3-[2-({2-[(dimethyl- amino)methyl]pyridin-3-yl}oxy) ethyl]piperazine-1-carboxylate | 399 |
| 401 | 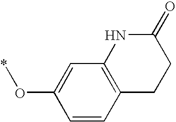 | Benzyl (3R)-3-{2-[(2-oxo-1,2,3,4- tetrahydroquinolin-7-yl)oxy] ethyl}piperazine-1-carboxylate | 410 |
| 402 | 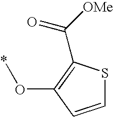 | Benzyl (3R)-3-(2-{[2- (methoxycarbonyl)-3-thienyl]oxy} ethyl)piperazine-1-carboxylate | 405 |

TABLE 6-3

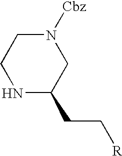

| Ref. Ex. No. | R | Compound | MS(ESI+) |
|---|---|---|---|
| 403 | 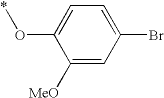 | Benzyl (3R)-3-[2-(4-bromo-2- methoxyphenoxy)ethyl]piperazine-1- carboxylate | 449 |
| 404 | 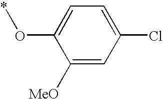 | Benzyl (3R)-3-[2-(4-chloro-2- methoxyphenoxy)ethyl]piperazine-1- carboxylate | 405 |
| 405 | 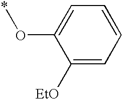 | Benzyl (3R)-3-[2-(2-ethoxyphenoxy) ethyl]piperazine-1-carboxylate | 385 |
| 406 | 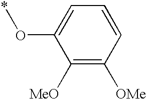 | Benzyl (3R)-3-[2-(2,3-dimethoxy- phenoxy)ethyl]piperazine- 1-carboxylate | 401 |
| 407 | 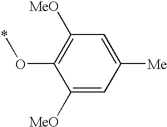 | Benzyl (3R)-3-[2-(2,6-dimethoxy-4- methylphenoxy)ethyl]piperazine-1- carboxylate | 415 |
| 408 | 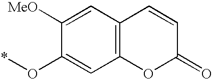 | Benzyl (3R)-3-{2-[(6-methoxy-2- oxo-2H-chromen-7-yl)oxy]ethyl} piperazine-1-carboxylate | 439 |
| 409 | 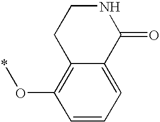 | Benzyl (3R)-3-{2-[(1-oxo-1,2,3,4- tetrahydroisoquinolin-5-yl)oxy] ethyl}piperazine-1-carboxylate | 410 |
| 410 | 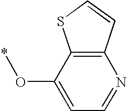 | Benzyl (3R)-3-[2-(thieno[3,2- b]pyridin-7-yloxy)ethyl]piperazine- 1-carboxylate | 398 |
| 411 | 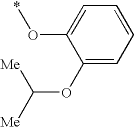 | Benzyl (3R)-3-[2-(2-isopropoxy- phenoxy)ethyl]piperazine- 1-carboxylate | 399 |

TABLE 6-4

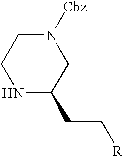

| Ref. Ex. No. | R | Compound | MS(ESI+) |
|---|---|---|---|
| 412 | 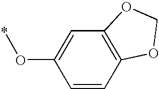 | Benzyl (3R)-3-[2-(1-3-benzodioxol- 5-yloxy)ethyl]piperazine-1-carboxylate | 385 |
| 413 | 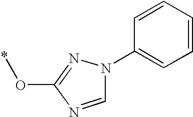 | Benzyl (3R)-3-{2-[(1-phenyl-1H- 1,2,4-triazol-3-yl)oxy]ethyl} piperazine-1-carboxylate | 408 |
| 414 | 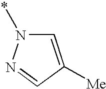 | Benzyl (3R)-3-[2-(4-methyl-1H- pyrazol-1-yl)ethyl]piperazine-1- carboxylate | 329 |
| 415 | 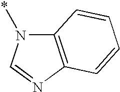 | Benzyl (3R)-3-[2-(1H-benzimidazol- 1-yl)ethyl]piperazine-1-carboxylate | 365 |
| 416 | 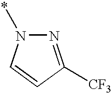 | Benzyl (3R)-3-{2-[3- (trifluoromethyl)-1H-pyrazol-1- yl]ethyl}piperazine-1-carboxylate | 383 |
| 417 | 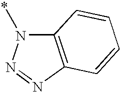 | Benzyl (3R)-3-[2-(1H-benzotriazol- 1-yl) ethyl]piperazine-1-carboxylate | 366 |
| 418 | 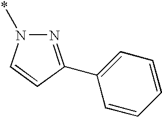 | Benzyl (3R)-3-[2-(3-phenyl-1H- pyrazol-1-yl)ethyl]piperazine-1- carboxylate | 391 |
| 419 | 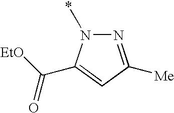 | Benzyl (3R)-3-{2-[5- (ethoxycarbonyl)-3-methyl-1H- pyrazol-1-yl]ethyl}piperazine- 1-carboxylate | 401 |
| 420 | 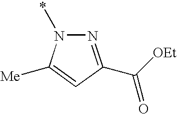 | Benzyl (3R)-3-{2-[3- (ethoxycarbonyl)-5-methyl-1H- pyrazol-1-yl]ethyl}piperazine- 1-carboxylate | 401 |
| 421 | 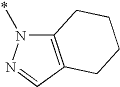 | Benzyl (3R)-3-[2-(4,5,6,7- tetrahydro-1H-indazol-1- yl)ethyl]piperazine-1-carboxylate | 369 |

TABLE 6-5

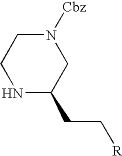

| Ref. | | | |
|---|---|---|---|
| Ex. No. | R | Compound | MS(ESI+) |
| 422 | 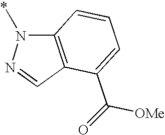 | Benzyl (3R)-3-{2-[4- (methoxycarbonyl)- 1H-indazol-1-yl]ethyl) piperazine-1- carboxylate | 423 |

### Reference Example 423

### Benzyl (3R)-3-[2-(1H-indazol-1-yl)ethyl]piperazine-1-carboxylate

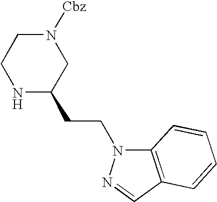

1-tert-Butyl 4-benzyl (2R)-2-[2-(1H-indazol-1-yl)ethyl]piperazine-1,4-dicarboxylate (380 mg) was dissolved in chloroform (5 ml), TFA (5 ml) was added, and the mixture was stirred at room temperature for 1 hr. The reaction mixture was diluted with toluene (10 ml), and the solvent was evaporated under reduced pressure. The residue was dissolved in ethyl acetate, and the solution was washed with saturated aqueous sodium hydrogen carbonate, and dried over anhydrous sodium sulfate. The solvent was evaporated under reduced pressure to give the object compound (300 mg) as an oil.

MS (ESI+, m/e) 365 (M+1)

In the same manner as in Reference Example 423, the following compound (Reference Example 424) was obtained.

### Reference Example 424

### Benzyl (3R)-3-[2-(2H-indazol-2-yl)ethyl]piperazine-1-carboxylate

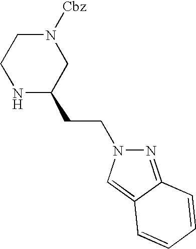

MS (ESI+, m/e) 365 (M+1)

### Reference Example 425

### Benzyl (3R)-3-[2-(3-oxo-2,3-dihydro-1H-indazol-1-yl)ethyl]piperazine-1-carboxylate hydrochloride

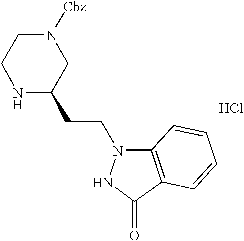

A mixture of 1-tert-butyl 4-benzyl (2R)-2-[2-(3-oxo-2,3-dihydro-1H-indazol-1-yl)ethyl]piperazine-1,4-dicarboxylate (415 mg) and 2N hydrogen chloride-ethyl acetate solution was stirred at room temperature for 4 hr, and concentrated under reduced pressure to give the object compound (325 mg).

MS (ESI+, m/e) 381 (M+1)

In the same manner as in Reference Example 425, the following compounds (Reference Examples 426-502) shown in Table 7-1-Table 7-8 were obtained.

TABLE 7-1

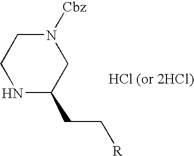

| Ref. Ex. No. | R | Compound | MS(ESI+) |
|---|---|---|---|
| 426 | 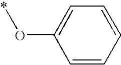 | Benzyl (3R)-3-(2-phenoxyethyl) piperazine-1-carboxylate hydrochloride | 341 |
| 427 | 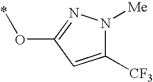 | Benzyl (3R)-3-(2-{[1-methyl-5- (trifluoromethyl)-1H-pyrazol-3- yl]oxy}ethyl)piperazine-1- carboxylate hydrochloride | 413 |
| 428 | 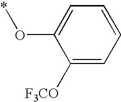 | Benzyl (3R)-3-{2-[2-(trifluoro methoxy)phenoxy]ethyl}piperazine- 1-carboxylate hydrochloride | 425 |
| 429 | 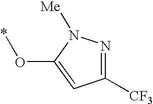 | Benzyl (3R)-3-(2-{[1-methyl-3- (trifluoromethyl)-1H-pyrazol-5- yl]oxy}ethyl)piperazine-1- carboxylate hydrochloride | 413 |
| 430 | 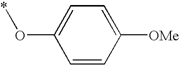 | Benzyl (3R)-3-[2-(4- methoxyphenoxy)ethyl]piperazine-1- carboxylate hydrochloride | 371 |
| 431 | 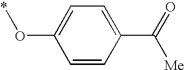 | Benzyl (3R)-3-[2-(4- acetylphenoxy)ethyl]piperazine-1- carboxylate hydrochloride | 383 |
| 432 | 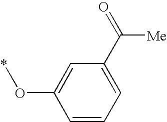 | Benzyl (3R)-3-[2-(3- acetylphenoxy)ethyl]piperazine-1- carboxylate hydrochloride | 383 |
| 433 | 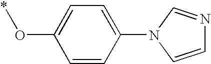 | Benzyl (3R)-3-{2-[4-(1H- imidazol-1-yl)phenoxy]ethyl} piperazine-1-carboxylate hydrochloride | 407 |
| 434 | 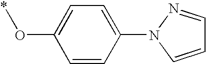 | Benzyl (3R)-3-{2-[4- (1H-pyrazol-1-yl)phenoxy]ethyl} piperazine-1-carboxylate hydrochloride | 407 |

TABLE 7-2

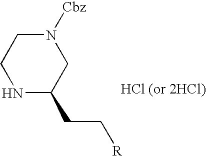

| Ref. Ex. No. | R | Compound | MS(ESI+) |
|---|---|---|---|
| 435 | 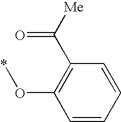 | Benzyl (3R)-3-[2-(2- acetylphenoxy)ethyl]piperazine- 1-carboxylate hydrochloride | 383 |
| 436 | 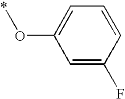 | Benzyl (3R)-3-[2-(3- fluorophenoxy)ethyl]piperazine- 2-carboxylate hydrochloride | 359 |
| 437 | 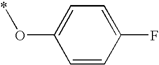 | Benzyl (3R)-3-[2-(4- fluorophenoxy)ethyl]piperazine- 3-carboxylate hydrochloride | 359 |
| 438 | 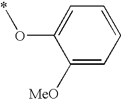 | Benzyl (3R)-3-[2-(2- methoxyphenoxy)ethyl]piperazine- 1-carboxylate hydrochloride | 371 |
| 439 | 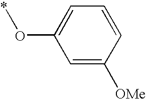 | Benzyl (3R)-3-[2-(3- methoxyphenoxy)ethyl]piperazine- 2-carboxylate hydrochloride | 371 |
| 440 | 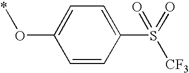 | Benzyl (3R)-3-(2-{4- [(trifluoromethyl)sulfonyl]phenoxy} ethyl)piperazine-1-carboxylate hydrochloride | 473 |
| 441 | 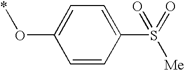 | Benzyl (3R)-3-{2-[4-(methyl sulfonyl)phenoxy]ethyl}piperazine- 1-carboxylate hydrochloride | 419 |
| 442 | 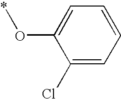 | Benzyl (3R)-3-[2-(2- chlorophenoxy)ethyl]piperazine-1- carboxylate hydrochloride | 375 |
| 443 | 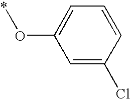 | Benzyl (3R)-3-[2-(3- chlorophenoxy)ethyl]piperazine- 2-carboxylate hydrochloride | 375 |
| 444 | 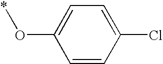 | Benzyl (3R)-3-[2-(4- chlorophenoxy)ethyl]piperazine-3- carboxylate hydrochloride | 375 |

TABLE 7-3

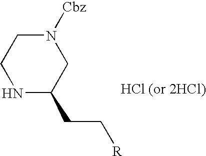

| Ref. Ex. No. | R | Compound | MS(ESI+) |
|---|---|---|---|
| 445 | 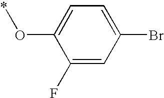 | Benzyl (3R)-3-[2-(4-bromo-2- fluorophenoxy)ethyl]piperazine-1- carboxylate hydrochloride | 438 |
| 446 | 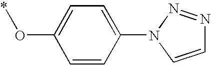 | Benzyl (3R)-3-{2-[4-(1H-1,2,3- triazol-1-yl)phenoxy]ethyl} piperazine-1-carboxylate hydrochloride | 408 |
| 447 | 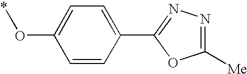 | Benzyl (3R)-3-{2-[4-(5-methyl- 1,3,4-oxadiazol-2-yl)phenoxy]ethyl} piperazine-1-carboxylate hydrochloride | 423 |
| 448 | 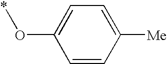 | Benzyl (3R)-3-[2-(4- methylphenoxy)ethyl]piperazine-1- carboxylate hydrochloride | 355 |
| 449 | 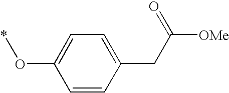 | Benzyl (3R)-3-{2-[4-(2- methoxy-2-oxoethyl)phenoxy)ethyl} piperazine-1-carboxylate hydrochloride | 413 |
| 450 | 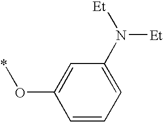 | Benzyl (3R)-3-{2-[3- (diethylamino) phenoxy]ethyl} piperazine-1-carboxylate dihydrochloride | 412 |
| 451 | 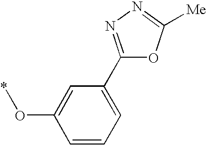 | Benzyl (3R)-3-{2-[3-(5-methyl- 1,3,4-oxadiazol-2-yl)phenoxy]ethyl} piperazine-1-carboxylate hydrochloride | 423 |
| 452 | 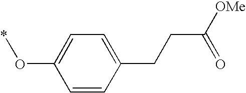 | Benzyl (3R)-3-{2-[4-(3- methoxy-3-oxopropyl)phenoxy]ethyl} piperazine-1-carboxylate hydrochloride | 427 |
| 453 | 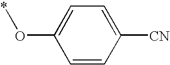 | Benzyl (3R)-3-[2-(4- cyanophenoxy)ethyl]piperazine-1- carboxylate hydrochloride | 366 |
| 454 | 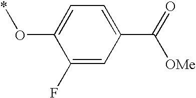 | Benzyl (3R)-3-{2-[2-fluoro-4- (methoxycarbonyl)phenoxy]ethyl} piperazine-1-carboxylate hydrochloride | 417 |

TABLE 7-4

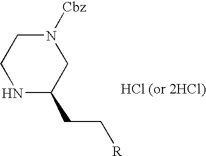

| Ref. Ex. No. | R | Compound | MS(ESI+) |
|---|---|---|---|
| 455 | 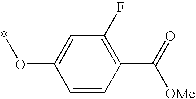 | Benzyl (3R)-3-{2-[3-fluoro-4- (methoxycarbonyl)phenoxy]ethyl} piperazine-1-carboxylate hydrochloride | 417 |
| 456 | 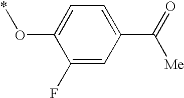 | Benzyl (3R)-3-[2-(4-acetyl-2- fluorophenoxy)ethyl]piperazine-1- carboxylate hydrochloride | 401 |
| 457 | 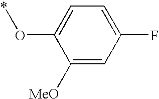 | Benzyl (3R)-3-[2-(4-fluoro-2- methoxyphenoxy)ethyl]piperazine-1- carboxylate hydrochloride | 389 |
| 458 | 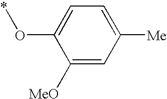 | Benzyl (3R)-3-[2-(2-methoxy-4- methylphenoxy)ethyl]piperazine-1- carboxylate hydrochloride | 385 |
| 459 | 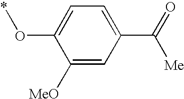 | Benzyl (3R)-3-[2-(4-acetyl-2- methoxyphenoxy)ethyl]piperazine-1- carboxylate hydrochloride | 413 |
| 460 | 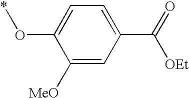 | Benzyl (3R)-3-{2-[4-(ethoxycarbonyl)- 2-methoxyphenoxy)ethyl}piperazine-1- carboxylate hydrochloride | 443 |
| 461 | 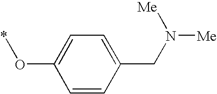 | Benzyl (3R)-3-(2-{4-[(dimethylamino) methyl]phenoxy} ethyl)piperazine- 1-carboxylate dihydrochloride | 398 |
| 462 | 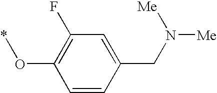 | Benzyl (3R)-3-(2-{4-[(dimethylamino) methyl]-2-fluorophenoxy}ethyl) piperazine-1-carboxylate dihydrochloride | 416 |
| 463 | 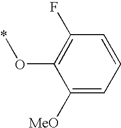 | Benzyl (3R)-3-[2-(2-fluoro-6-methoxy- phenoxy)ethyl]piperazine-1- carboxylate hydrochloride | 389 |
| 464 | 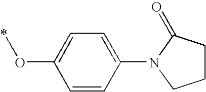 | Benzyl (3R)-3-{2-[4-(2-oxopyrrolidin- 1-yl)phenoxy)ethyl}piperazine-1- carboxylate hydrochloride | 424 |

TABLE 7-5

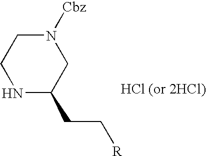

| Ref. Ex. No. | R | Compound | MS(ESI+) |
|---|---|---|---|
| 465 | 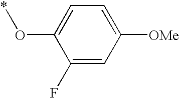 | Benzyl (3R)-3-[2-(2-fluoro-4- methoxyphenoxy)ethyl]piperazine-1- carboxylate hydrochloride | 389 |
| 466 | 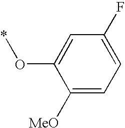 | Benzyl (3R)-3-[2-(5-fluoro-2- methoxyphenoxy)ethyl]piperazine-1- carboxylate hydrochloride | 389 |
| 467 | 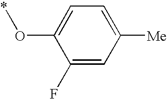 | Benzyl (3R)-3-[2-(2-fluoro-4- methylphenoxy)ethyl]piperazine-1- carboxylate hydrochloride | 373 |
| 468 | 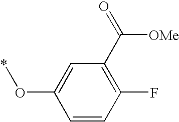 | Benzyl (3R)-3-{2-[4-fluoro-3- (methoxycarbonyl)phenoxy]ethyl} piperazine-1-carboxylate hydrochloride | 417 |
| 469 | 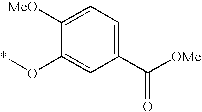 | Benzyl (3R)-3-{2-[2-methoxy-5- (methoxycarbonyl)phenoxy]ethyl} piperazine-1-carboxylate hydrochloride | 429 |
| 470 | 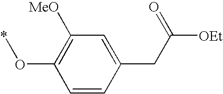 | Benzyl (3R)-3-{2-[4-(2-ethoxy-2- oxoethyl)-2-methoxyphenoxy]ethyl} piperazine-1-carboxylate hydrochloride | 457 |
| 471 | 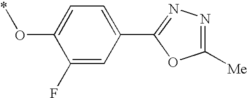 | Benzyl (3R)-3-{2-[2-fluoro-4-(5- methyl-1,3,4-oxadiazol-2-yl)phenoxy] ethyl}piperazine-1- carboxylate hydrochloride | 441 |
| 472 | 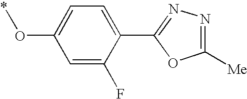 | Benzyl (3R)-3-{2-[3-fluoro-4-(5- methyl-1,3,4-oxadiazol-2-yl)phenoxy] ethyl}piperazine-1- carboxylate hydrochloride | 441 |
| 473 | 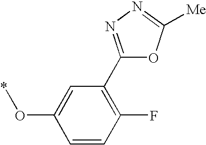 | Benzyl (3R)-3-{2-[4-fluoro-3-(5- methyl-1,3,4-oxadiazol-2-yl)phenoxy] ethyl}piperazine-1- carboxylate hydrochloride | 441 |

TABLE 7-6

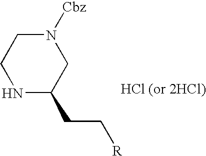

| Ref. Ex. No. | R | Compound | MS(ESI+) |
|---|---|---|---|
| 474 | 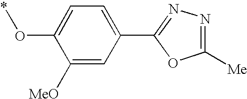 | Benzyl (3R)-3-{2-[2-methoxy-4-(5- methyl-1,3,4-oxadiazol-2-yl)phenoxy] ethyl}piperazine-1- carboxylate hydrochloride | 453 |
| 475 | 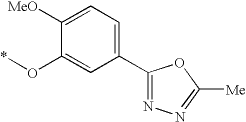 | Benzyl (3R)-3-{2-[2-methoxy-5-(5- methyl-1,3,4-oxadiazol-2-yl)phenoxy] ethyl}piperazine-1- carboxylate hydrochloride | 453 |
| 476 | 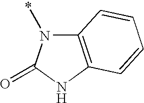 | Benzyl (3R)-3-[2-(2-oxo-2,3- dihydro-1H-benzimidazol-1-yl)ethyl] piperazine-1-carboxylate hydrochloride | 381 |
| 477 | 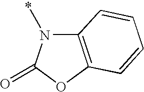 | Benzyl (3R)-3-[2-(2-oxo-1,3- benzoxazol-3(2H)-yl)ethyl]piperazine- 1-carboxylate hydrochloride | 382 |
| 478 | 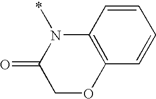 | Benzyl (3R)-3-[2-(3-oxo-2,3-dihydro- 4H-1,4-benzoxazin-4-yl)ethyl] piperazine-1-carboxylate hydrochloride | 396 |
| 479 | 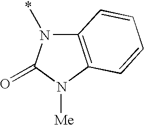 | Benzyl (3R)-3-[2-(3-methyl-2-oxo-2,3- dihydro-1H-benzimidazol-1-yl)ethyl] piperazine-1-carboxylate hydrochloride | 395 |
| 480 | 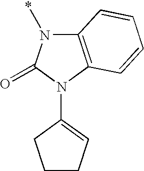 | Benzyl (3R)-3-{2-[3-(cyclopent-1-en-1- yl)-2-oxo-2,3-dihydro-1H- benzimidazol-1-yl]ethyl}piperazine- 1-carboxylate hydrochloride | 447 |
| 481 | 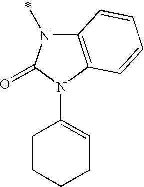 | Benzyl (3R)-3-{2-[3-(cyclohex-1-en-1- yl)-2-oxo-2,3-dihydro-1H- benzimidazol-1-yl]ethyl}piperazine- 1-carboxylate hydrochloride | 461 |
| 482 | 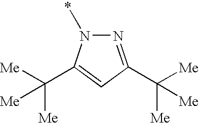 | Benzyl (3R)-3-[2-(3,5-di-tert-butyl-1H- pyrazol-1-yl)ethyl]piperazine-1- carboxylate hydrochloride | 427 |

TABLE 7-7

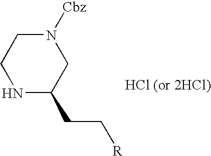

| Ref. Ex. No. | R | Compound | MS(ESI+) |
|---|---|---|---|
| 483 | 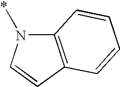 | Benzyl (3R)-3-[2-(1H-indol-1-yl)ethyl] piperazine-1-carboxylate hydrochloride | 364 |
| 484 | 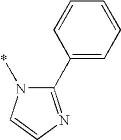 | Benzyl (3R)-3-[2-(2-phenyl-1H- imidazol-1-yl)ethyl]piperazine- 1-carboxylate hydrochloride | 391 |
| 485 | 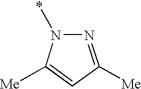 | Benzyl (3R)-3-[2-(3,5-dimethyl-1H- pyrazol-1-yl)ethyl]piperazine-1- carboxylate hydrochloride | 343 |
| 486 | 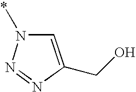 | Benzyl (3R)-3-{2-[4-(hydroxymethyl)- 1H-1,2,3-triazol-1-yl]ethyl} piperazine-1-carboxylate hydrochloride | 346 |
| 487 | 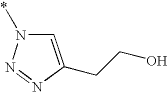 | Benzyl (3R)-3-{2-[4-(2- hydroxyethyl)-1H-1,2,3-triazol-1-yl] ethyl}piperazine-1-carboxylate hydrochloride | 360 |
| 488 | 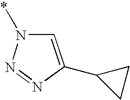 | Benzyl (3R)-3-[2-(4-cyclopropyl-1H- 1,2,3-triazol-1-yl)ethyl]piperazine- 1-carboxylate hydrochloride | 356 |
| 489 | 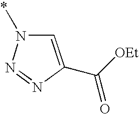 | Benzyl (3R)-3-{2-[4-(ethoxycarbonyl)- 1H-1,2,3-triazol-1-yl]ethyl} piperazine-1-carboxylate hydrochloride | 388 |
| 490 | 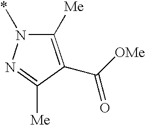 | Benzyl (3R)-3-{2-[4-(methoxy- carbonyl)-3,5-dimethyl-1H-pyrazol-1- yl]ethyl}piperazine-1- carboxylate hydrochloride | 401 |
| 491 | 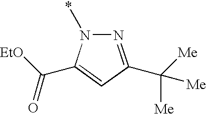 | Benzyl (3R)-3-{2-[3-tert-butyl-5- (ethoxycarbonyl)-1H-pyrazol-1-yl] ethyl}piperazine-1-carboxylate hydrochloride | 443 |
| 492 | 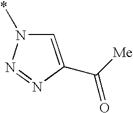 | Benzyl (3R)-3-[2-(4-acetyl-1H-1,2,3- triazol-1-yl)ethyl]piperazine-1- carboxylate hydrochloride | 358 |

TABLE 7-8

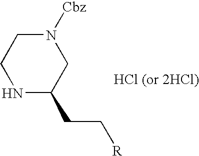

| Ref. Ex. No. | R | Compound | MS(ESI+) |
|---|---|---|---|
| 493 | 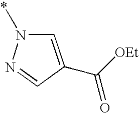 | Benzyl (3R)-3-{2-[4-(ethoxycarbonyl)- 1H-pyrazol-1-yl]ethyl}piperazine-1- carboxylate hydrochloride | 387 |
| 494 | 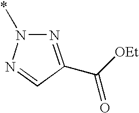 | Benzyl (3R)-3-{2-[4-(ethoxycarbonyl)- 2H-1,2,3-triazol-2-yl]ethyl}piperazine- 1-carboxylate hydrochloride | 388 |
| 495 | 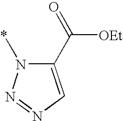 | Benzyl (3R)-3-{2-[5-(ethoxycarbonyl)- 1H-1,2,3-triazol-1-yl]ethyl}piperazine- 1-carboxylate hydrochloride | 388 |
| 496 | 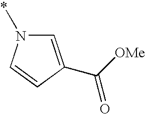 | Benzyl (3R)-3-{2-[3-(methoxy- carbonyl)-1H-pyrrol-1-yl]ethyl} piperazine-1-carboxylate hydrochloride | 372 |
| 497 | 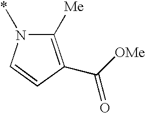 | Benzyl (3R)-3-{2-[3-(ethoxycarbonyl)- 2-methyl-1H-pyrrol-1-yl]ethyl} piperazine-1-carboxylate hydrochloride | 400 |
| 498 | 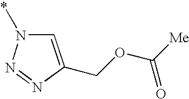 | Benzyl (3R)-3-(2-{4-[(acetyloxy) methyl]-1H-1,2,3-triazol-1-yl}ethyl) piperazine-1-carboxylate hydrochloride | 388 |
| 499 | 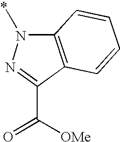 | Benzyl (3R)-3-{2-[3-(methoxy- carbonyl)-1H-indazol-1-yl]ethyl} piperazine-1-carboxylate hydrochloride | 423 |
| 500 | 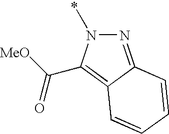 | Benzyl (3R)-3-{2-[3-(methoxy- carbonyl)-2H-indazol-2-yl]ethyl} piperazine-1-carboxylate hydrochloride | 423 |
| 501 | 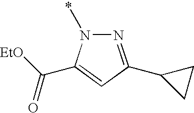 | Benzyl (3R)-3-{2-[3-cyclopropyl-5- (ethoxycarbonyl)-1H-pyrazol-1-yl] ethyl}piperazine-1-carboxylate hydrochloride | 427 |
| 502 | 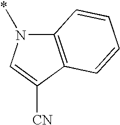 | Benzyl (3R)-3-[2-(3-cyano-1H-indol-1- yl)ethyl]piperazine-1-carboxylate hydrochloride | 389 |

### Reference Example 503

### 1-tert-Butyl 4-benzyl (2R)-2-(3-hydroxypropyl)piperazine-1,4-dicarboxylate

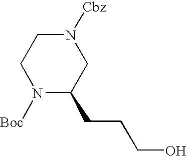

tert-Butyl (2R)-4-benzyl-2-(3-hydroxypropyl)piperazine-1-carboxylate (8.0 g) was dissolved in methanol (100 ml), 20% palladium hydroxide-carbon (50% containing water, 4.0 g) was added thereto, and the mixture was subjected to catalytic reduction at ambient temperature and normal pressure for 12 hr. The catalyst was filtered off, and the filtrate was concentrated under reduced pressure. The residue was dissolved in ethyl acetate (70 ml), and the solution was ice-cooled. Benzyl chloroformate (4.1 g), sodium carbonate (2.8 g) and water (35 ml) were added, and the mixture was stirred at 0° C. for 15 min, and then at room temperature for 1 hr. The reaction mixture was diluted with water, and the mixture was extracted with ethyl acetate. The extract was washed with saturated brine, and dried over anhydrous sodium sulfate, and the solvent was evaporated under reduced pressure. The residue was subjected to silica gel column chromatography, and the fraction eluted with ethyl acetate-methanol (1:1) was concentrated under reduced pressure to give the object compound (8.1 g) as an oil.

MS (ESI+, m/e) 379 (M+1)

### Reference Example 504

### 1-tert-Butyl 4-benzyl (2R)-2-{3-[(methylsulfonyl)oxy]propyl}piperazine-1,4-dicarboxylate

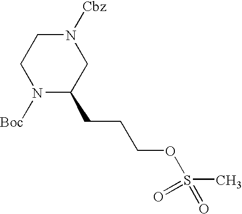

1-tert-Butyl 4-benzyl (2R)-2-(3-hydroxypropyl)piperazine-1,4-dicarboxylate (2.0 g) was dissolved in THF (10 ml), and the solution was ice-cooled. Triethylamine (1.1 ml) and methanesulfonyl chloride (510 μl) were added, and the mixture was stirred at room temperature for 2 hr. The reaction mixture was poured into saturated aqueous sodium hydrogen carbonate, and the mixture was extracted with ethyl acetate. The extract was washed with saturated brine, and dried over sodium sulfate, and the solvent was evaporated under reduced pressure. The residue was subjected to silica gel column chromatography, and the fraction eluted with ethyl acetate-hexane (1:1) was concentrated under reduced pressure to give the object compound (2.1 g) as an amorphous solid.

MS (ESI+, m/e) 457 (M+1)

### Reference Example 505

### Benzyl (3R)-3-(3-hydroxypropyl)piperazine-1-carboxylate

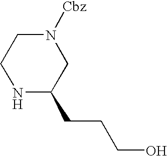

1-tert-Butyl 4-benzyl (2R)-2-(3-hydroxypropyl)piperazine-1,4-dicarboxylate (250 mg) was dissolved in chloroform (2 ml), TFA (2 ml) was added, and the mixture was stirred at room temperature for 1 hr. The solvent was evaporated under reduced pressure, and the residue was diluted with ethyl acetate. The mixture was washed with saturated aqueous sodium hydrogen carbonate, and dried over anhydrous sodium sulfate. The solvent was evaporated under reduced pressure to give the object compound (200 mg) as an oil.

MS (ESI+, m/e) 279 (M+1)

In the same manner as in Reference Example 255, the following compound (Reference Example 506) was obtained.

### Reference Example 506

### 1-tert-Butyl 4-benzyl (2R)-2-(3-phenoxypropyl)piperazine-1,4-dicarboxylate

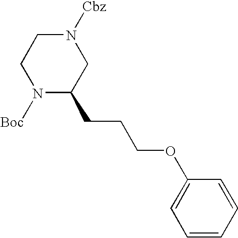

MS (ESI+, m/e) 455 (M+1)

In the same manner as in Reference Example 380, the following compound (Reference Example 507) was obtained.

### Reference Example 507

### 1-tert-Butyl 4-benzyl (2R)-2-[3-(1H-indazol-1-yl)propyl]piperazine-1,4-dicarboxylate and 1-tert-butyl 4-benzyl (2R)-2-[3-(2H-indazol-2-yl)propyl]piperazine-1,4-dicarboxylate

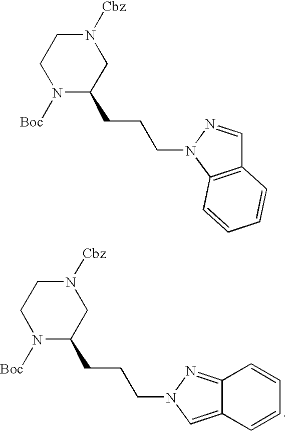

MS (ESI+, m/e) 479 (M+1)

MS (ESI+, m/e) 479 (M+1)

In the same manner as in Reference Example 383, the following compounds (Reference Examples 508-510) were obtained.

### Reference Example 508

### Benzyl (3R)-3-(3-phenoxypropyl)piperazine-1-carboxylate

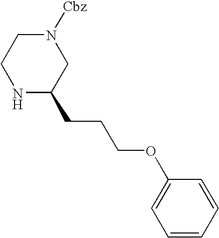

MS (ESI+, m/e) 355 (M+1)

### Reference Example 509

### Benzyl (3R)-3-[3-(1H-indazol-1-yl)propyl]piperazine-1-carboxylate

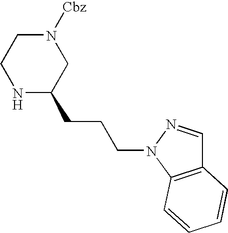

MS (ESI+, m/e) 379 (M+1)

### Reference Example 510

### Benzyl (3R)-3-[3-(2H-indazol-2-yl)propyl]piperazine-1-carboxylate

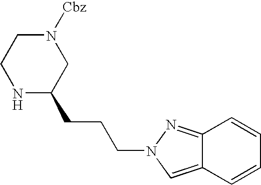

MS (ESI+, m/e) 379 (M+1)

### Reference Example 511

### Benzyl (3R)-3-{3-[5-(ethoxycarbonyl)-3-methyl-1H-pyrazol-1-yl]propyl}piperazine-1-carboxylate

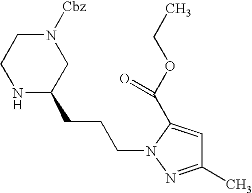

1-tert-Butyl 4-benzyl (2R)-2-(3-hydroxypropyl)piperazine-1,4-dicarboxylate (500 mg), ethyl 5-methyl-1H-pyrazole-3-carboxylate (550 mg) and tri-tert-butylphosphine (267 mg) were dissolved in toluene (20 ml), ADDP (420 mg) was added, and the mixture was stirred at room temperature for 12 hr. The reaction mixture was poured into saturated aqueous sodium hydrogen carbonate, and the mixture was extracted with ethyl acetate. The extract was washed with saturated brine, and dried over anhydrous sodium sulfate, and the solvent was evaporated under reduced pressure. The residue was subjected to silica gel column chromatography, and the fraction eluted with ethyl acetate-hexane (1:1) was concentrated under reduced pressure. The residue was dissolved in ethyl acetate (2 ml), 4N hydrogen chloride-ethyl acetate solution (2 ml) was added, and the mixture was stirred for 1 hr. The solvent was evaporated under reduced pressure, and the residue was dissolved in ethyl acetate. The solution was washed successively with saturated aqueous sodium hydrogen carbonate and saturated brine, and the solvent was evaporated under reduced pressure to give the object compound (140 mg) as an oil.

MS (ESI+, m/e) 415 (M+1)

### Reference Example 512

### tert-Butyl (3R)-3-benzyl-3-methylpiperazine-1-carboxylate

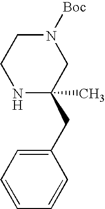

(2R)-2-Benzyl-2-methylpiperazine (1.10 g) and triethylamine (1.61 ml) was dissolved in THF (50 ml), and the solution was ice-cooled. A solution of di-tert-butyl bicarbonate (1.61 ml) in THF (10 ml) was added over 30 min, and the mixture was stirred at 0° C. for 3 hr. The solvent was evaporated under reduced pressure, to the residue was added saturated aqueous sodium hydrogen carbonate, and the mixture was extracted with ethyl acetate. The extract was washed with saturated brine, dried over anhydrous magnesium sulfate, and concentrated under reduced pressure. The residue was subjected to basic silica gel column chromatography, and the fraction eluted with ethyl acetate-hexane (4:1) was concentrated under reduced pressure to give the object compound (1.06 g).

MS (ESI+, m/e) 291 (M+1)

### Reference Example 513

### tert-Butyl (3R)-3-benzyl-4-({1-[(1S,2S)-2-(benzyloxy)cyclohexyl]-5-phenyl-1H-imidazol-4-yl}carbonyl)piperazine-1-carboxylate

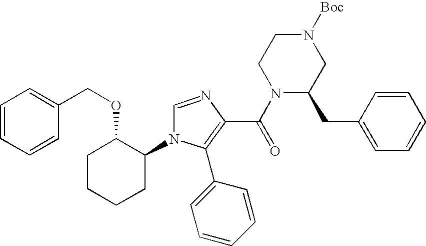

1-[(1S,2S)-2-(Benzyloxy)cyclohexyl]-5-phenyl-1H-imidazole-4-carboxylic acid (376 mg) was suspended in DMF (10 ml), tert-butyl (3R)-3-benzylpiperazine-1-carboxylate (332 mg), WSC.HCl (288 mg) and HOBt (184 mg) were added, and the mixture was stirred at room temperature for 12 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate was concentrated under reduced pressure to give the object compound (762 mg) as an amorphous solid.

MS (ESI+, m/e) 635 (M+1)

In the same manner as in Reference Example 513, the following compounds (Reference Examples 514-515) were obtained.

### Reference Example 514

### tert-Butyl (3R)-3-benzyl-4-({1-[(1R,2R)-2-(benzyloxy)cyclohexyl]-5-phenyl-1H-imidazol-4-yl}carbonyl)piperazine-1-carboxylate

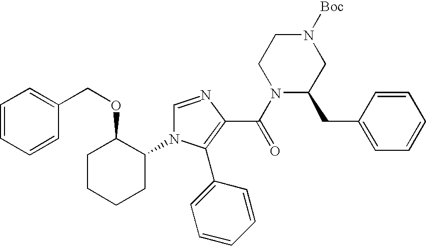

MS (ESI+, m/e) 635 (M+1)

### Reference Example 515

### tert-Butyl (3R)-4-({1-[(1S,2R)-2-azidocyclohexyl]-5-phenyl-1H-imidazol-4-yl}carbonyl)-3-benzylpiperazine-1-carboxylate

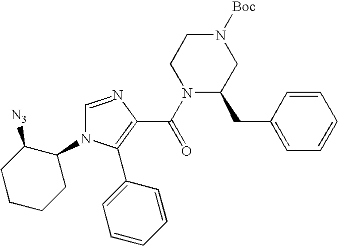

MS (ESI+, m/e) 570 (M+1)

### Reference Example 516

### tert-Butyl (3R)-3-benzyl-4-({5-(3-fluorophenyl)-1-[(1S,2S)-2-hydroxycyclohexyl]-1H-imidazol-4-yl}carbonyl)piperazine-1-carboxylate

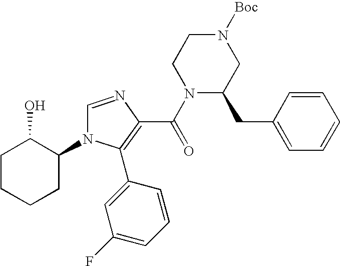

To a solution of 5-(3-fluorophenyl)-1-[(1S,2S)-2-hydroxycyclohexyl]-1H-imidazole-4-carboxylic acid (304 mg) in DMF (8 ml) were added tert-butyl (3R)-3-benzylpiperazine-1-carboxylate (332 mg), WSC.HCl (288 mg) and HOBt (184 mg), and the mixture was stirred at 60° C. for 3 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate-methanol (19:1) was concentrated under reduced pressure to give the object compound (380 mg) as an amorphous solid.

MS (ESI+, m/e) 563 (M+1)

### Reference Example 517

### tert-Butyl (3R)-3-benzyl-4-({1-[(1R,2S)-2-hydroxy-2-(methoxymethyl)cyclohexyl]-5-phenyl-1H-imidazol-4-yl}carbonyl)piperazine-1-carboxylate

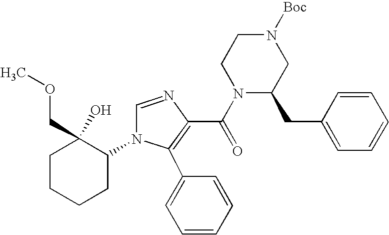

A solution of 1-[(1R,2S)-2-hydroxy-2-(methoxymethyl)cyclohexyl]-5-phenyl-1H-imidazole-4-carboxylic acid (3.30 g), tert-butyl (3R)-3-benzylpiperazine-1-carboxylate (3.32 g), WSC.HCl (2.88 g) and HOBt (2.30 g) in DMF (100 ml) was stirred at 60° C. for 5 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to basic silica gel column chromatography, and the fraction eluted with ethyl acetate was concentrated under reduced pressure to give the object compound (5.45 g) as an amorphous solid.

MS (ESI+, m/e) 589 (M+1)

### Reference Example 518

### (1S,2R)-2-(4-{[(2R)-4-Benzyl-2-(2-hydroxyethyl)piperazin-1-yl]carbonyl}-5-phenyl-1H-imidazol-1-yl)-1-(methoxymethyl)cyclohexanol

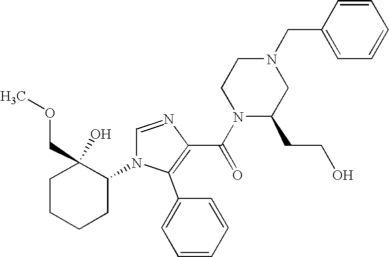

1-[(1R,2S)-2-Hydroxy-2-(methoxymethyl)cyclohexyl]-5-phenyl-1H-imidazole-4-carboxylic acid (330 mg) was suspended in DMF (5 ml), 2-[(2R)-4-benzylpiperazin-2-yl]ethanol (264 mg), WSC.HCl (230 mg) and HOBt (168 mg) were added, and the mixture was stirred at room temperature for 12 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate was concentrated under reduced pressure to give the object compound (355 mg) as an amorphous solid.

MS (ESI+, m/e) 533 (M+1)

### Reference Example 519

### Benzyl (3R)-3-[2-(2-fluorophenoxy)ethyl]-4-({1-[(1R,2S)-2-hydroxy-2-(methoxymethyl)cyclohexyl]-5-phenyl-1H-imidazol-4-yl}carbonyl)piperazine-1-carboxylate

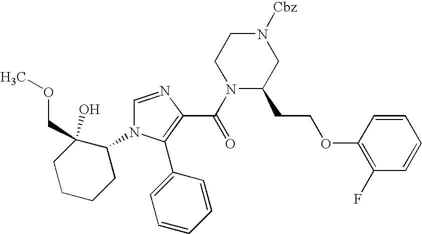

1-tert-Butyl 4-benzyl (2R)-2-[2-(2-fluorophenoxy)ethyl]piperazine-1,4-dicarboxylate (210 mg) was dissolved in ethyl acetate (1 ml), 4N hydrogen chloride-ethyl acetate solution (1 ml) was added, and the mixture was stirred at room temperature for 1 hr. The reaction mixture was concentrated under reduced pressure, and the residue was suspended in toluene (1 ml). The suspension was concentrated again, and the residue was vacuum-dried. This was suspended in DMF (2 ml), 1-[(1R,2S)-2-hydroxy-2-(methoxymethyl)cyclohexyl]-5-phenyl-1H-imidazole-4-carboxylic acid (135 mg), WSC.HCl (118 mg), HOBt (94 mg) and triethylamine (83 mg) were added, and the mixture was stirred at room temperature for 12 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate-hexane (1:1-1:0) was concentrated under reduced pressure to give the object compound (205 mg) as an amorphous solid.

MS (ESI+, m/e) 671 (M+1)

### Reference Example 520

### Benzyl (3R)-4-({1-[(1R,2S)-2-hydroxy-2-(methoxymethyl)cyclohexyl]-5-phenyl-1H-imidazol-4-yl}carbonyl)-3-{2-[4-(methoxycarbonyl)phenoxy]ethyl}piperazine-1-carboxylate

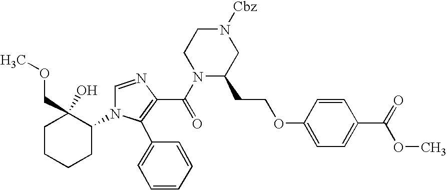

1-tert-Butyl 4-benzyl (2R)-2-{2-[4-(methoxycarbonyl)phenoxy]ethyl}piperazine-1,4-dicarboxylate (409 mg) was dissolved in methanol (2 ml), 4N hydrogen chloride-ethyl acetate solution was added, and the mixture was stirred at room temperature for 5 hr, and concentrated under reduced pressure. The residue was suspended in DMF (5 ml), 1-[(1R,2S)-2-hydroxy-2-(methoxymethyl)cyclohexyl]-5-phenyl-1H-imidazole-4-carboxylic acid (271 mg), WSC.HCl (236 mg), HOBt (151 mg) and triethylamine (229 μl) were added, and the mixture was stirred at 60° C. for 5 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate was concentrated under reduced pressure to give the object compound (486 mg) as an amorphous solid.

MS (ESI+, m/e) 711 (M+1)

In the same manner as in Reference Example 520, the following compounds (Reference Examples 521-525) were obtained.

### Reference Example 521

### Benzyl (3R)-4-({1-[(1R,2S)-2-hydroxy-2-(methoxymethyl)cyclohexyl]-5-phenyl-1H-imidazol-4-yl}carbonyl)-3-{2-[3-(methoxycarbonyl)phenoxy]ethyl}piperazine-1-carboxylate

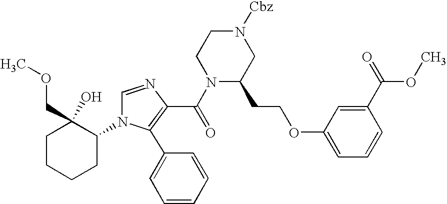

MS (ESI+, m/e) 711 (M+1)

### Reference Example 522

### Benzyl (3R)-4-({1-[(1R,2S)-2-hydroxy-2-(methoxymethyl)cyclohexyl]-5-phenyl-1H-imidazol-4-yl}carbonyl)-3-{2-[2-(methoxycarbonyl)phenoxy]ethyl}piperazine-1-carboxylate

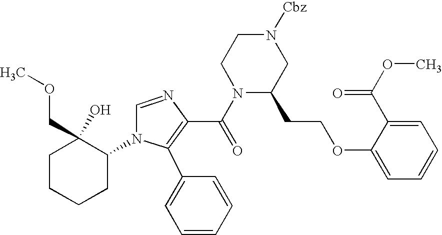

MS (ESI+, m/e) 711 (M+1)

### Reference Example 523

### Benzyl (3R)-4-({1-[(1R,2S)-2-hydroxy-2-(methoxymethyl)cyclohexyl]-5-phenyl-1H-imidazol-4-yl}carbonyl)-3-{2-[(1-oxidopyridin-3-yl)oxy]ethyl}piperazine-1-carboxylate

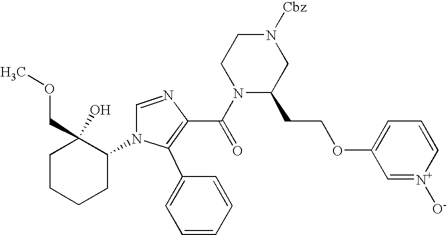

MS (ESI+, m/e) 669 (M+1)

### Reference Example 524

### Benzyl (3R)-3-{2-[4-(4-acetylpiperazin-1-yl)phenoxy]ethyl}-4-({1-[(1R,2S)-2-hydroxy-2-(methoxymethyl)cyclohexyl]-5-phenyl-1H-imidazol-4-yl}carbonyl)piperazine-1-carboxylate

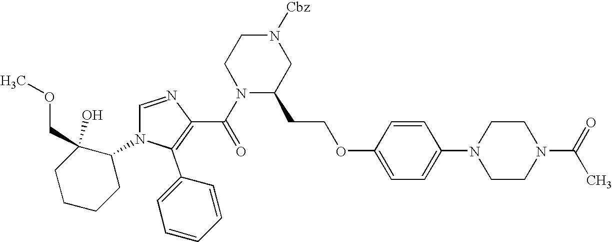

MS (ESI+, m/e) 779 (M+1)

### Reference Example 525

### Benzyl (3R)-3-[2-(4-cyano-2-methoxyphenoxy)ethyl]-4-({1-[(1R,2S)-2-hydroxy-2-(methoxymethyl)cyclohexyl]-5-phenyl-1H-imidazol-4-yl}carbonyl)piperazine-1-carboxylate

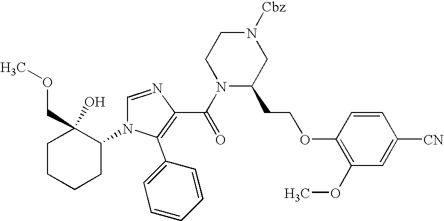

MS (ESI+, m/e) 708 (M+1)

### Reference Example 526

### Benzyl (3R)-4-({1-[(1R,2S)-2-hydroxy-2-(methoxymethyl)cyclohexyl]-5-phenyl-1H-imidazol-4-yl}carbonyl)-3-[2-(1H-indazol-1-yl)ethyl]piperazine-1-carboxylate

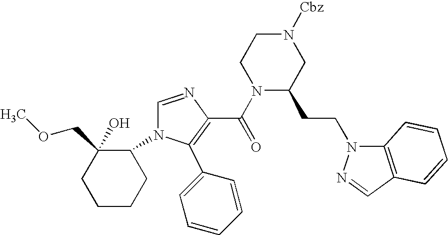

1-[(1R,2S)-2-Hydroxy-2-(methoxymethyl)cyclohexyl]-5-phenyl-1H-imidazole-4-carboxylic acid (110 mg) was suspended in DMF (5 ml), benzyl (3R)-3-[2-(1H-indazol-1-yl)ethyl]piperazine-1-carboxylate (121 mg), WSC.HCl (126 mg) and HOBt (202 mg) were added, and the mixture was stirred at 60° C. for 10 hr. The reaction mixture was poured into saturated aqueous sodium hydrogen carbonate, and the mixture was extracted with ethyl acetate. The extract was washed successively with 10% aqueous citric acid solution and saturated brine, and dried over anhydrous sodium sulfate, and the solvent was evaporated under reduced pressure. The residue was subjected to silica gel column chromatography, and the fraction eluted with ethyl acetate-methanol (9:1) was concentrated under reduced pressure to give the object compound (140 mg) as an amorphous solid.

MS (ESI+, m/e) 677 (M+1)

In the same manner as in Reference Example 526, the following compound (Reference Example 527) was obtained.

### Reference Example 527

### Benzyl (3R)-4-({1-[(1R,2S)-2-hydroxy-2-(methoxymethyl)cyclohexyl]-5-phenyl-1H-imidazol-4-yl}carbonyl)-3-[2-(2H-indazol-2-yl)ethyl]piperazine-1-carboxylate

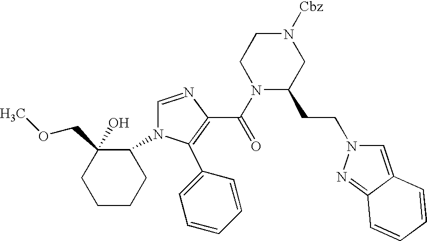

MS (ESI+, m/e) 677 (M+1)

### Reference Example 528

### (1R,2S)-2-[4-({(2R)-4-Benzyl-2-[(E)-2-cyclopropylvinyl]piperazin-1-yl}carbonyl)-5-phenyl-1H-imidazol-1-yl]cyclohexanol

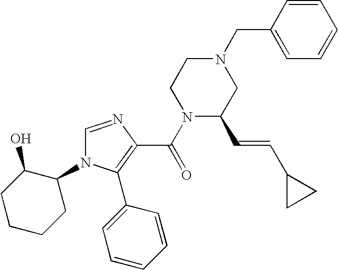

A solution of 1-[(1S,2R)-2-hydroxycyclohexyl]-5-phenyl-1H-imidazole-4-carboxylic acid (144 mg), (3R)-1-benzyl-3-[(E)-2-cyclopropylvinyl]piperazine (125 mg), WSC.HCl (125 mg), HOBt (23 mg), N,N-diisopropylethylamine (181 μl) and DMAP (12 mg) in DMF (2 ml) was stirred at room temperature for 12 hr, and poured into saturated aqueous sodium hydrogen carbonate, and the mixture was extracted with ethyl acetate. The extract was washed with saturated brine, and dried over anhydrous sodium sulfate, and the solvent was evaporated under reduced pressure. The residue was subjected to silica gel column chromatography, and the fraction eluted with ethyl acetate-hexane-methanol (1:1:0-4:0:1) was concentrated under reduced pressure to give the object compound (110 mg) as an amorphous solid.

MS (ESI+, m/e) 511 (M+1)

### Reference Example 529

### tert-Butyl (3R)-3-benzyl-4-[(1-cyclohexyl-5-phenyl-1H-imidazol-4-yl)carbonyl]piperazine-1-carboxylate

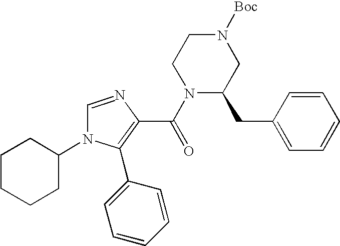

Methyl 1-cyclohexyl-5-phenyl-1H-imidazole-4-carboxylate (240 mg) was dissolved in ethanol-THF (1:1, 10 ml), lithium hydroxide monohydrate (30 mg) was added, and the mixture was stirred at 80° C. for 2 hr. The reaction mixture was concentrated under reduced pressure, the residue was suspended in ethanol, and the suspension was again concentrated under reduced pressure. This was suspended in DMF (15 ml), tert-butyl (3R)-3-benzylpiperazine-1-carboxylate (240 mg), WSC.HCl (178 mg) and HOBt (142 mg) were added, and the mixture was stirred at room temperature for 12 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate was concentrated under reduced pressure to give the object compound (321 mg) as an amorphous solid.

MS (ESI+, m/e) 529 (M+1)

In the same manner as in Reference Example 529, the following compounds (Reference Examples 530-536) were obtained.

### Reference Example 530

### tert-Butyl (3R)-3-benzyl-4-{[1-(trans-2-hydroxycyclopentyl)-5-phenyl-1H-imidazol-4-yl]carbonyl}piperazine-1-carboxylate

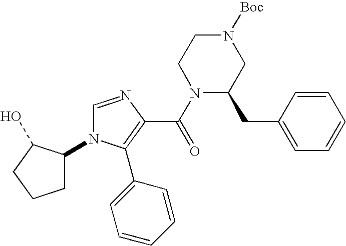

MS (ESI+, m/e) 531 (M+1)

### Reference Example 531

### tert-Butyl (3R)-3-benzyl-4-{[1-(cis-2-hydroxycyclohexyl)-5-phenyl-1H-imidazol-4-yl]carbonyl}piperazine-1-carboxylate

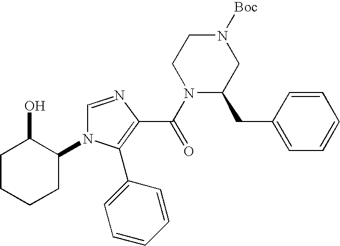

MS (ESI+, m/e) 545 (M+1)

### Reference Example 532

### {(2S)-4-Benzyl-1-[(1-cyclopentyl-5-phenyl-1H-imidazol-4-yl)carbonyl]piperazin-2-yl}(cyclopropyl)methanol

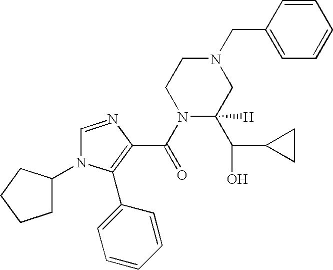

MS (ESI+, m/e) 485 (M+1)

### Reference Example 533

### trans-2-(4-{[(2R)-4-Benzyl-2-isobutylpiperazin-1-yl]carbonyl}-5-phenyl-1H-imidazol-1-yl)cyclopentanol

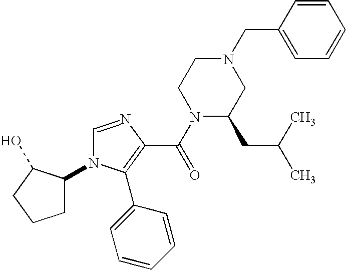

MS (ESI+, m/e) 487 (M+1)

### Reference Example 534

### N-{[(2R)-4-Benzyl-1-({1-[2-(ethoxymethyl)-2-hydroxycyclohexyl]-5-phenyl-1H-imidazol-4-yl}carbonyl)piperazin-2-yl]methyl}benzamide

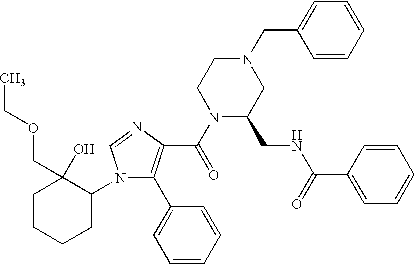

MS (ESI+, m/e) 636 (M+1)

### Reference Example 535

### 2-[4-({(2S)-4-Benzyl-2-[(benzyloxy)methyl]piperazin-1-yl}carbonyl)-5-phenyl-1H-imidazol-1-yl]-1-(methoxymethyl)cyclohexanol

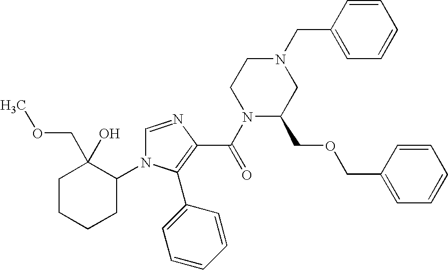

MS (ESI+, m/e) 609 (M+1)

### Reference Example 536

### tert-Butyl (3R)-3-benzyl-4-{[1-(trans-2-hydroxycyclohexyl)-2-methyl-5-phenyl-1H-imidazol-4-yl]carbonyl}piperazine-1-carboxylate

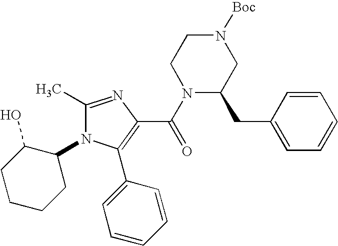

MS (ESI+, m/e) 559 (M+1)

### Reference Example 537

### tert-Butyl (3R)-3-benzyl-4-{[1-(trans-2-hydroxycycloheptyl)-5-phenyl-1H-imidazol-4-yl]carbonyl}piperazine-1-carboxylate

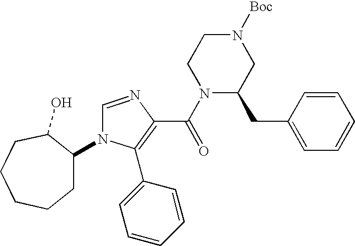

A mixture of ethyl 1-(trans-2-hydroxycycloheptyl)-5-phenyl-1H-imidazole-4-carboxylate (860 mg), lithium hydroxide monohydrate (165 mg), ethanol (10 ml) and water (6 ml) was stirred at 65° C. for 3 hr, and concentrated under reduced pressure. The residue was mixed with tert-butyl (3R)-3-benzylpiperazine-1-carboxylate (868 mg), WSC.HCl (1.00 g), HOBt (1.60 g) and DMF (15 ml), and the mixture was stirred at 50° C. for 12 hr, and poured into water. The obtained crystals were collected by filtration, and washed successively with water and ethyl acetate to give the object compound (421 mg). The filtrate was extracted with ethyl acetate. The extract was washed with saturated brine, and dried over anhydrous magnesium sulfate, and the solvent was evaporated under reduced pressure. The residue was subjected to silica gel column chromatography, and the fraction eluted with ethyl acetate-methanol (1:0-9:1) was concentrated under reduced pressure to give the object compound (754 mg). The yield of the obtained object compound was 1.17 g in total.

MS (ESI+, m/e) 559 (M+1)

### Reference Example 538

### (1S,2S)-2-(4-{[(2R)-2,4-Dibenzylpiperazin-1-yl]carbonyl}-5-phenyl-1H-imidazol-1-yl)cyclohexanamine

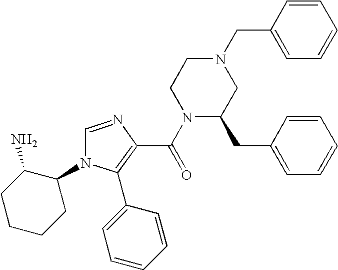

Ethyl 1-{(1S,2S)-2-[(tert-butoxycarbonyl)amino]cyclohexyl}-5-phenyl-1H-imidazole-4-carboxylate (400 mg) was dissolved in methanol-water (2:1, 6 ml), lithium hydroxide monohydrate (63 mg) was added, and the mixture was stirred at 65° C. for 3 hr. The reaction mixture was concentrated under reduced pressure, and the residue was suspended in ethanol. The suspension was concentrated again, and the residue was vacuum-dried. This was suspended in DMF (8 ml), (3R)-1,3-dibenzylpiperazine (320 mg), WSC.HCl (383 mg) and HOBt (613 g) were added, and the mixture was stirred at room temperature for 12 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate-hexane (3:7-7:3) was concentrated under reduced pressure to give tert-butyl [(1S,2S)-2-(4-{[(2R)-2,4-dibenzylpiperazin-1-yl]carbonyl}-5-phenyl-1H-imidazol-1-yl)cyclohexyl]carbamate (550 mg) as an amorphous solid. 539 mg therefrom was dissolved in dichloromethane (2 ml), TFA (1 ml) was added, and the mixture was stirred at room temperature for 1 hr. The reaction mixture was concentrated under reduced pressure, and the residue was neutralized with saturated aqueous sodium hydrogen carbonate. The liberated oil was extracted with ethyl acetate, and dried over anhydrous sodium sulfate. The solvent was evaporated under reduced pressure to give the object compound (450 mg) as an amorphous solid.

MS (ESI+, m/e) 534 (M+1)

### Reference Example 539

### Ethyl [(1S,2S)-2-(4-{[(2R)-4-benzyl-2-(3,5-difluorobenzyl)piperazin-1-yl]carbonyl}-5-phenyl-1H-imidazol-1-yl)cyclohexyl]carbamate

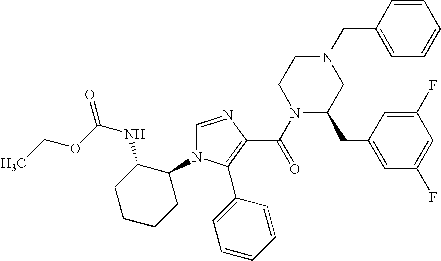

Ethyl 1-{(1S,2S)-2-[(ethoxycarbonyl)amino]cyclohexyl}-5-phenyl-1H-imidazole-4-carboxylate (424 mg) was dissolved in ethanol-water (2:1, 6 ml), lithium hydroxide monohydrate (69 mg) was added, and the mixture was stirred at 65° C. for 3 hr. The reaction mixture was concentrated under reduced pressure, and the residue was suspended in ethanol. The suspension was concentrated again, and the residue was vacuum-dried. This was suspended in DMF (5 ml), (3R)-1-benzyl-3-(3,5-difluorobenzyl)piperazine (333 mg), WSC.HCl (422 mg) and HOBt (674 mg) were added, and the mixture was stirred at room temperature for 12 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate-hexane (3:7-7:3) was concentrated under reduced pressure to give the object compound (530 mg) as an amorphous solid.

MS (ESI+, m/e) 642 (M+1)

### Reference Example 540

### tert-Butyl (3R)-3-benzyl-4-{[1-(2-oxo-1-oxa-3-azaspiro[4.5]deca-6-yl)-5-phenyl-1H-imidazol-4-yl]carbonyl}piperazine-1-carboxylate

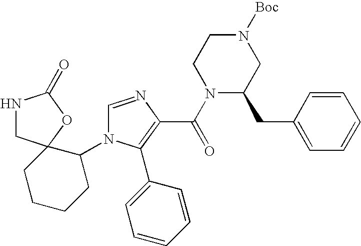

Ethyl 1-(2-{[(ethoxycarbonyl)amino]methyl}-2-hydroxycyclohexyl)-5-phenyl-1H-imidazole-4-carboxylate (300 mg) was dissolved in ethanol-water (2:1, 6 ml), lithium hydroxide monohydrate (45 mg) was added, and the mixture was stirred at 65° C. for 3 hr. The reaction mixture was concentrated under reduced pressure, and the residue was suspended in ethanol. The suspension was concentrated again, and the residue was vacuum-dried. This was suspended in DMF (8 ml), tert-butyl (3R)-3-benzylpiperazine-1-carboxylate (240 mg), WSC.HCl (277 mg) and HOBt (442 mg) were added, and the mixture was stirred at room temperature for 12 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate-hexane (3:7-7:3) was concentrated under reduced pressure to give the object compound (300 mg) as an amorphous solid.

MS (ESI+, m/e) 600 (M+1)

### Reference Example 541

### tert-Butyl (3R)-3-benzyl-4-[(1-{(1S,2S)-2-[(ethoxycarbonyl)amino]cyclohexyl}-5-phenyl-1H-imidazol-4-yl)carbonyl]piperazine-1-carboxylate

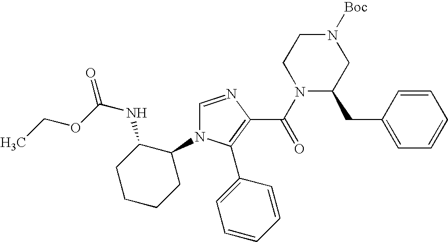

Ethyl 1-{(1S,2S)-2-[(ethoxycarbonyl)amino]cyclohexyl}-5-phenyl-1H-imidazole-4-carboxylate (540 mg) was dissolved in ethanol-water (2:1, 9 ml), lithium hydroxide monohydrate (88 mg) was added, and the mixture was stirred at 65° C. for 3 hr. The reaction mixture was concentrated under reduced pressure, and the residue was suspended in ethanol. The suspension was concentrated again, and the residue was vacuum-dried. This was suspended in DMF (10 ml), tert-butyl (3R)-3-benzylpiperazine-1-carboxylate (464 mg), WSC.HCl (537 mg) and HOBt (858 mg) were added, and the mixture was stirred at room temperature for 12 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate-hexane (3:7-7:3) was concentrated under reduced pressure to give the object compound (385 mg) as an amorphous solid.

MS (ESI+, m/e) 616 (M+1)

### Reference Example 542

### tert-Butyl (3R)-3-benzyl-4-({1-[(1S,2R)-2-(chloromethyl)-2-hydroxycyclohexyl]-5-phenyl-1H-imidazol-4-yl}carbonyl)piperazine-1-carboxylate

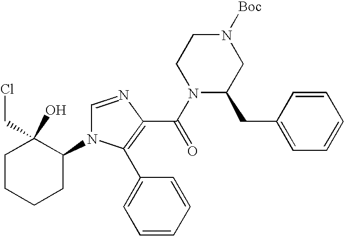

Ethyl 1-[(3R,4S)-1-oxaspiro[2.5]oct-4-yl]-5-phenyl-1H-imidazole-4-carboxylate (1.31 g) was dissolved in methanol-THF (1:4, 25 ml), lithium hydroxide monohydrate (505 mg) and water (10 ml) were added, and the mixture was stirred at 50° C. for 15 hr. The mixture was neutralized with 1N hydrochloric acid, and extracted with ethyl acetate. The extract was washed with saturated brine, dried over anhydrous magnesium sulfate, and concentrated under reduced pressure. The residue was suspended in DMF (10 ml), tert-butyl 3-benzylpiperazine-1-carboxylate (817 mg), WSC.HCl (1.13 g) and HOBt (1.36 g) were added, and the mixture was stirred at 60° C. for 15 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate was concentrated under reduced pressure to give the object compound (914 mg) as an amorphous solid.

MS (ESI+, m/e) 593 (M+1)

### Reference Example 543

### tert-Butyl (3R)-3-benzyl-4-({1-[(1S,2S)-2-hydroxycyclohexyl]-5-phenyl-1H-imidazol-4-yl}carbonyl)piperazine-1-carboxylate

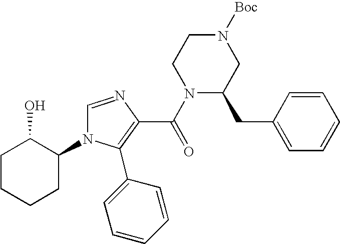

tert-Butyl (3R)-3-benzyl-4-({1-[(1S,2S)-2-(benzyloxy)cyclohexyl]-5-phenyl-1H-imidazol-4-yl}carbonyl)piperazine-1-carboxylate (500 mg) was dissolved in methanol (10 ml), 20% palladium hydroxide-carbon (50% containing water, 100 mg) was added thereto, and the mixture was subjected to catalytic reduction at ambient temperature and normal pressure for 12 hr. The catalyst was filtered off, and the filtrate was concentrated under reduced pressure. The residue was subjected to silica gel column chromatography, and the fraction eluted with ethyl acetate-methanol (4:1) was concentrated under reduced pressure to give the object compound (264 mg) as an amorphous solid.

MS (ESI+, m/e) 545 (M+1)

In the same manner as in Reference Example 543, the following compound (Reference Example 544) was obtained.

### Reference Example 544

### tert-Butyl (3R)-3-benzyl-4-({1-[(1R,2R)-2-hydroxycyclohexyl]-5-phenyl-1H-imidazol-4-yl}carbonyl)piperazine-1-carboxylate

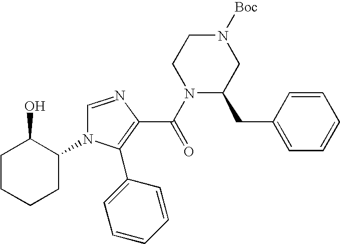

MS (ESI+, m/e) 545 (M+1)

### Reference Example 545

### tert-Butyl (3R)-4-({1-[(1S,2S)-2-(acetyloxy)cyclohexyl]-5-phenyl-1H-imidazol-4-yl}carbonyl)-3-benzylpiperazine-1-carboxylate

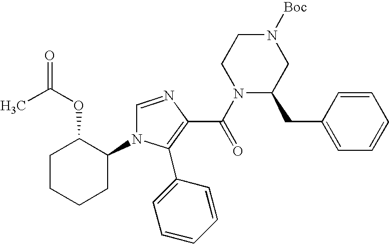

tert-Butyl (3R)-3-benzyl-4-({1-[(1S,2S)-2-hydroxycyclohexyl]-5-phenyl-1H-imidazol-4-yl}carbonyl)piperazine-1-carboxylate (272 mg) was dissolved in THF (10 ml), acetic acid (20 μl), WSC.HCl (144 mg) and DMAP (6 mg) were added, and the mixture was stirred at room temperature for 15 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate-methanol (19:1) was concentrated under reduced pressure to give the object compound (268 mg) as an amorphous solid.

MS (ESI+, m/e) 587 (M+1)

### Reference Example 546

### tert-Butyl (3R)-3-benzyl-4-[(1-{(1S,2R)-2-[(4-nitrobenzoyl)oxy]cyclohexyl}-5-phenyl-1H-imidazol-4-yl)carbonyl]piperazine-1-carboxylate

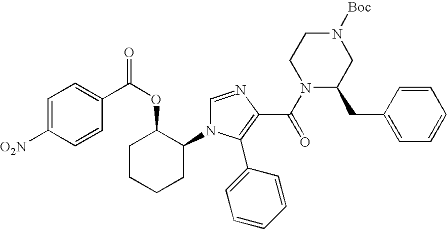

tert-Butyl (3R)-3-benzyl-4-({1-[(1S,2S)-2-hydroxycyclohexyl]-5-phenyl-1H-imidazol-4-yl}carbonyl)piperazine-1-carboxylate (250 mg) and 4-nitrobenzoic acid were dissolved in THF (20 ml), DTBAD (424 mg) and PS-triphenylphosphine resin (manufactured by Argonaut Technologies, 2.15 mmol/g, 856 mg) were added, and the mixture was stirred at room temperature for 15 hr. The insoluble material was filtered off, and the filtrate was concentrated under reduced pressure. The residue was subjected to silica gel column chromatography, and the fraction eluted with ethyl acetate was concentrated under reduced pressure to give the object compound (207 mg) as an amorphous solid.

MS (ESI+, m/e) 694 (M+1)

### Reference Example 547

### tert-Butyl (3R)-3-benzyl-4-({1-[(1S,2R)-2-hydroxycyclohexyl]-5-phenyl-1H-imidazol-4-yl}carbonyl)piperazine-1-carboxylate

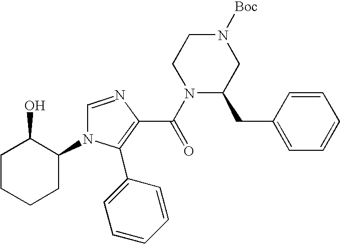

tert-Butyl (3R)-3-benzyl-4-[(1-{(1S,2R)-2-[(4-nitrobenzoyl)oxy]cyclohexyl}-5-phenyl-1H-imidazol-4-yl)carbonyl]piperazine-1-carboxylate (205 mg) was dissolved in methanol-THF (1:1, 10 ml), 8N aqueous sodium hydroxide solution (3 ml) was added, and the mixture was stirred at room temperature for 4 hr. The reaction mixture was concentrated under reduced pressure, and the residue was partitioned between ethyl acetate and water. The organic layer was washed with saturated brine, and dried over anhydrous sodium sulfate, and the solvent was evaporated under reduced pressure. The residue was subjected to silica gel column chromatography, and the fraction eluted with ethyl acetate-methanol (19:1) was concentrated under reduced pressure to give the object compound (117 mg) as an amorphous solid.

MS (ESI+, m/e) 545 (M+1)

### Reference Example 548

### tert-Butyl (3R)-3-benzyl-4-({1-[(1S,2S)-2-(3-methoxypropoxy)cyclohexyl]-5-phenyl-1H-imidazol-4-yl}carbonyl)piperazine-1-carboxylate

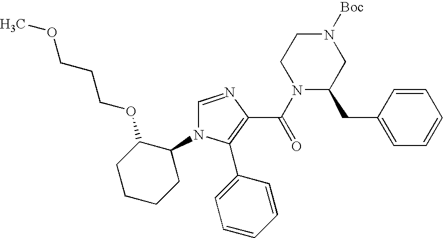

tert-Butyl (3R)-3-benzyl-4-({1-[(1S,2S)-2-hydroxycyclohexyl]-5-phenyl-1H-imidazol-4-yl}carbonyl)piperazine-1-carboxylate (163 mg) was dissolved in THF (2 ml), sodium hydride (60% in oil, 60 mg) was added thereto, and the mixture was stirred at room temperature for 30 min. After stirring, 1-bromo-3-methoxypropane (115 mg) was added thereto. The reaction mixture was heated under reflux for 15 hr, and concentrated under reduced pressure. Ethyl acetate was added to the residue, and the mixture was washed with saturated brine, dried over anhydrous magnesium sulfate, and concentrated under reduced pressure. The residue was subjected to silica gel column chromatography, and the fraction eluted with ethyl acetate-hexane (3:7-7:3) was concentrated under reduced pressure to give the object compound (66 mg) as an amorphous solid.

MS (ESI+, m/e) 617 (M+1)

In the same manner as in Reference Example 548, the following compounds (Reference Examples 549-552) were obtained.

### Reference Example 549

### tert-Butyl (3R)-4-({1-[(1S,2S)-2-(allyloxy)cyclohexyl]-5-phenyl-1H-imidazol-4-yl}carbonyl)-3-benzylpiperazine-1-carboxylate

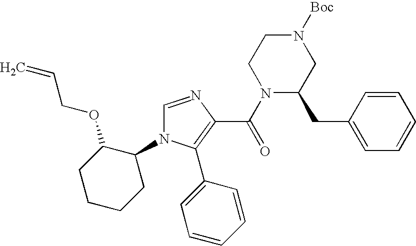

MS (ESI+, m/e) 585 (M+1)

### Reference Example 550

### tert-Butyl (3R)-3-benzyl-4-({5-phenyl-1-[(1S,2S)-2-propoxycyclohexyl]-1H-imidazol-4-yl}carbonyl)piperazine-1-carboxylate

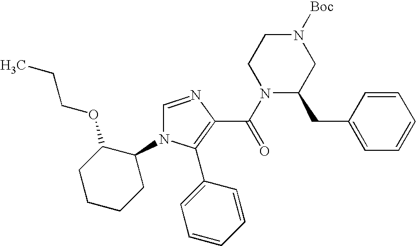

MS (ESI+, m/e) 587 (M+1)

### Reference Example 551

### tert-Butyl (3R)-3-benzyl-4-({1-[(1S,2S)-2-(2-methoxyethoxy)cyclohexyl]-5-phenyl-1H-imidazol-4-yl}carbonyl)piperazine-1-carboxylate

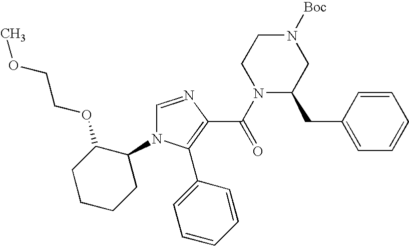

MS (ESI+, m/e) 603 (M+1)

### Reference Example 552

### tert-Butyl (3R)-3-benzyl-4-({1-[(1S,2S)-2-(4-methoxybutoxy)cyclohexyl]-5-phenyl-1H-imidazol-4-yl}carbonyl)piperazine-1-carboxylate

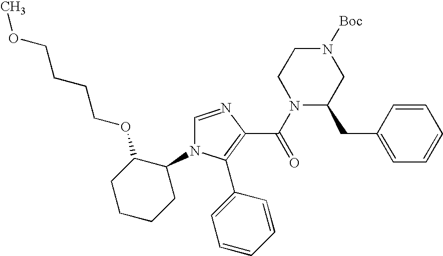

MS (ESI+, m/e) 631 (M+1)

### Reference Example 553

### tert-Butyl (3R)-4-[(1-{(1S,2S)-2-[3-(acetylamino)propoxy]cyclohexyl}-5-phenyl-1H-imidazol-4-yl)carbonyl]-3-benzylpiperazine-1-carboxylate

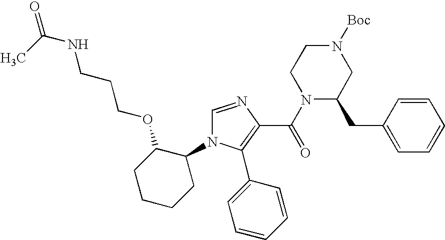

tert-Butyl (3R)-3-benzyl-4-({1-[(1S,2S)-2-hydroxycyclohexyl]-5-phenyl-1H-imidazol-4-yl}carbonyl)piperazine-1-carboxylate (191 mg) was dissolved in DMF (2 ml), sodium hydride (60% in oil, 70 mg) was added, and the mixture was stirred at room temperature for 30 min. After stirring, 1-(3-bromopropyl)-2,2,5,5-tetramethyl-1,2,5-azadisilolidine (245 mg) was added thereto. The mixture was stirred at 80° C. for 15 hr, and concentrated under reduced pressure. Ethyl acetate was added to the residue, and the mixture was washed with saturated brine, dried over anhydrous magnesium sulfate, and concentrated under reduced pressure. The residue was subjected to basic silica gel column chromatography, and the fraction eluted with ethyl acetate-methanol (1:0-9:1) was concentrated under reduced pressure to give tert-butyl (3R)-4-({1-[(1S,2S)-2-(3-aminopropoxy)cyclohexyl]-5-phenyl-1H-imidazol-4-yl}carbonyl)-3-benzylpiperazine-1-carboxylate (160 mg) as an amorphous solid. This was mixed with triethylamine (40 mg) and dichloromethane (2 ml), and the mixture was ice-cooled. Acetyl chloride (25 mg) was added thereto, and the mixture was stirred at 0° C. for 30 min. After stirring, the mixture was concentrated under reduced pressure. Ethyl acetate was added to the residue, and the mixture was washed successively with water and saturated brine, dried over anhydrous magnesium sulfate, and concentrated under reduced pressure. The residue was subjected to basic silica gel column chromatography, and the fraction eluted with ethyl acetate-hexane (1:1-1:0) was concentrated under reduced pressure to give the object compound (120 mg) as an amorphous solid.

MS (ESI+, m/e) 644 (M+1)

### Reference Example 554

### tert-Butyl (3R)-3-benzyl-4-[(1-{cis-2-[(4-nitrobenzoyl)oxy]cycloheptyl}-5-phenyl-1H-imidazol-4-yl)carbonyl]piperazine-1-carboxylate

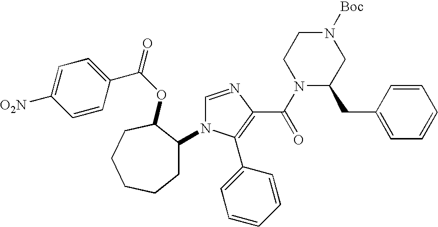

A mixture of tert-butyl (3R)-3-benzyl-4-{[1-(trans-2-hydroxycycloheptyl)-5-phenyl-1H-imidazol-4-yl]carbonyl}piperazine-1-carboxylate (280 mg), 4-nitrobenzoic acid (335 mg), PS-triphenylphosphine resin (manufactured by Argonaut Technologies, 1.99 mmol/g) (930 mg), DTBAD (460 mg) and THF (20 ml) was stirred at room temperature for 3 days, and the insoluble material was filtered off. The filtrate was diluted with ethyl acetate, and washed successively with 0.5N aqueous sodium hydroxide solution and saturated brine. The organic layer was dried over anhydrous magnesium sulfate, and the solvent was evaporated under reduced pressure. The residue was subjected to silica gel column chromatography, and the fraction eluted with ethyl acetate-hexane (1:4-1:0) was concentrated under reduced pressure to give the object compound (224 mg).

MS (ESI+, m/e) 708 (M+1)

### Reference Example 555

### tert-Butyl (3R)-3-benzyl-4-({1-[cis-2-hydroxycycloheptyl]-5-phenyl-1H-imidazol-4-yl}carbonyl)piperazine-1-carboxylate

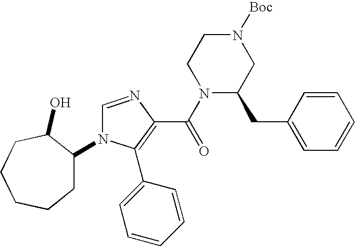

A mixture of tert-butyl (3R)-3-benzyl-4-[(1-{cis-2-[(4-nitrobenzoyl)oxy]cycloheptyl}-5-phenyl-1H-imidazol-4-yl)carbonyl]piperazine-1-carboxylate (220 mg), 1N aqueous sodium hydroxide solution (1.5 ml) and ethanol (6 ml) was stirred at room temperature for 13 hr, and poured into water, and the mixture was extracted with ethyl acetate. The extract was washed with saturated brine, and dried over anhydrous magnesium sulfate, and the solvent was evaporated under reduced pressure. The residue was subjected to basic silica gel column chromatography, and the fraction eluted with ethyl acetate-methanol (1:0-9:1) was concentrated under reduced pressure to give the object compound (171 mg).

MS (ESI+, m/e) 559 (M+1)

### Reference Example 556

### tert-Butyl (3R)-3-benzyl-4-({1-[trans-2-(3-methoxypropoxy)cycloheptyl]-5-phenyl-1H-imidazol-4-yl}carbonyl)piperazine-1-carboxylate

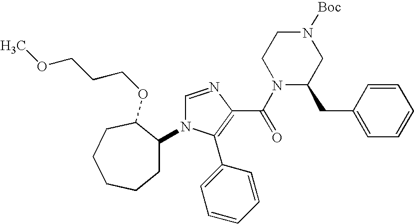

A mixture of tert-butyl (3R)-3-benzyl-4-({1-[trans-2-hydroxycycloheptyl]-5-phenyl-1H-imidazol-4-yl}carbonyl)piperazine-1-carboxylate (105 mg), sodium hydride (60% in oil, 15 mg) and THF (5 ml) was stirred at room temperature for 1 hr, and ice-cooled. To the reaction mixture was added 1-bromo-3-methoxypropane (45 mg) under ice-cooling, and the mixture was stirred at room temperature for 2 hr, and then at 65° C. for 12 hr. The mixture was poured into saturated aqueous sodium hydrogen carbonate, and the mixture was extracted with ethyl acetate. The extract was washed successively with water and saturated brine, and dried over anhydrous magnesium sulfate, and the solvent was evaporated under reduced pressure. The residue was subjected to silica gel column chromatography, and the fraction eluted with ethyl acetate-hexane (1:4-1:0) was concentrated under reduced pressure to give the object compound (40 mg).

MS (ESI+, m/e) 631 (M+1)

In the same manner as in Reference Example 556, the following compound (Reference Example 557) was obtained.

### Reference Example 557

### tert-Butyl (3R)-3-benzyl-4-({1-[trans-2-(2-methoxyethoxy)cycloheptyl]-5-phenyl-1H-imidazol-4-yl}carbonyl)piperazine-1-carboxylate

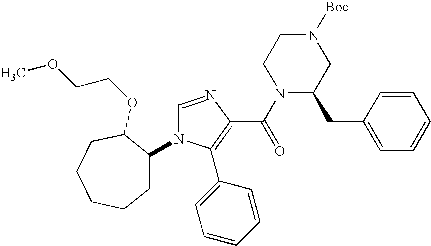

MS (ESI+, m/e) 617 (M+1)

### Reference Example 558

### tert-Butyl (3R)-3-benzyl-4-{[1-((1S,2S)-2-{[(ethylamino)carbonyl]oxy}cyclohexyl)-5-phenyl-1H-imidazol-4-yl]carbonyl}piperazine-1-carboxylate

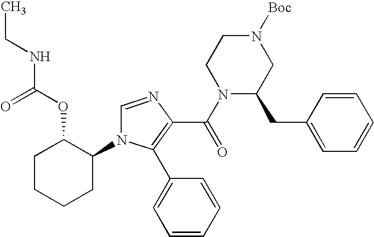

tert-Butyl (3R)-3-benzyl-4-({1-[(1S,2S)-2-hydroxycyclohexyl]-5-phenyl-1H-imidazol-4-yl}carbonyl)piperazine-1-carboxylate (163 mg) and DMAP (220 mg) were dissolved in THF (5 ml), and the solution was ice-cooled. 4-Nitrophenyl chloroformate (182 mg) was added, and the mixture was stirred at 0° C. for 1 hr. To the reaction mixture was added ethylamine (1M THF solution, 2 ml), and the mixture was stirred at room temperature for 1 hr. The reaction mixture was poured into saturated aqueous sodium hydrogen carbonate, and the mixture was extracted with ethyl acetate. The extract was washed with saturated brine, dried over anhydrous magnesium sulfate, and concentrated under reduced pressure. The residue was subjected to basic silica gel column chromatography, and the fraction eluted with ethyl acetate-methanol (9:1) was concentrated under reduced pressure to give the object compound (190 mg) as an amorphous solid.

MS (ESI+, m/e) 616 (M+1)

In the same manner as in Reference Example 558, the following compounds (Reference Examples 559-560) were obtained.

### Reference Example 559

### tert-Butyl (3R)-3-benzyl-4-({1-[(1S,2S)-2-({[(ethyl)(methyl)amino]carbonyl}oxy)cyclohexyl]-5-phenyl-1H-imidazol-4-yl}carbonyl)piperazine-1-carboxylate

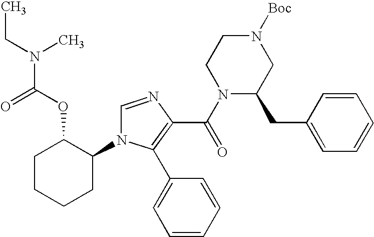

MS (ESI+, m/e) 630 (M+1)

### Reference Example 560

### tert-Butyl (3R)-3-benzyl-4-[(1-{(1S,2S)-2-[({(methyl)[2-(methylsulfonyl)ethyl]amino}carbonyl)oxy]cyclohexyl}-5-phenyl-1H-imidazol-4-yl)carbonyl]piperazine-1-carboxylate

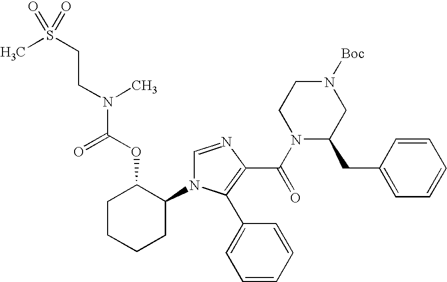

MS (ESI+, m/e) 708 (M+1)

### Reference Example 561

### tert-Butyl (3R)-3-benzyl-4-({1-[trans-2-({[(2-furylmethyl)amino]carbonyl}oxy)cycloheptyl]-5-phenyl-1H-imidazol-4-yl}carbonyl)piperazine-1-carboxylate

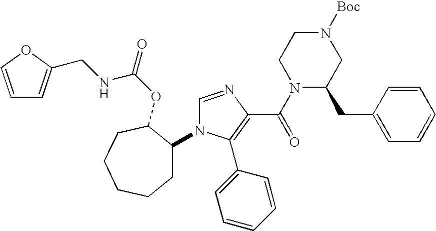

A mixture of tert-butyl (3R)-3-benzyl-4-({1-[trans-2-hydroxycycloheptyl]-5-phenyl-1H-imidazol-4-yl}carbonyl)piperazine-1-carboxylate (137 mg), 4-nitrophenyl chloroformate (75 mg), DMAP (100 mg) and THF (3 ml) was stirred at room temperature for 1 hr, and furfurylamine (110 mg) was added thereto. The reaction mixture was further stirred at room temperature for 3 days, and poured into saturated aqueous sodium hydrogen carbonate, and the mixture was extracted with ethyl acetate. The extract was washed successively with aqueous citric acid solution and saturated brine, and dried over anhydrous magnesium sulfate, and the solvent was evaporated under reduced pressure. The residue was subjected to silica gel column chromatography, and the fraction eluted with ethyl acetate-hexane (2:3-1:0) was concentrated under reduced pressure to give the object compound (115 mg).

MS (ESI+, m/e) 682 (M+1)

### Reference Example 562

### tert-Butyl (3R)-4-({1-[(1S,2R)-2-aminocyclohexyl]-5-phenyl-1H-imidazol-4-yl}carbonyl)-3-benzylpiperazine-1-carboxylate

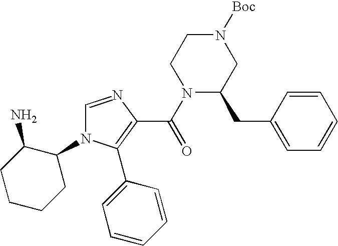

tert-Butyl (3R)-4-({1-[(1S,2R)-2-azidocyclohexyl]-5-phenyl-1H-imidazol-4-yl}carbonyl)-3-benzylpiperazine-1-carboxylate (2.5 g) was dissolved in methanol (25 ml), 10% palladium-carbon (50% containing water, 800 mg) was added thereto, and the mixture was subjected to catalytic reduction at ambient temperature and normal pressure for 15 hr. The catalyst was filtered off, and the filtrate was concentrated under reduced pressure to give the object compound (2.26 g) as an amorphous solid.

MS (ESI+, m/e) 544 (M+1)

### Reference Example 563

### tert-Butyl (3R)-3-benzyl-4-[(1-{(1S,2R)-2-[(cyclopropylmethyl)amino]cyclohexyl}-5-phenyl-1H-imidazol-4-yl)carbonyl]piperazine-1-carboxylate

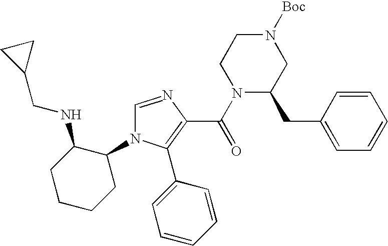

tert-Butyl (3R)-4-({1-[(1S,2R)-2-aminocyclohexyl]-5-phenyl-1H-imidazol-4-yl}carbonyl)-3-benzylpiperazine-1-carboxylate (217 mg) and cyclopropanecarbaldehyde (28 mg) were dissolved in dichloroethane (2 ml), and acetic acid (24 mg) and sodium triacetoxyborohydride (110 mg) were added. The mixture was stirred at room temperature for 5 hr, and neutralized with 6% aqueous sodium bicarbonate. The organic layer was washed successively with water and saturated brine, and dried over anhydrous magnesium sulfate, and the solvent was evaporated under reduced pressure. The residue was subjected to silica gel column chromatography, and the fraction eluted with ethyl acetate-methanol (1:0-9:1) was concentrated under reduced pressure to give the object compound (150 mg) as an amorphous solid.

MS (ESI+, m/e) 598 (M+1)

In the same manner as in Reference Example 563, the following compound (Reference Example 564) was obtained.

### Reference Example 564

### tert-Butyl (3R)-3-benzyl-4-[(1-{(1S,2R)-2-[bis(cyclopropylmethyl)amino]cyclohexyl}-5-phenyl-1H-imidazol-4-yl)carbonyl]piperazine-1-carboxylate

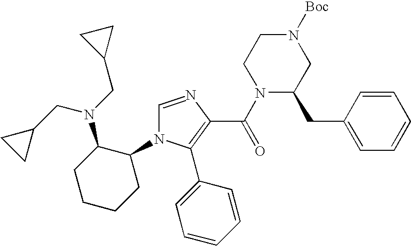

MS (ESI+, m/e) 652 (M+1)

### Reference Example 565

### tert-Butyl (3R)-3-benzyl-4-[(1-{(1S,2R)-2-[(cyclopropylcarbonyl)amino]cyclohexyl}-5-phenyl-1H-imidazol-4-yl)carbonyl]piperazine-1-carboxylate

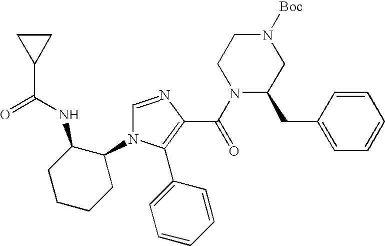

tert-Butyl (3R)-4-({1-[(1S,2R)-2-aminocyclohexyl]-5-phenyl-1H-imidazol-4-yl}carbonyl)-3-benzylpiperazine-1-carboxylate (217 mg) and triethylamine (60 mg) were dissolved in dichloromethane (3 ml), the solution was ice-cooled, and cyclopropanecarbonyl chloride (52 mg) was added. The mixture was stirred at 0° C. for 30 min, and neutralized with 6% aqueous sodium bicarbonate (2 ml). The organic layer was washed successively with water and saturated brine, and dried over anhydrous magnesium sulfate, and the solvent was evaporated under reduced pressure. The residue was subjected to silica gel column chromatography, and the fraction eluted with ethyl acetate-methanol (1:0-9:1) was concentrated under reduced pressure to give the object compound (203 mg) as an amorphous solid.

MS (ESI+, m/e) 612 (M+1)

In the same manner as in Reference Example 565, the following compounds (Reference Examples 566-567) were obtained.

### Reference Example 566

### tert-Butyl (3R)-3-benzyl-4-[(1-{(1S,2R)-2-[(cyclopropylsulfonyl)amino]cyclohexyl}-5-phenyl-1H-imidazol-4-yl)carbonyl]piperazine-1-carboxylate

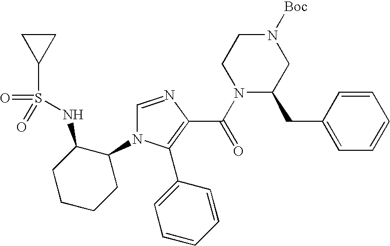

MS (ESI+, m/e) 648 (M+1)

### Reference Example 567

### tert-Butyl (3R)-3-benzyl-4-({1-[(1S,2R)-2-(butyrylamino)cyclohexyl]-5-phenyl-1H-imidazol-4-yl}carbonyl)piperazine-1-carboxylate

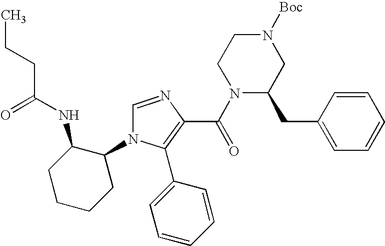

MS (ESI+, m/e) 614 (M+1)

### Reference Example 568

### tert-Butyl (3R)-3-benzyl-4-{[1-((1S,2R)-2-{[(ethylamino)carbonyl]amino}cyclohexyl)-5-phenyl-1H-imidazol-4-yl]carbonyl}piperazine-1-carboxylate

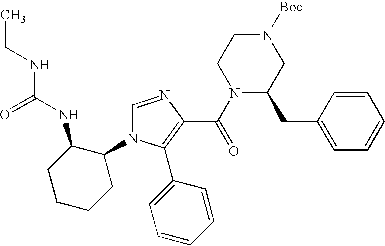

To a solution of tert-butyl (3R)-4-({1-[(1S,2R)-2-aminocyclohexyl]-5-phenyl-1H-imidazol-4-yl}carbonyl)-3-benzylpiperazine-1-carboxylate (217 mg) in dichloromethane (3 ml) were added ethyl isocyanate (36 mg) and triethylamine (1 drop) at room temperature. The mixture was stirred at room temperature for 2 hr, and the solvent was concentrated under reduced pressure. The residue was subjected to silica gel column chromatography, and the fraction eluted with ethyl acetate-methanol (1:0-9:1) was concentrated under reduced pressure to give the object compound (175 mg) as an amorphous solid.

MS (ESI+, m/e) 615 (M+1)

### Reference Example 569

### Methyl [(1S,2S)-2-(4-{[(2R)-2,4-dibenzylpiperazin-1-yl]carbonyl}-5-phenyl-1H-imidazol-1-yl)cyclohexyl]carbamate

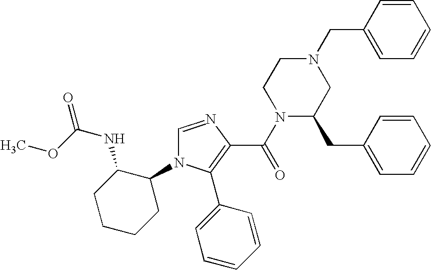

(1S,2S)-2-(4-{[(2R)-2,4-Dibenzylpiperazin-1-yl]carbonyl}-5-phenyl-1H-imidazol-1-yl)cyclohexanamine (160 mg) and triethylamine (36 mg) were dissolved in dichloromethane (2 ml), and the solution was ice-cooled. Methyl chloroformate (28 mg) was added thereto, and the mixture was stirred at 0° C. for 2 hr, and concentrated under reduced pressure. Ethyl acetate was added to the residue, and the mixture was washed successively with water and saturated brine, dried over anhydrous magnesium sulfate, and concentrated under reduced pressure. The residue was subjected to silica gel column chromatography, and the fraction eluted with ethyl acetate-hexane (3:7-7:3) was concentrated under reduced pressure to give the object compound (100 mg) as an amorphous solid.

MS (ESI+, m/e) 592 (M+1)

### Reference Example 570

### Ethyl [(1S,2S)-2-(4-{[(2R)-2,4-dibenzylpiperazin-1-yl]carbonyl}-5-phenyl-1H-imidazol-1-yl)cyclohexyl]carbamate

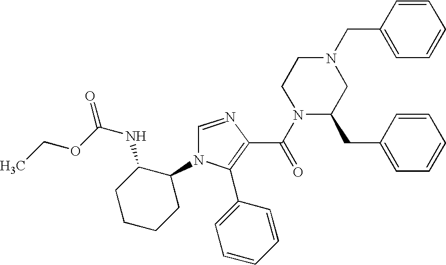

(1S,2S)-2-(4-{[(2R)-2,4-Dibenzylpiperazin-1-yl]carbonyl}-5-phenyl-1H-imidazol-1-yl)cyclohexanamine (300 mg) and triethylamine (85 mg) were dissolved in dichloromethane (5 ml), and the solution was ice-cooled. Ethyl chloroformate (73 mg) was added thereto, and the mixture was stirred at 0° C. for 2 hr, and concentrated under reduced pressure. Ethyl acetate was added to the residue, and the mixture was washed successively with water and saturated brine, dried over anhydrous magnesium sulfate, and concentrated under reduced pressure. The residue was subjected to silica gel column chromatography, and the fraction eluted with ethyl acetate-hexane (3:7-7:3) was concentrated under reduced pressure to give the object compound (260 mg) as an amorphous solid.

MS (ESI+, m/e) 606 (M+1)

In the same manner as in Reference Example 570, the following compounds (Reference Examples 571-574) were obtained.

### Reference Example 571

### Isopropyl [(1S,2S)-2-(4-{[(2R)-2,4-dibenzylpiperazin-1-yl]carbonyl}-5-phenyl-1H-imidazol-1-yl)cyclohexyl]carbamate

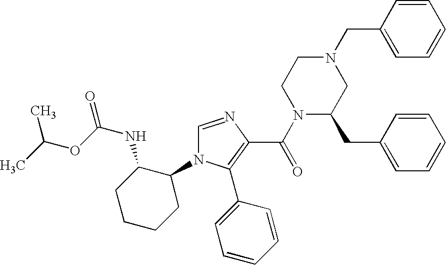

MS (ESI+, m/e) 620 (M+1)

### Reference Example 572

### Isobutyl [(1S,2S)-2-(4-{[(2R)-2,4-dibenzylpiperazin-1-yl]carbonyl}-5-phenyl-1H-imidazol-1-yl)cyclohexyl]carbamate

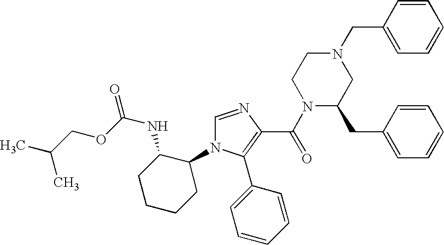

MS (ESI+, m/e) 634 (M+1)

### Reference Example 573

### 2-Methoxyethyl [(1S,2S)-2-(4-{[(2R)-2,4-dibenzylpiperazin-1-yl]carbonyl}-5-phenyl-1H-imidazol-1-yl)cyclohexyl]carbamate

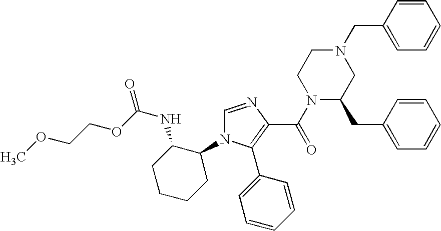

MS (ESI+, m/e) 636 (M+1)

### Reference Example 574

### 2-Chloroethyl [(1S,2S)-2-(4-{[(2R)-2,4-dibenzylpiperazin-1-yl]carbonyl}-5-phenyl-1H-imidazol-1-yl)cyclohexyl]carbamate

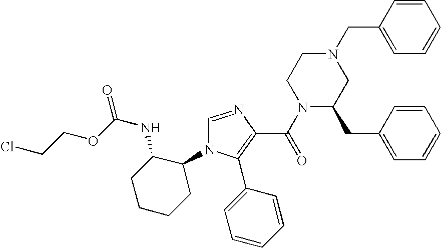

MS (ESI+, m/e) 641 (M+1)

### Reference Example 575

### 3-[(1S,2S)-2-(4-{[(2R)-2,4-Dibenzylpiperazin-1-yl]carbonyl}-5-phenyl-1H-imidazol-1-yl)cyclohexyl]-1,3-oxazolidin-2-one

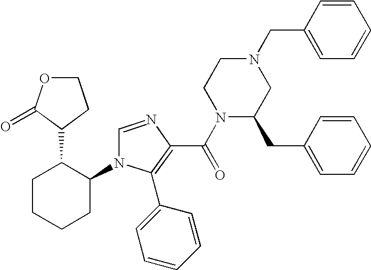

2-Chloroethyl [(1S,2S)-2-(4-{[(2R)-2,4-dibenzylpiperazin-1-yl]carbonyl}-5-phenyl-1H-imidazol-1-yl)cyclohexyl]carbamate (192 mg) was dissolved in THF (3 ml), sodium hydride (60% in oil, 14 mg) was added thereto, and the mixture was stirred at room temperature for 2 hr, and concentrated under reduced pressure. Ethyl acetate was added to the residue, and the mixture was washed successively with water and saturated brine, dried over anhydrous magnesium sulfate, and concentrated under reduced pressure. The residue was subjected to silica gel column chromatography, and the fraction eluted with ethyl acetate-methanol (1:0-9:1) was concentrated under reduced pressure to give the object compound (120 mg) as an amorphous solid.

MS (ESI+, m/e) 604 (M+1)

### Reference Example 576

### tert-Butyl (3R)-3-benzyl-4-[(1-{(1S,2S)-2-[(ethoxycarbonyl)(methyl)amino]cyclohexyl}-5-phenyl-1H-imidazol-4-yl)carbonyl]piperazine-1-carboxylate

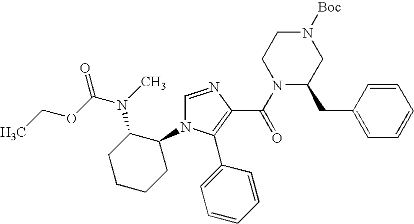

tert-Butyl (3R)-3-benzyl-4-[(1-{(1S,2S)-2-[(ethoxycarbonyl)amino]cyclohexyl}-5-phenyl-1H-imidazol-4-yl)carbonyl]piperazine-1-carboxylate (185 mg) was dissolved in DMF (2 ml), sodium hydride (60% in oil, 24 mg) was added thereto, and the mixture was stirred at room temperature for 30 min. After stirring, methyl iodide (85 mg) was added thereto, and the mixture was further stirred at room temperature for 15 hr, and concentrated under reduced pressure. Ethyl acetate was added to the residue, and the mixture was washed successively with water and saturated brine, dried over anhydrous magnesium sulfate, and concentrated under reduced pressure. The residue was subjected to basic silica gel column chromatography, and the fraction eluted with ethyl acetate-hexane (3:7-7:3) was concentrated under reduced pressure to give the object compound (145 mg) as an amorphous solid.

MS (ESI+, m/e) 630 (M+1)

In the same manner as in Reference Example 576, the following compound (Reference Example 577) was obtained.

### Reference Example 577

### tert-Butyl (3R)-3-benzyl-4-[(1-{(1S,2S)-2-[(ethoxycarbonyl)(3-methoxypropyl)amino]cyclohexyl}-5-phenyl-1H-imidazol-4-yl)carbonyl]piperazine-1-carboxylate

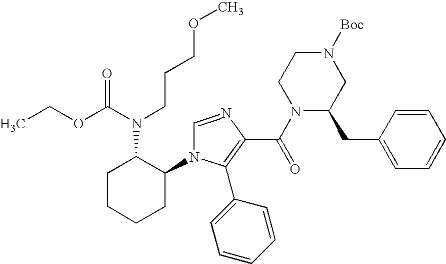

MS (ESI+, m/e) 688 (M+1)

### Reference Example 578

### tert-Butyl (3R)-3-benzyl-4-({1-[(1S)-2-oxocyclohexyl]-5-phenyl-1H-imidazol-4-yl}carbonyl)piperazine-1-carboxylate

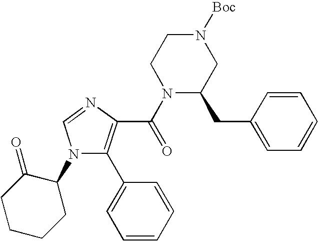

tert-Butyl (3R)-3-benzyl-4-({1-[(1S,2S)-2-hydroxycyclohexyl]-5-phenyl-1H-imidazol-4-yl}carbonyl)piperazine-1-carboxylate (4.2 g) was dissolved in dichloromethane (60 ml). A solution of Dess-Martin reagent (3.9 g) in dichloromethane (60 ml) was added, and the mixture was stirred at room temperature for 3 hr. The reaction mixture was poured into water, and the mixture was extracted with chloroform. The extract was concentrated under reduced pressure, and the residue was dissolved in ethyl acetate-THF. 10% Aqueous sodium thiosulfate solution was added thereto, and the mixture was stirred at room temperature for 30 min. The organic layer was washed successively with saturated aqueous sodium hydrogen carbonate and saturated brine, and dried over anhydrous sodium sulfate, and the solvent was evaporated under reduced pressure. To the residue was added ethyl acetate, and the precipitated crystals were collected by filtration to give the object compound (2.35 g). The second crystals (1.37 g) of the object compound were obtained from the mother liquor. The yield of the obtained object compound was 3.72 g in total.

MS (ESI+, m/e) 543 (M+1)

In the same manner as in Reference Example 578, the following compounds (Reference Examples 579-580) were obtained.

### Reference Example 579

### tert-Butyl (3R)-3-benzyl-4-({1-[(1R)-2-oxocyclohexyl]-5-phenyl-1H-imidazol-4-yl}carbonyl)piperazine-1-carboxylate

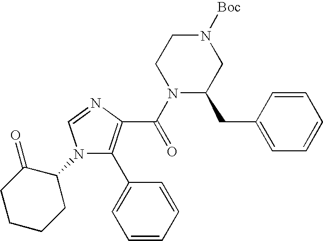

MS (ESI+, m/e) 543 (M+1)

### Reference Example 580

### tert-Butyl (3R)-3-benzyl-4-{[1-(2-oxocyclohexyl)-5-phenyl-1H-imidazol-4-yl]carbonyl}piperazine-1-carboxylate

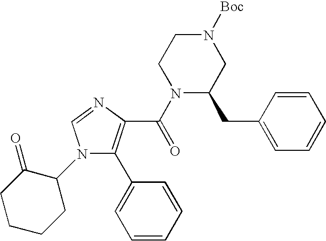

MS (ESI+, m/e) 543 (M+1)

### Reference Example 581

### tert-Butyl (3R)-3-benzyl-4-{[1-(2-butyl-2-hydroxycyclohexyl)-5-phenyl-1H-imidazol-4-yl]carbonyl}piperazine-1-carboxylate

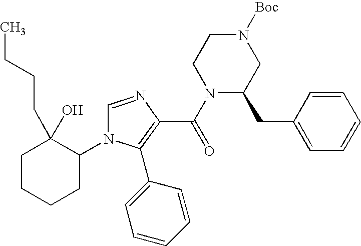

tert-Butyl (3R)-3-benzyl-4-{[1-(2-oxocyclohexyl)-5-phenyl-1H-imidazol-4-yl]carbonyl}piperazine-1-carboxylate (150 mg) was dissolved in THF (5 ml), and the solution was cooled to −78° C. n-Butylmagnesium chloride (2M THF solution, 560 μl) was added thereto, and the mixture was stirred at −78° C. for 1.5 hr. To the reaction mixture was added saturated aqueous ammonium chloride solution, and the mixture was extracted with ethyl acetate. The extract was washed with saturated brine, and dried over anhydrous sodium sulfate, and the solvent was evaporated under reduced pressure. The residue was subjected to silica gel column chromatography, and the fraction eluted with ethyl acetate-hexane (3:7-7:3) was concentrated under reduced pressure to give the object compound (36 mg) as an amorphous solid.

MS (ESI+, m/e) 601 (M+1)

### Reference Example 582

### tert-Butyl (3R)-3-benzyl-4-{[1-(2-hydroxy-2-methylcyclohexyl)-5-phenyl-1H-imidazol-4-yl]carbonyl}piperazine-1-carboxylate

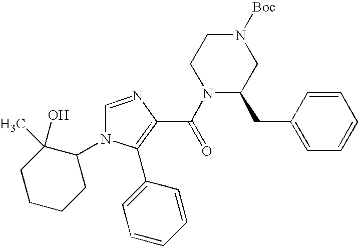

tert-Butyl (3R)-3-benzyl-4-{[1-(2-oxocyclohexyl)-5-phenyl-1H-imidazol-4-yl]carbonyl}piperazine-1-carboxylate (163 mg) was dissolved in THF (2 ml), and the solution was ice-cooled. Methylmagnesium bromide (3M diethyl ether solution, 300 μl) was added, and the mixture was stirred at 0° C. for 30 min. To the reaction mixture was added saturated aqueous ammonium chloride solution, and the mixture was extracted with ethyl acetate. The extract was washed with saturated brine, and dried over anhydrous sodium sulfate, and the solvent was evaporated under reduced pressure. The residue was subjected to silica gel column chromatography, and the fraction eluted with ethyl acetate-hexane (3:7-7:3) was concentrated under reduced pressure to give the object compound (110 mg) as an amorphous solid.

MS (ESI+, m/e) 559 (M+1)

### Reference Example 583

### tert-Butyl (3R)-3-benzyl-4-({1-[2-hydroxy-2-(trifluoromethyl)cyclohexyl]-5-phenyl-1H-imidazol-4-yl}carbonyl)piperazine-1-carboxylate

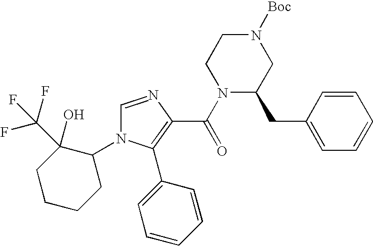

tert-Butyl (3R)-3-benzyl-4-{[1-(2-oxocyclohexyl)-5-phenyl-1H-imidazol-4-yl]carbonyl}piperazine-1-carboxylate (150 mg) and trimethyl(trifluoromethyl)silane (79 mg) were dissolved in THF (2 ml), TBAF (several mg) was added, and the mixture was stirred at room temperature for 15 hr, and concentrated under reduced pressure. The residue was subjected to silica gel column chromatography, and the fraction eluted with ethyl acetate-hexane (3:7-7:3) was concentrated under reduced pressure to give the object compound (38 mg) as an amorphous solid.

MS (ESI+, m/e) 613 (M+1)

### Reference Example 584

### tert-Butyl (3R)-3-benzyl-4-({1-[(1S)-2-(2-ethoxy-2-oxoethyl)-2-hydroxycyclohexyl]-5-phenyl-1H-imidazol-4-yl}carbonyl)piperazine-1-carboxylate

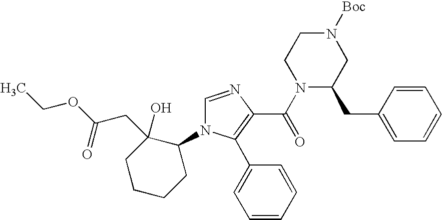

tert-Butyl (3R)-3-benzyl-4-({1-[(1S)-2-oxocyclohexyl]-5-phenyl-1H-imidazol-4-yl}carbonyl)piperazine-1-carboxylate (150 mg) was dissolved in THF (5 ml), bromo(2-ethoxy-2-oxoethyl)zinc (0.5M THF solution, 4 ml) was added thereto at room temperature, and the mixture was stirred at 60° C. for 2 hr. The reaction mixture was poured into water, and the mixture was extracted with ethyl acetate. The extract was dried over anhydrous sodium sulfate, and the solvent was evaporated under reduced pressure. The residue was subjected to silica gel column chromatography, and the fraction eluted with ethyl acetate-hexane (1:1) was concentrated under reduced pressure to give the object compound (140 mg) as an amorphous solid.

MS (ESI+, m/e) 631 (M+1)

### Reference Example 585

### [(2S)-2-(4-{[(2R)-2-Benzyl-4-(tert-butoxycarbonyl)piperazin-1-yl]carbonyl}-5-phenyl-1H-imidazol-1-yl)-1-hydroxycyclohexyl]acetic acid

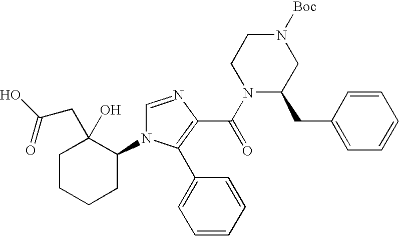

tert-Butyl (3R)-3-benzyl-4-({1-[(1S)-2-(2-ethoxy-2-oxoethyl)-2-hydroxycyclohexyl]-5-phenyl-1H-imidazol-4-yl}carbonyl)piperazine-1-carboxylate (300 mg) was dissolved in ethanol (2 ml), and 1N aqueous sodium hydroxide solution (4 ml) was added. The mixture was stirred at room temperature for 1 hr, and the solvent was evaporated under reduced pressure. The residual aqueous solution was washed with ethyl acetate, and neutralized with 10% aqueous citric acid solution. This was extracted with ethyl acetate, the extract was washed with saturated brine, and dried over anhydrous sodium sulfate, and the solvent was evaporated under reduced pressure to give the object compound (280 mg).

MS (ESI+, m/e) 603 (M+1)

### Reference Example 586

### tert-Butyl (3R)-3-benzyl-4-[(1-{(1S)-2-hydroxy-2-[2-(methylamino)-2-oxoethyl]cyclohexyl}-5-phenyl-1H-imidazol-4-yl)carbonyl]piperazine-1-carboxylate

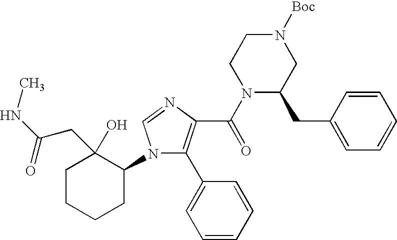

A solution of [(2S)-2-(4-{[(2R)-2-benzyl-4-(tert-butoxycarbonyl)piperazin-1-yl]carbonyl}-5-phenyl-1H-imidazol-1-yl)-1-hydroxycyclohexyl]acetic acid (100 mg), methylamine (2M THF solution, 91 μl), WSC.HCl (41 mg) and HOBt (30 mg) in DMF (2 ml) was stirred at room temperature for 12 hr, and poured into saturated aqueous sodium hydrogen carbonate, and the mixture was extracted with ethyl acetate. The extract was washed with saturated brine, and dried over anhydrous sodium sulfate, and the solvent was evaporated under reduced pressure. The residue was subjected to silica gel column chromatography, and the fraction eluted with ethyl acetate-methanol (4:1) was concentrated under reduced pressure to give the object compound (70 mg) as an amorphous solid.

MS (ESI+, m/e) 616 (M+1)

In the same manner as in Reference Example 586, the following compounds (Reference Examples 587-588) were obtained.

### Reference Example 587

### tert-Butyl (3R)-3-benzyl-4-[(1-{(1S)-2-[2-(dimethylamino)-2-oxoethyl]-2-hydroxycyclohexyl}-5-phenyl-1H-imidazol-4-yl)carbonyl]piperazine-1-carboxylate

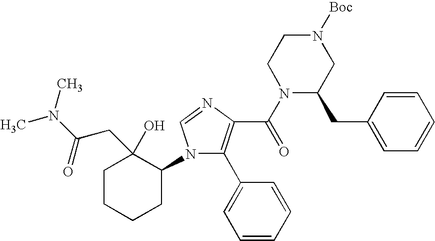

MS (ESI+, m/e) 630 (M+1)

### Reference Example 588

### tert-Butyl (3R)-3-benzyl-4-({1-[(1S)-2-{2-[(2-furylmethyl)amino]-2-oxoethyl}-2-hydroxycyclohexyl]-5-phenyl-1H-imidazol-4-yl}carbonyl)piperazine-1-carboxylate

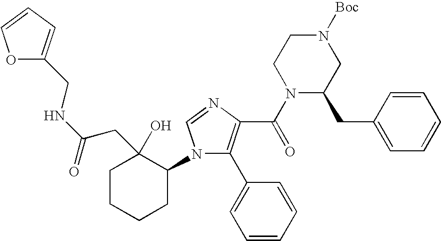

MS (ESI+, m/e) 682 (M+1)

### Reference Example 589

### tert-Butyl (3R)-3-benzyl-4-({1-[(1S)-2-hydroxy-2-(2-hydroxyethyl)cyclohexyl]-5-phenyl-1H-imidazol-4-yl}carbonyl)piperazine-1-carboxylate

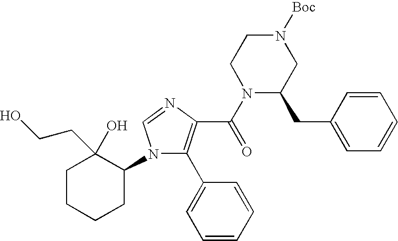

Sodium borohydride (862 mg) was suspended in ethanol (9 ml), and the suspension was ice-cooled. Calcium chloride (1.23 g) was added over 10 min, and the mixture was stirred at 0° C. for 30 min. A solution of tert-butyl (3R)-3-benzyl-4-({1-[(1S)-2-(2-ethoxy-2-oxoethyl)-2-hydroxycyclohexyl]-5-phenyl-1H-imidazol-4-yl}carbonyl)piperazine-1-carboxylate (900 mg) in THF (9 ml) was added thereto over 20 min, and the mixture was stirred at 0° C. for 2 hr, and then at room temperature for 2 hr. Water (20 ml) was slowly added. This was extracted with ethyl acetate, the extract was washed successively with water and saturated brine, and dried over anhydrous magnesium sulfate, and the solvent was evaporated under reduced pressure. The residue was subjected to silica gel column chromatography, and the fraction eluted with ethyl acetate-hexane (1:1) was concentrated under reduced pressure to give the object compound (830 mg) as an amorphous solid.

MS (ESI+, m/e) 589 (M+1)

### Reference Example 590

### tert-Butyl (3R)-3-benzyl-4-({1-[(1S)-2-hydroxy-2-(2-oxoethyl)cyclohexyl]-5-phenyl-1H-imidazol-4-yl}carbonyl)piperazine-1-carboxylate

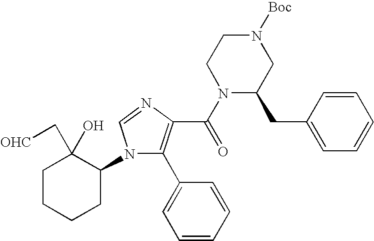

tert-Butyl (3R)-3-benzyl-4-({1-[(1S)-2-hydroxy-2-(2-hydroxyethyl)cyclohexyl]-5-phenyl-1H-imidazol-4-yl}carbonyl)piperazine-1-carboxylate (530 mg) was dissolved in dichloromethane (7 ml). A solution of Dess-Martin reagent (460 mg) in dichloromethane (5 ml) was added, and the mixture was stirred at room temperature for 2 hr. The reaction mixture was diluted with chloroform (30 ml), 10% aqueous sodium thiosulfate solution was added, and the mixture was stirred for 30 min. The organic layer was separated, washed successively with saturated aqueous sodium hydrogen carbonate and saturated brine, and dried over anhydrous sodium sulfate, and the solvent was evaporated under reduced pressure to give the object compound (517 mg).

MS (ESI+, m/e) 586 (M+1)

### Reference Example 591

### tert-Butyl (3R)-3-benzyl-4-[(1-{(1S)-2-[2-(benzylamino)ethyl]-2-hydroxycyclohexyl}-5-phenyl-1H-imidazol-4-yl)carbonyl]piperazine-1-carboxylate

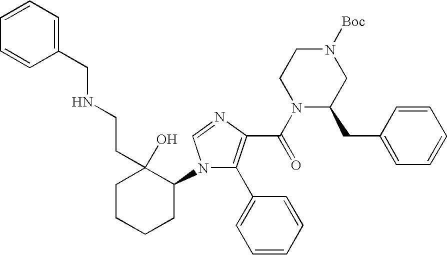

tert-Butyl (3R)-3-benzyl-4-({1-[(1S)-2-hydroxy-2-(2-oxoethyl)cyclohexyl]-5-phenyl-1H-imidazol-4-yl}carbonyl)piperazine-1-carboxylate (300 mg) was dissolved in DMF-dichloromethane (1:2, 3 ml), benzylamine (134 μl) and acetic acid (2 drops) were added, and the mixture was stirred at room temperature for 15 min. Sodium triacetoxyborohydride (163 mg) was added thereto, and the mixture was further stirred at room temperature for 12 hr. To the reaction mixture was added ethyl acetate (3 ml) over 15 min, and the mixture was poured into saturated aqueous sodium hydrogen carbonate, and extracted with ethyl acetate. The extract was washed with saturated brine, and dried over anhydrous sodium sulfate, and the solvent was evaporated under reduced pressure. The residue was subjected to silica gel column chromatography, and the fraction eluted with chloroform-methanol (9:1) was concentrated under reduced pressure to give the object compound (85 mg) as an amorphous solid.

MS (ESI+, m/e) 678 (M+1)

### Reference Example 592

### tert-Butyl (3R)-4-({1-[(1S)-2-(2-aminoethyl)-2-hydroxycyclohexyl]-5-phenyl-1H-imidazol-4-yl}carbonyl)-3-benzylpiperazine-1-carboxylate

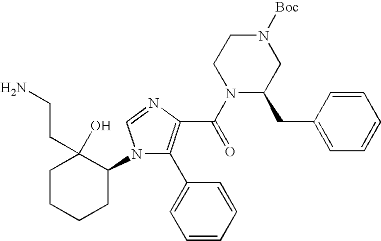

tert-Butyl (3R)-3-benzyl-4-[(1-{(1S)-2-[2-(benzylamino)ethyl]-2-hydroxycyclohexyl}-5-phenyl-1H-imidazol-4-yl)carbonyl]piperazine-1-carboxylate (100 mg) was dissolved in methanol (3 ml), 20% palladium hydroxide-carbon (50% containing water, 30 mg) was added thereto, and the mixture was subjected to catalytic reduction at ambient temperature and normal pressure for 12 hr. The catalyst was filtered off, and the filtrate was concentrated under reduced pressure. The residue was subjected to silica gel column chromatography, and the fraction eluted with ethyl acetate-methanol (1:1) was concentrated under reduced pressure to give the object compound (25 mg) as an amorphous solid.

MS (ESI+, m/e) 588 (M+1)

### Reference Example 593

### tert-Butyl (3R)-4-({1-[(1S)-2-(2-acetamidoethyl)-2-hydroxycyclohexyl]-5-phenyl-1H-imidazol-4-yl}carbonyl)-3-benzylpiperazine-1-carboxylate

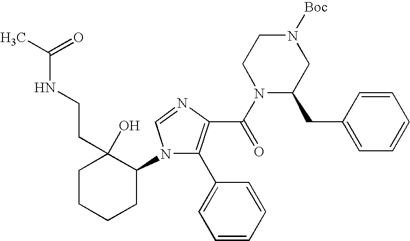

tert-Butyl (3R)-4-({1-[(1S)-2-(2-aminoethyl)-2-hydroxycyclohexyl]-5-phenyl-1H-imidazol-4-yl}carbonyl)-3-benzylpiperazine-1-carboxylate (150 mg) and triethylamine (13 mg) were dissolved in dichloromethane (3.5 ml), acetyl chloride (8 mg) was added thereto, and the mixture was stirred at room temperature for 1 hr. The reaction mixture was poured into aqueous sodium bicarbonate, and the mixture was extracted with ethyl acetate. The extract was washed with saturated brine, and dried over anhydrous sodium sulfate, and the solvent was evaporated under reduced pressure. The residue was subjected to silica gel column chromatography, and the fraction eluted with ethyl acetate-methanol (1:1) was concentrated under reduced pressure to give the object compound (41 mg) as an amorphous solid.

MS (ESI+, m/e) 630 (M+1)

### Reference Example 594

### tert-Butyl (3R)-3-benzyl-4-({1-[(1S)-2-methoxy-2-(2-methoxyethyl)cyclohexyl]-5-phenyl-1H-imidazol-4-yl}carbonyl)piperazine-1-carboxylate

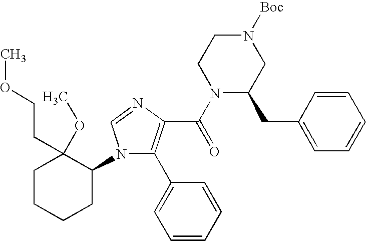

A mixture of tert-butyl (3R)-3-benzyl-4-({1-[(1S)-2-hydroxy-2-(2-hydroxyethyl)cyclohexyl]-5-phenyl-1H-imidazol-4-yl}carbonyl)piperazine-1-carboxylate (100 mg), silver oxide (44 mg), methyl iodide (0.150 ml) and dichloromethane (2 ml) was heated under reflux for 12 hr. The reaction mixture was subjected to silica gel column chromatography, and the fraction eluted with ethyl acetate was concentrated under reduced pressure to give the object compound (60 mg) as an amorphous solid.

MS (ESI+, m/e) 617 (M+1)

### Reference Example 595

### tert-Butyl (3R)-3-benzyl-4-({1-[(1S)-2-hydroxy-2-(2-methoxyethyl)cyclohexyl]-5-phenyl-1H-imidazol-4-yl}carbonyl)piperazine-1-carboxylate

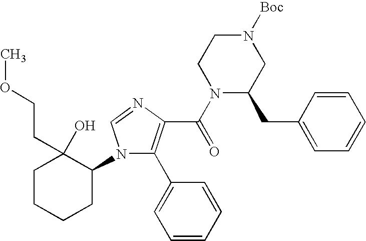

tert-Butyl (3R)-3-benzyl-4-({1-[(1S)-2-hydroxy-2-(2-hydroxyethyl)cyclohexyl]-5-phenyl-1H-imidazol-4-yl}carbonyl)piperazine-1-carboxylate (110 mg) was dissolved in DMF (2 ml), and the solution was ice-cooled. Sodium hydride (60% in oil, 18 mg) was added thereto, and the mixture was stirred at 0° C. for 30 min. Methyl iodide (14 μl) was added thereto, and the mixture was further stirred at 0° C. for 1 hr. The reaction mixture was poured into ice water, and the mixture was extracted with ethyl acetate. The extract was washed with saturated brine, and dried over anhydrous sodium sulfate, and the solvent was evaporated under reduced pressure. The residue was subjected to silica gel column chromatography, and the fraction eluted with ethyl acetate-methanol (9:1) was concentrated under reduced pressure to give the object compound (70 mg) as an amorphous solid.

MS (ESI+, m/e) 603 (M+1)

### Reference Example 596

### tert-Butyl (3R)-3-benzyl-4-({1-[(1S,2E)-2-(2-ethoxy-2-oxoethylidene)cyclohexyl]-5-phenyl-1H-imidazol-4-yl}carbonyl)piperazine-1-carboxylate

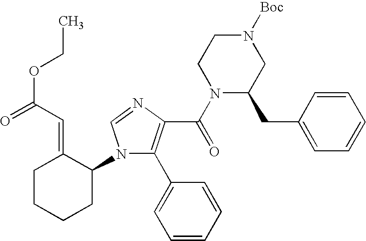

tert-Butyl (3R)-3-benzyl-4-({1-[(1S)-2-oxocyclohexyl]-5-phenyl-1H-imidazol-4-yl}carbonyl)piperazine-1-carboxylate (500 mg) and ethyl (diethoxyphosphoryl)acetate (227 mg) were dissolved in THF (5 ml), and the solution was ice-cooled. Sodium hydride (60% in oil) (55 mg) was added, and the mixture was stirred at room temperature for 12 hr. The reaction mixture was poured into saturated aqueous sodium hydrogen carbonate, and the mixture was extracted with ethyl acetate. The extract was washed with saturated brine, and dried over anhydrous sodium sulfate, and the solvent was evaporated under reduced pressure. The residue was subjected to silica gel column chromatography, and the fraction eluted with ethyl acetate was concentrated under reduced pressure to give the object compound (440 mg) as an amorphous solid.

MS (ESI+, m/e) 613 (M+1)

### Reference Example 597

### (2E)-[(2S)-2-(4-{[(2R)-2-Benzyl-4-(tert-butoxycarbonyl)piperazin-1-yl]carbonyl}-5-phenyl-1H-imidazol-1-yl)cyclohexylidene]acetic acid

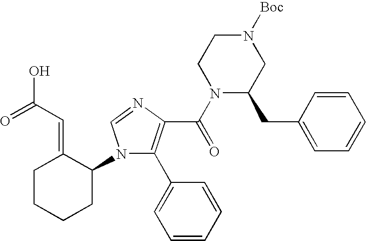

tert-Butyl (3R)-3-benzyl-4-({1-[(1S,2E)-2-(2-ethoxy-2-oxoethylidene)cyclohexyl]-5-phenyl-1H-imidazol-4-yl}carbonyl)piperazine-1-carboxylate (230 mg) was dissolved in ethanol (2 ml), 2N aqueous sodium hydroxide solution (2 ml) was added, and the mixture was stirred at room temperature for 1 hr, and neutralized with 10% aqueous citric acid solution. The solvent was evaporated under reduced pressure, and the residue was extracted with ethyl acetate-THF. The extract was dried over anhydrous sodium sulfate, and the solvent was evaporated under reduced pressure to give the object compound (220 mg).

MS (ESI+, m/e) 585 (M+1)

### Reference Example 598

### tert-Butyl (3R)-3-benzyl-4-[(1-{(1S,2E)-2-[2-oxo-2-(propylamino)ethylidene]cyclohexyl}-5-phenyl-1H-imidazol-4-yl)carbonyl]piperazine-1-carboxylate

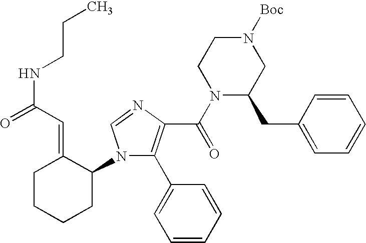

A solution of (2E)-[(2S)-2-(4-{[(2R)-2-benzyl-4-(tert-butoxycarbonyl)piperazin-1-yl]carbonyl}-5-phenyl-1H-imidazol-1-yl)cyclohexylidene]acetic acid (120 mg), propylamine (25 μl), WSC.HCl (59 mg) and HOBt (38 mg) in DMF (2 ml) was stirred at room temperature for 12 hr, and poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to basic silica gel column chromatography, and the fraction eluted with ethyl acetate was concentrated under reduced pressure to give the object compound (180 mg) as an oil.

MS (ESI+, m/e) 626 (M+1)

### Reference Example 599

### tert-Butyl (3R)-3-benzyl-4-{[1-(1-oxaspiro[2.5]oct-4-yl)-5-phenyl-1H-imidazol-4-yl]carbonyl}piperazine-1-carboxylate

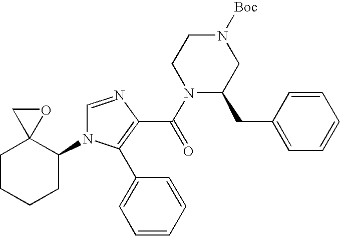

Trimethylsulfoxonium iodide (106 mg) was dissolved in DMSO (5 ml), sodium hydride (60% in oil, 19 mg) was added thereto, and the mixture was stirred at room temperature for 30 min. A solution of tert-butyl (3R)-3-benzyl-4-{[1-(2-oxocyclohexyl)-5-phenyl-1H-imidazol-4-yl]carbonyl}piperazine-1-carboxylate (400 mg) in DMSO (10 ml) was added thereto, and the mixture was stirred at room temperature for 30 min. The reaction mixture was poured into saturated aqueous ammonium chloride solution, and the mixture was extracted with ethyl acetate. The extract was washed with saturated brine, and dried over anhydrous sodium sulfate, and the solvent was evaporated under reduced pressure. The residue was subjected to basic silica gel column chromatography, and the fraction eluted with ethyl acetate was concentrated under reduced pressure to give the object compound (221 mg) as an amorphous solid.

MS (ESI+, m/e) 557 (M+1)

### Reference Example 600

### tert-Butyl (3R)-3-benzyl-4-({1-[2-hydroxy-2-(propoxymethyl)cyclohexyl]-5-phenyl-1H-imidazol-4-yl}carbonyl)piperazine-1-carboxylate

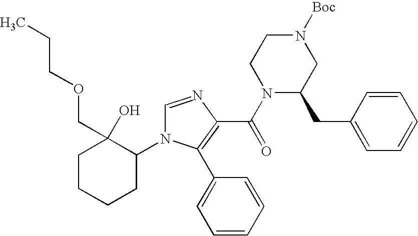

Sodium hydride (60% in oil) (60 mg) was suspended in DMF (3 ml), 1-propanol (135 μl) was added, and the mixture was stirred at room temperature for 30 min. tert-Butyl (3R)-3-benzyl-4-{[1-(1-oxaspiro[2.5]oct-4-yl)-5-phenyl-1H-imidazol-4-yl]carbonyl}piperazine-1-carboxylate (167 mg) was added thereto, and the mixture was stirred at 60° C. for 15 hr. To the reaction mixture was added aqueous sodium bicarbonate, and the mixture was extracted with ethyl acetate. The extract was washed with saturated brine, dried over anhydrous magnesium sulfate, and concentrated under reduced pressure. The residue was subjected to silica gel column chromatography, and the fraction eluted with ethyl acetate-methanol (9:1) was concentrated under reduced pressure to give the object compound (160 mg) as an amorphous solid.

MS (ESI+, m/e) 617 (M+1)

In the same manner as in Reference Example 600, the following compounds (Reference Examples 601-619) shown in Table 8-1-Table 8-3 were obtained.

TABLE 8-1

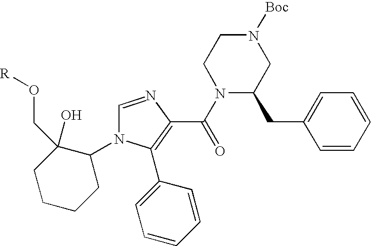

| Ref. Ex. No. | R | Compound | MS(ESI+) |
|---|---|---|---|
| 601 | 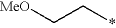 | tert-Butyl (3R)-3-benzyl-4-[(1-{2- hydroxy-2-[(2-methoxyethoxy)methyl] cyclohexyl}-5-phenyl-1H-imidazol-4- yl)carbonyl]piperazine-1-carboxylate | 633 |
| 602 | 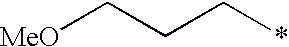 | tert-Butyl (3R)-3-benzyl-4-[(1-{2- hydroxy-2-[(3-methoxypropoxy)methyl] cyclohexyl}-5-phenyl-1H-imidazol-4- yl)carbonyl]piperazine-1-carboxylate | 647 |
| 603 | 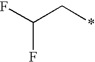 | tert-Butyl (3R)-3-benzyl-4-[(1-{2-[(2,2- difluoroethoxy)methyl]-2-hydroxy- cyclohexyl}-5-phenyl-1H-imidazol-4- yl)carbonyl]piperazine-1-carboxylate | 639 |
| 604 | 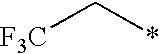 | tert-Butyl (3R)-3-benzyl-4-[(1-{2- hydroxy-2-[(2,2,2-trifluoroethoxy) methyl]cyclohexyl}-5-phenyl-1H- imidazol-4-yl)carbonyl]piperazine- 1-carboxylate | 657 |
| 605 | 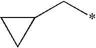 | tert-Butyl (3R)-3-benzyl-4-[(1-{2- [(cyclopropylmethoxylmethyl]-2- hydroxycyclohexyl}-5-phenyl-1H- imidazol-4-yl)carbonyl] piperazine-1-carboxylate | 629 |
| 606 | 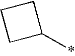 | tert-Butyl (3R)-3-benzyl-4-[(1-{2- [(cyclobutyloxy)methyl]-2- hydroxycyclohexyl-5-phenyl-1H- imidazol-4-yl)carbonyl] piperazine-1-carboxylate | 629 |
| 607 | 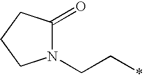 | tert-Butyl (3R)-3-benzyl-4-{[1-(2- hydroxy-2-{[2-(2-oxopyrrolidin-1- yl)ethoxy]methyl}cyclohexyl)-5- phenyl-1H-imidazol-4-yl]carbonyl} piperazine-1-carboxylate | 686 |
| 608 | 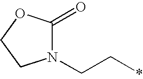 | tert-Butyl (3R)-3-benzyl-4-{[1-(2- hydroxy-2-{[2-(2-oxo-1,3-oxazolidin-3- yl)ethoxy]methyl}cyclohexyl)-5-phenyl- 1H-imidazol-4-yl]carbonyl}piperazine- 1-carboxylate | 688 |

TABLE 8-2

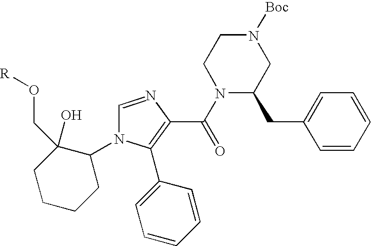

| Ref. Ex. No. | R | Compound | MS(ESI+) |
|---|---|---|---|
| 609 | 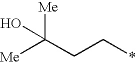 | tert-Butyl (3R)-3-benzyl-4-[(1-{2- hydroxy-2-[(3-hydroxy-3-methylbutoxy) methyl]cyclohexyl}-5-phenyl-1H-imidazol-4- yl)carbonyl]piperazine-1-carboxylate | 661 |
| 610 | 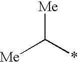 | tert-Butyl (3R)-3-benzyl-4-({1-[2- hydroxy-2-(isopropoxymethyl)cyclohexyl]- 5-phenyl-1H-imidazol-4-yl }carbonyl) piperazine-1-carboxylate | 617 |
| 611 | 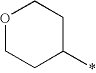 | tert-Butyl (3R)-3-benzyl-4-[(1-{2- hydroxy-2-[(tetrahydro-2H-pyran-4-yloxy) methyl]cyclohexyl}-5-phenyl-1H-imidazol- 4-yl)carbonyl]piperazine-1-carboxylate | 659 |
| 612 | 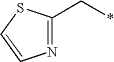 | tert-Butyl (3R)-3-benzyl-4-[(1-{2- hydroxy-2-[(1,3-thiazol-2-ylmethoxy)methyl] cyclohexyl}-5-phenyl-1H-imidazol-4-yl) carbonyl]piperazine-1-carboxylate | 672 |
| 613 | 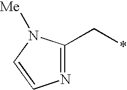 | tert-Butyl (3R)-3-benzyl-4-{[1-(2-hydroxy- 2-{[(1-methyl-1H-imidazol-2-yl)methoxy] methyl}cyclohexyl)-5-phenyl-1H-imidazol- 4-yl]carbonyl}piperazine-1-carboxylate | 669 |
| 614 | 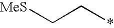 | tert-Butyl (3R)-3-benzyl-4-{[1-(2- hydroxy-2-{[2-(methylthio)ethoxy]methyl} cyclohexyl)-5-phenyl-1H-imidazol-4-yl] carbonyl}piperazine-1-carboxylate | 649 |
| 615 | 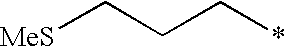 | tert-Butyl (3R)-3-benzyl-4-{[1-(2- hydroxy-2-{[3-(methylthio)propoxy]methyl} cyclohexyl)-5-phenyl-1H-imidazol-4-yl] carbonyl}piperazine-1-carboxylate | 663 |
| 616 | 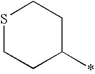 | tert-Butyl (3R)-3-benzyl-4-[(1-{2- hydroxy-2-[(tetrahydro-2H-thiopyran-4-yloxy) methyl]cyclohexyl}-5-phenyl-1H-imidazol-4- yl)carbonyl]piperazine-1-carboxylate | 675 |

TABLE 8-3

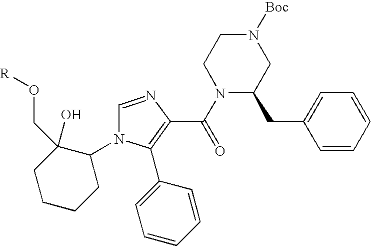

| Ref. | | | |
|---|---|---|---|
| Ex. No. | R | Compound | MS(ESI+) |
| 617 | 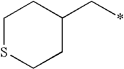 | tert-Butyl (3R)-3-benzyl-4- [(1-{2-hydroxy-2-[(tetra- hydro-2H-thiopyran-4-yl- methoxy)methyl]cyclohexyl}- 5-phenyl-1H-imidazol-4-yl) carbonyl]piperazine-1- carboxylate | 689 |
| 618 | 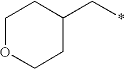 | tert-Butyl (3R)-3-benzyl-4- [(1-{2-hydroxy-2-[(tetra- hydro-2H-pyran-4-ylmethoxy) methyl]cyclohexyl}-5-phenyl- 1H-imidazol-4-yl)carbonyl] piperazine-1-carboxylate | 673 |
| 619 | 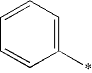 | tert-Butyl (3R)-3-benzyl-4- ({1-[2-hydroxy-2-(phenoxy- methyl)cyclohexyl]-5-phenyl- 1H-imidazol-4-yl}carbonyl) piperazine-1-carboxylate | 651 |

### Reference Example 620

### tert-Butyl (3R)-3-benzyl-4-[(1-{2-[(ethylamino)methyl]-2-hydroxycyclohexyl}-5-phenyl-1H-imidazol-4-yl)carbonyl]piperazine-1-carboxylate

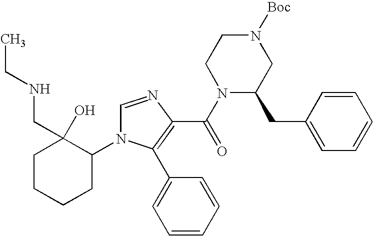

tert-Butyl (3R)-3-benzyl-4-{[1-(1-oxaspiro[2.5]oct-4-yl)-5-phenyl-1H-imidazol-4-yl]carbonyl}piperazine-1-carboxylate (240 mg) and ethylamine (2M THF solution, 650 μl) were dissolved in acetonitrile (3 ml), lithium perchlorate (92 mg) was added, and the mixture was reacted at 100° C. for 5 min using microwave reactor. The reaction mixture was concentrated under reduced pressure. The residue was subjected to basic silica gel column chromatography, and the fraction eluted with ethyl acetate-methanol (4:1) was concentrated under reduced pressure to give the object compound (220 mg) as an amorphous solid.

MS (ESI+, m/e) 602 (M+1)

In the same manner as in Reference Example 620, the following compounds (Reference Examples 621-622) were obtained.

### Reference Example 621

### tert-Butyl (3R)-3-benzyl-4-{[1-(2-{[(ethyl)(methyl)amino]methyl}-2-hydroxycyclohexyl)-5-phenyl-1H-imidazol-4-yl]carbonyl}piperazine-1-carboxylate

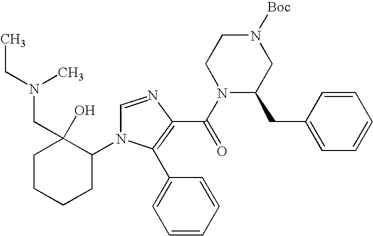

MS (ESI+, m/e) 616 (M+1)

### Reference Example 622

### tert-Butyl (3R)-3-benzyl-4-{[1-(2-{[(2-furylmethyl)amino]methyl}-2-hydroxycyclohexyl)-5-phenyl-1H-imidazol-4-yl]carbonyl}piperazine-1-carboxylate

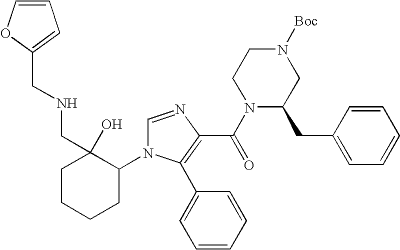

MS (ESI+, m/e) 654 (M+1)

### Reference Example 623

### tert-Butyl (3R)-4-{[1-(2-{[(acetyl)(ethyl)amino]methyl}-2-hydroxycyclohexyl)-5-phenyl-1H-imidazol-4-yl]carbonyl}-3-benzylpiperazine-1-carboxylate

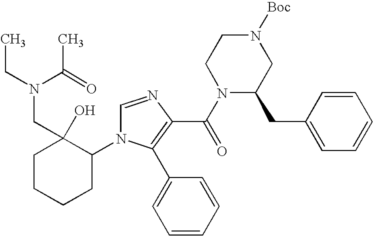

tert-Butyl (3R)-3-benzyl-4-[(1-{2-[(ethylamino)methyl]-2-hydroxycyclohexyl}-5-phenyl-1H-imidazol-4-yl)carbonyl]piperazine-1-carboxylate (120 mg) was dissolved in pyridine (2 ml), and the solution was ice-cooled. Acetic anhydride (19 μl) was added, and the mixture was stirred at room temperature for 15 hr. To the reaction mixture was added aqueous sodium bicarbonate, and the mixture was extracted with ethyl acetate. The extract was washed with saturated brine, dried over anhydrous magnesium sulfate, and concentrated under reduced pressure. The residue was subjected to basic silica gel column chromatography, and the fraction eluted with ethyl acetate-methanol (9:1) was concentrated under reduced pressure to give the object compound (105 mg) as an amorphous solid.

MS (ESI+, m/e) 644 (M+1)

### Reference Example 624

### tert-Butyl (3R)-3-benzyl-4-{([1-(2-ethyl-2-hydroxycyclohexyl)-5-phenyl-1H-imidazol-4-yl]carbonyl}piperazine-1-carboxylate

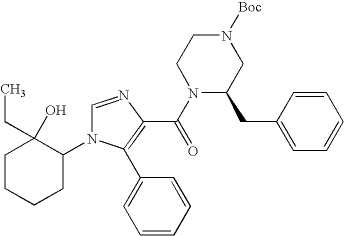

Copper iodide (160 mg) was suspended in THF (5 ml), and the suspension was ice-cooled. Methylmagnesium bromide (1M THF solution, 1.6 ml) was added, and the mixture was stirred at 0° C. for 30 min. A solution of tert-butyl (3R)-3-benzyl-4-{[1-(1-oxaspiro[2.5]oct-4-yl)-5-phenyl-1H-imidazol-4-yl]carbonyl}piperazine-1-carboxylate (111 mg) in THF (5 ml) was added thereto, and the mixture was stirred at room temperature for 3 hr. To the reaction mixture was added saturated aqueous ammonium chloride solution, and the mixture was extracted with ethyl acetate. The extract was washed successively with saturated aqueous sodium hydrogen carbonate and saturated brine, dried over anhydrous magnesium sulfate, and concentrated under reduced pressure. The residue was subjected to silica gel column chromatography, and the fraction eluted with ethyl acetate-methanol (4:1) was concentrated under reduced pressure to give the object compound (73 mg) as an amorphous solid.

MS (ESI+, m/e) 573 (M+1)

In the same manner as in Reference Example 624, the following compound (Reference Example 625) was obtained.

### Reference Example 625

### tert-Butyl (3R)-3-benzyl-4-{[1-(2-hydroxy-2-propylcyclohexyl)-5-phenyl-1H-imidazol-4-yl]carbonyl}piperazine-1-carboxylate

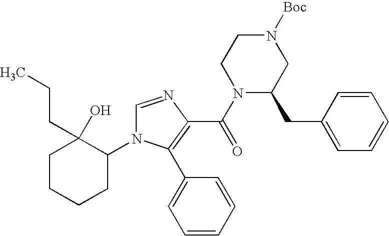

MS (ESI+, m/e) 587 (M+1)

### Reference Example 626

### tert-Butyl (3R)-3-benzyl-4-({1-[(1R,2R)-2-(cyclopropylmethyl)-2-hydroxycyclohexyl]-5-phenyl-1H-imidazol-4-yl}carbonyl)piperazine-1-carboxylate and tert-butyl (3R)-3-benzyl-4-({1-[(1S,2S)-2-(cyclopropylmethyl)-2-hydroxycyclohexyl]-5-phenyl-1H-imidazol-4-yl}carbonyl)piperazine-1-carboxylate

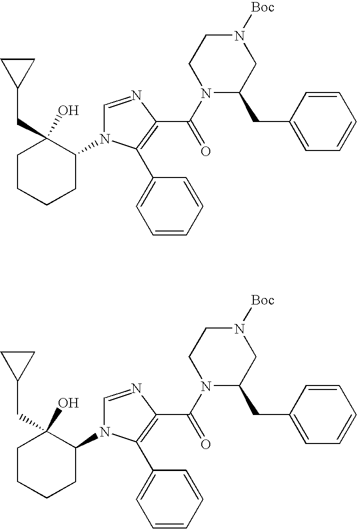

Copper iodide (144 mg) was suspended in THF (5 ml), and the suspension was ice-cooled. Cyclopropylmagnesium bromide (0.5M THF solution, 2.9 ml) was added, and the mixture was stirred at 0° C. for 30 min. A solution of tert-butyl (3R)-3-benzyl-4-{[1-(1-oxaspiro[2.5]oct-4-yl)-5-phenyl-1H-imidazol-4-yl]carbonyl}piperazine-1-carboxylate (200 mg) in THF (5 ml) was added thereto, and the mixture was stirred at room temperature for 2 hr. To the reaction mixture was added saturated aqueous ammonium chloride solution, and the mixture was extracted with ethyl acetate. The extract was washed with saturated brine, dried over anhydrous magnesium sulfate, and concentrated under reduced pressure. The residue was subjected to silica gel column chromatography, and the fractions eluted with ethyl acetate-methanol (4:1) were concentrated under reduced pressure to give tert-butyl (3R)-3-benzyl-4-({1-[(1R,2R)-2-(cyclopropylmethyl)-2-hydroxycyclohexyl]-5-phenyl-1H-imidazol-4-yl}carbonyl)piperazine-1-carboxylate (49 mg) as an amorphous solid, and tert-butyl (3R)-3-benzyl-4-({1-[(1S,2S)-2-(cyclopropylmethyl)-2-hydroxycyclohexyl]-5-phenyl-1H-imidazol-4-yl}carbonyl)piperazine-1-carboxylate (214 mg) as an amorphous solid.

MS (ESI+, m/e) 599 (M+1)

MS (ESI+, m/e) 599 (M+1)

In the same manner as in Reference Example 626, the following compound (Reference Example 627) was obtained.

### Reference Example 627

### tert-Butyl (3R)-3-benzyl-4-({1-[(1R,2S)-2-butyl-2-hydroxycyclohexyl]-5-phenyl-1H-imidazol-4-yl}carbonyl)piperazine-1-carboxylate and tert-butyl (3R)-3-benzyl-4-({1-[(1S,2R)-2-butyl-2-hydroxycyclohexyl]-5-phenyl-1H-imidazol-4-yl}carbonyl)piperazine-1-carboxylate

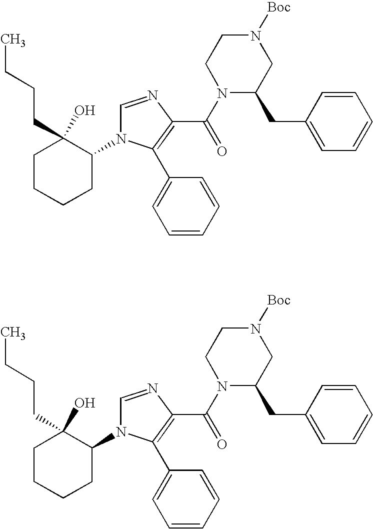

MS (ESI+, m/e) 601 (M+1)

MS (ESI+, m/e) 601 (M+1)

### Reference Example 628

### tert-Butyl (3R)-3-benzyl-4-{[1-(2-hydroxy-2-{[2-(methylsulfonyl)ethoxy]methyl}cyclohexyl)-5-phenyl-1H-imidazol-4-yl]carbonyl}piperazine-1-carboxylate

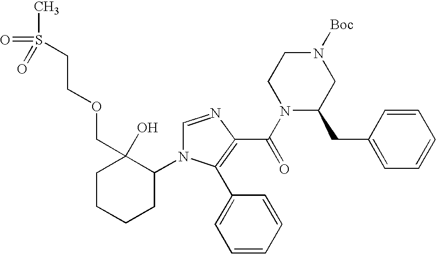

Sodium hydride (60% in oil) (100 mg) was suspended in DMF (3 ml), 2-(methylthio)ethanol (280 mg) was added, and the mixture was stirred at room temperature for 30 min. tert-Butyl (3R)-3-benzyl-4-{[1-(1-oxaspiro[2.5]oct-4-yl)-5-phenyl-1H-imidazol-4-yl]carbonyl}piperazine-1-carboxylate (280 mg) was added thereto, and the mixture was stirred at 60° C. for 15 hr. To the reaction mixture was added aqueous sodium bicarbonate, and the mixture was extracted with ethyl acetate. The extract was washed with saturated brine, dried over anhydrous magnesium sulfate, and concentrated under reduced pressure. The residue was subjected to silica gel column chromatography, and the fraction eluted with ethyl acetate-methanol (9:1) was concentrated under reduced pressure to give tert-butyl (3R)-3-benzyl-4-{[1-(2-hydroxy-2-{[2-(methylthio)ethoxy]methyl}cyclohexyl)-5-phenyl-1H-imidazol-4-yl]carbonyl}piperazine-1-carboxylate (270 mg) as an amorphous solid. 145 mg therefrom was dissolved in dichloromethane (3 ml), and the solution was ice-cooled. mCPBA (119 mg) was added, and the mixture was stirred at 0° C. for 30 min. To the reaction mixture was added aqueous sodium thiosulfate solution, and the mixture was extracted with ethyl acetate. The extract was washed successively with saturated aqueous sodium hydrogen carbonate and saturated brine, and dried over anhydrous sodium sulfate, and the solvent was evaporated under reduced pressure. The residue was subjected to silica gel column chromatography, and the fraction eluted with ethyl acetate-methanol (7:3) was concentrated under reduced pressure to give the object compound (119 mg) as an amorphous solid.

MS (ESI+, m/e) 681 (M+1)

In the same manner as in Reference Example 628, the following compounds (Reference Examples 629-631) were obtained.

### Reference Example 629

### tert-Butyl (3R)-3-benzyl-4-{[1-(2-hydroxy-2-{[3-(methylsulfonyl)propoxy]methyl}cyclohexyl)-5-phenyl-1H-imidazol-4-yl]carbonyl}piperazine-1-carboxylate

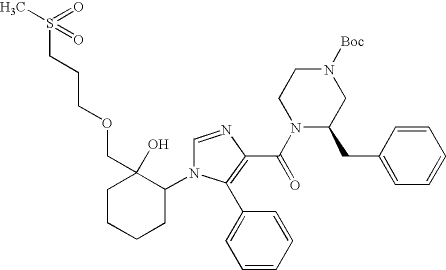

MS (ESI+, m/e) 695 (M+1)

### Reference Example 630

### tert-Butyl (3R)-3-benzyl-4-{[1-(2-{[(1,1-dioxidotetrahydro-2H-thiopyran-4-yl)oxy]methyl}-2-hydroxycyclohexyl)-5-phenyl-1H-imidazol-4-yl]carbonyl}piperazine-1-carboxylate

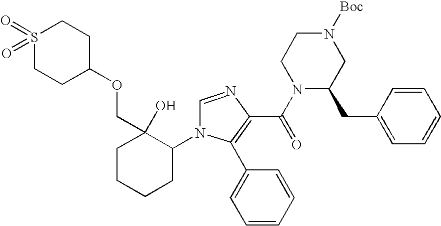

MS (ESI+, m/e) 707 (M+1)

### Reference Example 631

### tert-Butyl (3R)-3-benzyl-4-{[1-(2-{[(1,1-dioxidotetrahydro-2H-thiopyran-4-yl)methoxy]methyl}-2-hydroxycyclohexyl)-5-phenyl-1H-imidazol-4-yl]carbonyl}piperazine-1-carboxylate

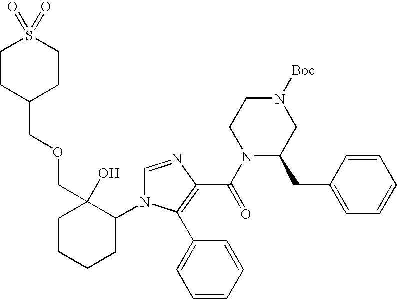

MS (ESI+, m/e) 721 (M+1)

### Reference Example 632

### tert-Butyl (3R)-3-benzyl-4-[(1-{(1S,2R)-2-hydroxy-2-[(1,3-thiazol-2-ylmethoxy)methyl]cyclohexyl}-5-phenyl-1H-imidazol-4-yl)carbonyl]piperazine-1-carboxylate

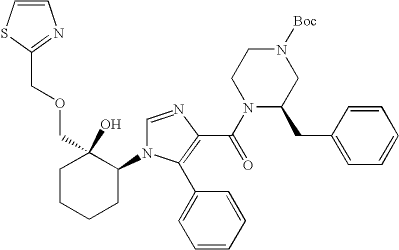

Sodium hydride (60% in oil) (24 mg) was suspended in DMF (3 ml), 1,3-thiazol-2-ylmethanol (81 mg) was added, and the mixture was stirred at room temperature for 30 min. tert-Butyl (3R)-3-benzyl-4-({1-[(1S,2R)-2-(chloromethyl)-2-hydroxycyclohexyl]-5-phenyl-1H-imidazol-4-yl}carbonyl)piperazine-1-carboxylate (119 mg) was added thereto, and the mixture was stirred at 60° C. for 15 hr. To the reaction mixture was added aqueous sodium bicarbonate, and the mixture was extracted with ethyl acetate. The extract was washed with saturated brine, dried over anhydrous magnesium sulfate, and concentrated under reduced pressure. The residue was subjected to silica gel column chromatography, and the fraction eluted with ethyl acetate-methanol (4:1) was concentrated under reduced pressure to give the object compound (96 mg) as an amorphous solid.

MS (ESI+, m/e) 672 (M+1)

In the same manner as in Reference Example 632, the following compounds (Reference Examples 633-637) were obtained.

### Reference Example 633

### tert-Butyl (3R)-3-benzyl-4-{[1-((1S,2R)-2-hydroxy-2-{[2-(2-hydroxyethoxy)ethoxy]methyl}cyclohexyl)-5-phenyl-1H-imidazol-4-yl]carbonyl}piperazine-1-carboxylate

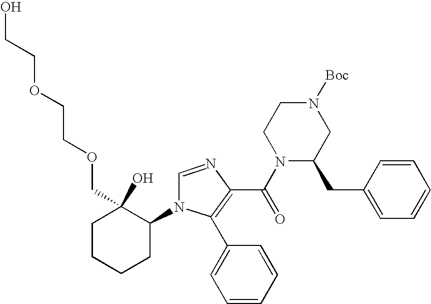

MS (ESI+, m/e) 663 (M+1)

### Reference Example 634

### tert-Butyl (3R)-3-benzyl-4-{[1-((1S,2R)-2-{[3-(dimethylamino)propoxy]methyl}-2-hydroxycyclohexyl)-5-phenyl-1H-imidazol-4-yl]carbonyl}piperazine-1-carboxylate

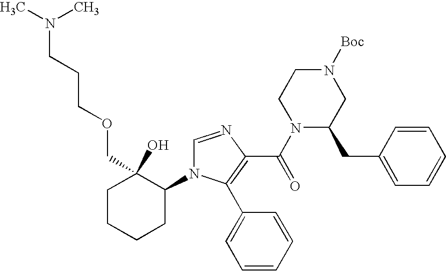

MS (ESI+, m/e) 660 (M+1)

### Reference Example 635

### tert-Butyl (3R)-3-benzyl-4-[(1-{(1S,2R)-2-hydroxy-2-[(pyridin-2-ylmethoxy)methyl]cyclohexyl}-5-phenyl-1H-imidazol-4-yl)carbonyl]piperazine-1-carboxylate

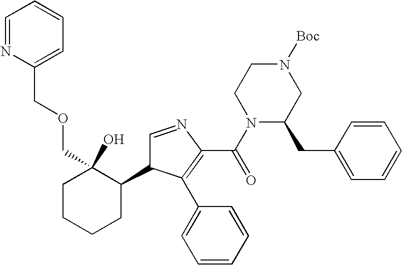

MS (ESI+, m/e) 666 (M+1)

### Reference Example 636

### tert-Butyl (3R)-4-[(1-{(1S,2R)-2-[(1H-benzimidazol-2-ylmethoxy)methyl]-2-hydroxycyclohexyl}-5-phenyl-1H-imidazol-4-yl)carbonyl]-3-benzylpiperazine-1-carboxylate

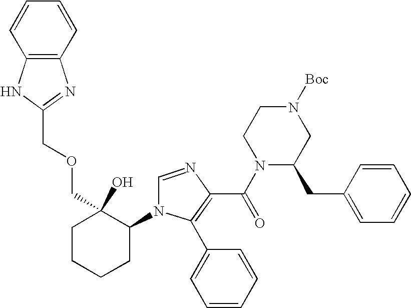

MS (ESI+, m/e) 705 (M+1)

### Reference Example 637

### tert-Butyl (3R)-3-benzyl-4-[(1-{(1S,2R)-2-[(2,3-dihydro-1H-inden-2-yloxy)methyl]-2-hydroxycyclohexyl}-5-phenyl-1H-imidazol-4-yl)carbonyl]piperazine-1-carboxylate

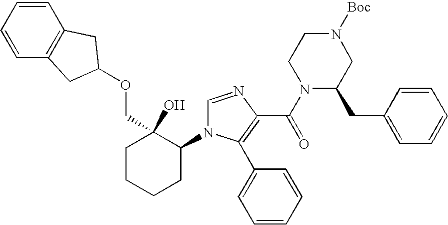

MS (ESI+, m/e) 691 (M+1)

### Reference Example 638

### tert-Butyl (3R)-3-benzyl-4-({1-[2-(2-cyanoethyl)-2-hydroxycyclohexyl]-5-phenyl-1H-imidazol-4-yl}carbonyl)piperazine-1-carboxylate

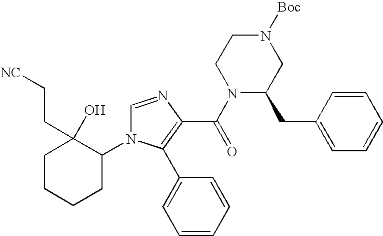

Lithium bis(trimethylsilyl)amide (1.1M THF solution, 3.6 ml) was dissolved in THF (5 ml), and the solution was cooled to −10° C. A solution of acetonitrile (221 μl) in THF (3 ml) was added over 3 min, and the mixture was stirred at −10° C. for 30 min. A solution of tert-butyl (3R)-3-benzyl-4-{[1-(1-oxaspiro[2.5]oct-4-yl)-5-phenyl-1H-imidazol-4-yl]carbonyl}piperazine-1-carboxylate (557 mg) in THF (5 ml) was added thereto, and the mixture was stirred at −10° C. for 1 hr, and then at room temperature for 3 hr. To the reaction mixture was added saturated aqueous ammonium chloride solution, and the mixture was extracted with ethyl acetate. The extract was washed with saturated brine, dried over anhydrous magnesium sulfate, and concentrated under reduced pressure. The residue was subjected to silica gel column chromatography, and the fraction eluted with ethyl acetate-methanol (9:1) was concentrated under reduced pressure to give the object compound (370 mg) as an oil.

MS (ESI+, m/e) 598 (M+1)

### Reference Example 639

### tert-Butyl (3R)-3-benzyl-4-[(1-{2-[(ethylthio)methyl]-2-hydroxycyclohexyl}-5-phenyl-1H-imidazol-4-yl)carbonyl]piperazine-1-carboxylate

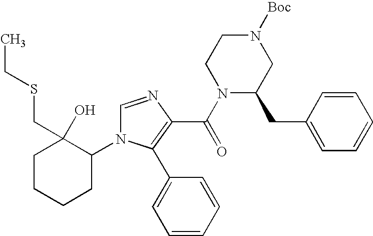

tert-Butyl (3R)-3-benzyl-4-{[1-(1-oxaspiro[2.5]oct-4-yl)-5-phenyl-1H-imidazol-4-yl]carbonyl}piperazine-1-carboxylate (334 mg) was dissolved in DMF (5 ml), sodium ethanethiolate (80%) (315 mg) was added, and the mixture was stirred at 60° C. for 15 hr. To the reaction mixture was added aqueous sodium bicarbonate, and the mixture was extracted with ethyl acetate. The extract was washed with saturated brine, dried over anhydrous magnesium sulfate, and concentrated under reduced pressure. The residue was subjected to silica gel column chromatography, and the fraction eluted with ethyl acetate was concentrated under reduced pressure to give the object compound (324 mg) as an amorphous solid.

MS (ESI+, m/e) 619 (M+1)

### Reference Example 640

### tert-Butyl (3R)-3-benzyl-4-[(1-{2-[(ethylsulfonyl)methyl]-2-hydroxycyclohexyl}-5-phenyl-1H-imidazol-4-yl)carbonyl]piperazine-1-carboxylate

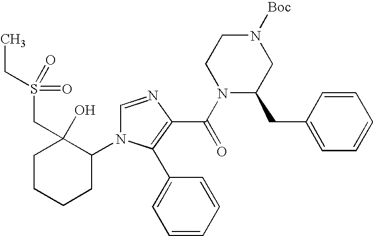

tert-Butyl (3R)-3-benzyl-4-[(1-{2-[(ethylthio)methyl]-2-hydroxycyclohexyl}-5-phenyl-1H-imidazol-4-yl)carbonyl]piperazine-1-carboxylate (105 mg) was dissolved in dichloromethane (5 ml), and the solution was ice-cooled, m-chloroperbenzoic acid (95 mg) was added, and the mixture was stirred at 0° C. for 30 min. To the reaction mixture was added aqueous sodium thiosulfate solution, and the mixture was extracted with ethyl acetate. The extract was washed successively with saturated aqueous sodium hydrogen carbonate and saturated brine, and dried over anhydrous sodium sulfate, and the solvent was evaporated under reduced pressure. The residue was subjected to silica gel column chromatography, and the fraction eluted with ethyl acetate-methanol (7:3) was concentrated under reduced pressure to give the object compound (52 mg) as an amorphous solid.

MS (ESI+, m/e) 651 (M+1)

### Reference Example 641

### tert-Butyl (3R)-3-benzyl-4-{[1-(2-hydroxy-2-{[(3-methyloxetan-3-yl)methoxy]methyl}cyclohexyl)-5-phenyl-1H-imidazol-4-yl]carbonyl}piperazine-1-carboxylate

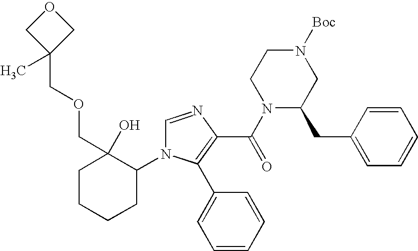

Sodium hydride (60% in oil) (40 mg) was suspended in DMF (3 ml), (3-methyloxetan-3-yl)methanol (120 mg) was added, and the mixture was stirred at room temperature for 30 min. tert-Butyl (3R)-3-benzyl-4-{[1-(1-oxaspiro[2.5]oct-4-yl)-5-phenyl-1H-imidazol-4-yl]carbonyl}piperazine-1-carboxylate (110 mg) was added thereto, and the mixture was stirred at 60° C. for 15 hr. To the reaction mixture was added aqueous sodium bicarbonate, and the mixture was extracted with ethyl acetate. The extract was washed with saturated brine, dried over anhydrous magnesium sulfate, and concentrated under reduced pressure. The residue was subjected to silica gel column chromatography, and the fraction eluted with ethyl acetate-methanol (9:1) was concentrated under reduced pressure to give the object compound (100 mg) as an amorphous solid.

MS (ESI+, m/e) 659 (M+1)

### Reference Example 642

### tert-Butyl (3R)-3-benzyl-4-{[5-(3-fluorophenyl)-1-(cis-1-oxaspiro[2.5]oct-4-yl)-1H-imidazol-4-yl]carbonyl}piperazine-1-carboxylate

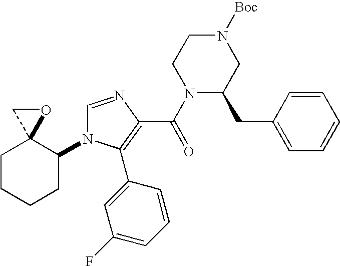

tert-Butyl (3R)-3-benzyl-4-({5-(3-fluorophenyl)-1-[(1S,2S)-2-hydroxycyclohexyl]-1H-imidazol-4-yl}carbonyl)piperazine-1-carboxylate (825 mg) was dissolved in 1,2-dichloroethane (30 ml), Dess-Martin reagent (929 mg) was added thereto, and the mixture was stirred at room temperature for 3 hr. The reaction mixture was poured into water, and the mixture was extracted with chloroform. The extract was concentrated under reduced pressure, and the residue was dissolved in ethyl acetate-THF. 10% Aqueous sodium thiosulfate solution was added thereto, and the mixture was stirred for 30 min. The organic layer was washed successively with saturated aqueous sodium hydrogen carbonate and saturated brine, and dried over anhydrous sodium sulfate, and the solvent was evaporated under reduced pressure. To the residue was added ethyl acetate, and the precipitated crystals were collected by filtration to give tert-butyl (3R)-3-benzyl-4-{[5-(3-fluorophenyl)-1-(2-oxocyclohexyl)-1H-imidazol-4-yl]carbonyl}piperazine-1-carboxylate (650 mg).

Trimethylsulfoxonium iodide (376 mg) was dissolved in DMSO (10 ml), sodium hydride (60% in oil, 55 mg) was added thereto, and the mixture was stirred at room temperature for 30 min. A solution of the oxo form obtained in the above in DMSO (20 ml) was added thereto, and the mixture was stirred at room temperature for 30 min. The reaction mixture was poured into saturated aqueous ammonium chloride solution, and the mixture was extracted with ethyl acetate. The extract was dried over anhydrous sodium sulfate, and the solvent was evaporated under reduced pressure. The residue was subjected to silica gel column chromatography, and the fraction eluted with ethyl acetate was concentrated under reduced pressure to give the object compound (401 mg) as an amorphous solid.

MS (ESI+, m/e) 575 (M+1)

### Reference Example 643

### tert-Butyl (3R)-3-benzyl-4-({5-(3-fluorophenyl)-1-[cis-2-hydroxy-2-(methoxymethyl)cyclohexyl]-1H-imidazol-4-yl}carbonyl)piperazine-1-carboxylate

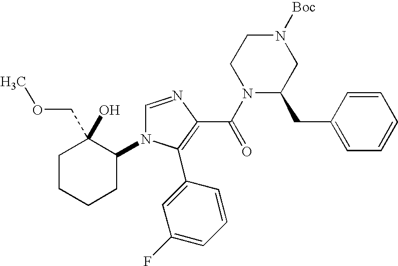

tert-Butyl (3R)-3-benzyl-4-{[5-(3-fluorophenyl)-1-(cis-1-oxaspiro[2.5]oct-4-yl)-1H-imidazol-4-yl]carbonyl}piperazine-1-carboxylate (390 mg) was dissolved in methanol (5 ml), sodium methoxide (28% methanol solution, 650 μl) was added, and the mixture was stirred at 50° C. for 15 hr. The reaction mixture was concentrated under reduced pressure, and the residue was partitioned between ethyl acetate and water. The organic layer was washed with saturated brine, and dried over anhydrous sodium sulfate, and the solvent was evaporated under reduced pressure. The residue was subjected to silica gel column chromatography, and the fraction eluted with ethyl acetate was concentrated under reduced pressure to give the object compound (337 mg) as an amorphous solid.

MS (ESI+, m/e) 607 (M+1)

### Reference Example 644

### tert-Butyl (3R)-3-(2-hydroxyethyl)-4-({1-[(1R,2S)-2-hydroxy-2-(methoxymethyl)cyclohexyl]-5-phenyl-1H-imidazol-4-yl}carbonyl)piperazine-1-carboxylate

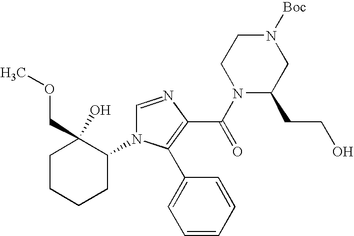

(1S,2R)-2-(4-{[(2R)-4-Benzyl-2-(2-hydroxyethyl)piperazin-1-yl]carbonyl}-5-phenyl-1H-imidazol-1-yl)-1-(methoxymethyl)cyclohexanol (3.39 g) was dissolved in methanol (200 ml), 20% palladium hydroxide-carbon (50% containing water, 500 mg) was added thereto, and the mixture was subjected to catalytic reduction at ambient temperature and normal pressure for 12 hr. The catalyst was filtered off, and the filtrate was concentrated under reduced pressure. The residue was dissolved in THF (50 ml), and the solution was ice-cooled. Di-tert-butyl bicarbonate (1.66 g) was added, and the mixture was stirred at room temperature for 3 hr. The solvent was evaporated under reduced pressure. The residue was subjected to basic silica gel column chromatography, and the fraction eluted with ethyl acetate-methanol (9:1) was concentrated under reduced pressure to give the object compound (3.52 g) as an amorphous solid.

MS (ESI+, m/e) 543 (M+1)

### Reference Example 645

### tert-Butyl (3R)-4-({1-[(1R,2S)-2-hydroxy-2-(methoxymethyl)cyclohexyl]-5-phenyl-1H-imidazol-4-yl}carbonyl)-3-{2-[(pyrrolidin-1-ylcarbonyl)oxy]ethyl}piperazine-1-carboxylate

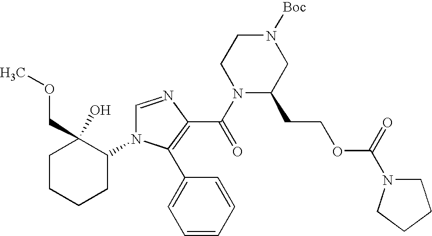

tert-Butyl (3R)-3-(2-hydroxyethyl)-4-({1-[(1R,2S)-2-hydroxy-2-(methoxymethyl)cyclohexyl]-5-phenyl-1H-imidazol-4-yl}carbonyl)piperazine-1-carboxylate (108 mg) and DMAP (73 mg) were dissolved in THF (5 ml), 4-nitrophenyl chloroformate (60 mg) was added, and the mixture was stirred at room temperature for 1 hr. To the reaction mixture was added pyrrolidine (142 mg), and the mixture was further stirred at room temperature for 2 hr. To the reaction mixture was added saturated aqueous sodium hydrogen carbonate, and the mixture was extracted with ethyl acetate. The extract was washed with saturated brine, dried over anhydrous magnesium sulfate, and concentrated under reduced pressure. The residue was subjected to basic silica gel column chromatography, and the fraction eluted with ethyl acetate-methanol (9:1) was concentrated under reduced pressure to give the object compound (90 mg) as an amorphous solid.

MS (ESI+, m/e) 640 (M+1)

### Reference Example 646

### tert-Butyl (3R)-4-({1-[(1R,2S)-2-hydroxy-2-(methoxymethyl)cyclohexyl]-5-phenyl-1H-imidazol-4-yl}carbonyl)-3-(2-oxoethyl)piperazine-1-carboxylate

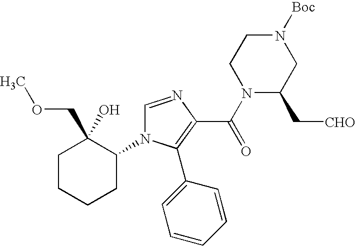

tert-Butyl (3R)-3-(2-hydroxyethyl)-4-({1-[(1R,2S)-2-hydroxy-2-(methoxymethyl)cyclohexyl]-5-phenyl-1H-imidazol-4-yl}carbonyl)piperazine-1-carboxylate (560 mg) was dissolved in 1,2-dichloroethane (10 ml), Dess-Martin reagent (657 mg) was added thereto, and the mixture was stirred at room temperature for 2 hr. The reaction mixture was poured into water, and the mixture was extracted with chloroform. The extract was concentrated under reduced pressure, and the residue was dissolved in ethyl acetate-THF. 10% Aqueous sodium thiosulfate solution was added thereto, and the mixture was stirred at room temperature for 30 min. The organic layer was washed successively with saturated aqueous sodium hydrogen carbonate and saturated brine, and dried over anhydrous sodium sulfate, and the solvent was evaporated under reduced pressure. The residue was subjected to silica gel column chromatography, and the fraction eluted with ethyl acetate-methanol (17:3) was concentrated under reduced pressure to give the object compound (411 mg) as an amorphous solid.

MS (ESI+, m/e) 541 (M+1-“Boc”).

### Reference Example 647

### tert-Butyl (3R)-4-({1-[((1R,2S)-2-hydroxy-2-(methoxymethyl)cyclohexyl]-5-phenyl-1H-imidazol-4-yl}carbonyl)-3-{(2R)-2-hydroxy-2-[6-(trifluoromethyl)pyridin-2-yl]ethyl}piperazine-1-carboxylate and tert-butyl (3R)-4-({1-[(1R,2S)-2-hydroxy-2-(methoxymethyl)cyclohexyl]-5-phenyl-1H-imidazol-4-yl}carbonyl)-3-{(2S)-2-hydroxy-2-[6-(trifluoromethyl)pyridin-2-yl]ethyl}piperazine-1-carboxylate

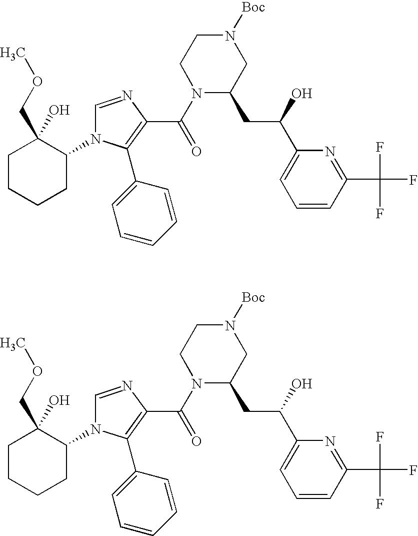

2-Bromo-6-(trifluoromethyl)pyridine (375 mg) was dissolved in diethyl ether (10 ml), and the solution was cooled to −78° C. Butyllithium (1.6M hexane solution, 0.95 ml) was added, and the mixture was stirred at the same temperature for 30 min. To the reaction mixture was added a solution of tert-butyl (3R)-4-({1-[(1R,2S)-2-hydroxy-2-(methoxymethyl)cyclohexyl]-5-phenyl-1H-imidazol-4-yl}carbonyl)-3-(2-oxoethyl)piperazine-1-carboxylate (250 mg) in diethyl ether (10 ml) at −78° C., and the mixture was stirred at the same temperature for 3 hr. To the reaction mixture was added saturated aqueous ammonium chloride solution, and the mixture was extracted with ethyl acetate. The extract was washed with saturated brine, and dried over anhydrous sodium sulfate, and the solvent was evaporated under reduced pressure. The residue was subjected to basic silica gel column chromatography, and the fractions eluted with ethyl acetate-methanol (19:1) were concentrated under reduced pressure to give tert-butyl (3R)-4-({1-[(1R,2S)-2-hydroxy-2-(methoxymethyl)cyclohexyl]-5-phenyl-1H-imidazol-4-yl}carbonyl)-3-{(2R)-2-hydroxy-2-[6-(trifluoromethyl)pyridin-2-yl]ethyl}piperazine-1-carboxylate (65 mg) as an amorphous solid, and tert-butyl (3R)-4-({1-[(1R,2S)-2-hydroxy-2-(methoxymethyl)cyclohexyl]-5-phenyl-1H-imidazol-4-yl}carbonyl)-3-{(2S)-2-hydroxy-2-[6-(trifluoromethyl)pyridin-2-yl]ethyl}piperazine-1-carboxylate (73 mg) as an amorphous solid.

MS (ESI+, m/e) 688 (M+1)

MS (ESI+, m/e) 688 (M+1)

### Reference Example 648

### tert-Butyl (3R)-4-({1-[(1R,2S)-2-hydroxy-2-(methoxymethyl)cyclohexyl]-5-phenyl-1H-imidazol-4-yl}carbonyl)-3-[(2R)-2-hydroxy-2-phenylethyl]piperazine-1-carboxylate and tert-butyl (3R)-4-({1-[(1R,2S)-2-hydroxy-2-(methoxymethyl)cyclohexyl]-5-phenyl-1H-imidazol-4-yl}carbonyl)-3-[(2S)-2-hydroxy-2-phenylethyl]piperazine-1-carboxylate

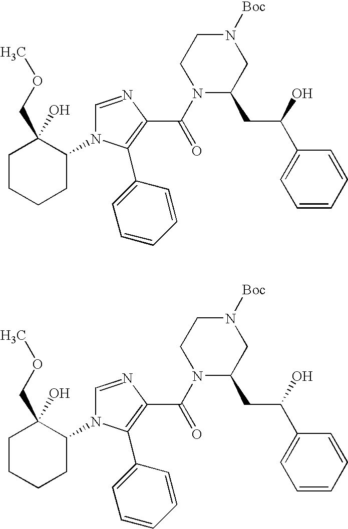

tert-Butyl (3R)-4-({1-[(1R,2S)-2-hydroxy-2-(methoxymethyl)cyclohexyl]-5-phenyl-1H-imidazol-4-yl}carbonyl)-3-(2-oxoethyl)piperazine-1-carboxylate (150 mg) was dissolved in THF (10 ml), and the solution was ice-cooled. Phenylmagnesium bromide (1.08M THF solution, 0.85 ml) was added, and the mixture was stirred at 0° C. for 1 hr. To the reaction mixture was added saturated aqueous ammonium chloride solution, and the mixture was extracted with ethyl acetate. The extract was washed with saturated brine, and dried over anhydrous sodium sulfate, and the solvent was evaporated under reduced pressure. The residue was subjected to basic silica gel column chromatography, and the fractions eluted with ethyl acetate-methanol (19:1) were concentrated under reduced pressure to give tert-butyl (3R)-4-({1-[(1R,2S)-2-hydroxy-2-(methoxymethyl)cyclohexyl]-5-phenyl-1H-imidazol-4-yl}carbonyl)-3-[(2R)-2-hydroxy-2-phenylethyl]piperazine-1-carboxylate (45 mg) as an amorphous solid, and tert-butyl (3R)-4-({1-[(1R,2S)-2-hydroxy-2-(methoxymethyl)cyclohexyl]-5-phenyl-1H-imidazol-4-yl}carbonyl)-3-[(2S)-2-hydroxy-2-phenylethyl]piperazine-1-carboxylate (73 mg) as an amorphous solid.

MS (ESI+, m/e) 619 (M+1)

MS (ESI+, m/e) 619 (M+1)

### Reference Example 649

### (1S,2R)-2-(4-{[(2R)-4-Benzyl-2-(2-phenoxyethyl)piperazin-1-yl]carbonyl}-5-phenyl-1H-imidazol-1-yl)-1-(methoxymethyl)cyclohexanol

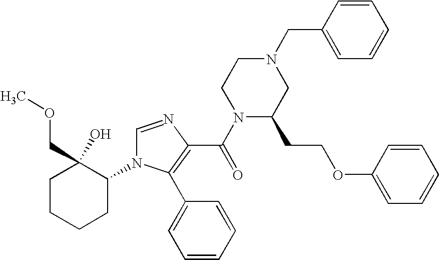

(1S,2R)-2-(4-{[(2R)-4-Benzyl-2-(2-hydroxyethyl)piperazin-1-yl]carbonyl}-5-phenyl-1H-imidazol-1-yl)-1-(methoxymethyl)cyclohexanol (242 mg), phenol (64 mg) and triphenylphosphine (239 mg) were dissolved in THF (15 ml), DEAD (40% toluene solution, 396 μl) was added, and the mixture was stirred at room temperature for 5 hr. The solvent was evaporated under reduced pressure, and the residue was dissolved in ethyl acetate (20 ml). The mixture was washed successively with saturated aqueous sodium hydrogen carbonate and saturated brine, and dried over anhydrous magnesium sulfate, and the solvent was evaporated under reduced pressure. The residue was subjected to basic silica gel column chromatography, and the object fraction was concentrated under reduced pressure to give the object compound (32 mg) as an amorphous solid.

MS (ESI+, m/e) 609 (M+1)

### Reference Example 650

### tert-Butyl (3R)-4-({1-[(1R,2S)-2-hydroxy-2-(methoxymethyl)cyclohexyl]-5-phenyl-1H-imidazol-4-yl}carbonyl)-3-[2-(pyridin-2-yloxy)ethyl]piperazine-1-carboxylate

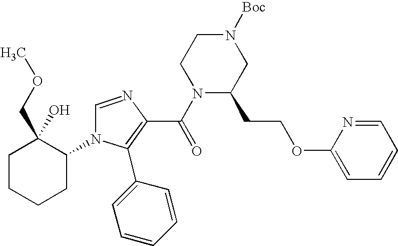

tert-Butyl (3R)-3-(2-hydroxyethyl)-4-({1-[(1R,2S)-2-hydroxy-2-(methoxymethyl)cyclohexyl]-5-phenyl-1H-imidazol-4-yl}carbonyl)piperazine-1-carboxylate (109 mg) was dissolved in DMF (3 ml), and the solution was ice-cooled. Sodium hydride (60% in oil, 18 mg) was added thereto, and the mixture was stirred at 0° C. for 10 min. After stirring, 2-bromopyridine (29 μl) was added thereto at 0° C., and the mixture was stirred at room temperature for 15 hr. The reaction mixture was partitioned between ethyl acetate and water. The organic layer was washed with saturated brine, and dried over anhydrous sodium sulfate, and the solvent was evaporated under reduced pressure. The residue was subjected to silica gel column chromatography, and the fraction eluted with ethyl acetate-methanol (19:1) was concentrated under reduced pressure to give the object compound (16 mg) as an oil.

MS (ESI+, m/e) 620 (M+1)

In the same manner as in Reference Example 650, the following compounds (Reference Examples 651-656) were obtained.

### Reference Example 651

### tert-Butyl (3R)-4-({1-[(1R,2S)-2-hydroxy-2-(methoxymethyl)cyclohexyl]-5-phenyl-1H-imidazol-4-yl}carbonyl)-3-(2-{[6-(trifluoromethyl)pyridin-2-yl]oxy}ethyl)piperazine-1-carboxylate

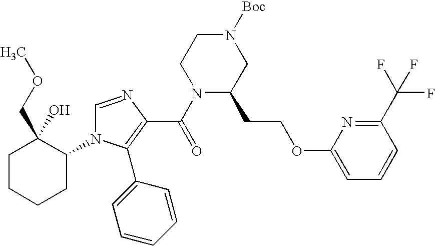

MS (ESI+, m/e) 688 (M+1)

### Reference Example 652

### tert-Butyl (3R)-4-({1-[(1R,2S)-2-hydroxy-2-(methoxymethyl)cyclohexyl]-5-phenyl-1H-imidazol-4-yl}carbonyl)-3-[2-(pyrimidin-2-yloxy)ethyl]piperazine-1-carboxylate

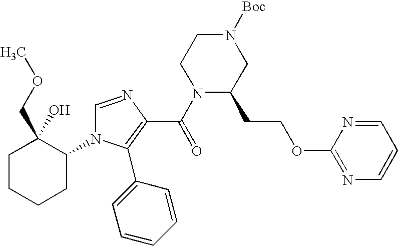

MS (ESI+, m/e) 621 (M+1)

### Reference Example 653

### tert-Butyl (3R)-4-({1-[(1R,2S)-2-hydroxy-2-(methoxymethyl)cyclohexyl]-5-phenyl-1H-imidazol-4-yl}carbonyl)-3-(2-{[4-(trifluoromethyl)pyridin-2-yl]oxy}ethyl)piperazine-1-carboxylate

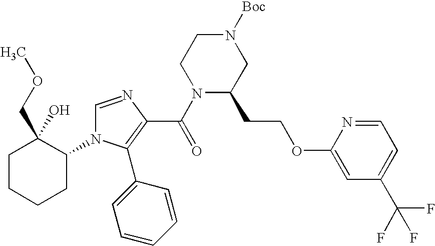

MS (ESI+, m/e) 688 (M+1)

### Reference Example 654

### tert-Butyl (3R)-4-({1-[(1R,2S)-2-hydroxy-2-(methoxymethyl)cyclohexyl]-5-phenyl-1H-imidazol-4-yl}carbonyl)-3-(2-{[3-(trifluoromethyl)pyridin-2-yl]oxy}ethyl)piperazine-1-carboxylate

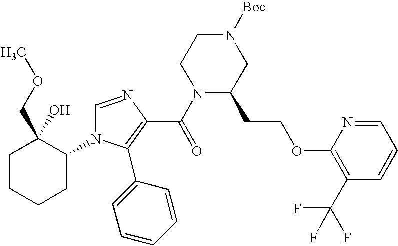

MS (ESI+, m/e) 688 (M+1)

### Reference Example 655

### tert-Butyl (3R)-3-{2-[(5-cyanopyridin-2-yl)oxy]ethyl}-4-({1-[(1R,2S)-2-hydroxy-2-(methoxymethyl)cyclohexyl]-5-phenyl-1H-imidazol-4-yl}carbonyl)piperazine-1-carboxylate

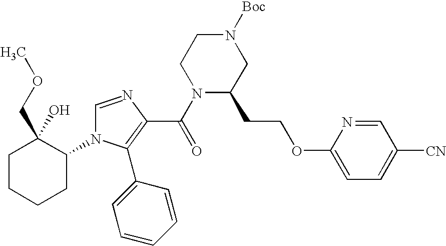

MS (ESI+, m/e) 645 (M+1)

### Reference Example 656

### tert-Butyl (3R)-4-({1-[(1R,2S)-2-hydroxy-2-(methoxymethyl)cyclohexyl]-5-phenyl-1H-imidazol-4-yl}carbonyl)-3-(2-{[5-(trifluoromethyl)pyridin-2-yl]oxy}ethyl)piperazine-1-carboxylate

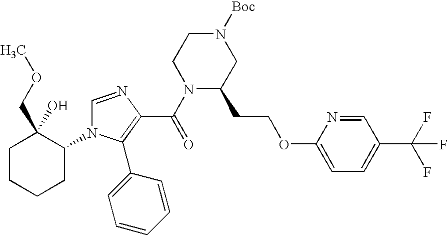

MS (ESI+, m/e) 688 (M+1)

### Reference Example 657

### 4-{2-[(2R)-4-[(Benzyloxy)carbonyl]-1-({1-[(1R,2S)-2-hydroxy-2-(methoxymethyl)cyclohexyl]-5-phenyl-1H-imidazol-4-yl}carbonyl)piperazin-2-yl]ethoxy}benzoic acid

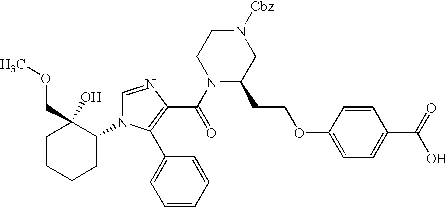

Benzyl (3R)-4-({1-[(1R,2S)-2-hydroxy-2-(methoxymethyl)cyclohexyl]-5-phenyl-1H-imidazol-4-yl}carbonyl)-3-{2-[4-(methoxycarbonyl)phenoxy]ethyl}piperazine-1-carboxylate (940 mg) was dissolved in methanol (50 ml), 1N aqueous sodium hydroxide solution (26.4 ml) was added, and the mixture was stirred at 60° C. for 15 hr. The reaction mixture was neutralized with 1N hydrochloric acid, and the solvent was concentrated under reduced pressure. The residue was diluted with ethyl acetate-THF (3:1), and the mixture was washed with saturated brine, dried over anhydrous magnesium sulfate, and concentrated under reduced pressure to give the object compound (896 mg).

MS (ESI+, m/e) 697 (M+1)

### Reference Example 658

### Benzyl (3R)-3-(2-{4-[(cyclopropylamino)carbonyl]phenoxy}ethyl)-4-({1-[(1R,2S)-2-hydroxy-2-(methoxymethyl)cyclohexyl]-5-phenyl-1H-imidazol-4-yl}carbonyl)piperazine-1-carboxylate

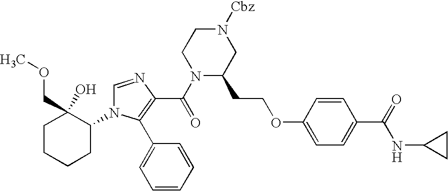

4-{2-[(2R)-4-[(Benzyloxy)carbonyl]-1-({1-[(1R,2S)-2-hydroxy-2-(methoxymethyl)cyclohexyl]-5-phenyl-1H-imidazol-4-yl}carbonyl)piperazin-2-yl]ethoxy}benzoic acid (139 mg) was suspended in DMF (3 ml), cyclopropylamine (23 mg), WSC.HCl (58 mg) and HOBt (37 mg) were added, and the mixture was stirred at room temperature for 12 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate was concentrated under reduced pressure to give the object compound (98 mg) as an amorphous solid.

MS (ESI+, m/e) 736 (M+1)

In the same manner as in Reference Example 658, the following compounds (Reference Examples 659-661) were obtained.

### Reference Example 659

### Benzyl (3R)-3-(2-{4-[(dimethylamino)carbonyl]phenoxy}ethyl)-4-({1-[(1R,2S)-2-hydroxy-2-(methoxymethyl)cyclohexyl]-5-phenyl-1H-imidazol-4-yl}carbonyl)piperazine-1-carboxylate

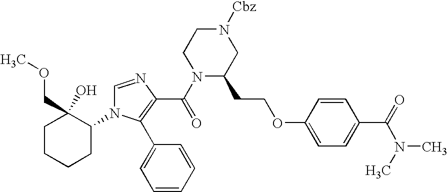

MS (ESI+, m/e) 724 (M+1)

### Reference Example 660

### Benzyl (3R)-3-{2-[4-(azetidin-1-ylcarbonyl)phenoxy]ethyl}-4-({1-[(1R,2S)-2-hydroxy-2-(methoxymethyl)cyclohexyl]-5-phenyl-1H-imidazol-4-yl}carbonyl)piperazine-1-carboxylate

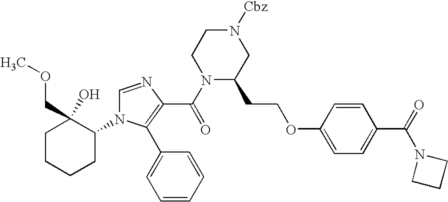

MS (ESI+, m/e) 736 (M+1)

### Reference Example 661

### Benzyl (3R)-4-({1-[(1R,2S)-2-hydroxy-2-(methoxymethyl)cyclohexyl]-5-phenyl-1H-imidazol-4-yl}carbonyl)-3-[2-(4-{[(2,2,2-trifluoroethyl)amino]carbonyl}phenoxy)ethyl]piperazine-1-carboxylate

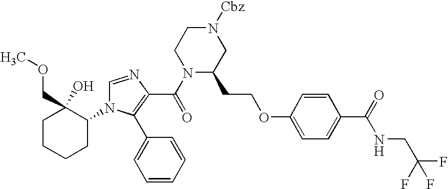

MS (ESI+, m/e) 778 (M+1)

### Reference Example 662

### 1-tert-Butyl 4-benzyl (2R)-2-(2-oxoethyl)piperazine-1,4-dicarboxylate

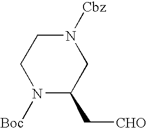

1-tert-Butyl 4-benzyl (2R)-2-(2-hydroxyethyl)piperazine-1,4-dicarboxylate (2.0 g) and triethylamine (2.3 ml) were dissolved in DMSO (20 ml), a solution of pyridine-sulfur trioxide complex (2.6 g) in DMSO (10 ml) was added thereto, and the mixture was stirred at room temperature for 3 hr. The reaction mixture was poured into ice water, and the mixture was extracted with ethyl acetate. The extract was washed successively with 10% aqueous citric acid solution, saturated aqueous sodium hydrogen carbonate and saturated brine, and dried over anhydrous sodium sulfate, and the solvent was evaporated under reduced pressure. The residue was subjected to silica gel column chromatography, and the fraction eluted with ethyl acetate-hexane (9:1) was concentrated under reduced pressure to give the object compound (1.9 g) as an oil.

¹H-NMR (CDCl₃) δ 1.45 (9H, s), 2.48-2.74 (2H, m), 2.82-3.18 (3H, m), 3.77-4.20 (3H, m), 4.54-4.84 (1H, m), 5.02-5.25 (2H, m), 7.21-7.51 (5H, m), 9.53-9.84 (1H, m)

### Reference Example 663

### 1-tert-Butyl 4-benzyl (2R)-2-(2-anilinoethyl)piperazine-1,4-dicarboxylate

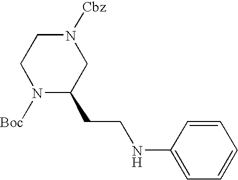

1-tert-Butyl 4-benzyl (2R)-2-(2-oxoethyl)piperazine-1,4-dicarboxylate (1.5 g) and aniline (1.1 g) were dissolved in dichloromethane-DMF (2:1, 30 ml), acetic acid (0.5 ml) was added, and the mixture was stirred for 30 min. Sodium triacetoxyborohydride (2.6 g) was added thereto, and the mixture was stirred at room temperature for 12 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate-hexane (1:1) was concentrated under reduced pressure to give the object compound (1.8 g) as an oil.

MS (ESI+, m/e) 440 (M+1)

### Reference Example 664

### 1-tert-Butyl 4-benzyl (2R)-2-{2-[methyl(phenyl)amino]ethyl}piperazine-1,4-dicarboxylate

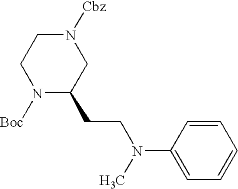

1-tert-Butyl 4-benzyl (2R)-2-(2-oxoethyl)piperazine-1,4-dicarboxylate (400 mg) and N-methylaniline (237 mg) were dissolved in dichloromethane-DMF (2:1, 8 ml), acetic acid (0.29 ml) was added, and the mixture was stirred for 30 min. Sodium triacetoxyborohydride (466 mg) was added thereto, and the mixture was stirred at room temperature for 15 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate-hexane (1:4-1:1) was concentrated under reduced pressure to give the object compound (370 mg) as an oil.

MS (ESI+, m/e) 454 (M+1)

### Reference Example 665

### Benzyl (3R)-3-(2-anilinoethyl)piperazine-1-carboxylate

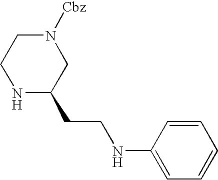

1-tert-Butyl 4-benzyl (2R)-2-(2-anilinoethyl)piperazine-1,4-dicarboxylate (380 mg) was dissolved in ethyl acetate (2 ml), 4N hydrogen chloride-ethyl acetate solution (5 ml) was added, and the mixture was stirred at room temperature for 1 hr. The reaction mixture was concentrated under reduced pressure, and the residue was suspended in ethyl acetate. The mixture was washed with saturated aqueous sodium hydrogen carbonate, and dried over anhydrous sodium sulfate, and the solvent was evaporated under reduced pressure to give the object compound (670 mg) as an oil.

MS (ESI+, m/e) 340 (M+1)

### Reference Example 666

### Benzyl (3R)-3-{2-[methyl(phenyl)amino]ethyl}piperazine-1-carboxylate

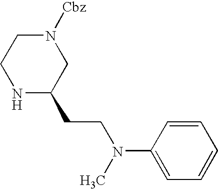

1-tert-Butyl 4-benzyl (2R)-2-{2-[methyl(phenyl)amino]ethyl}piperazine-1,4-dicarboxylate (380 mg) was dissolved in ethyl acetate (2 ml), 4N hydrogen chloride-ethyl acetate solution (5 ml) was added, and the mixture was stirred at room temperature for 1 hr. The reaction mixture was concentrated under reduced pressure, and the residue was suspended in ethyl acetate. The mixture was washed with saturated aqueous sodium hydrogen carbonate, and dried over anhydrous sodium sulfate, and the solvent was evaporated under reduced pressure to give the object compound (260 mg) as an oil.

MS (ESI+, m/e) 354 (M+1)

### Reference Example 667

### Ethyl 1-[(1R,2S)-2-ethyl-2-hydroxycyclohexyl]-5-phenyl-1H-imidazole-4-carboxylate

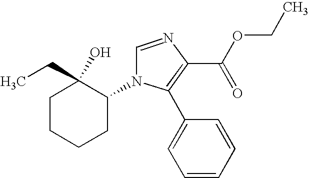

Copper iodide (4.57 g) was suspended in THF (100 ml), and the suspension was ice-cooled. Methylmagnesium bromide (1M THF solution, 45 ml) was added, and the mixture was stirred at 0° C. for 30 min. A solution of ethyl 1-[(3S,4R)-1-oxaspiro[2.5]oct-4-yl]-5-phenyl-1H-imidazole-4-carboxylate (4.90 g) in THF (50 ml) was added thereto, and the mixture was stirred at room temperature for 3 hr. To the reaction mixture was added saturated aqueous ammonium chloride solution, and the mixture was extracted with ethyl acetate. The extract was washed successively with saturated aqueous sodium hydrogen carbonate and saturated brine, dried over anhydrous magnesium sulfate, and concentrated under reduced pressure. The residue was subjected to silica gel column chromatography, and the fraction eluted with ethyl acetate was concentrated under reduced pressure to give the object compound (4.61 g) as an amorphous solid.

¹H-NMR (CDCl₃) δ 0.61 (3H, t), 0.90-1.32 (7H, m), 1.44-1.95 (7H, m), 2.12-2.33 (1H, m), 3.62 (1H, dd), 4.16-4.28 (1H, m), 7.19-7.37 (2H, m), 7.38-7.54 (3H, m), 8.04 (1H, s)

MS (ESI+, m/e) 389 (M+1)

In the same manner as in Reference Example 667, the following compound (Reference Example 668) was obtained.

### Reference Example 668

### Ethyl 1-[(1R,2R)-2-(cyclopropylmethyl)-2-hydroxycyclohexyl]-5-phenyl-1H-imidazole-4-carboxylate

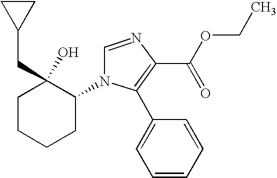

¹H-NMR (CDCl₃) δ −0.18-0.06 (2H, m), 0.25-0.50 (2H, m), 0.63-0.80 (1H, m), 1.07-1.33 (5H, m), 1.45-2.00 (8H, m), 2.24 (1H, dd), 3.45-3.71 (1H, m), 4.08-4.31 (2H, m), 7.12-7.36 (3H, m), 7.36-7.55 (2H, m), 8.03 (1H, s)

MS (ESI+, m/e) 369 (M+1)

### Reference Example 669

### Ethyl 1-[(1R,2S)-2-hydroxy-2-methylcyclohexyl]-5-phenyl-1H-imidazole-4-carboxylate

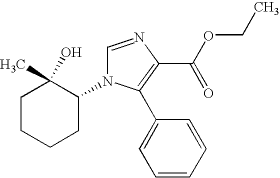

Ethyl 1-[(3S,4R)-1-oxaspiro[2.5]oct-4-yl]-5-phenyl-1H-imidazole-4-carboxylate (1.0 g) was dissolved in ethanol (30 ml), 20% palladium hydroxide-carbon (50% containing water, 100 mg) was added thereto, and the mixture was subjected to catalytic reduction at ambient temperature and normal pressure for 12 hr. The catalyst was filtered off, and the filtrate was concentrated under reduced pressure to give the object compound (972 mg) as an amorphous solid.

¹H-NMR (CDCl₃) δ 0.84 (3H, s), 1.22 (5H, t), 1.42-1.93 (6H, m), 2.19 (1H, dd), 3.56 (1H, dd), 4.12-4.29 (2H, m), 7.17-7.40 (2H, m), 7.41-7.53 (3H, m), 8.04 (1H, s)

MS (ESI+, m/e) 329 (M+1)

### Reference Example 670

### 1-[(1R,2S)-2-Ethyl-2-hydroxycyclohexyl]-5-phenyl-1H-imidazole-4-carboxylic acid

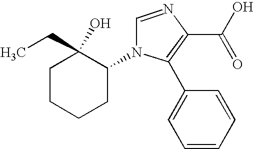

Ethyl 1-[(1R,2S)-2-ethyl-2-hydroxycyclohexyl]-5-phenyl-1H-imidazole-4-carboxylate (3.85 g) was dissolved in ethanol (30 ml), 4N aqueous sodium hydroxide solution (14 ml) was added thereto, and the mixture was stirred at 60° C. for 15 hr. After cooling to room temperature, the mixture was neutralized (pH 7) with diluted hydrochloric acid, and the solvent was evaporated under reduced pressure. The residue was suspended in THF (100 ml), and the insoluble material was filtered off. The filtrate was concentrated under reduced pressure to give the object compound (4.30 g) as a powder mixed with an inorganic salt thereof.

¹H-NMR (DMSO-d₆) δ 0.52 (3H, t), 0.60-1.22 (6H, m), 1.23-1.47 (1H, m), 1.50-1.90 (4H, m), 1.95-2.32 (1H, m), 7.35 (6H, m)

MS (ESI+, m/e) 315 (M+1)

In the same manner as in Reference Example 670, the following compounds (Reference Examples 671-672) were obtained.

### Reference Example 671

### 1-[(1R,2R)-2-(Cyclopropylmethyl)-2-hydroxycyclohexyl]-5-phenyl-1H-imidazole-4-carboxylic acid

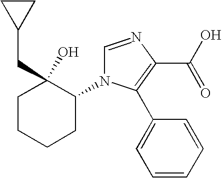

¹H-NMR (CDCl₃) δ −0.16 (2H, br s), 0.08-0.39 (3H, m), 0.66-1.05 (2H, m), 1.05-1.34 (2H, m), 1.43 (1H, s), 1.53-1.97 (4H, m), 2.03-2.30 (1H, m), 3.48-3.67 (1H, m), 3.68-3.84 (1H, m), 7.10-7.44 (5H, m), 7.95 (1H, s)

MS (ESI+, m/e) 341 (M+1)

### Reference Example 672

### 1-[(1R,2S)-2-Hydroxy-2-methylcyclohexyl]-5-phenyl-1H-imidazole-4-carboxylic acid

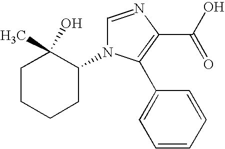

¹H-NMR (DMSO-d₆) δ 0.66 (3H, s), 0.97-1.23 (2H, m), 1.28-1.43 (1H, m), 1.49-1.88 (4H, m), 2.16 (1H, tq), 3.39-3.56 (1H, m), 3.55-3.68 (1H, m), 7.30-7.44 (2H, m), 7.46-7.58 (3H, m), 8.34-8.52 (1H, m)

MS (ESI+, m/e) 301 (M+1)

### Reference Example 673

### 1-[(1R,2S)-2-(Ethoxymethyl)-2-hydroxycyclohexyl]-5-phenyl-1H-imidazole-4-carboxylic acid

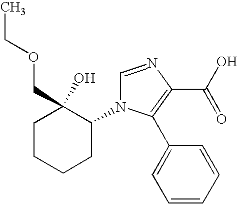

Ethyl 1-[(3S,4R)-1-oxaspiro[2.5]oct-4-yl]-5-phenyl-1H-imidazole-4-carboxylate (1.96 g) was dissolved in ethanol (30 ml), sodium ethoxide (20% ethanol solution, 11.8 ml) was added thereto at room temperature, and the mixture was stirred at 60° C. for 3 hr. 1N Aqueous sodium hydroxide solution (6 ml) was added thereto, and the mixture was further stirred at 60° C. for 3 hr. After cooling to room temperature, the mixture was neutralized (pH 7) with diluted hydrochloric acid, and the solvent was evaporated under reduced pressure. The residue was suspended in ethanol (100 ml), and the insoluble material was filtered off. The filtrate was concentrated under reduced pressure to give the object compound (2.28 g) as a powder mixed with an inorganic salt thereof.

¹H-NMR (CDCl₃) δ 1.00 (3H, t), 1.12-1.29 (2H, m), 1.41-1.54 (1H, m), 1.59-1.71 (1H, m), 1.71-1.83 (3H, m), 2.11-2.27 (1H, m), 2.80 (2H, s), 3.26 (2H, q), 3.66-3.82 (1H, m), 4.29 (1H, br s), 7.23-7.36 (2H, m), 7.38-7.46 (3H, m), 8.12 (1H, s)

MS (ESI+, m/e) 345 (M+1)

In the same manner as in Reference Example 86, the following compounds (Reference Examples 674-676) were obtained.

### Reference Example 674

### Ethyl N-(tert-butoxycarbonyl)-DL-2-methoxyphenylalanyl-N-benzylglycinate

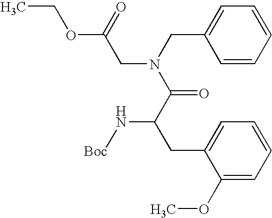

¹H-NMR (CDCl₃) δ 1.19-1.72 (12H, m), 2.50-3.31 (2H, m), 3.64-3.90 (3H, m), 4.00-4.27 (3H, m), 4.48-5.46 (3H, m), 6.75-6.92 (2H, m), 7.01-7.42 (7H, m)

### Reference Example 675

### Ethyl N-(tert-butoxycarbonyl)-D-(biphenyl-4-yl)alanyl-N-benzylglycinate

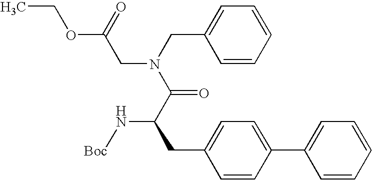

MS (ESI+, m/e) 417 (M+1-“Boc”)

### Reference Example 676

### Ethyl 2-bromo-N-(tert-butoxycarbonyl)-D-phenylalanyl-N-benzylglycinate

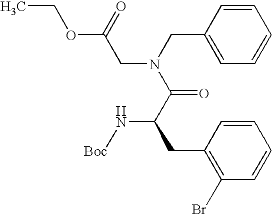

MS (ESI+, m/e) 519 (M+1)

In the same manner as in Reference Example 109, the following compounds (Reference Examples 677-679) were obtained.

### Reference Example 677

### 1-Benzyl-3-(2-methoxybenzyl)piperazine-2,5-dione

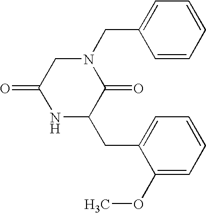

¹H-NMR (DMSO-d₆) δ 2.90-3.00 (1H, m), 3.04-3.19 (2H, m), 3.47 (1H, d), 3.74 (3H, s), 4.08-4.15 (1H, m), 4.43 (2H, s), 6.64-6.76 (4H, m), 6.96 (2H, dd), 8.09 (1H, br s)

### Reference Example 678

### (3R)-1-Benzyl-3-(biphenyl-4-ylmethyl)piperazine-2,5-dione

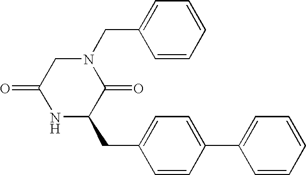

¹H-NMR (CDCl₃) δ 3.06 (1H, d), 3.21 (2H, d), 3.54 (1H, d), 4.35-4.40 (1H, m), 4.43 (1H, d), 4.55 (1H, d), 6.49 (1H, s), 7.16-7.53 (14H, m)

MS (ESI+, m/e) 371 (M+1)

### Reference Example 679

### (3R)-1-Benzyl-3-(2-bromobenzyl)piperazine-2,5-dione

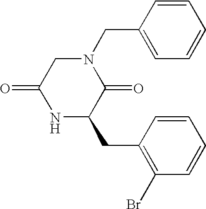

MS (ESI+, m/e) 373 (M+1)

In the same manner as in Reference Example 133, the following compounds (Reference Examples 680-681) were obtained.

### Reference Example 680

### 1-Benzyl-3-(2-methoxybenzyl)piperazine

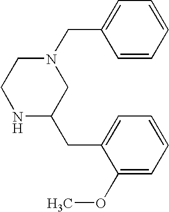

¹H-NMR (CDCl₃) δ 2.51-3.10 (9H, m), 3.40-3.61 (2H, m), 3.66-3.74 (1H, m), 3.80 (3H, s), 6.80-6.93 (4H, m), 7.09-7.36 (5H, m)

MS (ESI+, m/e) 297 (M+1)

### Reference Example 681

### (3R)-1-Benzyl-3-(biphenyl-4-ylmethyl)piperazine

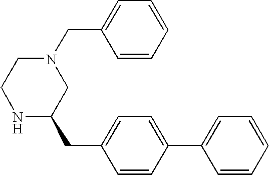

¹H-NMR (CDCl₃) δ 1.64 (1H, br s), 1.92 (1H, t), 2.10 (1H, dt), 2.57 (1H, dd), 2.72-2.94 (5H, m), 2.98-3.07 (1H, m), 3.48 (1H, d), 3.55 (1H, d), 7.21-7.58 (14H, m)

MS (ESI+, m/e) 343 (M+1)

In the same manner as in Reference Example 147, the following compound (Reference Example 682) was obtained.

### Reference Example 682

### (3R)-1-Benzyl-3-(2-bromobenzyl)piperazine

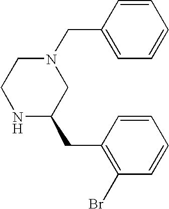

MS (ESI+, m/e) 345 (M+1)

### Reference Example 683

### tert-Butyl [(1R)-1-benzyl-2-(benzylamino)propyl]carbamate

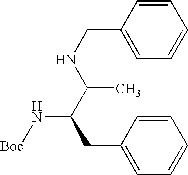

tert-Butyl [(1R)-1-benzyl-2-oxopropyl]carbamate (3.42 g) was dissolved in 1,2-dichloroethane (50 ml), benzylamine (1.39 g) and acetic acid (780 mg) were added, and the mixture was stirred at room temperature for 15 min. Sodium triacetoxyborohydride (3.6 g) was added thereto, and the mixture was further stirred at room temperature for 15 hr. The reaction mixture was neutralized with saturated aqueous sodium hydrogen carbonate, and the organic layer was dried over anhydrous sodium sulfate, and concentrated under reduced pressure. The residue was subjected to silica gel column chromatography, and the fraction eluted with ethyl acetate-hexane (1:2) was concentrated under reduced pressure to give the object compound (4.40 g) as an oil.

MS (ESI+, m/e) 355 (M+1)

### Reference Example 684

### Ethyl N-benzyl-N-{(2R)-2-[(tert-butoxycarbonyl)amino]-1-methyl-3-phenylpropyl}glycinate

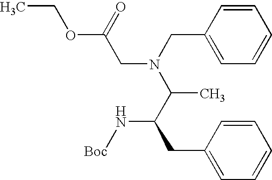

A mixture of tert-butyl [(1R)-1-benzyl-2-(benzylamino)propyl]carbamate (1.06 g), potassium carbonate (498 mg), ethyl bromoacetate (501 mg) and ethanol (10 ml) was stirred at 50° C. for 5 hr, and the solvent was evaporated under reduced pressure. The residue was poured into water, and the mixture was extracted with ethyl acetate. The extract was washed successively with water and saturated brine, and dried over anhydrous magnesium sulfate, and the solvent was evaporated under reduced pressure. The residue was subjected to silica gel column chromatography, and the fraction eluted with ethyl acetate-hexane (1:2) was concentrated under reduced pressure to give the object compound (430 mg) as an oil.

MS (ESI+, m/e) 441 (M+1)

### Reference Example 685

### (6R)-4,6-Dibenzyl-5-methylpiperazin-2-one

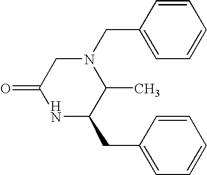

Ethyl N-benzyl-N-{(2R)-2-[(tert-butoxycarbonyl)amino]-1-methyl-3-phenylpropyl}glycinate (419 mg) was dissolved in dichloromethane (1 ml), TFA (2 ml) was added thereto, and the mixture was stirred at room temperature for 30 min. The solvent was evaporated under reduced pressure, and the residue was dissolved in THF (5 ml). Triethylamine (1 ml) was added thereto at room temperature, and the mixture was stirred at room temperature for 15 hr. The solvent was evaporated under reduced pressure, and the residue was dissolved in ethyl acetate. The mixture was washed with saturated brine, and dried over anhydrous magnesium sulfate, and the solvent was evaporated under reduced pressure to give the object compound (270 mg) as an oil.

MS (ESI+, m/e) 295 (M+1)

### Reference Example 686

### (3R)-1,3-Dibenzyl-2-methylpiperazine

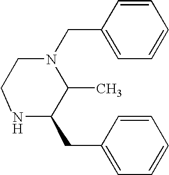

A mixture of (6R)-4,6-dibenzyl-5-methylpiperazin-2-one (265 mg) and THF (6 ml) was ice-cooled, and lithium aluminum hydride (68 mg) was added by small portions. The mixture was stirred at room temperature for 30 min, and then at 60° C. for 3 hr. The mixture was cooled to −78° C., and ethanol-ethyl acetate (1:1, 2 ml) and 1N aqueous sodium hydroxide solution (2 ml) were successively added dropwise. After the completion of the dropwise addition, the mixture was stirred at room temperature for 40 min. The insoluble material was filtered, and washed with ethyl acetate. The filtrate was washed with saturated brine, and dried over anhydrous magnesium sulfate, and the solvent was evaporated under reduced pressure to give the object compound (240 mg) as an oil.

MS (ESI+, m/e) 281 (M+1)

In the same manner as in Reference Example 157, the following compound (Reference Example 687) was obtained.

### Reference Example 687

### tert-Butyl (2R)-4-benzyl-2-(2-bromobenzyl)piperazine-1-carboxylate

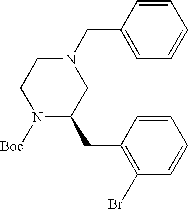

MS (ESI+, m/e) 445 (M+1)

In the same manner as in Reference Example 158, the following compound (Reference Example 688) was obtained.

### Reference Example 688

### tert-Butyl (2R)-4-benzyl-2-(2-morpholinobenzyl)piperazine-1-carboxylate

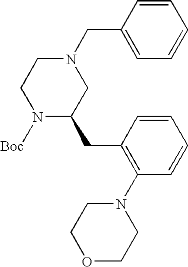

MS (ESI+, m/e) 452 (M+1)

### Reference Example 689

### tert-Butyl (2R)-4-benzyl-2-[2-(2-methoxypyridin-3-yl)benzyl]piperazine-1-carboxylate

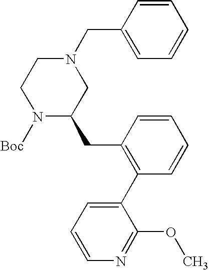

tert-Butyl (2R)-4-benzyl-2-(2-bromobenzyl)piperazine-1-carboxylate (445 mg), (2-methoxypyridin-3-yl)boronic acid (184 mg), tetrakis(triphenylphosphine)palladium(0) (58 mg) and sodium carbonate (254 mg) were suspended in 1,2-dimethoxyethane-water (2:1, 9 ml), and the suspension was stirred at 100° C. for 15 hr. Ethyl acetate was added to the reaction mixture, and the insoluble material was filtered off. The filtrate was washed with saturated brine, and dried over anhydrous magnesium sulfate, and the solvent was evaporated under reduced pressure. The residue was subjected to silica gel column chromatography, and the fraction eluted with ethyl acetate-hexane (1:3) was concentrated under reduced pressure to give the object compound (145 mg) as an oil.

MS (ESI+, m/e) 473 (M+1)

### Reference Example 690

### tert-Butyl (2R)-4-benzyl-2-[(6-chloropyridin-2-yl)benzyl]piperazine-1-carboxylate

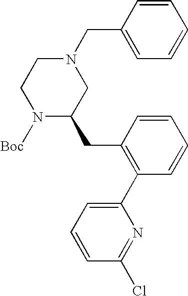

A mixture of tert-butyl (2R)-4-benzyl-2-(2-bromobenzyl)piperazine-1-carboxylate (445 mg), 2-chloro-6-(tributylstannyl)pyridine (426 mg), tetrakis(triphenylphosphine)palladium(0) (58 mg) and toluene (5 ml) was stirred at 100° C. for 15 hr. The insoluble material was filtered off, and the filtrate was concentrated under reduced pressure. The residue was subjected to basic silica gel column chromatography, and the fraction eluted with ethyl acetate-hexane (1:3) was concentrated under reduced pressure to give the object compound (140 mg) as an oil.

MS (ESI+, m/e) 478 (M+1)

### Reference Example 691

### tert-Butyl (2R)-4-benzyl-2-[2-(4,4,5,5-tetramethyl-1,3,2-dioxaborolan-2-yl)benzyl]piperazine-1-carboxylate

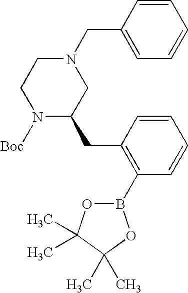

tert-Butyl (2R)-4-benzyl-2-(2-bromobenzyl)piperazine-1-carboxylate (890 mg), bis(pinacolato)diboron (584 mg), [1,1′-bis(diphenylphosphino)ferrocene]dichloropalladium(II) dichloromethane adduct (65 mg) and potassium acetate (590 mg) were dissolved in DMF (10 ml), and the solution was stirred at 120° C. for 20 hr. Ethyl acetate-water (2:1) was added to the reaction mixture, and the insoluble material was filtered off. The filtrate was washed with saturated brine, and dried over anhydrous magnesium sulfate, and the solvent was evaporated under reduced pressure. The residue was subjected to silica gel column chromatography, and the fraction eluted with ethyl acetate-hexane (1:2) was concentrated under reduced pressure to give the object compound (500 mg) as an oil.

MS (ESI+, m/e) 4.93 (M+1)

### Reference Example 692

### tert-Butyl (2R)-4-benzyl-2-(2-hydroxybenzyl)piperazine-1-carboxylate

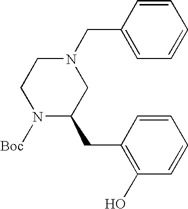

tert-Butyl (2R)-4-benzyl-2-[2-(4,4,5,5-tetramethyl-1,3,2-dioxaborolan-2-yl)benzyl]piperazine-1-carboxylate (420 mg) was dissolved in acetone (4 ml), and a solution of potassium peroxymonosulfate (524 mg) in water (4 ml) was added at room temperature. The mixture was stirred at room temperature for 10 min, saturated aqueous sodium thiosulfate solution (4 ml) was added, and the liberated oil was extracted with ethyl acetate. The extract was washed successively with water and saturated brine, and dried over anhydrous magnesium sulfate, and the solvent was evaporated under reduced pressure. The residue was subjected to silica gel column chromatography, and the fraction eluted with ethyl acetate-hexane (1:3) was concentrated under reduced pressure to give the object compound (300 mg) as an oil.

MS (ESI+, m/e) 383 (M+1)

### Reference Example 693

### tert-Butyl (2R)-4-benzyl-2-(2-phenoxybenzyl)piperazine-1-carboxylate

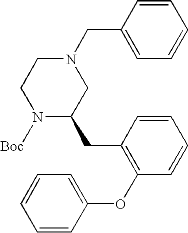

tert-Butyl (2R)-4-benzyl-2-(2-hydroxybenzyl)piperazine-1-carboxylate (300 mg), phenylboronic acid (95 mg), copper(II) acetate (283 mg), pyridine (308 mg), triethylamine (395 mg) and pulverized molecular sieves 4 A (600 mg) were suspended in dichloromethane, and the suspension was stirred at room temperature for 20 hr. The insoluble material was filtered off, and the filtrate was concentrated under reduced pressure. The residue was subjected to silica gel column chromatography, and the fraction eluted with ethyl acetate-hexane (1:3) was concentrated under reduced pressure to give the object compound (150 mg) as an oil.

MS (ESI+, m/e) 459 (M+1)

### Reference Example 694

### tert-Butyl (2R)-4-benzyl-2-[2-(1-methyl-1H-pyrazol-5-yl)benzyl]piperazine-1-carboxylate

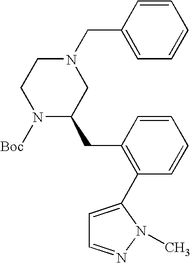

A mixture of tert-butyl (2R)-4-benzyl-2-(2-bromobenzyl)piperazine-1-carboxylate (445 mg), 1-methyl-5-(tributylstannyl)-1H-pyrazole (426 mg), tetrakis(triphenylphosphine)palladium(0) (58 mg) and toluene (5 ml) was stirred at 100° C. for 12 hr. The insoluble material was filtered off, and the filtrate was concentrated under reduced pressure. The residue was subjected to basic silica gel column chromatography, and the fraction eluted with ethyl acetate-hexane (1:3) was concentrated under reduced pressure to give the object compound (240 mg) as an oil.

MS (ESI+, m/e) 447 (M+1)

In the same manner as in Reference Example 243, the following compounds (Reference Examples 695-696) were obtained.

### Reference Example 695

### tert-Butyl (3S)-3-[(2-phenyl-1H-imidazol-1-yl)methyl]piperazine-1-carboxylate

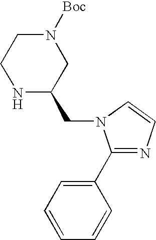

MS (ESI+, m/e) 343 (M+1)

### Reference Example 696

### tert-Butyl (3S)-3-[(5-phenyl-1H-imidazol-1-yl)methyl]piperazine-1-carboxylate

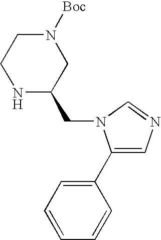

MS (ESI+, m/e) 343 (M+1)

### Reference Example 697

### 1-Benzyl-3-(biphenyl-2-ylmethyl)piperazine-2,5-dione

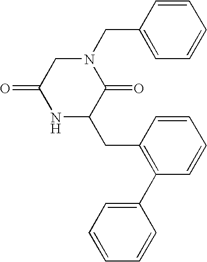

A mixture of (3R)-1-benzyl-3-(2-bromobenzyl)piperazine-2,5-dione (500 mg), phenylboronic acid (250 mg), tetrakis(triphenylphosphine)palladium(0) (231 mg), sodium carbonate (355 mg), DME(7 ml) and water (3.5 ml) was heated under reflux for 12 hr, and concentrated under reduced pressure. The residue was suspended in ethyl acetate, and the suspension was washed with 10% aqueous sodium bicarbonate. The organic layer was dried over anhydrous sodium sulfate, and the solvent was evaporated under reduced pressure. The residue was subjected to silica gel column chromatography, and the fraction eluted with ethyl acetate-hexane (1:4-1:0) was concentrated under reduced pressure. The crystals were collected by filtration to give the object compound (290 mg) (The racemization proceeded in the course of the reaction.)

MS (ESI+, m/e) 371 (M+1)

In the same manner as in Reference Example 133, the following compound (Reference Example 698) was obtained.

### Reference Example 698

### 1-Benzyl-3-(biphenyl-2-ylmethyl)piperazine

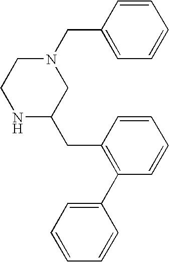

MS (ESI+, m/e) 343 (M+1)

### Reference Example 699

### Benzyl (3S)-3-(hydroxymethyl)-4-tritylpiperazine-1-carboxylate

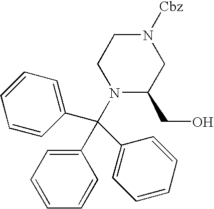

Benzyl (3S)-3-(hydroxymethyl)piperazine-1-carboxylate (8.36 g) and triethylamine (9.3 ml) were dissolved in DMF (50 ml), and the solution was ice-cooled. Trityl chloride (9.78 g) was added, and the mixture was stirred at 0° C. for 1 hr, and then at room temperature for 15 hr. To the reaction mixture was added saturated aqueous sodium hydrogen carbonate, and the mixture was extracted with ethyl acetate. The extract was washed with saturated brine, dried over anhydrous magnesium sulfate, and concentrated under reduced pressure. The residue was subjected to silica gel column chromatography, and the fraction eluted with ethyl acetate-hexane (1:1) was concentrated under reduced pressure to give the object compound (8.54 g) as an amorphous solid.

MS (ESI+, m/e) 493 (M+1)

### Reference Example 700

### Benzyl (3S)-3-{[(3-fluorophenyl)thio]methyl}-4-tritylpiperazine-1-carboxylate

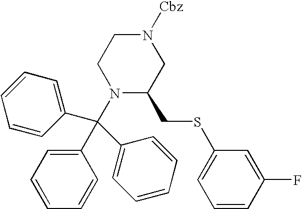

Benzyl (3S)-3-(hydroxymethyl)-4-tritylpiperazine-1-carboxylate (985 mg), 3-fluorothiophenol (308 mg) and tri-tert-butylphosphine (486 mg) were dissolved in toluene (40 ml), ADDP (757 mg) was added, and the mixture was stirred at room temperature for 12 hr. The reaction mixture was poured into saturated aqueous sodium hydrogen carbonate, and the mixture was extracted with ethyl acetate. The extract was washed with saturated brine, and dried over anhydrous sodium sulfate, and the solvent was evaporated under reduced pressure. The residue was subjected to silica gel column chromatography, and the fraction eluted with ethyl acetate-hexane (1:1) was concentrated under reduced pressure to give the object compound (600 mg) as an amorphous solid.

MS (ESI+, m/e) 361 (M+1-“Tr”)

### Reference Example 701

### Benzyl (3S)-3-{[(3-fluorophenyl)sulfonyl]methyl}piperazine-1-carboxylate

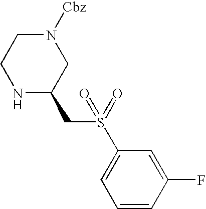

Benzyl (3S)-3-{[(3-fluorophenyl)thio]methyl}-4-tritylpiperazine-1-carboxylate (600 mg) was dissolved in dichloromethane (10 ml), and the solution was ice-cooled. mCPBA (542 mg) was added, and the mixture was stirred at 0° C. for 30 min. To the reaction mixture was added aqueous sodium thiosulfate solution, and the mixture was extracted with ethyl acetate. The extract was washed successively with saturated aqueous sodium hydrogen carbonate and saturated brine, and dried over anhydrous sodium sulfate, and the solvent was evaporated under reduced pressure to give benzyl (3S)-3-{[(3-fluorophenyl)sulfonyl]methyl}-4-tritylpiperazine-1-carboxylate (624 mg) as an amorphous solid. The total amount thereof was dissolved in ethyl acetate (5 ml), 4N hydrogen chloride-ethyl acetate solution (5 ml) was added, and the mixture was stirred at room temperature for 5 hr. The reaction mixture was concentrated under reduced pressure, the residue was diluted with saturated aqueous sodium hydrogen carbonate, and the mixture was extracted with ethyl acetate. The extract was washed with saturated brine, dried over anhydrous magnesium sulfate, and concentrated under reduced pressure. The residue was subjected to basic silica gel column chromatography, and the fraction eluted with ethyl acetate-methanol (9:1) was concentrated under reduced pressure to give the object compound (250 mg) as an oil.

MS (ESI+, m/e) 393 (M+1)

### Reference Example 702

### Benzyl (3S)-3-formyl-4-tritylpiperazine-1-carboxylate

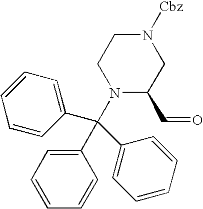

Benzyl (3S)-3-(hydroxymethyl)-4-tritylpiperazine-1-carboxylate (985 mg) and triethylamine (836 μl) were dissolved in DMSO (10 ml), a solution of sulfur trioxide pyridine complex (955 mg) in DMSO (5 ml) was added while cooling in water bath, and the mixture was stirred at room temperature for 2 hr. The reaction mixture was poured into water, and the mixture was extracted with ethyl acetate. The extract was washed successively with saturated aqueous sodium hydrogen carbonate and saturated brine, and dried over anhydrous sodium sulfate, and the solvent was evaporated under reduced pressure. The residue was subjected to silica gel column chromatography, and the fraction eluted with ethyl acetate was concentrated under reduced pressure. The crystals were collected by filtration to give the object compound (591 mg).

¹H-NMR (CDCl₃) δ 2.94-3.38 (4H, m), 3.71-4.06 (2H, m), 4.24-4.38 (1H, m), 5.03 (2H, s), 7.12-7.37 (14H, m), 7.39-7.64 (6H, m), 8.51 (1H, br s)

### Reference Example 703

### Benzyl (3R)-3-{[(2-ethyl-1,3-benzoxazol-5-yl)amino]methyl}piperazine-1-carboxylate

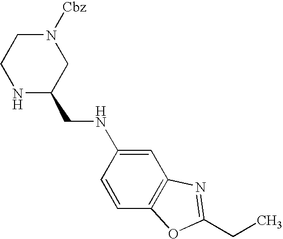

Benzyl (3S)-3-formyl-4-tritylpiperazine-1-carboxylate (295 mg) and 2-ethyl-1,3-benzoxazole-5-amine (97 mg) were dissolved in 1,2-dichloroethane (5 ml), acetic acid (0.25 ml) and sodium triacetoxyborohydride (191 mg) were added, and the mixture was stirred at room temperature for 15 hr. 4N Hydrogen chloride-ethyl acetate solution (2 ml) was added, and the mixture was stirred at room temperature for 15 hr. To the reaction mixture was added saturated aqueous sodium hydrogen carbonate, and the mixture was extracted with ethyl acetate. The extract was washed with saturated brine, dried over anhydrous magnesium sulfate, and concentrated under reduced pressure. The residue was subjected to silica gel column chromatography, and the fraction eluted with ethyl acetate was concentrated under reduced pressure to give the object compound (128 mg) as an oil.

MS (ESI+, m/e) 395 (M+1)

### Reference Example 704

### 4-[4-(Benzyloxy)-3-methoxyphenyl]morpholine

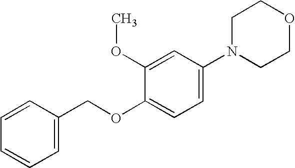

A mixture of 1-(benzyloxy)-4-bromo-2-methoxybenzene (1.47 g), morpholine (479 mg), BINAP (311 mg), sodium tert-butoxide (721 mg), Pd₂(dba)₃ (183 mg) and toluene (45 ml) was stirred at 90° C. for 15 hr in an argon stream, and poured into saturated aqueous sodium hydrogen carbonate, and the mixture was extracted with ethyl acetate-THF (3:1) (along with which the insoluble material was filtered off). The extract was washed successively with water and saturated brine, and dried over anhydrous magnesium sulfate, and the solvent was evaporated under reduced pressure. The residue was subjected to silica gel column chromatography, and the fraction eluted with ethyl acetate-hexane (1:6-1:2) was concentrated under reduced pressure, and the crystals were collected by filtration to give the object compound (1.13 g).

¹H-NMR (CDCl₃) δ 3.05-3.08 (4H, m), 3.83-3.86 (4H, m), 3.87 (3H, s), 5.08 (2H, s), 6.37 (1H, dd), 6.55 (1H, d), 6.80 (1H, d), 7.28-7.44 (5H, m)

MS (ESI+, m/e) 300 (M+1)

In the same manner as in Reference Example 704, the following compound (Reference Example 705) was obtained.

### Reference Example 705

### 1-Acetyl-4-[4-(benzyloxy)-3-methoxyphenyl]piperazine

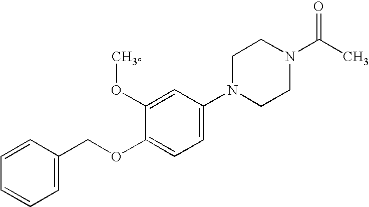

¹H-NMR (CDCl₃) δ 2.13 (3H, s), 3.02-3.08 (4H, m), 3.61 (2H, t), 3.76 (2H, t), 3.88 (3H, s), 5.09 (2H, s), 6.38 (1H, dd), 6.57 (1H, d), 6.80 (1H, d), 7.28-7.44 (5H, m)

MS (ESI+, m/e) 341 (M+1)

### Reference Example 706

### 2-Methoxy-4-morpholinophenol

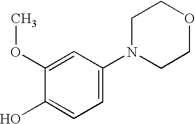

4-[4-(Benzyloxy)-3-methoxyphenyl]morpholine (1.12 g) was dissolved in methanol-THF (3:1, 40 ml), 20% palladium hydroxide-carbon (50% containing water, 560 mg) was added thereto, and the mixture was subjected to catalytic reduction at ambient temperature and normal pressure for 15 hr. The catalyst was filtered off, and the filtrate was concentrated under reduced pressure. The residue was dissolved in ethyl acetate, and the solution was dried over anhydrous magnesium sulfate. The solvent was evaporated under reduced pressure, and the crystals were collected by filtration to give the object compound (684 mg).

¹H-NMR (CDCl₃) δ 3.04-3.07 (4H, m), 3.85-3.87 (4H, m), 3.87 (3H, s), 5.30 (1H, br s), 6.44 (1H, dd), 6.54 (1H, s), 6.83 (1H, d)

MS (ESI+, m/e) 210 (M+1)

In the same manner as in Reference Example 706, the following compound (Reference Example 707) was obtained.

### Reference Example 707

### 4-(4-Acetylpiperazin-1-yl)-2-methoxyphenol

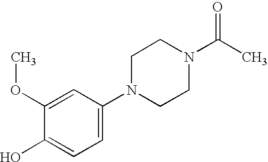

¹H-NMR (CDCl₃) δ 2.14 (3H, s), 3.00-3.06 (4H, m), 3.61 (2H, t), 3.77 (2H, t), 3.87 (3H, s), 5.41 (1H, br s), 6.44 (1H, dd), 6.54 (1H, d), 6.83 (1H, d)

MS (ESI+, m/e) 251 (M+1)

### Reference Example 708

### 4-Bromo-2-fluoro-1-[(2-methyl-2-propen-1-yl)oxy]benzene

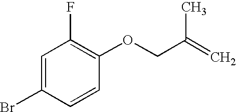

4-Bromo-2-fluorophenol (26.8 g) and 3-chloro-2-methyl-1-propene (13.7 ml) were dissolved in acetone (420 ml), potassium carbonate (29.0 g) was added thereto, and the mixture was heated under reflux for 15 hr. The reaction mixture was poured into water, and the mixture was extracted with ethyl acetate. The extract was washed with saturated brine, and dried over anhydrous sodium sulfate, and the solvent was evaporated under reduced pressure. The residue was subjected to silica gel column chromatography, and the fraction eluted with ethyl acetate-hexane (1:4) was concentrated under reduced pressure to give the object compound (29.9 g).

¹H-NMR (CDCl₃) δ 1.83 (3H, s), 4.48 (2H, s), 5.04 (2H, d), 6.84 (1H, t), 7.13 (2H, m)

### Reference Example 709

### 5-Bromo-7-fluoro-2,2-dimethyl-2,3-dihydro-1-benzofuran

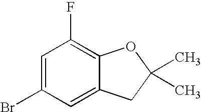

A mixture of 4-bromo-2-fluoro-1-[(2-methyl-2-propen-1-yl)oxy]benzene (29.9 g) and N,N-diethylaniline (30 ml) was stirred at 190° C. for 5 hr. The mixture was cooled to room temperature, and diisopropyl ether was added thereto. The mixture was washed successively with 1N hydrochloric acid, water and saturated brine, and dried over anhydrous magnesium sulfate, and the solvent was evaporated under reduced pressure. The residue was dissolved in toluene (240 ml), boron trifluoride diethyl ether complex (30 ml) was added thereto, and the mixture was stirred at 60° C. for 15 hr. The reaction mixture was poured into ice water, and the mixture was extracted with ethyl acetate. The extract was washed with saturated brine, and dried over anhydrous sodium sulfate, and the solvent was evaporated under reduced pressure. The residue was subjected to silica gel column chromatography, and the fraction eluted with ethyl acetate-hexane (1:4) was concentrated under reduced pressure to give the object compound (18.9 g).

¹H-NMR (CDCl₃) δ 1.51 (6H, s), 3.04 (2H, s), 6.97-7.24 (2H, m)

### Reference Example 710

### (7-Fluoro-2,2-dimethyl-2,3-dihydro-1-benzofuran-5-yl)boronic acid

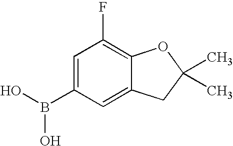

5-Bromo-7-fluoro-2,2-dimethyl-2,3-dihydro-1-benzofuran (18.9 g) was dissolved in THF (250 ml), and the solution was cooled to −78° C. n-Butyllithium (1.6M hexane solution, 53 ml) was added, and the mixture was stirred at −78° C. for 1 hr. Triisopropyl borate (21 ml) was added thereto at −78° C., and the mixture was stirred at room temperature for 12 hr. To the reaction mixture was added 1N hydrochloric acid (150 ml), and the mixture was stirred at room temperature for 3 hr, and extracted with ethyl acetate. The extract was washed successively with water, saturated aqueous sodium hydrogen carbonate and saturated brine, and dried over anhydrous sodium sulfate, and the solvent was evaporated under reduced pressure. The residue was subjected to silica gel column chromatography, and the fraction eluted with ethyl acetate-hexane (1:1) was concentrated under reduced pressure to give the object compound (6.54 g).

¹H-NMR (DMSO-d₆) δ 1.16-1.35 (2H, m), 1.45 (6H, s), 7.26-7.50 (2H, m), 7.90 (2H, br s)

### Reference Example 711

### 7-Fluoro-2,2-dimethyl-2,3-dihydro-1-benzofuran-5-ol

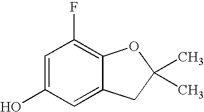

(7-Fluoro-2,2-dimethyl-2,3-dihydro-1-benzofuran-5-yl)boronic acid (1.2 g) was dissolved in acetone (20 ml), a solution of OXONE (3.7 g) in water (20 ml) was added dropwise at room temperature, and the mixture was stirred for 10 min. To the reaction mixture was added 10% aqueous sodium thiosulfate solution (100 ml), and the mixture was stirred for 30 min. The solvent was evaporated under reduced pressure, and the aqueous layer was extracted with ethyl acetate. The extract was washed with saturated brine, and dried over anhydrous sodium sulfate, and the solvent was evaporated under reduced pressure. The residue was subjected to silica gel column chromatography, and the fraction eluted with ethyl acetate-hexane (1:3) was concentrated under reduced pressure to give the object compound (600 mg).

¹H-NMR (DMSO-d₆) δ 1.50 (2H, m), 3.00 (6H, s), 4.86 (1H, br s), 6.41-6.50 (2H, m)

In the same manner as in Reference Example 255, the following compounds (Reference Examples 712-719) shown in Table 9 were obtained. In the column of “MS (ESI+)” in the Table, “*” means that a mass value of “M+1-“Boc”” was obtained (a mass value of M+1 was obtained for other compounds).

TABLE 9

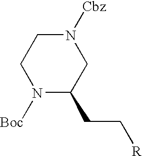

| Ref. Ex. No. | R | Compound | MS(ESI+) |
|---|---|---|---|
| 712 | 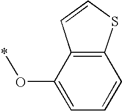 | 1-tert-Butyl 4-benzyl (2R)-2-[2-(1- benzothien-4-yloxy}ethyl] piperazine-1,4-dicarboxylate | 497 |
| 713 | 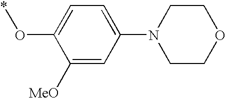 | 1-tert-Butyl 4-benzyl (2R)-2-[2-(2- methoxy-4-morpholinophenoxy) ethyl]piperazine-1,4-dicarboxylate | 556 |
| 714 | 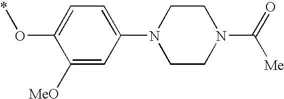 | 1-tert-Butyl 4-benzyl (2R)-2-{2-[4- (4-acetylpiperazin-1-yl)-2- methoxyphenoxy]ethyl}piperazine- 1,4-dicarboxylate | 597 |
| 715 | 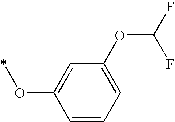 | 1-tert-Butyl 4-benzyl (2R)-2-{2-(3- (difluoromethoxy)phenoxy]ethyl} piperazine-1,4-dicarboxylate | 407* |
| 716 | 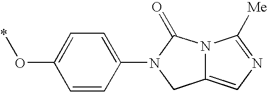 | 1-tert-Butyl 4-benzyl (2R)-2-{2-[4- (5-methyl-3-oxo-1H-imidazo[1,5-c] imidazol-2(3H)-yl)phenoxy]ethyl} piperazine-1,4-dicarboxylate | 576 |
| 717 | 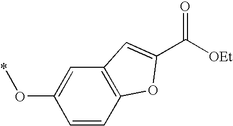 | 1-tert-Butyl 4-benzyl (2R)-2-(2-{[2- (ethoxycarbonyl)-1-benzofuran-5-yl] oxy}ethyl)piperazine-1,4- dicarboxylate | 553 |
| 718 | 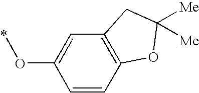 | 1-tert-Butyl 4-benzyl (2R)-2-{2- [(2,2-dimethyl-2,3-dihydro-1- benzofuran-5-yl)oxy]ethyl} piperazine-1,4-dicarboxylate | 511 |
| 719 | 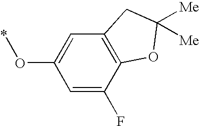 | 1-tert-Butyl 4-benzyl (2R)-2-{2- [(7-fluoro-2,2-dimethyl-2,3-dihydro- 1-benzofuran-5-yl)oxy]ethyl} piperazine-1,4-dicarboxylate | 529 |

In the same manner as in Reference Example 341, the following compounds (Reference Examples 720-734) shown in Table 10-1-Table 10-2 were obtained.

TABLE 10-1

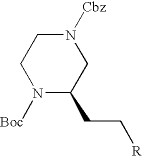

| Ref. Ex. No. | R | Compound | MS(ESI+) |
|---|---|---|---|
| 720 | 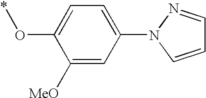 | 1-tert-Butyl 4-benzyl (2R)-2-{2-(2- methoxy-4-(1H-pyrazol-1-yl)phenoxy] ethyl}piperazine-1,4-dicarboxylate | 537 |
| 721 | 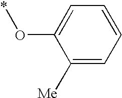 | 1-tert-Butyl 4-benzyl (2R)-2-[2-(2- methylphenoxy)ethyl] piperazine-1,4-dicarboxylate | 455 |
| 722 | 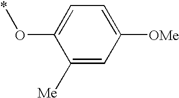 | 1-tert-Butyl 4-benzyl (2R)-2-[2-(4- methoxy-2-methylphenoxy)ethyl] piperazine-1,4-dicarboxylate | 485 |
| 723 | 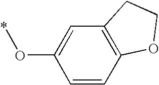 | 1-tert-Butyl 4-benzyl (2R)-2-[2- (2,3-dihydro-1-benzofuran-5-yloxy) ethyl]piperazine-1,4-dicarboxylate | 483 |
| 724 | 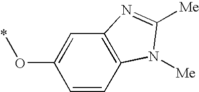 | 1-tert-Butyl 4-benzyl (2R)-2-{2-[(1,2- dimethyl-1H-benzimidazol-5-yl)oxy] ethyl}piperazine-1,4-dicarboxylate | 509 |
| 725 | 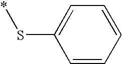 | 1-tert-Butyl 4-benzyl (2R)-2-[2- (phenylthio)ethyl]piperazine-1,4- dicarboxylate | 457 |
| 726 | 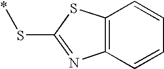 | 1-tert-Butyl 4-benzyl (2R)-2-[2- (1,3-benzothiazol-2-ylthio)ethyl] piperazine-1,4-dicarboxylate | 514 |
| 727 | 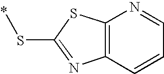 | 1-tert-Butyl 4-benzyl (2R)-2-[2-([1,3] thiazolo[5,4-b]pyridin-2-ylthio) ethyl]piperazine-1,4-dicarboxylate | 515 |
| 728 | 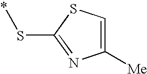 | 1-tert-Butyl 4-benzyl (2R)-2-{2- [(4-methyl-1,3-thiazol-2-yl)thio] ethyl}piperazine-1,4-dicarboxylate | 478 |
| 729 | 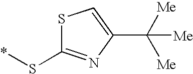 | 1-tert-Butyl 4-benzyl (2R)-2-{2- [4-tert-butyl-1,3-thiazol-2-yl)thio] ethyl}piperazine-1,4-dicarboxylate | 520 |

TABLE 10-2

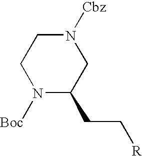

| Ref. Ex. No. | R | Compound | MS(ESI+) |
|---|---|---|---|
| 730 | 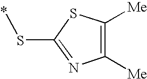 | 1-tert-Butyl 4-benzyl (2R)-2-{2-[(4,5- dimethyl-1,3-thiazol-2-yl)thio]ethyl} piperazine-1,4-dicarboxylate | 492 |
| 731 | 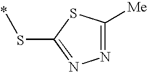 | 1-tert-Butyl 4-benzyl (2R)-2-{2-[(5- methyl-1,3,4-thiadiazol-2-yl)thio] ethyl}piperazine-1,4-dicarboxylate | 479 |
| 732 | 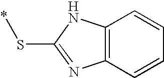 | 1-tert-Butyl 4-benzyl (2R)-2-[2-(1H- benzimidazol-2-ylthio)ethyl] piperazine-1,4-dicarboxylate | 497 |
| 733 | 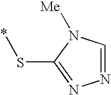 | 1-tert-Butyl 4-benzyl (2R)-2-{2-[(4- methyl-4H-1,2,4-triazol-3-yl)thio] ethyl}piperazine-1,4-dicarboxylate | 462 |
| 734 | 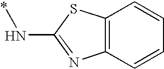 | 1-tert-Butyl 4-benzyl (2R)-2-[2-(1,3- benzothiazol-2-ylamino)ethyl] piperazine-1,4-dicarboxylate | 497 |

### Reference Example 735

### 1-tert-Butyl 4-benzyl (2R)-2-[2-(phenylsulfinyl)ethyl]piperazine-1,4-dicarboxylate

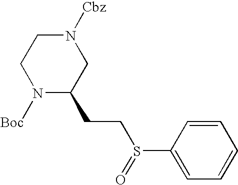

1-tert-Butyl 4-benzyl (2R)-2-[2-(phenylthio)ethyl]piperazine-1,4-dicarboxylate (0.8 g) was dissolved in dichloromethane (10 ml), and the solution was ice-cooled. mCPBA (0.3 g) was added thereto, and the mixture was stirred at 0° C. for 1 hr. mCPBA (0.2 g) was further added under ice-cooling, and the mixture was stirred at 0° C. for 3 hr. The reaction mixture was poured into aqueous sodium hydrogensulfite solution, and the mixture was extracted with ethyl acetate. The extract was washed with saturated brine, and dried over anhydrous sodium sulfate, and the solvent was evaporated under reduced pressure. The residue was subjected to silica gel column chromatography, and the fraction eluted with ethyl acetate-hexane (1:4) was concentrated under reduced pressure to give the object compound (0.78 g) as an amorphous solid.

MS (ESI+, m/e) 473 (M+1)

### Reference Example 736

### 1-tert-Butyl 4-benzyl (2R)-2-[2-(phenylsulfonyl)ethyl]piperazine-1,4-dicarboxylate

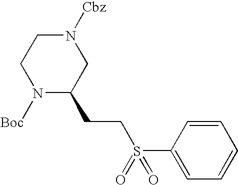

1-tert-Butyl 4-benzyl (2R)-2-[2-(phenylthio)ethyl]piperazine-1,4-dicarboxylate (0.8 g) was dissolved in dichloromethane (10 ml), and the solution was ice-cooled. mCPBA (0.6 g) was added thereto, and the mixture was stirred at room temperature for 1 hr. The reaction mixture was poured into aqueous sodium hydrogensulfite solution, and the mixture was extracted with ethyl acetate. The extract was washed with saturated brine, and dried over anhydrous sodium sulfate, and the solvent was evaporated under reduced pressure. The residue was subjected to silica gel column chromatography, and the fraction eluted with ethyl acetate-hexane (1:4) was concentrated under reduced pressure to give the object compound (0.85 g) as an amorphous solid.

MS (ESI+, m/e) 489 (M+1)

In the same manner as in Reference Example 663, the following compounds (Reference Examples 737-756) shown in Table 11-1-Table 11-2 were obtained.

TABLE 11-1

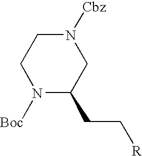

| Ref. Ex. No. | R | Compound | MS(ESI+) |
|---|---|---|---|
| 737 | 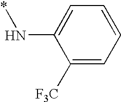 | 1-tert-Butyl 4-benzyl (2R)-2-(2{[2- (trifluoromethyl)phenyl]amino} ethyl)piperazine-1,4-dicarboxylate | 508 |
| 738 | 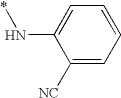 | 1-tert-Butyl 4-benzyl (2R)-2-{2-[(2- cyanophenyl)amino]ethyl} piperazine-1,4-dicarboxylate | 465 |
| 739 | 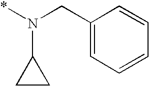 | 1-tert-Butyl 4-benzyl (2R)-2-{2- [benzyl(cyclopropyl)amino]ethyl} piperazine-1,4-dicarboxylate | 494 |
| 740 | 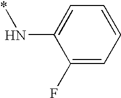 | 1-tert-Butyl 4-benzyl (2R)-2-{2-[(2- fluorophenyl)amino]ethyl) piperazine-1,4-dicarboxylate | 458 |
| 741 | 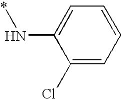 | 1-tert-Butyl 4-benzyl (2R)-2-{2-[(2- chlorophenyl)amino}ethyl} piperazine-1,4-dicarboxylate | 474 |
| 742 | 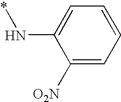 | 1-tert-Butyl 4-benzyl (2R)-2-{2-[(2- nitrophenyl)amino]ethyl} piperazine-1,4-dicarboxylate | 485 |
| 743 | 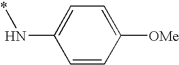 | 1-tert-Butyl 4-benzyl (2R)-2-{2-[(4- methoxyphenyl)amino]ethyl} piperazine-1,4-dicarboxylate | 470 |
| 744 | 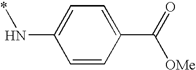 | 1-tert-Butyl 4-benzyl (2R)-2-(2-{[4- (methoxycarbonyl)phenyl]amino} ethyl)piperazine-1,4-dicarboxylate | 498 |
| 745 | 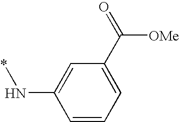 | 1-tert-Butyl 4-benzyl (2R)-2-(2-{[3- (methoxycarbonyl)phenyl]amino} ethyl)piperazine-1,4-dicarboxylate | 498 |
| 746 | 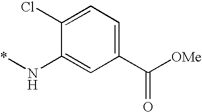 | 1-tert-Butyl 4-benzyl (2R)-2-(2-{[2- chloro-5-(methoxycarbonyl)phenyl] amino}ethyl)piperazine- 1,4-dicarboxylate | 532 |

TABLE 11-2

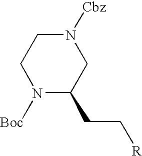

| Ref. Ex. No. | R | Compound | MS(ESI+) |
|---|---|---|---|
| 747 | 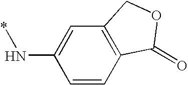 | 1-tert-Butyl 4-benzyl (2R)-2-{2- [(1-oxo-1,3-dihydro-2-benzofuran-5- yl)amino]ethyl}piperazine-1,4- dicarboxylate | 496 |
| 748 | 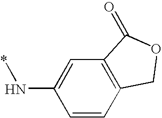 | 1-tert-Butyl 4-benzyl (2R)-2-{2- [(3-oxo-1,3-dihydro-2-benzofuran-5- yl)amino]ethyl}piperazine-1,4- dicarboxylate | 496 |
| 749 | 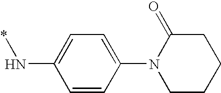 | 1-tert-Butyl 4-benzyl (2R)-2-(2-{[4- (2-oxopiperidin-1-yl)phenyl]amino} ethyl)piperazine-1,4-dicarboxylate | 537 |
| 750 | 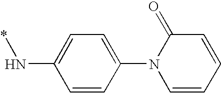 | 1-tert-Butyl 4-benzyl (2R)-2-(2-{[4- (2-oxopyridin-1(2H)-yl)phenyl] amino}ethyl)piperazine- 1,4-dicarboxylate | 533 |
| 751 | 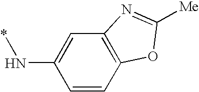 | 1-tert-Butyl 4-benzyl (2R)-2-{2-[(2- methyl-1,3-benzoxazol-5-yl)amino] ethyl}piperazine-1,4-dicarboxylate | 495 |
| 752 | 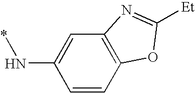 | 1-tert-Butyl 4-benzyl (2R)-2-{2-[(2- ethyl-1,3-benzoxazol-5-yl)amino] ethyl}piperazine-1,4-dicarboxylate | 509 |
| 753 | 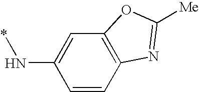 | 1-tert-Butyl 4-benzyl (2R)-2-{2-[(2- methyl-1,3-benzoxazol-6-yl)amino] ethyl}piperazine-1,4-dicarboxylate | 495 |
| 754 | 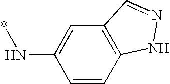 | 1-tert-Butyl 4-benzyl (2R)-2-[2- (1H-indazol-5-ylamino)ethyl] piperazine-1,4-dicarboxylate | 480 |
| 755 | 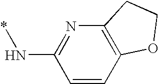 | 1-tert-Butyl 4-benzyl (2R)-2-[2-(2,3- dihydrofuro[3,2-b]pyridin-5- ylamino)ethyl]piperazine-1,4- dicarboxylate | 483 |
| 756 | 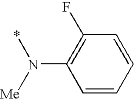 | 1-tert-Butyl 4-benzyl (2R)-2-{2- [(2-fluorophenyl)(methyl)amino] ethyl}piperazine-1,4-dicarboxylate | 472 |

In the same manner as in Reference Example 383 or Reference Example 665, the following compounds (Reference Examples 757-783) shown in Table 12-1-Table 12-3 were obtained. In the column of “Acid” in the Tables, the compounds described as “TFA” were synthesized according to the method of Reference Example 383 and the compounds described as “HCl” were synthesized according to the method of Reference Example 665.

TABLE 12-1

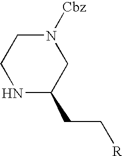

| Ref. Ex. No. | R | Compound | Acid | MS(ESI+) |
|---|---|---|---|---|
| 757 | 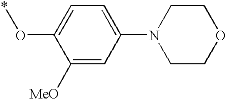 | Benzyl (3R)-3-[2-(2-methoxy-4- morpholinophenoxy)ethyl] piperazine-1-carboxylate | TFA | 456 |
| 758 | 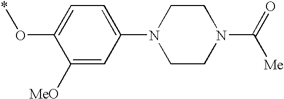 | Benzyl (3R)-3-{2-[4-(4-acetyl- piperazin-1-yl)-2-methoxyphenoxy] ethyl}piperazine-1-carboxylate | TFA | 497 |
| 759 | 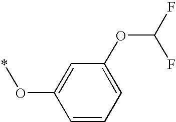 | Benzyl (3R)-3-{2-[3- (difluoromethoxy)phenoxy]ethyl} piperazine-1-carboxylate | TFA | 407 |
| 760 | 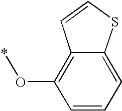 | Benzyl (3R)-3-[2-(1-benzothien-4- yloxy)ethyl]piperazine-1-carboxylate | HCl | 397 |
| 761 | 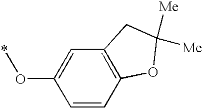 | Benzyl (3R)-3-{2-[(2,2-dimethyl- 2,3-dihydro-1-benzofuran-5-yl) oxy]ethyl}piperazine-1-carboxylate | HCl | 411 |
| 762 | 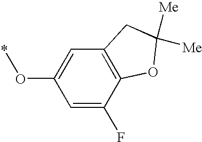 | Benzyl (3R)-3-{2-[(7-fluoro-2,2- dimethyl-2,3-dihydro-1-benzofuran- 5-yl)oxy]ethyl}piperazine-1- carboxylate | HCl | 429 |
| 763 | 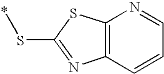 | Benzyl (3R)-3-[2-([1,3]thia- zolo[5,4-b]pyridin-2-ylthio)ethyl] piperazine-1-carboxylate | HCl | 415 |
| 764 | 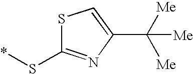 | Benzyl (3R)-3-{2-[(4-tert-butyl- 1,3-thiazol-2-yl)thio]ethyl} piperazine-1-carboxylate | HCl | 420 |
| 765 | 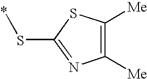 | Benzyl (3R)-3-{2-[(4,5-dimethyl- 1,3-thiazol-2-yl)thio]ethyl} piperazine-1-carboxylate | HCl | 392 |

TABLE 12-2

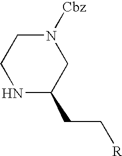

| Ref. Ex. No. | R | Compound | Acid | MS(ESI+) |
|---|---|---|---|---|
| 766 | 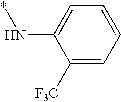 | Benzyl (3R)-3-(2-{[2- (trifluoromethyl)phenyl]amino} ethyl)piperazine-1-carboxylate | HCl | 408 |
| 767 | 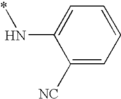 | Benzyl (3R)-3-{2-[(2- cyanophenyl)amino]ethyl} piperazine-1-carboxylate | HCl | 365 |
| 768 | 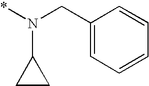 | Benzyl (3R)-3-{2- [benzyl(cyclopropyl)amino] ethyl}piperazine-1-carboxylate | HCl | 394 |
| 769 | 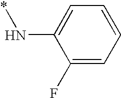 | Benzyl (3R)-3-{2-[(2- fluorophenyl)amino]ethyl} piperazine-1-carboxylate | HCl | 358 |
| 770 | 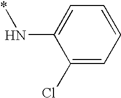 | Benzyl (3R)-3-{2-[(2- chlorophenyl)amino]ethyl} piperazine-1-carboxylate | HCl | 374 |
| 771 | 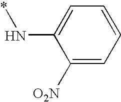 | Benzyl (3R)-3-{2-[(2- nitrophenyl)amino]ethyl} piperazine-1-carboxylate | HCl | 385 |
| 772 | 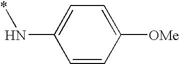 | Benzyl (3R)-3-{2-[(4- methoxyphenyl)amino]ethyl} piperazine-1-carboxylate | HCl | 370 |
| 773 | 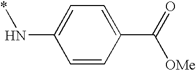 | Benzyl (3R)-3-(2-{[4- (methoxycarbonyl)phenyl]amino} ethyl)piperazine-1-carboxylate | HCl | 398 |
| 774 | 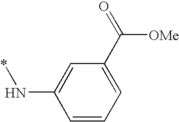 | Benzyl (3R)-3-(2-{[3- (methoxycarbonyl)phenyl]amino} ethyl)piperazine-1-carboxylate | HCl | 398 |
| 775 | 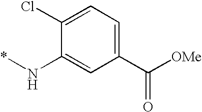 | Benzyl (3R)-3-(2-{[2-chloro-5- (methoxycarbonyl)phenyl]amino} ethyl)piperazine-1-carboxylate | HCl | 432 |

TABLE 12-3

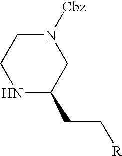

| Ref. Ex. No. | R | Compound | Acid | MS(ESI+) |
|---|---|---|---|---|
| 776 | 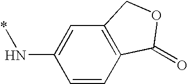 | Benzyl (3R)-3-{2-[(1-oxo-1,3- dihydro-2-benzofuran-5-yl)amino] ethyl}piperazine-1-carboxylate | HCl | 396 |
| 777 | 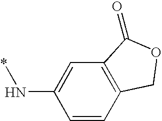 | Benzyl (3R)-3-{2-[(3-oxo-1,3- dihydro-2-benzofuran-5-yl)amino] ethyl}piperazine-1-carboxylate | HCl | 396 |
| 778 | 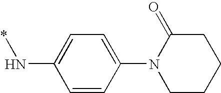 | Benzyl (3R)-3-(2-{[4-(2-oxo- piperidin-1-yl)phenyl]amino} ethyl)piperazine-1-carboxylate | HCl | 437 |
| 779 | 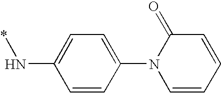 | Benzyl (3R)-3-(2-{[4-(2-oxo- pyridin-1(2H)-yl)phenyl]amino} ethyl)piperazine-1-carboxylate | HCl | 433 |
| 780 | 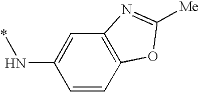 | Benzyl (3R)-3-{2-[(2-methyl-1,3- benzoxazol-5-yl)amino]ethyl} piperazine-1-carboxylate | HCl | 395 |
| 781 | 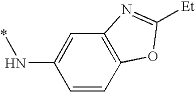 | Benzyl (3R)-3-{2-[(2-ethyl-1,3- benzoxazol-5-yl)amino]ethyl} piperazine-1-carboxylate | HCl | 409 |
| 782 | 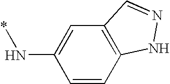 | Benzyl (3R)-3-[2-(1H-indazol-5- ylamino)ethyl]piperazine-1- carboxylate | HCl | 380 |
| 783 | 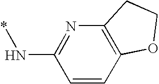 | Benzyl (3R)-3-[2-(2,3-dihydro- furo[3,2-b]pyridin-5-ylamino) ethyl]piperazine-1-carboxylate | HCl | 383 |

In the same manner as in Reference Example 425, the following compounds (Reference Examples 784-803) shown in Table 13-1-Table 13-2 were obtained.

TABLE 13-1

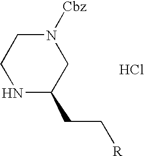

| Ref. Ex. No. | R | Compound | MS(ESI+) |
|---|---|---|---|
| 784 | 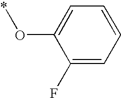 | Benzyl (3R)-3-[2-(2-fluoro- phenoxy)ethyl]piperazine-1- carboxylate hydrochloride | 359 |
| 785 | 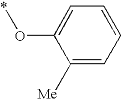 | Benzyl (3R)-3-[2-(2-methyl- phenoxy)ethyl]piperazine-1- carboxylate hydrochloride | 355 |
| 786 | 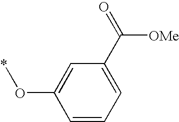 | Benzyl (3R)-3-{2-[3- (methoxycarbonyl)phenoxy]ethyl} piperazine-1-carboxylate hydrochloride | 399 |
| 787 | 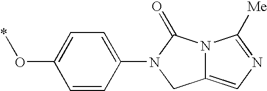 | Benzyl (3R)-3-{2-[4-(5-methyl- 3-oxo-1H-imidazo[1,5-c]imidazol- 2(3H)-yl)phenoxy]ethyl}piperazine-1- carboxylate hydrochloride | 476 |
| 788 | 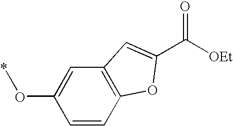 | Benzyl (3R)-3-(2-{[2-(ethoxycarbonyl)- 1-benzofuran-5-yl]oxy}ethyl) piperazine-1-carboxylate hydrochloride | 453 |
| 789 | 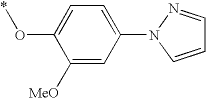 | Benzyl (3R)-3-{2-[2-methoxy-4-(1H- pyrazol-1-yl)phenoxy]ethyl}piperazine- 1-carboxylate hydrochloride | 437 |
| 790 | 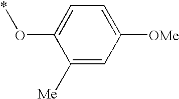 | Benzyl (3R)-3-[2-(4-methoxy-2- methylphenoxy)ethyl]piperazine-1- carboxylate hydrochloride | 385 |
| 791 | 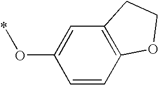 | Benzyl (3R)-3-[2-(2,3-dihydro-1- benzofuran-5-yloxy)ethyl}piperazine- 1-carboxylate hydrochloride | 383 |
| 792 | 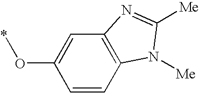 | Benzyl (3R)-3-{2-[(1,2-dimethyl-1H- benzimidazol-5-yl)oxy]ethyl} piperazine-1-carboxylate hydrochloride | 409 |
| 793 | 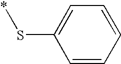 | Benzyl (3R)-3-[2-(phenylthio]ethyl] piperazinel-1-carboxylate hydrochloride | 357 |

TABLE 13-2

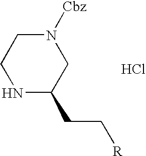

| Ref. Ex. No. | R | Compound | MS(ESI+) |
|---|---|---|---|
| 794 | 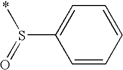 | Benzyl (3R)-3-[2-(phenylsulfinyl) ethyl]piperazine-1- carboxylate hydrochloride | 373 |
| 795 | 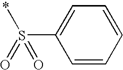 | Benzyl (3R)-3-[2-(phenylsulfonyl) ethyl]piperazine-1- carboxylate hydrochloride | 389 |
| 796 | 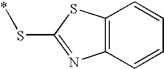 | Benzyl (3R)-3-[2-(1,3-benzothiazol- 2-ylthio)ethyl]piperazine-1- carboxylate hydrochloride | 414 |
| 797 | 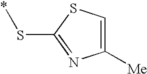 | Benzyl (3R)-3-{2-[(4-methyl-1,3- thiazol-2-yl)thio]ethyl}piperazine- 1-carboxylate hydrochloride | 378 |
| 798 | 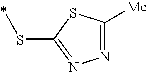 | Benzyl (3R)-3-{2-[(5-methyl-1,3,4- thiadiazol-2-yl)thio]ethyl}piperazine- 1-carboxylate hydrochloride | 379 |
| 799 | 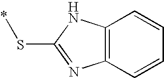 | Benzyl (3R)-3-[2-(1H-benzimidazol-2- ylthio)ethyl]piperazine-1- carboxylate hydrochloride | 397 |
| 800 | 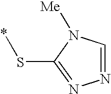 | Benzyl (3R)-3-{2-[(4-methyl- 4H-1,2,4-triazol-3-yl)thio]ethyl} piperazine-1-carboxylate hydrochloride | 362 |
| 801 | 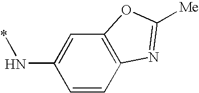 | Benzyl (3R)-3-{2-[(2-methyl-1,3- benzoxazol-6-yl)amino]ethyl} piperazine-1-carboxylate dihydrochloride | 395 |
| 802 | 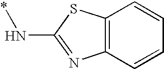 | Benzyl (3R)-3-[2-(1,3-benzothiazol- 2-ylamino)ethyl]piperazine-1- carboxylate dihydrochloride | 397 |
| 803 | 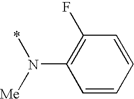 | Benzyl (3R)-3-{2-[(2-fluorophenyl) (methyl)amino]ethyl}piperazine-1- carboxylate dihydrochloride | 372 |

### Reference Example 804

### Benzyl (3R)-3-{2-[(4-acetylphenyl)amino]ethyl}piperazine-1-carboxylate

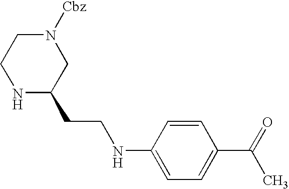

4-Acetylaniline (540 mg) was dissolved in DMF (20 ml), and the solution was ice-cooled. Sodium hydride (60% in oil, 160 mg) was added thereto, and the mixture was stirred at 0° C. for 15 min. After stirring, 1-tert-butyl 4-benzyl (2R)-2-{2-[(methylsulfonyl)oxy]ethyl}piperazine-1,4-dicarboxylate (885 mg) was added thereto, and the mixture was stirred at room temperature for 1 hr. The reaction mixture was poured into ice-cooled saturated aqueous sodium hydrogen carbonate, and the mixture was extracted with ethyl acetate. The extract was dried over anhydrous sodium sulfate, and the solvent was evaporated under reduced pressure. The residue was subjected to silica gel column chromatography, and the fraction eluted with ethyl acetate-hexane (1:1) was concentrated under reduced pressure to give 1-tert-butyl 4-benzyl (2R)-2-{2-[(4-acetylphenyl)amino]ethyl}piperazine-1,4-dicarboxylate (740 mg) as an oil. The total amount thereof was dissolved in ethyl acetate (5 ml), 4N hydrogen chloride-ethyl acetate solution (1 ml) was added thereto, and the mixture was stirred at room temperature for 1 hr. The reaction mixture was concentrated under reduced pressure, the residue was suspended in ethyl acetate, and the mixture was neutralized with saturated aqueous sodium hydrogen carbonate. The organic layer was dried over anhydrous sodium sulfate, and the solvent was evaporated under reduced pressure to give the object compound (650 mg).

MS (ESI+, m/e) 382 (M+1)

### Reference Example 805

### [(2R)-4-Benzyl-3,6-dioxopiperazin-2-yl]acetic acid

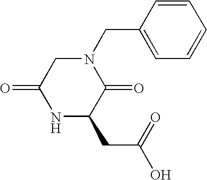

Benzyl [(2R)-4-benzyl-3,6-dioxopiperazin-2-yl]acetate (2.00 g) was dissolved in methanol (40 ml), 20% palladium hydroxide-carbon (50% containing water, 500 mg) was added thereto, and the mixture was subjected to catalytic reduction at ambient temperature and normal pressure for 17 hr. The catalyst was filtered off, and the filtrate was concentrated under reduced pressure. The crystals were collected by filtration to give the object compound (1.41 g).

¹H-NMR (DMSO-d₆) δ 2.68 (1H, dd), 2.85 (1H, dd), 3.78 (2H, q), 4.24 (1H, s), 4.36 (1H, d), 4.69 (1H, d), 7.27-7.36 (5H, m), 8.22 (1H, s), 12.50 (1H, br s)

MS (ESI+, m/e) 263 (M+1)

### Reference Example 806

### 2-[(2R)-4-Benzyl-3,6-dioxopiperazin-2-yl]-N-phenylacetamide

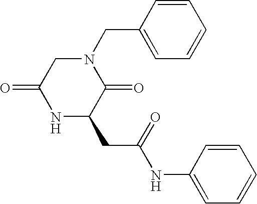

[(2R)-4-Benzyl-3,6-dioxopiperazin-2-yl]acetic acid (262 mg), aniline (102 mg) and HATU (570 mg) were dissolved in pyridine (5 ml), and the mixture was stirred at room temperature for 1 hr. To the reaction mixture was added 1N hydrochloric acid (40 ml), and the precipitated crystals were collected by filtration, washed with water, and vacuum-dried to give the object compound (320 mg).

MS (ESI+, m/e) 338 (M+1)

In the same manner as in Reference Example 806, the following compounds (Reference Examples 807-826) shown in Table 14-1-Table 14-2 were obtained.

TABLE 14-1

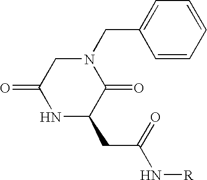

| Ref. Ex. No. | R | Compound | MS(ESI+) |
|---|---|---|---|
| 807 | 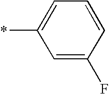 | 2-[(2R)-4-Benzyl-3,6-dioxopiperazin- 2-yl]-N-(3-fluorophenyl)acetamide | 356 |
| 808 | 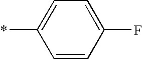 | 2-[(2R)-4-Benzyl-3,6-dioxopiperazin- 2-yl]-N-(4-fluorophenyl)acetamide | 356 |
| 809 | 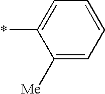 | 2-[(2R)-4-Benzyl-3,6-dioxopiperazin- 2-yl]-N-(2-methylphenyl)acetamide | 352 |
| 810 | 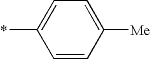 | 2-[(2R)-4-Benzyl-3,6-dioxopiperazin- 2-yl]-N-(4-methylphenyl)acetamide | 352 |
| 811 | 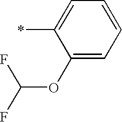 | 2-[(2R)-4-Benzyl-3,6-dioxopiperazin- 2-yl]-N-[2-(difluoromethoxy)phenyl] acetamide | 404 |
| 812 | 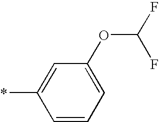 | 2-[(2R)-4-Benzyl-3,6-dioxopiperazin- 2-yl]-N-[3-(difluoromethoxy)phenyl] acetamide | 404 |
| 813 | 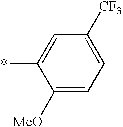 | 2-[(2R)-4-Benzyl-3,6-dioxopiperazin- 2-yl]-N-[2-methoxy-5-(trifluoromethyl) phenyl]acetamide | 436 |
| 814 | 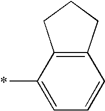 | 2-[(2R)-4-Benzyl-3,6-dioxopiperazin- 2-yl]-N-(2,3-dihydro-1H-inden-4- yl)acetamide | 378 |
| 815 | 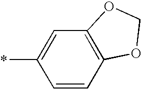 | N-(1,3-Benzodioxol-5-yl)-2-[(2R)-4- benzyl-3,6-dioxopiperazin-2- yl]acetamide | 382 |
| 816 | 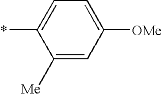 | 2-[(2R)-4-Benzyl-3,6-dioxopiperazin- 2-yl]-N-(4-methoxy-2- methylphenyl)acetamide | 382 |

TABLE 14-2

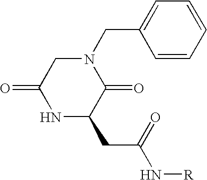

| Ref. Ex. No. | R | Compound | MS(ESI+) |
|---|---|---|---|
| 817 | 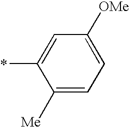 | 2-[(2R)-4-Benzyl-3,6-dioxopiperazin- 2-yl]-N-(5-methoxy-2-methylphenyl)acetamide | 382 |
| 818 | 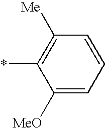 | 2-[(2R)-4-Benzyl-3,6-dioxopiperazin- 2-yl]-N-(2-methoxy-6-methylphenyl)acetamide | 382 |
| 819 | 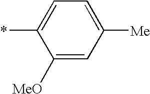 | 2-[(2R)-4-Benzyl-3,6-dioxopiperazin- 2-yl]-N-(2-methoxy-4-methylphenyl)acetamide | 382 |
| 820 | 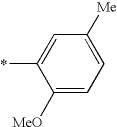 | 2-[(2R)-4-Benzyl-3,6-dioxopiperazin- 2-yl]-N-(2-methoxy-5-methylphenyl)acetamide | 382 |
| 821 | 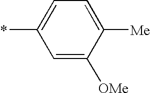 | 2-[(2R)-4-Benzyl-3,6-dioxopiperazin- 2-yl]-N-(3-methoxy-4-methylphenyl)acetamide | 382 |
| 822 | 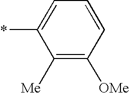 | 2-[(2R)-4-Benzyl-3,6-dioxopiperazin- 2-yl]-N-(3-methoxy-2-methylphenyl)acetamide | 382 |
| 823 | 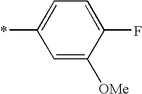 | 2-[(2R)-4-Benzyl-3,6-dioxopiperazin- 2-yl]-N-(4-fluoro-3-methoxyphenyl)acetamide | 386 |
| 824 | 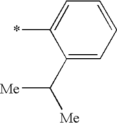 | 2-[(2R)-4-Benzyl-3,6-dioxopiperazin- 2-yl]-N-(2-isopropylphenyl)acetamide | 380 |
| 825 | 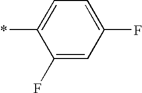 | 2-[(2R)-4-Benzyl-3,6-dioxopiperazin- 2-yl]-N-(2,4-difluorophenyl)acetamide | 374 |
| 826 | 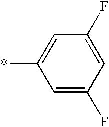 | 2-[(2R)-4-Benzyl-3,6-dioxopiperazin- 2-yl]-N-(3,5-difluorophenyl)acetamide | 374 |

### Reference Example 827

### N-{2-[(2R)-4-Benzylpiperazin-2-yl]ethyl}aniline

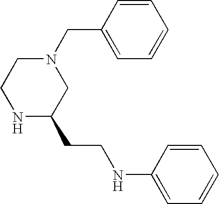

A mixture of 2-[(2R)-4-benzyl-3,6-dioxopiperazin-2-yl]-N-phenylacetamide (320 mg) and THF (10 ml) was ice-cooled, and lithium aluminum hydride (216 mg) was added by small portions. The mixture was stirred at room temperature for 30 min, and then at 60° C. for 15 hr, and cooled to −78° C. Ethanol-ethyl acetate (1:1, 1 ml) and 1N aqueous sodium hydroxide solution (2 ml) were successively added dropwise. After the completion of the dropwise addition, the mixture was stirred at room temperature for 40 min. The insoluble material was filtered, and washed with ethyl acetate. The filtrate was washed with saturated brine, and dried over anhydrous magnesium sulfate, and the solvent was evaporated under reduced pressure. The residue was subjected to basic silica gel column chromatography, and the fraction eluted with ethyl acetate was concentrated under reduced pressure to give the object compound (145 mg) as an oil.

MS (ESI+, m/e) 296 (M+1)

In the same manner as in Reference Example 827, the following compounds (Reference Examples 828-847) shown in Table 15-1-Table 15-2 were obtained.

TABLE 15-1

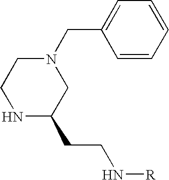

| Ref. Ex. No. | R | Compound | MS(ESI+) |
|---|---|---|---|
| 828 | 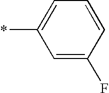 | N-{2-[(2R)-4-Benzylpiperazin-2- yl]ethyl}-3-fluoroaniline | 314 |
| 829 | 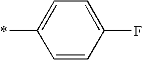 | N-{2-[(2R)-4-Benzylpiperazin-2- yl]ethyl}-4-fluoroaniline | 314 |
| 830 | 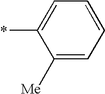 | N-{2-[(2R)-4-Benzylpiperazin-2- yl]ethyl}-2-methylaniline | 310 |
| 831 | 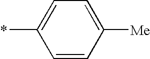 | N-{2-[(2R)-4-Benzylpiperazin-2- yl]ethyl}-4-methylaniline | 310 |
| 832 | 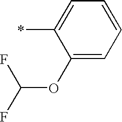 | N-{2-[(2R)-4-Benzylpiperazin-2- yl]ethyl}-2-(difluoromethoxy)aniline | 362 |
| 833 | 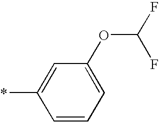 | N-{2-[(2R)-4-Benzylpiperazin-2- yl]ethyl}-3-(difluoromethoxy)aniline | 362 |
| 834 | 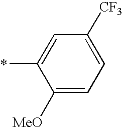 | N-{2-[(2R)-4-Benzylpiperazin-2- yl]ethyl}-2-methoxy-5- (trifluoromethyl)aniline | 394 |
| 835 | 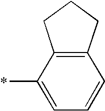 | N-{2-[(2R)-4-Benzylpiperazin-2- yl]ethyl}indan-4-amine | 336 |
| 836 | 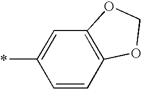 | N-{2-[(2R)-4-Benzylpiperazin-2- yl]ethyl}-1,3-benzodioxol-5-amine | 340 |
| 837 | 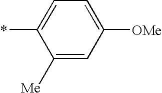 | N-{2-[(2R)-4-Benzylpiperazin-2- yl]ethyl}-4-methoxy-2-methylaniline | 340 |

TABLE 15-2

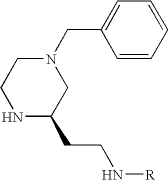

| Ref. Ex. No. | R | Compound | MS(ESI+) |
|---|---|---|---|
| 838 | 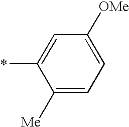 | N-{2-[(2R)-4-Benzylpiperazin-2-yl] ethyl}-5-methoxy-2-methylaniline | 340 |
| 839 | 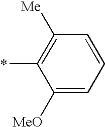 | N-{2-[(2R)-4-Benzylpiperazin-2-yl] ethyl}-2-methoxy-6-methylaniline | 340 |
| 840 | 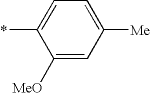 | N-{2-[(2R)-4-Benzylpiperazin-2-yl] ethyl}-2-methoxy-4-methylaniline | 340 |
| 841 | 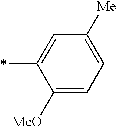 | N-{2-[(2R)-4-Benzylpiperazin-2-yl] ethyl}-2-methoxy-5-methylaniline | 340 |
| 842 | 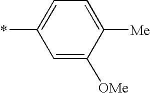 | N-{2-[(2R)-4-Benzylpiperazin-2-yl] ethyl}-3-methoxy-4-methylaniline | 340 |
| 843 | 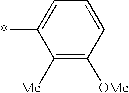 | N-{2-[(2R)-4-Benzylpiperazin-2-yl] ethyl}-3-methoxy-2-methylaniline | 340 |
| 844 | 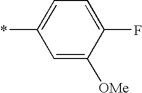 | N-{2-[(2R)-4-Benzylpiperazin-2-yl] ethyl}-4-fluoro-3-methoxyaniline | 344 |
| 845 | 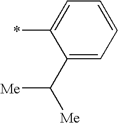 | N-{2-[(2R)-4-Benzylpiperazin-2-yl] ethyl}-2-isopropylaniline | 338 |
| 846 | 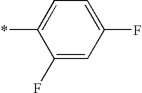 | N-{2-[(2R)-4-Benzylpiperazin-2-yl] ethyl}2,4-difluoroaniline | 332 |
| 847 | 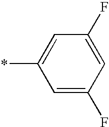 | N-{2-[(2R)-4-Benzylpiperazin-2-yl] ethyl}-3,5-difluoroaniline | 332 |

### Reference Example 848

### N-{2-[(2R)-4-Benzylpiperazin-2-yl]ethyl}-4-(difluoromethoxy)aniline

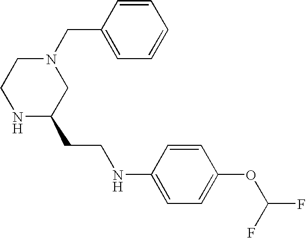

[(2R)-4-Benzyl-3,6-dioxopiperazin-2-yl]acetic acid (262 mg) and 4-(difluoromethoxy)aniline (159 mg) were dissolved in pyridine (5 ml), HATU (570 mg) was added, and the mixture was stirred at room temperature for 2 hr. The reaction mixture was concentrated under reduced pressure, the residue was diluted with heptane, and the mixture was again concentrated under reduced pressure to remove pyridine. The residue was dissolved in ethyl acetate, and the solution was washed successively with saturated aqueous sodium hydrogen carbonate, 1N hydrochloric acid and saturated brine, and dried over anhydrous sodium sulfate, and the solvent was evaporated under reduced pressure. The crystals were collected by filtration to give 2-[(2R)-4-benzyl-3,6-dioxopiperazin-2-yl]-N-[4-(difluoromethoxy)phenyl]acetamide (370 mg). The total amount thereof was suspended in THF (10 ml), and the suspension was ice-cooled. Lithium aluminum hydride (200 mg) was added by small portions, and the mixture was stirred at room temperature for 30 min, and then at 60° C. for 3 hr, and was cooled to −78° C. Water (0.2 ml), 4N aqueous sodium hydroxide solution (0.2 ml) and water (0.6 ml) were successively added dropwise. After the completion of the dropwise addition, the mixture was stirred at room temperature for 40 min. The insoluble material was filtered, and washed with THF. The filtrate was washed with saturated brine, and dried over anhydrous magnesium sulfate, and the solvent was evaporated under reduced pressure. The residue was subjected to silica gel column chromatography, and the fraction eluted with ethyl acetate-methanol (1:0-10:1) was concentrated under reduced pressure to give the object compound (330 mg) as an oil.

MS (ESI+, m/e) 362 (M+1)

In the same manner as in Reference Example 848, the following compounds (Reference Examples 849-854) shown in Table 16 were obtained.

TABLE 16

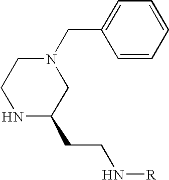

| Ref. Ex. No. | R | Compound | MS(ESI+) |
|---|---|---|---|
| 849 | 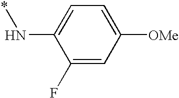 | N-{2-[(2R)-4-Benzylpiperazin-2- yl]ethyl}-2-fluoro-4-methoxyaniline | 344 |
| 850 | 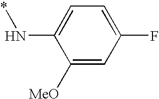 | N-{2-[(2R)-4-Benzylpiperazin-2- yl]ethyl}-4-fluoro-2-methoxyaniline | 344 |
| 851 | 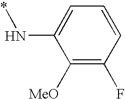 | N-{2-[(2R)-4-Benzylpiperazin-2- yl]ethyl}-3-fluoro-2-methoxyaniline | 344 |
| 852 | 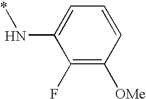 | N-{2-[(2R)-4-Benzylpiperazin-2- yl]ethyl}-2-fluoro-3-methoxyaniline | 344 |
| 853 | 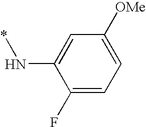 | N-{2-[(2R)-4-Benzylpiperazin-2- yl]ethyl}-2-fluoro-5-methoxyaniline | 344 |
| 854 | 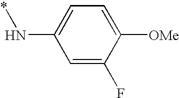 | N-{2-[(2R)-4-Benzylpiperazin-2- yl]ethyl}-2-fluoro-3-methoxyaniline | 344 |

### Reference Example 855

### 2-[(2R)-4-Benzylpiperazin-2-yl]-N-phenylacetamide

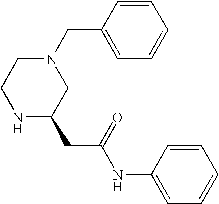

[(2R)-4-Benzyl-1-(tert-butoxycarbonyl)piperazin-2-yl]acetic acid (200 mg) and aniline (55 mg) were dissolved in pyridine (5 ml), HATU (340 mg) was added, and the mixture was stirred at room temperature for 2 hr. The reaction mixture was concentrated under reduced pressure, the residue was diluted with heptane, and the mixture was again concentrated under reduced pressure to remove pyridine. The residue was subjected to silica gel column chromatography, and the fraction eluted with ethyl acetate-hexane (1:1-1:0) was concentrated under reduced pressure to give tert-butyl (2R)-2-(2-anilino-2-oxoethyl)-4-benzylpiperazine-1-carboxylate (120 mg) as an amorphous solid. This was dissolved in ethyl acetate (2 ml), 4N hydrogen chloride-ethyl acetate solution (5 ml) was added, and the mixture was stirred at room temperature for 1 hr. The reaction mixture was concentrated under reduced pressure, the residue was suspended in ethyl acetate, and the suspension was neutralized with saturated aqueous sodium hydrogen carbonate. The organic layer was dried over anhydrous sodium sulfate, and the solvent was evaporated under reduced pressure to give the object compound (110 mg).

MS (ESI+, m/e) 310 (M+1)

In the same manner as in Reference Example 855, the following compound (Reference Example 856) was obtained.

### Reference Example 856

### 2-[(2R)-4-Benzylpiperazin-2-yl]-N-methyl-N-phenylacetamide

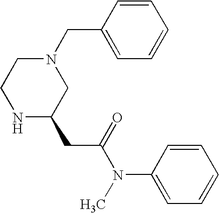

MS (ESI+, m/e) 324 (M+1)

### Reference Example 857

### N-(3-Methoxyphenyl)-2-nitrobenzenesulfonamide

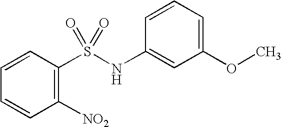

3-Methoxyaniline (1.2 g) and triethylamine (2 ml) were dissolved in THF (20 ml), and the solution was ice-cooled. 2-Nitrobenzenesulfonyl chloride (2.65 g) was added thereto, and the mixture was stirred at 0° C. for 1 hr. The reaction mixture was poured into ice water, and the mixture was extracted with ethyl acetate. The extract was dried over anhydrous sodium sulfate, and the solvent was evaporated under reduced pressure to give the object compound (3.2 g).

¹H-NMR (CDCl₃) δ 3.77 (3H, s), 6.68-6.78 (2H, m), 6.82 (1H, t), 7.16 (1H, t), 7.55-7.76 (2H, m), 7.83-7.91 (2H, m)

In the same manner as in Reference Example 857, the following compounds (Reference Examples 858-862) were obtained.

### Reference Example 858

### N-(3-Acetylphenyl)-2-nitrobenzenesulfonamide

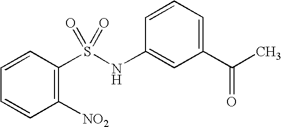

¹H-NMR (CDCl₃) δ 2.57 (3H, s), 7.36-7.54 (3H, m), 7.54-7.66 (1H, m), 7.68-7.79 (3H, m), 7.83-7.90 (2H, m)

### Reference Example 859

### 2-Nitro-N-[4-(trifluoromethoxy)phenyl]benzenesulfonamide

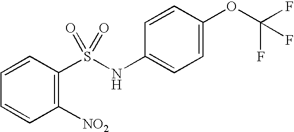

¹H-NMR (CDCl₃) δ 7.05-7.39 (4H, m), 7.53-8.00 (4H, m)

### Reference Example 860

### 2-Nitro-N-[4-(1H-pyrazol-1-yl)phenyl]benzenesulfonamide

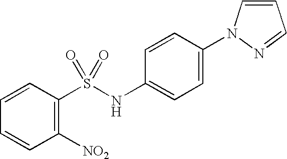

¹H-NMR (DMSO-d₆) δ 6.47-6.58 (1H, m), 7.23 (2H, d), 7.67-8.09 (5H, m), 8.39 (1H, d), 10.81 (1H, s)

### Reference Example 861

### N-(2-Methyl-1,3-benzothiazol-5-yl)-2-nitrobenzenesulfonamide

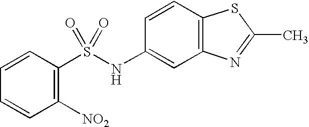

¹H-NMR (DMSO-d₆) δ 2.75 (3H, s), 3.58 (1H, br s), 7.18 (1H, dd), 7.60 (1H, d), 7.73-8.04 (5H, m), 10.88 (4H, s)

### Reference Example 862

### N-(2-Methyl-1,3-benzothiazol-6-yl)-2-nitrobenzenesulfonamide

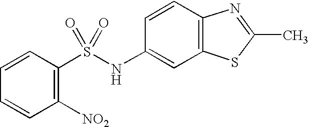

¹H-NMR (CDCl₃) δ 2.82 (3H, s), 7.20 (1H, dd), 7.46 (1H, br s), 7.50-7.58 (1H, m), 7.64-7.73 (1H, m), 7.75-7.83 (3H, m), 7.87 (1H, dd)

### Reference Example 863

### 1-tert-Butyl 4-benzyl (2R)-2-(2-{(3-acetylphenyl)[(2-nitrophenyl)sulfonyl]amino}ethyl)piperazine-1,4-dicarboxylate

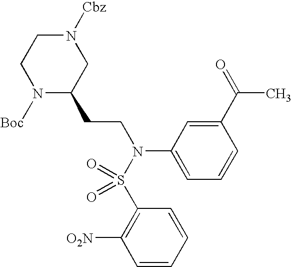

1-tert-Butyl 4-benzyl (2R)-2-{2-[(methylsulfonyl)oxy]ethyl}piperazine-1,4-dicarboxylate (885 mg) was dissolved in DMF (20 ml), N-(3-acetylphenyl)-2-nitrobenzenesulfonamide (1.3 g) and cesium carbonate (1.3 g) were added thereto. The mixture was stirred at 60° C. for 12 hr, and the reaction solution was partitioned between ethyl acetate and water. The organic layer was washed with saturated brine, and dried over anhydrous sodium sulfate, and the solvent was evaporated under reduced pressure. The residue was subjected to silica gel column chromatography, and the fraction eluted with ethyl acetate-hexane (1:1) was concentrated under reduced pressure to give the object compound (700 mg) as an amorphous solid.

MS (ESI+, m/e) 555 (M+1)

### Reference Example 864

### 1-tert-Butyl 4-benzyl (2R)-2-(2-{[(2-nitrophenyl)sulfonyl][4-(trifluoromethoxy)phenyl]amino}ethyl)piperazine-1,4-dicarboxylate

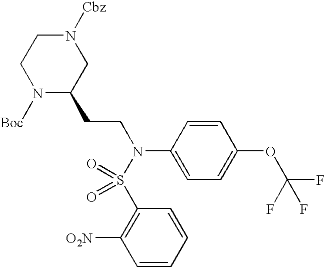

2-Nitro-N-[4-(trifluoromethoxy)phenyl]benzenesulfonamide (652 mg), 1-tert-butyl 4-benzyl (2R)-2-(2-hydroxyethyl)piperazine-1,4-dicarboxylate (550 mg) and triphenylphosphine (472 mg) were dissolved in toluene (20 ml), DEAD (40% toluene solution, 1 ml) was added, and the mixture was stirred at room temperature for 12 hr. The reaction mixture was concentrated under reduced pressure. The residue was subjected to silica gel column chromatography, and the fraction eluted with ethyl acetate-hexane (1:1) was concentrated under reduced pressure to give the object compound (740 mg) as an amorphous solid.

MS (ESI+, m/e) 709 (M+1)

In the same manner as in Reference Example 864, the following compounds (Reference Examples 865-867) were obtained.

### Reference Example 865

### 1-tert-Butyl 4-benzyl (2R)-2-(2-{[(2-nitrophenyl)sulfonyl][4-(1H-pyrazol-1-yl)phenyl]amino}ethyl)piperazine-1,4-dicarboxylate

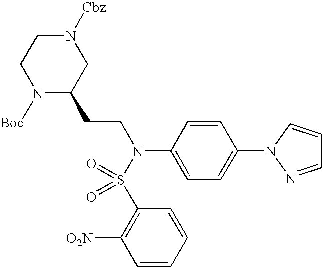

MS (ESI+, m/e) 691 (M+1)

### Reference Example 866

### 1-tert-Butyl 4-benzyl (2R)-2-(2-{(2-methyl-1,3-benzothiazol-5-yl)[(2-nitrophenyl)sulfonyl]amino}ethyl)piperazine-1,4-dicarboxylate

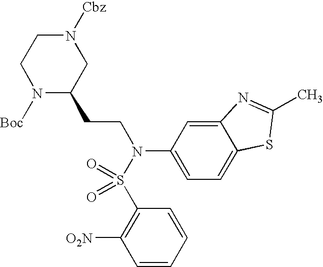

MS (ESI+, m/e) 696 (M+1)

### Reference Example 867

### 1-tert-Butyl 4-benzyl (2R)-2-(2-{(2-methyl-1,3-benzothiazol-6-yl)[(2-nitrophenyl)sulfonyl]amino}ethyl)piperazine-1,4-dicarboxylate

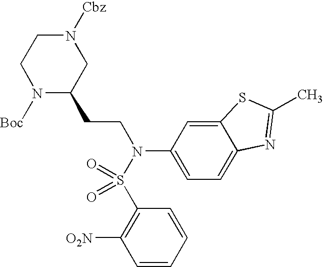

MS (ESI+, m/e) 696 (M+1)

### Reference Example 868

### Benzyl (3R)-3-{2-[(3-acetylphenyl)amino]ethyl}piperazine-1-carboxylate

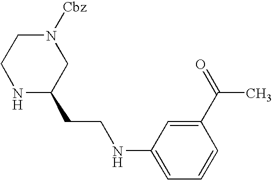

1-tert-Butyl 4-benzyl (2R)-2-(2-{(3-acetylphenyl)[(2-nitrophenyl)sulfonyl]amino}ethyl)piperazine-1,4-dicarboxylate (700 mg) and mercaptoacetic acid (0.22 ml) were dissolved in DMF (5 ml), lithium hydroxide monohydrate (264 mg) was added, and the mixture was stirred at room temperature for 3 hr. The reaction mixture was diluted with ethyl acetate, and poured into saturated aqueous sodium hydrogen carbonate. The organic layer was dried over anhydrous sodium sulfate, and the solvent was evaporated under reduced pressure. The residue was subjected to silica gel column chromatography, and the fraction eluted with ethyl acetate-methanol (1:1) was concentrated under reduced pressure to give 1-tert-butyl 4-benzyl (2R)-2-{2-[(3-acetylphenyl)amino]ethyl}piperazine-1,4-dicarboxylate (190 mg) as an amorphous solid. The total amount thereof was dissolved in ethyl acetate (5 ml), 4N hydrogen chloride-ethyl acetate solution (10 ml) was added, and the mixture was stirred at room temperature for 1 hr. The reaction mixture was concentrated under reduced pressure, the residue was suspended in ethyl acetate, and the suspension was neutralized with saturated aqueous sodium hydrogen carbonate. The organic layer was dried over anhydrous sodium sulfate, and the solvent was evaporated under reduced pressure to give the object compound (120 mg).

MS (ESI+, m/e) 382 (M+1)

In the same manner as in Reference Example 868, the following compounds (Reference Examples 869-870) were obtained.

### Reference Example 869

### Benzyl (3R)-3-(2-{[4-(trifluoromethoxy)phenyl]amino}ethyl)piperazine-1-carboxylate

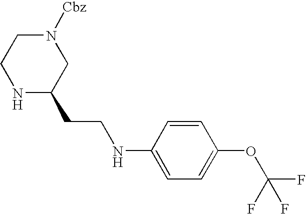

MS (ESI+, m/e) 424 (M+1)

### Reference Example 870

### Benzyl (3R)-3-(2-{[4-(1H-pyrazol-1-yl)phenyl]amino}ethyl)piperazine-1-carboxylate

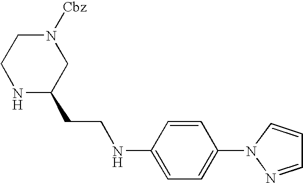

MS (ESI+, m/e) 406 (M+1)

### Reference Example 871

### Benzyl (3R)-3-(2-{(2-methoxyphenyl)[(2-nitrophenyl)sulfonyl]amino}ethyl)piperazine-1-carboxylate

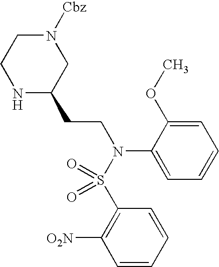

A mixture of 1-tert-butyl 4-benzyl (2R)-2-{2-[(methylsulfonyl)oxy]ethyl}piperazine-1,4-dicarboxylate (530 mg), N-(2-methoxyphenyl)-2-nitrobenzenesulfonamide (490 mg), potassium carbonate (415 mg) and DMF (10 ml) was stirred at 50° C. for 12 hr, and poured into water, and the mixture was extracted with ethyl acetate. The extract was washed with saturated brine, and dried over anhydrous sodium sulfate, and the solvent was evaporated under reduced pressure. The residue was subjected to silica gel column chromatography, and the fraction eluted with ethyl acetate-hexane (1:4-1:1) was concentrated under reduced pressure to give 1-tert-butyl 4-benzyl (2R)-2-(2-{(2-methoxyphenyl)[(2-nitrophenyl)sulfonyl]amino}ethyl)piperazine-1,4-dicarboxylate (650 mg) as an oil. This was dissolved in ethyl acetate (2 ml), 4N hydrogen chloride-ethyl acetate solution (1 ml) was added thereto, and the mixture was stirred at room temperature for 1 hr. The reaction mixture was concentrated under reduced pressure, the residue was suspended in ethyl acetate, and the suspension was neutralized with saturated aqueous sodium hydrogen carbonate. The organic layer was dried over anhydrous sodium sulfate, and the solvent was evaporated under reduced pressure to give the object compound (490 mg).

MS (ESI+, m/e) 555 (M+1)

In the same manner as in Reference Example 871, the following compound (Reference Example 872) was obtained.

### Reference Example 872

### Benzyl (3R)-3-(2-{(3-methoxyphenyl)[(2-nitrophenyl)sulfonyl]amino}ethyl)piperazine-1-carboxylate

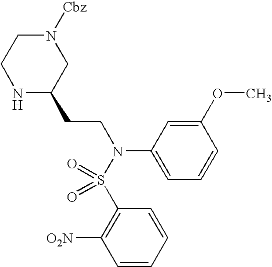

MS (ESI+, m/e) 555 (M+1)

### Reference Example 873

### Benzyl (3R)-3-(2-{(2-methyl-1,3-benzothiazol-5-yl)[(2-nitrophenyl)sulfonyl]amino}ethyl)piperazine-1-carboxylate

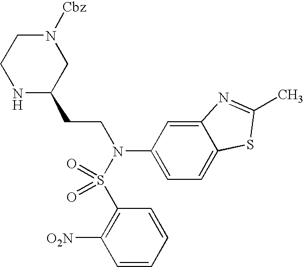

1-tert-Butyl 4-benzyl (2R)-2-(2-{(2-methyl-1,3-benzothiazol-5-yl)[(2-nitrophenyl)sulfonyl]amino}ethyl)piperazine-1,4-dicarboxylate (420 mg) was dissolved in ethyl acetate (5 ml), 4N hydrogen chloride-ethyl acetate solution (10 ml) was added, and the mixture was stirred at room temperature for 1 hr. The reaction mixture was concentrated under reduced pressure, the residue was suspended in ethyl acetate, and the suspension was neutralized with saturated aqueous sodium hydrogen carbonate. The organic layer was dried over anhydrous sodium sulfate, and the solvent was evaporated under reduced pressure to give the object compound (310 mg).

MS (ESI+, m/e) 596 (M+1)

In the same manner as in Reference Example 873, the following compound (Reference Example 874) was obtained.

### Reference Example 874

### Benzyl (3R)-3-(2-{(2-methyl-1,3-benzothiazol-6-yl)[(2-nitrophenyl)sulfonyl]amino}ethyl)piperazine-1-carboxylate

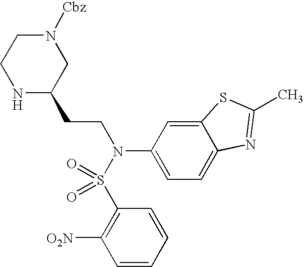

MS (ESI+, m/e) 596 (M+1)

In the same manner as in Reference Example 529, the following compounds (Reference Examples 875-877) were obtained.

### Reference Example 875

### 1-{[4-({(2R)-4-Benzyl-2-[(5-phenyl-1,3,4-oxadiazol-2-yl)methyl]piperazin-1-yl}carbonyl)-5-phenyl-1H-imidazol-1-yl]methyl}cyclohexanol

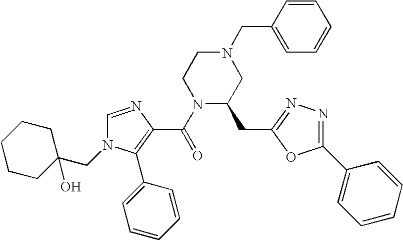

MS (ESI+, m/e) 617 (M+1)

### Reference Example 876

### 1-({4-[((2R)-4-Benzyl-2-{2-[(2-fluoro-4-methoxyphenyl)amino]ethyl}piperazin-1-yl)carbonyl]-5-phenyl-1H-imidazol-1-yl}methyl)cyclohexanol

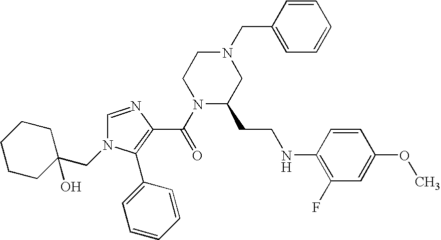

MS (ESI+, m/e) 626 (M+1)

### Reference Example 877

### 1-({4-[((2R)-4-Benzyl-2-{2-[(2-fluoro-3-methoxyphenyl)amino]ethyl}piperazin-1-yl)carbonyl]-5-phenyl-1H-imidazol-1-yl}methyl)cyclohexanol

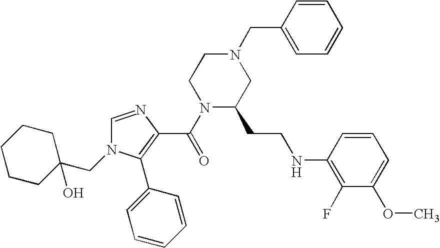

MS (ESI+, m/e) 626 (M+1)

### Reference Example 878

### Methyl [(1S,2S)-2-(4-{[(2R)-4-benzyl-2-(2-bromobenzyl)piperazin-1-yl]carbonyl}-5-phenyl-1H-imidazol-1-yl)cyclohexyl]carbamate

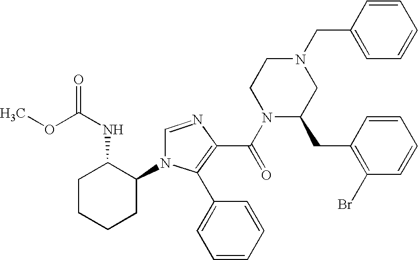

tert-Butyl (2R)-4-benzyl-2-(2-bromobenzyl)piperazine-1-carboxylate (1.78 g) was dissolved in methanol (5 ml), 4N hydrogen chloride-ethyl acetate solution (2 ml) was added, and the mixture was stirred at room temperature for 5 hr. The reaction mixture was concentrated under reduced pressure, and the residue was dissolved in DMF (30 ml). 1-{(1S,2S)-2-[(Methoxycarbonyl)amino]cyclohexyl}-5-phenyl-1H-imidazole-4-carboxylic acid (1.37 g), WSC.HCl (1.15 g), HOBt (757 mg) and N,N-diisopropylethylamine (3.56 ml) were added, and the mixture was stirred at 60° C. for 5 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to basic silica gel column chromatography, and the fraction eluted with ethyl acetate was concentrated under reduced pressure to give the object compound (2.31 g) as an amorphous solid.

MS (ESI+, m/e) 670 (M+1)

### Reference Example 879

### (1S,2R)-2-(4-{[(2S)-4-Benzyl-2-(hydroxymethyl)piperazin-1-yl]carbonyl}-5-phenyl-1H-imidazol-1-yl)-1-(methoxymethyl)cyclohexanol

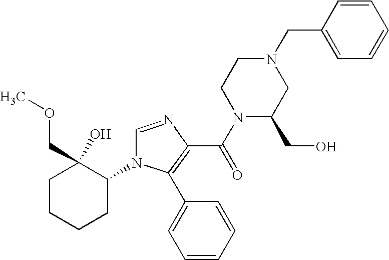

1-[(1R,2S)-2-Hydroxy-2-(methoxymethyl)cyclohexyl]-5-phenyl-1H-imidazole-4-carboxylic acid (330 mg) and [(2S)-4-benzylpiperazin-2-yl]methanol (206 mg) were suspended in DMF (10 ml), WSC.HCl (288 mg) and HOBt (189 mg) were added thereto, and the mixture was stirred at 60° C. for 5 hr. The reaction mixture was concentrated under reduced pressure, the residue was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate-methanol (4:1) was concentrated under reduced pressure to give the object compound (410 mg) as an amorphous solid.

MS (ESI+, m/e) 519 (M+1)

In the same manner as in Reference Example 879, the following compounds (Reference Examples 880-881) were obtained.

### Reference Example 880

### (1S,2R)-2-(4-{[(2R)-4-Benzyl-2-(2-bromobenzyl)piperazin-1-yl]carbonyl}-5-phenyl-1H-imidazol-1-yl)-1-(methoxymethyl)cyclohexanol

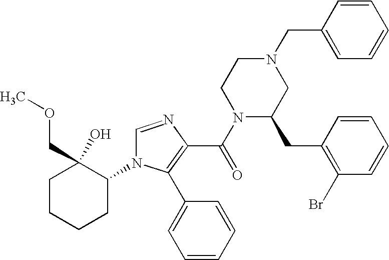

MS (ESI+, m/e) 657 (M+1)

### Reference Example 881

### tert-Butyl (3S)-4-({1-[(1R,2S)-2-hydroxy-2-(methoxymethyl)cyclohexyl]-5-phenyl-1H-imidazol-4-yl}carbonyl)-3-[(phenylthio)methyl]piperazine-1-carboxylate

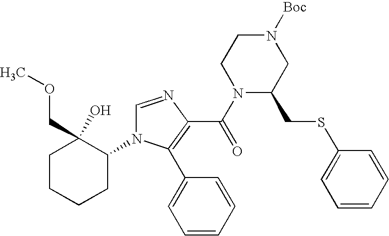

MS (ESI+, m/e) 621 (M+1)

### Reference Example 882

### tert-Butyl (3S)-4-({1-[(1R,2S)-2-hydroxy-2-(methoxymethyl)cyclohexyl]-5-phenyl-1H-imidazol-4-yl}carbonyl)-3-[(phenylsulfinyl)methyl]piperazine-1-carboxylate

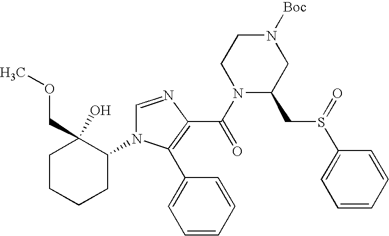

tert-Butyl (3S)-4-({1-[(1R,2S)-2-hydroxy-2-(methoxymethyl)cyclohexyl]-5-phenyl-1H-imidazol-4-yl}carbonyl)-3-[(phenylthio)methyl]piperazine-1-carboxylate (310 mg) was dissolved in dichloromethane (5 ml), and the solution was ice-cooled. mCPBA (123 mg) was added, and the mixture was stirred at 0° C. for 30 min. To the reaction mixture was added aqueous sodium thiosulfate solution, and the mixture was extracted with ethyl acetate. The extract was washed successively with saturated aqueous sodium hydrogen carbonate and saturated brine, and dried over anhydrous sodium sulfate, and the solvent was evaporated under reduced pressure. The residue was subjected to silica gel column chromatography, and the fraction eluted with ethyl acetate-methanol (4:1) was concentrated under reduced pressure to give the object compound (312 mg) as an amorphous solid.

MS (ESI+, m/e) 637 (M+1)

### Reference Example 883

### tert-Butyl (3S)-4-({1-[(1R,2S)-2-hydroxy-2-(methoxymethyl)cyclohexyl]-5-phenyl-1H-imidazol-4-yl}carbonyl)-3-[(phenylsulfonyl)methyl]piperazine-1-carboxylate

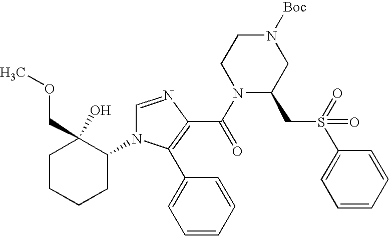

tert-Butyl (3S)-4-({1-[(1R,2S)-2-hydroxy-2-(methoxymethyl)cyclohexyl]-5-phenyl-1H-imidazol-4-yl}carbonyl)-3-[(phenylthio)methyl]piperazine-1-carboxylate (310 mg) was dissolved in dichloromethane (5 ml), and the solution was ice-cooled. mCPBA (247 mg) was added, and the mixture was stirred at 0° C. for 30 min. To the reaction mixture was added aqueous sodium thiosulfate solution, and the mixture was extracted with ethyl acetate. The extract was washed successively with saturated aqueous sodium hydrogen carbonate and saturated brine, and dried over anhydrous sodium sulfate, and the solvent was evaporated under reduced pressure. The residue was subjected to silica gel column chromatography, and the fraction eluted with ethyl acetate-methanol (4:1) was concentrated under reduced pressure to give the object compound (317 mg) as an amorphous solid.

MS (ESI+, m/e) 653 (M+1)

### Reference Example 884

### (1S,2R)-2-(4-{[(2R)-4-Benzyl-2-(biphenyl-2-ylmethyl)piperazin-1-yl]carbonyl}-5-phenyl-1H-imidazol-1-yl)-1-(methoxymethyl)cyclohexanol and (1S,2R)-2-(4-{[(2S)-4-benzyl-2-(biphenyl-2-ylmethyl)piperazin-1-yl]carbonyl}-5-phenyl-1H-imidazol-1-yl)-1-(methoxymethyl)cyclohexanol

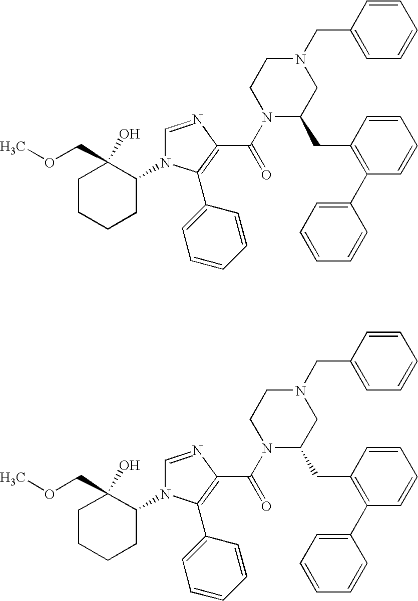

A solution of 1-[(1R,2S)-2-hydroxy-2-(methoxymethyl)cyclohexyl]-5-phenyl-1H-imidazole-4-carboxylic acid (200 mg), 1-benzyl-3-(biphenyl-2-ylmethyl)piperazine (240 mg), WSC.HCl (173 mg) and HOBt (110 mg) in DMF (7 ml) was stirred at room temperature for 15 hr, and poured into saturated aqueous sodium hydrogen carbonate, and the mixture was extracted with ethyl acetate. The extract was washed successively with water and saturated brine, and dried over anhydrous magnesium sulfate, and the solvent was evaporated under reduced pressure. The residue was subjected to silica gel column chromatography, and the fractions eluted with ethyl acetate-methanol (4:1) were concentrated under reduced pressure to give (1S,2R)-2-(4-{[(2R)-4-benzyl-2-(biphenyl-2-ylmethyl)piperazin-1-yl]carbonyl}-5-phenyl-1H-imidazol-1-yl)-1-(methoxymethyl)cyclohexanol (180 mg) as an amorphous solid, and (1S,2R)-2-(4-{[(2S)-4-benzyl-2-(biphenyl-2-ylmethyl)piperazin-1-yl]carbonyl}-5-phenyl-1H-imidazol-1-yl)-1-(methoxymethyl)cyclohexanol (130 mg) as an amorphous solid.

MS (ESI+, m/e) 655 (M+1)

MS (ESI+, m/e) 655 (M+1)

In the same manner as in Reference Example 884, the following compound (Reference Example 885) was obtained.

### Reference Example 885

### (1S,2R)-2-(4-{[(2R)-4-Benzyl-2-(2-methoxybenzyl)piperazin-1-yl]carbonyl}-5-phenyl-1H-imidazol-1-yl)-1-(methoxymethyl)cyclohexanol and (1S,2R)-2-(4-{[(2S)-4-benzyl-2-(2-methoxybenzyl)piperazin-1-yl]carbonyl}-5-phenyl-1H-imidazol-1-yl)-1-(methoxymethyl)cyclohexanol

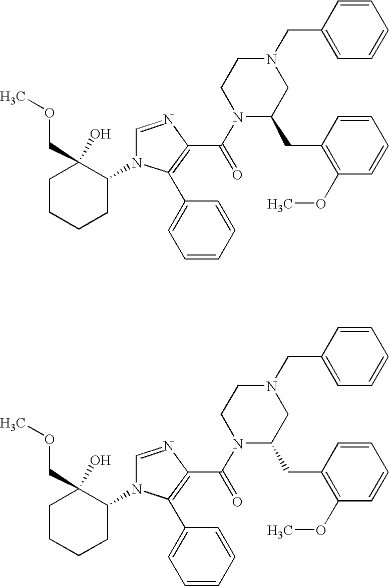

MS (ESI+, m/e) 609 (M+1)

MS (ESI+, m/e) 609 (M+1)

In the same manner as in Reference Example 519, the following compounds (Reference Examples 886-890) were obtained.

### Reference Example 886

### (1S,2R)-2-(4-{[(2R)-4-Benzyl-2-(2-morpholinobenzyl)piperazin-1-yl]carbonyl}-5-phenyl-1H-imidazol-1-yl)-1-(methoxymethyl)cyclohexanol

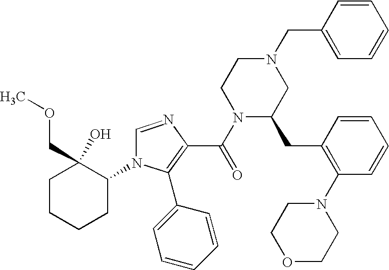

MS (ESI+, m/e) 664 (M+1)

### Reference Example 887

### (1S,2R)-2-[4-({(2R)-4-Benzyl-2-[2-(2-methoxypyridin-3-yl)benzyl]piperazin-1-yl}carbonyl)-5-phenyl-1H-imidazol-1-yl]-1-(methoxymethyl)cyclohexanol

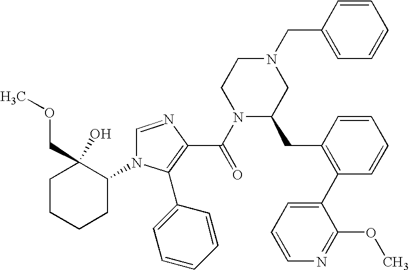

MS (ESI+, m/e) 686 (M+1)

### Reference Example 888

### (1S,2R)-2-(4-{[(2R)-4-Benzyl-2-(2-phenoxybenzyl)piperazin-1-yl]carbonyl}-5-phenyl-1H-imidazol-1-yl)-1-(methoxymethyl)cyclohexanol

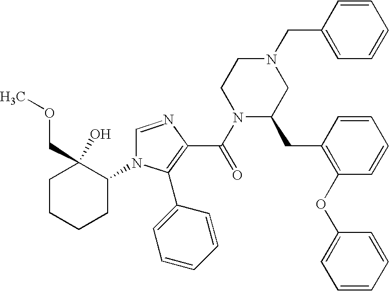

MS (ESI+, m/e) 671 (M+1)

### Reference Example 889

### (1S,2R)-2-[4-({(2R)-4-Benzyl-2-[2-(1-methyl-1H-pyrazol-5-yl)benzyl]piperazin-1-yl}carbonyl)-5-phenyl-1H-imidazol-1-yl]-1-(methoxymethyl)cyclohexanol

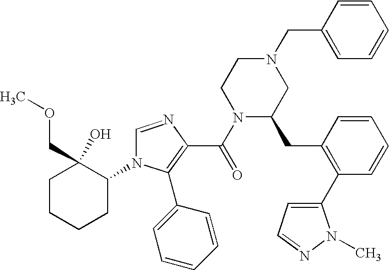

MS (ESI+, m/e) 659 (M+1)

### Reference Example 890

### Benzyl (3R)-4-({1-[(1R,2S)-2-hydroxy-2-(methoxymethyl)cyclohexyl]-5-phenyl-1H-imidazol-4-yl}carbonyl)-3-(2-{[3-(methoxycarbonyl)phenyl]amino}ethyl)piperazine-1-carboxylate

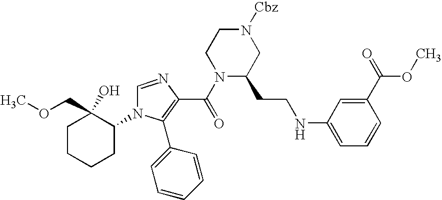

MS (ESI+, m/e) 710 (M+1)

### Reference Example 891

### Benzyl (3R)-4-({1-[(1R,2S)-2-hydroxy-2-(methoxymethyl)cyclohexyl]-5-phenyl-1H-imidazol-4-yl}carbonyl)-3-(2-{(2-methoxyphenyl)[(2-nitrophenyl)sulfonyl]amino}ethyl)piperazine-1-carboxylate

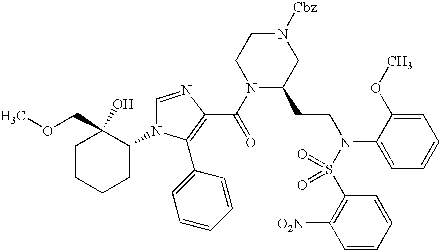

A solution of 1-[(1R,2S)-2-hydroxy-2-(methoxymethyl)cyclohexyl]-5-phenyl-1H-imidazole-4-carboxylic acid (490 mg), benzyl (3R)-3-(2-{(2-methoxyphenyl)[(2-nitrophenyl)sulfonyl]amino}ethyl)piperazine-1-carboxylate (290 mg), WSC.HCl (253 mg) and HOBt (175 mg) in DMF (10 ml) was stirred at room temperature for 12 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate was concentrated under reduced pressure to give the object compound (600 mg) as an amorphous solid.

MS (ESI+, m/e) 867 (M+1)

In the same manner as in Reference Example 891, the following compounds (Reference Examples 892-894) were obtained.

### Reference Example 892

### Benzyl (3R)-4-({1-[(1R,2S)-2-hydroxy-2-(methoxymethyl)cyclohexyl]-5-phenyl-1H-imidazol-4-yl}carbonyl)-3-(2-{(3-methoxyphenyl) [(2-nitrophenyl)sulfonyl]amino}ethyl)piperazine-1-carboxylate

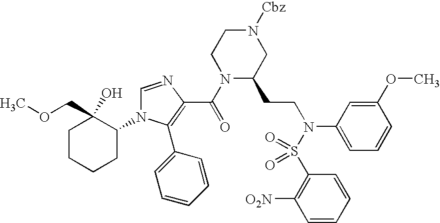

MS (ESI+, m/e) 867 (M+1)

### Reference Example 893

### Benzyl (3R)-4-({1-[(1R,2S)-2-hydroxy-2-(methoxymethyl)cyclohexyl]-5-phenyl-1H-imidazol-4-yl}carbonyl)-3-(2-{(2-methyl-1,3-benzothiazol-5-yl) [(2-nitrophenyl)sulfonyl]amino}ethyl)piperazine-1-carboxylate

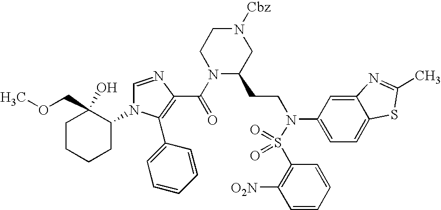

MS (ESI+, m/e) 908 (M+1)

### Reference Example 894

### Benzyl (3R)-4-({1-[(1R,2S)-2-hydroxy-2-(methoxymethyl)cyclohexyl]-5-phenyl-1H-imidazol-4-yl}carbonyl)-3-(2-{(2-methyl-1,3-benzothiazol-6-yl) [(2-nitrophenyl)sulfonyl]amino}ethyl)piperazine-1-carboxylate

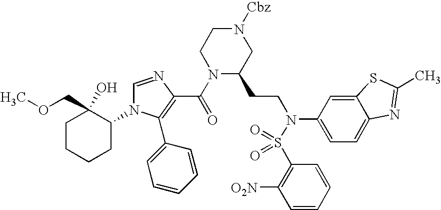

MS (ESI+, m/e) 908 (M+1)

### Reference Example 895

### Benzyl (3R)-4-({1-[(1R,2S)-2-hydroxy-2-(methoxymethyl)cyclohexyl]-5-phenyl-1H-imidazol-4-yl}carbonyl)-3-{2-[(2-methoxyphenyl)amino]ethyl}piperazine-1-carboxylate

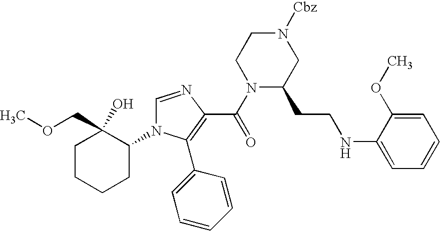

Benzyl (3R)-4-({1-[(1R,2S)-2-hydroxy-2-(methoxymethyl)cyclohexyl]-5-phenyl-1H-imidazol-4-yl}carbonyl)-3-(2-{(2-methoxyphenyl)[(2-nitrophenyl)sulfonyl]amino}ethyl)piperazine-1-carboxylate (300 mg) and mercaptoacetic acid (92 mg) were dissolved in DMF (5 ml), lithium hydroxide monohydrate (84 mg) was added, and the mixture was stirred at room temperature for 3 hr. The reaction mixture was diluted with ethyl acetate, and poured into saturated aqueous sodium hydrogen carbonate. The organic layer was dried over anhydrous sodium sulfate, and the solvent was evaporated under reduced pressure. The residue was subjected to silica gel column chromatography, and the fraction eluted with ethyl acetate-methanol (9:1) was concentrated under reduced pressure to give the object compound (190 mg) as an amorphous solid.

MS (ESI+, m/e) 682 (M+1)

In the same manner as in Reference Example 895, the following compounds (Reference Examples 896-898) were obtained.

### Reference Example 896

### Benzyl (3R)-4-({1-[(1R,2S)-2-hydroxy-2-(methoxymethyl)cyclohexyl]-5-phenyl-1H-imidazol-4-yl}carbonyl)-3-{2-[(3-methoxyphenyl)amino]ethyl}piperazine-1-carboxylate

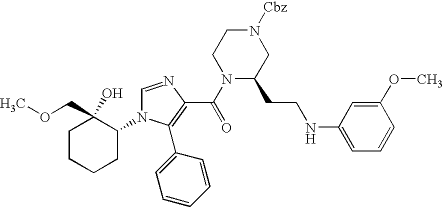

MS (ESI+, m/e) 682 (M+1)

### Reference Example 897

### Benzyl (3R)-4-({1-[(1R,2S)-2-hydroxy-2-(methoxymethyl)cyclohexyl]-5-phenyl-1H-imidazol-4-yl}carbonyl)-3-{2-[(2-methyl-1,3-benzothiazol-5-yl)amino]ethyl}piperazine-1-carboxylate

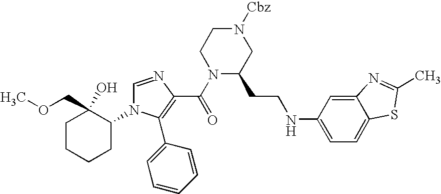

MS (ESI+, m/e) 723 (M+1)

### Reference Example 898

### Benzyl (3R)-4-({1-[(1R,2S)-2-hydroxy-2-(methoxymethyl)cyclohexyl]-5-phenyl-1H-imidazol-4-yl}carbonyl)-3-{2-[(2-methyl-1,3-benzothiazol-6-yl)amino]ethyl}piperazine-1-carboxylate

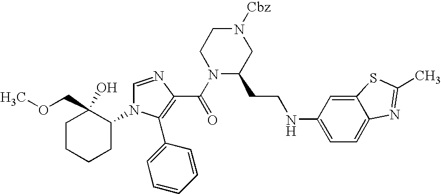

MS (ESI+, m/e) 723 (M+1)

In the same manner as in Reference Example 645, the following compounds (Reference Examples 899-901) were obtained.

### Reference Example 899

### tert-Butyl (3R)-4-({1-[(1R,2S)-2-hydroxy-2-(methoxymethyl)cyclohexyl]-5-phenyl-1H-imidazol-4-yl}carbonyl)-3-{2-[(piperidin-1-ylcarbonyl)oxy]ethyl}piperazine-1-carboxylate

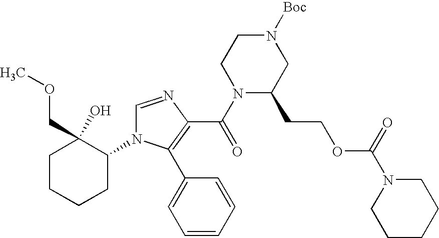

MS (ESI+, m/e) 654 (M+1)

### Reference Example 900

### tert-Butyl (3R)-3-{2-[(anilinocarbonyl)oxy]ethyl}-4-({1-[(1R,2S)-2-hydroxy-2-(methoxymethyl)cyclohexyl]-5-phenyl-1H-imidazol-4-yl}carbonyl)piperazine-1-carboxylate

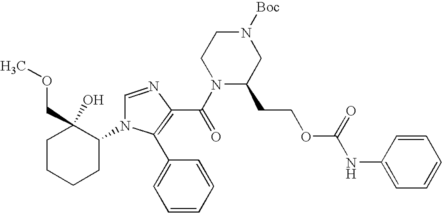

MS (ESI+, m/e) 662 (M+1)

### Reference Example 901

### [(2S)-4-benzyl-1-({1-[(1R,2S)-2-hydroxy-2-(methoxymethyl)cyclohexyl]-5-phenyl-1H-imidazol-4-yl}carbonyl)piperazin-2-yl]methyl phenylcarbamate

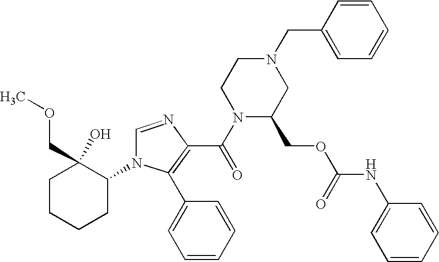

MS (ESI+, m/e) 638 (M+1)

### Reference Example 902

### Benzyl (3R)-3-{2-[acetyl(phenyl)amino]ethyl}-4-({1-[(1R,2S)-2-hydroxy-2-(methoxymethyl)cyclohexyl]-5-phenyl-1H-imidazol-4-yl}carbonyl)piperazine-1-carboxylate

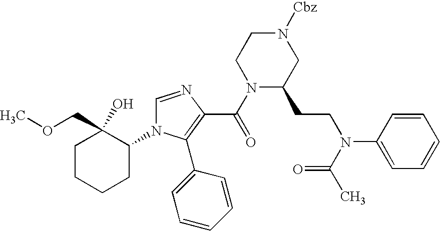

Benzyl (3R)-3-(2-anilinoethyl)-4-({1-[(1R,2S)-2-hydroxy-2-(methoxymethyl)cyclohexyl]-5-phenyl-1H-imidazol-4-yl}carbonyl)piperazine-1-carboxylate obtained in the course of the below-mentioned Example 428 (150 mg) and triethylamine (0.048 ml) were dissolved in dichloromethane (5 ml), and the solution was ice-cooled. Acetyl chloride (59 mg) was added, and the mixture was stirred at room temperature for 1 hr. The reaction mixture was poured into saturated aqueous sodium hydrogen carbonate, and the mixture was extracted with ethyl acetate. The extract was washed with saturated brine, and dried over anhydrous sodium sulfate, and the solvent was evaporated under reduced pressure. The residue was subjected to silica gel column chromatography, and the fraction eluted with ethyl acetate-methanol (1:0-9:2) was concentrated under reduced pressure to give the object compound (90 mg) as an amorphous solid.

MS (ESI+, m/e) 694 (M+1)

In the same manner as in Reference Example 902, the following compound (Reference Example 903) was obtained.

### Reference Example 903

### Benzyl (3R)-3-{2-[(cyclopropylcarbonyl)(phenyl)amino]ethyl}-4-({1-[(1R,2S)-2-hydroxy-2-(methoxymethyl)cyclohexyl]-5-phenyl-1H-imidazol-4-yl}carbonyl)piperazine-1-carboxylate

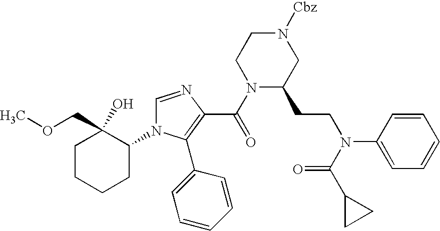

MS (ESI+, m/e) 720 (M+1)

### Reference Example 904

### tert-Butyl (3R)-3-[2-(4-bromo-2-fluorophenoxy)ethyl]-4-({1-[(1R,2S)-2-hydroxy-2-(methoxymethyl)cyclohexyl]-5-phenyl-1H-imidazol-4-yl}carbonyl)piperazine-1-carboxylate

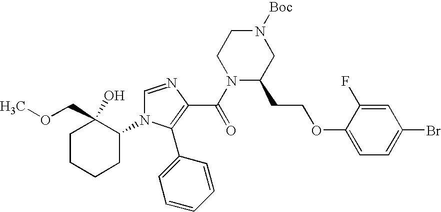

(1S,2R)-2-[4-({(2R)-2-[2-(4-Bromo-2-fluorophenoxy)ethyl]piperazin-1-yl}carbonyl)-5-phenyl-1H-imidazol-1-yl]-1-(methoxymethyl)cyclohexanol (the compound of the below-mentioned Example 257) (220 mg) was dissolved in THF (10 ml), di-tert-butyl bicarbonate (94 mg) was added, and the mixture was stirred at room temperature for 1 hr. The solvent was evaporated under reduced pressure. The residue was subjected to silica gel column chromatography, and the fraction eluted with ethyl acetate-methanol (9:1) was concentrated under reduced pressure to give the object compound (270 mg) as an amorphous solid.

MS (ESI+, m/e) 715 (M+1)

### Reference Example 905

### tert-Butyl (3R)-3-{2-[2-fluoro-4-(1H-pyrazol-1-yl)phenoxy]ethyl}-4-({1-[(1R,2S)-2-hydroxy-2-(methoxymethyl)cyclohexyl]-5-phenyl-1H-imidazol-4-yl}carbonyl)piperazine-1-carboxylate

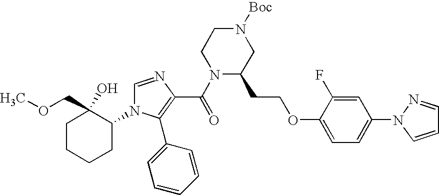

tert-Butyl (3R)-3-[2-(4-bromo-2-fluorophenoxy)ethyl]-4-({1-[(1R,2S)-2-hydroxy-2-(methoxymethyl)cyclohexyl]-5-phenyl-1H-imidazol-4-yl}carbonyl)piperazine-1-carboxylate (220 mg), potassium carbonate (85 mg), copper iodide (I) (19 mg) and pyrazole (42 mg) were suspended in DMF (5 ml), and the suspension was reacted at 160° C. for 5 min using microwave reactor. The reaction mixture was poured into water, and the mixture was extracted with ethyl acetate. The extract was washed with saturated brine, and dried over anhydrous sodium sulfate, and the solvent was evaporated under reduced pressure. The residue was subjected to basic silica gel column chromatography, and the fraction eluted with ethyl acetate-methanol (4:1) was concentrated under reduced pressure to give the object compound (30 mg) as an amorphous solid.

MS (ESI+, m/e) 703 (M+1)

### Reference Example 906

### tert-Butyl (3R)-3-[2-(1,3-dihydro-2H-isoindol-2-yl)ethyl]-4-({1-[(1R,2S)-2-hydroxy-2-(methoxymethyl)cyclohexyl]-5-phenyl-1H-imidazol-4-yl}carbonyl)piperazine-1-carboxylate

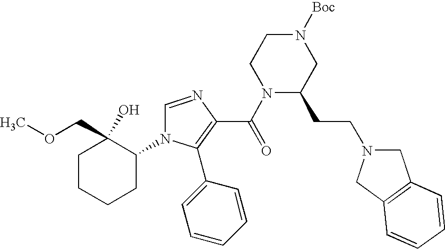

tert-Butyl (3R)-4-({1-[(1R,2S)-2-hydroxy-2-(methoxymethyl)cyclohexyl]-5-phenyl-1H-imidazol-4-yl}carbonyl)-3-(2-oxoethyl)piperazine-1-carboxylate (200 mg) and isoindoline (132 mg) were dissolved in dichloromethane-DMF (2:1, 3 ml), acetic acid (67 μl) was added, and the mixture was stirred for 30 min. Sodium triacetoxyborohydride (235 mg) was added thereto, and the mixture was stirred at room temperature for 12 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate-methanol (9:1) was concentrated under reduced pressure to give the object compound (110 mg) as an oil.

MS (ESI+, m/e) 644 (M+1)

In the same manner as in Reference Example 906, the following compounds (Reference Examples 907-909) were obtained.

### Reference Example 907

### tert-Butyl (3R)-3-[2-(3,4-dihydroisoquinolin-2(1H)-yl)ethyl]-4-({1-[(1R,2S)-2-hydroxy-2-(methoxymethyl)cyclohexyl]-5-phenyl-1H-imidazol-4-yl}carbonyl)piperazine-1-carboxylate

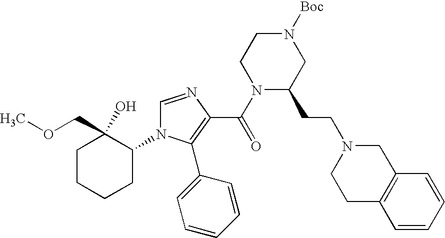

MS (ESI+, m/e) 658 (M+1)

### Reference Example 908

### tert-Butyl (3R)-3-[2-(3,4-dihydroquinolin-1(2H)-yl)ethyl]-4-({1-[(1R,2S)-2-hydroxy-2-(methoxymethyl)cyclohexyl]-5-phenyl-1H-imidazol-4-yl}carbonyl)piperazine-1-carboxylate

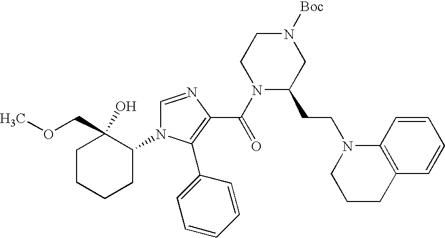

MS (ESI+, m/e) 658 (M+1)

### Reference Example 909

### tert-Butyl (3R)-4-({1-[(1R,2S)-2-hydroxy-2-(methoxymethyl)cyclohexyl]-5-phenyl-1H-imidazol-4-yl}carbonyl)-3-[2-(pyridin-2-ylamino)ethyl]piperazine-1-carboxylate

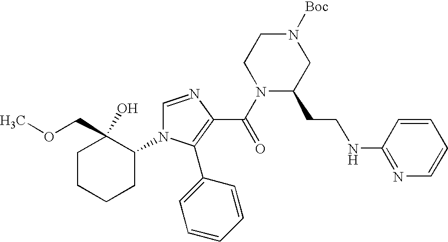

MS (ESI+, m/e) 619 (M+1)

In the same manner as in Reference Example 341, the following compounds (Reference Examples 910-912) were obtained.

### Reference Example 910

### 1-tert-Butyl 4-benzyl (2R)-2-[2-(2,3-dihydro-1-benzofuran-6-yloxy)ethyl]piperazine-1,4-dicarboxylate

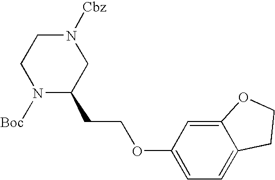

MS (ESI+, m/e) 483 (M+1)

### Reference Example 911

### 1-tert-Butyl 4-benzyl (2R)-2-{2-[(2-methyl-1,3-benzoxazol-5-yl)oxy]ethyl}piperazine-1,4-dicarboxylate

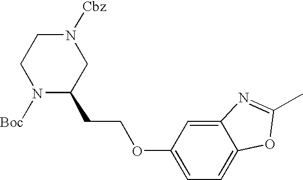

MS (ESI+, m/e) 496 (M+1)

### Reference Example 912

### 1-tert-Butyl 4-benzyl (2R)-2-[2-({4-[(acetyloxy)methyl]-1,3-thiazol-2-yl}thio)ethyl]piperazine-1,4-dicarboxylate

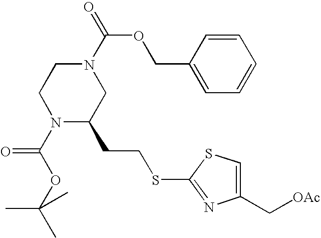

MS (ESI+, m/e) 492 (M+1)

In the same manner as in Reference Example 425, the following compounds (Reference Examples 913-915) were obtained.

### Reference Example 913

### Benzyl (3R)-3-[2-(2,3-dihydro-1-benzofuran-6-yloxy)ethyl]piperazine-1-carboxylate

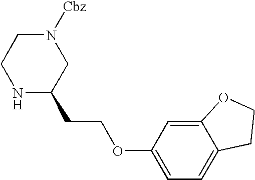

MS (ESI+, m/e) 383 (M+1)

### Reference Example 914

### Benzyl (3R)-3-{2-[(2-methyl-1,3-benzoxazol-5-yl)oxy]ethyl}piperazine-1-carboxylate

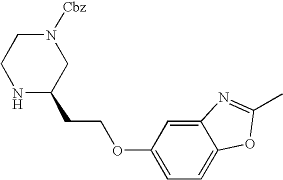

MS (ESI+, m/e) 396 (M+1)

### Reference Example 915

### Benzyl (3R)-3-[2-({4-[(acetyloxy)methyl]-1,3-thiazol-2-yl}thio)ethyl]piperazine-1-carboxylate

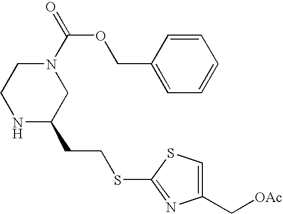

MS (ESI+, m/e) 392 (M+1)

In the same manner as in Reference Example 879, the following compound (Reference Example 916) was obtained.

### Reference Example 916

### tert-Butyl (3S)-4-({1-[(1R,2R)-2-(cyclopropylmethyl)-2-hydroxycyclohexyl]-5-phenyl-1H-imidazol-4-yl}carbonyl)-3-[(phenylthio)methyl]piperazine-1-carboxylate

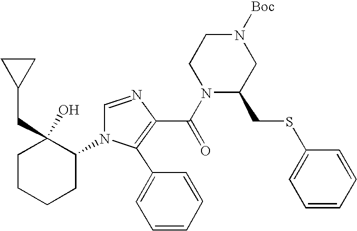

MS (ESI+, m/e) 631 (M+1)

In the same manner as in Reference Example 883, the following compound (Reference Example 917) was obtained.

### Reference Example 917

### tert-Butyl (3S)-4-({1-[(1R,2R)-2-(cyclopropylmethyl)-2-hydroxycyclohexyl]-5-phenyl-1H-imidazol-4-yl}carbonyl)-3-[(phenylsulfonyl)methyl]piperazine-1-carboxylate

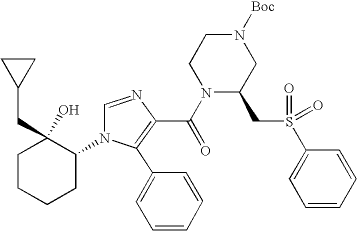

MS (ESI+, m/e) 663 (M+1)

### Reference Example 918

### 2-Ethyl-1,3-benzothiazol-5-ol and 2-isopropyl-1,3-benzothiazol-5-ol

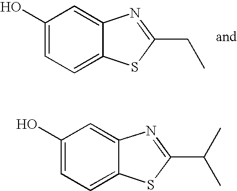

To an ice-cooled solution of diisopropylamine (1.5 ml) in THF (6 ml) was added dropwise n-butyllithium (5 ml, 2.5M hexane solution), and the mixture was stirred for 30 min. The mixture was added dropwise to a solution of 5-bromo-2-methyl-1,3-benzothiazole (1.14 g) in THF (6 ml) which was cooled to −78° C., and the mixture was stirred at the same temperature for 30 min. Methyl iodide (1.6 ml) was added, and the mixture was further stirred for 1 hr. To the reaction mixture was added ethyl acetate (50 ml), and the mixture was allowed to warm to room temperature, and washed successively with 1N hydrochloric acid (10 ml) and brine. The organic layer was dried over anhydrous sodium sulfate, and the solvent was evaporated under reduced pressure. The residue was subjected to silica gel column chromatography, and the fraction eluted with ethyl acetate-hexane (1:4) was concentrated under reduced pressure. The residue, bis(pinacolato)diboron (1.5 g), [1,1′-bis(diphenylphosphino)ferrocene]dichloropalladium(II) dichloromethane adduct (200 g) and potassium acetate (4 g) were dissolved in THF (40 ml), and the solution was stirred at refluxing temperature for 20 hr. To the reaction mixture was added ethyl acetate-water (2:1), and the insoluble material was filtered off. The filtrate was washed with saturated brine, and dried over anhydrous magnesium sulfate, and the solvent was evaporated under reduced pressure. The residue was subjected to silica gel column chromatography, and the fraction eluted with ethyl acetate-hexane (1:4) was concentrated under reduced pressure. The residue was dissolved in acetone (20 ml), and a solution of potassium peroxymonosulfate (3.0 g) in water (20 ml) was added at room temperature. The mixture was stirred at room temperature for 10 min, aqueous saturated thiosodium sulfate solution (20 ml) was added, and the liberated oil was extracted with ethyl acetate. The extract was washed successively with water and saturated brine, and dried over anhydrous sodium sulfate, and the solvent was evaporated under reduced pressure. The residue was subjected to reversed-phase preparative HPLC (the purification conditions are described above). The object fraction was neutralized with saturated aqueous sodium hydrogen carbonate, and the mixture was extracted with ethyl acetate. The extract was dried over anhydrous sodium sulfate, and the solvent was evaporated under reduced pressure to give 2-ethyl-1,3-benzothiazol-5-ol (324 mg) and 2-isopropyl-1,3-benzothiazol-5-ol (245 mg) as an amorphous solid, respectively.

### 2-Ethyl-1,3-benzothiazol-5-ol

¹H-NMR (CDCl₃) δ 1.48 (3H, t), 3.21 (2H, q), 6.77 (1H, br s), 6.99 (1H, dd), 7.47-7.72 (2H, m)

### 2-Isopropyl-1,3-benzothiazol-5-ol

¹H-NMR (CDCl₃) δ 1.50 (6H, d), 3.54 (1H, dt), 5.46 (1H, br s), 6.98 (1H, dd), 7.53 (1H, d), 7.63 (1H, d).

### Reference Example 919

### 1-tert-Butyl 4-benzyl (2R)-2-(2-{[1-(3-methoxypropyl)-2-oxo-1,2,3,4-tetrahydroquinolin-6-yl]oxy}ethyl)piperazine-1,4-dicarboxylate

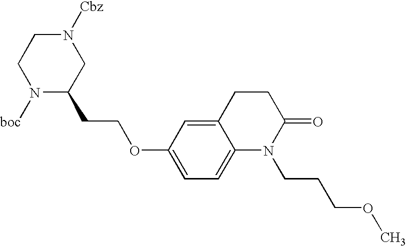

A solution of 1-tert-butyl 4-benzyl (2R)-2-{2-[(2-oxo-1,2,3,4-tetrahydroquinolin-6-yl)oxy]ethyl}piperazine-1,4-dicarboxylate (152 mg) in DMF (5 ml) was ice-cooled, sodium hydride (60% in oil) (12 mg) was added, and the mixture was stirred at room temperature for 30 min. 1-Bromo-3-methoxypropane (46 mg) was added, and the mixture was stirred for 2 hr, and poured into ice-cooled saturated aqueous sodium hydrogen carbonate. The mixture was extracted with ethyl acetate. The extract was dried over anhydrous sodium sulfate, and the solvent was evaporated under reduced pressure. The residue was subjected to silica gel column chromatography, and the fraction eluted with ethyl acetate-hexane (1:1) was concentrated under reduced pressure to give the object compound (220 mg) as an oil.

MS (ESI+, m/e) 582 (M+1)

In the same manner as in Reference Example 919, the following compound (Reference Example 920) was obtained.

### Reference Example 920

### 1-tert-Butyl 4-benzyl (2R)-2-(2-{[1-(2-methoxyethyl)-2-oxo-1,2,3,4-tetrahydroquinolin-6-yl]oxy}ethyl)piperazine-1,4-dicarboxylate

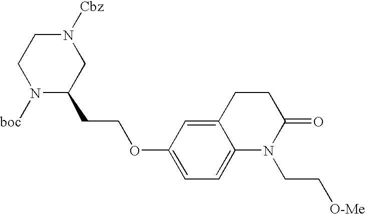

MS (ESI+, m/e) 568 (M+1)

In the same manner as in Reference Example 341, the following compounds (Reference Examples 921-948) were obtained.

### Reference Example 921

### 1-tert-Butyl 4-benzyl (2R)-2-[2-(3,4-dimethoxyphenoxy)ethyl]piperazine-1,4-dicarboxylate

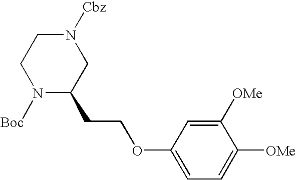

MS (ESI+, m/e) 401 (M+1-“Boc”)

### Reference Example 922

### 1-tert-Butyl 4-benzyl (2R)-2-[2-(3-methoxy-2-methylphenoxy)ethyl]piperazine-1,4-dicarboxylate

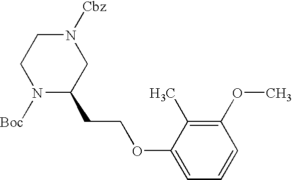

MS (ESI+, m/e) 385 (M+1-“Boc”)

### Reference Example 923

### 1-tert-Butyl 4-benzyl (2R)-2-[2-(2-fluoro-4-methylphenoxy)ethyl]piperazine-1,4-dicarboxylate

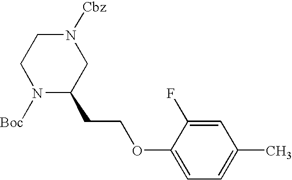

MS (ESI+, m/e) 473 (M+1)

### Reference Example 924

### 1-tert-Butyl 4-benzyl (2R)-2-[2-(2-chloro-4-methylphenoxy)ethyl]piperazine-1,4-dicarboxylate

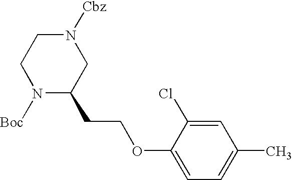

MS (ESI+, m/e) 489 (M+1)

### Reference Example 925

### 1-tert-Butyl 4-benzyl (2R)-2-[2-(4-chloro-2-fluorophenoxy)ethyl]piperazine-1,4-dicarboxylate

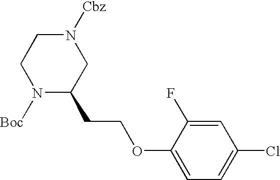

MS (ESI+, m/e) 393 (M+1-“Boc”)

### Reference Example 926

### 1-tert-Butyl 4-benzyl (2R)-2-[2-(2,3-dichlorophenoxy)ethyl]piperazine-1,4-dicarboxylate

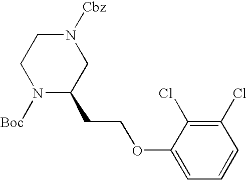

MS (ESI+, m/e) 409 (M+1-“Boc”)

### Reference Example 927

### 1-tert-Butyl 4-benzyl (2R)-2-[2-(2-fluoro-3-methoxyphenoxy)ethyl]piperazine-1,4-dicarboxylate

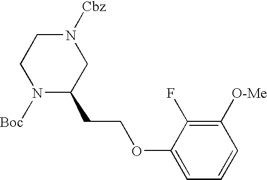

MS (ESI+, m/e) 489 (M+1)

### Reference Example 928

### 1-tert-Butyl 4-benzyl (2R)-2-{2-[3-(3-methoxypropoxy)phenoxy]ethyl}piperazine-1,4-dicarboxylate

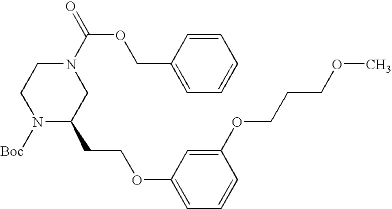

MS (ESI+, m/e) 529 (M+1)

### Reference Example 929

### 1-tert-Butyl 4-benzyl (2R)-2-{2-[3-(2-methoxyethoxy)phenoxy]ethyl}piperazine-1,4-dicarboxylate

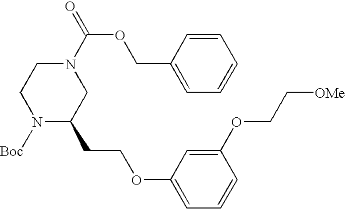

MS (ESI+, m/e) 515 (M+1)

### Reference Example 930

### 1-tert-Butyl 4-benzyl (2R)-2-[2-(2,3-dihydro-1-benzofuran-6-yloxy)ethyl]piperazine-1,4-dicarboxylate

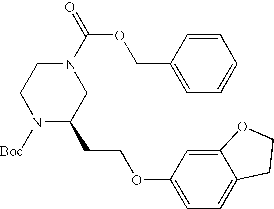

MS (ESI+, m/e) 483 (M+1)

### Reference Example 931

### 1-tert-Butyl 4-benzyl (2R)-2-[2-(5-methoxy-2-methylphenoxy)ethyl]piperazine-1,4-dicarboxylate

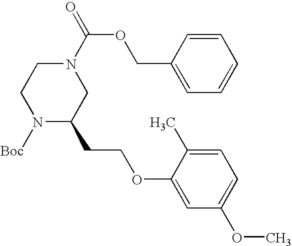

MS (ESI+, m/e) 485 (M+1)

### Reference Example 932

### 1-tert-Butyl 4-benzyl (2R)-2-[2-(5-chloro-2-methylphenoxy)ethyl]piperazine-1,4-dicarboxylate

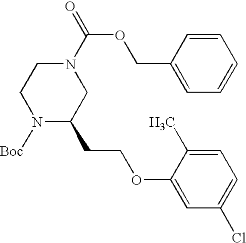

MS (ESI+, m/e) 490 (M+1)

### Reference Example 933

### 1-tert-Butyl 4-benzyl (2R)-2-[2-(2-chloro-5-methoxyphenoxy)ethyl]piperazine-1,4-dicarboxylate

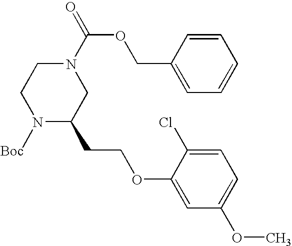

MS (ESI+, m/e) 506 (M+1)

### Reference Example 934

### 1-tert-Butyl 4-benzyl (2R)-2-[2-(2-chloro-4-methoxyphenoxy)ethyl]piperazine-1,4-dicarboxylate

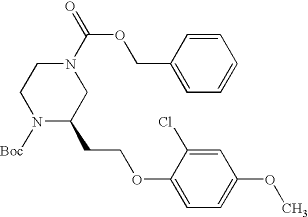

MS (ESI+, m/e) 506 (M+1)

### Reference Example 935

### 1-tert-Butyl 4-benzyl (2R)-2-{2-[(2-cyclopropyl-1,3-benzoxazol-5-yl)oxy]ethyl}piperazine-1,4-dicarboxylate

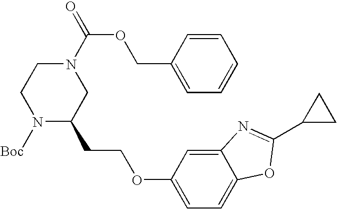

MS (ESI+, m/e) 522 (M+1)

### Reference Example 936

### 1-tert-Butyl 4-benzyl (2R)-2-{2-[(2,7-dimethyl-1,3-benzoxazol-6-yl)oxy]ethyl}piperazine-1,4-dicarboxylate

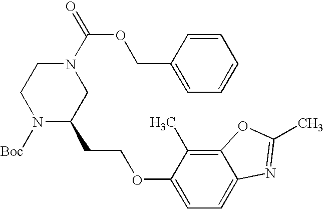

MS (ESI+, m/e) 510 (M+1)

### Reference Example 937

### 1-tert-Butyl 4-benzyl (2R)-2-{2-[(2-methyl-1,3-benzoxazol-6-yl)oxy]ethyl}piperazine-1,4-dicarboxylate

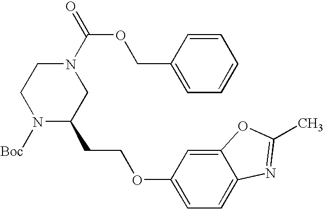

MS (ESI+, m/e) 496 (M+1)

### Reference Example 938

### 1-tert-Butyl 4-benzyl (2R)-2-{2-[(2-ethyl-1,3-benzoxazol-5-yl)oxy]ethyl}piperazine-1,4-dicarboxylate

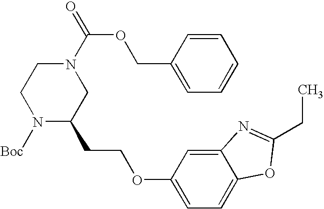

MS (ESI+, m/e) 510 (M+1)

### Reference Example 939

### 1-tert-Butyl 4-benzyl (2R)-2-{2-[(2-ethyl-7-methyl-1,3-benzoxazol-6-yl)oxy]ethyl}piperazine-1,4-dicarboxylate

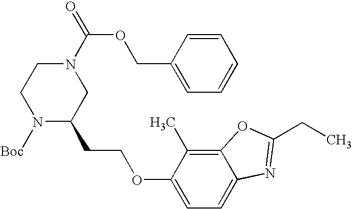

MS (ESI+, m/e) 524 (M+1)

### Reference Example 940

### 1-tert-Butyl 4-benzyl (2R)-2-{2-[(2-ethyl-1,3-benzoxazol-6-yl)oxy]ethyl}piperazine-1,4-dicarboxylate

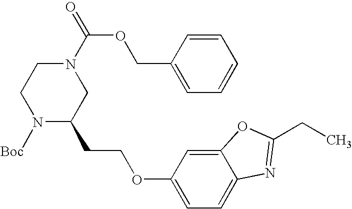

MS (ESI+, m/e) 510 (M+1)

### Reference Example 941

### 1-tert-Butyl 4-benzyl (2R)-2-{2-[(2-methyl-1,3-benzoxazol-5-yl)oxy]ethyl}piperazine-1,4-dicarboxylate

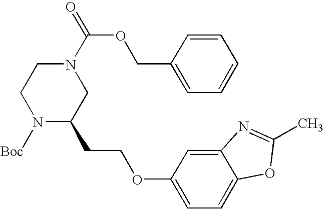

MS (ESI+, m/e) 496 (M+1)

### Reference Example 942

### 1-tert-Butyl 4-benzyl (2R)-2-{2-[3,5-bis(trifluoromethyl)phenoxy]ethyl}piperazine-1,4-dicarboxylate

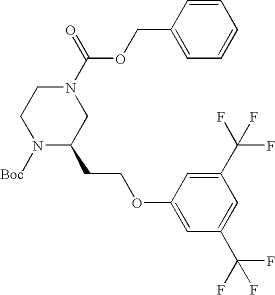

MS (ESI+, m/e) 577 (M+1)

### Reference Example 943

### 1-tert-Butyl 4-benzyl (2R)-2-{2-[3-fluoro-5-(trifluoromethyl)phenoxy]ethyl}piperazine-1,4-dicarboxylate

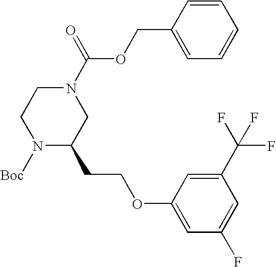

MS (ESI+, m/e) 527 (M+1)

### Reference Example 944

### 1-tert-Butyl 4-benzyl (2R)-2-[2-(3,5-difluorophenoxy)ethyl]piperazine-1,4-dicarboxylate

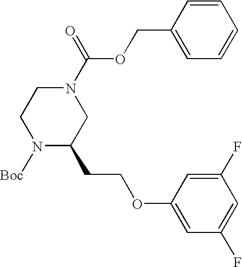

MS (ESI+, m/e) 427 (M+1)

### Reference Example 945

### 1-tert-Butyl 4-benzyl (2R)-2-(2-{[3-(2-methoxy-2-oxoethyl)-2,3-dihydro-1-benzofuran-5-yl]oxy}ethyl)piperazine-1,4-dicarboxylate

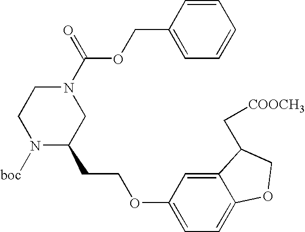

MS (ESI+, m/e) 555 (M+1)

### Reference Example 946

### 1-tert-Butyl 4-benzyl (2R)-2-[2-(4-tert-butylphenoxy)ethyl]piperazine-1,4-dicarboxylate

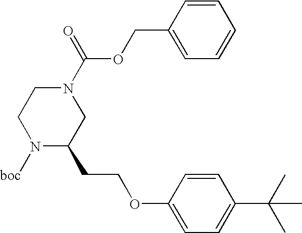

MS (ESI+, m/e) 497 (M+1)

### Reference Example 947

### 1-tert-Butyl 4-benzyl (2R)-2-[2-(3,4-dimethylphenoxy)ethyl]piperazine-1,4-dicarboxylate

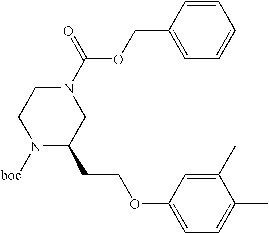

MS (ESI+, m/e) 469 (M+1)

### Reference Example 948

### 1-tert-Butyl 4-benzyl (2R)-2-{2-[4-isopropylphenoxy]ethyl}piperazine-1,4-dicarboxylate

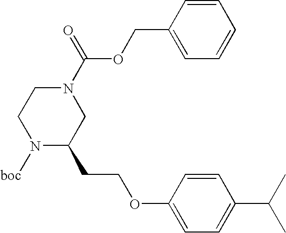

MS (ESI+, m/e) 483 (M+1)

In the same manner as in Reference Example 663, the following compounds (Reference Examples 949-951) were obtained.

### Reference Example 949

### 1-tert-Butyl 4-benzyl (2R)-2-{2-[(5-chloro-2-methylphenyl)amino]ethyl}piperazine-1,4-dicarboxylate

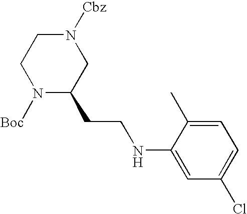

MS (ESI+, m/e) 488 (M+1)

### Reference Example 950

### 1-tert-Butyl 4-benzyl (2R)-2-{2-[(2-fluoro-3-methoxyphenyl)amino]ethyl}piperazine-1,4-dicarboxylate

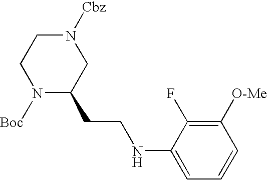

MS (ESI+, m/e) 488 (M+1)

### Reference Example 951

### 1-tert-Butyl 4-benzyl (2R)-2-[2-(2,3-dihydrofuro[3,2-b]pyridin-5-ylamino)ethyl]piperazine-1,4-dicarboxylate

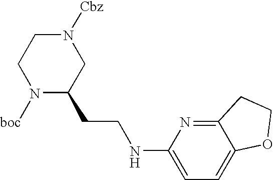

MS (ESI+, m/e) 483 (M+1)

In the same manner as in Reference Example 383, the following compounds (Reference Examples 952-981) were obtained.

### Reference Example 952

### Benzyl (3R)-3-(2-{[1-(3-methoxypropyl)-2-oxo-1,2,3,4-tetrahydroquinolin-6-yl]oxy}ethyl)piperazine-1-carboxylate

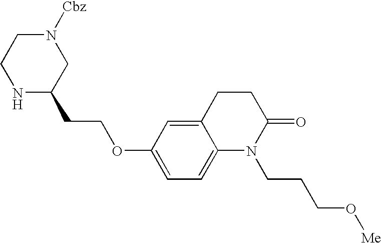

MS (ESI+, m/e) 482 (M+1)

### Reference Example 953

### Benzyl (3R)-3-(2-{[1-(2-methoxyethyl)-2-oxo-1,2,3,4-tetrahydroquinolin-6-yl]oxy}ethyl)piperazine-1-carboxylate

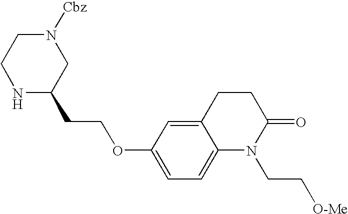

MS (ESI+, m/e) 468 (M+1)

### Reference Example 954

### Benzyl (3R)-3-[2-(3,4-dimethoxyphenoxy)ethyl]piperazine-1-carboxylate

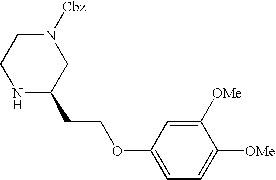

MS (ESI+, m/e) 401 (M+1)

### Reference Example 955

### Benzyl (3R)-3-[2-(3-methoxy-2-methylphenoxy)ethyl]piperazine-1-carboxylate

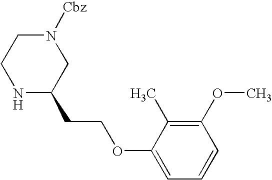

MS (ESI+, m/e) 385 (M+1)

### Reference Example 956

### Benzyl (3R)-3-[2-(2-fluoro-4-methylphenoxy)ethyl]piperazine-1-carboxylate

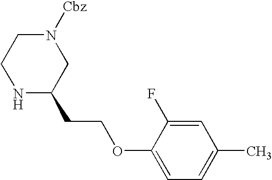

MS (ESI+, m/e) 373 (M+1)

### Reference Example 957

### Benzyl (3R)-3-[2-(2-chloro-4-methylphenoxy)ethyl]piperazine-1-carboxylate

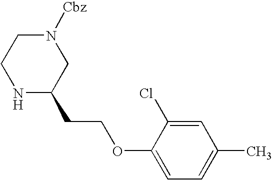

MS (ESI+, m/e) 389 (M+1)

### Reference Example 958

### Benzyl (3R)-3-[2-(4-chloro-2-fluorophenoxy)ethyl]piperazine-1-carboxylate

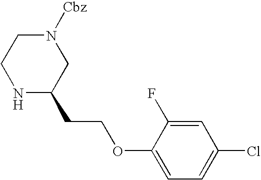

MS (ESI+, m/e) 393 (M+1)

### Reference Example 959

### Benzyl (3R)-3-[2-(2,3-dichlorophenoxy)ethyl]piperazine-1-carboxylate

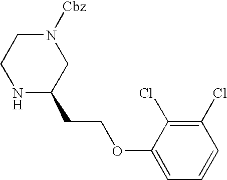

MS (ESI+, m/e) 409 (M+1)

### Reference Example 960

### Benzyl (3R)-3-[2-(2-fluoro-3-methoxyphenoxy)ethyl]piperazine-1-carboxylate

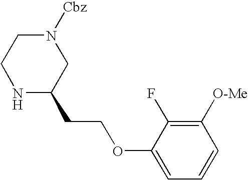

MS (ESI+, m/e) 389 (M+1)

### Reference Example 961

### Benzyl (3R)-3-{2-[3-(3-methoxypropoxy)phenoxy]ethyl}piperazine-1-carboxylate

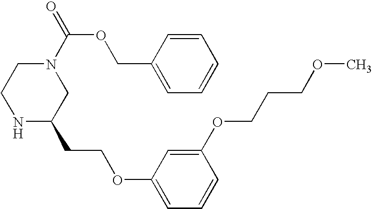

MS (ESI+, m/e) 429 (M+1)

### Reference Example 962

### Benzyl (3R)-3-{2-[3-(2-methoxyethoxy)phenoxy]ethyl}piperazine-1-carboxylate

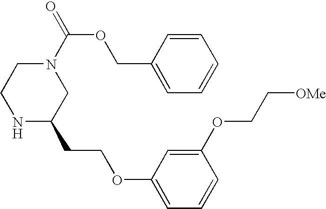

MS (ESI+, m/e) 415 (M+1)

### Reference Example 963

### Benzyl (3R)-3-[2-(2,3-dihydro-1-benzofuran-6-yloxy)ethyl]piperazine-1-carboxylate

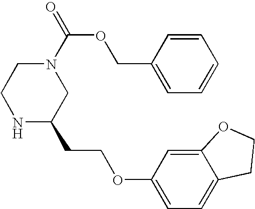

MS (ESI+, m/e) 383 (M+1)

### Reference Example 964

### Benzyl (3R)-3-[2-(5-methoxy-2-methylphenoxy)ethyl]piperazine-1-carboxylate

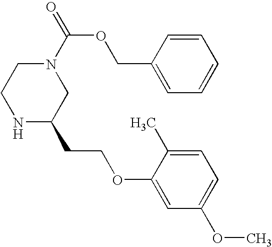

MS (ESI+, m/e) 385 (M+1)

### Reference Example 965

### Benzyl (3R)-3-[2-(5-chloro-2-methylphenoxy)ethyl]piperazine-1-carboxylate

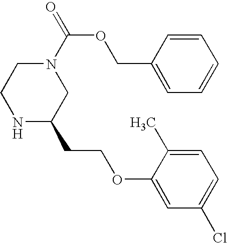

MS (ESI+, m/e) 390 (M+1)

### Reference Example 966

### Benzyl (3R)-3-[2-(2-chloro-5-methoxyphenoxy)ethyl]piperazine-1-carboxylate

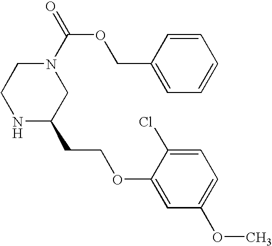

MS (ESI+, m/e) 406 (M+1)

### Reference Example 967

### Benzyl (3R)-3-[2-(2-chloro-4-methoxyphenoxy)ethyl]piperazine-1-carboxylate

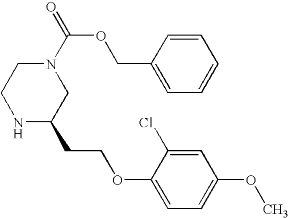

MS (ESI+, m/e) 406 (M+1)

### Reference Example 968

### Benzyl (3R)-3-{2-[(2-cyclopropyl-1,3-benzoxazol-5-yl)oxy]ethyl}piperazine-1-carboxylate

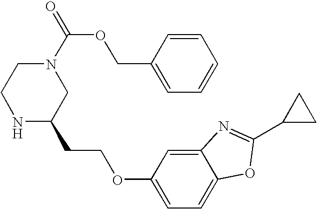

MS (ESI+, m/e) 422 (M+1)

### Reference Example 969

### Benzyl (3R)-3-{2-[(2,7-dimethyl-1,3-benzoxazol-6-yl)oxy]ethyl}piperazine-1-carboxylate

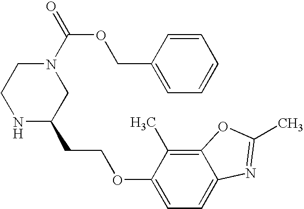

MS (ESI+, m/e) 410 (M+1)

### Reference Example 970

### Benzyl (3R)-3-{2-[(2-methyl-1,3-benzoxazol-6-yl)oxy]ethyl}piperazine-1-carboxylate

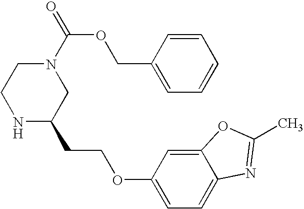

MS (ESI+, m/e) 396 (M+1)

### Reference Example 971

### Benzyl (3R)-3-{2-[(2-ethyl-1,3-benzoxazol-5-yl)oxy]ethyl}piperazine-1-carboxylate

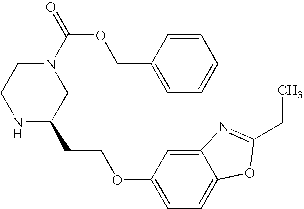

MS (ESI+, m/e) 410 (M+1)

### Reference Example 972

### Benzyl (3R)-3-{2-[(2-ethyl-7-methyl-1,3-benzoxazol-6-yl)oxy]ethyl}piperazine-1-carboxylate

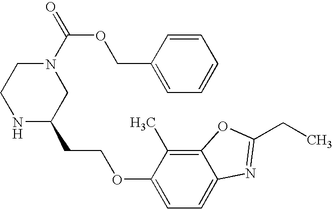

MS (ESI+, m/e) 424 (M+1)

### Reference Example 973

### Benzyl (3R)-3-{2-[(2-ethyl-1,3-benzoxazol-6-yl)oxy]ethyl}piperazine-1-carboxylate

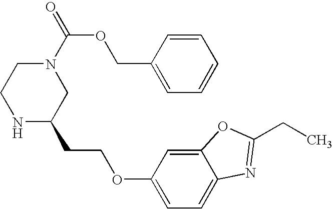

MS (ESI+, m/e) 410 (M+1)

### Reference Example 974

### Benzyl (3R)-3-{2-[(2-methyl-1,3-benzoxazol-5-yl)oxy]ethyl}piperazine-1-carboxylate

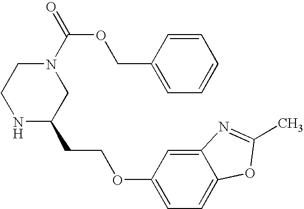

MS (ESI+, m/e) 396 (M+1)

### Reference Example 975

### Benzyl (3R)-3-{2-[3,5-bis(trifluoromethyl)phenoxy]ethyl}piperazine-1-carboxylate

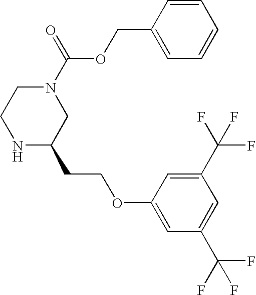

MS (ESI+, m/e) 477 (M+1)

### Reference Example 976

### Benzyl (3R)-3-{2-[3-fluoro-5-(trifluoromethyl)phenoxy]ethyl}piperazine-1-carboxylate

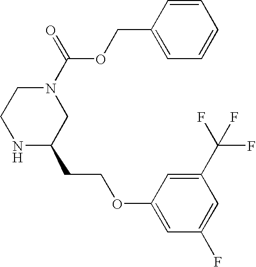

MS (ESI+, m/e) 427 (M+1)

### Reference Example 977

### Benzyl (3R)-3-[2-(3,5-difluorophenoxy)ethyl]piperazine-1-carboxylate

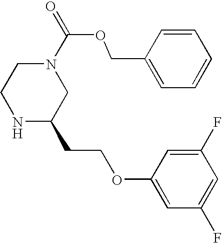

MS (ESI+, m/e) 327 (M+1)

### Reference Example 978

### Benzyl (3R)-3-(2-{[3-(2-methoxy-2-oxoethyl)-2,3-dihydro-1-benzofuran-5-yl]oxy}ethyl)piperazine-1-carboxylate

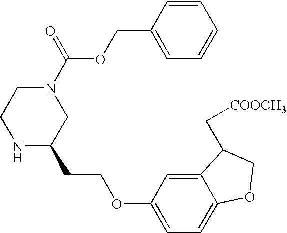

MS (ESI+, m/e) 455 (M+1)

### Reference Example 979

### Benzyl (3R)-3-[2-(4-tert-butylphenoxy)ethyl]piperazine-1-carboxylate

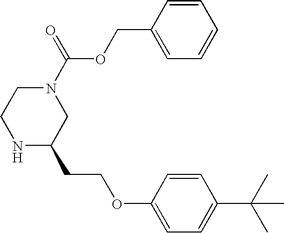

MS (ESI+, m/e) 397 (M+1)

### Reference Example 980

### Benzyl (3R)-3-[2-(3,4-dimethylphenoxy)ethyl]piperazine-1-carboxylate

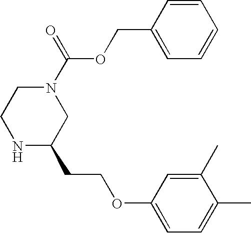

MS (ESI+, m/e) 369 (M+1)

### Reference Example 981

### Benzyl (3R)-3-{2-[4-isopropylphenoxy]ethyl}piperazine-1-carboxylate

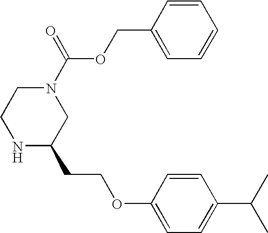

MS (ESI+, m/e) 383 (M+1)

### Reference Example 982

### Benzyl (3R)-3-{2-[(5-chloro-2-methylphenyl)amino]ethyl}piperazine-1-carboxylate

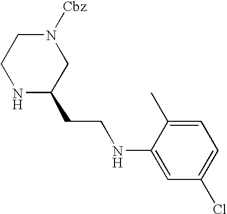

MS (ESI+, m/e) 388 (M+1)

### Reference Example 983

### Benzyl (3R)-3-{2-[(2-fluoro-3-methoxyphenyl)amino]ethyl}piperazine-1-carboxylate

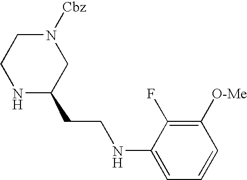

MS (ESI+, m/e) 388 (M+1)

### Reference Example 984

### Benzyl (3R)-3-[2-(2,3-dihydrofuro[3,2-b]pyridin-5-ylamino)ethyl]piperazine-1-carboxylate

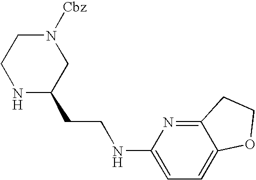

MS (ESI+, m/e) 383 (M+1)

In the same manner as in Reference Example 243, the following compound (Reference Example 985) was obtained.

### Reference Example 985

### tert-Butyl (3S)-3-[(5-phenyl-2H-tetrazol-2-yl)methyl]piperazine-1-carboxylate

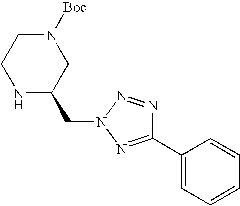

MS (ESI+, m/e) 345 (M+1)

In the same manner as in Reference Example 806, the following compounds (Reference Examples 986-988) were obtained.

### Reference Example 986

### 2-[(2R)-4-Benzyl-3,6-dioxopiperazin-2-yl]-N-(2,5-dimethylphenyl)acetamide

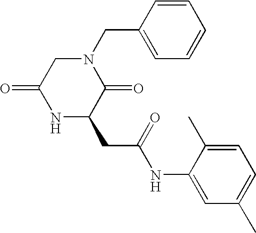

MS (ESI+, m/e) 366 (M+1)

### Reference Example 987

### 2-[(2R)-4-Benzyl-3,6-dioxopiperazin-2-yl]-N-(5-fluoro-2-methylphenyl)acetamide

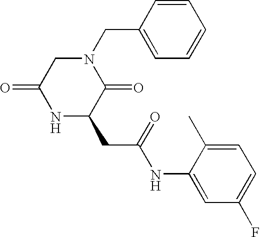

MS (ESI+, m/e) 370 (M+1)

### Reference Example 988

### 2-[(2R)-4-Benzyl-3,6-dioxopiperazin-2-yl]-N-(2-fluoro-3-methoxyphenyl)acetamide

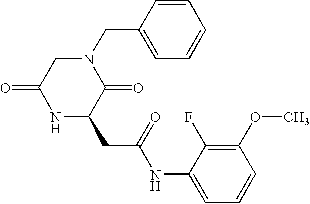

MS (ESI+, m/e) 386 (M+1)

In the same manner as in Reference Example 827, the following compounds (Reference Examples 989-991) were obtained.

### Reference Example 989

### N-{2-[(2R)-4-Benzylpiperazin-2-yl]ethyl}-2,5-dimethylaniline

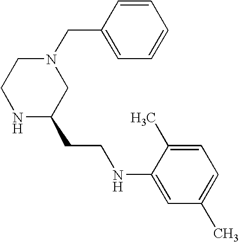

MS (ESI+, m/e) 324 (M+1)

### Reference Example 990

### N-{2-[(2R)-4-Benzylpiperazin-2-yl]ethyl}-5-fluoro-2-methylaniline

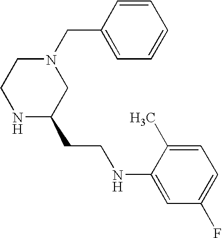

MS (ESI+, m/e) 328 (M+1)

### Reference Example 991

### N-{2-[(2R)-4-Benzylpiperazin-2-yl]ethyl}-2-fluoro-3-methoxyaniline

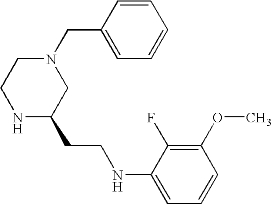

MS (ESI+, m/e) 344 (M+1)

In the same manner as in Reference Example 255, the following compounds (Reference Examples 992-995) were obtained.

### Reference Example 992

### 1-tert-Butyl 4-benzyl (2R)-2-[2-(2,6-difluorophenoxy)ethyl]piperazine-1,4-dicarboxylate

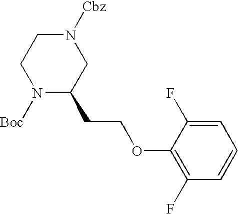

MS (ESI+, m/e) 377 (M+1-Boc)

### Reference Example 993

### 1-tert-Butyl 4-benzyl (2R)-2-[2-(naphthalen-2-yloxy)ethyl]piperazine-1,4-dicarboxylate

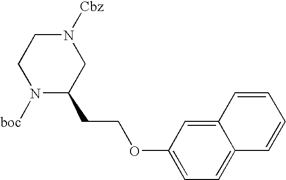

MS (ESI+, m/e) 391 (M+1-Boc)

### Reference Example 994

### 1-tert-Butyl 4-benzyl (2R)-2-[2-(4-methylphenoxy)ethyl]piperazine-1,4-dicarboxylate

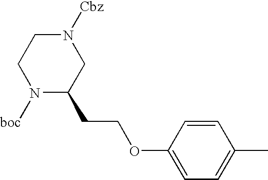

MS (ESI+, m/e) 355 (M+1-Boc)

### Reference Example 995

### 1-tert-Butyl 4-benzyl (2R)-2-[2-(3-methoxy-4-methylphenoxy)ethyl]piperazine-1,4-dicarboxylate

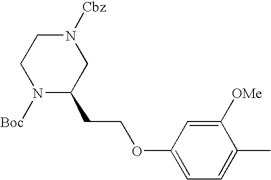

MS (ESI+, m/e) 385 (M+1-Boc)

In the same manner as in Reference Example 383, the following compounds (Reference Examples 996-999) were obtained.

### Reference Example 996

### Benzyl (3R)-3-[2-(2,6-difluorophenoxy)ethyl]piperazine-1-carboxylate

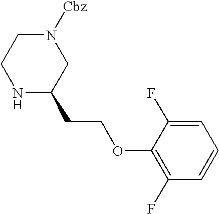

MS (ESI+, m/e) 377 (M+1)

### Reference Example 997

### Benzyl (3R)-3-[2-(naphthalen-2-yloxy)ethyl]piperazine-1-carboxylate

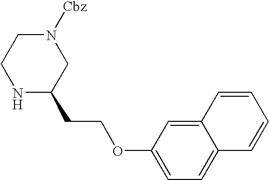

MS (ESI+, m/e) 391 (M+1)

### Reference Example 998

### Benzyl (3R)-3-[2-(4-methylphenoxy)ethyl]piperazine-1-carboxylate

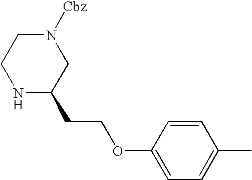

MS (ESI+, m/e) 385 (M+1)

### Reference Example 999

### Benzyl (3R)-3-[2-(3-methoxy-4-methylphenoxy)ethyl]piperazine-1-carboxylate

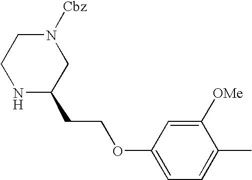

MS (ESI+, m/e) 385 (M+1)

### Reference Example 1000

### Benzyl (3R)-3-[2-(2,3-dihydro-1H-inden-2-yloxy)ethyl]piperazine-1-carboxylate

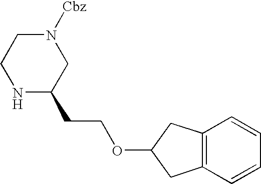

2,3-Dihydro-1H-inden-2-ol (161 mg) was dissolved in DMF (5 ml), sodium hydride (60% in oil) (60 mg) was added, and the mixture was stirred at room temperature for 1 hr. 1-tert-Butyl 4-benzyl (2R)-2-{2-[(methylsulfonyl)oxy]ethyl}piperazine-1,4-dicarboxylate (443 mg) was added thereto, and the mixture was stirred at 60° C. for 15 hr. The reaction mixture was poured into aqueous sodium bicarbonate solution, and the mixture was extracted with ethyl acetate. The extract was washed with saturated brine, dried over anhydrous magnesium sulfate, and concentrated. The residue was subjected to silica gel column chromatography, and the fraction eluted with ethyl acetate-hexane (6:4) was concentrated under reduced pressure to give 1-tert-butyl 4-benzyl (2R)-2-[2-(2,3-dihydro-1H-inden-2-yloxy)ethyl]piperazine-1,4-dicarboxylate (252 mg) as an oil. The obtained 1-tert-butyl 4-benzyl (2R)-2-[2-(2,3-dihydro-1H-inden-2-yloxy)ethyl]piperazine-1,4-dicarboxylate (252 mg) was dissolved in methanol (5 ml), 4N hydrogen chloride-ethyl acetate solution was added. The mixture was stirred at room temperature for 5 hr, and concentrated to give the object compound (157 mg).

MS (ESI+, m/e) 381 (M+1)

In the same manner as in Reference Example 529, the following compound (Reference Example 1001) was obtained.

### Reference Example 1001

### tert-Butyl [(1S,2S)-2-(4-{[(2R)-4-benzyl-2-(3,5-difluorobenzyl)piperazin-1-yl]carbonyl}-5-phenyl-1H-imidazol-1-yl)cyclohexyl]carbamate

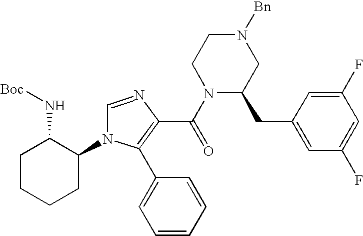

MS (ESI+, m/e) 670 (M+1)

### Reference Example 1002

### (1S,2S)-2-(4-{[(2R)-4-Benzyl-2-(3,5-difluorobenzyl)piperazin-1-yl]carbonyl}-5-phenyl-1H-imidazol-1-yl)cyclohexanamine

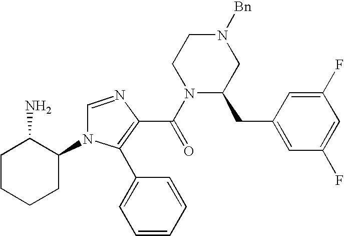

tert-Butyl [(1S,2S)-2-(4-{[(2R)-4-benzyl-2-(3,5-difluorobenzyl)piperazin-1-yl]carbonyl}-5-phenyl-1H-imidazol-1-yl)cyclohexyl]carbamate (5.04 g) was dissolved in methanol (10 ml), 4N hydrogen chloride-ethyl acetate solution was added, and the mixture was stirred at room temperature for 5 hr, and concentrated. Aqueous sodium bicarbonate solution was added, and the mixture was extracted with ethyl acetate. The extract was washed with saturated brine, dried over anhydrous magnesium sulfate, and concentrated to give the object compound (4.02 g).

MS (ESI+, m/e) 570 (M+1)

### Reference Example 1003

### 1-[(1S,2S)-2-{[(Cyclobutyloxy)carbonyl]amino}cyclohexyl]-5-phenyl-1H-imidazole-4-carboxylic acid

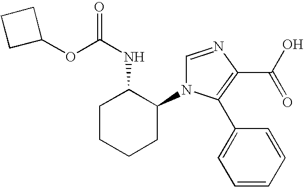

Ethyl 1-[(1S,2S)-2-aminocyclohexyl]-5-phenyl-1H-imidazole-4-carboxylate (1.57 g) and DMAP (916 mg) were dissolved in THF (50 ml), and the solution was ice-cooled. 4-Nitrophenyl chloroformate (1.21 g) was added, and the mixture was stirred at 0° C. for 1 hr, and then at room temperature for 2 hr. To the reaction mixture was added cyclobutanol (0.77 ml), and the mixture was stirred at 60° C. for 15 hr. The reaction mixture was poured into 1N aqueous sodium hydroxide solution, and the mixture was extracted with ethyl acetate. The extract was washed with saturated brine, dried over anhydrous magnesium sulfate, and concentrated under reduced pressure. The residue was subjected to basic silica gel column chromatography, and the fraction eluted with ethyl acetate was concentrated under reduced pressure to give ethyl 1-[(1S,2S)-2-{[(cyclobutyloxy)carbonyl]amino}cyclohexyl]-5-phenyl-1H-imidazole-4-carboxylate (1.22 g) as an amorphous solid. The obtained ethyl 1-[(1S,2S)-2-{[(cyclobutyloxy)carbonyl]amino}cyclohexyl]-5-phenyl-1H-imidazole-4-carboxylate (1.22 g) was dissolved in ethanol (30 ml), 2N aqueous sodium hydroxide solution (14.8 ml) was added, and the mixture was stirred at 60° C. for 15 hr. After cooling to room temperature, the mixture was neutralized (pH 7) with diluted hydrochloric acid, and the solvent was evaporated under reduced pressure. The residue was suspended in ethanol (100 ml), and the insoluble material was filtered off. The filtrate was concentrated under reduced pressure to give the object compound (1.20 g) as a powder mixed with an inorganic salt thereof.

NMR (DMSO-d₆) δ: 0.84-1.13 (1H, m), 1.36 (3H, br. s.), 1.42-2.01 (8H, m), 2.04-2.26 (2H, m), 3.11-3.24 (1H, m), 3.70-3.97 (1H, m), 4.67 (1H, t, J=7.5), 7.12 (1H, d, J=9.0), 7.29-7.40 (2H, m), 7.39-7.59 (3H, m), 8.31 (1H, s), 12.23 (1H, br. s.).

### Reference Example 1004

### 1-[(1S,2S)-2-{[(2-Methoxyethoxy)carbonyl]amino}cyclohexyl]-5-phenyl-1H-imidazole-4-carboxylic acid

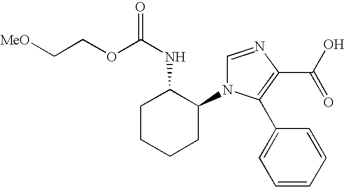

Ethyl 1-[(1S,2S)-2-aminocyclohexyl]-5-phenyl-1H-imidazole-4-carboxylate (940 mg) and triethylamine (0.836 ml) were dissolved in THF (50 ml), 2-methoxyethyl chlorocarbonate (499 mg) was added, and the mixture was stirred at room temperature for 15 hr. To the reaction mixture was added aqueous sodium bicarbonate, and the mixture was extracted with ethyl acetate. The extract was washed with saturated brine, dried over anhydrous magnesium sulfate, and concentrated. The residue was subjected to basic silica gel column chromatography, and the fraction eluted with ethyl acetate was concentrated under reduced pressure to give ethyl 1-[(1S,2S)-2-{[(2-methoxyethoxy)carbonyl]amino}cyclohexyl]-5-phenyl-1H-imidazole-4-carboxylate (1.20 g). The obtained ethyl 1-[(1S,2S)-2-{[(2-methoxyethoxy)carbonyl]amino}cyclohexyl]-5-phenyl-1H-imidazole-4-carboxylate (1.20 g) was dissolved in methoxyethanol (30 ml), 2N aqueous sodium hydroxide solution (14.5 ml) was added, and the mixture was stirred at 60° C. for 15 hr. After cooling to room temperature, the mixture was neutralized (pH 7) with diluted hydrochloric acid, and the solvent was evaporated under reduced pressure. The residue was suspended in ethanol (100 ml), and the insoluble material was filtered off. The filtrate was concentrated under reduced pressure to give the object compound (1.23 g) as a powder mixed with an inorganic salt thereof.

NMR (CDCl₃) δ: 0.93-1.48 (4H, m), 1.48-2.08 (4H, m), 2.08-2.56 (2H, m), 2.94-4.10 (8H, m), 6.76-7.89 (6H, m).

In the same manner as in Reference Example 1004, the following compound (Reference Example 1005) was obtained.

### Reference Example 1005

### 1-{(1S,2S)-2-[(Methylsulfonyl)amino]cyclohexyl}-5-phenyl-1H-imidazole-4-carboxylic acid

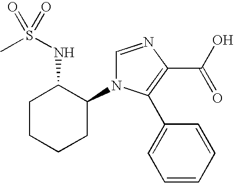

NMR (DMSO-d₆) δ: 0.86-1.07 (1H, m), 1.14-1.46 (1H, m), 1.62 (3H, d, J=9.8), 1.70-1.91 (2H, m), 2.56 (3H, s), 2.96-3.63 (2H, m), 3.63-3.86 (1H, m), 7.10 (1H, d, J=9.1), 7.27-7.39 (2H, m), 7.38-7.47 (3H, m), 7.98 (1H, s).

In the same manner as in Reference Example 39, the following compound (Reference Example 1006) was obtained.

### Reference Example 1006

### Ethyl 1-{(1S,2S)-2-[(isopropoxycarbonyl)amino]cyclohexyl}-5-phenyl-1H-imidazole-4-carboxylate

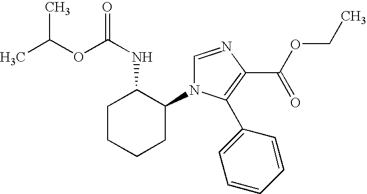

¹H-NMR (CDCl₃) 6: ppm 1.12-1.23 (11H, m), 1.34-1.46 (1H, m), 1.73-1.86 (3H, m), 2.02-2.10 (2H, m), 3.46-3.53 (1H, m), 3.85 (1H, brs), 4.09-4.13 (1H, m), 4.20 (2H, q), 4.72-4.80 (1H, m), 7.30-7.32 (2H, m), 7.48-7.51 (3H, m), 7.73 (1H, s).

In the same manner as in Reference Example 66, the following compound (Reference Example 1007) was obtained.

### Reference Example 1007

### 1-{(1S,2S)-2-[(Isopropoxycarbonyl)amino]cyclohexyl}-5-phenyl-1H-imidazole-4-carboxylic acid

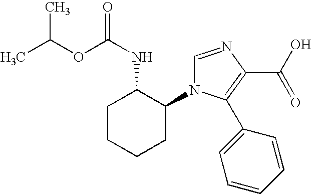

¹H-NMR (DMSO-d₆) 6: ppm 1.08 (6H, dd), 1.07-1.09 (1H, m), 1.24-1.35 (2H, m), 1.63-1.78 (3H, m), 1.94-2.07 (2H, m), 3.49-3.58 (1H, m), 3.86-3.88 (1H, m), 4.53-4.61 (1H, m), 7.15 (1H, d), 7.39-7.41 (2H, m), 7.54-7.57 (3H, m), 9.40 (1H, brs), 11.99 (1H, brs).

### Example 1

### Method A

### (1R,2S)-2-(4-{[(2R)-2-(3,5-Difluorobenzyl)piperazin-1-yl]carbonyl}-5-phenyl-1H-imidazol-1-yl)cyclohexanol hydrochloride

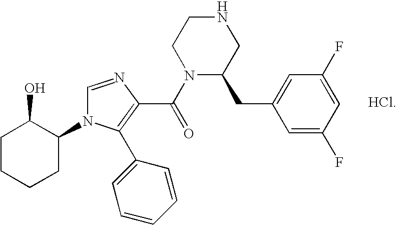

A solution of 1-[(1S,2R)-2-hydroxycyclohexyl]-5-phenyl-1H-imidazole-4-carboxylic acid (129 mg), (3R)-1-benzyl-3-(3,5-difluorobenzyl)piperazine (142 mg), WSC.HCl (104 mg) and HOBt (73 mg) in DMF (3 ml) was stirred at room temperature for 15 hr, and poured into saturated aqueous sodium hydrogen carbonate, and the mixture was extracted with ethyl acetate. The extract was washed successively with water and saturated brine, and dried over anhydrous magnesium sulfate, and the solvent was evaporated under reduced pressure. The residue was subjected to basic silica gel column chromatography, and the fraction eluted with ethyl acetate-hexane (1:1-1:0) was concentrated under reduced pressure to give (1R,2S)-2-(4-{[(2R)-4-benzyl-2-(3,5-difluorobenzyl)piperazin-1-yl]carbonyl}-5-phenyl-1H-imidazol-1-yl)cyclohexanol (205 mg) as an amorphous solid. The total amount thereof was dissolved in methanol (6 ml), 20% palladium hydroxide-carbon (50% containing water, 105 mg) was added thereto, and the mixture was subjected to catalytic reduction at ambient temperature and normal pressure for 15 hr. The catalyst was filtered off, and the filtrate was concentrated under reduced pressure. The residue was subjected to basic silica gel column chromatography, and the fraction eluted with ethyl acetate-methanol (50:1-10:1) was concentrated under reduced pressure. The residue was diluted with diethyl ether (2 ml), 4N hydrogen chloride-ethyl acetate solution (99 μl) was added, and the precipitated crystals were collected by filtration to give the object compound (89 mg).

MS (ESI+, m/e) 481 (M+1)

### Example 2

### Method B

### (2R)-2-Benzyl-1-({1-[(1R,2R)-2-(benzyloxy)cyclopentyl]-5-phenyl-1H-imidazol-4-yl}carbonyl)piperazine

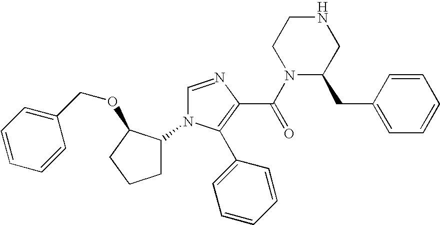

A solution of 1-[(1R,2R)-2-(benzyloxy)cyclopentyl]-5-phenyl-1H-imidazole-4-carboxylic acid (460 mg), tert-butyl (3R)-3-benzylpiperazine-1-carboxylate (368 mg), WSC.HCl (292 mg) and HOBt (206 mg) in DMF (8 ml) was stirred at room temperature for 15 hr, and poured into saturated aqueous sodium hydrogen carbonate, and the mixture was extracted with ethyl acetate. The extract was washed successively with water and saturated brine, and dried over anhydrous magnesium sulfate, and the solvent was evaporated under reduced pressure. The residue was subjected to basic silica gel column chromatography, and the fraction eluted with ethyl acetate-hexane (1:9-1:0) was concentrated under reduced pressure to give tert-butyl (3R)-3-benzyl-4-({1-[(1R,2R)-2-(benzyloxy)cyclopentyl]-5-phenyl-1H-imidazol-4-yl}carbonyl)piperazine-1-carboxylate (401 mg) as an amorphous solid. 200 mg therefrom was dissolved in dichloromethane (1 ml), TFA (1 ml) was added thereto, and the mixture was stirred at room temperature for 30 min. After stirring, the mixture was concentrated under reduced pressure. The residue was dissolved in ethyl acetate, and the solution was washed successively with saturated aqueous sodium hydrogen carbonate and saturated brine, and dried over anhydrous sodium sulfate. The solvent was evaporated under reduced pressure to give the object compound (152 mg).

MS (ESI+, m/e) 521 (M+1)

### Example 3

### Method C

### (1S,2R)-2-(4-{[(2R)-2-(2,4-Dichlorobenzyl)piperazin-1-yl]carbonyl}-5-phenyl-1H-imidazol-1-yl)-1-(methoxymethyl)cyclohexanol hydrochloride

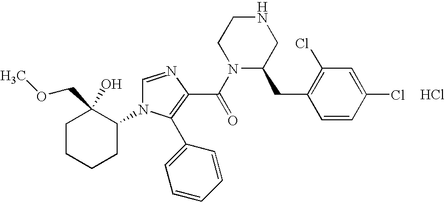

A solution of 1-[(1R,2S)-2-hydroxy-2-(methoxymethyl)cyclohexyl]-5-phenyl-1H-imidazole-4-carboxylic acid (132 mg), tert-butyl (3R)-3-(2,4-dichlorobenzyl)piperazine-1-carboxylate (145 mg), WSC.HCl (92 mg) and HOBt (65 mg) in DMF (3.5 ml) was stirred at room temperature for 15 hr, and poured into saturated aqueous sodium hydrogen carbonate, and the mixture was extracted with ethyl acetate. The extract was washed successively with water and saturated brine, and dried over anhydrous magnesium sulfate, and the solvent was evaporated under reduced pressure. The residue was subjected to silica gel column chromatography, and the fraction eluted with ethyl acetate-hexane (1:1-1:0) was concentrated under reduced pressure to give tert-butyl (3R)-3-(2,4-dichlorobenzyl)-4-({1-[(1R,2S)-2-hydroxy-2-(methoxymethyl)cyclohexyl]-5-phenyl-1H-imidazol-4-yl}carbonyl)piperazine-1-carboxylate (194 mg) as an amorphous solid. The total amount thereof was dissolved in ethyl acetate (1 ml), 4N hydrogen chloride-ethyl acetate solution (1 ml) was added thereto, and the mixture was stirred at room temperature for 2 hr. The reaction mixture was diluted with diethyl ether (8 ml), and the precipitated crystals were collected by filtration to give the object compound (93 mg).

MS (ESI+, m/e) 557 (M+1)

### Example 4

### Method D

### 1-[(4-{[(2R)-2-Benzylpiperazin-1-yl]carbonyl}-5-phenyl-1H-imidazol-1-yl)methyl]cyclohexanol

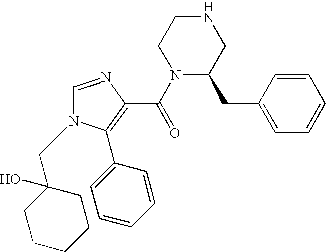

A mixture of ethyl 1-[(1-hydroxycyclohexyl)methyl]-5-phenyl-1H-imidazole-4-carboxylate (440 mg), lithium hydroxide monohydrate (100 mg), ethanol (3 ml) and water (3 ml) was stirred at 60° C. for 10 hr, and concentrated under reduced pressure. The residue was mixed with tert-butyl (3R)-3-benzylpiperazine-1-carboxylate (440 mg), WSC.HCl (640 mg), HOBt (1.00 g) and DMF (7 ml). The mixture was stirred at 50° C. for 3 hr, and poured into saturated aqueous sodium hydrogen carbonate, and the mixture was extracted with ethyl acetate. The extract was washed with saturated brine, and dried over anhydrous magnesium sulfate, and the solvent was evaporated under reduced pressure. The residue was subjected to basic silica gel column chromatography, and the fraction eluted with ethyl acetate-hexane (1:4-1:0) was concentrated under reduced pressure to give tert-butyl (3R)-3-benzyl-4-({1-[(1-hydroxycyclohexyl)methyl]-5-phenyl-1H-imidazol-4-yl}carbonyl)piperazine-1-carboxylate (510 mg) as an amorphous solid. 200 mg therefrom was dissolved in dichloromethane (2 ml), and TFA (2 ml) was added thereto. The mixture was stirred at room temperature for 1 hr, and concentrated under reduced pressure. The residue was dissolved in ethyl acetate, and the solution was washed successively with saturated aqueous sodium hydrogen carbonate and saturated brine, and dried over anhydrous sodium sulfate. The solvent was evaporated under reduced pressure to give the object compound (116 mg).

MS (ESI+, m/e) 459 (M+1)

### Example 5

### Method E

### 1-[(1S)-1-(4-{[(2R)-2-Benzylpiperazin-1-yl]carbonyl}-5-phenyl-1H-imidazol-1-yl)ethyl]cyclohexanol hydrochloride

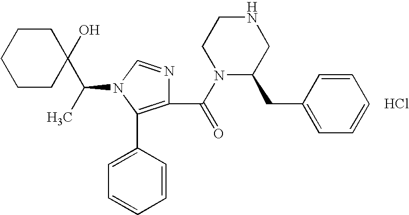

Ethyl 1-[(1S)-1-(1-hydroxycyclohexyl)ethyl]-5-phenyl-1H-imidazole-4-carboxylate (900 mg) and lithium hydroxide monohydrate (220 mg) were dissolved in a mixed solvent of methanol (10 ml) and water (2 ml). The solution was heated under reflux for 15 hr, and concentrated under reduced pressure. The residue was mixed with tert-butyl (3R)-3-benzylpiperazine-1-carboxylate (730 mg), WSC.HCl (610 mg), HOBt (1.21 g) and DMF (10 ml). The mixture was stirred at 60° C. for 3 hr, and poured into saturated aqueous sodium hydrogen carbonate, and the mixture was extracted with ethyl acetate. The extract was washed with saturated brine, and dried over anhydrous magnesium sulfate, and the solvent was evaporated under reduced pressure. The residue was subjected to silica gel column chromatography, and the object fraction was concentrated under reduced pressure to give tert-butyl (3R)-3-benzyl-4-({1-[(1S)-1-(1-hydroxycyclohexyl)ethyl]-5-phenyl-1H-imidazol-4-yl}carbonyl)piperazine-1-carboxylate. The total amount thereof was dissolved in ethyl acetate (2.5 ml), and 4N hydrogen chloride-ethyl acetate solution (2.5 ml) was added thereto. The mixture was stirred for 30 min, and concentrated under reduced pressure to give the object compound (696 mg).

MS (ESI+, m/e) 473 (M+1)

### Example 6

### Method F

### Methyl [(1S,2S)-2-(4-{[(2R)-2-benzylpiperazin-1-yl]carbonyl}-5-phenyl-1H-imidazol-1-yl)cyclohexyl]carbamate

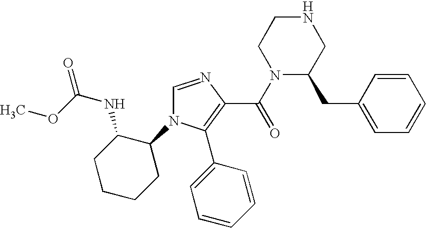

Methyl [(1S,2S)-2-(4-{[(2R)-2,4-dibenzylpiperazin-1-yl]carbonyl}-5-phenyl-1H-imidazol-1-yl)cyclohexyl]carbamate (100 mg) was dissolved in methanol (2 ml), 20% palladium hydroxide-carbon (50% containing water, 30 mg) was added thereto, and the mixture was subjected to catalytic reduction at ambient temperature and normal pressure for 15 hr. The catalyst was filtered off, and the filtrate was concentrated under reduced pressure to give the object compound (78 mg) as an amorphous solid.

MS (ESI+, m/e) 502 (M+1)

### Example 7

### Method G

### trans-2-(4-{[(2R)-2-Benzylpiperazin-1-yl]carbonyl}-5-phenyl-1H-imidazol-1-yl)cycloheptanol

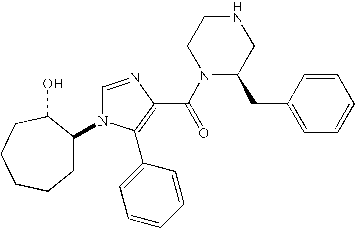

tert-Butyl (3R)-3-benzyl-4-({1-[trans-2-hydroxycycloheptyl]-5-phenyl-1H-imidazol-4-yl}carbonyl)piperazine-1-carboxylate (250 mg) was dissolved in dichloromethane (2 ml), TFA (2 ml) was added, and the mixture was stirred at room temperature for 1 hr, and concentrated under reduced pressure. The residue was dissolved in ethyl acetate, and the solution was washed successively with saturated aqueous sodium hydrogen carbonate and saturated brine, and dried over anhydrous magnesium sulfate. The solvent was evaporated under reduced pressure to give the object compound (199 mg).

MS (ESI+, m/e) 459 (M+1)

### Example 8

### Method H

### cis-2-(4-{[(2R)-2-Benzylpiperazin-1-yl]carbonyl}-5-phenyl-1H-imidazol-1-yl)cycloheptanol hydrochloride

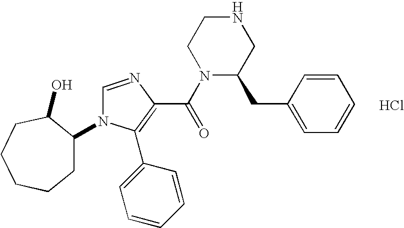

tert-Butyl (3R)-3-benzyl-4-({1-[cis-2-hydroxycycloheptyl]-5-phenyl-1H-imidazol-4-yl}carbonyl)piperazine-1-carboxylate (165 mg) was dissolved in ethyl acetate (2 ml), 4N hydrogen chloride-ethyl acetate solution (2 ml) was added, and the mixture was stirred at room temperature for 2 hr. Diethyl ether (10 ml) was added, and the crystals were collected by filtration, and washed with diethyl ether to give the object compound (146 mg).

MS (ESI+, m/e) 459 (M+1).

### Example 9

### Method I

### (1S,2R)-2-{4-[((2R)-2-{2-[(2,6-Dimethylpyridin-3-yl)oxy]ethyl}piperazin-1-yl)carbonyl]-5-phenyl-1H-imidazol-1-yl}-1-(methoxymethyl)cyclohexanol dihydrochloride

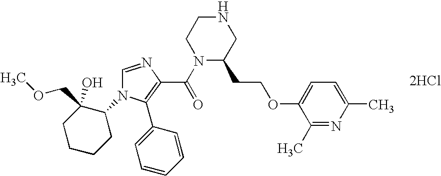

A solution of 1-[(1R,2S)-2-hydroxy-2-(methoxymethyl)cyclohexyl]-5-phenyl-1H-imidazole-4-carboxylic acid (165 mg), benzyl (3R)-3-{2-[(2,6-dimethylpyridin-3-yl)oxy]ethyl}piperazine-1-carboxylate (194 mg), WSC.HCl (115 mg) and HOBt (81 mg) in DMF (3.5 ml) was stirred at room temperature for 15 hr, and poured into saturated aqueous sodium hydrogen carbonate, and the mixture was extracted with ethyl acetate. The extract was washed successively with water and saturated brine, and dried over anhydrous magnesium sulfate, and the solvent was evaporated under reduced pressure. The residue was subjected to basic silica gel column chromatography, and the fraction eluted with ethyl acetate-hexane-methanol (1:1:0-20:0:1) was concentrated under reduced pressure to give benzyl (3R)-3-{2-[(2,6-dimethylpyridin-3-yl)oxy]ethyl}-4-({1-[(1R,2S)-2-hydroxy-2-(methoxymethyl)cyclohexyl]-5-phenyl-1H-imidazol-4-yl}carbonyl)piperazine-1-carboxylate (268 mg) as an amorphous solid. The total amount thereof was dissolved in methanol (7.5 ml), 20% palladium hydroxide-carbon (50% containing water, 135 mg) was added thereto, and the mixture was subjected to catalytic reduction at ambient temperature and normal pressure for 15 hr. The catalyst was filtered off, and the filtrate was concentrated under reduced pressure. The residue was subjected to basic silica gel column chromatography, and the fraction eluted with ethyl acetate-methanol (25:1-10:1) was concentrated under reduced pressure. The residue was diluted with diethyl ether (5 ml), 4N hydrogen chloride-ethyl acetate solution (216 μl) was added thereto, and the precipitated crystals were collected by filtration to give the object compound (184 mg).

MS (ESI+, m/e) 548 (M+1)

### Example 10

### Method J

### 1S,2R)-1-(Methoxymethyl)-2-{4-[((2R)-2-{2-[(2-methyl-1,3-benzothiazol-5-yl)oxy]ethyl}piperazin-1-yl)carbonyl]-5-phenyl-1H-imidazol-1-yl}cyclohexanol hydrochloride

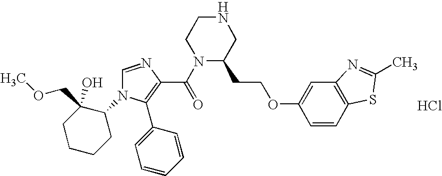

A solution of 1-[(1R,2S)-2-hydroxy-2-(methoxymethyl)cyclohexyl]-5-phenyl-1H-imidazole-4-carboxylic acid (165 mg), benzyl (3R)-3-{2-[(2-methyl-1,3-benzothiazol-5-yl)oxy]ethyl}piperazine-1-carboxylate (216 mg), WSC.HCl (115 mg) and HOBt (81 mg) in DMF (3.5 ml) was stirred at room temperature for 15 hr, and poured into saturated aqueous sodium hydrogen carbonate, and the mixture was extracted with ethyl acetate. The extract was washed successively with water and saturated brine, and dried over anhydrous magnesium sulfate, and the solvent was evaporated under reduced pressure. The residue was subjected to basic silica gel column chromatography, and the fraction eluted with ethyl acetate-hexane-methanol (1:1:0-20:0:1) was concentrated under reduced pressure to give benzyl (3R)-4-({1-[(1R,2S)-2-hydroxy-2-(methoxymethyl)cyclohexyl]-5-phenyl-1H-imidazol-4-yl}carbonyl)-3-{2-[(2-methyl-1,3-benzothiazol-5-yl)oxy]ethyl}piperazine-1-carboxylate (290 mg) as an amorphous solid. The total amount thereof was dissolved in 25% hydrogen bromide-acetic acid solution (2 ml), and the solution was stirred at room temperature for 1 hr. The reaction mixture was poured into water, and the mixture was washed with diethyl ether. Potassium carbonate was added by small portions to the aqueous layer to basify the layer, and the mixture was saturated with sodium chloride, and extracted with ethyl acetate. The extract was washed with saturated brine, and dried over anhydrous magnesium sulfate, and the solvent was evaporated under reduced pressure. The residue was subjected to basic silica gel column chromatography, and the fraction eluted with ethyl acetate-methanol (25:1-10:1) was concentrated under reduced pressure. The residue was diluted with diethyl ether (3 ml), 4N hydrogen chloride-ethyl acetate solution (110 μl) was added thereto, and the precipitated crystals were collected by filtration to give the object compound (68 mg).

MS (ESI+, m/e) 590 (M+1)

### Example 11

### Method K

### (1S,2R)-2-{4-([(2R)-2-[2-(2-Chlorophenoxy)ethyl]piperazin-1-yl}carbonyl)-5-phenyl-1H-imidazol-1-yl]-1-(methoxymethyl)cyclohexanol

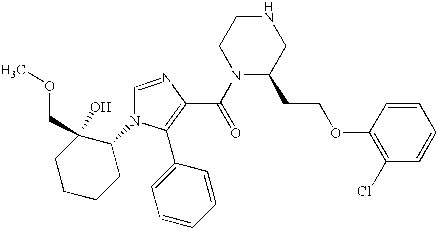

A mixture of 1-[(1R,2S)-2-hydroxy-2-(methoxymethyl)cyclohexyl]-5-phenyl-1H-imidazole-4-carboxylic acid (165 mg), benzyl (3R)-3-[2-(2-chlorophenoxy)ethyl]piperazine-1-carboxylate hydrochloride (208 mg), WSC.HCl (144 mg), HOBt (115 mg), triethylamine (101 mg) and DMF (2 ml) was stirred at room temperature for 12 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate-hexane (1:1-1:0) was concentrated under reduced pressure to give benzyl (3R)-3-[2-(2-chlorophenoxy)ethyl]-4-({1-[(1R,2S)-2-hydroxy-2-(methoxymethyl)cyclohexyl]-5-phenyl-1H-imidazol-4-yl}carbonyl)piperazine-1-carboxylate (200 mg) as an amorphous solid. The total amount thereof was dissolved in ethanol (2 ml), 4N aqueous sodium hydroxide solution (2 ml) was added, and the mixture was stirred at 65° C. for 5 hr. The reaction mixture was concentrated under reduced pressure, water was added to the residue, and the liberated oil was extracted with ethyl acetate. The extract was washed with saturated brine, and dried over anhydrous sodium sulfate, and the solvent was evaporated under reduced pressure. The residue was subjected to basic silica gel column chromatography, and the fraction eluted with ethyl acetate-hexane (1:1-1:0) was concentrated under reduced pressure to give the object compound (95 mg) as an amorphous solid.

MS (ESI+, m/e) 554 (M+1)

### Example 12

### Method L

### 1-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethyl}-1,3-dihydro-2H-benzimidazol-2-one dihydrochloride

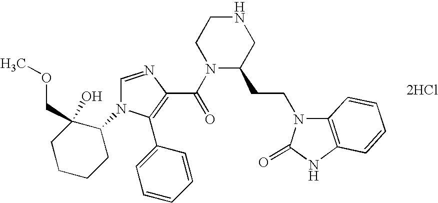

A solution of 1-[(1R,2S)-2-hydroxy-2-(methoxymethyl)cyclohexyl]-5-phenyl-1H-imidazole-4-carboxylic acid (100 mg), benzyl (3R)-3-[2-(2-oxo-2,3-dihydro-1H-benzimidazol-1-yl)ethyl]piperazine-1-carboxylate hydrochloride (125 mg), WSC.HCl (115 mg), HOBt (45 mg) and triethylamine (150 μl) in DMF (4 ml) was stirred at room temperature for 15 hr, and poured into saturated aqueous sodium hydrogen carbonate, and the mixture was extracted with ethyl acetate. The extract was washed with saturated brine, and dried over anhydrous magnesium sulfate, and the solvent was evaporated under reduced pressure. The residue was subjected to silica gel column chromatography, and the fraction eluted with ethyl acetate-methanol (1:0-17:3) was concentrated under reduced pressure to give benzyl (3R)-4-({1-[(1R,2S)-2-hydroxy-2-(methoxymethyl)cyclohexyl]-5-phenyl-1H-imidazol-4-yl}carbonyl)-3-[2-(2-oxo-2,3-dihydro-1H-benzimidazol-1-yl)ethyl]piperazine-1-carboxylate (124 mg) as an amorphous solid. The total amount thereof was dissolved in methanol (3 ml), 20% palladium hydroxide-carbon (50% containing water, 50 mg) was added thereto, and the mixture was subjected to catalytic reduction at ambient temperature and normal pressure for 10 hr. The catalyst was filtered off, and the filtrate was concentrated under reduced pressure. The residue was treated with 2N hydrogen chloride-ethyl acetate solution to give the object compound (55 mg).

MS (ESI+, m/e) 559 (M+1)

### Example 13

### Method M

### 1-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethyl}-1,2-dihydro-3H-indazol-3-one dihydrochloride

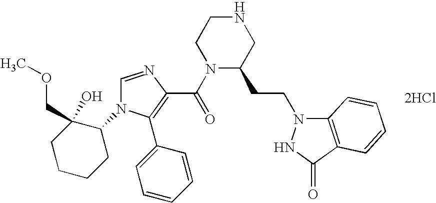

A solution of 1-[(1R,2S)-2-hydroxy-2-(methoxymethyl)cyclohexyl]-5-phenyl-1H-imidazole-4-carboxylic acid (100 mg), benzyl (3R)-3-[2-(3-oxo-2,3-dihydro-1H-indazol-1-yl)ethyl]piperazine-1-carboxylate hydrochloride (125 mg), WSC HCl (115 mg), HOBt (45 mg) and triethylamine (150 μl) in DMF (4 ml) was stirred at room temperature for 15 hr, and poured into saturated aqueous sodium hydrogen carbonate, and the mixture was extracted with ethyl acetate. The extract was washed with saturated brine, and dried over anhydrous magnesium sulfate, and the solvent was evaporated under reduced pressure. The residue was subjected to silica gel column chromatography, and the fraction eluted with ethyl acetate-methanol (1:0-17:3) was concentrated under reduced pressure to give benzyl (3R)-4-({1-[(1R,2S)-2-hydroxy-2-(methoxymethyl)cyclohexyl]-5-phenyl-1H-imidazol-4-yl}carbonyl)-3-[2-(3-oxo-2,3-dihydro-1H-indazol-1-yl)ethyl]piperazine-1-carboxylate (49 mg) as an amorphous solid. The total amount thereof was dissolved in ethanol (3 ml), 4N aqueous sodium hydroxide solution (1 ml) was added thereto, and the mixture was stirred at 70° C. for 10 hr. The reaction mixture was poured into saturated aqueous sodium hydrogen carbonate, and the mixture was extracted with ethyl acetate. The extract was washed with saturated brine, and dried over anhydrous magnesium sulfate, and the solvent was evaporated under reduced pressure. The residue was treated with 2N hydrogen chloride-ethyl acetate solution to give the object compound (16 mg).

MS (ESI+, m/e) 559 (M+1)

### Example 14

### Method N

### 1-{2-[(2R)-1-({1-[(1-Hydroxycyclohexyl)methyl]-5-phenyl-1H-imidazol-4-yl}carbonyl)piperazin-2-yl]ethyl}-1,2-dihydro-3H-indazol-3-one dihydrochloride

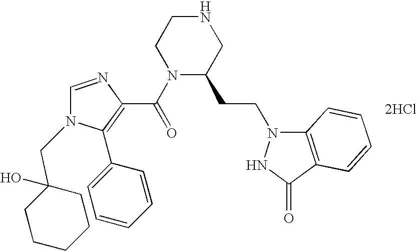

A mixture of ethyl 1-[(1-hydroxycyclohexyl)methyl]-5-phenyl-1H-imidazole-4-carboxylate (100 mg), lithium hydroxide monohydrate (20 mg), ethanol (3 ml) and water (1 ml) was stirred at 80° C. for 3 hr, and concentrated under reduced pressure. The residue was mixed with benzyl (3R)-3-[2-(3-oxo-2,3-dihydro-1H-indazol-1-yl)ethyl]piperazine-1-carboxylate hydrochloride (134 mg), WSC.HCl (115 mg), HOBt (230 mg), triethylamine (150 μl) and DMF (4 ml). The mixture was stirred at 50° C. for 5 hr, and poured into saturated aqueous sodium hydrogen carbonate, and the mixture was extracted with ethyl acetate. The extract was washed with saturated brine, and dried over anhydrous magnesium sulfate, and the solvent was evaporated under reduced pressure. The residue was subjected to silica gel column chromatography, and the fraction eluted with ethyl acetate-hexane-methanol (1:9:0-17:0:3) was concentrated under reduced pressure to give benzyl (3R)-4-({1-[(1-hydroxycyclohexyl)methyl]-5-phenyl-1H-imidazol-4-yl}carbonyl)-3-[2-(3-oxo-2,3-dihydro-1H-indazol-1-yl)ethyl]piperazine-1-carboxylate (43 mg) as an amorphous solid. The total amount thereof was dissolved in methanol (4 ml), 20% palladium hydroxide-carbon (50% containing water, 20 mg) was added thereto, and the mixture was subjected to catalytic reduction at ambient temperature and normal pressure for 12 hr. The catalyst was filtered off, and the filtrate was concentrated under reduced pressure. The residue was treated with 2N hydrogen chloride-ethyl acetate solution to give the object compound (37 mg).

MS (ESI+, m/e) 529 (M+1)

### Example 15

### Method O

### Methyl 4-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethoxy}benzoate

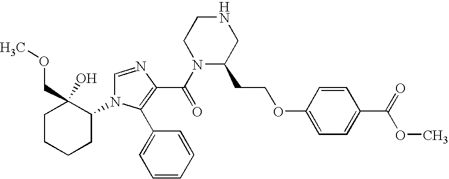

Benzyl (3R)-4-({1-[(1R,2S)-2-hydroxy-2-(methoxymethyl)cyclohexyl]-5-phenyl-1H-imidazol-4-yl}carbonyl)-3-{2-[4-(methoxycarbonyl)phenoxy]ethyl}piperazine-1-carboxylate (216 mg) was dissolved in methanol (10 ml), 20% palladium hydroxide-carbon (50% containing water, 50 mg) was added thereto, and the mixture was subjected to catalytic reduction at ambient temperature and normal pressure for 12 hr. The catalyst was filtered off, and the filtrate was concentrated under reduced pressure. The residue was suspended in ethyl acetate, and the suspension was washed with saturated aqueous sodium hydrogen carbonate, and dried over anhydrous sodium sulfate, and the solvent was evaporated under reduced pressure to give the object compound (115 mg) as an amorphous solid.

MS (ESI+, m/e) 577 (M+1)

In the same manner as in the above-mentioned Example 1 (Method A)-Example 15 (Method O), the following compounds (Examples 16-343) shown in Table 17-1-Table 17-3, Table 18-1-Table 18-10, Table 19-1-Table 19-2, Table 20-1-Table 20-2, Table 21-1-Table 21-4 and Table 22-1-Table 22-12 were obtained. Where necessary, each compound was isolated and purified by a known means such as phase transfer, liquid conversion, solvent extraction, silica gel column chromatography, reversed-phase preparative HPLC and the like. The final products were isolated as a hydrochloride by a treatment with 4N hydrogen chloride-ethyl acetate solution, as in Method A and the like, or isolated as crystals or an amorphous solid in a free from, as in Method B and the like. In the column of “Salt” in the Tables, the compounds described as “-” were isolated as a free form.

TABLE 17-1

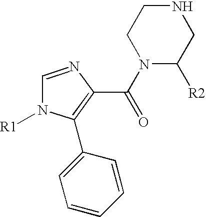

| Ex. No. | R1 | R2 | Method | Salt | MS(ESI+) |
|---|---|---|---|---|---|
| 16 | 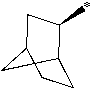 | 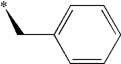 | B | — | 441 |
| 17 | 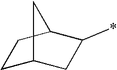 | 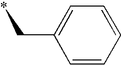 | B | — | 441 |
| 18 | 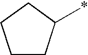 | 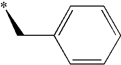 | D | — | 415 |
| 19 | 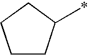 | 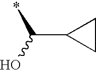 | F | HCl | 395 |
| 20 | 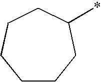 | 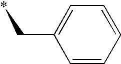 | D | — | 443 |
| 21 | 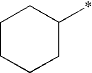 | 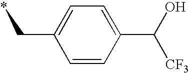 | B | — | 527 |
| 22 | 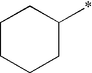 | 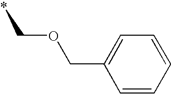 | A | 2HCl | 459 |
| 23 | 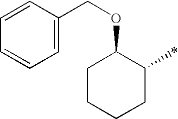 | 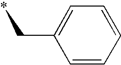 | B | — | 535 |
| 24 | 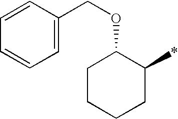 | 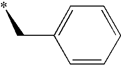 | B | — | 535 |
| 25 | 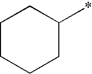 | 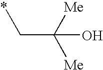 | B | — | 411 |
| 26 | 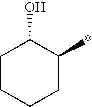 | 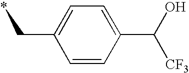 | B | — | 543 |
| 27 | 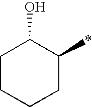 | 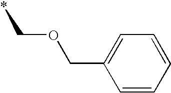 | A | — | 475 |

TABLE 17-2

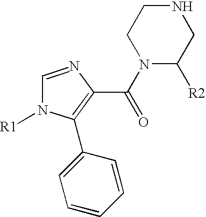

| Ex. No. | R1 | R2 | Method | Salt | MS(ESI+) |
|---|---|---|---|---|---|
| 28 | 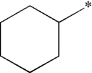 | 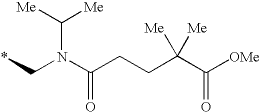 | A | HCl | 566 |
| 29 | 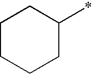 | 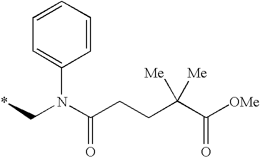 | A | HCl | 600 |
| 30 | 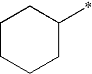 | 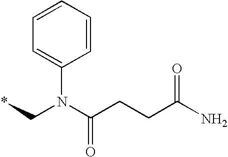 | A | HCl | 543 |
| 31 | 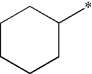 | 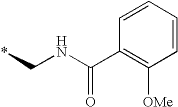 | A | HCl | 502 |
| 32 | 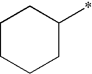 | 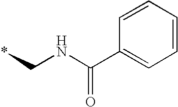 | A | HCl | 472 |
| 33 | 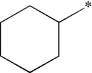 | 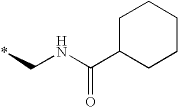 | A | HCl | 478 |
| 34 | 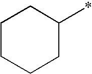 | 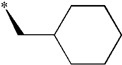 | A | — | 435 |
| 35 | 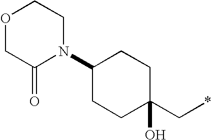 | 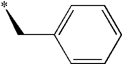 | D | TFA | 558 |
| 36 | 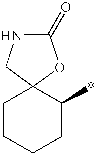 | 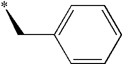 | G | — | 500 |

TABLE 17-3

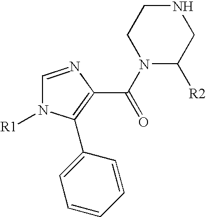

| Ex. No. | R1 | R2 | Method | Salt | MS(ESI+) |
|---|---|---|---|---|---|
| 37 | 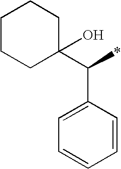 | 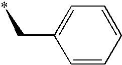 | E | HCl | 535 |
| 38 | 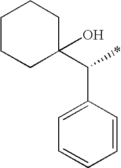 | 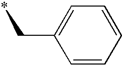 | E | — | 535 |
| 39 | 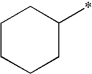 | 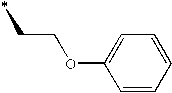 | K | 2HCl | 459 |
| 40 | 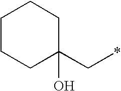 | 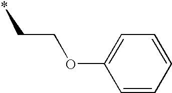 | N | 2HCl | 489 |
| 41 | 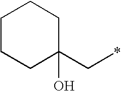 | 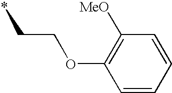 | L | — | 519 |

TABLE 18-1

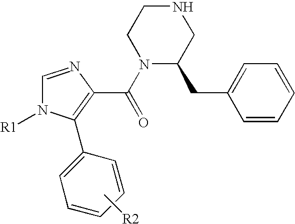

| Ex. | | | | | MS |
|---|---|---|---|---|---|
| No. | R1 | R2 | Method | Salt | (ESI+) |
| 42 | 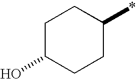 | H | E | HCl | 445 |
| 43 | 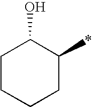 | H | G | — | 445 |
| 44 | 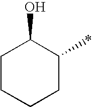 | H | G | — | 445 |
| 45 | 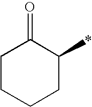 | H | H | HCl | 443 |
| 46 | 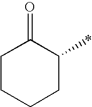 | H | H | HCl | 443 |
| 47 | 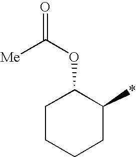 | H | G | HCl | 487 |
| 48 | 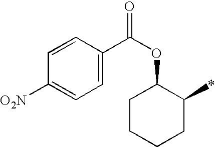 | H | G | HCl | 594 |
| 49 | 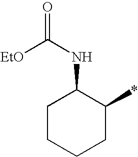 | H | B | — | 516 |
| 50 | 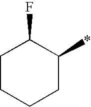 | H | B | — | 447 |
| 51 | 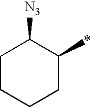 | H | G | — | 470 |

TABLE 18-2

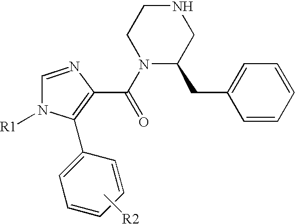

| Ex. No. | R1 | R2 | Method | Salt | MS(ESI+) |
|---|---|---|---|---|---|
| 52 | 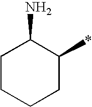 | H | H | 2HCl | 444 |
| 53 | 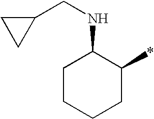 | H | H | 2HCl | 571 |
| 54 | 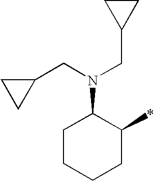 | H | H | 2HCl | 652 |
| 55 | 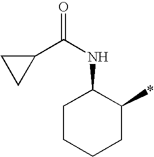 | H | G | — | 512 |
| 56 | 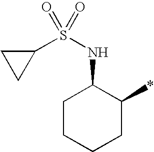 | H | G | — | 548 |
| 57 | 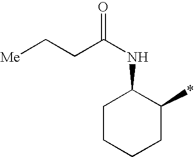 | H | G | — | 514 |
| 58 | 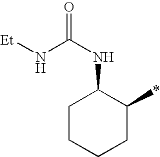 | H | G | — | 515 |
| 59 | 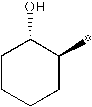 | 2,3-F₂ | B | — | 481 |
| 60 | 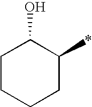 | 3-F | G | — | 463 |

TABLE 18-3

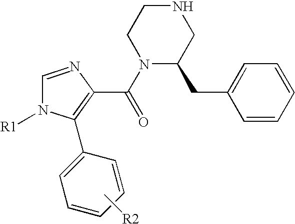

| Ex. No. | R1 | R2 | Method | Salt | MS(ESI+) |
|---|---|---|---|---|---|
| 61 | 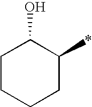 | 4-F | B | — | 463 |
| 62 | 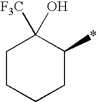 | H | H | HCl | 513 |
| 63 | 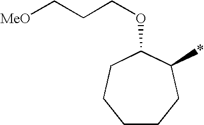 | H | H | HCl | 531 |
| 64 | 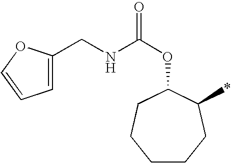 | H | H | HCl | 582 |
| 65 | 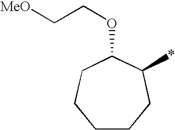 | H | H | HCl | 517 |
| 66 | 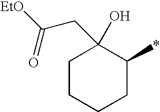 | H | G | — | 531 |
| 67 | 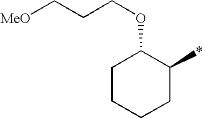 | H | H | HCl | 517 |
| 68 | 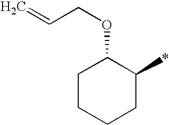 | H | H | HCl | 485 |
| 69 | 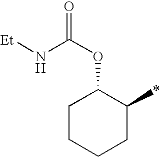 | H | H | — | 516 |

TABLE 18-4

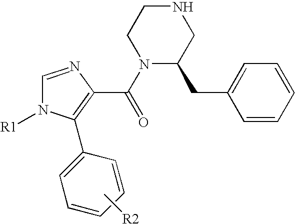

| Ex. No. | R1 | R2 | Method | Salt | MS(ESI+) |
|---|---|---|---|---|---|
| 70 | 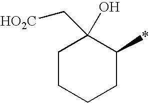 | H | G | TFA | 503 |
| 71 | 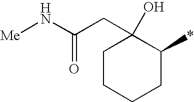 | H | G | — | 516 |
| 72 | 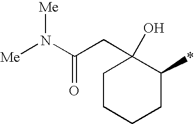 | H | G | — | 530 |
| 73 | 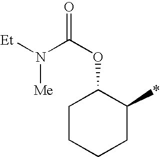 | H | H | HCl | 530 |
| 74 | 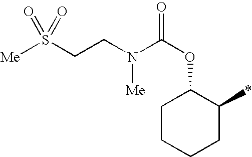 | H | H | HCl | 608 |
| 75 | 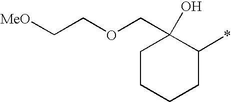 | H | H | HCl | 533 |
| 76 | 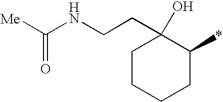 | H | G | TFA | 530 |
| 77 | 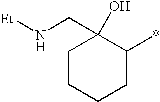 | H | H | 2HCl | 502 |
| 78 | 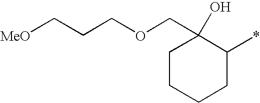 | H | H | HCl | 547 |
| 79 | 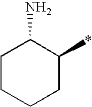 | H | F | — | 444 |

TABLE 18-5

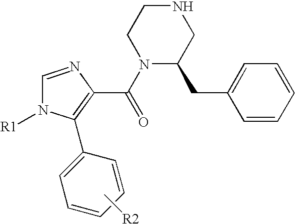

| Ex. No. | R1 | R2 | Method | Salt | MS(ESI+) |
|---|---|---|---|---|---|
| 80 | 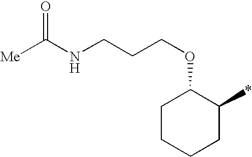 | H | G | — | 544 |
| 81 | 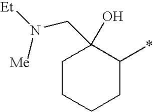 | H | H | 2HCl | 516 |
| 82 | 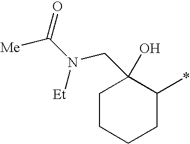 | H | H | — | 544 |
| 83 | 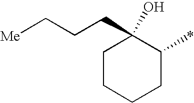 | H | H | HCl | 501 |
| 84 | 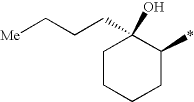 | H | H | HCl | 501 |
| 85 | 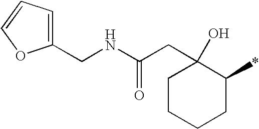 | H | G | — | 582 |
| 86 | 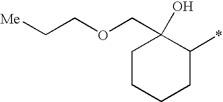 | H | H | HCl | 517 |
| 87 | 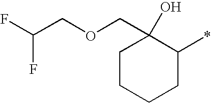 | H | H | HCl | 539 |
| 88 | 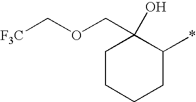 | H | H | HCl | 557 |
| 89 | 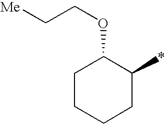 | H | H | HCl | 487 |

TABLE 18-6

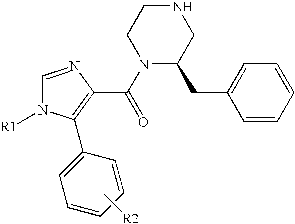

| Ex. No. | R1 | R2 | Method | Salt | MS(ESI+) |
|---|---|---|---|---|---|
| 90 | 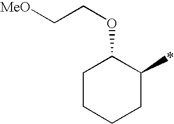 | H | H | HCl | 503 |
| 91 | 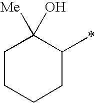 | H | H | HCl | 459 |
| 92 | 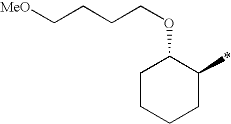 | H | H | HCl | 531 |
| 93 | 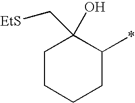 | H | H | HCl | 519 |
| 94 | 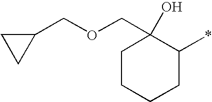 | H | H | HCl | 529 |
| 95 | 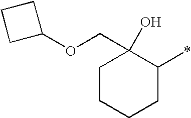 | H | H | HCl | 529 |
| 96 | 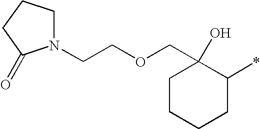 | H | H | HCl | 586 |
| 97 | 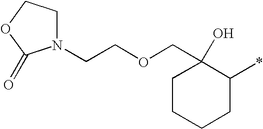 | H | H | HCl | 588 |
| 98 | 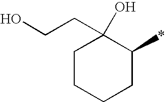 | H | G | HCl | 489 |
| 99 | 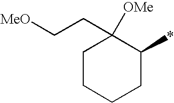 | H | G | HCl | 517 |

TABLE 18-7

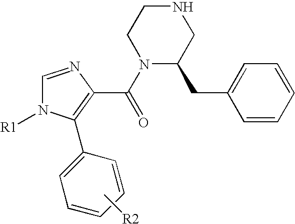

| Ex. No. | R1 | R2 | Method | Salt | MS(ESI+) |
|---|---|---|---|---|---|
| 100 | 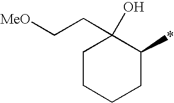 | H | G | HCl | 503 |
| 101 | 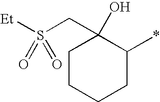 | H | H | HCl | 551 |
| 102 | 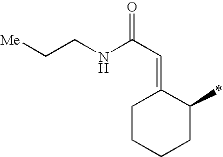 | H | H | HCl | 526 |
| 103 | 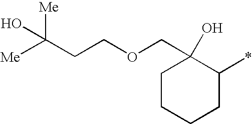 | H | H | HCl | 561 |
| 104 | 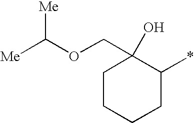 | H | H | HCl | 517 |
| 105 | 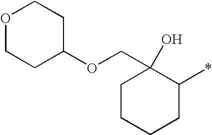 | H | H | HCl | 559 |
| 106 | 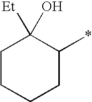 | H | H | HCl | 473 |
| 107 | 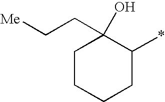 | H | H | HCl | 487 |
| 108 | 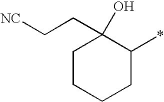 | H | G | — | 498 |
| 109 | 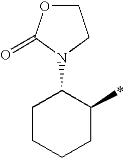 | H | F | — | 514 |

TABLE 18-8

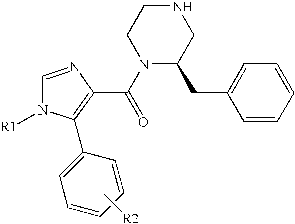

| Ex. No. | R1 | R2 | Method | Salt | MS(ESI+) |
|---|---|---|---|---|---|
| 110 | 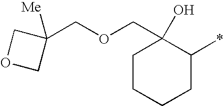 | H | G | — | 559 |
| 111 | 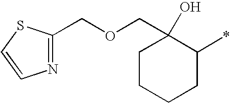 | H | H | HCl | 572 |
| 112 | 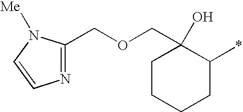 | H | H | 2HCl | 569 |
| 113 | 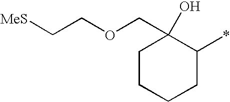 | H | H | HCl | 549 |
| 114 | 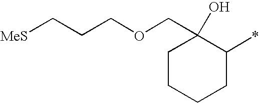 | H | H | HCl | 563 |
| 115 | 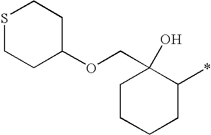 | H | H | HCl | 575 |
| 116 | 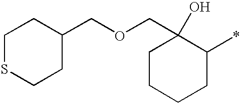 | H | H | HCl | 589 |
| 117 | 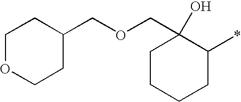 | H | H | HCl | 573 |
| 118 | 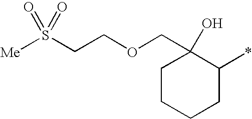 | H | H | HCl | 581 |
| 119 | 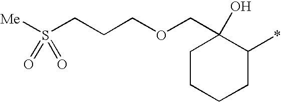 | H | H | HCl | 595 |
| 120 | 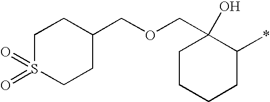 | H | H | HCl | 621 |

TABLE 18-9

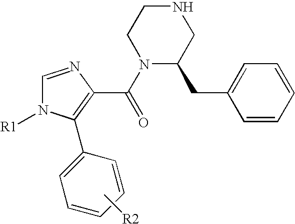

| Ex. No. | R1 | R2 | Method | Salt | MS(ESI+) |
|---|---|---|---|---|---|
| 121 | 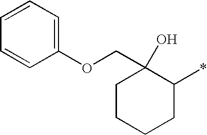 | H | H | HCl | 551 |
| 122 | 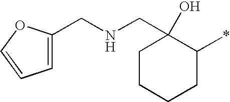 | H | H | HCl | 554 |
| 123 | 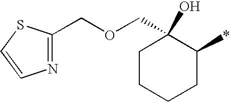 | H | H | HCl | 572 |
| 124 | 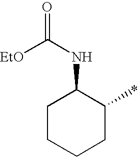 | H | D | — | 516 |
| 125 | 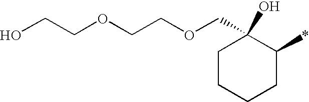 | H | H | — | 563 |
| 126 | 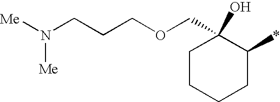 | H | H | HCl | 560 |
| 127 | 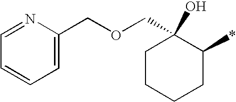 | H | H | 2HCl | 566 |
| 128 | 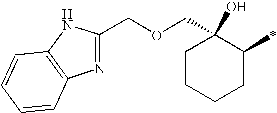 | H | H | — | 605 |
| 129 | 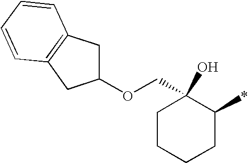 | H | H | HCl | 591 |
| 130 | 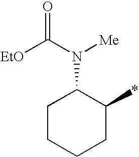 | H | G | — | 530 |

TABLE 18-10

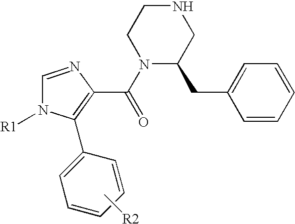

| Ex. | | | | | MS |
|---|---|---|---|---|---|
| No. | R1 | R2 | Method | Salt | (ESI+) |
| 131 | 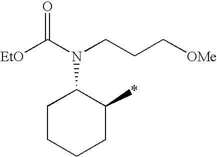 | H | G | — | 588 |
| 132 | 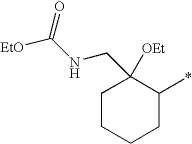 | H | D | — | 574 |

TABLE 19-1

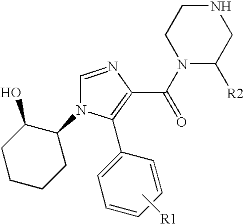

| Ex. | | | | | |
|---|---|---|---|---|---|
| No. | R1 | R2 | Method | Salt | MS(ESI+) |
| 133 | H | 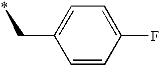 | A | HCl | 463 |
| 134 | H | 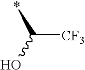 | A | — | 453 |
| 135 | 2-F | 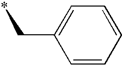 | B | — | 463 |
| 136 | 3,5-F₂ | 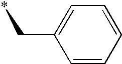 | B | — | 481 |
| 137 | H | 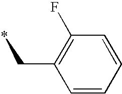 | A | HCl | 463 |
| 138 | H | 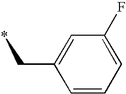 | A | HCl | 463 |
| 139 | H | 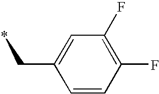 | A | HCl | 481 |
| 140 | H | 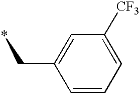 | A | HCl | 513 |
| 141 | H | 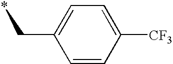 | A | HCl | 513 |
| 142 | H | 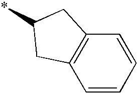 | A | HCl | 471 |
| 143 | H | 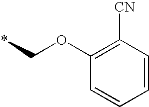 | B | — | 486 |
| 144 | H | 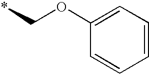 | D | — | 461 |
| 145 | H | 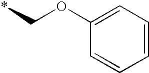 | E | HCl | 461 |

TABLE 19-2

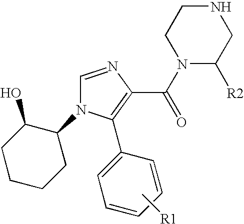

| Ex. No. | R1 | R2 | Method | Salt | MS(ESI+) |
|---|---|---|---|---|---|
| 146 | H | 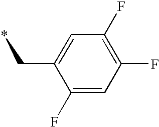 | A | HCl | 499 |
| 147 | H | 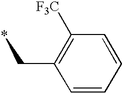 | A | HCl | 513 |
| 148 | H | 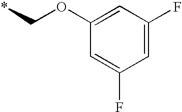 | E | HCl | 497 |
| 149 | H | 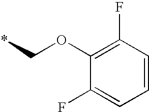 | E | HCl | 497 |
| 150 | H | 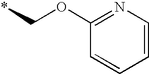 | E | 2HCl | 462 |
| 151 | H | 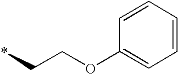 | L | — | 475 |

TABLE 20-1

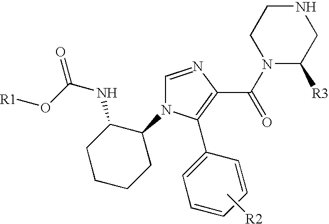

| Ex. No. | R1 | R2 | R3 | Method | Salt | MS(ESI+) |
|---|---|---|---|---|---|---|
| 152 | 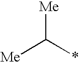 | H | 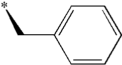 | F | — | 530 |
| 153 | 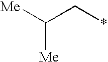 | H | 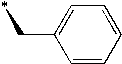 | F | — | 544 |
| 154 | 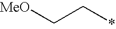 | H | 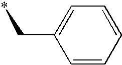 | F | — | 546 |
| 155 | Et | H | 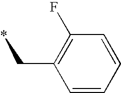 | A | — | 534 |
| 156 | Et | H | 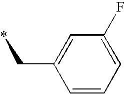 | A | — | 534 |
| 157 | Et | H | 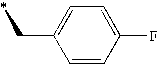 | A | — | 534 |
| 158 | Et | H | 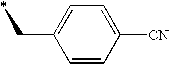 | B | — | 541 |
| 159 | Me | H | 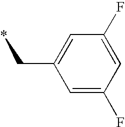 | A | — | 538 |
| 160 | Me | H | 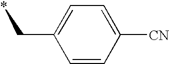 | B | — | 527 |
| 161 | Me | 3-F | 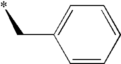 | D | — | 520 |
| 162 | Et | 3-F | 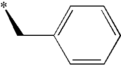 | D | — | 534 |
| 163 | Me | H | 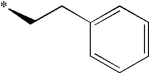 | A | — | 516 |
| 164 | Me | H | 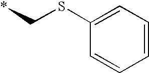 | B | — | 534 |
| 165 | Me | H | 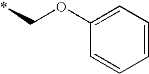 | B | — | 518 |

TABLE 20-2

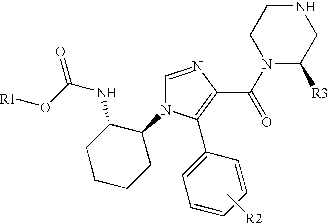

| Ex. No. | R1 | R2 | R3 | Method | Salt | MS(ESI+) |
|---|---|---|---|---|---|---|
| 166 | Me | H | 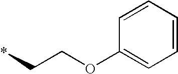 | I | — | 532 |
| 167 | Me | H | 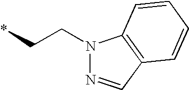 | I | — | 556 |
| 168 | Et | H | 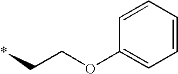 | I | — | 546 |

TABLE 21-1

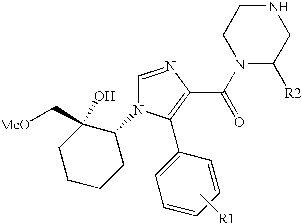

| Ex. No. | R1 | R2 | Method | Salt | MS(ESI+) |
|---|---|---|---|---|---|
| 169 | H | 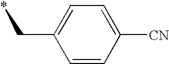 | C | HCl | 513 |
| 170 | H | 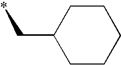 | A | HCl | 495 |
| 171 | H | 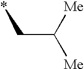 | A | HCl | 455 |
| 172 | H | 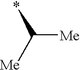 | A | HCl | 441 |
| 173 | H | 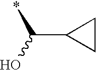 | A | HCl | 469 |
| 174 | H | 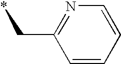 | A | — | 490 |
| 175 | H | 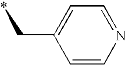 | A | 2HCl | 490 |
| 176 | 3-F | 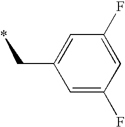 | A | — | 543 |
| 177 | 3-F | 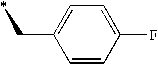 | A | — | 525 |
| 178 | 3-F | 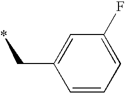 | A | — | 525 |
| 179 | H | 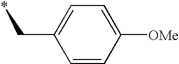 | A | HCl | 519 |
| 180 | H | 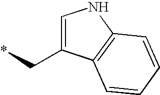 | A | HCl | 528 |
| 181 | 3-F | 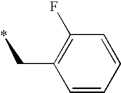 | A | — | 525 |
| 182 | 3-F | 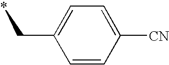 | B | — | 532 |

TABLE 21-2

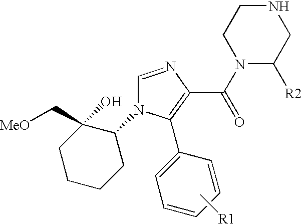

| Ex. No. | R1 | R2 | Method | Salt | MS(ESI+) |
|---|---|---|---|---|---|
| 183 | H | 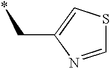 | B | 2HCl | 496 |
| 184 | H | 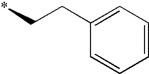 | A | HCl | 503 |
| 185 | H | 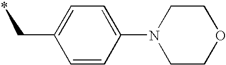 | A | 2HCl | 574 |
| 186 | H | 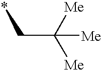 | A | HCl | 469 |
| 187 | H | 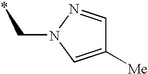 | B | — | 493 |
| 188 | H | 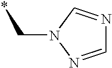 | C | — | 480 |
| 189 | H | 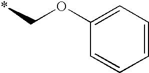 | C | HCl | 505 |
| 190 | 3-F | 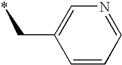 | A | — | 508 |
| 191 | H | 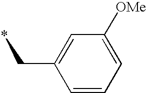 | C | HCl | 519 |
| 192 | H | 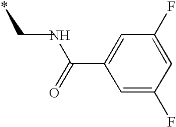 | A | HCl | 568 |
| 193 | H | 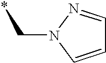 | C | HCl | 479 |
| 194 | H | 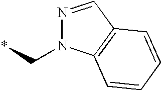 | C | HCl | 529 |
| 195 | H | 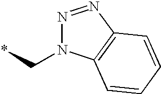 | C | — | 530 |

TABLE 21-3

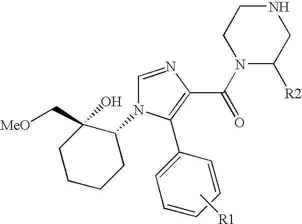

| Ex. No. | R1 | R2 | Method | Salt | MS(ESI+) |
|---|---|---|---|---|---|
| 196 | H | 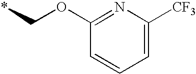 | B | — | 574 |
| 197 | H | 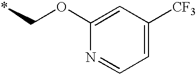 | B | — | 574 |
| 198 | H | 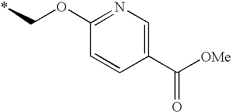 | C | 3HCl | 564 |
| 199 | H | 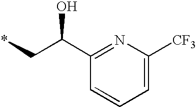 | H | 3HCl | 588 |
| 200 | H | 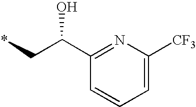 | H | 3HCl | 588 |
| 201 | H | 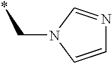 | C | 3HCl | 479 |
| 202 | H | 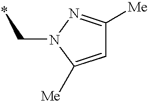 | C | 2HCl | 507 |
| 203 | H | 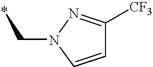 | C | 2HCl | 547 |
| 204 | H | 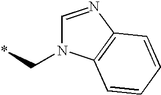 | C | — | 529 |
| 205 | H | 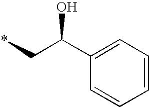 | H | 2HCl | 519 |
| 206 | H | 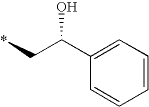 | H | 2HCl | 519 |
| 207 | H | 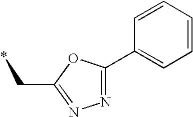 | A | HCl | 557 |

TABLE 21-4

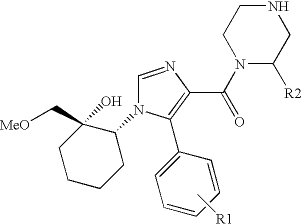

| Ex. No. | R1 | R2 | Method | Salt | MS(ESI+) |
|---|---|---|---|---|---|
| 208 | H | 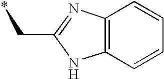 | A | — | 529 |
| 209 | H | 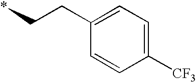 | B | — | 571 |
| 210 | 3-F | 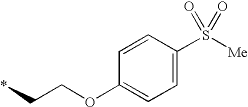 | L | — | 615 |
| 211 | H | 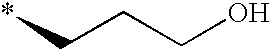 | I | — | 457 |
| 212 | H | 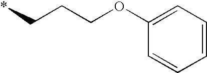 | I | — | 533 |
| 213 | H | 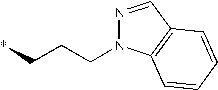 | I | — | 557 |
| 214 | H | 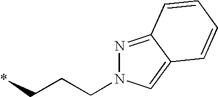 | I | — | 557 |
| 215 | H | 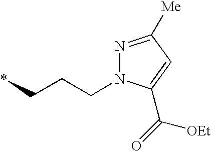 | I | — | 593 |
| 216 | H | 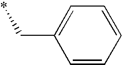 | A | — | 489 |
| 217 | 3-F | 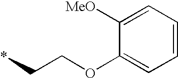 | L | — | 567 |

TABLE 22-1

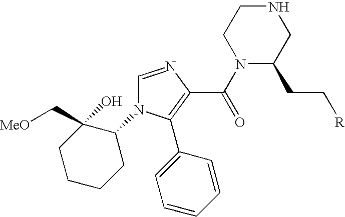

| Ex. No. | R | Method | Salt | MS(ESI+) |
|---|---|---|---|---|
| 218 | OH | F | — | 443 |
| 219 | 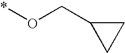 | A | — | 497 |
| 220 | 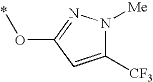 | I | 2HCl | 591 |
| 221 | 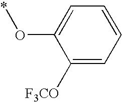 | I | 2HCl | 603 |
| 222 | 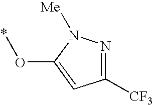 | I | 2HCl | 591 |
| 223 | 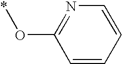 | H | 3HCl | 520 |
| 224 | 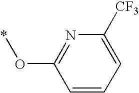 | H | 3HCl | 588 |
| 225 | 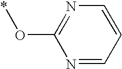 | H | 2HCl | 521 |
| 226 | 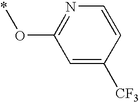 | H | 3HCl | 588 |
| 227 | 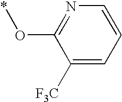 | H | 3HCl | 588 |
| 228 | 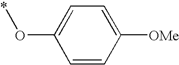 | L | — | 549 |
| 229 | 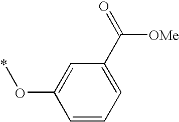 | O | — | 577 |
| 230 | 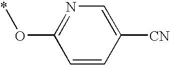 | H | 3HCl | 545 |

TABLE 22-2

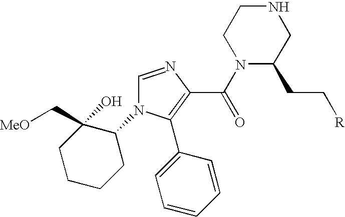

| Ex. No. | R | Method | Salt | MS(ESI+) |
|---|---|---|---|---|
| 231 | 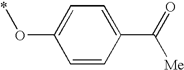 | I | — | 561 |
| 232 | 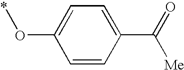 | K | 2HCl | 561 |
| 233 | 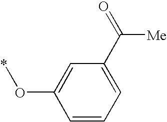 | I | — | 561 |
| 234 | 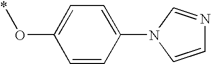 | I | — | 585 |
| 235 | 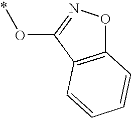 | I | 2HCl | 560 |
| 236 | 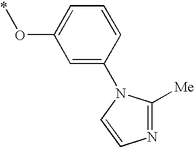 | I | — | 599 |
| 237 | 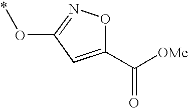 | J | 2TFA | 568 |
| 238 | 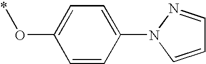 | L | — | 585 |
| 239 | 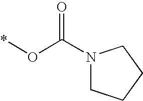 | H | 2HCl | 540 |
| 240 | 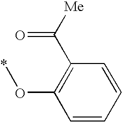 | M | — | 561 |
| 241 | 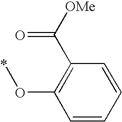 | O | — | 577 |

TABLE 22-3

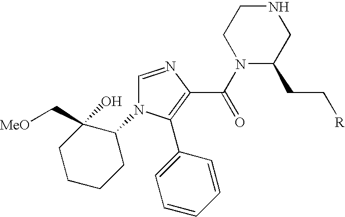

| Ex. No. | R | Method | Salt | MS(ESI+) |
|---|---|---|---|---|
| 242 | 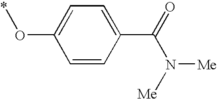 | O | — | 590 |
| 243 | 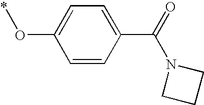 | O | — | 602 |
| 244 | 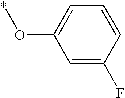 | I | — | 537 |
| 245 | 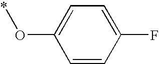 | I | — | 537 |
| 246 | 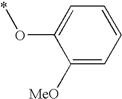 | I | — | 549 |
| 247 | 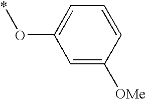 | I | — | 549 |
| 248 | 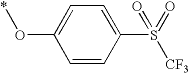 | L | — | 651 |
| 249 | 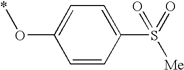 | L | — | 597 |
| 250 | 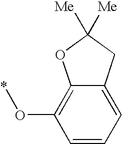 | I | HCl | 589 |
| 251 | 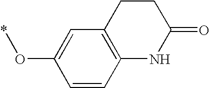 | I | — | 588 |
| 252 | 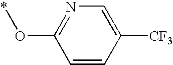 | H | 3HCl | 588 |
| 253 | 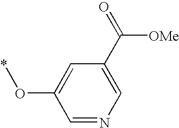 | I | 2HCl | 578 |

TABLE 22-4

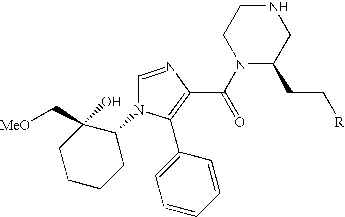

| Ex. | | | | MS |
|---|---|---|---|---|
| No. | R | Method | Salt | (ESI+) |
| 254 | 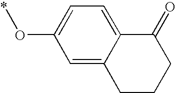 | I | HCl | 587 |
| 255 | 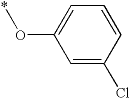 | K | — | 554 |
| 256 | 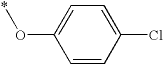 | K | — | 554 |
| 257 | 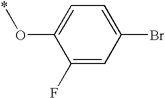 | K | — | 616 |
| 258 | 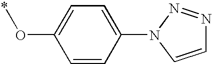 | K | 2HCl | 586 |
| 259 | 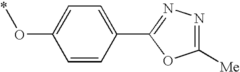 | K | 2HCl | 601 |
| 260 | 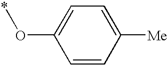 | I | — | 533 |
| 261 | 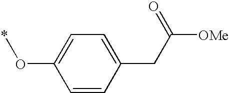 | I | — | 591 |
| 262 | 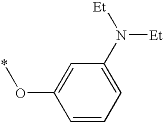 | K | 2HCl | 590 |
| 263 | 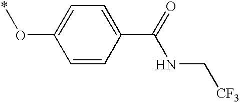 | O | — | 644 |
| 264 | 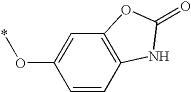 | I | — | 576 |
| 265 | 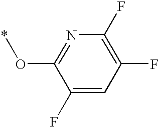 | I | 2HCl | 574 |

TABLE 22-5

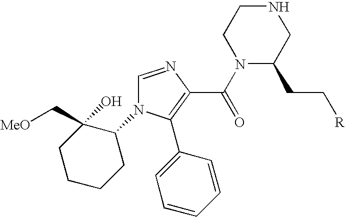

| Ex. No. | R | Method | Salt | MS(ESI+) |
|---|---|---|---|---|
| 266 | 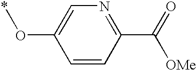 | I | 2HCl | 578 |
| 267 | 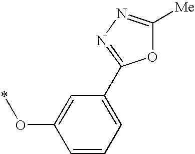 | K | 2HCl | 601 |
| 268 | 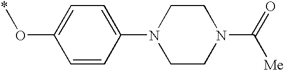 | O | 2HCl | 645 |
| 269 | 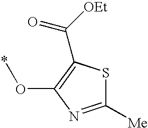 | J | HCl | 612 |
| 270 | 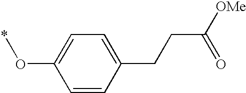 | I | — | 605 |
| 271 | 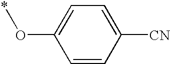 | J | — | 544 |
| 272 | 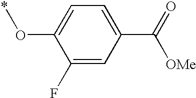 | I | — | 595 |
| 273 | 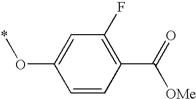 | I | — | 595 |
| 274 | 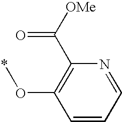 | I | 2HCl | 578 |
| 275 | 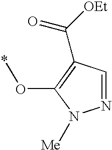 | I | HCl | 595 |

TABLE 22-6

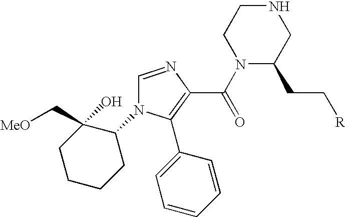

| Ex. No. | R | Method | Salt | MS(ESI+) |
|---|---|---|---|---|
| 276 | 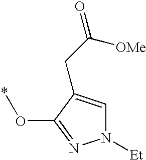 | I | HCl | 609 |
| 277 | 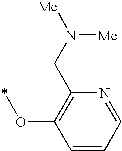 | J | 3HCl | 577 |
| 278 | 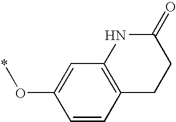 | I | — | 588 |
| 279 | 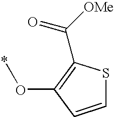 | J | HCl | 583 |
| 280 | 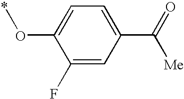 | K | — | 579 |
| 281 | 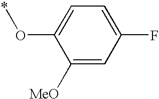 | I | — | 567 |
| 282 | 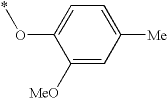 | K | 2HCl | 563 |
| 283 | 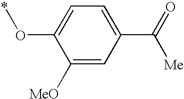 | K | 2HCl | 591 |
| 284 | 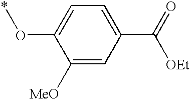 | I | 2HCl | 621 |
| 285 | 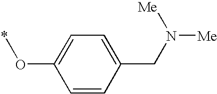 | K | — | 576 |

TABLE 22-7

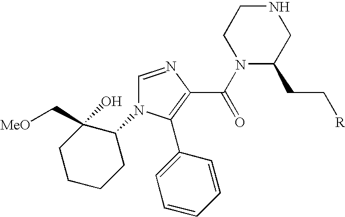

| Ex. No. | R | Method | Salt | MS(ESI+) |
|---|---|---|---|---|
| 286 | 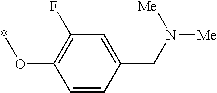 | K | — | 594 |
| 287 | 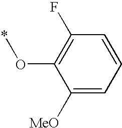 | I | — | 567 |
| 288 | 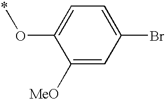 | J | HCl | 627 |
| 289 | 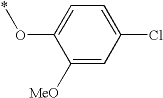 | J | HCl | 583 |
| 290 | 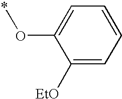 | I | HCl | 563 |
| 291 | 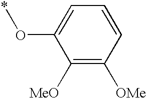 | I | HCl | 579 |
| 292 | 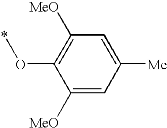 | I | HCl | 593 |
| 293 | 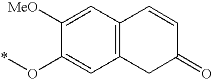 | I | — | 617 |
| 294 | 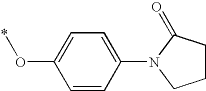 | I | — | 602 |
| 295 | 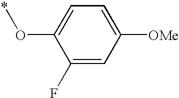 | I | — | 567 |
| 296 | 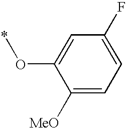 | I | — | 567 |

TABLE 22-8

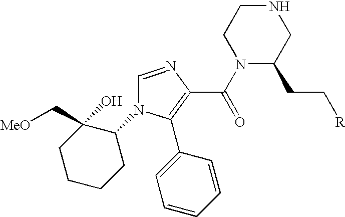

| Ex. | | | | MS |
|---|---|---|---|---|
| No. | R | Method | Salt | (ESI+) |
| 297 | 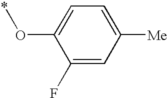 | I | — | 551 |
| 298 | 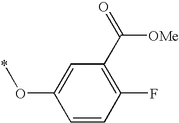 | I | — | 595 |
| 299 | 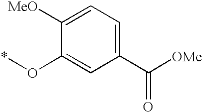 | L | — | 607 |
| 300 | 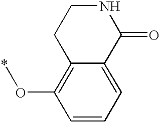 | I | — | 588 |
| 301 | 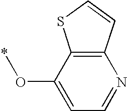 | J | 2HCl | 576 |
| 302 | 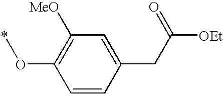 | I | 2HCl | 635 |
| 303 | 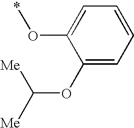 | I | HCl | 577 |
| 304 | 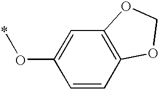 | I | HCl | 563 |
| 305 | 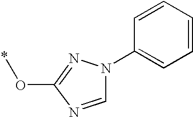 | I | HCl | 586 |
| 306 | 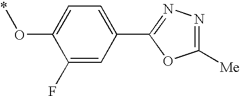 | L | — | 619 |

TABLE 22-9

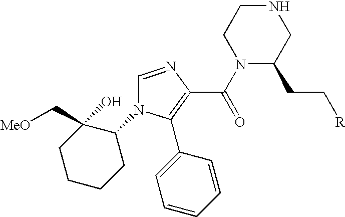

| Ex. | | | | MS |
|---|---|---|---|---|
| No. | R | Method | Salt | (ESI+) |
| 307 | 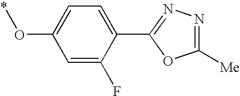 | L | — | 619 |
| 308 | 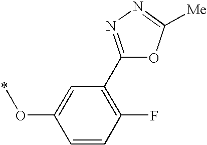 | L | — | 619 |
| 309 | 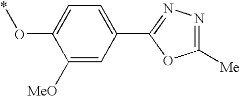 | L | — | 631 |
| 310 | 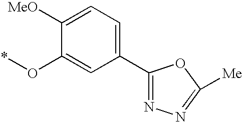 | L | — | 631 |
| 311 | 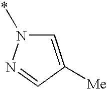 | I | 2HCl | 507 |
| 312 | 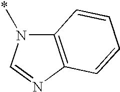 | I | — | 543 |
| 313 | 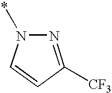 | I | — | 561 |
| 314 | 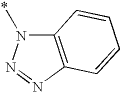 | I | — | 544 |
| 315 | 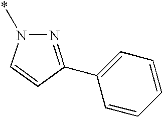 | I | — | 569 |
| 316 | 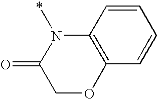 | M | 2HCl | 574 |
| 317 | 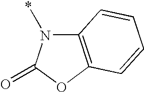 | L | 2HCl | 560 |

TABLE 22-10

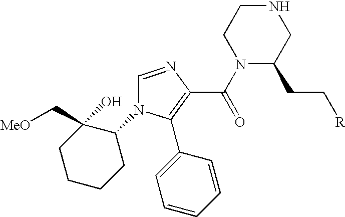

| Ex. No. | R | Method | Salt | MS(ESI+) |
|---|---|---|---|---|
| 318 | 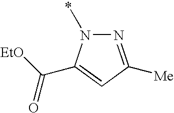 | I | — | 579 |
| 319 | 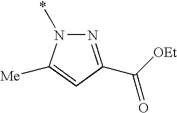 | I | — | 579 |
| 320 | 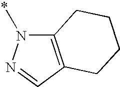 | I | — | 681 |
| 321 | 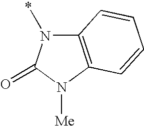 | L | — | 573 |
| 322 | 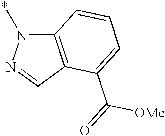 | I | — | 601 |
| 323 | 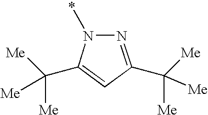 | I | — | 605 |
| 324 | 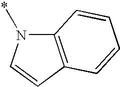 | L | — | 542 |
| 325 | 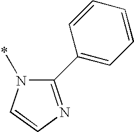 | L | — | 569 |
| 326 | 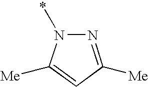 | I | — | 521 |
| 327 | 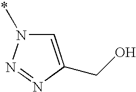 | L | — | 524 |
| 328 | 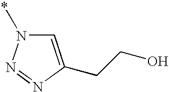 | L | — | 538 |

TABLE 22-11

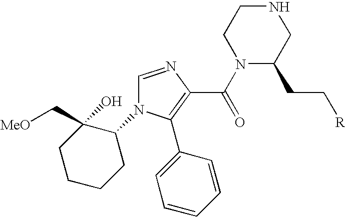

| Ex. No. | R | Method | Salt | MS(ESI+) |
|---|---|---|---|---|
| 329 | 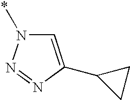 | L | — | 534 |
| 330 | 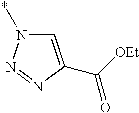 | L | — | 566 |
| 331 | 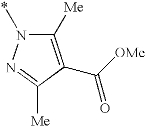 | I | — | 579 |
| 332 | 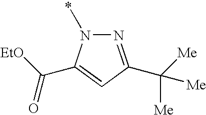 | I | — | 621 |
| 333 | 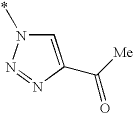 | L | — | 536 |
| 334 | 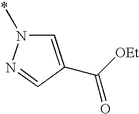 | I | — | 565 |
| 335 | 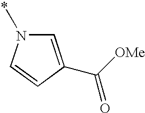 | L | — | 550 |
| 336 | 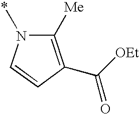 | L | — | 578 |
| 337 | 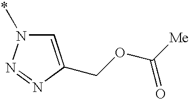 | L | 2HCl | 566 |
| 338 | 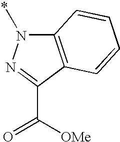 | I | — | 601 |

TABLE 22-12

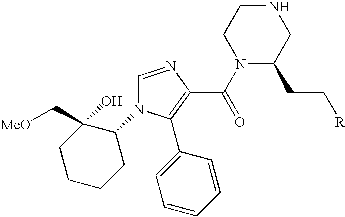

| Ex. No. | R | Method | Salt | MS(ESI+) |
|---|---|---|---|---|
| 339 | 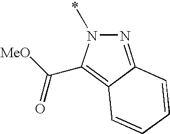 | I | — | 601 |
| 340 | 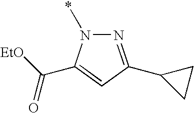 | I | — | 605 |
| 341 | 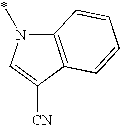 | J | — | 567 |
| 342 | 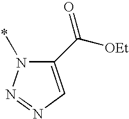 | L | — | 566 |
| 343 | 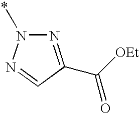 | L | — | 566 |

The chemical names of the compounds (Examples 16-343) shown in Table 17-1-Table 17-3, Table 18-1-Table 18-10, Table 19-1-Table 19-2, Table 20-1-Table 20-2, Table 21-1-Table 21-4 and Table 22-1-Table 22-12 are as follows.
Example 16: (2R)-2-Benzyl-1-({1-[(2R)-bicyclo[2.2.1]hept-2-yl]-phenyl-1H-imidazol-4-yl}carbonyl)piperazine
Example 17: (2R)-2-Benzyl-1-{[1-(bicyclo[2.2.1]hept-2-yl)-5-phenyl-1H-imidazol-4-yl]carbonyl}piperazine
Example 18: (2R)-2-Benzyl-1-[(1-cyclopentyl-5-phenyl-1H-imidazol-4-yl)carbonyl]piperazine
Example 19: {(2S)-1-[(1-Cyclopentyl-5-phenyl-1H-imidazol-4-yl)carbonyl]piperazin-2-yl}(cyclopropyl)methanol hydrochloride
Example 20: (2R)-2-Benzyl-1-[(1-cycloheptyl-5-phenyl-1H-imidazol-4-yl)carbonyl]piperazine
Example 21: 1-[4-({(2R)-1-[(1-Cyclohexyl-5-phenyl-1H-imidazol-4-yl)carbonyl]piperazin-2-yl}methyl)phenyl]-2,2,2-trifluoroethanol
Example 22: (2S)-2-[(Benzyloxy)methyl]-1-[(1-cyclohexyl-5-phenyl-1H-imidazol-4-yl)carbonyl]piperazine dihydrochloride
Example 23: (2R)-2-Benzyl-1-({1-[(1R,2R)-2-(benzyloxy)cyclohexyl]-5-phenyl-1H-imidazol-4-yl}carbonyl)piperazine
Example 24: (2R)-2-Benzyl-1-({1-[(1S,2S)-2-(benzyloxy)cyclohexyl]-5-phenyl-1H-imidazol-4-yl}carbonyl)piperazine
Example 25: 1-{1-[(1-Cyclohexyl-5-phenyl-1H-imidazol-4-yl)carbonyl]piperazin-2-yl}-2-methylpropan-2-ol
Example 26: (1S,2S)-2-[5-Phenyl-4-({(2R)-2-[4-(2,2,2-trifluoro-1-hydroxyethyl)benzyl]piperazin-1-yl}carbonyl)-1H-imidazol-1-yl]cyclohexanol
Example 27: (1S,2S)-2-[4-({(2S)-2-[(Benzyloxy)methyl]piperazin-1-yl}carbonyl)-5-phenyl-1H-imidazol-1-yl]cyclohexanol
Example 28: Methyl 5-[({(2S)-1-[(1-cyclohexyl-5-phenyl-1H-imidazol-4-yl)carbonyl]piperazin-2-yl}methyl)(isopropyl)amino]-2,2-dimethyl-5-oxopentanoate hydrochloride
Example 29: Methyl 5-[({(2S)-1-[(1-cyclohexyl-5-phenyl-1H-imidazol-4-yl)carbonyl]piperazin-2-yl}methyl)(phenyl)amino]-2,2-dimethyl-5-oxopentanoate hydrochloride
Example 30: N-({(2S)-1-[(1-Cyclohexyl-5-phenyl-1H-imidazol-4-yl)carbonyl]piperazin-2-yl}methyl)-N-phenylsuccinamide hydrochloride
Example 31: N-({(2S)-1-[(1-Cyclohexyl-5-phenyl-1H-imidazol-4-yl)carbonyl]piperazin-2-yl}methyl)-2-methoxybenzamide hydrochloride
Example 32: N-({(2S)-1-[(1-Cyclohexyl-5-phenyl-1H-imidazol-4-yl)carbonyl]piperazin-2-yl}methyl)benzamide hydrochloride
Example 33: N-({(2S)-1-[(1-Cyclohexyl-5-phenyl-1H-imidazol-4-yl)carbonyl]piperazin-2-yl}methyl)cyclohexanecarboxamide hydrochloride
Example 34: (2R)-2-(Cyclohexylmethyl)-1-[(1-cyclohexyl-5-phenyl-1H-imidazol-4-yl)carbonyl]piperazine
Example 35: 4-{cis-4-[(4-{[(2R)-2-Benzylpiperazin-1-yl]carbonyl}-5-phenyl-1H-imidazol-1-yl)methyl]-4-hydroxycyclohexyl}morpholin-3-one trifluoroacetate
Example 36: (6S)-6-(4-{[(2R)-2-Benzylpiperazin-1-yl]carbonyl}-5-phenyl-1H-imidazol-1-yl)-1-oxa-3-azaspiro[4.5]decan-2-one
Example 37: 1-[(S)-(4-{[(2R)-2-Benzylpiperazin-1-yl]carbonyl}-5-phenyl-1H-imidazol-1-yl)(phenyl)methyl]cyclohexanol hydrochloride
Example 38: 1-[(R)-(4-{[(2R)-2-Benzylpiperazin-1-yl]carbonyl}-5-phenyl-1H-imidazol-1-yl)(phenyl)methyl]cyclohexanol
Example 39: (2R)-1-[(1-Cyclohexyl-5-phenyl-1H-imidazol-4-yl)carbonyl]-2-(2-phenoxyethyl)piperazine dihydrochloride
Example 40: 1-[(4-{[(2R)-2-(2-Phenoxyethyl)piperazin-1-yl]carbonyl}-5-phenyl-1H-imidazol-1-yl)methyl]cyclohexanol dihydrochloride
Example 41: 1-{[4-({(2R)-2-[2-(2-Methoxyphenoxy)ethyl]piperazin-1-yl}carbonyl)-5-phenyl-1H-imidazol-1-yl]methyl}cyclohexanol
Example 42: trans-4-(4-{[(2R)-2-Benzylpiperazin-1-yl]carbonyl}-5-phenyl-1H-imidazol-1-yl)cyclohexanol hydrochloride
Example 43: (1S,2S)-2-(4-{[(2R)-2-Benzylpiperazin-1-yl]carbonyl}-5-phenyl-1H-imidazol-1-yl)cyclohexanol
Example 44: (1R,2R)-2-(4-{[(2R)-2-Benzylpiperazin-1-yl]carbonyl}-5-phenyl-1H-imidazol-1-yl)cyclohexanol
Example 45: (2S)-2-(4-{[(2R)-2-Benzylpiperazin-1-yl]carbonyl}-5-phenyl-1H-imidazol-1-yl)cyclohexanone hydrochloride
Example 46: (2R)-2-(4-{[(2R)-2-Benzylpiperazin-1-yl]carbonyl}-5-phenyl-1H-imidazol-1-yl)cyclohexanone hydrochloride
Example 47: (1S,2S)-2-(4-{[(2R)-2-Benzylpiperazin-1-yl]carbonyl}-5-phenyl-1H-imidazol-1-yl)cyclohexyl acetate hydrochloride
Example 48: (1R,2S)-2-(4-{[(2R)-2-Benzylpiperazin-1-yl]carbonyl}-5-phenyl-1H-imidazol-1-yl)cyclohexyl 4-nitrobenzoate hydrochloride
Example 49: Ethyl [(1R,2S)-2-(4-{[(2R)-2-benzylpiperazin-1-yl]carbonyl}-5-phenyl-1H-imidazol-1-yl)cyclohexyl]carbamate
Example 50: (2R)-2-Benzyl-1-({1-[(1S,2R)-2-fluorocyclohexyl]-5-phenyl-1H-imidazol-4-yl}carbonyl)piperazine
Example 51: (2R)-1-({1-[(1S,2R)-2-Azidocyclohexyl]-5-phenyl-1H-imidazol-4-yl}carbonyl)-2-benzylpiperazine
Example 52: (1R,2S)-2-(4-{[(2R)-2-Benzylpiperazin-1-yl]carbonyl}-5-phenyl-1H-imidazol-1-yl)cyclohexanamine dihydrochloride
Example 53: (1R,2S)-2-(4-{[(2R)-2-Benzylpiperazin-1-yl]carbonyl}-5-phenyl-1H-imidazol-1-yl)-N-(cyclopropylmethyl)cyclohexanamine dihydrochloride
Example 54: (1R,2S)-2-(4-{[(2R)-2-Benzylpiperazin-1-yl]carbonyl}-5-phenyl-1H-imidazol-1-yl)-N,N-bis(cyclopropylmethyl)cyclohexanamine dihydrochloride
Example 55: N-[(1R,2S)-2-(4-{[(2R)-2-Benzylpiperazin-1-yl]carbonyl}-5-phenyl-1H-imidazol-1-yl)cyclohexyl]cyclopropanecarboxamide
Example 56: N-[(1R,2S)-2-(4-{[(2R)-2-Benzylpiperazin-1-yl]carbonyl}-5-phenyl-1H-imidazol-1-yl)cyclohexyl]cyclopropanesulfonamide
Example 57: N-[(1R,2S)-2-(4-{[(2R)-2-Benzylpiperazin-1-yl]carbonyl}-5-phenyl-1H-imidazol-1-yl)cyclohexyl]butanamide
Example 58: N-[(1R,2S)-2-(4-{[(2R)-2-Benzylpiperazin-1-yl]carbonyl}-5-phenyl-1H-imidazol-1-yl)cyclohexyl]-N′-ethylurea
Example 59: (1S,2S)-2-[4-{[(2R)-2-Benzylpiperazin-1-yl]carbonyl}-5-(2,3-difluorophenyl)-1H-imidazol-1-yl]cyclohexanol
Example 60: (1S,2S)-2-[4-{[(2R)-2-Benzylpiperazin-1-yl]carbonyl}-5-(3-fluorophenyl)-1H-imidazol-1-yl]cyclohexanol
Example 61: (1S,2S)-2-[4-{[(2R)-2-Benzylpiperazin-1-yl]carbonyl}-5-(4-fluorophenyl)-1H-imidazol-1-yl]cyclohexanol
Example 62: (2S)-2-(4-{[(2R)-2-Benzylpiperazin-1-yl]carbonyl}-5-phenyl-1H-imidazol-1-yl)-1-(trifluoromethyl)cyclohexanol hydrochloride
Example 63: (2R)-2-Benzyl-1-({1-[(1S,2S)-2-(3-methoxypropoxy)cycloheptyl]-5-phenyl-1H-imidazol-4-yl}carbonyl)piperazine hydrochloride
Example 64: (1S,2S)-2-(4-{[(2R)-2-Benzylpiperazin-1-yl]carbonyl}-5-phenyl-1H-imidazol-1-yl)cycloheptyl(2-furylmethyl)carbamate hydrochloride
Example 65: (2R)-2-Benzyl-1-({1-[(1S,2S)-2-(2-methoxyethoxy)cycloheptyl]-5-phenyl-1H-imidazol-4-yl}carbonyl)piperazine hydrochloride
Example 66: Ethyl [(2S)-2-(4-{[(2R)-2-benzylpiperazin-1-yl]carbonyl}-5-phenyl-1H-imidazol-1-yl)-1-hydroxycyclohexyl]acetate
Example 67: (2R)-2-Benzyl-1-({1-[(1S,2S)-2-(3-methoxypropoxy)cyclohexyl]-5-phenyl-1H-imidazol-4-yl}carbonyl)piperazine hydrochloride
Example 68: (2R)-1-({1-[(1S,2S)-2-(Allyloxy)cyclohexyl]-5-phenyl-1H-imidazol-4-yl}carbonyl)-2-benzylpiperazine hydrochloride
Example 69: (1S,2S)-2-(4-{[(2R)-2-Benzylpiperazin-1-yl]carbonyl}-5-phenyl-1H-imidazol-1-yl)cyclohexyl ethylcarbamate
Example 70: [(2S)-2-(4-{[(2R)-2-Benzylpiperazin-1-yl]carbonyl}-5-phenyl-1H-imidazol-1-yl)-1-hydroxycyclohexyl]acetic acid trifluoroacetate
Example 71: 2-[(2S)-2-(4-{[(2R)-2-Benzylpiperazin-1-yl]carbonyl}-5-phenyl-1H-imidazol-1-yl)-1-hydroxycyclohexyl]-N-methylacetamide
Example 72: 2-[(2S)-2-(4-{[(2R)-2-Benzylpiperazin-1-yl]carbonyl}-5-phenyl-1H-imidazol-1-yl)-1-hydroxycyclohexyl]-N,N-dimethylacetamide
Example 73: (1S,2S)-2-(4-{[(2R)-2-Benzylpiperazin-1-yl]carbonyl}-5-phenyl-1H-imidazol-1-yl)cyclohexyl (ethyl)(methyl)carbamate hydrochloride
Example 74: (1S,2S)-2-(4-{[(2R)-2-Benzylpiperazin-1-yl]carbonyl}-5-phenyl-1H-imidazol-1-yl)cyclohexyl methyl[2-(methylsulfonyl)ethyl]carbamate hydrochloride
Example 75: 2-(4-{[(2R)-2-Benzylpiperazin-1-yl]carbonyl}-5-phenyl-1H-imidazol-1-yl)-1-[(2-methoxyethoxy)methyl]cyclohexanol hydrochloride
Example 76: N-{2-[(2S)-2-(4-{[(2R)-2-Benzylpiperazin-1-yl]carbonyl}-5-phenyl-1H-imidazol-1-yl)-1-hydroxycyclohexyl]ethyl}acetamide trifluoroacetate
Example 77: 2-(4-{[(2R)-2-Benzylpiperazin-1-yl]carbonyl}-5-phenyl-1H-imidazol-1-yl)-1-[(ethylamino)methyl]cyclohexanol dihydrochloride
Example 78: 2-(4-{[(2R)-2-Benzylpiperazin-1-yl]carbonyl}-5-phenyl-1H-imidazol-1-yl)-1-[(3-methoxypropoxy)methyl]cyclohexanol hydrochloride
Example 79: (1S,2S)-2-(4-{[(2R)-2-Benzylpiperazin-1-yl]carbonyl}-5-phenyl-1H-imidazol-1-yl)cyclohexanamine
Example 80: N-(3-{[(1S,2S)-2-(4-{[(2R)-2-Benzylpiperazin-1-yl]carbonyl}-5-phenyl-1H-imidazol-1-yl)cyclohexyl]oxy}propyl)acetamide
Example 81: 2-(4-{[(2R)-2-Benzylpiperazin-1-yl]carbonyl}-5-phenyl-1H-imidazol-1-yl)-1-{[(ethyl)(methyl)amino]methyl}cyclohexanol dihydrochloride
Example 82: N-{[2-(4-{[(2R)-2-Benzylpiperazin-1-yl]carbonyl}-5-phenyl-1H-imidazol-1-yl)-1-hydroxycyclohexyl]methyl}-N-ethylacetamide
Example 83: (1S,2R)-2-(4-{[(2R)-2-Benzylpiperazin-1-yl]carbonyl}-5-phenyl-1H-imidazol-1-yl)-1-butylcyclohexanol hydrochloride
Example 84: (1R,2S)-2-(4-{[(2R)-2-Benzylpiperazin-1-yl]carbonyl}-5-phenyl-1H-imidazol-1-yl)-1-butylcyclohexanol hydrochloride
Example 85: 2-[(2S)-2-(4-{[(2R)-2-Benzylpiperazin-1-yl]carbonyl}-5-phenyl-1H-imidazol-1-yl)-1-hydroxycyclohexyl]-N-(2-furylmethyl)acetamide
Example 86: 2-(4-{[(2R)-2-Benzylpiperazin-1-yl]carbonyl}-5-phenyl-1H-imidazol-1-yl)-1-(propoxymethyl)cyclohexanol hydrochloride
Example 87: 2-(4-{[(2R)-2-Benzylpiperazin-1-yl]carbonyl}-5-phenyl-1H-imidazol-1-yl)-1-[(2,2-difluoroethoxy)methyl]cyclohexanol hydrochloride
Example 88: 2-(4-{[(2R)-2-Benzylpiperazin-1-yl]carbonyl}-5-phenyl-1H-imidazol-1-yl)-1-[(2,2,2-trifluoroethoxy)methyl]cyclohexanol hydrochloride
Example 89: (2R)-2-Benzyl-1-({5-phenyl-1-[(1S,2S)-2-propoxycyclohexyl]-1H-imidazol-4-yl}carbonyl)piperazine hydrochloride
Example 90: (2R)-2-Benzyl-1-({1-[(1S,2S)-2-(2-methoxyethoxy)cyclohexyl]-5-phenyl-1H-imidazol-4-yl}carbonyl)piperazine hydrochloride
Example 91: 2-(4-{[(2R)-2-Benzylpiperazin-1-yl]carbonyl}-5-phenyl-1H-imidazol-1-yl)-1-methylcyclohexanol hydrochloride
Example 92: (2R)-2-Benzyl-1-({1-[(1S,2S)-2-(4-methoxybutoxy)cyclohexyl]-5-phenyl-1H-imidazol-4-yl}carbonyl)piperazine hydrochloride
Example 93: 2-(4-{[(2R)-2-Benzylpiperazin-1-yl]carbonyl}-5-phenyl-1H-imidazol-1-yl)-1-[(ethylthio)methyl]cyclohexanol hydrochloride
Example 94: 2-(4-{[(2R)-2-Benzylpiperazin-1-yl]carbonyl}-5-phenyl-1H-imidazol-1-yl)-1-[(cyclopropylmethoxy)methyl]cyclohexanol hydrochloride
Example 95: 2-(4-{[(2R)-2-Benzylpiperazin-1-yl]carbonyl}-5-phenyl-1H-imidazol-1-yl)-1-[(cyclobutyloxy)methyl]cyclohexanol hydrochloride
Example 96: 1-(2-{[2-(4-{[(2R)-2-Benzylpiperazin-1-yl]carbonyl}-5-phenyl-1H-imidazol-1-yl)-1-hydroxycyclohexyl]methoxy}ethyl)pyrrolidin-2-one hydrochloride
Example 97: 3-(2-{[2-(4-{[(2R)-2-Benzylpiperazin-1-yl]carbonyl}-5-phenyl-1H-imidazol-1-yl)-1-hydroxycyclohexyl]methoxy}ethyl)-1,3-oxazolidin-2-one hydrochloride
Example 98: (2S)-2-(4-{[(2R)-2-Benzylpiperazin-1-yl]carbonyl}-5-phenyl-1H-imidazol-1-yl)-1-(2-hydroxyethyl)cyclohexanol hydrochloride
Example 99: (2R)-2-Benzyl-1-({1-[(1S)-2-methoxy-2-(2-methoxyethyl)cyclohexyl]-5-phenyl-1H-imidazol-4-yl}carbonyl)piperazine hydrochloride
Example 100: (2S)-2-(4-{[(2R)-2-Benzylpiperazin-1-yl]carbonyl}-5-phenyl-1H-imidazol-1-yl)-1-(2-methoxyethyl)cyclohexanol hydrochloride
Example 101: 2-(4-{[(2R)-2-Benzylpiperazin-1-yl]carbonyl}-5-phenyl-1H-imidazol-1-yl)-1-[(ethylsulfonyl)methyl]cyclohexanol hydrochloride
Example 102: (2E)-2-[(2S)-2-(4-{[(2R)-2-Benzylpiperazin-1-yl]carbonyl}-5-phenyl-1H-imidazol-1-yl)cyclohexylidene]-N-propylacetamide hydrochloride
Example 103: 2-(4-{[(2R)-2-Benzylpiperazin-1-yl]carbonyl}-5-phenyl-1H-imidazol-1-yl)-1-[(3-hydroxy-3-methylbutoxy)methyl]cyclohexanol hydrochloride
Example 104: 2-(4-{[(2R)-2-Benzylpiperazin-1-yl]carbonyl}-5-phenyl-1H-imidazol-1-yl)-1-(isopropoxymethyl)cyclohexanol hydrochloride
Example 105: 2-(4-{[(2R)-2-Benzylpiperazin-1-yl]carbonyl}-5-phenyl-1H-imidazol-1-yl)-1-[(tetrahydro-2H-pyran-4-yloxy)methyl]cyclohexanol hydrochloride
Example 106: 2-(4-{[(2R)-2-Benzylpiperazin-1-yl]carbonyl}-5-phenyl-1H-imidazol-1-yl)-1-ethylcyclohexanol hydrochloride
Example 107: 2-(4-{[(2R)-2-Benzylpiperazin-1-yl]carbonyl}-5-phenyl-1H-imidazol-1-yl)-1-propylcyclohexanol hydrochloride
Example 108: 3-[2-(4-{[(2R)-2-Benzylpiperazin-1-yl]carbonyl}-5-phenyl-1H-imidazol-1-yl)-1-hydroxycyclohexyl]propanenitrile
Example 109: 3-[(1S,2S)-2-(4-{[(2R)-2-Benzylpiperazin-1-yl]carbonyl}-5-phenyl-1H-imidazol-1-yl)cyclohexyl]-1,3-oxazolidin-2-one
Example 110: 2-(4-{[(2R)-2-Benzylpiperazin-1-yl]carbonyl}-5-phenyl-1H-imidazol-1-yl)-1-{[(3-methyloxetan-3-yl)methoxy]methyl}cyclohexanol
Example 111: 2-(4-{[(2R)-2-Benzylpiperazin-1-yl]carbonyl}-5-phenyl-1H-imidazol-1-yl)-1-[(1,3-thiazol-2-ylmethoxy)methyl]cyclohexanol hydrochloride
Example 112: 2-(4-{[(2R)-2-Benzylpiperazin-1-yl]carbonyl}-5-phenyl-1H-imidazol-1-yl)-1-{[(1-methyl-1H-imidazol-2-yl)methoxy]methyl}cyclohexanol dihydrochloride
Example 113: 2-(4-{[(2R)-2-Benzylpiperazin-1-yl]carbonyl}-5-phenyl-1H-imidazol-1-yl)-1-{[2-(methylthio)ethoxy]methyl}cyclohexanol hydrochloride
Example 114: 2-(4-{[(2R)-2-Benzylpiperazin-1-yl]carbonyl}-5-phenyl-1H-imidazol-1-yl)-1-{[3-(methylthio)propoxy]methyl}cyclohexanol hydrochloride
Example 115: 2-(4-{[(2R)-2-Benzylpiperazin-1-yl]carbonyl}-5-phenyl-1H-imidazol-1-yl)-1-[(tetrahydro-2H-thiopyran-4-yloxy)methyl]cyclohexanol hydrochloride
Example 116: 2-(4-{[(2R)-2-Benzylpiperazin-1-yl]carbonyl}-5-phenyl-1H-imidazol-1-yl)-1-[(tetrahydro-2H-thiopyran-4-ylmethoxy)methyl]cyclohexanol hydrochloride
Example 117: 2-(4-{[(2R)-2-Benzylpiperazin-1-yl]carbonyl}-5-phenyl-1H-imidazol-1-yl)-1-[(tetrahydro-2H-pyran-4-ylmethoxy)methyl]cyclohexanol hydrochloride
Example 118: 2-(4-{[(2R)-2-Benzylpiperazin-1-yl]carbonyl}-5-phenyl-1H-imidazol-1-yl)-1-{[2-(methylsulfonyl)ethoxy]methyl}cyclohexanol hydrochloride
Example 119: 2-(4-{[(2R)-2-Benzylpiperazin-1-yl]carbonyl}-5-phenyl-1H-imidazol-1-yl)-1-{[3-(methylsulfonyl)propoxy]methyl}cyclohexanol hydrochloride
Example 120: 2-(4-{[(2R)-2-Benzylpiperazin-1-yl]carbonyl}-5-phenyl-1H-imidazol-1-yl)-1-{[(1,1-dioxidotetrahydro-2H-thiopyran-4-yl)methoxy]methyl}cyclohexanol hydrochloride
Example 121: 2-(4-{[(2R)-2-Benzylpiperazin-1-yl]carbonyl}-5-phenyl-1H-imidazol-1-yl)-1-(phenoxymethyl)cyclohexanol hydrochloride
Example 122: 2-(4-{[(2R)-2-Benzylpiperazin-1-yl]carbonyl}-5-phenyl-1H-imidazol-1-yl)-1-{[(2-furylmethyl)amino]methyl}cyclohexanol hydrochloride
Example 123: (1R,2S)-2-(4-{[(2R)-2-Benzylpiperazin-1-yl]carbonyl}-5-phenyl-1H-imidazol-1-yl)-1-[(1,3-thiazol-2-ylmethoxy)methyl]cyclohexanol hydrochloride
Example 124: Ethyl [(1R,2R)-2-(4-{[(2R)-2-benzylpiperazin-1-yl]carbonyl}-5-phenyl-1H-imidazol-1-yl)cyclohexyl]carbamate
Example 125: (1R,2S)-2-(4-{[(2R)-2-Benzylpiperazin-1-yl]carbonyl}-5-phenyl-1H-imidazol-1-yl)-1-{[2-(2-hydroxyethoxy)ethoxy]methyl}cyclohexanol
Example 126: (1R,2S)-2-(4-{[(2R)-2-Benzylpiperazin-1-yl]carbonyl}-5-phenyl-1H-imidazol-1-yl)-1-{[3-(dimethylamino)propoxy]methyl}cyclohexanol hydrochloride
Example 127: (1R,2S)-2-(4-{[(2R)-2-Benzylpiperazin-1-yl]carbonyl}-5-phenyl-1H-imidazol-1-yl)-1-[(pyridin-2-ylmethoxy)methyl]cyclohexanol dihydrochloride
Example 128: (1R,2S)-1-[(1H-Benzimidazol-2-ylmethoxy)methyl]-2-(4-{[(2R)-2-benzylpiperazin-1-yl]carbonyl}-5-phenyl-1H-imidazol-1-yl)cyclohexanol
Example 129: (1R,2S)-2-(4-{[(2R)-2-Benzylpiperazin-1-yl]carbonyl}-5-phenyl-1H-imidazol-1-yl)-1-[(2,3-dihydro-1H-inden-2-yloxy)methyl]cyclohexanol hydrochloride
Example 130: Ethyl [(1S,2S)-2-(4-{[(2R)-2-benzylpiperazin-1-yl]carbonyl}-5-phenyl-1H-imidazol-1-yl)cyclohexyl](methyl)carbamate
Example 131: Ethyl [(1S,2S)-2-(4-{[(2R)-2-benzylpiperazin-1-yl]carbonyl}-5-phenyl-1H-imidazol-1-yl)cyclohexyl](3-methoxypropyl)carbamate
Example 132: Ethyl {[2-(4-{[(2R)-2-benzylpiperazin-1-yl]carbonyl}-5-phenyl-1H-imidazol-1-yl)-1-ethoxycyclohexyl]methyl}carbamate
Example 133: (1R,2S)-2-(4-{[(2R)-2-(4-Fluorobenzyl)piperazin-1-yl]carbonyl}-5-phenyl-1H-imidazol-1-yl)cyclohexanol hydrochloride
Example 134: (1R,2S)-2-(5-Phenyl-4-{[(2S)-2-(2,2,2-trifluoro-1-hydroxyethyl)piperazin-1-yl]carbonyl}-1H-imidazol-1-yl)cyclohexanol
Example 135: (1R,2S)-2-[4-{[(2R)-2-Benzylpiperazin-1-yl]carbonyl}-5-(2-fluorophenyl)-1H-imidazol-1-yl]cyclohexanol
Example 136: (1R,2S)-2-[4-{[(2R)-2-Benzylpiperazin-1-yl]carbonyl}-5-(3,5-difluorophenyl)-1H-imidazol-1-yl]cyclohexanol
Example 137: (1R,2S)-2-(4-{[(2R)-2-(2-Fluorobenzyl)piperazin-1-yl]carbonyl}-5-phenyl-1H-imidazol-1-yl)cyclohexanol hydrochloride
Example 138: (1R,2S)-2-(4-{[(2R)-2-(3-Fluorobenzyl)piperazin-1-yl]carbonyl}-5-phenyl-1H-imidazol-1-yl)cyclohexanol hydrochloride
Example 139: (1R,2S)-2-(4-{[(2R)-2-(3,4-Difluorobenzyl)piperazin-1-yl]carbonyl}-5-phenyl-1H-imidazol-1-yl)cyclohexanol hydrochloride
Example 140: (1R,2S)-2-[5-Phenyl-4-({(2R)-2-[3-(trifluoromethyl)benzyl]piperazin-1-yl}carbonyl)-1H-imidazol-1-yl]cyclohexanol hydrochloride
Example 141: (1R,2S)-2-[5-Phenyl-4-({(2R)-2-[4-(trifluoromethyl)benzyl]piperazin-1-yl}carbonyl)-1H-imidazol-1-yl]cyclohexanol hydrochloride
Example 142: (1R,2S)-2-(4-{[(2R)-2-(2,3-Dihydro-1H-inden-2-yl)piperazin-1-yl]carbonyl}-5-phenyl-1H-imidazol-1-yl)cyclohexanol hydrochloride
Example 143: 2-{[(2S)-1-({1-[(1S,2R)-2-Hydroxycyclohexyl]-5-phenyl-1H-imidazol-4-yl}carbonyl)piperazin-2-yl]methoxy}benzonitrile
Example 144: (1R,2S)-2-(4-{[(2S)-2-(Phenoxymethyl)piperazin-1-yl]carbonyl}-5-phenyl-1H-imidazol-1-yl)cyclohexanol
Example 145: (1R,2S)-2-(4-{[(2S)-2-(Phenoxymethyl)piperazin-1-yl]carbonyl}-5-phenyl-1H-imidazol-1-yl)cyclohexanol hydrochloride
Example 146: (1R,2S)-2-(5-Phenyl-4-{[(2R)-2-(2,4,5-trifluorobenzyl)piperazin-1-yl]carbonyl}-1H-imidazol-1-yl)cyclohexanol hydrochloride
Example 147: (1R,2S)-2-[5-Phenyl-4-({(2R)-2-[2-(trifluoromethyl)benzyl]piperazin-1-yl}carbonyl)-1H-imidazol-1-yl]cyclohexanol hydrochloride
Example 148: (1R,2S)-2-[4-({(2S)-2-[(3,5-Difluorophenoxy)methyl]piperazin-1-yl}carbonyl)-5-phenyl-1H-imidazol-1-yl]cyclohexanol hydrochloride
Example 149: (1R,2S)-2-[4-({(2S)-2-[(2,6-Difluorophenoxy)methyl]piperazin-1-yl}carbonyl)-5-phenyl-1H-imidazol-1-yl]cyclohexanol hydrochloride
Example 150: (1R,2S)-2-[5-Phenyl-4-({(2S)-2-[(pyridin-2-yloxy)methyl]piperazin-1-yl}carbonyl)-1H-imidazol-1-yl]cyclohexanol dihydrochloride
Example 151: (1R,2S)-2-(4-{[(2R)-2-(2-Phenoxyethyl)piperazin-1-yl]carbonyl}-5-phenyl-1H-imidazol-1-yl)cyclohexanol
Example 152: Isopropyl [(1S,2S)-2-(4-{[(2R)-2-benzylpiperazin-1-yl]carbonyl}-5-phenyl-1H-imidazol-1-yl)cyclohexyl]carbamate
Example 153: Isobutyl [(1S,2S)-2-(4-{[(2R)-2-benzylpiperazin-1-yl]carbonyl}-5-phenyl-1H-imidazol-1-yl)cyclohexyl]carbamate
Example 154: 2-Methoxyethyl [(1S,2S)-2-(4-{[(2R)-2-benzylpiperazin-1-yl]carbonyl}-5-phenyl-1H-imidazol-1-yl)cyclohexyl]carbamate
Example 155: Ethyl [(1S,2S)-2-(4-{[(2R)-2-(2-fluorobenzyl)piperazin-1-yl]carbonyl}-5-phenyl-1H-imidazol-1-yl)cyclohexyl]carbamate
Example 156: Ethyl [(1S,2S)-2-(4-{[(2R)-2-(3-fluorobenzyl)piperazin-1-yl]carbonyl}-5-phenyl-1H-imidazol-1-yl)cyclohexyl]carbamate
Example 157: Ethyl [(1S,2S)-2-(4-{[(2R)-2-(4-fluorobenzyl)piperazin-1-yl]carbonyl}-5-phenyl-1H-imidazol-1-yl)cyclohexyl]carbamate
Example 158: Ethyl [(1S,2S)-2-(4-{[(2R)-2-(4-cyanobenzyl)piperazin-1-yl]carbonyl}-5-phenyl-1H-imidazol-1-yl)cyclohexyl]carbamate
Example 159: Methyl [(1S,2S)-2-(4-{[(2R)-2-(3,5-difluorobenzyl)piperazin-1-yl]carbonyl}-5-phenyl-1H-imidazol-1-yl)cyclohexyl]carbamate
Example 160: Methyl [(1S,2S)-2-(4-{[(2R)-2-(4-cyanobenzyl)piperazin-1-yl]carbonyl}-5-phenyl-1H-imidazol-1-yl)cyclohexyl]carbamate
Example 161: Methyl {(1S,2S)-2-[4-{[(2R)-2-benzylpiperazin-1-yl]carbonyl}-5-(3-fluorophenyl)-1H-imidazol-1-yl]cyclohexyl}carbamate
Example 162: Ethyl {(1S,2S)-2-[4-{[(2R)-2-benzylpiperazin-1-yl]carbonyl}-5-(3-fluorophenyl)-1H-imidazol-1-yl]cyclohexyl}carbamate
Example 163: Methyl [(1S,2S)-2-(5-phenyl-4-{[(2R)-2-(2-phenylethyl)piperazin-1-yl]carbonyl}-1H-imidazol-1-yl)cyclohexyl]carbamate
Example 164: Methyl {(1S,2S)-2-[5-phenyl-4-({(2S)-2-[(phenylthio)methyl]piperazin-1-yl}carbonyl)-1H-imidazol-1-yl]cyclohexyl}carbamate
Example 165: Methyl [(1S,2S)-2-(4-{[(2S)-2-(phenoxymethyl)piperazin-1-yl]carbonyl}-5-phenyl-1H-imidazol-1-yl)cyclohexyl]carbamate
Example 166: Methyl [(1S,2S)-2-(4-{[(2R)-2-(2-phenoxyethyl)piperazin-1-yl]carbonyl}-5-phenyl-1H-imidazol-1-yl)cyclohexyl]carbamate
Example 167: Methyl {(1S,2S)-2-[4-({(2R)-2-[2-(1H-indazol-1-yl)ethyl]piperazin-1-yl}carbonyl)-5-phenyl-1H-imidazol-1-yl]cyclohexyl}carbamate
Example 168: Ethyl [(1S,2S)-2-(4-{[(2R)-2-(2-phenoxyethyl)piperazin-1-yl]carbonyl}-5-phenyl-1H-imidazol-1-yl)cyclohexyl]carbamate
Example 169: 4-{[(2R)-1-({1-[(1R,2S)-2-Hydroxy-2-(methoxymethyl)cyclohexyl]-5-phenyl-1H-imidazol-4-yl}carbonyl)piperazin-2-yl]methyl}benzonitrile hydrochloride
Example 170: (1S,2R)-2-(4-{[(2R)-2-(Cyclohexylmethyl)piperazin-1-yl]carbonyl}-5-phenyl-1H-imidazol-1-yl)-1-(methoxymethyl)cyclohexanol hydrochloride
Example 171: (1S,2R)-2-(4-{[(2R)-2-Isobutylpiperazin-1-yl]carbonyl}-5-phenyl-1H-imidazol-1-yl)-1-(methoxymethyl)cyclohexanol hydrochloride
Example 172: (1S,2R)-2-(4-{[(2R)-2-Isopropylpiperazin-1-yl]carbonyl}-5-phenyl-1H-imidazol-1-yl)-1-(methoxymethyl)cyclohexanol hydrochloride
Example 173: (1S,2R)-2-[4-({(2S)-2-[(Cyclopropyl)(hydroxy)methyl]piperazin-1-yl}carbonyl)-5-phenyl-1H-imidazol-1-yl]-1-(methoxymethyl)cyclohexanol hydrochloride
Example 174: (1S,2R)-1-(Methoxymethyl)-2-(5-phenyl-4-{[(2R)-2-(pyridin-2-ylmethyl)piperazin-1-yl]carbonyl}-1H-imidazol-1-yl)cyclohexanol
Example 175: (1S,2R)-1-(Methoxymethyl)-2-(5-phenyl-4-{[(2R)-2-(pyridin-4-ylmethyl)piperazin-1-yl]carbonyl}-1H-imidazol-1-yl)cyclohexanol dihydrochloride
Example 176: (1S,2R)-2-[4-{[(2R)-2-(3,5-Difluorobenzyl)piperazin-1-yl]carbonyl}-5-(3-fluorophenyl)-1H-imidazol-1-yl]-1-(methoxymethyl)cyclohexanol
Example 177: (1S,2R)-2-[4-{[(2R)-2-(4-Fluorobenzyl)piperazin-1-yl]carbonyl}-5-(3-fluorophenyl)-1H-imidazol-1-yl]-1-(methoxymethyl)cyclohexanol
Example 178: (1S,2R)-2-[4-{[(2R)-2-(3-Fluorobenzyl)piperazin-1-yl]carbonyl}-5-(3-fluorophenyl)-1H-imidazol-1-yl]-1-(methoxymethyl)cyclohexanol
Example 179: (1S,2R)-2-(4-{[(2R)-2-(4-Methoxybenzyl)piperazin-1-yl]carbonyl}-5-phenyl-1H-imidazol-1-yl)-1-(methoxymethyl)cyclohexanol hydrochloride
Example 180: (1S,2R)-2-(4-{[(2R)-2-(1H-Indol-3-ylmethyl)piperazin-1-yl]carbonyl}-5-phenyl-1H-imidazol-1-yl)-1-(methoxymethyl)cyclohexanol hydrochloride
Example 181: (1S,2R)-2-[4-{[(2R)-2-(2-Fluorobenzyl)piperazin-1-yl]carbonyl}-5-(3-fluorophenyl)-1H-imidazol-1-yl]-1-(methoxymethyl)cyclohexanol
Example 182: 4-{[(2R)-1-({5-(3-Fluorophenyl)-1-[(1R,2S)-2-hydroxy-2-(methoxymethyl)cyclohexyl]-1H-imidazol-4-yl}carbonyl)piperazin-2-yl]methyl}benzonitrile
Example 183: (1S,2R)-1-(Methoxymethyl)-2-(5-phenyl-4-{[(2R)-2-(1,3-thiazol-4-ylmethyl)piperazin-1-yl]carbonyl}-1H-imidazol-1-yl)cyclohexanol dihydrochloride
Example 184: (1S,2R)-1-(Methoxymethyl)-2-(5-phenyl-4-{[(2R)-2-(2-phenylethyl)piperazin-1-yl]carbonyl}-1H-imidazol-1-yl)cyclohexanol hydrochloride
Example 185: (1S,2R)-1-(Methoxymethyl)-2-(4-{[(2R)-2-(4-morpholinobenzyl)piperazin-1-yl]carbonyl}-5-phenyl-1H-imidazol-1-yl)cyclohexanol dihydrochloride
Example 186: (1S,2R)-2-(4-{[(2R)-2-(2,2-Dimethylpropyl)piperazin-1-yl]carbonyl}-5-phenyl-1H-imidazol-1-yl)-1-(methoxymethyl)cyclohexanol hydrochloride
Example 187: (1S,2R)-1-(Methoxymethyl)-2-[4-({(2S)-2-[(4-methyl-1H-pyrazol-1-yl)methyl]piperazin-1-yl}carbonyl)-5-phenyl-1H-imidazol-1-yl]cyclohexanol
Example 188: (1S,2R)-1-(Methoxymethyl)-2-(5-phenyl-4-{[(2S)-2-(1H-1,2,4-triazol-1-ylmethyl)piperazin-1-yl]carbonyl}-1H-imidazol-1-yl)cyclohexanol
Example 189: (1S,2R)-1-(Methoxymethyl)-2-(4-{[(2S)-2-(phenoxymethyl)piperazin-1-yl]carbonyl}-5-phenyl-1H-imidazol-1-yl)cyclohexanol hydrochloride
Example 190: (1S,2R)-2-(5-(3-Fluorophenyl)-4-{[(2R)-2-(pyridin-3-ylmethyl)piperazin-1-yl]carbonyl}-1H-imidazol-1-yl)-1-(methoxymethyl)cyclohexanol
Example 191: (1S,2R)-2-(4-{[(2R)-2-(3-Methoxybenzyl)piperazin-1-yl]carbonyl}-5-phenyl-1H-imidazol-1-yl)-1-(methoxymethyl)cyclohexanol hydrochloride
Example 192: 3,5-Difluoro-N-{[(2S)-1-({1-[(1R,2S)-2-hydroxy-2-(methoxymethyl)cyclohexyl]-5-phenyl-1H-imidazol-4-yl}carbonyl)piperazin-2-yl]methyl}benzamide hydrochloride
Example 193: (1S,2R)-1-(Methoxymethyl)-2-(5-phenyl-4-{[(2S)-2-(1H-pyrazol-1-ylmethyl)piperazin-1-yl]carbonyl}-1H-imidazol-1-yl)cyclohexanol hydrochloride
Example 194: (1S,2R)-2-(4-{[(2S)-2-(1H-Indazol-1-ylmethyl)piperazin-1-yl]carbonyl}-5-phenyl-1H-imidazol-1-yl)-1-(methoxymethyl)cyclohexanol hydrochloride
Example 195: (1S,2R)-2-(4-{[(2S)-2-(1H-1,2,3-Benzotriazol-1-ylmethyl)piperazin-1-yl]carbonyl}-5-phenyl-1H-imidazol-1-yl)-1-(methoxymethyl)cyclohexanol
Example 196: (1S,2R)-1-(Methoxymethyl)-2-(5-phenyl-4-{[(2S)-2-({[6-(trifluoromethyl)pyridin-2-yl]oxy}methyl)piperazin-1-yl]carbonyl}-1H-imidazol-1-yl)cyclohexanol
Example 197: (1S,2R)-1-(Methoxymethyl)-2-(5-phenyl-4-{[(2S)-2-({[4-(trifluoromethyl)pyridin-2-yl]oxy}methyl)piperazin-1-yl]carbonyl}-1H-imidazol-1-yl)cyclohexanol
Example 198: Methyl 6-{[(2S)-1-({1-[(1R,2S)-2-hydroxy-2-(methoxymethyl)cyclohexyl]-5-phenyl-1H-imidazol-4-yl}carbonyl)piperazin-2-yl]methoxy}nicotinate trihydrochloride
Example 199: (1S,2R)-2-{4-[((2R)-2-{(2R)-2-Hydroxy-2-[6-(trifluoromethyl)pyridin-2-yl]ethyl}piperazin-1-yl)carbonyl]-5-phenyl-1H-imidazol-1-yl}-1-(methoxymethyl)cyclohexanol trihydrochloride
Example 200: (1S,2R)-2-{4-[((2R)-2-{(2S)-2-Hydroxy-2-[6-(trifluoromethyl)pyridin-2-yl]ethyl}piperazin-1-yl)carbonyl]-5-phenyl-1H-imidazol-1-yl}-1-(methoxymethyl)cyclohexanol trihydrochloride
Example 201: (1S,2R)-2-(4-{[(2S)-2-(1H-Imidazol-1-ylmethyl)piperazin-1-yl]carbonyl}-5-phenyl-1H-imidazol-1-yl)-1-(methoxymethyl)cyclohexanol trihydrochloride
Example 202: (1S,2R)-2-[4-({(2S)-2-[(3,5-Dimethyl-1H-pyrazol-1-yl)methyl]piperazin-1-yl}carbonyl)-5-phenyl-1H-imidazol-1-yl]-1-(methoxymethyl)cyclohexanol dihydrochloride
Example 203: (1S,2R)-1-(Methoxymethyl)-2-{5-phenyl-4-[((2S)-2-{[3-(trifluoromethyl)-1H-pyrazol-1-yl]methyl}piperazin-1-yl)carbonyl]-1H-imidazol-1-yl}cyclohexanol dihydrochloride
Example 204: (1S,2R)-2-(4-{[(2S)-2-(1H-Benzimidazol-1-ylmethyl)piperazin-1-yl]carbonyl}-5-phenyl-1H-imidazol-1-yl)-1-(methoxymethyl)cyclohexanol
Example 205: (1S,2R)-2-[4-({(2R)-2-[(2R)-2-Hydroxy-2-phenylethyl]piperazin-1-yl}carbonyl)-5-phenyl-1H-imidazol-1-yl]-1-(methoxymethyl)cyclohexanol dihydrochloride
Example 206: (1S,2R)-2-[4-({(2R)-2-[(2S)-2-Hydroxy-2-phenylethyl]piperazin-1-yl}carbonyl)-5-phenyl-1H-imidazol-1-yl]-1-(methoxymethyl)cyclohexanol dihydrochloride
Example 207: (1S,2R)-1-(Methoxymethyl)-2-[5-phenyl-4-({(2R)-2-[(5-phenyl-1,3,4-oxadiazol-2-yl)methyl]piperazin-1-yl}carbonyl)-1H-imidazol-1-yl]cyclohexanol hydrochloride
Example 208: (1S,2R)-2-(4-{[(2R)-2-(1H-Benzimidazol-2-ylmethyl)piperazin-1-yl]carbonyl}-5-phenyl-1H-imidazol-1-yl)-1-(methoxymethyl)cyclohexanol
Example 209: (1S,2R)-1-(Methoxymethyl)-2-{5-phenyl-4-[((2R)-2-{2-[4-(trifluoromethyl)phenyl]ethyl}piperazin-1-yl)carbonyl]-1H-imidazol-1-yl}cyclohexanol
Example 210: (1S,2R)-2-{5-(3-Fluorophenyl)-4-[((2R)-2-{2-[4-(methylsulfonyl)phenoxy]ethyl}piperazin-1-yl)carbonyl]-1H-imidazol-1-yl}-1-(methoxymethyl)cyclohexanol
Example 211: (1S,2R)-2-(4-{[(2R)-2-(3-Hydroxypropyl)piperazin-1-yl]carbonyl}-5-phenyl-1H-imidazol-1-yl)-1-(methoxymethyl)cyclohexanol
Example 212: (1S,2R)-1-(Methoxymethyl)-2-(4-{[(2R)-2-(3-phenoxypropyl)piperazin-1-yl]carbonyl}-5-phenyl-1H-imidazol-1-yl)cyclohexanol
Example 213: (1S,2R)-2-[4-({(2R)-2-[3-(1H-Indazol-1-yl)propyl]piperazin-1-yl}carbonyl)-5-phenyl-1H-imidazol-1-yl]-1-(methoxymethyl)cyclohexanol
Example 214: (1S,2R)-2-[4-({(2R)-2-[3-(2H-Indazol-2-yl)propyl]piperazin-1-yl}carbonyl)-5-phenyl-1H-imidazol-1-yl]-1-(methoxymethyl)cyclohexanol
Example 215: Ethyl 1-{3-[(2R)-1-({1-[(1R,2S)-2-hydroxy-2-(methoxymethyl)cyclohexyl]-5-phenyl-1H-imidazol-4-yl}carbonyl)piperazin-2-yl]propyl}-3-methyl-1H-pyrazole-5-carboxylate
Example 216: (1S,2R)-2-(4-{[(2S)-2-Benzylpiperazin-1-yl]carbonyl}-5-phenyl-1H-imidazol-1-yl)-1-(methoxymethyl)cyclohexanol
Example 217: (1S,2R)-2-[5-(3-Fluorophenyl)-4-({(2R)-2-[2-(2-methoxyphenoxy)ethyl]piperazin-1-yl}carbonyl)-1H-imidazol-1-yl]-1-(methoxymethyl)cyclohexanol
Example 218: (1S,2R)-2-(4-{[(2R)-2-(2-Hydroxyethyl)piperazin-1-yl]carbonyl}-5-phenyl-1H-imidazol-1-yl)-1-(methoxymethyl)cyclohexanol
Example 219: (1S,2R)-2-[4-({(2R)-2-[2-(Cyclopropylmethoxy)ethyl]piperazin-1-yl}carbonyl)-5-phenyl-1H-imidazol-1-yl]-1-(methoxymethyl)cyclohexanol
Example 220: (1S,2R)-1-(Methoxymethyl)-2-(4-{[(2R)-2-(2-{[1-methyl-5-(trifluoromethyl)-1H-pyrazol-3-yl]oxy}ethyl)piperazin-1-yl]carbonyl}-5-phenyl-1H-imidazol-1-yl)cyclohexanol dihydrochloride
Example 221: (1S,2R)-1-(Methoxymethyl)-2-{5-phenyl-4-[((2R)-2-{2-[2-(trifluoromethoxy)phenoxy]ethyl}piperazin-1-yl)carbonyl]-1H-imidazol-1-yl}cyclohexanol dihydrochloride
Example 222: (1S,2R)-1-(Methoxymethyl)-2-(4-{[(2R)-2-(2-{[1-methyl-3-(trifluoromethyl)-1H-pyrazol-5-yl]oxy}ethyl)piperazin-1-yl]carbonyl}-5-phenyl-1H-imidazol-1-yl)cyclohexanol dihydrochloride
Example 223: (1S,2R)-1-(Methoxymethyl)-2-[5-phenyl-4-({(2R)-2-[2-(pyridin-2-yloxy)ethyl]piperazin-1-yl}carbonyl)-1H-imidazol-1-yl]cyclohexanol trihydrochloride
Example 224: (1S,2R)-1-(Methoxymethyl)-2-(5-phenyl-4-{[(2R)-2-(2-{[6-(trifluoromethyl)pyridin-2-yl]oxy}ethyl)piperazin-1-yl]carbonyl}-1H-imidazol-1-yl)cyclohexanol trihydrochloride
Example 225: (1S,2R)-1-(Methoxymethyl)-2-[5-phenyl-4-({(2R)-2-[2-(pyrimidin-2-yloxy)ethyl]piperazin-1-yl}carbonyl)-1H-imidazol-1-yl]cyclohexanol dihydrochloride
Example 226: (1S,2R)-1-(Methoxymethyl)-2-(5-phenyl-4-{[(2R)-2-(2-{[4-(trifluoromethyl)pyridin-2-yl]oxy}ethyl)piperazin-1-yl]carbonyl}-1H-imidazol-1-yl)cyclohexanol trihydrochloride
Example 227: (1S,2R)-1-(Methoxymethyl)-2-(5-phenyl-4-{[(2R)-2-(2-{[3-(trifluoromethyl)pyridin-2-yl]oxy}ethyl)piperazin-1-yl]carbonyl}-1H-imidazol-1-yl)cyclohexanol trihydrochloride
Example 228: (1S,2R)-1-(Methoxymethyl)-2-[4-({(2R)-2-[2-(4-methoxyphenoxy)ethyl]piperazin-1-yl}carbonyl)-5-phenyl-1H-imidazol-1-yl]cyclohexanol
Example 229: Methyl 3-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethoxy}benzoate
Example 230: 6-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}nicotinonitrile trihydrochloride
Example 231: 1-(4-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}phenyl)ethanone
Example 232: 1-(4-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}phenyl)ethanone dihydrochloride
Example 233: 1-(3-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}phenyl)ethanone
Example 234: (1S,2R)-2-{4-[((2R)-2-{2-[4-(1H-Imidazol-1-yl)phenoxy]ethyl}piperazin-1-yl)carbonyl]-5-phenyl-1H-imidazol-1-yl}-1-(methoxymethyl)cyclohexanol
Example 235: (1S,2R)-2-[4-({(2R)-2-[2-(1,2-Benzisoxazol-3-yloxy)ethyl]piperazin-1-yl}carbonyl)-5-phenyl-1H-imidazol-1-yl]-1-(methoxymethyl)cyclohexanol dihydrochloride
Example 236: (1S,2R)-1-(Methoxymethyl)-2-{4-[((2R)-2-{2-[3-(2-methyl-1H-imidazol-1-yl)phenoxy]ethyl}piperazin-1-yl)carbonyl]-5-phenyl-1H-imidazol-1-yl}cyclohexanol
Example 237: Methyl 3-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethoxy}isoxazole-5-carboxylate bistrifluoroacetate
Example 238: (1S,2R)-1-(Methoxymethyl)-2-{5-phenyl-4-[((2R)-2-{2-[4-(1H-pyrazol-1-yl)phenoxy]ethyl}piperazin-1-yl)carbonyl]-1H-imidazol-1-yl}cyclohexanol
Example 239: 2-[(2R)-1-({1-[(1R,2S)-2-Hydroxy-2-(methoxymethyl)cyclohexyl]-5-phenyl-1H-imidazol-4-yl}carbonyl)piperazin-2-yl]ethyl pyrrolidine-1-carboxylate dihydrochloride
Example 240: 1-(2-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}phenyl)ethanone
Example 241: Methyl 2-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethoxy}benzoate
Example 242: 4-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}-N,N-dimethylbenzamide
Example 243: (1S,2R)-2-{4-[((2R)-2-{2-[4-(Azetidin-1-ylcarbonyl)phenoxy]ethyl}piperazin-1-yl)carbonyl]-5-phenyl-1H-imidazol-1-yl}-1-(methoxymethyl)cyclohexanol
Example 244: (1S,2R)-2-[4-({(2R)-2-[2-(3-Fluorophenoxy)ethyl]piperazin-1-yl}carbonyl)-5-phenyl-1H-imidazol-1-yl]-1-(methoxymethyl)cyclohexanol
Example 245: (1S,2R)-2-[4-({(2R)-2-[2-(4-Fluorophenoxy)ethyl]piperazin-1-yl}carbonyl)-5-phenyl-1H-imidazol-1-yl]-1-(methoxymethyl)cyclohexanol
Example 246: (1S,2R)-1-(Methoxymethyl)-2-[4-({(2R)-2-[2-(2-methoxyphenoxy)ethyl]piperazin-1-yl}carbonyl)-5-phenyl-1H-imidazol-1-yl]cyclohexanol
Example 247: (1S,2R)-1-(Methoxymethyl)-2-[4-({(2R)-2-[2-(3-methoxyphenoxy)ethyl]piperazin-1-yl}carbonyl)-5-phenyl-1H-imidazol-1-yl]cyclohexanol
Example 248: (1S,2R)-1-(Methoxymethyl)-2-(5-phenyl-4-{[(2R)-2-(2-{4-[(trifluoromethyl)sulfonyl]phenoxy}ethyl)piperazin-1-yl]carbonyl}-1H-imidazol-1-yl)cyclohexanol
Example 249: (1S,2R)-1-(Methoxymethyl)-2-{4-[((2R)-2-{2-[4-(methylsulfonyl)phenoxy]ethyl}piperazin-1-yl)carbonyl]-5-phenyl-1H-imidazol-1-yl}cyclohexanol
Example 250: (1S,2R)-2-{4-[((2R)-2-{2-[(2,2-Dimethyl-2,3-dihydro-1-benzofuran-7-yl)oxy]ethyl}piperazin-1-yl)carbonyl]-5-phenyl-1H-imidazol-1-yl}-1-(methoxymethyl)cyclohexanol hydrochloride
Example 251: 6-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}-3,4-dihydroquinolin-2(1H)-one
Example 252: (1S,2R)-1-(Methoxymethyl)-2-(5-phenyl-4-{[(2R)-2-(2-{[5-(trifluoromethyl)pyridin-2-yl]oxy}ethyl)piperazin-1-yl]carbonyl}-1H-imidazol-1-yl)cyclohexanol trihydrochloride
Example 253: Methyl 5-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethoxy}nicotinate dihydrochloride
Example 254: 6-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}-3,4-dihydronaphthalen-1(2H)-one hydrochloride
Example 255: (1S,2R)-2-[4-({(2R)-2-[2-(3-Chlorophenoxy)ethyl]piperazin-1-yl}carbonyl)-5-phenyl-1H-imidazol-1-yl]-1-(methoxymethyl)cyclohexanol
Example 256: (1S,2R)-2-[4-({(2R)-2-[2-(4-Chlorophenoxy)ethyl]piperazin-1-yl}carbonyl)-5-phenyl-1H-imidazol-1-yl]-1-(methoxymethyl)cyclohexanol
Example 257: (1S,2R)-2-[4-({(2R)-2-[2-(4-Bromo-2-fluorophenoxy)ethyl]piperazin-1-yl}carbonyl)-5-phenyl-1H-imidazol-1-yl]-1-(methoxymethyl)cyclohexanol
Example 258: (1S,2R)-1-(Methoxymethyl)-2-{5-phenyl-4-[((2R)-2-{2-[4-(1H-1,2,3-triazol-1-yl)phenoxy]ethyl}piperazin-1-yl)carbonyl]-1H-imidazol-1-yl}cyclohexanol dihydrochloride
Example 259: (1S,2R)-1-(Methoxymethyl)-2-{4-[((2R)-2-{2-[4-(5-methyl-1,3,4-oxadiazol-2-yl)phenoxy]ethyl}piperazin-1-yl)carbonyl]-5-phenyl-1H-imidazol-1-yl}cyclohexanol dihydrochloride
Example 260: (1S,2R)-1-(Methoxymethyl)-2-[4-({(2R)-2-[2-(4-methylphenoxy)ethyl]piperazin-1-yl}carbonyl)-5-phenyl-1H-imidazol-1-yl]cyclohexanol
Example 261: Methyl (4-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethoxy}phenyl)acetate
Example 262: (1S,2R)-2-{4-[((2R)-2-{2-[3-(Diethylamino)phenoxy]ethyl}piperazin-1-yl)carbonyl]-5-phenyl-1H-imidazol-1-yl}-1-(methoxymethyl)cyclohexanol dihydrochloride
Example 263: 4-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}-N-(2,2,2-trifluoroethyl)benzamide
Example 264: 6-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}-1,3-benzoxazol-2(3H)-one
Example 265: (1S,2R)-1-(Methoxymethyl)-2-{5-phenyl-4-[((2R)-2-{2-[(3,5,6-trifluoropyridin-2-yl)oxy]ethyl}piperazin-1-yl)carbonyl]-1H-imidazol-1-yl}cyclohexanol dihydrochloride
Example 266: Methyl 5-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethoxy}pyridine-2-carboxylate dihydrochloride
Example 267: (1S,2R)-1-(Methoxymethyl)-2-{4-[((2R)-2-{2-[3-(5-methyl-1,3,4-oxadiazol-2-yl)phenoxy]ethyl}piperazin-1-yl)carbonyl]-5-phenyl-1H-imidazol-1-yl}cyclohexanol dihydrochloride
Example 268: (1S,2R)-2-{4-[((2R)-2-{2-[4-(4-Acetylpiperazin-1-yl)phenoxy]ethyl}piperazin-1-yl)carbonyl]-5-phenyl-1H-imidazol-1-yl}-1-(methoxymethyl)cyclohexanol dihydrochloride
Example 269: Ethyl 4-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethoxy}-2-methyl-1,3-thiazole-5-carboxylate hydrochloride
Example 270: Methyl 3-(4-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethoxy}phenyl)propionate
Example 271: 4-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}benzonitrile
Example 272: Methyl 3-fluoro-4-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethoxy}benzoate
Example 273: Methyl 2-fluoro-4-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethoxy}benzoate
Example 274: Methyl 3-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethoxy}pyridine-2-carboxylate dihydrochloride
Example 275: Ethyl 5-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethoxy}-1-methyl-1H-pyrazole-4-carboxylate hydrochloride
Example 276: Methyl (1-ethyl-3-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethoxy}-1H-pyrazol-4-yl)acetate hydrochloride
Example 277: (1S,2R)-2-[4-({(2R)-2-[2-({2-[(Dimethylamino)methyl]pyridin-3-yl}oxy)ethyl]piperazin-1-yl}carbonyl)-5-phenyl-1H-imidazol-1-yl]-1-(methoxymethyl)cyclohexanol trihydrochloride
Example 278: 7-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}-3,4-dihydroquinolin-2(1H)-one
Example 279: Methyl 3-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethoxy}thiophene-2-carboxylate hydrochloride
Example 280: 1-(3-Fluoro-4-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethoxy}phenyl)ethanone
Example 281: (1S,2R)-2-[4-({(2R)-2-[2-(4-Fluoro-2-methoxyphenoxy)ethyl]piperazin-1-yl}carbonyl)-5-phenyl-1H-imidazol-1-yl]-1-(methoxymethyl)cyclohexanol
Example 282: (1S,2R)-1-(Methoxymethyl)-2-[4-({(2R)-2-[2-(2-methoxy-4-methylphenoxy)ethyl]piperazin-1-yl}carbonyl)-5-phenyl-1H-imidazol-1-yl]cyclohexanol dihydrochloride
Example 283: 1-(4-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}-3-methoxyphenyl)ethanone dihydrochloride
Example 284: Ethyl 4-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethoxy}-3-methoxybenzoate dihydrochloride
Example 285: (1S,2R)-2-(4-{[(2R)-2-(2-{4-[(Dimethylamino)methyl]phenoxy}ethyl)piperazin-1-yl]carbonyl}-5-phenyl-1H-imidazol-1-yl)-1-(methoxymethyl)cyclohexanol
Example 286: (1S,2R)-2-(4-{[(2R)-2-(2-{4-[(Dimethylamino)methyl]-2-fluorophenoxy}ethyl)piperazin-1-yl]carbonyl}-5-phenyl-1H-imidazol-1-yl)-1-(methoxymethyl)cyclohexanol
Example 287: (1S,2R)-2-[4-({(2R)-2-[2-(2-Fluoro-6-methoxyphenoxy)ethyl]piperazin-1-yl}carbonyl)-5-phenyl-1H-imidazol-1-yl]-1-(methoxymethyl)cyclohexanol
Example 288: (1S,2R)-2-[4-({(2R)-2-[2-(4-Bromo-2-methoxyphenoxy)ethyl]piperazin-1-yl}carbonyl)-5-phenyl-1H-imidazol-1-yl]-1-(methoxymethyl)cyclohexanol hydrochloride
Example 289: (1S,2R)-2-[4-({(2R)-2-[2-(4-Chloro-2-methoxyphenoxy)ethyl]piperazin-1-yl}carbonyl)-5-phenyl-1H-imidazol-1-yl]-1-(methoxymethyl)cyclohexanol hydrochloride
Example 290: (1S,2R)-2-[4-({(2R)-2-[2-(2-Ethoxyphenoxy)ethyl]piperazin-1-yl}carbonyl)-5-phenyl-1H-imidazol-1-yl]-1-(methoxymethyl)cyclohexanol hydrochloride
Example 291: (1S,2R)-2-[4-({(2R)-2-[2-(2,3-Dimethoxyphenoxy)ethyl]piperazin-1-yl}carbonyl)-5-phenyl-1H-imidazol-1-yl]-1-(methoxymethyl)cyclohexanol hydrochloride
Example 292: (1S,2R)-2-[4-({(2R)-2-[2-(2,6-Dimethoxy-4-methylphenoxy)ethyl]piperazin-1-yl}carbonyl)-5-phenyl-1H-imidazol-1-yl]-1-(methoxymethyl)cyclohexanol hydrochloride
Example 293: 7-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}-6-methoxy-2H-chromen-2-one
Example 294: 1-(4-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}phenyl)pyrrolidin-2-one
Example 295: (1S,2R)-2-[4-({(2R)-2-[2-(2-Fluoro-4-methoxyphenoxy)ethyl]piperazin-1-yl}carbonyl)-5-phenyl-1H-imidazol-1-yl]-1-(methoxymethyl)cyclohexanol
Example 296: (1S,2R)-2-[4-({(2R)-2-[2-(5-Fluoro-2-methoxyphenoxy)ethyl]piperazin-1-yl}carbonyl)-5-phenyl-1H-imidazol-1-yl]-1-(methoxymethyl)cyclohexanol
Example 297: (1S,2R)-2-[4-({(2R)-2-[2-(2-Fluoro-4-methylphenoxy)ethyl]piperazin-1-yl}carbonyl)-5-phenyl-1H-imidazol-1-yl]-1-(methoxymethyl)cyclohexanol
Example 298: Methyl 2-fluoro-5-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethoxy}benzoate
Example 299: Methyl 3-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethoxy}-4-methoxybenzoate
Example 300: 5-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}-3,4-dihydroisoquinolin-1(2H)-one
Example 301: (1S,2R)-1-(Methoxymethyl)-2-[5-phenyl-4-({(2R)-2-[2-(thieno[3,2-b]pyridine-7-yloxy)ethyl]piperazin-1-yl}carbonyl)-1H-imidazol-1-yl]cyclohexanol dihydrochloride
Example 302: Ethyl (4-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethoxy}-3-methoxyphenyl)acetate dihydrochloride
Example 303: (1S,2R)-2-[4-({(2R)-2-[2-(2-Isopropoxyphenoxy)ethyl]piperazin-1-yl}carbonyl)-5-phenyl-1H-imidazol-1-yl]-1-(methoxymethyl)cyclohexanol hydrochloride
Example 304: (1S,2R)-2-[4-({(2R)-2-[2-(1,3-Benzodioxol-5-yloxy)ethyl]piperazin-1-yl}carbonyl)-5-phenyl-1H-imidazol-1-yl]-1-(methoxymethyl)cyclohexanol hydrochloride
Example 305: (1S,2R)-1-(Methoxymethyl)-2-{5-phenyl-4-[((2R)-2-{2-[(1-phenyl-1H-1,2,4-triazol-3-yl)oxy]ethyl}piperazin-1-yl)carbonyl]-1H-imidazol-1-yl}cyclohexanol hydrochloride
Example 306: (1S,2R)-2-{4-[((2R)-2-{2-[2-Fluoro-4-(5-methyl-1,3,4-oxadiazol-2-yl)phenoxy]ethyl}piperazin-1-yl)carbonyl]-5-phenyl-1H-imidazol-1-yl}-1-(methoxymethyl)cyclohexanol
Example 307: (1S,2R)-2-{4-[((2R)-2-{2-[3-Fluoro-4-(5-methyl-1,3,4-oxadiazol-2-yl)phenoxy]ethyl}piperazin-1-yl)carbonyl]-5-phenyl-1H-imidazol-1-yl}-1-(methoxymethyl)cyclohexanol
Example 308: (1S,2R)-2-{4-[((2R)-2-{2-[4-Fluoro-3-(5-methyl-1,3,4-oxadiazol-2-yl)phenoxy]ethyl}piperazin-1-yl)carbonyl]-5-phenyl-1H-imidazol-1-yl}-1-(methoxymethyl)cyclohexanol
Example 309: (1S,2R)-1-(Methoxymethyl)-2-{4-[((2R)-2-{2-[2-methoxy-4-(5-methyl-1,3,4-oxadiazol-2-yl)phenoxy]ethyl}piperazin-1-yl)carbonyl]-5-phenyl-1H-imidazol-1-yl}cyclohexanol
Example 310: (1S,2R)-1-(Methoxymethyl)-2-{4-[((2R)-2-{2-[2-methoxy-5-(5-methyl-1,3,4-oxadiazol-2-yl)phenoxy]ethyl}piperazin-1-yl)carbonyl]-5-phenyl-1H-imidazol-1-yl}cyclohexanol
Example 311: (1S,2R)-1-(Methoxymethyl)-2-[4-({(2R)-2-[2-(4-methyl-1H-pyrazol-1-yl)ethyl]piperazin-1-yl}carbonyl)-5-phenyl-1H-imidazol-1-yl]cyclohexanol dihydrochloride
Example 312: (1S,2R)-2-[4-({(2R)-2-[2-(1H-Benzimidazol-1-yl)ethyl]piperazin-1-yl}carbonyl)-5-phenyl-1H-imidazol-1-yl]-1-(methoxymethyl)cyclohexanol
Example 313: (1S,2R)-1-(Methoxymethyl)-2-{5-phenyl-4-[((2R)-2-{2-[3-(trifluoromethyl)-1H-pyrazol-1-yl]ethyl}piperazin-1-yl)carbonyl]-1H-imidazol-1-yl}cyclohexanol
Example 314: (1S,2R)-2-[4-({(2R)-2-[2-(1H-1,2,3-Benzotriazol-1-yl)ethyl]piperazin-1-yl}carbonyl)-5-phenyl-1H-imidazol-1-yl]-1-(methoxymethyl)cyclohexanol
Example 315: (1S,2R)-1-(Methoxymethyl)-2-[5-phenyl-4-({(2R)-2-[2-(3-phenyl-1H-pyrazol-1-yl)ethyl]piperazin-1-yl}carbonyl)-1H-imidazol-1-yl]cyclohexanol
Example 316: 4-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethyl}-2H-1,4-benzoxazin-3(4H)-one dihydrochloride
Example 317: 3-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethyl}-1,3-benzoxazol-2(3H)-one dihydrochloride
Example 318: Ethyl 1-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethyl}-3-methyl-1H-pyrazole-5-carboxylate
Example 319: Ethyl 1-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethyl}-5-methyl-1H-pyrazole-3-carboxylate
Example 320: (1S,2R)-1-(Methoxymethyl)-2-[5-phenyl-4-({2-[2-(4,5,6,7-tetrahydro-1H-indazol-1-yl)ethyl]piperazin-1-yl}carbonyl)-1H-imidazol-1-yl]cyclohexanol
Example 321: 1-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethyl}-3-methyl-1,3-dihydro-2H-benzimidazol-2-one
Example 322: Methyl 1-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethyl}-1H-indazole-4-carboxylate
Example 323: (1S,2R)-2-[4-({(2R)-2-[2-(3,5-Di-tert-butyl-1H-pyrazol-1-yl)ethyl]piperazin-1-yl}carbonyl)-5-phenyl-1H-imidazol-1-yl]-1-(methoxymethyl)cyclohexanol
Example 324: (1S,2R)-2-[4-({(2R)-2-[2-(1H-Indol-1-yl)ethyl]piperazin-1-yl}carbonyl)-5-phenyl-1H-imidazol-1-yl]-1-(methoxymethyl)cyclohexanol
Example 325: (1S,2R)-1-(Methoxymethyl)-2-[5-phenyl-4-({(2R)-2-[2-(2-phenyl-1H-imidazol-1-yl)ethyl]piperazin-1-yl}carbonyl)-1H-imidazol-1-yl]cyclohexanol
Example 326: (1S,2R)-2-[4-({(2R)-2-[2-(3,5-Dimethyl-1H-pyrazol-1-yl)ethyl]piperazin-1-yl}carbonyl)-5-phenyl-1H-imidazol-1-yl]-1-(methoxymethyl)cyclohexanol
Example 327: (1S,2R)-2-{4-[((2R)-2-{2-[4-(Hydroxymethyl)-1H-1,2,3-triazol-1-yl]ethyl}piperazin-1-yl)carbonyl]-5-phenyl-1H-imidazol-1-yl}-1-(methoxymethyl)cyclohexanol
Example 328: (1S,2R)-2-{4-[((2R)-2-{2-[4-(2-Hydroxyethyl)-1H-1,2,3-triazol-1-yl]ethyl}piperazin-1-yl)carbonyl]-5-phenyl-1H-imidazol-1-yl}-1-(methoxymethyl)cyclohexanol
Example 329: (1S,2R)-2-[4-({(2R)-2-[2-(4-Cyclopropyl-1H-1,2,3-triazol-1-yl)ethyl]piperazin-1-yl}carbonyl)-5-phenyl-1H-imidazol-1-yl]-1-(methoxymethyl)cyclohexanol
Example 330: Ethyl 1-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethyl}-1H-1,2,3-triazole-4-carboxylate
Example 331: Methyl 1-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethyl}-3,5-dimethyl-1H-pyrazole-4-carboxylate
Example 332: Ethyl 3-tert-butyl-1-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethyl}-1H-pyrazole-5-carboxylate
Example 333: 1-(1-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethyl}-1H-1,2,3-triazol-4-yl)ethanone
Example 334: Ethyl 1-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethyl}-1H-pyrazole-4-carboxylate
Example 335: Methyl 1-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethyl}-1H-pyrrole-3-carboxylate
Example 336: Ethyl 1-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethyl}-2-methyl-1H-pyrrole-3-carboxylate
Example 337: (1-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethyl}-1H-1,2,3-triazol-4-yl)methyl acetate dihydrochloride
Example 338: Methyl 1-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethyl}-1H-indazole-3-carboxylate
Example 339: Methyl 2-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethyl}-2H-indazole-3-carboxylate
Example 340: Ethyl 3-cyclopropyl-1-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethyl}-1H-pyrazole-5-carboxylate
Example 341: 1-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethyl}-1H-indole-3-carbonitrile
Example 342: Ethyl 1-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethyl}-1H-1,2,3-triazole-5-carboxylate
Example 343: Ethyl 2-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethyl}-2H-1,2,3-triazole-4-carboxylate

In the same manner as in Example 2 (Method B), the following compounds (Examples 344-347) were obtained.

### Example 344

### (2R)-2-Benzyl-1-[(1,5-dicyclohexyl-1H-imidazol-4-yl)carbonyl]piperazine

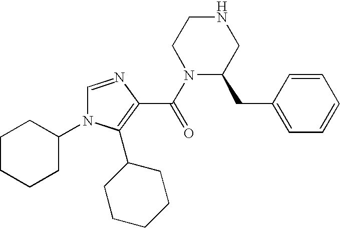

MS (ESI+, m/e) 435 (M+1)

### Example 345

### (2R)-2-Benzyl-1-[(1-cyclohexyl-5-cyclopropyl-1H-imidazol-4-yl)carbonyl]piperazine

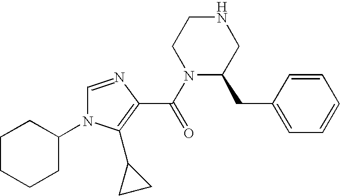

MS (ESI+, m/e) 393 (M+1)

### Example 346

### (2R)-2-Benzyl-1-[(1-cyclohexyl-2-ethoxy-5-phenyl-1H-imidazol-4-yl)carbonyl]piperazine

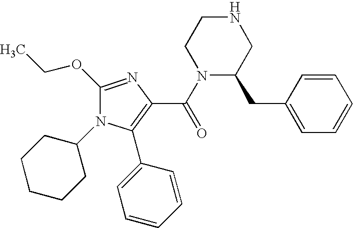

MS (ESI+, m/e) 473 (M+1)

### Example 347

### (2R)-2-Benzyl-1-[(2-chloro-1-cyclohexyl-5-phenyl-1H-imidazol-4-yl)carbonyl]piperazine

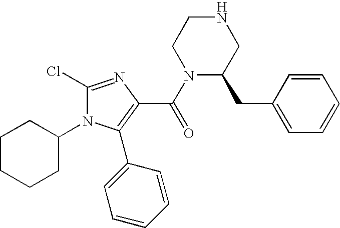

MS (ESI+, m/e) 465 (M+1)

In the same manner as in Example 6 (Method F) except that the final product was isolated as a hydrochloride by treating with 4N hydrogen chloride-ethyl acetate solution, the following compound (Example 348) was obtained.

### Example 348

### N-{[(2S)-1-({1-[2-(Ethoxymethyl)-2-hydroxycyclohexyl]-5-phenyl-1H-imidazol-4-yl}carbonyl)piperazin-2-yl]methyl}benzamide hydrochloride

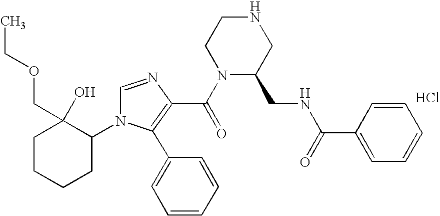

MS (ESI+, m/e) 546 (M+1)

### Example 349

### (2R)-2-Benzyl-1-[(1-cyclohexyl-5-phenyl-1H-imidazol-4-yl)carbonyl]piperazine

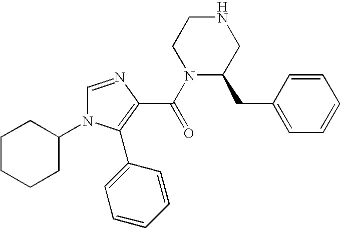

To tert-butyl (3R)-3-benzyl-4-[(1-cyclohexyl-5-phenyl-1H-imidazol-4-yl)carbonyl]piperazine-1-carboxylate (300 mg) was added TFA (3 ml), and the mixture was stirred at room temperature for 5 min, and poured into saturated aqueous sodium hydrogen carbonate. The mixture was extracted with ethyl acetate, and the extract was washed successively with water and saturated brine, and dried over anhydrous magnesium sulfate. The solvent was evaporated under reduced pressure to give the object compound (232 mg).

MS (ESI+, m/e) 429 (M+1)

### Example 350

### (1R,2R)-2-(4-{[(2R)-2-Benzylpiperazin-1-yl]carbonyl}-5-phenyl-1H-imidazol-1-yl)cyclopentanol and (1S,2S)-2-(4-{[(2R)-2-benzylpiperazin-1-yl]carbonyl}-5-phenyl-1H-imidazol-1-yl)cyclopentanol

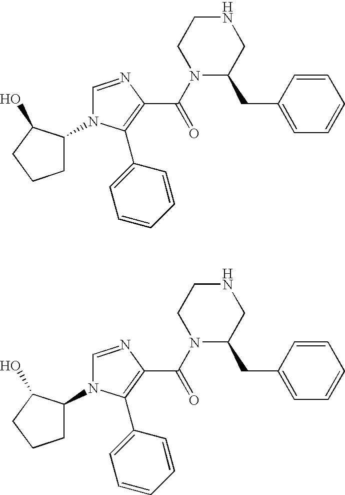

To tert-butyl (3R)-3-benzyl-4-{[1-(trans-2-hydroxycyclopentyl)-5-phenyl-1H-imidazol-4-yl]carbonyl}piperazine-1-carboxylate (1.15 g) was added TFA (10 ml), and the mixture was stirred at room temperature for 5 min, and poured into saturated aqueous sodium hydrogen carbonate. The mixture was extracted with ethyl acetate. The extract was washed with saturated brine, and dried over anhydrous magnesium sulfate, and the solvent was evaporated under reduced pressure. The residue was subjected to reversed-phase preparative HPLC (the purification conditions are described above). The object fraction was neutralized with saturated aqueous sodium hydrogen carbonate, and the mixture was extracted with ethyl acetate. The extract was dried over anhydrous sodium sulfate, and the solvent was evaporated under reduced pressure to give (1R,2R)-2-(4-{[(2R)-2-benzylpiperazin-1-yl]carbonyl}-5-phenyl-1H-imidazol-1-yl)cyclopentanol (96 mg) and (1S,2S)-2-(4-{[(2R)-2-benzylpiperazin-1-yl]carbonyl}-5-phenyl-1H-imidazol-1-yl)cyclopentanol (72 mg), as an amorphous solid, respectively.

MS (ESI+, m/e) 431 (M+1)

MS (ESI+, m/e) 431 (M+1)

### Example 351

### (1R,2R)-2-(4-{[(2R)-2-Isobutylpiperazin-1-yl]carbonyl}-5-phenyl-1H-imidazol-1-yl)cyclopentanol and (1S,2S)-2-(4-{[(2R)-2-isobutylpiperazin-1-yl]carbonyl}-5-phenyl-1H-imidazol-1-yl)cyclopentanol

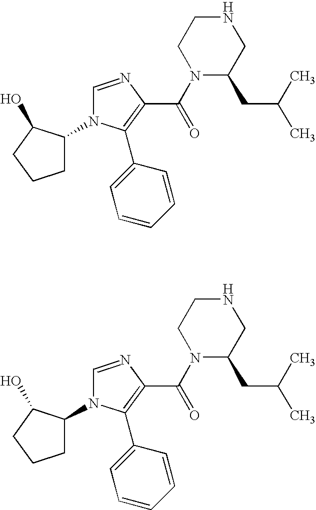

trans-2-(4-{[(2R)-4-Benzyl-2-isobutylpiperazin-1-yl]carbonyl}-5-phenyl-1H-imidazol-1-yl)cyclopentanol (270 mg) was dissolved in methanol (8 ml), 20% palladium hydroxide-carbon (50% containing water, 100 mg) was added thereto, and the mixture was subjected to catalytic reduction at ambient temperature and normal pressure for 12 hr. The catalyst was filtered off, and the filtrate was concentrated under reduced pressure. The residue was subjected to reversed-phase preparative HPLC (the purification conditions are described above). The object fractions were neutralized with saturated aqueous sodium hydrogen carbonate, and the mixtures were extracted with chloroform, respectively. The extracts were dried over anhydrous sodium sulfate, and the solvents were evaporated under reduced pressure, respectively. The residue of the less polar fraction was vacuum-dried to give (1S,2S)-2-(4-{[(2R)-2-isobutylpiperazin-1-yl]carbonyl}-5-phenyl-1H-imidazol-1-yl)cyclopentanol (60 mg), and the residue of the more polar fraction was vacuum-dried to give (1R,2R)-2-(4-{[(2R)-2-isobutylpiperazin-1-yl]carbonyl}-5-phenyl-1H-imidazol-1-yl)cyclopentanol (50 mg), as an amorphous solid, respectively.

MS (ESI+, m/e) 397 (M+1)

MS (ESI+, m/e) 397 (M+1)

### Example 352

### (1R,2S)-2-(4-{[(2R)-2-Benzylpiperazin-1-yl]carbonyl}-5-phenyl-1H-imidazol-1-yl)cyclohexanol

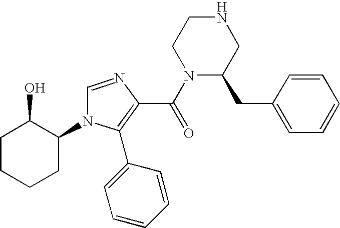

To tert-butyl (3R)-3-benzyl-4-({1-[(1S,2R)-2-hydroxycyclohexyl]-5-phenyl-1H-imidazol-4-yl}carbonyl)piperazine-1-carboxylate (110 mg) was added TFA (3 ml), and the mixture was stirred at room temperature for 5 min, and poured into saturated aqueous sodium hydrogen carbonate. The mixture was extracted with ethyl acetate, and the extract was washed successively with water and saturated brine, and dried over anhydrous magnesium sulfate. The solvent was evaporated under reduced pressure to give the object compound (92 mg).

MS (ESI+, m/e) 445 (M+1)

### Example 353

### (1S,2R)-2-(4-{[(2R)-2-Benzylpiperazin-1-yl]carbonyl}-5-phenyl-1H-imidazol-1-yl)cyclohexanol

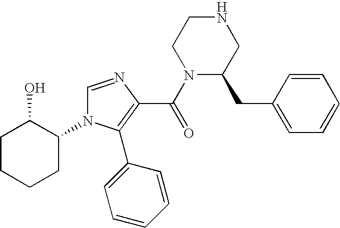

To tert-butyl (3R)-3-benzyl-4-{[1-(cis-2-hydroxycyclohexyl)-5-phenyl-1H-imidazol-4-yl]carbonyl}piperazine-1-carboxylate (260 mg) was added TFA (3 ml), and the mixture was stirred at room temperature for 5 min, and poured into saturated aqueous sodium hydrogen carbonate. The mixture was extracted with ethyl acetate. The extract was washed with saturated brine, and dried over anhydrous magnesium sulfate, and the solvent was evaporated under reduced pressure. The residue was subjected to reversed-phase preparative HPLC (the purification conditions are described above). The object fraction was neutralized with saturated aqueous sodium hydrogen carbonate, and the mixture was extracted with ethyl acetate. The extract was dried over anhydrous sodium sulfate, and the solvent was evaporated under reduced pressure to give the object compound (89 mg).

MS (ESI+, m/e) 445 (M+1) (The other diastereomer obtained by this method is the same as the compound of the above-mentioned Example 352.)

### Example 354

### [(1R,2S)-2-(4-{[(2R)-2-Benzylpiperazin-1-yl]carbonyl}-5-phenyl-1H-imidazol-1-yl)cyclohexyl]methanol, [(1S,2R)-2-(4-{[(2R)-2-benzylpiperazin-1-yl]carbonyl}-5-phenyl-1H-imidazol-1-yl)cyclohexyl]methanol, [(1R,2S)-2-(4-{[(2R)-2-benzylpiperazin-1-yl]carbonyl}-5-phenyl-1H-imidazol-1-yl)cyclohexyl]methyl acetate and [(1S,2R)-2-(4-{[(2R)-2-benzylpiperazin-1-yl]carbonyl}-5-phenyl-1H-imidazol-1-yl)cyclohexyl]methyl acetate

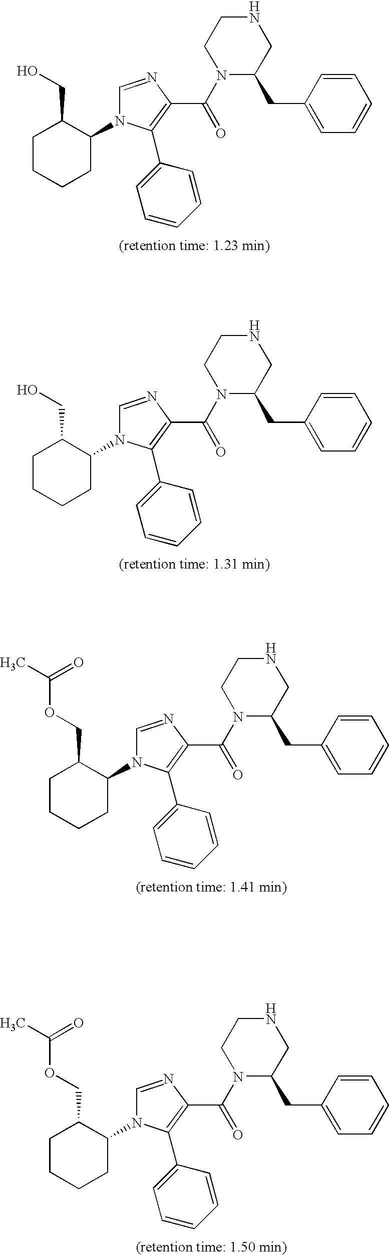

Methyl 1-[cis-2-(hydroxymethyl)cyclohexyl]-5-phenyl-1H-imidazole-4-carboxylate (containing a trace of ethyl acetate) (600 mg) and lithium hydroxide (120 mg) were dissolved in a mixed solvent of methanol (10 ml) and water (2 ml), and the solution was heated under reflux for 12 hr. The reaction mixture was concentrated under reduced pressure, and the residue was mixed with tert-butyl (3R)-3-benzylpiperazine-1-carboxylate (530 mg), WSC.HCl (440 mg), HOBt (2.90 g) and DMF (10 ml). The mixture was stirred at 60° C. for 3 hr, poured into aqueous potassium carbonate solution, and the mixture was extracted with ethyl acetate. The extract was washed with saturated brine, and dried over anhydrous magnesium sulfate, and the solvent was evaporated under reduced pressure. The residue was dissolved in TFA (5 ml). The solution was stirred for 30 min, and poured into aqueous potassium carbonate solution, and the mixture was extracted with dichloroethane. The extract was dried over anhydrous magnesium sulfate, and concentrated under reduced pressure. The residue was subjected to reversed-phase preparative HPLC (the purification conditions are described above). The object fractions were diluted with aqueous potassium carbonate solution, and the mixtures were extracted with ethyl acetate, respectively. The extracts were dried over anhydrous magnesium sulfate, and concentrated under reduced pressure to give the object compounds as an amorphous solid, respectively.
[(1R,2S)-2-(4-{[(2R)-2-Benzylpiperazin-1-yl]carbonyl}-5-phenyl-1H-imidazol-1-yl)cyclohexyl]methanol (46 mg): MS (ESI+, m/e) 459 (M+1), retention time 1.23 min
[(1S,2R)-2-(4-{[(2R)-2-Benzylpiperazin-1-yl]carbonyl}-5-phenyl-1H-imidazol-1-yl)cyclohexyl]methanol (42 mg): MS (ESI+, m/e) 459 (M+1), retention time 1.31 min
[(1R,2S)-2-(4-{[(2R)-2-Benzylpiperazin-1-yl]carbonyl}-5-phenyl-1H-imidazol-1-yl)cyclohexyl]methyl acetate (55 mg): MS (ESI+, m/e) 501 (M+1), retention time 1.41 min
[(1S,2R)-2-(4-{[(2R)-2-Benzylpiperazin-1-yl]carbonyl}-5-phenyl-1H-imidazol-1-yl)cyclohexyl]methyl acetate (74 mg): MS (ESI+, m/e) 501 (M+1), retention time 1.51 min
   (The above-mentioned “retention time” means retention time during LC/MS spectrum measurement under the aforementioned conditions.)

### Example 355

### trans-2-(4-{[(2R)-2-Benzylpiperazin-1-yl]carbonyl}-2-methyl-5-phenyl-1H-imidazol-1-yl)cyclohexanol trifluoroacetate

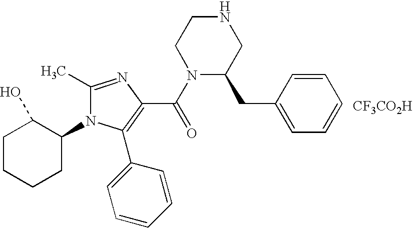

tert-Butyl (3R)-3-benzyl-4-({1-[trans-2-hydroxycyclohexyl]-2-methyl-5-phenyl-1H-imidazol-4-yl}carbonyl)piperazine-1-carboxylate (55 mg) was dissolved in 1,2-dichloroethane (2 ml), TFA (2 ml) was added, and the mixture was stirred at room temperature for 2 hr, and concentrated under reduced pressure. The residue was washed with diethyl ether to give the object compound (46 mg) as a TFA salt.

MS (ESI+, m/e) 459 (M+1)

### Example 356

### Ethyl (2S)-2-(4-{[(2R)-2-benzylpiperazin-1-yl]carbonyl}-5-phenyl-1H-imidazol-1-yl)cyclohexylidene]acetate hydrochloride

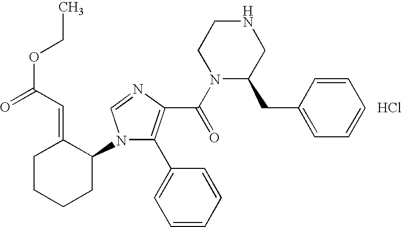

tert-Butyl (3R)-3-benzyl-4-({1-[(1S)-2-(2-ethoxy-2-oxoethylidene)cyclohexyl]-5-phenyl-1H-imidazol-4-yl}carbonyl)piperazine-1-carboxylate (100 mg) was dissolved in acetic acid-water (2:1, 1.5 ml), and the solution was stirred at 80° C. for 12 hr. The reaction mixture was poured into water, and the mixture was neutralized with aqueous sodium bicarbonate, and extracted with ethyl acetate-THF (1:1). The extract was dried over anhydrous sodium sulfate, and the solvent was evaporated under reduced pressure. The residue was subjected to reversed-phase preparative HPLC (the purification conditions are described above). The object fraction was neutralized with saturated aqueous sodium hydrogen carbonate, and the mixture was extracted with ethyl acetate. The extract was dried over anhydrous sodium sulfate, and 4N hydrogen chloride-ethyl acetate solution was added thereto. The solvent was evaporated under reduced pressure to give the object compound (70 mg) as an amorphous solid.

MS (ESI+, m/e) 513 (M+1)

### Example 357

### Ethyl [(1S,2S)-2-(4-{[(2R)-2-benzylpiperazin-1-yl]carbonyl}-5-phenyl-1H-imidazol-1-yl)cyclohexyl]carbamate

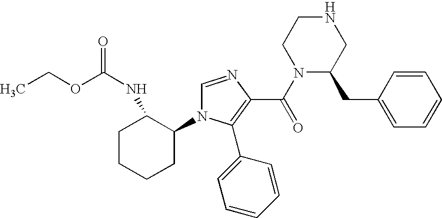

Ethyl [(1S,2S)-2-(4-{[(2R)-2,4-dibenzylpiperazin-1-yl]carbonyl}-5-phenyl-1H-imidazol-1-yl)cyclohexyl]carbamate (500 mg) was dissolved in methanol (10 ml), 20% palladium hydroxide-carbon (50% containing water, 100 mg) was added thereto, and the mixture was subjected to catalytic reduction at ambient temperature and normal pressure for 15 hr. The catalyst was filtered off, and the filtrate was concentrated under reduced pressure to give the object compound (420 mg) as an amorphous solid.

MS (ESI+, m/e) 516 (M+1)

### Example 358

### Ethyl [(1S,2S)-2-(4-{[(2R)-2-(3,5-difluorobenzyl)piperazin-1-yl]carbonyl}-5-phenyl-1H-imidazol-1-yl)cyclohexyl]carbamate

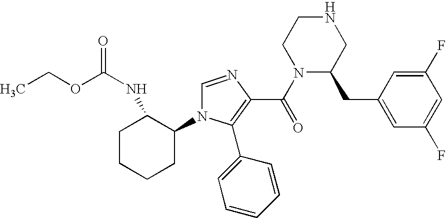

Ethyl [(1S,2S)-2-(4-{[(2R)-4-benzyl-2-(3,5-difluorobenzyl)piperazin-1-yl]carbonyl}-5-phenyl-1H-imidazol-1-yl)cyclohexyl]carbamate (530 mg) was dissolved in methanol (10 ml), 20% palladium hydroxide-carbon (50% containing water, 200 mg) was added thereto, and the mixture was subjected to catalytic reduction at ambient temperature and normal pressure for 15 hr. The catalyst was filtered off, and the filtrate was concentrated under reduced pressure to give the object compound (415 mg) as an amorphous solid.

MS (ESI+, m/e) 552 (M+1)

### Example 359

### Ethyl [(1S,2R)-2-(4-{[(2R)-2-benzylpiperazin-1-yl]carbonyl}-5-phenyl-1H-imidazol-1-yl)cyclohexyl]carbamate

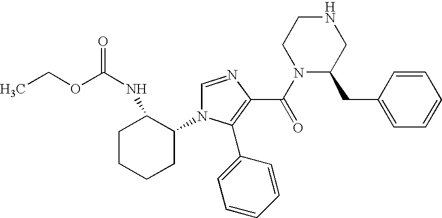

Ethyl 1-{cis-2-[(ethoxycarbonyl)amino]cyclohexyl}-5-phenyl-1H-imidazole-4-carboxylate (501 mg) was dissolved in ethanol-water (2:1, 6 ml), lithium hydroxide monohydrate (69 mg) was added, and the mixture was stirred at 65° C. for 3 hr. The reaction mixture was concentrated under reduced pressure, and the residue was suspended in ethanol. The suspension was again concentrated under reduced pressure, and the residue was vacuum-dried. This was suspended in DMF (5 ml), tert-butyl (3R)-3-benzylpiperazine-1-carboxylate (360 mg), WSC.HCl (498 mg) and HOBt (796 mg) were added, and the mixture was stirred at room temperature for 12 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate-hexane (3:7-7:3) was concentrated under reduced pressure to give tert-butyl (3R)-3-benzyl-4-[(1-{cis-2-[(ethoxycarbonyl)amino]cyclohexyl}-5-phenyl-1H-imidazol-4-yl)carbonyl]piperazine-1-carboxylate (560 mg) as an amorphous solid. 500 mg therefrom was dissolved in dichloromethane (1 ml), TFA (1 ml) was added at room temperature, and the mixture was stirred for 30 min. The reaction mixture was concentrated under reduced pressure, and the residue was neutralized with 6% aqueous sodium bicarbonate. The liberated oil was extracted with ethyl acetate. The extract was washed with saturated brine, and dried over anhydrous sodium sulfate, and the solvent was evaporated under reduced pressure. The residue was subjected to reversed-phase preparative HPLC (the purification conditions are described above). The object less polar fraction was neutralized with saturated aqueous sodium hydrogen carbonate, and the mixture was extracted with ethyl acetate. The extract was dried over anhydrous sodium sulfate, and the solvent was evaporated under reduced pressure to give the object compound (55 mg) as an amorphous solid.

MS (ESI+, m/e) 516 (M+1)

### Example 360

### 2-(4-{[(2R)-2-Benzylpiperazin-1-yl]carbonyl}-5-phenyl-1H-imidazol-1-yl)-1-butylcyclohexanol hydrochloride

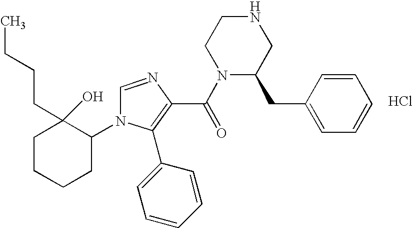

tert-Butyl (3R)-3-benzyl-4-{[1-(2-butyl-2-hydroxycyclohexyl)-5-phenyl-1H-imidazol-4-yl]carbonyl}piperazine-1-carboxylate (36 mg) was dissolved in ethyl acetate (0.5 ml), 4N hydrogen chloride-ethyl acetate solution (0.5 ml) was added, and the mixture was stirred at room temperature for 1 hr, and concentrated under reduced pressure to give the object compound (30 mg).

MS (ESI+, m/e) 501 (M+1)

### Example 361

### 2-(4-{[(2R)-2-Benzylpiperazin-1-yl]carbonyl}-5-phenyl-1H-imidazol-1-yl)-1-(ethoxymethyl)cyclohexanol

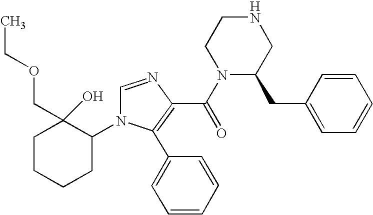

tert-Butyl (3R)-3-benzyl-4-{[1-(1-oxaspiro[2.5]oct-4-yl)-5-phenyl-1H-imidazol-4-yl]carbonyl}piperazine-1-carboxylate (170 mg) was dissolved in DMF (3 ml), sodium ethoxide (61 mg) was added, and the mixture was stirred at 60° C. for 15 hr. To the reaction mixture was added aqueous sodium bicarbonate, and the mixture was extracted with ethyl acetate. The extract was washed with saturated brine, dried over anhydrous magnesium sulfate, and concentrated under reduced pressure. The residue was subjected to silica gel column chromatography, and the fraction eluted with ethyl acetate-methanol (9:1) was concentrated under reduced pressure to give tert-butyl (3R)-3-benzyl-4-({1-[2-(ethoxymethyl)-2-hydroxycyclohexyl]-5-phenyl-1H-imidazol-4-yl}carbonyl)piperazine-1-carboxylate (70 mg) as an amorphous solid. The total amount thereof was dissolved in ethanol (2 ml), 4N hydrogen chloride-ethyl acetate solution (2 ml) was added, and the mixture was stirred at room temperature for 3 hr, and concentrated under reduced pressure. The residue was diluted with saturated aqueous sodium hydrogen carbonate, and the mixture was extracted with ethyl acetate. The extract was washed with saturated brine, dried over anhydrous magnesium sulfate, and concentrated under reduced pressure to give the object compound (30 mg) as an amorphous solid.

MS (ESI+, m/e) 503 (M+1)

In the same manner as in Example 361 except that the object compound was isolated as a hydrochloride, the following compound (Example 362) was obtained.

### Example 362

### 2-(4-{[(2R)-2-Benzylpiperazin-1-yl]carbonyl}-5-phenyl-1H-imidazol-1-yl)-1-(methoxymethyl)cyclohexanol hydrochloride

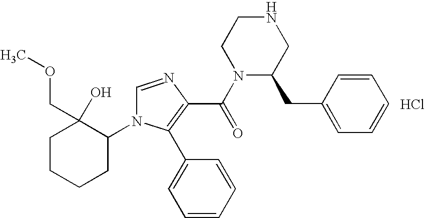

MS (ESI+, m/e) 489 (M+1)

### Example 363

### (1R,2R)-2-(4-{[(2R)-2-Benzylpiperazin-1-yl]carbonyl}-5-phenyl-1H-imidazol-1-yl)-1-(cyclopropylmethyl)cyclohexanol hydrochloride

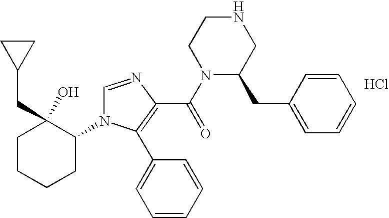

tert-Butyl (3R)-3-benzyl-4-({1-[(1R,2R)-2-(cyclopropylmethyl)-2-hydroxycyclohexyl]-5-phenyl-1H-imidazol-4-yl}carbonyl)piperazine-1-carboxylate (49 mg) was dissolved in methanol (2 ml), 4N hydrogen chloride-ethyl acetate solution (2 ml) was added, and the mixture was stirred at room temperature for 3 hr, and concentrated under reduced pressure. To the residue was toluene (5 ml) was added, and the mixture was further concentrated under reduced pressure to give the object compound (15 mg) as an amorphous solid.

MS (ESI+, m/e) 499 (M+1)

In the same manner as in Example 363, the following compound (Example 364) was obtained.

### Example 364

### (1S,2S)-2-(4-{[(2R)-2-Benzylpiperazin-1-yl]carbonyl}-5-phenyl-1H-imidazol-1-yl)-1-(cyclopropylmethyl)cyclohexanol hydrochloride

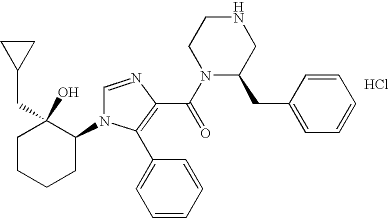

MS (ESI+, m/e) 499 (M+1)

### Example 365

### (1R,2R)-2-(4-{[(2R)-2-Benzylpiperazin-1-yl]carbonyl}-5-phenyl-1H-imidazol-1-yl)-1-({[2-(methylsulfonyl)ethyl]amino}methyl)cyclohexanol and (1S,2S)-2-(4-{[(2R)-2-benzylpiperazin-1-yl]carbonyl}-5-phenyl-1H-imidazol-1-yl)-1-({[2-(methylsulfonyl)ethyl]amino}methyl)cyclohexanol

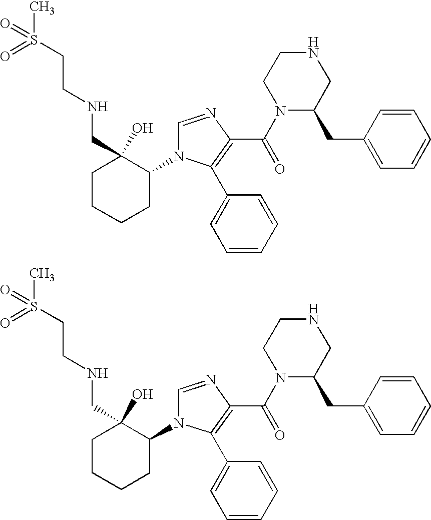

tert-Butyl (3R)-3-benzyl-4-{[1-(1-oxaspiro[2.5]oct-4-yl)-5-phenyl-1H-imidazol-4-yl]carbonyl}piperazine-1-carboxylate (221 mg) and 2-(methylsulfonyl)ethanamine (99 mg) were dissolved in acetonitrile (5 ml), lithium perchlorate (85 mg) was added, and the mixture was reacted at 100° C. for 5 min using microwave reactor. The reaction mixture was concentrated under reduced pressure. The residue was subjected to basic silica gel column chromatography, and the fraction eluted with ethyl acetate-methanol (4:1) was concentrated under reduced pressure to give tert-butyl (3R)-3-benzyl-4-({1-[2-hydroxy-2-({[2-(methylsulfonyl)ethyl]amino}methyl)cyclohexyl]-5-phenyl-1H-imidazol-4-yl}carbonyl)piperazine-1-carboxylate (235 mg) as an amorphous solid. To the total amount thereof was added 4N hydrogen chloride-ethyl acetate solution (2 ml), and the mixture was stirred at room temperature for 3 hr, and concentrated under reduced pressure. The residue was subjected to reversed-phase preparative HPLC (the purification conditions are described above). The object fractions were neutralized with saturated aqueous sodium hydrogen carbonate, and the mixtures were extracted with ethyl acetate, respectively. The extracts were dried over anhydrous sodium sulfate, and the solvents were evaporated under reduced pressure to give (1R,2R)-2-(4-{[(2R)-2-benzylpiperazin-1-yl]carbonyl}-5-phenyl-1H-imidazol-1-yl)-1-({[2-(methylsulfonyl)ethyl]amino}methyl)cyclohexanol (26 mg) as an amorphous solid, and (1S,2S)-2-(4-{[(2R)-2-benzylpiperazin-1-yl]carbonyl}-5-phenyl-1H-imidazol-1-yl)-1-({[2-(methylsulfonyl)ethyl]amino}methyl)cyclohexanol (15 mg) as an amorphous solid.

MS (ESI+, m/e) 580 (M+1)

MS (ESI+, m/e) 580 (M+1)

### Example 366

### Example 366a

### (1S,2R)-2-(4-{[(2R)-2-Benzylpiperazin-1-yl]carbonyl}-5-phenyl-1H-imidazol-1-yl)-1-(methoxymethyl)cyclohexanol hydrochloride

### Example 366b

### (1R,2S)-2-(4-{[(2R)-2-Benzylpiperazin-1-yl]carbonyl}-5-phenyl-1H-imidazol-1-yl)-1-(methoxymethyl)cyclohexanol hydrochloride

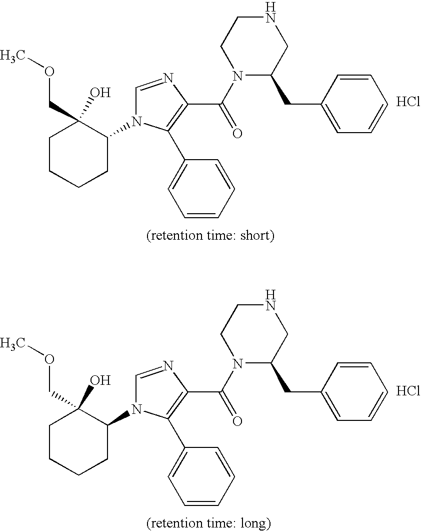

2-(4-{[(2R)-2-Benzylpiperazin-1-yl]carbonyl}-5-phenyl-1H-imidazol-1-yl)-1-(methoxymethyl)cyclohexanol hydrochloride (70 mg) was subjected to reversed-phase preparative HPLC (the purification conditions are described above), and the object fractions were collected, and partitioned between ethyl acetate and saturated aqueous sodium hydrogen carbonate, respectively. The organic layers were dried over anhydrous sodium sulfate, and the solvents were evaporated under reduced pressure, respectively. 4N Hydrogen chloride-ethyl acetate solutions (1 ml) were added to the residues, and the mixtures were concentrated under reduced pressure, respectively. Toluene (5 ml) was added to the residues, and the mixtures were again concentrated under reduced pressure to give (1S,2R)-2-(4-{[(2R)-2-benzylpiperazin-1-yl]carbonyl}-5-phenyl-1H-imidazol-1-yl)-1-(methoxymethyl)cyclohexanol hydrochloride (HPLC retention time: short, Example 366a, 24 mg) as an amorphous solid, and (1R,2S)-2-(4-{[(2R)-2-benzylpiperazin-1-yl]carbonyl}-5-phenyl-1H-imidazol-1-yl)-1-(methoxymethyl)cyclohexanol hydrochloride (HPLC retention time: long, Example 366b, 17 mg) as an amorphous solid.

MS (ESI+, m/e) 489 (M+1)

MS (ESI+, m/e) 489 (M+1)

### Example 367

(the alternative synthetic method of the above-mentioned Example 366a; The object compound was isolated as a dihydrochloride.)

### (1S,2R)-2-(4-{[(2R)-2-Benzylpiperazin-1-yl]carbonyl}-5-phenyl-1H-imidazol-1-yl)-1-(methoxymethyl)cyclohexanol dihydrochloride

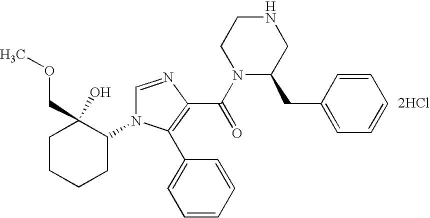

tert-Butyl (3R)-3-benzyl-4-({1-[(1R,2S)-2-hydroxy-2-(methoxymethyl)cyclohexyl]-5-phenyl-1H-imidazol-4-yl}carbonyl)piperazine-1-carboxylate (5.45 g) was dissolved in methanol (10 ml), 4N hydrogen chloride-ethyl acetate solution (10 ml) was added, and the mixture was stirred at room temperature for 3 hr, and concentrated under reduced pressure. To the residue was added saturated aqueous sodium hydrogen carbonate, and the mixture was extracted with ethyl acetate. The extract was dried over anhydrous magnesium sulfate, and concentrated under reduced pressure to give (1S,2R)-2-(4-{[(2R)-2-benzylpiperazin-1-yl]carbonyl}-5-phenyl-1H-imidazol-1-yl)-1-(methoxymethyl)cyclohexanol (4.47 g). 2.73 g therefrom was dissolved in ethanol (10 ml), 4N hydrogen chloride-ethyl acetate solution (3.07 ml) was added, and the mixture was heated with stirring to 70° C. Ethanol (5 ml) was added at the same temperature, and the mixture was cooled to room temperature while stirring. The precipitated crystals were collected by filtration to give the object compound (2.57 g).

MS (ESI+, m/e) 489 (M+1)

### Example 368

### (1S,2R)-2-(4-{[(2R)-2-Benzylpiperazin-1-yl]carbonyl}-5-phenyl-1H-imidazol-1-yl)-1-(methoxymethyl)cyclohexanol fumarate

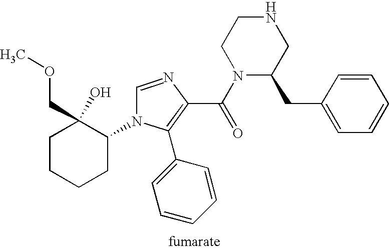

(1S,2R)-2-(4-{[(2R)-2-Benzylpiperazin-1-yl]carbonyl}-5-phenyl-1H-imidazol-1-yl)-1-(methoxymethyl)cyclohexanol obtained in the course of the above-mentioned Example 367 (1.00 g) was dissolved in ethyl acetate (20 ml), a solution of fumaric acid (238 mg) in ethanol (5 ml) was added, and the mixture was heated at 70° C. to give a homogeneous solution. Ethyl acetate (10 ml) was added at the same temperature, the mixture was left to stand at room temperature for 15 hr, and the precipitated crystals were collected by filtration to give the object compound (1.13 g).

MS (ESI+, m/e) 489 (M+1)

### Example 369

### (1R,2R)-2-(4-{[(2R)-2-Benzylpiperazin-1-yl]carbonyl}-5-phenyl-1H-imidazol-1-yl)-1-[2-(1H-tetrazol-5-yl)ethyl]cyclohexanol trifluoroacetate and (1S,2S)-2-(4-{[(2R)-2-benzylpiperazin-1-yl]carbonyl}-5-phenyl-1H-imidazol-1-yl)-1-[2-(1H-tetrazol-5-yl)ethyl]cyclohexanol trifluoroacetate

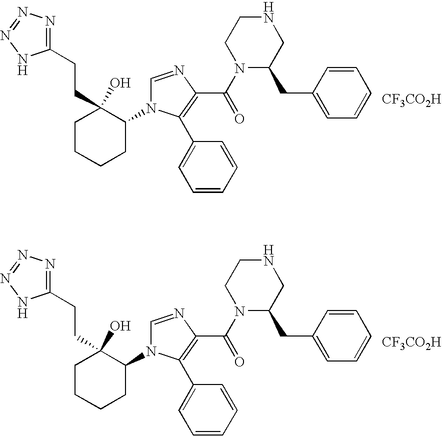

tert-Butyl (3R)-3-benzyl-4-({1-[2-(2-cyanoethyl)-2-hydroxycyclohexyl]-5-phenyl-1H-imidazol-4-yl}carbonyl)piperazine-1-carboxylate (150 mg) was dissolved in toluene (5 ml), and trimethylsilylazide (33 μl) and dibutyl(oxo)tin (6 mg) were added. The mixture was heated under reflux for 12 hr, and the solvent was evaporated under reduced pressure. To the residue was added saturated brine, and the mixture was extracted with ethyl acetate. The extract was dried over anhydrous magnesium sulfate, and concentrated under reduced pressure. The residue was subjected to silica gel column chromatography, and the fraction eluted with ethyl acetate-methanol (1:1) was concentrated under reduced pressure to give tert-butyl (3R)-3-benzyl-4-[(1-{2-hydroxy-2-[2-(1H-tetrazol-5-yl)ethyl]cyclohexyl}-5-phenyl-1H-imidazol-4-yl)carbonyl]piperazine-1-carboxylate (27 mg) as an amorphous solid. The total amount thereof was dissolved in methanol (1 ml), 4N hydrogen chloride-ethyl acetate solution (2 ml) was added, and the mixture was stirred at room temperature for 3 hr, and concentrated under reduced pressure. The residue was subjected to reversed-phase preparative HPLC (the purification conditions are described above), and the object fraction was concentrated under reduced pressure to give (1R,2R)-2-(4-{[(2R)-2-benzylpiperazin-1-yl]carbonyl}-5-phenyl-1H-imidazol-1-yl)-1-[2-(1H-tetrazol-5-yl)ethyl]cyclohexanol trifluoroacetate (6 mg) as an amorphous solid, and (1S,2S)-2-(4-{[(2R)-2-benzylpiperazin-1-yl]carbonyl}-5-phenyl-1H-imidazol-1-yl)-1-[2-(1H-tetrazol-5-yl)ethyl]cyclohexanol trifluoroacetate (9 mg) as an amorphous solid.

MS (ESI+, m/e) 541 (M+1)

MS (ESI+, m/e) 541 (M+1)

### Example 370

### N-{3-[(1R,2R)-2-(4-{[(2R)-2-Benzylpiperazin-1-yl]carbonyl}-5-phenyl-1H-imidazol-1-yl)-1-hydroxycyclohexyl]propyl}acetamide trifluoroacetate and N-{3-[(1S,2S)-2-(4-{[(2R)-2-benzylpiperazin-1-yl]carbonyl}-5-phenyl-1H-imidazol-1-yl)-1-hydroxycyclohexyl]propyl}acetamide trifluoroacetate

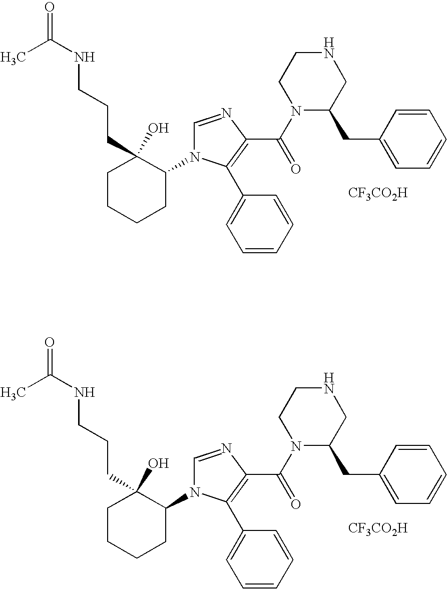

tert-Butyl (3R)-3-benzyl-4-({1-[2-(2-cyanoethyl)-2-hydroxycyclohexyl]-5-phenyl-1H-imidazol-4-yl}carbonyl)piperazine-1-carboxylate (150 mg) was dissolved in 1M ammonia-ethanol solution (15 ml), Raney cobalt (30 mg) was added thereto, and the mixture was subjected to catalytic reduction at ambient temperature and normal pressure for 15 hr. The catalyst was filtered off, and the filtrate was concentrated under reduced pressure to give tert-butyl (3R)-4-({1-[2-(3-aminopropyl)-2-hydroxycyclohexyl]-5-phenyl-1H-imidazol-4-yl}carbonyl)-3-benzylpiperazine-1-carboxylate (200 mg) as an oil. The total amount thereof was dissolved in pyridine (2 ml), and the solution was ice-cooled. Acetic anhydride (24 μl) was added, and the mixture was stirred at room temperature for 15 hr. To the reaction mixture was added aqueous sodium bicarbonate, and the mixture was extracted with ethyl acetate. The extract was washed with saturated brine, dried over anhydrous magnesium sulfate, and concentrated under reduced pressure. The residue was subjected to basic silica gel column chromatography, and the fraction eluted with ethyl acetate-methanol (9:1) was concentrated under reduced pressure to give tert-butyl (3R)-4-[(1-{2-[3-(acetylamino)propyl]-2-hydroxycyclohexyl}-5-phenyl-1H-imidazol-4-yl)carbonyl]-3-benzylpiperazine-1-carboxylate (32 mg) as an amorphous solid. The total amount thereof was dissolved in methanol (1 ml), 4N hydrogen chloride-ethyl acetate solution (2 ml) was added, and the mixture was stirred at room temperature for 3 hr, and concentrated under reduced pressure. The residue was subjected to reversed-phase preparative HPLC (the purification conditions are described above), and the object fraction was concentrated under reduced pressure to give N-{3-[(1R,2R)-2-(4-{[(2R)-2-benzylpiperazin-1-yl]carbonyl}-5-phenyl-1H-imidazol-1-yl)-1-hydroxycyclohexyl]propyl}acetamide trifluoroacetate (11 mg) as an amorphous solid, and N-{3-[(1S,2S)-2-(4-{[(2R)-2-benzylpiperazin-1-yl]carbonyl}-5-phenyl-1H-imidazol-1-yl)-1-hydroxycyclohexyl]propyl}acetamide trifluoroacetate (10 mg) as an amorphous solid.

MS (ESI+, m/e) 544 (M+1)

MS (ESI+, m/e) 544 (M+1)

### Example 371

### N-(2-{[(1S,2R)-2-(4-{[(2R)-2-Benzylpiperazin-1-yl]carbonyl}-5-phenyl-1H-imidazol-1-yl)-1-hydroxycyclohexyl]methoxy}ethyl)acetamide trifluoroacetate and N-(2-{[(1R,2S)-2-(4-{[(2R)-2-benzylpiperazin-1-yl]carbonyl}-5-phenyl-1H-imidazol-1-yl)-1-hydroxycyclohexyl]methoxy}ethyl)acetamide trifluoroacetate

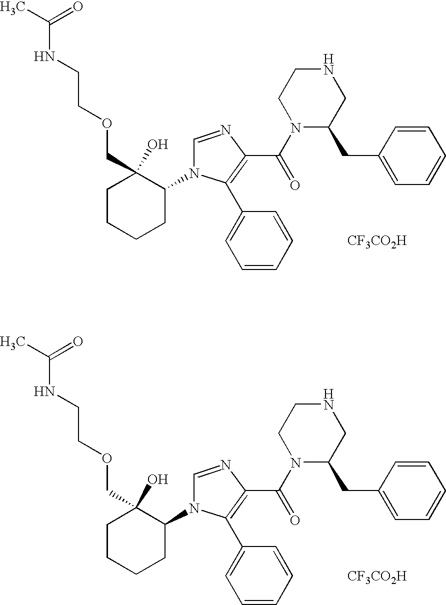

60% Sodium hydride (40 mg) was suspended in DMF (3 ml), N-(2-hydroxyethyl)acetamide (124 mg) was added, and the mixture was stirred at room temperature for 30 min. tert-Butyl (3R)-3-benzyl-4-{[1-(1-oxaspiro[2.5]oct-4-yl)-5-phenyl-1H-imidazol-4-yl]carbonyl}piperazine-1-carboxylate (111 mg) was added thereto, and the mixture was stirred at 60° C. for 15 hr. To the reaction mixture was added aqueous sodium bicarbonate, and the mixture was extracted with ethyl acetate. The extract was washed with saturated brine, dried over anhydrous magnesium sulfate, and concentrated under reduced pressure. The residue was subjected to silica gel column chromatography, and the fraction eluted with ethyl acetate-methanol (9:1) was concentrated under reduced pressure to give tert-butyl (3R)-4-{[1-(2-{[2-(acetylamino)ethoxy]methyl}-2-hydroxycyclohexyl)-5-phenyl-1H-imidazol-4-yl]carbonyl}-3-benzylpiperazine-1-carboxylate (79 mg) as an amorphous solid. To the total amount thereof was added 4N hydrogen chloride-ethyl acetate solution (2 ml), and the mixture was stirred at room temperature for 3 hr, and concentrated under reduced pressure. The residue was subjected to reversed-phase preparative HPLC (the purification conditions are described above), and the object fraction was concentrated under reduced pressure to give N-(2-{[(1R,2S)-2-(4-{[(2R)-2-benzylpiperazin-1-yl]carbonyl}-5-phenyl-1H-imidazol-1-yl)-1-hydroxycyclohexyl]methoxy}ethyl)acetamide trifluoroacetate (32 mg) as an amorphous solid, and N-(2-{[(1S,2R)-2-(4-{[(2R)-2-benzylpiperazin-1-yl]carbonyl}-5-phenyl-1H-imidazol-1-yl)-1-hydroxycyclohexyl]methoxy}ethyl)acetamide trifluoroacetate (37 mg) as an amorphous solid.

MS (ESI+, m/e) 560 (M+1)

MS (ESI+, m/e) 560 (M+1)

In the same manner as in Example 371, the following compound (Example 372) was obtained.

### Example 372

### (1S,2R)-2-(4-{[(2R)-2-Benzylpiperazin-1-yl]carbonyl}-5-phenyl-1H-imidazol-1-yl)-1-{[(1-methylpiperidin-4-yl)oxy]methyl}cyclohexanol trifluoroacetate and (1R,2S)-2-(4-{[(2R)-2-benzylpiperazin-1-yl]carbonyl}-5-phenyl-1H-imidazol-1-yl)-1-{[(1-methylpiperidin-4-yl)oxy]methyl}cyclohexanol trifluoroacetate

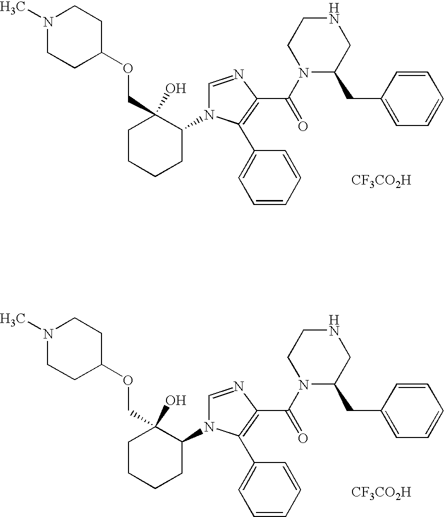

MS (ESI+, m/e) 572 (M+1)

MS (ESI+, m/e) 572 (M+1)

### Example 373

### (1S,2R)-2-(4-{[(2R)-2-Benzylpiperazin-1-yl]carbonyl}-5-phenyl-1H-imidazol-1-yl)-1-{[(1,1-dioxidotetrahydro-2H-thiopyran-4-yl)oxy]methyl}cyclohexanol and (1R,2S)-2-(4-{[(2R)-2-benzylpiperazin-1-yl]carbonyl}-5-phenyl-1H-imidazol-1-yl)-1-{[(1,1-dioxidotetrahydro-2H-thiopyran-4-yl)oxy]methyl}cyclohexanol

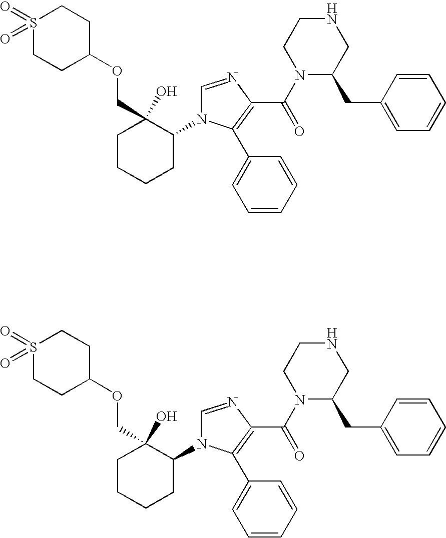

tert-Butyl (3R)-3-benzyl-4-{[1-(2-{[(1,1-dioxidotetrahydro-2H-thiopyran-4-yl)oxy]methyl}-2-hydroxycyclohexyl)-5-phenyl-1H-imidazol-4-yl]carbonyl}piperazine-1-carboxylate (74 mg) was dissolved in methanol (1 ml), 4N hydrogen chloride-ethyl acetate solution (2 ml) was added, and the mixture was stirred at room temperature for 3 hr, and concentrated under reduced pressure. The residue was subjected to reversed-phase preparative HPLC (the purification conditions are described above). The object fractions were collected, saturated aqueous sodium hydrogen carbonates were added, and the mixtures were extracted with ethyl acetate, respectively. The extracts were dried over anhydrous magnesium sulfate, and concentrated under reduced pressure to give (1S,2R)-2-(4-{[(2R)-2-benzylpiperazin-1-yl]carbonyl}-5-phenyl-1H-imidazol-1-yl)-1-{[(1,1-dioxidotetrahydro-2H-thiopyran-4-yl)oxy]methyl}cyclohexanol (31 mg) as an amorphous solid, and (1R,2S)-2-(4-{[(2R)-2-benzylpiperazin-1-yl]carbonyl}-5-phenyl-1H-imidazol-1-yl)-1-{[(1,1-dioxidotetrahydro-2H-thiopyran-4-yl)oxy]methyl}cyclohexanol (30 mg) as an amorphous solid.

MS (ESI+, m/e) 607 (M+1)

MS (ESI+, m/e) 607 (M+1)

### Example 374

### (1R,2S)-2-(4-{[(2R)-2-Benzylpiperazin-1-yl]carbonyl}-5-phenyl-1H-imidazol-1-yl)-1-{[1-(1,3-thiazol-2-yl)ethoxy]methyl}cyclohexanol hydrochloride and (1S,2R)-2-(4-{[(2R)-2-benzylpiperazin-1-yl]carbonyl}-5-phenyl-1H-imidazol-1-yl)-1-{[1-(1,3-thiazol-2-yl)ethoxy]methyl}cyclohexanol hydrochloride

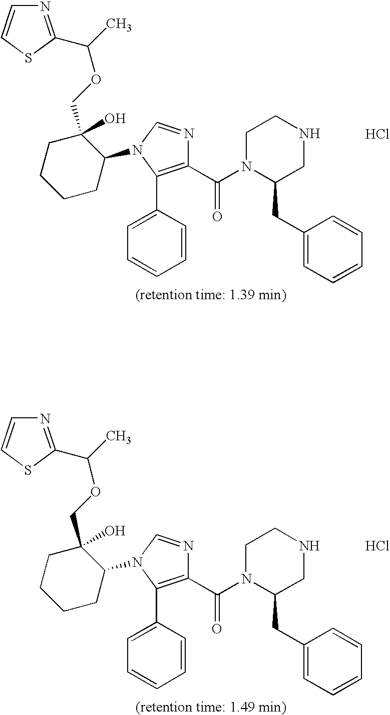

1-(1,3-Thiazol-2-yl)ethanol (230 mg) was dissolved in DMF (10 ml), and the solution was ice-cooled. Sodium hydride (60% in oil, 70 mg) was added thereto, and then tert-butyl (3R)-3-benzyl-4-{[1-(1-oxaspiro[2.5]oct-4-yl)-5-phenyl-1H-imidazol-4-yl]carbonyl}piperazine-1-carboxylate (200 mg) was added, and the mixture was stirred at 50° C. for 15 hr. The reaction mixture was poured into ice water, and the mixture was extracted with ethyl acetate. The extract was washed with saturated brine, and dried over anhydrous magnesium sulfate, and the solvent was evaporated under reduced pressure. The residue was dissolved in ethyl acetate (2.5 ml), 4N hydrogen chloride-ethyl acetate solution (2.5 ml) was added, and the mixture was stirred for 30 min, and concentrated under reduced pressure. The residue was subjected to reversed-phase preparative HPLC (the purification conditions are described above). The object fractions were collected, and diluted with aqueous potassium carbonate solution, and the mixtures were extracted with ethyl acetate, respectively. The extracts were dried over anhydrous magnesium sulfate, and concentrated under reduced pressure, respectively. The residues were treated with 4N hydrogen chloride-ethyl acetate solution to give the object compound, respectively.
(1R,2S)-2-(4-{[(2R)-2-Benzylpiperazin-1-yl]carbonyl}-5-phenyl-1H-imidazol-1-yl)-1-{[1-(1,3-thiazol-2-yl)ethoxy]methyl}cyclohexanol hydrochloride (32 mg): MS (ESI+, m/e) 586 (M+1), retention time 1.39 min
(1S,2R)-2-(4-{[(2R)-2-Benzylpiperazin-1-yl]carbonyl}-5-phenyl-1H-imidazol-1-yl)-1-{[1-(1,3-thiazol-2-yl)ethoxy]methyl}cyclohexanol hydrochloride (41 mg): MS (ESI+, m/e) 586 (M+1), retention time 1.49 min
   (The above-mentioned “retention time” means retention time during LC/MS spectrum measurement under the aforementioned conditions.)

In the same manner as in Example 374, the following compound (Example 375) was obtained.

### Example 375

### (1R,2S)-2-(4-{[(2R)-2-Benzylpiperazin-1-yl]carbonyl}-5-phenyl-1H-imidazol-1-yl)-1-{[1-methyl-1-(1,3-thiazol-2-yl)ethoxy]methyl}cyclohexanol dihydrochloride and (1S,2R)-2-(4-{[(2R)-2-benzylpiperazin-1-yl]carbonyl}-5-phenyl-1H-imidazol-1-yl)-1-{[1-methyl-1-(1,3-thiazol-2-yl)ethoxy]methyl}cyclohexanol dihydrochloride

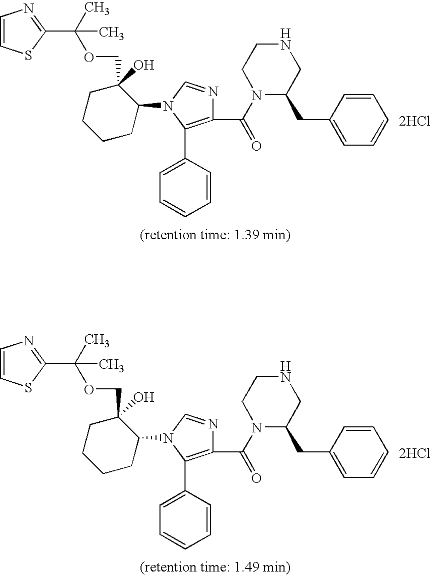

MS (ESI+, m/e) 600 (M+1)

MS (ESI+, m/e) 600 (M+1)

### Example 376

### 2-(4-{[(2R)-2-Benzylpiperazin-1-yl]carbonyl}-5-phenyl-1H-imidazol-1-yl)-1-(hydroxymethyl)cyclohexanol hydrochloride

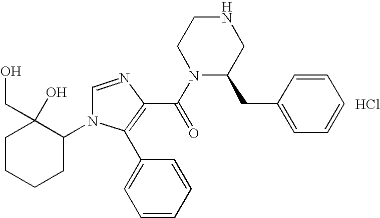

tert-Butyl (3R)-3-benzyl-4-{[1-(1-oxaspiro[2.5]oct-4-yl)-5-phenyl-1H-imidazol-4-yl]carbonyl}piperazine-1-carboxylate (111 mg) was dissolved in DMF (3 ml), lithium hydroxide monohydrate (84 mg) was added, and the mixture was stirred at 100° C. for 15 hr. To the reaction mixture was added aqueous sodium bicarbonate, and the mixture was extracted with ethyl acetate. The extract was washed with saturated brine, dried over anhydrous magnesium sulfate, and concentrated under reduced pressure. The residue was subjected to basic silica gel column chromatography, and the fraction eluted with ethyl acetate-methanol (9:1) was concentrated under reduced pressure to give tert-butyl (3R)-3-benzyl-4-({1-[2-hydroxy-2-(hydroxymethyl)cyclohexyl]-5-phenyl-1H-imidazol-4-yl}carbonyl)piperazine-1-carboxylate (70 mg) as an amorphous solid. The total amount thereof was dissolved in methanol (1 ml), 5% hydrogen chloride-methanol solution (1 ml) was added, and the mixture was stirred at room temperature for 15 hr. The reaction mixture was concentrated under reduced pressure, to the residue was added toluene, and the mixture was again concentrated under reduced pressure to give the object compound (60 mg) as an amorphous solid.

MS (ESI+, m/e) 475 (M+1)

### Example 377

### 2-(4-{[(2R)-2-Benzylpiperazin-1-yl]carbonyl}-5-phenyl-1H-imidazol-1-yl)-1-[(3-hydroxypropoxy)methyl]cyclohexanol dihydrochloride

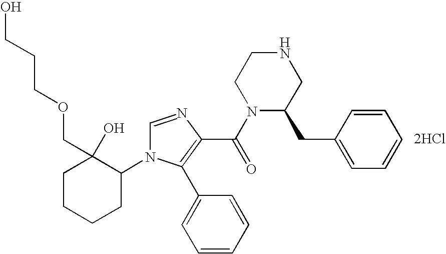

Sodium hydride (60% in oil) (40 mg) was suspended in DMF (3 ml), propane-1,3-diol (91 mg) was added, and the mixture was stirred at room temperature for 30 min. tert-Butyl (3R)-3-benzyl-4-{[1-(1-oxaspiro[2.5]oct-4-yl)-5-phenyl-1H-imidazol-4-yl]carbonyl}piperazine-1-carboxylate (110 mg) was added thereto, and the mixture was stirred at 60° C. for 15 hr. To the reaction mixture was added aqueous sodium bicarbonate, and the mixture was extracted with ethyl acetate. The extract was washed with saturated brine, dried over anhydrous magnesium sulfate, and concentrated under reduced pressure. The residue was subjected to silica gel column chromatography, and the fraction eluted with ethyl acetate-methanol (9:1) was concentrated under reduced pressure to give tert-butyl (3R)-3-benzyl-4-[(1-{2-hydroxy-2-[(3-hydroxypropoxy)methyl]cyclohexyl}-5-phenyl-1H-imidazol-4-yl)carbonyl]piperazine-1-carboxylate (91 mg) as an amorphous solid. The total amount thereof was dissolved in ethanol (2 ml), 5% hydrogen chloride-methanol solution (2 ml) was added, and the mixture was stirred at room temperature for 3 hr, and concentrated under reduced pressure. To the residue was added toluene (5 ml), and the mixture was again concentrated under reduced pressure to give the object compound (100 mg) as an amorphous solid.

MS (ESI+, m/e) 533 (M+1)

### Example 378

### (1R,2S)-2-(4-{[(2R)-2-Benzylpiperazin-1-yl]carbonyl}-5-phenyl-1H-imidazol-1-yl)-1-{[3-(methylthio)propoxy]methyl}cyclohexanol

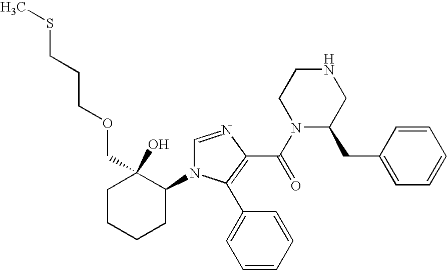

Ethyl 1-((1S,2R)-2-hydroxy-2-{[3-(methylthio)propoxy]methyl}cyclohexyl)-5-phenyl-1H-imidazole-4-carboxylate (318 mg) was dissolved in ethanol-THF (1:1, 4 ml), lithium hydroxide monohydrate (23 mg) and water (1 ml) were added thereto, and the mixture was stirred at 80° C. for 2 hr. The reaction mixture was concentrated under reduced pressure, and the residue was suspended in ethanol. The suspension was again concentrated under reduced pressure, and the residue was vacuum-dried. The half amount of the residue was suspended in DMF (5 ml), tert-butyl (3R)-3-benzylpiperazine-1-carboxylate (153 mg), WSC.HCl (142 mg) and HOBt (113 mg) were added, and the mixture was stirred at room temperature for 12 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate was concentrated under reduced pressure to give tert-butyl (3R)-3-benzyl-4-{[1-((1S,2R)-2-hydroxy-2-{[3-(methylthio)propoxy]methyl}cyclohexyl)-5-phenyl-1H-imidazol-4-yl]carbonyl}piperazine-1-carboxylate (94 mg) as an amorphous solid. The total amount thereof was dissolved in methanol (2 ml), and 4N hydrogen chloride-ethyl acetate solution (2 ml) was added thereto. The mixture was stirred at room temperature for 3 hr, and concentrated under reduced pressure. The residue was subjected to reversed-phase preparative HPLC (the purification conditions are described above), and the object fraction was partitioned between ethyl acetate and saturated aqueous sodium hydrogen carbonate. The organic layer was dried over anhydrous sodium sulfate, and the solvent was evaporated under reduced pressure to give the object compound (60 mg) as an amorphous solid.

MS (ESI+, m/e) 563 (M+1)

### Example 379

### (1S,2R)-2-(4-{[(2R)-2-(3,5-Difluorobenzyl)piperazin-1-yl]carbonyl}-5-phenyl-1H-imidazol-1-yl)-1-(methoxymethyl)cyclohexanol hydrochloride

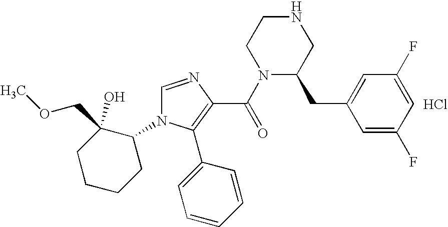

A solution of 1-[(1R,2S)-2-hydroxy-2-(methoxymethyl)cyclohexyl]-5-phenyl-1H-imidazole-4-carboxylic acid (569 mg), (3R)-1-benzyl-3-(3,5-difluorobenzyl)piperazine (496 mg), WSC.HCl (377 mg) and HOBt (266 mg) in DMF (9 ml) was stirred at room temperature for 15 hr, and poured into saturated aqueous sodium hydrogen carbonate, and the mixture was extracted with ethyl acetate. The extract was washed successively with water and saturated brine, and dried over anhydrous magnesium sulfate, and the solvent was evaporated under reduced pressure. The residue was subjected to basic silica gel column chromatography, and the fraction eluted with ethyl acetate-hexane (1.5:1-2:1) was concentrated under reduced pressure to give (1S,2R)-2-(4-{[(2R)-4-benzyl-2-(3,5-difluorobenzyl)piperazin-1-yl]carbonyl}-5-phenyl-1H-imidazol-1-yl)-1-(methoxymethyl)cyclohexanol (546 mg) as an amorphous solid. The total amount thereof was dissolved in methanol (15 ml), 20% palladium hydroxide-carbon (50% containing water, 275 mg) was added thereto, and the mixture was subjected to catalytic reduction at ambient temperature and normal pressure for 15 hr. The catalyst was filtered off, and the filtrate was concentrated under reduced pressure. The residue was subjected to basic silica gel column chromatography, and the fraction eluted with ethyl acetate-chloroform-methanol (1:1:0-10:10:1) was concentrated under reduced pressure. The residue was diluted with diethyl ether (6 ml), and 4N hydrogen chloride-ethyl acetate solution (244 μl) was added thereto. The precipitated crystals were collected by filtration to give the object compound (366 mg).

MS (ESI+, m/e) 525 (M+1)

### Example 380

### (1S,2R)-1-(Methoxymethyl)-2-(5-phenyl-4-{[(2R)-2-(pyridin-3-ylmethyl)piperazin-1-yl]carbonyl}-1H-imidazol-1-yl)cyclohexanol dihydrochloride

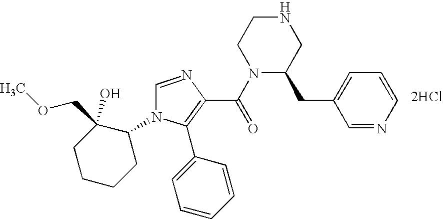

A solution of 1-[(1R,2S)-2-hydroxy-2-(methoxymethyl)cyclohexyl]-5-phenyl-1H-imidazole-4-carboxylic acid (132 mg), (3R)-1-benzyl-3-(pyridin-3-ylmethyl)piperazine (112 mg), WSC.HCl (92 mg) and HOBt (65 mg) in DMF (2.5 ml) was stirred at room temperature for 15 hr, and poured into saturated aqueous sodium hydrogen carbonate, and the mixture was extracted with ethyl acetate. The extract was washed successively with water and saturated brine, and dried over anhydrous magnesium sulfate, and the solvent was evaporated under reduced pressure. The residue was subjected to basic silica gel column chromatography, and the fraction eluted with ethyl acetate-methanol (1:0-20:1) was concentrated under reduced pressure to give (1S,2R)-2-(4-{[(2R)-4-benzyl-2-(pyridin-3-ylmethyl)piperazin-1-yl]carbonyl}-5-phenyl-1H-imidazol-1-yl)-1-(methoxymethyl)cyclohexanol (202 mg) as an amorphous solid. The total amount thereof was dissolved in methanol (5.5 ml), 20% palladium hydroxide-carbon (50% containing water, 100 mg) was added thereto, and the mixture was subjected to catalytic reduction at ambient temperature and normal pressure for 15 hr. The catalyst was filtered off, and the filtrate was concentrated under reduced pressure. The residue was subjected to reversed-phase preparative HPLC (the purification conditions are described above). The object fractions were collected, and diluted with saturated aqueous sodium hydrogen carbonate-saturated brine (1:1), and the mixture was extracted with chloroform. The extract was washed with saturated brine, and dried over anhydrous magnesium sulfate, and the solvent was evaporated under reduced pressure. The residue was diluted with diethyl ether (6 ml), and 4N hydrogen chloride-ethyl acetate solution (192 μl) was added thereto. The precipitated crystals were collected by filtration to give the object compound (103 mg).

MS (ESI+, m/e) 490 (M+1)

### Example 381

### (1S,2R)-2-(4-{[(2R)-2-(1H-Imidazol-4-ylmethyl)piperazin-1-yl]carbonyl}-5-phenyl-1H-imidazol-1-yl)-1-(methoxymethyl)cyclohexanol hydrochloride

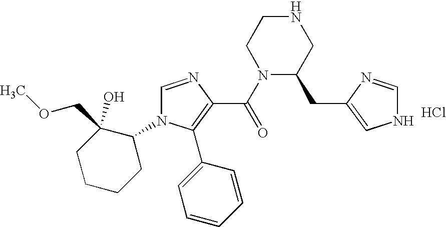

A solution of 1-[(1R,2S)-2-hydroxy-2-(methoxymethyl)cyclohexyl]-5-phenyl-1H-imidazole-4-carboxylic acid (132 mg), (3R)-1-benzyl-3-(1H-imidazol-4-ylmethyl)piperazine (108 mg), WSC.HCl (92 mg) and HOBt (65 mg) in DMF (2.5 ml) was stirred at room temperature for 15 hr, and poured into saturated aqueous sodium hydrogen carbonate, and the mixture was extracted with ethyl acetate. The extract was washed successively with water and saturated brine, and dried over anhydrous magnesium sulfate, and the solvent was evaporated under reduced pressure. The residue was subjected to basic silica gel column chromatography, and the fraction eluted with ethyl acetate-methanol (1:0-10:1) was concentrated under reduced pressure to give (1S,2R)-2-(4-{[(2R)-4-benzyl-2-(1H-imidazol-4-ylmethyl)piperazin-1-yl]carbonyl}-5-phenyl-1H-imidazol-1-yl)-1-(methoxymethyl)cyclohexanol (140 mg) as an amorphous solid. The total amount thereof was dissolved in methanol (10 ml), 20% palladium hydroxide-carbon (50% containing water, 140 mg) was added thereto, and the mixture was subjected to catalytic reduction at 60° C. for 10 hr under moderate-pressure (5 kgf/cm²). The catalyst was filtered off, and the filtrate was concentrated under reduced pressure. The residue was subjected to reversed-phase preparative HPLC (the purification conditions are described above). The object fraction was diluted with saturated aqueous sodium hydrogen carbonate-saturated brine (1:1), and the mixture was extracted with chloroform. The extract was washed with saturated brine, and dried over anhydrous magnesium sulfate, and the solvent was evaporated under reduced pressure. The residue was diluted with diethyl ether (2 ml), and 4N hydrogen chloride-ethyl acetate solution (68 μl) was added thereto. The precipitated crystals were collected by filtration to give the object compound (37 mg).

MS (ESI+, m/e) 479 (M+1)

### Example 382

### (1S,2R)-1-(Methoxymethyl)-2-(5-phenyl-4-{[(2R)-2-(3-phenylpropyl)piperazin-1-yl]carbonyl}-1H-imidazol-1-yl)cyclohexanol hydrochloride

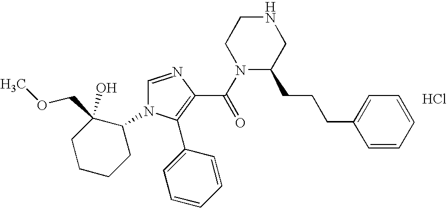

A solution of 1-[(1R,2S)-2-hydroxy-2-(methoxymethyl)cyclohexyl]-5-phenyl-1H-imidazole-4-carboxylic acid (198 mg), (3R)-1-benzyl-3-[(2E)-3-phenyl-2-propen-1-yl]piperazine (184 mg), WSC.HCl (138 mg) and HOBt (97 mg) in DMF (4 ml) was stirred at room temperature for 15 hr, and poured into saturated aqueous sodium hydrogen carbonate, and the mixture was extracted with ethyl acetate. The extract was washed successively with water and saturated brine, and dried over anhydrous magnesium sulfate, and the solvent was evaporated under reduced pressure. The residue was subjected to basic silica gel column chromatography, and the fraction eluted with ethyl acetate-hexane-methanol (1:1:0-20:0:1) was concentrated under reduced pressure to give (1S,2R)-2-[4-({(2R)-4-benzyl-2-[(2E)-3-phenyl-2-propen-1-yl]piperazin-1-yl}carbonyl)-5-phenyl-1H-imidazol-1-yl]-1-(methoxymethyl)cyclohexanol (301 mg) as an amorphous solid. The total amount thereof was dissolved in methanol (8.5 ml), 20% palladium hydroxide-carbon (50% containing water, 150 mg) was added thereto, and the mixture was subjected to catalytic reduction at ambient temperature and normal pressure for 15 hr. The catalyst was filtered off, and the filtrate was concentrated under reduced pressure. The residue was subjected to basic silica gel column chromatography, and the fraction eluted with ethyl acetate-methanol (25:1) was concentrated under reduced pressure. The residue was diluted with diethyl ether (3 ml), and 4N hydrogen chloride-ethyl acetate solution (137 μl) was added thereto. The precipitated crystals were collected by filtration to give the object compound (175 mg).

MS (ESI+, m/e) 517 (M+1)

### Example 383

### (1S,2R)-2-[4-{[(2R)-2-Benzylpiperazin-1-yl]carbonyl}-5-(3-fluorophenyl)-1H-imidazol-1-yl]-1-(methoxymethyl)cyclohexanol

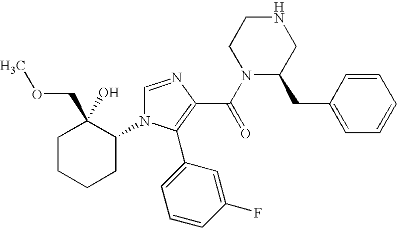

To tert-butyl (3R)-3-benzyl-4-({5-(3-fluorophenyl)-1-[cis-2-hydroxy-2-(methoxymethyl)cyclohexyl]-1H-imidazol-4-yl}carbonyl)piperazine-1-carboxylate (330 mg) was added TFA (3 ml), and the mixture was stirred at room temperature for 5 min, and poured into saturated aqueous sodium hydrogen carbonate. The mixture was extracted with ethyl acetate. The extract was washed successively with water and saturated brine, and dried over anhydrous magnesium sulfate, and the solvent was evaporated under reduced pressure. The residue was subjected to reversed-phase preparative HPLC (the purification conditions are described above), and the object fractions were collected, and partitioned between ethyl acetate and saturated aqueous sodium hydrogen carbonate. The organic layer was dried over anhydrous magnesium sulfate, and the solvent was evaporated under reduced pressure to give the object compound (103 mg).

MS (ESI+, m/e) 507 (M+1)

### Example 384

### (1R,2S)-2-[4-{[(2R)-2-Benzylpiperazin-1-yl]carbonyl}-5-(3-fluorophenyl)-1H-imidazol-1-yl]-1-(methoxymethyl)cyclohexanol

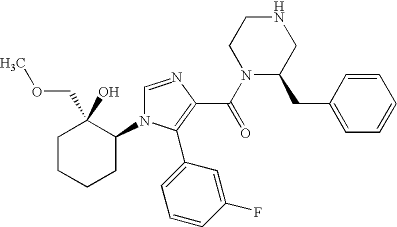

The fractions containing the other diastereomer obtained by the reversed-phase preparative HPLC in the above-mentioned Example 383 were collected, and partitioned between ethyl acetate and saturated aqueous sodium hydrogen carbonate. The organic layer was dried over anhydrous magnesium sulfate, and the solvent was evaporated under reduced pressure to give the object compound (109 mg).

MS (ESI+, m/e) 507 (M+1)

### Example 385

### 2-[4-({(2S)-2-[(Benzyloxy)methyl]piperazin-1-yl}carbonyl)-5-phenyl-1H-imidazol-1-yl]-1-(methoxymethyl)cyclohexanol and 2-(4-{[(2S)-2-(hydroxymethyl)piperazin-1-yl]carbonyl}-5-phenyl-1H-imidazol-1-yl)-1-(methoxymethyl)cyclohexanol

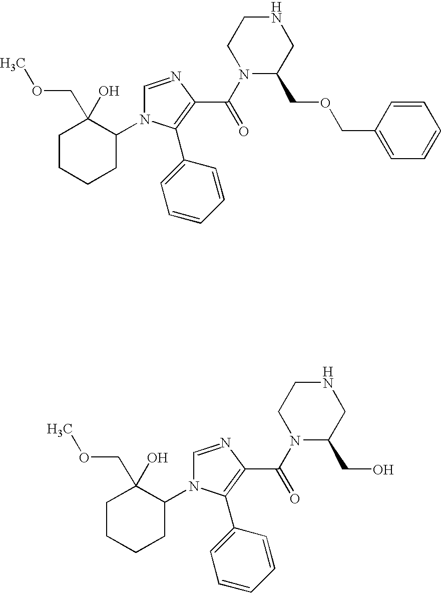

2-[4-({(2S)-4-Benzyl-2-[(benzyloxy)methyl]piperazin-1-yl}carbonyl)-5-phenyl-1H-imidazol-1-yl]-1-(methoxymethyl)cyclohexanol (530 mg) was dissolved in ethanol (15 ml), 20% palladium hydroxide-carbon (50% containing water, 100 mg) was added thereto, and the mixture was subjected to catalytic reduction at ambient temperature and normal pressure for 12 hr. The catalyst was filtered off, and the filtrate was concentrated under reduced pressure. The residue was subjected to reversed-phase preparative HPLC (the purification conditions are described above), and the object fractions were collected, and partitioned between ethyl acetate and saturated aqueous sodium hydrogen carbonate, respectively. The organic layers were dried over anhydrous magnesium sulfate, and the solvents were evaporated under reduced pressure to give 2-[4-({(2S)-2-[(benzyloxy)methyl]piperazin-1-yl}carbonyl)-5-phenyl-1H-imidazol-1-yl]-1-(methoxymethyl)cyclohexanol (244 mg) and 2-(4-{[(2S)-2-(hydroxymethyl)piperazin-1-yl]carbonyl}-5-phenyl-1H-imidazol-1-yl)-1-(methoxymethyl)cyclohexanol (9 mg).

MS (ESI+, m/e) 519 (M+1)

MS (ESI+, m/e) 429 (M+1)

### Example 386

### (1S,2R)-1-(Methoxymethyl)-2-(4-{[(2R)-2-(2-phenoxyethyl)piperazin-1-yl]carbonyl}-5-phenyl-1H-imidazol-1-yl)cyclohexanol

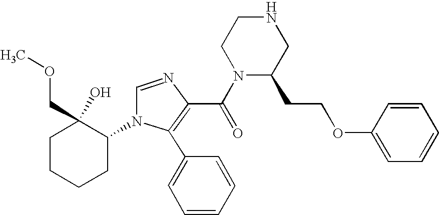

(1S,2R)-2-(4-{[(2R)-4-Benzyl-2-(2-phenoxyethyl)piperazin-1-yl]carbonyl}-5-phenyl-1H-imidazol-1-yl)-1-(methoxymethyl)cyclohexanol (32 mg) was dissolved in methanol (5 ml), 20% palladium hydroxide-carbon (50% containing water, 10 mg) was added thereto, and the mixture was subjected to catalytic reduction at ambient temperature and normal pressure for 12 hr. The catalyst was filtered off, and the filtrate was concentrated under reduced pressure to give the object compound (23 mg) as an amorphous solid.

MS (ESI+, m/e) 519 (M+1)

### Example 387

(the alternative synthetic method of the above-mentioned Example 386; The object compound was isolated as a dihydrochloride.)

### (1S,2R)-1-(Methoxymethyl)-2-(4-{[(2R)-2-(2-phenoxyethyl)piperazin-1-yl]carbonyl}-5-phenyl-1H-imidazol-1-yl)cyclohexanol dihydrochloride

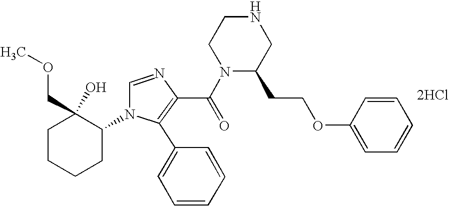

1-[(1R,2S)-2-Hydroxy-2-(methoxymethyl)cyclohexyl]-5-phenyl-1H-imidazole-4-carboxylic acid (330 mg) was suspended in DMF (10 ml), benzyl (3R)-3-(2-phenoxyethyl)piperazine-1-carboxylate hydrochloride (377 mg), WSC.HCl (288 mg), HOBt (184 mg) and triethylamine (0.279 ml) were added thereto, and the mixture was stirred at 60° C. for 5 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to silica gel column chromatography, and the fraction eluted with ethyl acetate was concentrated under reduced pressure to give benzyl (3R)-4-({1-[(1R,2S)-2-hydroxy-2-(methoxymethyl)cyclohexyl]-5-phenyl-1H-imidazol-4-yl}carbonyl)-3-(2-phenoxyethyl)piperazine-1-carboxylate (452 mg) as an amorphous solid. The total amount thereof was dissolved in methanol (50 ml), 20% palladium hydroxide-carbon (50% containing water, 50 mg) was added thereto, and the mixture was subjected to catalytic reduction at ambient temperature and normal pressure for 15 hr. The catalyst was filtered off, and the filtrate was concentrated under reduced pressure. The residue was dissolved in ethanol, the solution was acidified with 4N hydrogen chloride-ethyl acetate solution, and the solvent was evaporated under reduced pressure. Ethyl acetate was added to the residue, and the precipitated crystals were collected by filtration to give the object compound (334 mg).

MS (ESI+, m/e) 519 (M+1)

### Example 388

### (1S,2R)-1-(Methoxymethyl)-2-[5-phenyl-4-({(2R)-2-[2-(pyridin-3-yloxy)ethyl]piperazin-1-yl}carbonyl)-1H-imidazol-1-yl]cyclohexanol

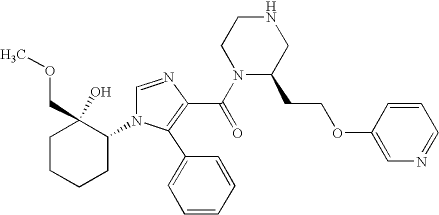

Benzyl (3R)-4-({1-[(1R,2S)-2-hydroxy-2-(methoxymethyl)cyclohexyl]-5-phenyl-1H-imidazol-4-yl}carbonyl)-3-{2-[(1-oxidopyridin-3-yl)oxy]ethyl}piperazine-1-carboxylate (80 mg) was dissolved in methanol (10 ml), 20% palladium hydroxide-carbon (50% containing water, 50 mg) was added thereto, and the mixture was subjected to catalytic reduction at ambient temperature and normal pressure for 12 hr. The catalyst was filtered off, and the filtrate was concentrated under reduced pressure. The residue was suspended in ethyl acetate, and the suspension was washed with saturated aqueous sodium hydrogen carbonate, and dried over anhydrous sodium sulfate, and the solvent was evaporated under reduced pressure to give the object compound (43 mg) as an amorphous solid.

MS (ESI+, m/e) 520 (M+1)

### Example 389

### (1S,2R)-2-[4-({(2R)-2-[2-(2-Fluorophenoxy)ethyl]piperazin-1-yl}carbonyl)-5-phenyl-1H-imidazol-1-yl]-1-(methoxymethyl)cyclohexanol

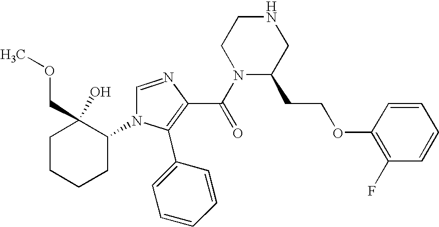

Benzyl (3R)-3-[2-(2-fluorophenoxy)ethyl]-4-({1-[(1R,2S)-2-hydroxy-2-(methoxymethyl)cyclohexyl]-5-phenyl-1H-imidazol-4-yl}carbonyl)piperazine-1-carboxylate (205 mg) was dissolved in methanol (2 ml), 20% palladium hydroxide-carbon (50% containing water, 200 mg) was added thereto, and the mixture was subjected to catalytic reduction at ambient temperature and normal pressure for 1 hr. The catalyst was filtered off, and the filtrate was concentrated under reduced pressure to give the object compound (145 mg) as an amorphous solid.

MS (ESI+, m/e) 537 (M+1)

### Example 390

### N-Cyclopropyl-4-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethoxy}benzamide

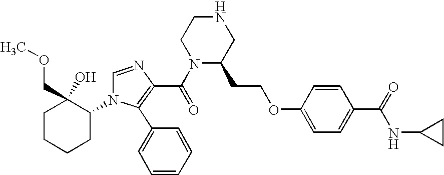

Benzyl (3R)-3-(2-{4-[(cyclopropylamino)carbonyl]phenoxy}ethyl)-4-({1-[(1R,2S)-2-hydroxy-2-(methoxymethyl)cyclohexyl]-5-phenyl-1H-imidazol-4-yl}carbonyl)piperazine-1-carboxylate (98 mg) was dissolved in methanol (10 ml), 20% palladium hydroxide-carbon (50% containing water, 20 mg) was added thereto, and the mixture was subjected to catalytic reduction at ambient temperature and normal pressure for 12 hr. The catalyst was filtered off, and the filtrate was concentrated under reduced pressure. The residue was suspended in ethyl acetate, and the suspension was washed with saturated aqueous sodium hydrogen carbonate, and dried over anhydrous sodium sulfate, and the solvent was evaporated under reduced pressure to give the object compound (60 mg) as an amorphous solid.

### Example 391

### (1S,2R)-2-[4-({(2R)-2-[2-(1H-Indazol-1-yl)ethyl]piperazin-1-yl}carbonyl)-5-phenyl-1H-imidazol-1-yl]-1-(methoxymethyl)cyclohexanol

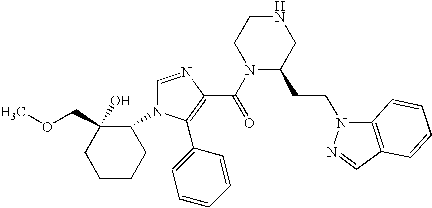

Benzyl (3R)-4-({1-[(1R,2S)-2-hydroxy-2-(methoxymethyl)cyclohexyl]-5-phenyl-1H-imidazol-4-yl}carbonyl)-3-[2-(1H-indazol-1-yl)ethyl]piperazine-1-carboxylate (140 mg) was dissolved in methanol (5 ml), 20% palladium hydroxide-carbon (50% containing water, 70 mg) was added thereto, and the mixture was subjected to catalytic reduction at ambient temperature and normal pressure for 12 hr. The catalyst was filtered off, and the filtrate was concentrated under reduced pressure to give the object compound (71 mg) as an amorphous solid.

MS (ESI+, m/e) 543 (M+1)

In the same manner as in Example 391, the following compound (Example 392) was obtained.

### Example 392

### (1S,2R)-2-[4-({(2R)-2-[2-(2H-Indazol-2-yl)ethyl]piperazin-1-yl}carbonyl)-5-phenyl-1H-imidazol-1-yl]-1-(methoxymethyl)cyclohexanol

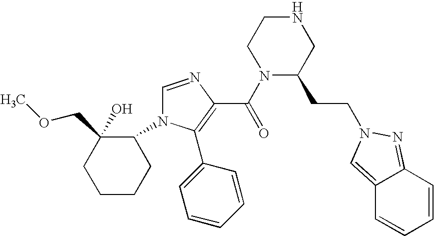

MS (ESI+, m/e) 543 (M+1)

### Example 393

### 1-Cyclopentyl-3-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethyl}-1,3-dihydro-2H-benzimidazol-2-one dihydrochloride

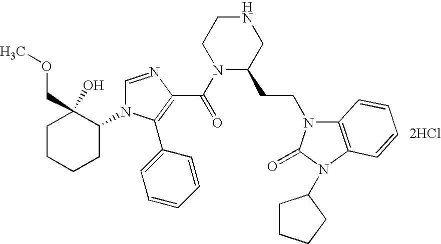

A solution of 1-[(1R,2S)-2-hydroxy-2-(methoxymethyl)cyclohexyl]-5-phenyl-1H-imidazole-4-carboxylic acid (150 mg), benzyl (3R)-3-{2-[3-(cyclopent-1-en-1-yl)-2-oxo-2,3-dihydro-1H-benzimidazol-1-yl]ethyl}piperazine-1-carboxylate hydrochloride (230 mg), WSC.HCl (180 mg), HOBt (70 mg) and triethylamine (220 μl) in DMF (7 ml) was stirred at 50° C. for 4 hr, and poured into water, and the mixture was extracted with ethyl acetate. The extract was washed with saturated brine, and dried over anhydrous magnesium sulfate, and the solvent was evaporated under reduced pressure. The residue was subjected to silica gel column chromatography, and the fraction eluted with ethyl acetate-hexane-methanol (1:1:0-9:0:1) was concentrated under reduced pressure to give benzyl (3R)-3-{2-[3-(cyclopent-1-en-1-yl)-2-oxo-2,3-dihydro-1H-benzimidazol-1-yl]ethyl}-4-({1-[(1R,2S)-2-hydroxy-2-(methoxymethyl)cyclohexyl]-5-phenyl-1H-imidazol-4-yl}carbonyl)piperazine-1-carboxylate (122 mg) as an amorphous solid. The total amount thereof was dissolved in methanol (4 ml), 20% palladium hydroxide-carbon (50% containing water, 20 mg) was added thereto, and the mixture was subjected to catalytic reduction at ambient temperature and normal pressure for 16 hr. The catalyst was filtered off, and the filtrate was concentrated under reduced pressure. The residue was subjected to reversed-phase preparative HPLC (the purification conditions are described above). The object fraction was diluted with saturated aqueous sodium hydrogen carbonate-saturated brine (1:1), and the mixture was extracted with chloroform. The extract was washed with saturated brine, and dried over anhydrous magnesium sulfate, and the solvent was evaporated under reduced pressure. The residue was treated with 2N hydrogen chloride-ethyl acetate solution to give the object compound (44 mg).

MS (ESI+, m/e) 627 (M+1)

### Example 394

### 1-Cyclohexyl-3-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethyl}-1,3-dihydro-2H-benzimidazol-2-one dihydrochloride

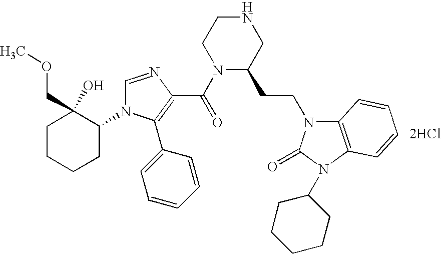

A solution of 1-[(1R,2S)-2-hydroxy-2-(methoxymethyl)cyclohexyl]-5-phenyl-1H-imidazole-4-carboxylic acid (150 mg), benzyl (3R)-3-{2-[3-(cyclohex-1-en-1-yl)-2-oxo-2,3-dihydro-1H-benzimidazol-1-yl]ethyl}piperazine-1-carboxylate hydrochloride (237 mg), WSC.HCl (180 mg), HOBt (70 mg) and triethylamine (220 μl) in DMF (7 ml) was stirred at 50° C. for 4 hr, and poured into water, and the mixture was extracted with ethyl acetate. The extract was washed with saturated brine, and dried over anhydrous magnesium sulfate, and the solvent was evaporated under reduced pressure. The residue was subjected to silica gel column chromatography, and the fraction eluted with ethyl acetate-hexane-methanol (1:1:0-9:0:1) was concentrated under reduced pressure to give benzyl (3R)-3-{2-[3-(cyclohex-1-en-1-yl)-2-oxo-2,3-dihydro-1H-benzimidazol-1-yl]ethyl}-4-({1-[(1R,2S)-2-hydroxy-2-(methoxymethyl)cyclohexyl]-5-phenyl-1H-imidazol-4-yl}carbonyl)piperazine-1-carboxylate (146 mg) as an amorphous solid. The total amount thereof was dissolved in methanol (4 ml), 20% palladium hydroxide-carbon (50% containing water, 20 mg) was added thereto, and the mixture was subjected to catalytic reduction at ambient temperature and normal pressure for 16 hr. The catalyst was filtered off, and the filtrate was concentrated under reduced pressure. The residue was subjected to reversed-phase preparative HPLC (the purification conditions are described above). The object fraction was diluted with saturated aqueous sodium hydrogen carbonate-saturated brine (1:1), and the mixture was extracted with chloroform. The extract was washed with saturated brine, and dried over anhydrous magnesium sulfate, and the solvent was evaporated under reduced pressure. The residue was treated with 2N hydrogen chloride-ethyl acetate solution to give the object compound (60 mg).

MS (ESI+, m/e) 641 (M+1)

### Example 395

### (1S,2R)-1-(Methoxymethyl)-2-[5-phenyl-4-({(2R)-2-[2-(1H-1,2,3-triazol-1-yl)ethyl]piperazin-1-yl}carbonyl)-1H-imidazol-1-yl]cyclohexanol

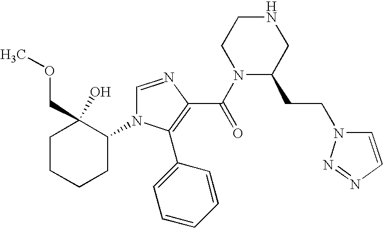

A solution of 1-[(1R,2S)-2-hydroxy-2-(methoxymethyl)cyclohexyl]-5-phenyl-1H-imidazole-4-carboxylic acid (409 mg), benzyl (3R)-3-[2-(4-acetyl-1H-1,2,3-triazol-1-yl)ethyl]piperazine-1-carboxylate hydrochloride (512 mg), WSC HCl (475 mg), HOBt (190 mg) and triethylamine (520 μl) in DMF (8 ml) was stirred at room temperature for 14 hr, and poured into saturated aqueous sodium hydrogen carbonate, and the mixture was extracted with ethyl acetate. The extract was washed with saturated brine, and dried over anhydrous magnesium sulfate, and the solvent was evaporated under reduced pressure. The residue was subjected to silica gel column chromatography, and the fraction eluted with ethyl acetate-hexane-methanol (1:9:0-17:0:3) was concentrated under reduced pressure to give benzyl (3R)-3-[2-(4-acetyl-1H-1,2,3-triazol-1-yl)ethyl]-4-({1-[(1R,2S)-2-hydroxy-2-(methoxymethyl)cyclohexyl]-5-phenyl-1H-imidazol-4-yl}carbonyl)piperazine-1-carboxylate (616 mg) as an amorphous solid. The total amount thereof was dissolved in ethanol (6 ml), 4N aqueous sodium hydroxide solution (2 ml) was added, and the mixture was stirred at 70° C. for 14 hr. The reaction mixture was poured into saturated aqueous sodium hydrogen carbonate, and the mixture was extracted with ethyl acetate. The extract was washed with saturated brine, and dried over anhydrous magnesium sulfate, and the solvent was evaporated under reduced pressure. The residue was subjected to basic silica gel column chromatography, and the fraction eluted with ethyl acetate-methanol (1:0-4:1) was concentrated under reduced pressure to give the object compound (16 mg).

MS (ESI+, m/e) 494 (M+1)

### Example 396

### (1R,2S)-2-[4-({2-[2-(2-Fluorophenyl)ethyl]piperazin-1-yl}carbonyl)-5-phenyl-1H-imidazol-1-yl]cyclohexanol

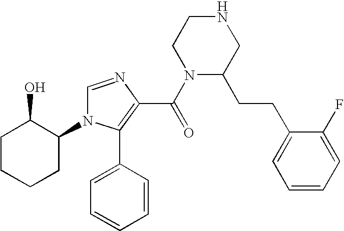

A solution of 1-[(1S,2R)-2-hydroxycyclohexyl]-5-phenyl-1H-imidazole-4-carboxylic acid (144 mg), (3R)-1-benzyl-3-[(E)-2-(2-fluorophenyl)vinyl]piperazine (158 mg), WSC.HCl (125 mg), HOBt (20 mg), N,N-diisopropylethylamine (181 μl) and DMAP (12 mg) in DMF (2 ml) was stirred at room temperature for 15 hr, and poured into saturated aqueous sodium hydrogen carbonate, and the mixture was extracted with ethyl acetate. The extract was washed successively with water and saturated brine, and dried over anhydrous magnesium sulfate, and the solvent was evaporated under reduced pressure. The residue was subjected to basic silica gel column chromatography, and the fraction eluted with ethyl acetate-hexane (1:1-1:0) was concentrated under reduced pressure to give (1R,2S)-2-[4-({(2R)-4-benzyl-2-[(E)-2-(2-fluorophenyl)vinyl]piperazin-1-yl}carbonyl)-5-phenyl-1H-imidazol-1-yl]cyclohexanol (184 mg) as an amorphous solid. The total amount thereof was dissolved in methanol (5 ml), 20% palladium hydroxide-carbon (50% containing water, 100 mg) was added thereto, and the mixture was subjected to catalytic reduction at ambient temperature and normal pressure for 15 hr. The catalyst was filtered off, and the filtrate was concentrated under reduced pressure. The crystals were collected by filtration to give the object compound (67 mg). (During the catalytic reduction, the racemization of the piperazine side chain proceeded together with the removal of the benzyl protecting group and the reduction of the unsaturated bond.)

MS (ESI+, m/e) 477 (M+1)

In the same manner as in Example 396, the following compounds (Examples 397-404) shown in Table 23 were obtained. (Each compound was isolated as a diastereomer mixture.)

TABLE 23

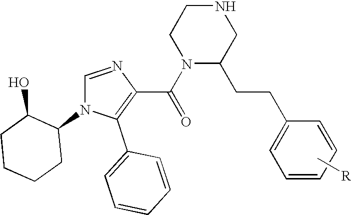

| Ex. No. | R | Compound | MS (ESI+) |
|---|---|---|---|
| 397 | 3-F | (1R,2S)-2-[4-({2-[2-(3- | 477 |
| | | Fluorophenyl)ethyl]piperazin-1- | |
| | | yl}carbonyl)-5-phenyl-1H-imidazol-1- | |
| | | yl]cyclohexanol | |
| 398 | 4-F | (1R,2S)-2-[4-({2-[2-(4- | 477 |
| | | Fluorophenyl)ethyl]piperazin-1- | |
| | | yl}carbonyl)-5-phenyl-1H-imidazol-1- | |
| | | yl]cyclohexanol | |
| 399 | 2-OCF₃ | (1R,2S)-2-{5-Phenyl-4-[(2-{2-[2- | 543 |
| | | (trifluoromethoxy)phenyl]ethyl} | |
| | | piperazin-1-yl)carbonyl]-1H-imidazol-1- | |
| | | yl}cyclohexanol | |
| 400 | 3-OCF₃ | (1R,2S)-2-{5-Phenyl-4-[(2-{2-[3- | 543 |
| | | (trifluoromethoxy)phenyl]ethyl} | |
| | | piperazin-1-yl)carbonyl]-1H-imidazol-1- | |
| | | yl}cyclohexanol | |
| 401 | 4-OCF₃ | (1R,2S)-2-{5-Phenyl-4-[(2-{2-[4- | 543 |
| | | (trifluoromethoxy)phenyl]ethyl} | |
| | | piperazin-1-yl)carbonyl]-1H-imidazol-1- | |
| | | yl}cyclohexanol | |
| 402 | 2-CF₃ | (1R,2S)-2-{5-Phenyl-4-[(2-{2-[2- | 527 |
| | | (trifluoromethoxy)phenyl]ethyl} | |
| | | piperazin-1-yl)carbonyl]-1H-imidazol-1- | |
| | | yl}cyclohexanol | |
| 403 | 3-CF₃ | (1R,2S)-2-{5-Phenyl-4-[(2-{2-[3- | 527 |
| | | (trifluoromethyl)phenyl]ethyl} | |
| | | piperazin-1-yl)carbonyl]-1H-imidazol-1- | |
| | | yl}cyclohexanol | |
| 404 | 4-CF₃ | (1R,2S)-2-{5-Phenyl-4-[(2-{2-[4- | 527 |
| | | (trifluoromethyl)phenyl]ethyl} | |
| | | piperazin-1-yl)carbonyl]-1H-imidazol-1- | |
| | | yl}cyclohexanol | |

In the same manner as in Example 396, the following compounds (Examples 405-412) shown in Table 24 were obtained. Each compound was isolated as a diastereomer by subjecting the diastereomer mixture to optical resolution by reversed-phase preparative HPLC (the purification conditions are described above). The final products were isolated as crystals or an amorphous solid in a free form or a hydrochloride by a known means such as phase transfer, liquid conversion, solvent extraction and the like. In the column of “Salt” in the Table, the compounds described as “-” were isolated as a free form.

TABLE 24

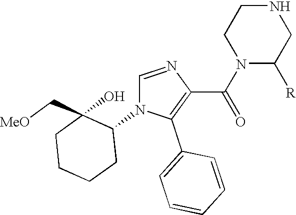

| Ex. | | | | |
|---|---|---|---|---|
| No. | R | salt | Compound | MS (ESI+) |
| 405 | 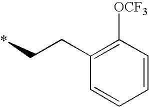 | — | (1S,2R)-1-(Methoxymethyl)-2-{5-phenyl- 4-[((2R)-2-{2-[2- (trifluoromethoxy)phenyl]ethyl} piperazin-1-yl)carbonyl]-1H-imidazol- 1-yl}cyclohexanol | 587 |
| 406 | 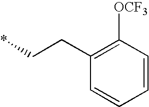 | — | (1S,2R)-1-(Methoxymethyl)-2-{5-phenyl- 4-[((2S)-2-{2-[2- (trifluoromethoxy)phenyl]ethyl} piperazin-1-yl)carbonyl]-1H-imidazol- 1-yl}cyclohexanol | 587 |
| 407 | 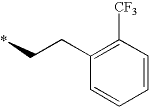 | — | (1S,2R)-1-(Methoxymethyl)-2-{5-phenyl- 4-[((2R)-2-{2-[2- (trifluoromethyl)phenyl]ethyl} piperazin-1-yl)carbonyl]-1H-imidazol- 1-yl}cyclohexanol | 571 |
| 408 | 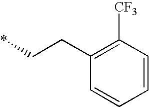 | — | (1S,2R)-1-(Methoxymethyl)-2-{5-phenyl- 4-[((2S)-2-{2-[2- (trifluoromethyl)phenyl]ethyl} piperazin-1-yl)carbonyl]-1H-imidazol- 1-yl}cyclohexanol | 571 |
| 409 | 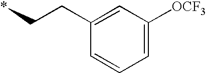 | HCl | (1S,2R)-1-(Methoxymethyl)-2-{5-phenyl- 4-[((2R)-2-{2-[3- (trifluoromethoxy)phenyl]ethyl} piperazin-1-yl)carbonyl]-1H-imidazol- 1-yl}cyclohexanol hydrochloride | 587 |
| 410 | 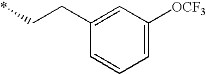 | HCl | (1S,2R)-1-(Methoxymethyl)-2-{5-phenyl- 4-[((2S)-2-{2-[3- (trifluoromethoxy)phenyl]ethyl} piperazin-1-yl)carbonyl]-1H-imidazol- 1-yl}cyclohexanol hydrochloride | 587 |
| 411 | 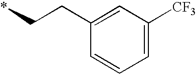 | HCl | (1S,2R)-1-(Methoxymethyl)-2-{5-phenyl- 4-[((2R)-2-{2-[3- (trifluoromethyl)phenyl]ethyl} piperazin-1-yl)carbonyl]-1H-imidazol- 1-yl}cyclohexanol hydrochloride | 571 |
| 412 | 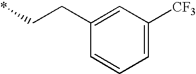 | HCl | (1S,2R)-1-(Methoxymethyl)-2-{5-phenyl- 4-[((2S)-2-{2-[3- (trifluoromethyl)phenyl]ethyl} piperazin-1-yl)carbonyl]-1H-imidazol- 1-yl}cyclohexanol hydrochloride | 571 |

### Example 413

### (1S,2R)-1-(Methoxymethyl)-2-(5-phenyl-4-{[2-(2-(piperidin-2-yl)ethyl)piperazin-1-yl]carbonyl}-1H-imidazol-1-yl)cyclohexanol

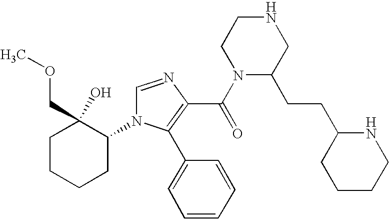

A mixture of 1-[(1R,2S)-2-hydroxy-2-(methoxymethyl)cyclohexyl]-5-phenyl-1H-imidazole-4-carboxylic acid (165 mg), (3R)-1-benzyl-3-[(E)-2-(pyridin-2-yl)vinyl]piperazine dihydrochloride (261 mg), WSC.HCl (192 mg), HOBt (306 mg), triethylamine (670 μl) and DMF (10 ml) was stirred at 60° C. for 5 hr, and poured into saturated aqueous sodium hydrogen carbonate, and the mixture was extracted with ethyl acetate. The extract was washed with saturated brine, and dried over anhydrous magnesium sulfate, and the solvent was evaporated under reduced pressure. The residue was subjected to basic silica gel column chromatography, and the fraction eluted with ethyl acetate-hexane-methanol (1:1:0-7:0:3) was concentrated under reduced pressure to give (1S,2R)-2-[4-({4-benzyl-2-[(E)-2-(pyridin-2-yl)vinyl]piperazin-1-yl}carbonyl)-5-phenyl-1H-imidazol-1-yl]-1-(methoxymethyl)cyclohexanol (208 mg) as an amorphous solid. The total amount thereof was dissolved in methanol (6 ml), 20% palladium hydroxide-carbon (50% containing water, 50 mg) was added thereto, and the mixture was subjected to catalytic reduction at ambient temperature and normal pressure for 15 hr. The catalyst was filtered off, and the filtrate was concentrated under reduced pressure to give the object compound (120 mg). (During the catalytic reduction, the racemization of the piperazine side chain and the reduction of the pyridine ring proceeded together with the removal of the benzyl protecting group and the reduction of the unsaturated bond.)

MS (ESI+, m/e) 510 (M+1)

### Example 414

### (1R,2S)-2-{4-[(2-Pentylpiperazin-1-yl)carbonyl]-5-phenyl-1H-imidazol-1-yl}cyclohexanol

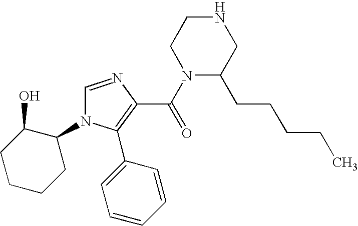

(1R,2S)-2-[4-({(2R)-4-Benzyl-2-[(E)-2-cyclopropylvinyl]piperazin-1-yl}carbonyl)-5-phenyl-1H-imidazol-1-yl]cyclohexanol (100 mg) was dissolved in methanol (3 ml), 20% palladium hydroxide-carbon (50% containing water, 30 mg) was added thereto, and the mixture was subjected to catalytic reduction at ambient temperature and normal pressure for 12 hr. The catalyst was filtered off, and the filtrate was concentrated under reduced pressure. The residue was suspended in ethyl acetate, the suspension was dried over anhydrous sodium sulfate, and the solvent was evaporated under reduced pressure. The residue was subjected to silica gel column chromatography, and the fraction eluted with ethyl acetate-methanol (1:1) was concentrated under reduced pressure. The residue was vacuum-dried to give the object compound (25 mg) as an amorphous solid. (During the catalytic reduction, the ring-opening of the cyclopropyl group and the racemization of the piperazine side chain proceeded together with the removal of the benzyl protecting group and the reduction of the unsaturated bond.)

MS (ESI+, m/e) 425 (M+1)

### Example 415

### (1S,2R)-2-{4-[((2R)-2-{2-Hydroxy-2-[6-(trifluoromethyl)piperidin-2-yl]ethyl}piperazin-1-yl)carbonyl]-5-phenyl-1H-imidazol-1-yl}-1-(methoxymethyl)cyclohexanol trihydrochloride

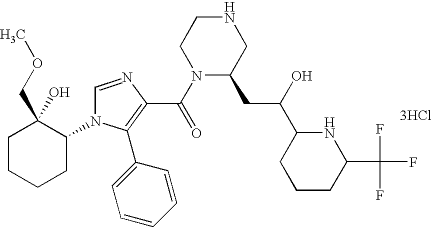

(1S,2R)-2-{4-[((2R)-2-{(2RS)-2-Hydroxy-2-[6-(trifluoromethyl)pyridin-2-yl]ethyl}piperazin-1-yl)carbonyl]-5-phenyl-1H-imidazol-1-yl}-1-(methoxymethyl)cyclohexanol trihydrochloride (1:1 mixture of the compounds of Example 199 and 200, 104 mg) was dissolved in methanol (10 ml), 20% palladium hydroxide-carbon (50% containing water, 50 mg) was added thereto, and the mixture was subjected to catalytic reduction at ambient temperature and normal pressure for 12 hr. The catalyst was filtered off, and the filtrate was concentrated under reduced pressure. The residue was treated with 4N hydrogen chloride-ethyl acetate solution to give the object compound (103 mg). (The hydroxyl group was not removed, and the reduction of the pyridine ring alone proceeded.)

MS (ESI+, m/e) 593 (M+1)

### Example 416

### 4-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}benzoic acid trifluoroacetate

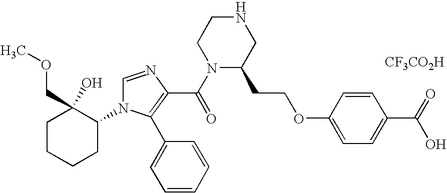

Benzyl (3R)-4-({1-[(1R,2S)-2-hydroxy-2-(methoxymethyl)cyclohexyl]-5-phenyl-1H-imidazol-4-yl}carbonyl)-3-{2-[4-(methoxycarbonyl)phenoxy]ethyl}piperazine-1-carboxylate (486 mg) was dissolved in ethanol (8 ml), 4N aqueous sodium hydroxide solution (4 ml) was added, and the mixture was stirred at 60° C. for 15 hr. The reaction mixture was concentrated under reduced pressure, the residue was subjected to reversed-phase preparative HPLC (the purification conditions are described above), and the object fraction was concentrated under reduced pressure to give the object compound (237 mg).

MS (ESI+, m/e) 563 (M+1)

In the same manner as in Example 416, the following compounds (Examples 417-418) were obtained.

### Example 417

### 3-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}benzoic acid trifluoroacetate

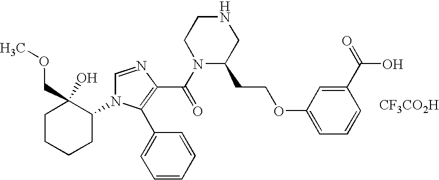

MS (ESI+, m/e) 563 (M+1)

### Example 418

### 2-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}benzoic acid bistrifluoroacetate

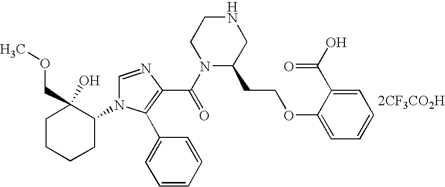

MS (ESI+, m/e) 563 (M+1)

In the same manner as in Example 416 except that the final product was isolated as a dihydrochloride by a known operation such as phase transfer, liquid conversion, solvent extraction and the like, the following compound (Example 419) was obtained.

### Example 419

### (1S,2R)-1-(Methoxymethyl)-2-{4-[((2R)-2-{2-[(1-oxidopyridin-3-yl)oxy]ethyl}piperazin-1-yl)carbonyl]-5-phenyl-1H-imidazol-1-yl}cyclohexanol dihydrochloride

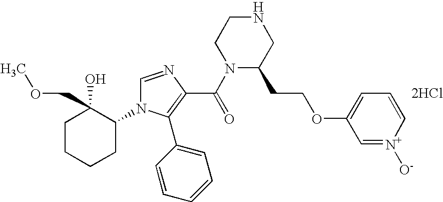

MS (ESI+, m/e) 536 (M+1)

### Example 420

### 6-{[(2S)-1-({1-[(1R,2S)-2-Hydroxy-2-(methoxymethyl)cyclohexyl]-5-phenyl-1H-imidazol-4-yl}carbonyl)piperazin-2-yl]methoxy}nicotinic acid

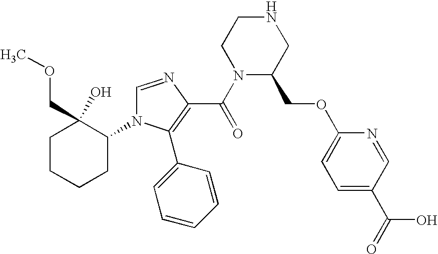

A mixture of methyl 6-{[(2S)-1-({1-[(1R,2S)-2-hydroxy-2-(methoxymethyl)cyclohexyl]-5-phenyl-1H-imidazol-4-yl}carbonyl)piperazin-2-yl]methoxy}nicotinate trihydrochloride (the compound of Example 198, 220 mg), lithium hydroxide monohydrate (140 mg), methanol (3 ml) and water (3 ml) was stirred at room temperature for 3 days, and methanol was evaporated under reduced pressure. The residual aqueous solution was adjusted with 1N hydrochloric acid to pH 6-8. The solution was subjected to DIAION HP-20 (manufactured by Mitsubishi Chemical), and washed with water. The fraction eluted with acetone was concentrated under reduced pressure to about ⅓ volume, and the resulting crystals were collected by filtration to give the object compound (147 mg).

MS (ESI+, m/e) 550 (M+1)

### Example 421

### (4-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}phenyl)acetic acid

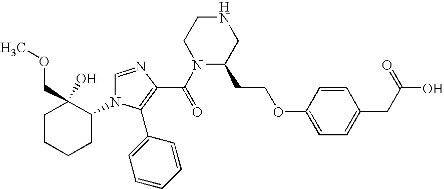

Methyl (4-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethoxy}phenyl)acetate (the compound of Example 261) (125 mg) was dissolved in methanol (3 ml), potassium hydroxide (36 mg) was added, and the mixture was stirred at 65° C. for 15 hr. The reaction mixture was concentrated under reduced pressure, and the residue was neutralized with 1N hydrochloric acid. The mixture was again concentrated under reduced pressure, and the residue was extracted with chloroform. The extract was washed with saturated brine, and dried over anhydrous sodium sulfate, and the solvent was evaporated under reduced pressure to give the object compound (62 mg) as an amorphous solid.

MS (ESI+, m/e) 577 (M+1)

### Example 422

### (1S,2R)-1-(Methoxymethyl)-2-[5-phenyl-4-({(2R)-2-[2-(4-(piperazin-1-yl)phenoxy)ethyl]piperazin-1-yl}carbonyl)-1H-imidazol-1-yl]cyclohexanol

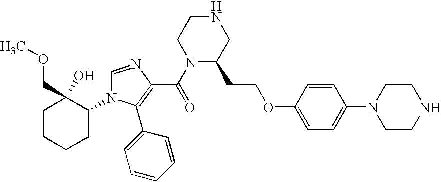

Benzyl (3R)-3-{2-[4-(4-acetylpiperazin-1-yl)phenoxy]ethyl}-4-({1-[(1R,2S)-2-hydroxy-2-(methoxymethyl)cyclohexyl]-5-phenyl-1H-imidazol-4-yl}carbonyl)piperazine-1-carboxylate (120 mg) was dissolved in ethanol (2 ml), 4N aqueous sodium hydroxide solution (2 ml) was added, and the mixture was stirred 65° C. for 5 hr. The reaction mixture was concentrated under reduced pressure, and water was added to the residue, and the liberated oil was extracted with ethyl acetate. The extract was washed with saturated brine, and dried over anhydrous sodium sulfate, and the solvent was evaporated under reduced pressure. The residue was subjected to basic silica gel column chromatography, and the fraction eluted with ethyl acetate-hexane-methanol (1:1:0-10:0:1) was concentrated under reduced pressure to give the object compound (80 mg) as an amorphous solid.

MS (ESI+, m/e) 603 (M+1)

### Example 423

### 4-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}-3-methoxybenzamide dihydrochloride

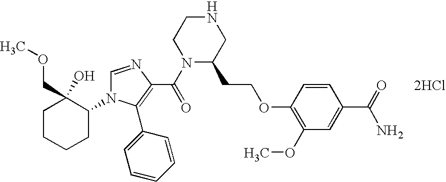

Benzyl (3R)-3-[2-(4-cyano-2-methoxyphenoxy)ethyl]-4-({1-[(1R,2S)-2-hydroxy-2-(methoxymethyl)cyclohexyl]-5-phenyl-1H-imidazol-4-yl}carbonyl)piperazine-1-carboxylate (25 mg) was dissolved in ethanol (2 ml), 4N aqueous sodium hydroxide solution (2 ml) was added thereto, and the mixture was stirred at 65° C. for 5 hr. The reaction mixture was concentrated under reduced pressure, water was added to the residue, and the liberated oil was extracted with ethyl acetate. The extract was washed with saturated brine, and dried over anhydrous sodium sulfate, and the solvent was evaporated under reduced pressure. The residue was subjected to reversed-phase preparative HPLC (the purification conditions are described above). The object fraction was diluted with saturated aqueous sodium hydrogen carbonate, and the mixture was extracted with ethyl acetate. The extract was dried over anhydrous magnesium sulfate, and concentrated under reduced pressure. The residue was treated with 4N hydrogen chloride-ethyl acetate solution to give the object compound (3 mg).

MS (ESI+, m/e) 592 (M+1)

### Example 424

### (1S,2R)-2-{4-[((2R)-2-{2-[2-(1-Hydroxyethyl)phenoxy]ethyl}piperazin-1-yl)carbonyl]-5-phenyl-1H-imidazol-1-yl}-1-(methoxymethyl)cyclohexanol

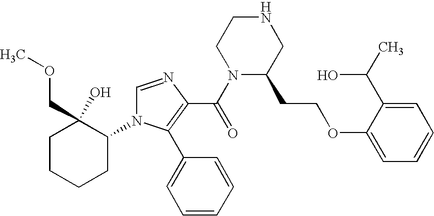

1-(2-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}phenyl)ethanone (the compound of Example 240, 105 mg) was dissolved in methanol (5 ml), and the solution was ice-cooled. Sodium borohydride (11 mg) was added, and the mixture was stirred at 0° C. for 5 hr. To the reaction mixture was added saturated aqueous sodium hydrogen carbonate, and the mixture was extracted with ethyl acetate. The extract was washed with saturated brine, dried over anhydrous magnesium sulfate, and concentrated under reduced pressure. The residue was subjected to reversed-phase preparative HPLC (the purification conditions are described above). The object fraction was neutralized with saturated aqueous sodium hydrogen carbonate, and the mixture was extracted with ethyl acetate. The extract was dried over anhydrous sodium sulfate, and the solvent was evaporated under reduced pressure to give the object compound (63 mg).

MS (ESI+, m/e) 563 (M+1)

### Example 425

### (1S,2R)-2-{4-[((2R)-2-{2-[3-(1-Hydroxyethyl)phenoxy]ethyl}piperazin-1-yl)carbonyl]-5-phenyl-1H-imidazol-1-yl}-1-(methoxymethyl)cyclohexanol

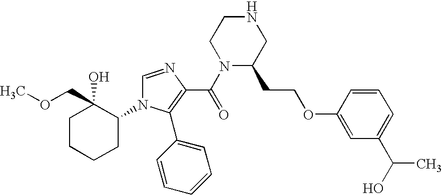

1-(3-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}phenyl)ethanone (the compound of Example 233) (50 mg) was dissolved in methanol (10 ml), and the solution was ice-cooled. Sodium borohydride (4 mg) was added, and the mixture was stirred at room temperature for 1 hr. The reaction mixture was poured into saturated aqueous sodium hydrogen carbonate, and the mixture was extracted with ethyl acetate. The extract was washed with saturated brine, and dried over anhydrous magnesium sulfate, and the solvent was evaporated under reduced pressure. The residue was subjected to silica gel column chromatography, and the object fraction was concentrated under reduced pressure to give the object compound (14 mg) as an amorphous solid.

MS (ESI+, m/e) 563 (M+1)

### Example 426

### (1S,2R)-2-{4-[((2R)-2-{2-[4-(1-Hydroxyethyl)phenoxy]ethyl}piperazin-1-yl)carbonyl]-5-phenyl-1H-imidazol-1-yl}-1-(methoxymethyl)cyclohexanol

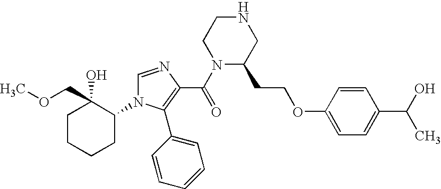

1-(4-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}phenyl)ethanone (the compound of Example 231) (50 mg) was dissolved in methanol (10 ml), and the solution was ice-cooled. Sodium borohydride (4 mg) was added, and the mixture was stirred at room temperature for 1 hr. The reaction mixture was poured into saturated aqueous sodium hydrogen carbonate, and the mixture was extracted with ethyl acetate. The extract was washed with saturated brine, and dried over anhydrous magnesium sulfate, and the solvent was evaporated under reduced pressure. The residue was subjected to silica gel column chromatography, and the object fraction was concentrated under reduced pressure to give the object compound (13 mg) as an amorphous solid.

MS (ESI+, m/e) 563 (M+1)

In the same manner as in Example 3 (Method C), the following compound (Example 427) was obtained.

### Example 427

### (1S,2R)-2-(4-{[(2R)-2-Benzyl-2-methylpiperazin-1-yl]carbonyl}-5-phenyl-1H-imidazol-1-yl)-1-(methoxymethyl)cyclohexanol dihydrochloride

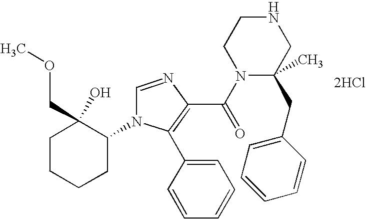

MS (ESI+, m/e) 503 (M+1)

### Example 428

### (1S,2R)-2-(4-{[(2R)-2-(2-Anilinoethyl)piperazin-1-yl]carbonyl}-5-phenyl-1H-imidazol-1-yl)-1-(methoxymethyl)cyclohexanol

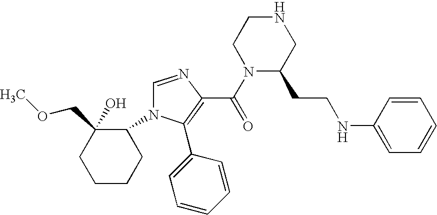

A solution of 1-[(1R,2S)-2-hydroxy-2-(methoxymethyl)cyclohexyl]-5-phenyl-1H-imidazole-4-carboxylic acid (650 mg), benzyl (3R)-3-(2-anilinoethyl)piperazine-1-carboxylate (800 mg), WSC.HCl (566 mg) and HOBt (360 mg) in DMF (10 ml) was stirred at room temperature for 15 hr, and poured into saturated aqueous sodium hydrogen carbonate, and the mixture was extracted with ethyl acetate. The extract was washed successively with water and saturated brine, and dried over anhydrous magnesium sulfate, and the solvent was evaporated under reduced pressure. The residue was subjected to silica gel column chromatography, and the fraction eluted with ethyl acetate-methanol (1:0-9:1) was concentrated under reduced pressure to give benzyl (3R)-3-(2-anilinoethyl)-4-({1-[(1R,2S)-2-hydroxy-2-(methoxymethyl)cyclohexyl]-5-phenyl-1H-imidazol-4-yl}carbonyl)piperazine-1-carboxylate (850 mg) as an amorphous solid. 120 mg therefrom was dissolved in methanol (2 ml), 4N aqueous sodium hydroxide solution (2 ml) was added thereto, and the mixture was stirred at 60° C. for 8 hr. The reaction mixture was concentrated under reduced pressure, and the residue was suspended in water, and the suspension was extracted with ethyl acetate. The extract was dried over anhydrous sodium sulfate, and the solvent was evaporated under reduced pressure. The residue was subjected to basic silica gel column chromatography, and the fraction eluted with ethyl acetate-methanol (1:0-9:2) was concentrated under reduced pressure to give the object compound (50 mg) as an amorphous solid.

MS (ESI+, m/e) 518 (M+1)

### Example 429

### (1S,2R)-1-(Methoxymethyl)-2-{4-[((2R)-2-{2-[methyl(phenyl)amino]ethyl}piperazin-1-yl)carbonyl]-5-phenyl-1H-imidazol-1-yl}cyclohexanol trihydrochloride

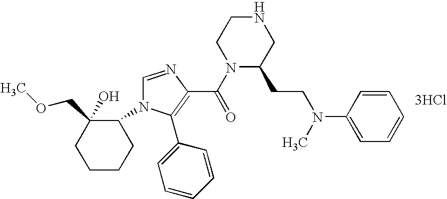

A solution of 1-[(1R,2S)-2-hydroxy-2-(methoxymethyl)cyclohexyl]-5-phenyl-1H-imidazole-4-carboxylic acid (165 mg), benzyl (3R)-3-{2-[methyl(phenyl)amino]ethyl}piperazine-1-carboxylate (195 mg), WSC.HCl (144 mg) and HOBt (92 mg) in DMF (10 ml) was stirred at room temperature for 15 hr, and poured into saturated aqueous sodium hydrogen carbonate, and the mixture was extracted with ethyl acetate. The extract was washed successively with water and saturated brine, and dried over anhydrous magnesium sulfate, and the solvent was evaporated under reduced pressure. The residue was subjected to silica gel column chromatography, and the fraction eluted with ethyl acetate-methanol (1:0-9:1) was concentrated under reduced pressure to give benzyl (3R)-4-({1-[(1R,2S)-2-hydroxy-2-(methoxymethyl)cyclohexyl]-5-phenyl-1H-imidazol-4-yl}carbonyl)-3-{2-[methyl(phenyl)amino]ethyl}piperazine-1-carboxylate (180 mg) as an amorphous solid. 160 mg therefrom was dissolved in methanol (5 ml), 20% palladium hydroxide-carbon (50% containing water, 80 mg) was added thereto, and the mixture was subjected to catalytic reduction at ambient temperature and normal pressure for 1 hr. The catalyst was filtered off, and the filtrate was concentrated under reduced pressure. The residue was subjected to basic silica gel column chromatography, and the fraction eluted with ethyl acetate-methanol (1:0-9:2) was concentrated under reduced pressure. The residue was dissolved in ethyl acetate, and the solution was acidified with 4N hydrogen chloride-ethyl acetate solution, and concentrated under reduced pressure to give the object compound (130 mg) as an amorphous solid.

MS (ESI+, m/e) 532 (M+1)

In the same manner as in the above-mentioned Example 1 (Method A)-Example 15 (Method O), the following compounds (Examples 430-567) shown in Table 25-1-Table 25-2, Table 26, Table 27-1-Table 27-2 and Table 28-1-Table 28-8 were obtained. Where necessary, each compound was isolated and purified by a known means such as phase transfer, liquid conversion, solvent extraction, silica gel column chromatography, reversed-phase preparative HPLC and the like. The final products were isolated as a hydrochloride by a treatment with 4N hydrogen chloride-ethyl acetate solution, as in Method A and the like, or isolated as crystals or an amorphous solid in a free from, as in Method B and the like. In the column of “Salt” in the Tables, the compounds described as “-” were isolated as a free form.

TABLE 25-1

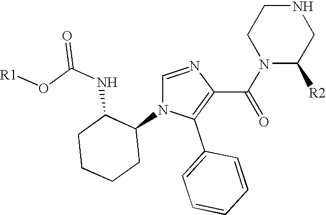

| Ex. No. | R1 | R2 | Method | Salt | MS (ESI+) |
|---|---|---|---|---|---|
| 430 | Me | 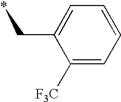 | A | — | 570 |
| 431 | Me | 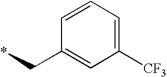 | A | — | 570 |
| 432 | Me | 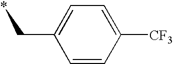 | A | — | 570 |
| 433 | Me | 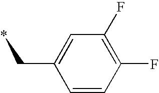 | A | — | 538 |
| 434 | Me | 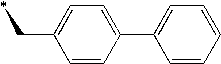 | A | — | 578 |
| 435 | Me | 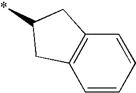 | A | — | 528 |
| 436 | Et | 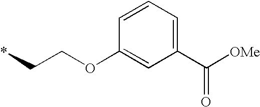 | L | — | 604 |
| 437 | Me | 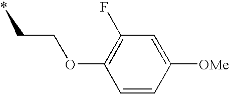 | L | — | 580 |
| 438 | Et | 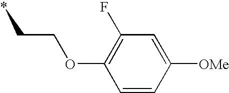 | L | — | 594 |
| 439 | Me | 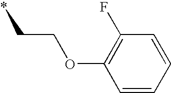 | L | — | 550 |
| 440 | Et | 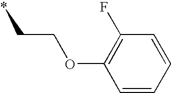 | L | — | 564 |
| 441 | Me | 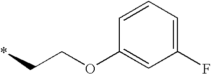 | L | — | 550 |

TABLE 25-2

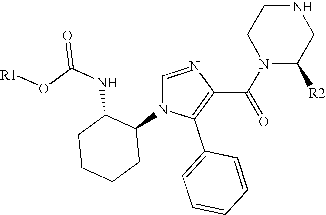

| Ex. No. | R1 | R2 | Method | Salt | MS (ESI+) |
|---|---|---|---|---|---|
| 442 | Et | 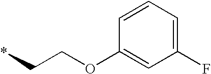 | L | — | 564 |
| 443 | Me | 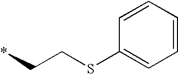 | M | — | 548 |
| 444 | Me | 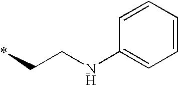 | A | — | 531 |
| 445 | Me | 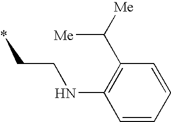 | A | — | 573 |
| 446 | Me | 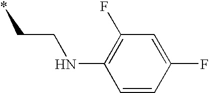 | A | — | 567 |
| 447 | Me | 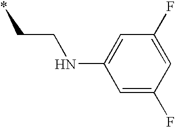 | A | — | 567 |
| 448 | Et | 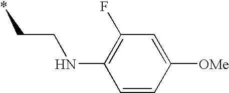 | A | — | 593 |
| 449 | Et | 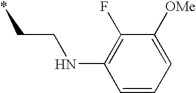 | I | — | 593 |
| 450 | Me | 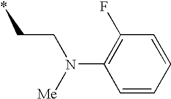 | L | — | 563 |

TABLE 26

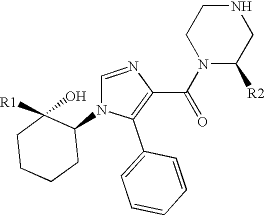

| Ex. No. | R1 | R2 | Method | Salt | MS (ESI+) |
|---|---|---|---|---|---|
| 451 | 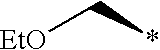 | 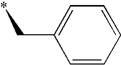 | C | 2HCl | 503 |
| 452 | Me | 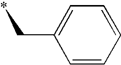 | C | 2HCl | 459 |
| 453 | Et | 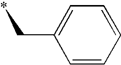 | C | 2HCl | 473 |
| 454 | 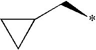 | 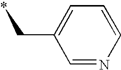 | A | — | 500 |
| 455 | 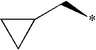 | 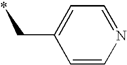 | A | — | 500 |
| 456 | Et | 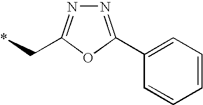 | A | 2HCl | 541 |
| 457 | 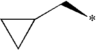 | 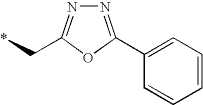 | A | — | 567 |
| 458 | 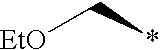 | 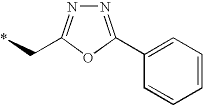 | A | — | 571 |
| 459 | 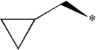 | 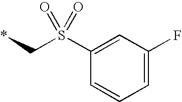 | I | — | 581 |
| 460 | Me | 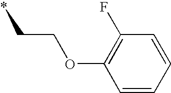 | M | — | 507 |
| 461 | Me | 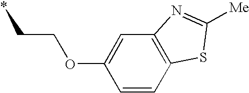 | J | — | 560 |
| 462 | Me | 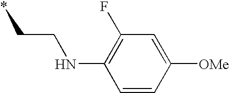 | A | — | 536 |
| 463 | Me | 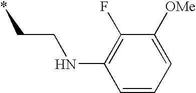 | I | — | 536 |

TABLE 27-1

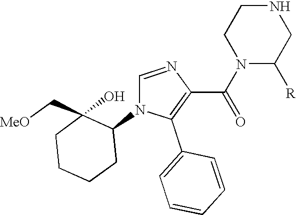

| | | | | MS |
|---|---|---|---|---|
| Ex. No. | R | Method | Salt | (ESI+) |
| 464 | 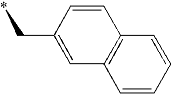 | A | HCl | 539 |
| 465 | 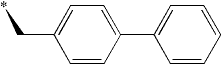 | A | HCl | 565 |
| 466 | 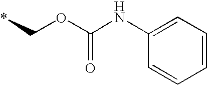 | F | — | 548 |
| 467 | 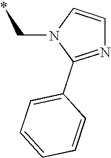 | C | 3HCl | 555 |
| 468 | 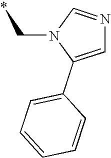 | C | 3HCl | 555 |
| 469 | 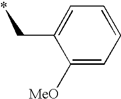 | F | — | 519 |
| 470 | 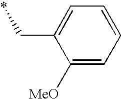 | F | — | 519 |
| 471 | 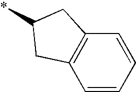 | A | — | 515 |
| 472 | 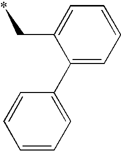 | F | — | 565 |
| 473 | 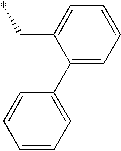 | F | — | 565 |

TABLE 27-2

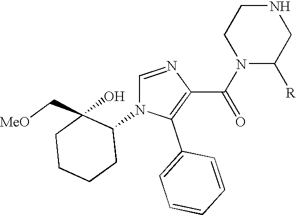

| Ex. | | | | MS |
|---|---|---|---|---|
| No. | R | Method | Salt | (ESI+) |
| 474 | 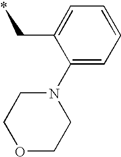 | F | — | 574 |
| 475 | 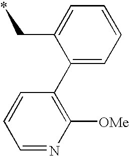 | F | — | 596 |
| 476 | 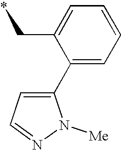 | F | — | 569 |
| 477 | 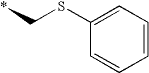 | H | 2HCl | 521 |
| 478 | 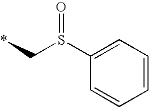 | G | — | 537 |
| 479 | 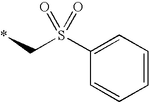 | H | 2HCl | 553 |
| 480 | 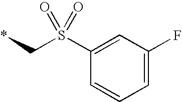 | I | 2HCl | 571 |
| 481 | 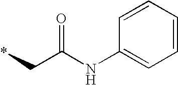 | A | — | 532 |
| 482 | 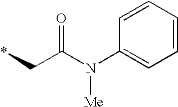 | A | — | 546 |
| 483 | 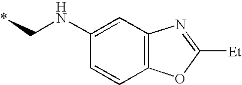 | J | — | 560 |

TABLE 28-1

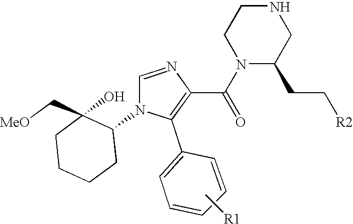

| Ex. No. | R1 | R2 | Method | Salt | MS (ESI+) |
|---|---|---|---|---|---|
| 484 | H | 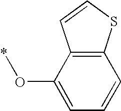 | J | — | 575 |
| 485 | H | 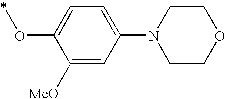 | I | 2HCl | 634 |
| 486 | H | 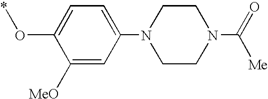 | I | 2HCl | 675 |
| 487 | H | 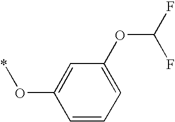 | I | HCl | 585 |
| 488 | H | 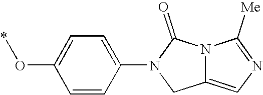 | L | — | 654 |
| 489 | H | 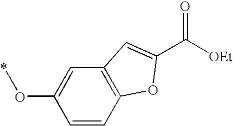 | L | — | 631 |
| 490 | H | 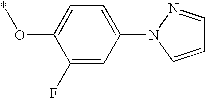 | H | 2HCl | 603 |
| 491 | H | 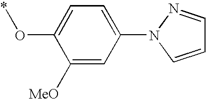 | L | — | 560 |
| 492 | 3-F | 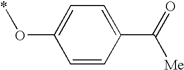 | I | 2HCl | 579 |
| 493 | H | 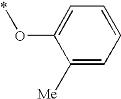 | L | — | 533 |
| 494 | H | 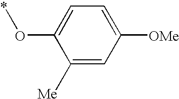 | L | — | 563 |

TABLE 28-2

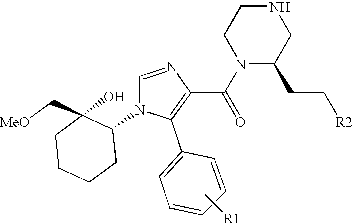

| | | | | | MS |
|---|---|---|---|---|---|
| Ex. No. | R1 | R2 | Method | Salt | (ESI+) |
| 495 | H | 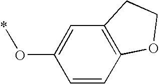 | L | — | 561 |
| 496 | H | 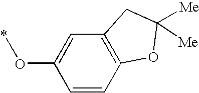 | I | — | 589 |
| 497 | H | 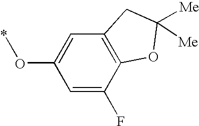 | I | — | 607 |
| 498 | H | 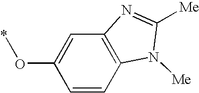 | L | — | 587 |
| 499 | H | 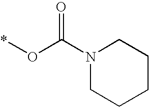 | H | 2HCl | 554 |
| 500 | H | 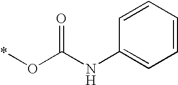 | H | 2HCl | 562 |
| 501 | H | 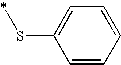 | M | 2HCl | 535 |
| 502 | H | 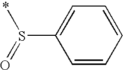 | M | — | 551 |
| 503 | H | 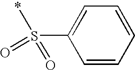 | M | 2HCl | 567 |
| 504 | H | 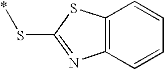 | M | — | 592 |
| 505 | H | 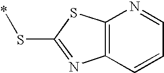 | J | — | 593 |
| 506 | H | 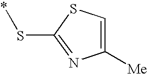 | M | — | 556 |

TABLE 28-3

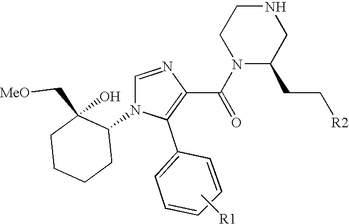

| | | | | | MS |
|---|---|---|---|---|---|
| Ex. No. | R1 | R2 | Method | Salt | (ESI+) |
| 507 | H | 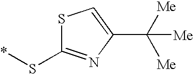 | J | — | 598 |
| 508 | H | 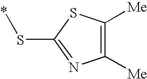 | J | — | 570 |
| 509 | H | 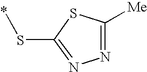 | M | — | 557 |
| 510 | H | 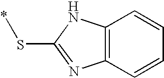 | M | — | 575 |
| 511 | H | 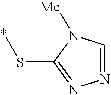 | M | — | 540 |
| 512 | H | 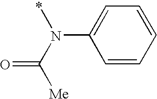 | O | — | 560 |
| 513 | H | 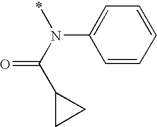 | O | — | 586 |
| 514 | H | 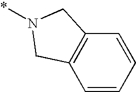 | H | 3HCl | 544 |
| 515 | H | 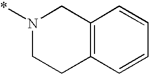 | H | 3HCl | 558 |
| 516 | H | 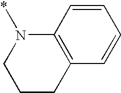 | H | 3HCl | 558 |
| 517 | H | 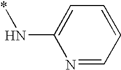 | H | — | 519 |
| 518 | H | 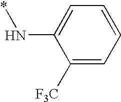 | I | — | 586 |

TABLE 28-4

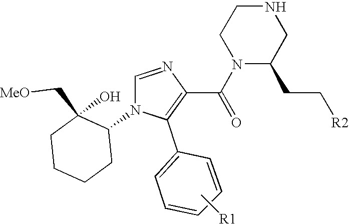

| Ex. | | | | | |
|---|---|---|---|---|---|
| No. | R1 | R2 | Method | Salt | (ESI+) |
| 519 | H | 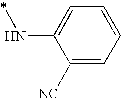 | I | — | 543 |
| 520 | H | 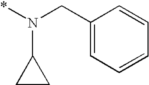 | K | — | 572 |
| 521 | H | 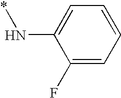 | I | — | 536 |
| 522 | H | 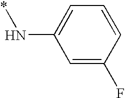 | A | — | 536 |
| 523 | H | 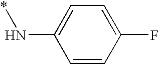 | A | — | 536 |
| 524 | H | 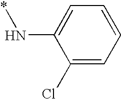 | K | — | 552 |
| 525 | H | 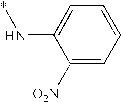 | K | — | 563 |
| 526 | H | 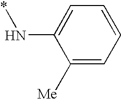 | A | — | 532 |
| 527 | H | 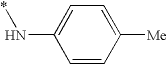 | A | — | 532 |
| 528 | H | 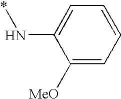 | O | — | 548 |
| 529 | H | 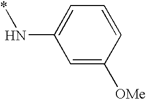 | O | — | 548 |
| 530 | H | 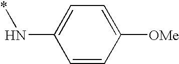 | I | — | 548 |

TABLE 28-5

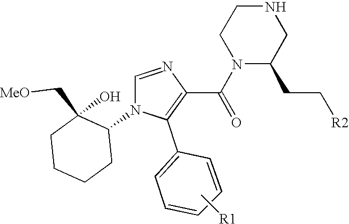

| Ex. | | | | | MS |
|---|---|---|---|---|---|
| No. | R1 | R2 | Method | Salt | (ESI+) |
| 531 | H | 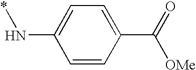 | I | — | 576 |
| 532 | H | 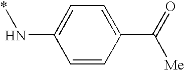 | K | — | 560 |
| 533 | H | 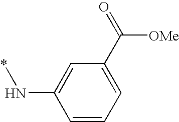 | O | — | 576 |
| 534 | H | 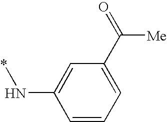 | K | — | 560 |
| 535 | H | 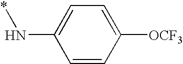 | I | — | 602 |
| 536 | H | 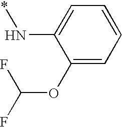 | A | — | 584 |
| 537 | H | 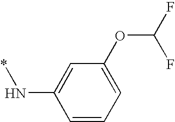 | A | — | 584 |
| 538 | H | 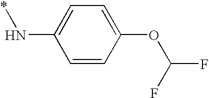 | A | — | 584 |
| 539 | H | 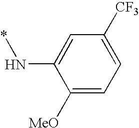 | A | — | 616 |
| 540 | H | 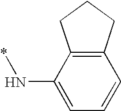 | A | — | 558 |
| 541 | H | 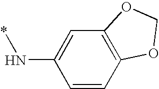 | A | — | 562 |

TABLE 28-6

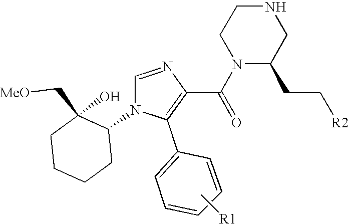

| Ex. | | | | | MS |
|---|---|---|---|---|---|
| No. | R1 | R2 | Method | Salt | (ESI+) |
| 542 | H | 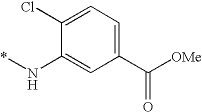 | J | — | 610 |
| 543 | H | 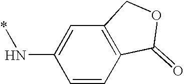 | I | — | 574 |
| 544 | H | 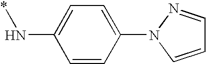 | I | — | 584 |
| 545 | H | 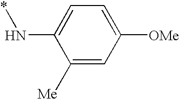 | A | — | 562 |
| 546 | H | 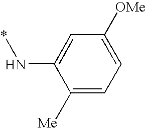 | A | — | 562 |
| 547 | H | 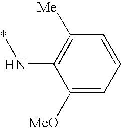 | A | — | 562 |
| 548 | H | 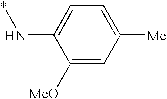 | A | — | 562 |
| 549 | H | 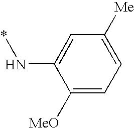 | A | — | 562 |
| 550 | H | 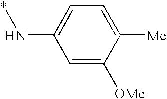 | A | — | 562 |
| 551 | H | 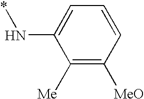 | A | — | 562 |
| 552 | H | 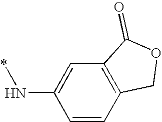 | I | — | 574 |

TABLE 28-7

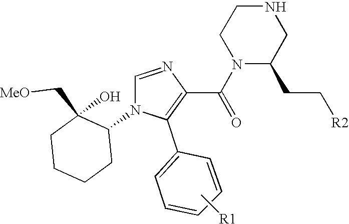

| Ex. No. | R1 | R2 | Method | Salt | MS (ESI+) |
|---|---|---|---|---|---|
| 553 | H | 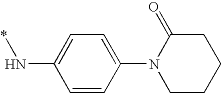 | I | — | 615 |
| 554 | H | 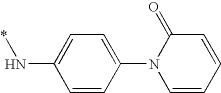 | I | — | 611 |
| 555 | H | 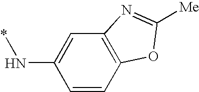 | I | — | 573 |
| 556 | H | 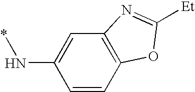 | I | — | 587 |
| 557 | H | 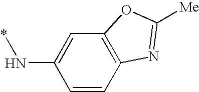 | L | — | 573 |
| 558 | H | 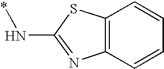 | L | — | 575 |
| 559 | H | 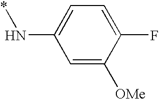 | A | — | 566 |
| 560 | H | 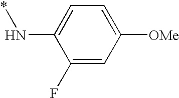 | A | — | 566 |
| 561 | H | 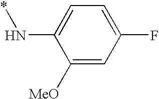 | A | — | 566 |
| 562 | H | 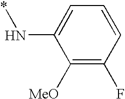 | A | — | 566 |
| 563 | H | 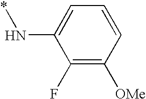 | A | — | 566 |
| 564 | H | 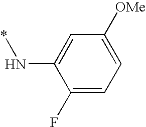 | A | — | 566 |

TABLE 28-8

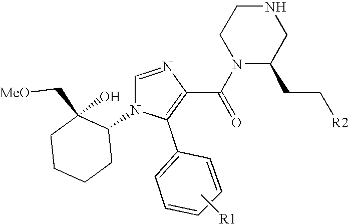

| Ex. | | | | | |
|---|---|---|---|---|---|
| No. | R1 | R2 | Method | Salt | MS (ESI+) |
| 565 | H | 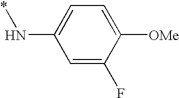 | A | 3HCl | 566 |
| 566 | H | 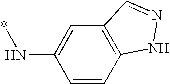 | I | — | 578 |
| 567 | H | 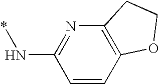 | I | — | 561 |

The chemical names of the compounds (Examples 430-567) shown in Table 25-1-Table 25-2, Table 26, Table 27-1-Table 27-2 and Table 28-1-Table 28-8 are as follows.
Example 430: Methyl {(1S,2S)-2-[5-phenyl-4-({(2R)-2-[2-(trifluoromethyl)benzyl]piperazin-1-yl}carbonyl)-1H-imidazol-1-yl]cyclohexyl}carbamate
Example 431: Methyl {(1S,2S)-2-[5-phenyl-4-({(2R)-2-[3-(trifluoromethyl)benzyl]piperazin-1-yl}carbonyl)-1H-imidazol-1-yl]cyclohexyl}carbamate
Example 432: Methyl {(1S,2S)-2-[5-phenyl-4-({(2R)-2-[4-(trifluoromethyl)benzyl]piperazin-1-yl}carbonyl)-1H-imidazol-1-yl]cyclohexyl}carbamate
Example 433: Methyl [(1S,2S)-2-(4-{[(2R)-2-(3,4-difluorobenzyl)piperazin-1-yl]carbonyl}-5-phenyl-1H-imidazol-1-yl)cyclohexyl]carbamate
Example 434: Methyl [(1S,2S)-2-(4-{[(2R)-2-(biphenyl-4-ylmethyl)piperazin-1-yl]carbonyl}-5-phenyl-1H-imidazol-1-yl)cyclohexyl]carbamate
Example 435: Methyl [(1S,2S)-2-(4-{[(2R)-2-(2,3-dihydro-1H-inden-2-yl)piperazin-1-yl]carbonyl}-5-phenyl-1H-imidazol-1-yl)cyclohexyl]carbamate
Example 436: Methyl 3-(2-{(2R)-1-[(1-{(1S,2S)-2-[(ethoxycarbonyl)amino]cyclohexyl}-5-phenyl-1H-imidazol-4-yl)carbonyl] piperazin-2-yl}ethoxy)benzoate
Example 437: Methyl {(1S,2S)-2-[4-({(2R)-2-[2-(2-fluoro-4-methoxyphenoxy)ethyl]piperazin-1-yl}carbonyl)-5-phenyl-1H-imidazol-1-yl]cyclohexyl}carbamate
Example 438: Ethyl {(1S,2S)-2-[4-({(2R)-2-[2-(2-fluoro-4-methoxyphenoxy)ethyl]piperazin-1-yl}carbonyl)-5-phenyl-1H-imidazol-1-yl]cyclohexyl}carbamate
Example 439: Methyl {(1S,2S)-2-[4-({(2R)-2-[2-(2-fluorophenoxy)ethyl]piperazin-1-yl}carbonyl)-5-phenyl-1H-imidazol-1-yl]cyclohexyl}carbamate
Example 440: Ethyl {(1S,2S)-2-[4-({(2R)-2-[2-(2-fluorophenoxy)ethyl]piperazin-1-yl}carbonyl)-5-phenyl-1H-imidazol-1-yl]cyclohexyl}carbamate
Example 441: Methyl {(1S,2S)-2-[4-({(2R)-2-[2-(3-fluorophenoxy)ethyl]piperazin-1-yl}carbonyl)-5-phenyl-1H-imidazol-1-yl]cyclohexyl}carbamate
Example 442: Ethyl {(1S,2S)-2-[4-({(2R)-2-[2-(3-fluorophenoxy)ethyl]piperazin-1-yl}carbonyl)-5-phenyl-1H-imidazol-1-yl]cyclohexyl}carbamate
Example 443: Methyl {(1S,2S)-2-[5-phenyl-4-({(2R)-2-[2-(phenylthio)ethyl]piperazin-1-yl}carbonyl)-1H-imidazol-1-yl]cyclohexyl}carbamate
Example 444: Methyl [(1S,2S)-2-(4-{[(2R)-2-(2-anilinoethyl)piperazin-1-yl]carbonyl}-5-phenyl-1H-imidazol-1-yl)cyclohexyl]carbamate
Example 445: Methyl ((1S,2S)-2-{4-[((2R)-2-{2-[(2-isopropylphenyl)amino]ethyl}piperazin-1-yl)carbonyl]-5-phenyl-1H-imidazol-1-yl}cyclohexyl)carbamate
Example 446: Methyl ((1S,2S)-2-{4-[((2R)-2-{2-[(2,4-difluorophenyl)amino]ethyl}piperazin-1-yl)carbonyl]-5-phenyl-1H-imidazol-1-yl}cyclohexyl)carbamate
Example 447: Methyl ((1S,2S)-2-{4-[((2R)-2-{2-[(3,5-difluorophenyl)amino]ethyl}piperazin-1-yl)carbonyl]-5-phenyl-1H-imidazol-1-yl}cyclohexyl)carbamate
Example 448: Ethyl ((1S,2S)-2-{4-[((2R)-2-{2-[(2-fluoro-4-methoxyphenyl)amino]ethyl}piperazin-1-yl)carbonyl]-5-phenyl-1H-imidazol-1-yl}cyclohexyl)carbamate
Example 449: Ethyl ((1R,2R)-2-{4-[((2R)-2-{2-[(2-fluoro-3-methoxyphenyl)amino]ethyl}piperazin-1-yl)carbonyl]-5-phenyl-1H-imidazol-1-yl}cyclohexyl)carbamate
Example 450: Methyl ((1S,2S)-2-{4-[((2R)-2-{2-[(2-fluorophenyl)(methyl)amino]ethyl}piperazin-1-yl)carbonyl]-5-phenyl-1H-imidazol-1-yl}cyclohexyl)carbamate
Example 451: (1S,2R)-2-(4-{[(2R)-2-Benzylpiperazin-1-yl]carbonyl}-5-phenyl-1H-imidazol-1-yl)-1-(ethoxymethyl)cyclohexanol dihydrochloride
Example 452: (1S,2R)-2-(4-{[(2R)-2-Benzylpiperazin-1-yl]carbonyl}-5-phenyl-1H-imidazol-1-yl)-1-methylcyclohexanol dihydrochloride
Example 453: (1S,2R)-2-(4-{[(2R)-2-Benzylpiperazin-1-yl]carbonyl}-5-phenyl-1H-imidazol-1-yl)-1-ethylcyclohexanol dihydrochloride
Example 454: (1R,2R)-1-(Cyclopropylmethyl)-2-(5-phenyl-4-{[(2R)-2-(pyridin-3-ylmethyl)piperazin-1-yl]carbonyl}-1H-imidazol-1-yl)cyclohexanol
Example 455: (1R,2R)-1-(Cyclopropylmethyl)-2-(5-phenyl-4-{[(2R)-2-(pyridin-4-ylmethyl)piperazin-1-yl]carbonyl}-1H-imidazol-1-yl)cyclohexanol
Example 456: (1S,2R)-1-Ethyl-2-[5-phenyl-4-({(2R)-2-[(5-phenyl-1,3,4-oxadiazol-2-yl)methyl]piperazin-1-yl}carbonyl)-1H-imidazol-1-yl]cyclohexanol dihydrochloride
Example 457: (1R,2R)-1-(Cyclopropylmethyl)-2-[5-phenyl-4-({(2R)-2-[(5-phenyl-1,3,4-oxadiazol-2-yl)methyl]piperazin-1-yl}carbonyl)-1H-imidazol-1-yl]cyclohexanol
Example 458: (1S,2R)-1-(Ethoxymethyl)-2-[5-phenyl-4-({(2R)-2-[(5-phenyl-1,3,4-oxadiazol-2-yl)methyl]piperazin-1-yl}carbonyl)-1H-imidazol-1-yl]cyclohexanol
Example 459: (1R,2R)-1-(Cyclopropylmethyl)-2-{4-[((2S)-2-{[(3-fluorophenyl)sulfonyl]methyl}piperazin-1-yl)carbonyl]-5-phenyl-1H-imidazol-1-yl}cyclohexanol
Example 460: (1S,2R)-2-[4-({(2R)-2-[2-(2-Fluorophenoxy)ethyl]piperazin-1-yl}carbonyl)-5-phenyl-1H-imidazol-1-yl]-1-methylcyclohexanol
Example 461: (1S,2R)-1-Methyl-2-{4-[((2R)-2-{2-[(2-methyl-1,3-benzothiazol-5-yl)oxy]ethyl}piperazin-1-yl)carbonyl]-5-phenyl-1H-imidazol-1-yl}cyclohexanol
Example 462: (1S,2R)-2-{4-[((2R)-2-{2-[(2-Fluoro-4-methoxyphenyl)amino]ethyl}piperazin-1-yl)carbonyl]-5-phenyl-1H-imidazol-1-yl}-1-methylcyclohexanol
Example 463: (1S,2R)-2-{4-[((2R)-2-{2-[(2-Fluoro-3-methoxyphenyl)amino]ethyl}piperazin-1-yl)carbonyl]-5-phenyl-1H-imidazol-1-yl}-1-methylcyclohexanol
Example 464: (1S,2R)-1-(Methoxymethyl)-2-(4-{[(2R)-2-(2-Naphthylmethyl)piperazin-1-yl]carbonyl}-5-phenyl-1H-imidazol-1-yl)cyclohexanol hydrochloride
Example 465: (1S,2R)-2-(4-{[(2R)-2-(Biphenyl-4-ylmethyl)piperazin-1-yl]carbonyl}-5-phenyl-1H-imidazol-1-yl)-1-(methoxymethyl)cyclohexanol hydrochloride
Example 466: [(2S)-1-({1-[(1R,2S)-2-Hydroxy-2-(methoxymethyl)cyclohexyl]-5-phenyl-1H-imidazol-4-yl}carbonyl)piperazin-2-yl]methyl phenylcarbamate
Example 467: (1S,2R)-1-(Methoxymethyl)-2-[5-phenyl-4-({(2S)-2-[(2-phenyl-1H-imidazol-1-yl)methyl]piperazin-1-yl}carbonyl)-1H-imidazol-1-yl]cyclohexanol trihydrochloride
Example 468: (1S,2R)-1-(Methoxymethyl)-2-[5-phenyl-4-({(2S)-2-[(5-phenyl-1H-imidazol-1-yl)methyl]piperazin-1-yl}carbonyl)-1H-imidazol-1-yl]cyclohexanol trihydrochloride
Example 469: (1S,2R)-2-(4-{[(2R)-2-(2-Methoxybenzyl)piperazin-1-yl]carbonyl}-5-phenyl-1H-imidazol-1-yl)-1-(methoxymethyl)cyclohexanol
Example 470: (1S,2R)-2-(4-{[(2S)-2-(2-Methoxybenzyl)piperazin-1-yl]carbonyl}-5-phenyl-1H-imidazol-1-yl)-1-(methoxymethyl)cyclohexanol
Example 471: (1S,2R)-2-(4-{[(2R)-2-(2,3-Dihydro-1H-inden-2-yl)piperazin-1-yl]carbonyl}-5-phenyl-1H-imidazol-1-yl)-1-(methoxymethyl)cyclohexanol
Example 472: (1S,2R)-2-(4-{[(2R)-2-(Biphenyl-2-ylmethyl)piperazin-1-yl]carbonyl}-5-phenyl-1H-imidazol-1-yl)-1-(methoxymethyl)cyclohexanol
Example 473: (1S,2R)-2-(4-{[(2S)-2-(Biphenyl-2-ylmethyl)piperazin-1-yl]carbonyl}-5-phenyl-1H-imidazol-1-yl)-1-(methoxymethyl)cyclohexanol
Example 474: (1S,2R)-1-(Methoxymethyl)-2-(4-{[(2R)-2-(2-morpholinobenzyl)piperazin-1-yl]carbonyl}-5-phenyl-1H-imidazol-1-yl)cyclohexanol
Example 475: (1S,2R)-1-(Methoxymethyl)-2-[4-({(2R)-2-[2-(2-methoxypyridin-3-yl)benzyl]piperazin-1-yl}carbonyl)-5-phenyl-1H-imidazol-1-yl]cyclohexanol
Example 476: (1S,2R)-1-(Methoxymethyl)-2-[4-({(2R)-2-[2-(1-methyl-1H-pyrazol-5-yl)benzyl]piperazin-1-yl}carbonyl)-5-phenyl-1H-imidazol-1-yl]cyclohexanol
Example 477: (1S,2R)-1-(Methoxymethyl)-2-[5-phenyl-4-({(2S)-2-[(phenylthio)methyl]piperazin-1-yl}carbonyl)-1H-imidazol-1-yl]cyclohexanol dihydrochloride
Example 478: (1S,2R)-1-(Methoxymethyl)-2-[5-phenyl-4-({(2S)-2-[(phenylsulfinyl)methyl]piperazin-1-yl}carbonyl)-1H-imidazol-1-yl]cyclohexanol
Example 479: (1S,2R)-1-(Methoxymethyl)-2-[5-phenyl-4-({(2S)-2-[(phenylsulfonyl)methyl]piperazin-1-yl}carbonyl)-1H-imidazol-1-yl]cyclohexanol dihydrochloride
Example 480: (1S,2R)-2-{4-[((2S)-2-{[(3-Fluorophenyl)sulfonyl]methyl}piperazin-1-yl)carbonyl]-5-phenyl-1H-imidazol-1-yl}-1-(methoxymethyl)cyclohexanol dihydrochloride
Example 481: 2-[(2R)-1-({1-[(1R,2S)-2-Hydroxy-2-(methoxymethyl)cyclohexyl]-5-phenyl-1H-imidazol-4-yl}carbonyl)piperazin-2-yl]-N-phenylacetamide
Example 482: 2-[(2R)-1-({1-[(1R,2S)-2-Hydroxy-2-(methoxymethyl)cyclohexyl]-5-phenyl-1H-imidazol-4-yl}carbonyl)piperazin-2-yl]-N-methyl-N-phenylacetamide
Example 483: (1S,2R)-2-{4-[((2S)-2-{[(2-Ethyl-1,3-benzoxazol-5-yl)amino]methyl}piperazin-1-yl)carbonyl]-5-phenyl-1H-imidazol-1-yl}-1-(methoxymethyl)cyclohexanol
Example 484: (1S,2R)-2-[4-({(2R)-2-[2-(1-Benzothien-4-yloxy)ethyl]piperazin-1-yl}carbonyl)-5-phenyl-1H-imidazol-1-yl]-1-(methoxymethyl)cyclohexanol
Example 485: (1S,2R)-1-(Methoxymethyl)-2-[4-({(2R)-2-[2-(2-methoxy-4-morpholinophenoxy)ethyl]piperazin-1-yl}carbonyl)-5-phenyl-1H-imidazol-1-yl]cyclohexanol dihydrochloride
Example 486: (1S,2R)-2-{4-[((2R)-2-{2-[4-(4-Acetylpiperazin-1-yl)-2-methoxyphenoxy]ethyl}piperazin-1-yl)carbonyl]-5-phenyl-1H-imidazol-1-yl}-1-(methoxymethyl)cyclohexanol dihydrochloride
Example 487: (1S,2R)-2-{4-[((2R)-2-{2-[3-(Difluoromethoxy)phenoxy]ethyl}piperazin-1-yl)carbonyl]-5-phenyl-1H-imidazol-1-yl}-1-(methoxymethyl)cyclohexanol hydrochloride
Example 488: 2-(4-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}phenyl)-5-methyl-1,2-dihydro-3H-imidazo[1,5-c]imidazol-3-one
Example 489: Ethyl 5-{2-[(2R)-1-({1-[(1R,2S)-2-hydroxy-2-(methoxymethyl)cyclohexyl]-5-phenyl-1H-imidazol-4-yl}carbonyl)piperazin-2-yl]ethoxy}-1-benzofuran-2-carboxylate
Example 490: (1S,2R)-2-{4-[((2R)-2-{2-[2-Fluoro-4-(1H-pyrazol-1-yl)phenoxy]ethyl}piperazin-1-yl)carbonyl]-5-phenyl-1H-imidazol-1-yl}-1-(methoxymethyl)cyclohexanol dihydrochloride
Example 491: (1S,2R)-1-(Methoxymethyl)-2-{4-[((2R)-2-{2-[2-methoxy-4-(1H-pyrazol-1-yl)phenoxy]ethyl}piperazin-1-yl)carbonyl]-5-phenyl-1H-imidazol-1-yl}cyclohexanol
Example 492: 1-(4-{2-[(2R)-1-({5-(3-Fluorophenyl)-1-[(1R,2S)-2-hydroxy-2-(methoxymethyl)cyclohexyl]-1H-imidazol-4-yl}carbonyl)piperazin-2-yl]ethoxy}phenyl)ethanone dihydrochloride
Example 493: (1S,2R)-1-(Methoxymethyl)-2-[4-({(2R)-2-[2-(2-methylphenoxy)ethyl]piperazin-1-yl}carbonyl)-5-phenyl-1H-imidazol-1-yl]cyclohexanol
Example 494: (1S,2R)-1-(Methoxymethyl)-2-[4-({(2R)-2-[2-(4-methoxy-2-methylphenoxy)ethyl]piperazin-1-yl}carbonyl)-5-phenyl-1H-imidazol-1-yl]cyclohexanol
Example 495: (1S,2R)-2-[4-({(2R)-2-[2-(2,3-Dihydro-1-benzofuran-5-yloxy)ethyl]piperazin-1-yl}carbonyl)-5-phenyl-1H-imidazol-1-yl]-1-(methoxymethyl)cyclohexanol
Example 496: (1S,2R)-2-{4-[((2R)-2-{2-[(2,2-Dimethyl-2,3-dihydro-1-benzofuran-5-yl)oxyl]ethyl}piperazin-1-yl)carbonyl]-5-phenyl-1H-imidazol-1-yl}-1-(methoxymethyl)cyclohexanol
Example 497: (1S,2R)-2-{4-[((2R)-2-{2-[(7-Fluoro-2,2-dimethyl-2,3-dihydro-1-benzofuran-5-yl)oxy]ethyl}piperazin-1-yl)carbonyl]-5-phenyl-1H-imidazol-1-yl}-1-(methoxymethyl)cyclohexanol
Example 498: (1S,2R)-2-{4-[((2R)-2-{2-[(1,2-Dimethyl-1H-benzimidazol-5-yl)oxy]ethyl}piperazin-1-yl)carbonyl]-5-phenyl-1H-imidazol-1-yl}-1-(methoxymethyl)cyclohexanol
Example 499: 2-[(2R)-1-({1-[(1R,2S)-2-Hydroxy-2-(methoxymethyl)cyclohexyl]-5-phenyl-1H-imidazol-4-yl}carbonyl)piperazin-2-yl]ethyl piperidine-1-carboxylate dihydrochloride
Example 500: 2-[(2R)-1-({1-[(1R,2S)-2-Hydroxy-2-(methoxymethyl)cyclohexyl]-5-phenyl-1H-imidazol-4-yl}carbonyl)piperazin-2-yl]ethyl phenylcarbamate dihydrochloride
Example 501: (1S,2R)-1-(Methoxymethyl)-2-[5-phenyl-4-({(2R)-2-[2-(phenylthio)ethyl]piperazin-1-yl}carbonyl)-1H-imidazol-1-yl]cyclohexanol dihydrochloride
Example 502: (1S,2R)-1-(Methoxymethyl)-2-[5-phenyl-4-({(2R)-2-[2-(phenylsulfinyl)ethyl]piperazin-1-yl}carbonyl)-1H-imidazol-1-yl]cyclohexanol
Example 503: (1S,2R)-1-(Methoxymethyl)-2-[5-phenyl-4-({(2R)-2-[2-(phenylsulfonyl)ethyl]piperazin-1-yl}carbonyl)-1H-imidazol-1-yl]cyclohexanol dihydrochloride
Example 504: (1S,2R)-2-[4-({(2R)-2-[2-(1,3-Benzothiazol-2-ylthio)ethyl]piperazin-1-yl}carbonyl)-5-phenyl-1H-imidazol-1-yl]-1-(methoxymethyl)cyclohexanol
Example 505: (1S,2R)-1-(Methoxymethyl)-2-[5-phenyl-4-({(2R)-2-[2-([1,3]thiazolo[5,4-b]pyridin-2-ylthio)ethyl]piperazin-1-yl}carbonyl)-1H-imidazol-1-yl]cyclohexanol
Example 506: (1S,2R)-1-(Methoxymethyl)-2-{4-[((2R)-2-{2-[(4-methyl-1,3-thiazol-2-yl)thio]ethyl}piperazin-1-yl)carbonyl]-5-phenyl-1H-imidazol-1-yl}cyclohexanol
Example 507: (1S,2R)-2-{4-[((2R)-2-{2-[(4-tert-Butyl-1,3-thiazol-2-yl)thio]ethyl}piperazin-1-yl)carbonyl]-5-phenyl-1H-imidazol-1-yl}-1-(methoxymethyl)cyclohexanol
Example 508: (1S,2R)-2-{4-[((2R)-2-{2-[(4,5-Dimethyl-1,3-thiazol-2-yl)thio]ethyl}piperazin-1-yl)carbonyl]-5-phenyl-1H-imidazol-1-yl}-1-(methoxymethyl)cyclohexanol
Example 509: (1S,2R)-1-(Methoxymethyl)-2-{4-[((2R)-2-{2-[(5-methyl-1,3,4-thiadiazol-2-yl)thio]ethyl}piperazin-1-yl)carbonyl]-5-phenyl-1H-imidazol-1-yl}cyclohexanol
Example 510: (1S,2R)-2-[4-({(2R)-2-[2-(1H-Benzimidazol-2-ylthio)ethyl]piperazin-1-yl}carbonyl)-5-phenyl-1H-imidazol-1-yl]-1-(methoxymethyl)cyclohexanol
Example 511: (1S,2R)-1-(Methoxymethyl)-2-{4-[((2R)-2-{2-[(4-methyl-4H-1,2,4-triazol-3-yl)thio]ethyl}piperazin-1-yl)carbonyl]-5-phenyl-1H-imidazol-1-yl}cyclohexanol
Example 512: N-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethyl}-N-phenylacetamide
Example 513: N-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethyl}-N-phenylcyclopropanecarboxamide
Example 514: (1S,2R)-2-[4-({(2R)-2-[2-(1,3-Dihydro-2H-isoindol-2-yl)ethyl]piperazin-1-yl}carbonyl)-5-phenyl-1H-imidazol-1-yl]-1-(methoxymethyl)cyclohexanol trihydrochloride
Example 515: (1S,2R)-2-[4-({(2R)-2-[2-(3,4-Dihydroisoquinolin-2(1H)-yl)ethyl]piperazin-1-yl}carbonyl)-5-phenyl-1H-imidazol-1-yl]-1-(methoxymethyl)cyclohexanol trihydrochloride
Example 516: (1S,2R)-2-[4-({(2R)-2-[2-(3,4-Dihydroquinolin-1(2H)-yl)ethyl]piperazin-1-yl}carbonyl)-5-phenyl-1H-imidazol-1-yl]-1-(methoxymethyl)cyclohexanol trihydrochloride
Example 517: (1S,2R)-1-(Methoxymethyl)-2-[5-phenyl-4-({(2R)-2-[2-(pyridin-2-ylamino)ethyl]piperazin-1-yl}carbonyl)-1H-imidazol-1-yl]cyclohexanol
Example 518: (1S,2R)-1-(Methoxymethyl)-2-(5-phenyl-4-{[(2R)-2-(2-{[2-(trifluoromethyl)phenyl]amino}ethyl)piperazin-1-yl]carbonyl}-1H-imidazol-1-yl)cyclohexanol
Example 519: 2-({2-[(2R)-1-({1-[(1R,2S)-2-Hydroxy-2-(methoxymethyl)cyclohexyl]-5-phenyl-1H-imidazol-4-yl}carbonyl)piperazin-2-yl]ethyl}amino)benzonitrile
Example 520: (1S,2R)-2-{4-[((2R)-2-{2-[Benzyl(cyclopropyl)amino]ethyl}piperazin-1-yl)carbonyl]-5-phenyl-1H-imidazol-1-yl}-1-(methoxymethyl)cyclohexanol
Example 521: (1S,2R)-2-{4-[((2R)-2-{2-[(2-Fluorophenyl)amino]ethyl}piperazin-1-yl)carbonyl]-5-phenyl-1H-imidazol-1-yl}-1-(methoxymethyl)cyclohexanol
Example 522: (1S,2R)-2-{4-[((2R)-2-{2-[(3-Fluorophenyl)amino]ethyl}piperazin-1-yl)carbonyl]-5-phenyl-1H-imidazol-1-yl}-1-(methoxymethyl)cyclohexanol
Example 523: (1S,2R)-2-{4-[((2R)-2-{2-[(4-Fluorophenyl)amino]ethyl}piperazin-1-yl)carbonyl]-5-phenyl-1H-imidazol-1-yl}-1-(methoxymethyl)cyclohexanol
Example 524: (1S,2R)-2-{4-[((2R)-2-{2-[(2-Chlorophenyl)amino]ethyl}piperazin-1-yl)carbonyl]-5-phenyl-1H-imidazol-1-yl}-1-(methoxymethyl)cyclohexanol
Example 525: (1S,2R)-1-(Methoxymethyl)-2-{4-[((2R)-2-{2-[(2-nitrophenyl)amino]ethyl}piperazin-1-yl)carbonyl]-5-phenyl-1H-imidazol-1-yl}cyclohexanol
Example 526: (1S,2R)-1-(Methoxymethyl)-2-{4-[((2R)-2-{2-[(2-methylphenyl)amino]ethyl}piperazin-1-yl)carbonyl]-5-phenyl-1H-imidazol-1-yl}cyclohexanol
Example 527: (1S,2R)-1-(Methoxymethyl)-2-{4-[((2R)-2-{2-[(4-methylphenyl)amino]ethyl}piperazin-1-yl)carbonyl]-5-phenyl-1H-imidazol-1-yl}cyclohexanol
Example 528: (1S,2R)-1-(Methoxymethyl)-2-{4-[((2R)-2-{2-[(2-methoxyphenyl)amino]ethyl}piperazin-1-yl)carbonyl]-5-phenyl-1H-imidazol-1-yl}cyclohexanol
Example 529: (1S,2R)-1-(Methoxymethyl)-2-{4-[((2R)-2-{2-[(3-methoxyphenyl)amino]ethyl}piperazin-1-yl)carbonyl]-5-phenyl-1H-imidazol-1-yl}cyclohexanol
Example 530: (1S,2R)-1-(Methoxymethyl)-2-{4-[((2R)-2-{2-[(4-methoxyphenyl)amino]ethyl}piperazin-1-yl)carbonyl]-5-phenyl-1H-imidazol-1-yl}cyclohexanol
Example 531: Methyl 4-({2-[(2R)-1-({1-[(1R,2S)-2-hydroxy-2-(methoxymethyl)cyclohexyl]-5-phenyl-1H-imidazol-4-yl}carbonyl)piperazin-2-yl]ethyl}amino)benzoate
Example 532: 1-[4-({2-[(2R)-1-({1-[(1R,2S)-2-Hydroxy-2-(methoxymethyl)cyclohexyl]-5-phenyl-1H-imidazol-4-yl}carbonyl)piperazin-2-yl]ethyl}amino)phenyl]ethanone
Example 533: Methyl 3-({2-[(2R)-1-({1-[(1R,2S)-2-hydroxy-2-(methoxymethyl)cyclohexyl]-5-phenyl-1H-imidazol-4-yl}carbonyl)piperazin-2-yl]ethyl}amino)benzoate
Example 534: 1-[3-({2-[(2R)-1-({1-[(1R,2S)-2-Hydroxy-2-(methoxymethyl)cyclohexyl]-5-phenyl-1H-imidazol-4-yl}carbonyl)piperazin-2-yl]ethyl}amino)phenyl]ethanone
Example 535: (1S,2R)-1-(Methoxymethyl)-2-(5-phenyl-4-{[(2R)-2-(2-{[4-(trifluoromethoxy)phenyl]amino}ethyl)piperazin-1-yl]carbonyl}-1H-imidazol-1-yl)cyclohexanol
Example 536: (1S,2R)-2-(4-{[(2R)-2-(2-{[2-(Difluoromethoxy)phenyl]amino}ethyl)piperazin-1-yl]carbonyl}-5-phenyl-1H-imidazol-1-yl--1-(methoxymethyl)cyclohexanol
Example 537: (1S,2R)-2-(4-{[(2R)-2-(2-{[3-(Difluoromethoxy)phenyl]amino}ethyl)piperazin-1-yl]carbonyl}-5-phenyl-1H-imidazol-1-yl)-1-(methoxymethyl)cyclohexanol
Example 538: (1S,2R)-2-(4-{[(2R)-2-(2-{[4-(Difluoromethoxy)phenyl]amino}ethyl)piperazin-1-yl]carbonyl}-5-phenyl-1H-imidazol-1-yl)-1-(methoxymethyl)cyclohexanol
Example 539: (1S,2R)-1-(Methoxymethyl)-2-(4-{[(2R)-2-(2-{[2-methoxy-5-(trifluoromethyl)phenyl]amino}ethyl)piperazin-1-yl]carbonyl}-5-phenyl-1H-imidazol-1-yl)cyclohexanol
Example 540: (1S,2R)-2-[4-({(2R)-2-[2-(2,3-Dihydro-1H-inden-4-ylamino)ethyl]piperazin-1-yl}carbonyl)-5-phenyl-1H-imidazol-1-yl]-1-(methoxymethyl)cyclohexanol
Example 541: (1S,2R)-2-[4-({(2R)-2-[2-(1,3-Benzodioxol-5-ylamino)ethyl]piperazin-1-yl}carbonyl)-5-phenyl-1H-imidazol-1-yl]-1-(methoxymethyl)cyclohexanol
Example 542: Methyl 4-chloro-3-({2-[(2R)-1-({1-[(1R,2S)-2-hydroxy-2-(methoxymethyl)cyclohexyl]-5-phenyl-1H-imidazol-4-yl}carbonyl)piperazin-2-yl]ethyl}amino)benzoate
Example 543: 5-({2-[(2R)-1-({1-[(1R,2S)-2-Hydroxy-2-(methoxymethyl)cyclohexyl]-5-phenyl-1H-imidazol-4-yl}carbonyl)piperazin-2-yl]ethyl}amino)-2-benzofuran-1(3H)-one
Example 544: (1S,2R)-1-(Methoxymethyl)-2-(5-phenyl-4-{[(2R)-2-(2-{[4-(1H-pyrazol-1-yl)phenyl]amino}ethyl)piperazin-1-yl]carbonyl}-1H-imidazol-1-yl)cyclohexanol
Example 545: (1S,2R)-1-(Methoxymethyl)-2-{4-[((2R)-2-{2-[(4-methoxy-2-methylphenyl)amino]ethyl}piperazin-1-yl)carbonyl]-5-phenyl-1H-imidazol-1-yl}cyclohexanol
Example 546: (1S,2R)-1-(Methoxymethyl)-2-{4-[((2R)-2-{2-[(5-methoxy-2-methylphenyl)amino]ethyl}piperazin-1-yl)carbonyl]-5-phenyl-1H-imidazol-1-yl}cyclohexanol
Example 547: (1S,2R)-1-(Methoxymethyl)-2-{4-[((2R)-2-{2-[(2-methoxy-6-methylphenyl)amino]ethyl}piperazin-1-yl)carbonyl]-5-phenyl-1H-imidazol-1-yl}cyclohexanol
Example 548: (1S,2R)-1-(Methoxymethyl)-2-{4-[((2R)-2-{2-[(2-methoxy-4-methylphenyl)amino]ethyl}piperazin-1-yl)carbonyl]-5-phenyl-1H-imidazol-1-yl}cyclohexanol
Example 549: (1S,2R)-1-(Methoxymethyl)-2-{4-[((2R)-2-{2-[(2-methoxy-5-methylphenyl)amino]ethyl}piperazin-1-yl)carbonyl]-5-phenyl-1H-imidazol-1-yl}cyclohexanol
Example 550: (1S,2R)-1-(Methoxymethyl)-2-{4-[((2R)-2-{2-[(3-methoxy-4-methylphenyl)amino]ethyl}piperazin-1-yl)carbonyl]-5-phenyl-1H-imidazol-1-yl}cyclohexanol
Example 551: (1S,2R)-1-(Methoxymethyl)-2-{4-[((2R)-2-{2-[(3-methoxy-2-methylphenyl)amino]ethyl}piperazin-1-yl)carbonyl]-5-phenyl-1H-imidazol-1-yl}cyclohexanol
Example 552: 6-({2-[(2R)-1-({1-[(1R,2S)-2-Hydroxy-2-(methoxymethyl)cyclohexyl]-5-phenyl-1H-imidazol-4-yl}carbonyl)piperazin-2-yl]ethyl}amino)-2-benzofuran-1(3H)-one
Example 553: 1-[4-({2-[(2R)-1-({1-[(1R,2S)-2-Hydroxy-2-(methoxymethyl)cyclohexyl]-5-phenyl-1H-imidazol-4-yl}carbonyl)piperazin-2-yl]ethyl}amino)phenyl]piperidin-2-one
Example 554: 1-[4-({2-[(2R)-1-({1-[(1R,2S)-2-Hydroxy-2-(methoxymethyl)cyclohexyl]-5-phenyl-1H-imidazol-4-yl}carbonyl)piperazin-2-yl]ethyl}amino)phenyl]pyridin-2(1H)-one
Example 555: (1S,2R)-1-(Methoxymethyl)-2-{4-[((2R)-2-{2-[(2-methyl-1,3-benzoxazol-5-yl)amino]ethyl}piperazin-1-yl)carbonyl]-5-phenyl-1H-imidazol-1-yl}cyclohexanol
Example 556: (1S,2R)-2-{4-[((2R)-2-{2-[(2-ethyl-1,3-benzoxazol-5-yl)amino]ethyl}piperazin-1-yl)carbonyl]-5-phenyl-1H-imidazol-1-yl}-1-(methoxymethyl)cyclohexanol
Example 557: (1S,2R)-1-(Methoxymethyl)-2-{4-[((2R)-2-{2-[(2-methyl-1,3-benzoxazol-6-yl)amino]ethyl}piperazin-1-yl)carbonyl]-5-phenyl-1H-imidazol-1-yl}cyclohexanol
Example 558: (1S,2R)-2-[4-({(2R)-2-[2-(1,3-Benzothiazol-2-ylamino)ethyl]piperazin-1-yl}carbonyl)-5-phenyl-1H-imidazol-1-yl]-1-(methoxymethyl)cyclohexanol
Example 559: (1S,2R)-2-{4-[((2R)-2-{2-[(4-Fluoro-3-methoxyphenyl)amino]ethyl}piperazin-1-yl)carbonyl]-5-phenyl-1H-imidazol-1-yl}-1-(methoxymethyl)cyclohexanol
Example 560: (1S,2R)-2-{4-[((2R)-2-{2-[(2-Fluoro-4-methoxyphenyl)amino]ethyl}piperazin-1-yl)carbonyl]-5-phenyl-1H-imidazol-1-yl}-1-(methoxymethyl)cyclohexanol
Example 561: (1S,2R)-2-{4-[((2R)-2-{2-[(4-Fluoro-2-methoxyphenyl)amino]ethyl}piperazin-1-yl)carbonyl]-5-phenyl-1H-imidazol-1-yl}-1-(methoxymethyl)cyclohexanol
Example 562: (1S,2R)-2-{4-[((2R)-2-{2-[(3-Fluoro-2-methoxyphenyl)amino]ethyl}piperazin-1-yl)carbonyl]-5-phenyl-1H-imidazol-1-yl}-1-(methoxymethyl)cyclohexanol
Example 563: (1S,2R)-2-{4-[((2R)-2-{2-[(2-Fluoro-3-methoxyphenyl)amino]ethyl}piperazin-1-yl)carbonyl]-5-phenyl-1H-imidazol-1-yl}-1-(methoxymethyl)cyclohexanol
Example 564: (1S,2R)-2-{4-[((2R)-2-{2-[(2-Fluoro-5-methoxyphenyl)amino]ethyl}piperazin-1-yl)carbonyl]-5-phenyl-1H-imidazol-1-yl}-1-(methoxymethyl)cyclohexanol
Example 565: (1S,2R)-2-{4-[((2R)-2-{2-[(3-Fluoro-4-methoxyphenyl)amino]ethyl}piperazin-1-yl)carbonyl]-5-phenyl-1H-imidazol-1-yl}-1-(methoxymethyl)cyclohexanol trihydrochloride
Example 566: (1S,2R)-2-[4-({(2R)-2-[2-(1H-Indazol-5-ylamino)ethyl]piperazin-1-yl}carbonyl)-5-phenyl-1H-imidazol-1-yl]-1-(methoxymethyl)cyclohexanol
Example 567: (1S,2R)-2-[4-({(2R)-2-[2-(2,3-Dihydrofuro[3,2-b]pyridin-5-ylamino)ethyl]piperazin-1-yl}carbonyl)-5-phenyl-1H-imidazol-1-yl]-1-(methoxymethyl)cyclohexanol

In the same manner as in Example 1 (Method A) except that the treatment of the final product with 4N hydrogen chloride-ethyl acetate solution was omitted, the following compound (Example 568) was obtained as a free amorphous solid.

### Example 568

### (1S,2R)-2-(4-{[(2R)-2-Benzyl-3-methylpiperazin-1-yl]carbonyl}-5-phenyl-1H-imidazol-1-yl)-1-(methoxymethyl)cyclohexanol

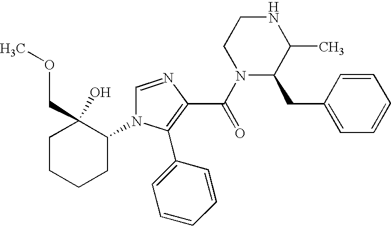

MS (ESI+, m/e) 503 (M+1)

In the same manner as in Example 6 (Method F), the following compounds (Examples 569-572) were obtained. The compound of Example 572 was isolated as a 2 TFA salt by subjecting the final product to reversed-phase preparative HPLC (the purification conditions are described above), and directly concentrating the object fraction under reduced pressure.

### Example 569

### 1-{[5-Phenyl-4-({(2R)-2-[(5-phenyl-1,3,4-oxadiazol-2-yl)methyl]piperazin-1-yl}carbonyl)-1H-imidazol-1-yl]methyl}cyclohexanol

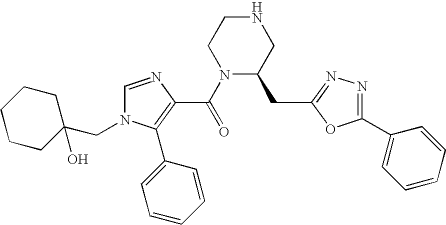

MS (ESI+, m/e) 527 (M+1)

### Example 570

### 1-({4-[((2R)-2-{2-[(2-Fluoro-4-methoxyphenyl)amino]ethyl}piperazin-1-yl)carbonyl]-5-phenyl-1H-imidazol-1-yl}methyl)cyclohexanol

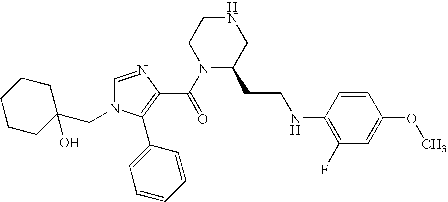

MS (ESI+, m/e) 536 (M+1)

### Example 571

### 1-({4-[((2R)-2-{2-[(2-Fluoro-3-methoxyphenyl)amino]ethyl}piperazin-1-yl)carbonyl]-5-phenyl-1H-imidazol-1-yl}methyl)cyclohexanol

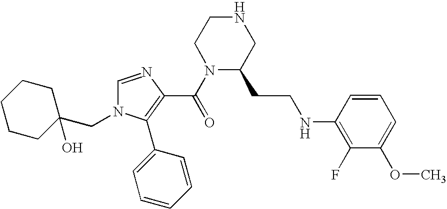

MS (ESI+, m/e) 536 (M+1)

### Example 572

### (1S,2R)-1-(Methoxymethyl)-2-(4-{[(2R)-2-(2-phenoxybenzyl)piperazin-1-yl]carbonyl}-5-phenyl-1H-imidazol-1-yl)cyclohexanol bistrifluoroacetate

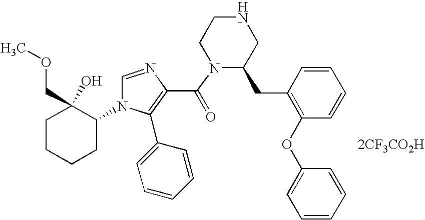

MS (ESI+, m/e) 581 (M+1)

### Example 573

### 1-[(4-{[(2R)-2-(2-Anilinoethyl)piperazin-1-yl]carbonyl}-5-phenyl-1H-imidazol-1-yl)methyl]cyclohexanol

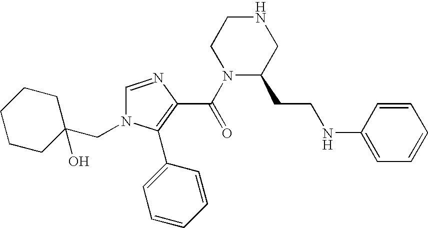

A mixture of ethyl 1-[(1-hydroxycyclohexyl)methyl]-5-phenyl-1H-imidazole-4-carboxylate (100 mg), lithium hydroxide monohydrate (20 mg), ethanol (3 ml) and water (1 ml) was stirred at 80° C. for 3 hr, and concentrated under reduced pressure. The residue was mixed with benzyl (3R)-3-(2-anilinoethyl)piperazine-1-carboxylate (109 mg), WSC.HCl (115 mg), HOBt (230 mg) and DMF (4 ml). The mixture was stirred at 50° C. for 5 hr, and poured into saturated aqueous sodium hydrogen carbonate, and the mixture was extracted with ethyl acetate. The extract was washed with saturated brine, and dried over anhydrous magnesium sulfate, and the solvent was evaporated under reduced pressure. The residue was subjected to silica gel column chromatography, and the fraction eluted with ethyl acetate-hexane-methanol (1:9:0-17:0:3) was concentrated under reduced pressure to give benzyl (3R)-3-(2-anilinoethyl)-4-({1-[(1-hydroxycyclohexyl)methyl]-5-phenyl-1H-imidazol-4-yl}carbonyl)piperazine-1-carboxylate (116 mg) as an amorphous solid. The total amount thereof was dissolved in ethanol (2 ml), 4N aqueous sodium hydroxide solution (2 ml) was added, and the mixture was stirred at 65° C. for 5 hr. The reaction mixture was concentrated under reduced pressure, water was added to the residue, and the liberated oil was extracted with ethyl acetate. The extract was washed with saturated brine, and dried over anhydrous sodium sulfate, and the solvent was evaporated under reduced pressure. The residue was subjected to basic silica gel column chromatography, and the fraction eluted with ethyl acetate-hexane (1:1-1:0) was concentrated under reduced pressure to give the object compound (55 mg) as an amorphous solid.

MS (ESI+, m/e) 488 (M+1)

### Example 574

### Methyl [(1S,2S)-2-(4-{[(2R)-2-(2-bromobenzyl)piperazin-1-yl]carbonyl}-5-phenyl-1H-imidazol-1-yl)cyclohexyl]carbamate

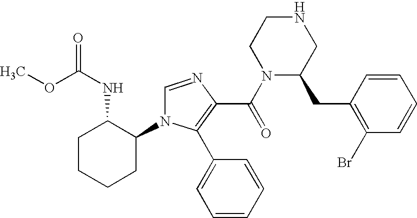

Methyl [(1S,2S)-2-(4-{[(2R)-4-benzyl-2-(2-bromobenzyl)piperazin-1-yl]carbonyl}-5-phenyl-1H-imidazol-1-yl)cyclohexyl]carbamate (671 mg) was dissolved in 1,2-dichloroethane (15 ml), 1-chloroethyl chloroformate (715 mg) was added, and the mixture was heated under reflux for 8 hr, and concentrated under reduced pressure. To the residue was added methanol (15 ml), and the mixture was further heated under reflux for 15 hr. The reaction mixture was concentrated under reduced pressure, to the residue saturated was added aqueous sodium hydrogen carbonate, and the mixture was extracted with ethyl acetate. The extract was washed with saturated brine, dried over anhydrous magnesium sulfate, and concentrated under reduced pressure. The residue was subjected to silica gel column chromatography, and the fraction eluted with ethyl acetate-methanol (4:1) was concentrated under reduced pressure to give the object compound (204 mg) as an amorphous solid.

MS (ESI+, m/e) 580 (M+1)

In the same manner as in Example 574, the following compound (Example 575) was obtained.

### Example 575

### (1S,2R)-2-(4-{[(2R)-2-(2-Bromobenzyl)piperazin-1-yl]carbonyl}-5-phenyl-1H-imidazol-1-yl)-1-(methoxymethyl)cyclohexanol

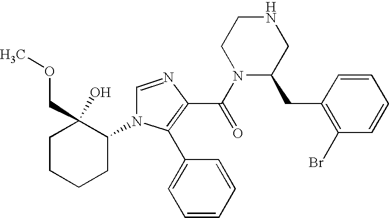

MS (ESI+, m/e) 567 (M+1)

In the same manner as in Example 382 except that the treatment of the final product with 4N hydrogen chloride-ethyl acetate solution was omitted, the following compound (Example 576) was obtained as an amorphous solid.

### Example 576

### Methyl [(1S,2S)-2-(5-phenyl-4-{[(2R)-2-(3-phenylpropyl)piperazin-1-yl]carbonyl}-1H-imidazol-1-yl)cyclohexyl]carbamate

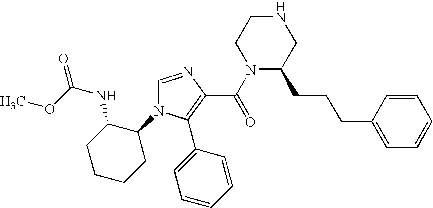

MS (ESI+, m/e) 530 (M+1)

### Example 577

### (1S,2R)-2-{4-[((2R)-2-{2-[Cyclohexyl(methyl)amino]ethyl}piperazin-1-yl)carbonyl]-5-phenyl-1H-imidazol-1-yl}-1-(methoxymethyl)cyclohexanol

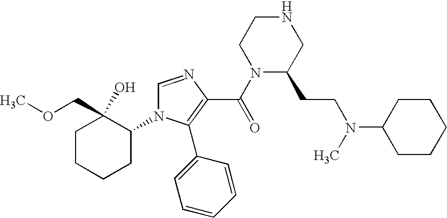

Benzyl (3R)-4-({1-[(1R,2S)-2-hydroxy-2-(methoxymethyl)cyclohexyl]-5-phenyl-1H-imidazol-4-yl}carbonyl)-3-{2-[methyl(phenyl)amino]ethyl}piperazine-1-carboxylate obtained in the course of Example 429 (150 mg) was dissolved in methanol (5 ml), 20% palladium hydroxide-carbon (50% containing water, 70 mg) was added thereto, and the mixture was subjected to catalytic reduction at ambient temperature and normal pressure for 12 hr. The catalyst was filtered off, and the filtrate was concentrated under reduced pressure. The residue was subjected to basic silica gel column chromatography, and the fraction eluted with ethyl acetate-methanol (1:0-9:2) was concentrated under reduced pressure to give the object compound (75 mg) as an amorphous solid.

MS (ESI+, m/e) 538 (M+1)

In the same manner as in Example 416, the following compounds (Examples 578-580) were obtained. The compounds of Examples 579-580 were isolated as free amorphous solids by extracting the final product with ethyl acetate and subjecting the extract to basic silica gel column chromatography.

### Example 578

### 3-({2-[(2R)-1-({1-[(1R,2S)-2-Hydroxy-2-(methoxymethyl)cyclohexyl]-5-phenyl-1H-imidazol-4-yl}carbonyl)piperazin-2-yl]ethyl}amino)benzoic acid tri-trifluoroacetate

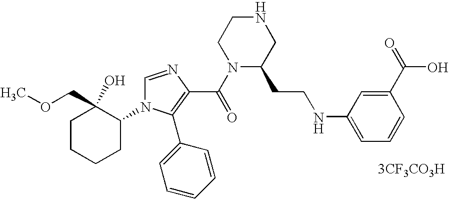

MS (ESI+, m/e) 562 (M+1)

### Example 579

### (1S,2R)-1-(Methoxymethyl)-2-{4-[((2R)-2-{2-[(2-methyl-1,3-benzothiazol-5-yl)amino]ethyl}piperazin-1-yl)carbonyl]-5-phenyl-1H-imidazol-1-yl}cyclohexanol

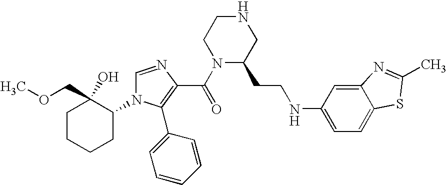

MS (ESI+, m/e) 589 (M+1)

### Example 580

### (1S,2R)-1-(Methoxymethyl)-2-{4-[((2R)-2-{2-[(2-methyl-1,3-benzothiazol-6-yl)amino]ethyl}piperazin-1-yl)carbonyl]-5-phenyl-1H-imidazol-1-yl}cyclohexanol

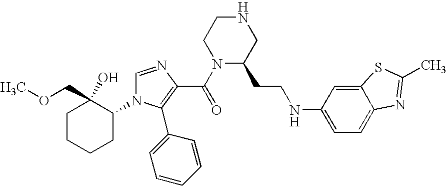

MS (ESI+, m/e) 589 (M+1)

### Example 581

### (1S,2R)-1-(Methoxymethyl)-2-(5-phenyl-4-{[(2R)-2-(2-(pyridin-2-yl)benzyl)piperazin-1-yl]carbonyl}-1H-imidazol-1-yl)cyclohexanol

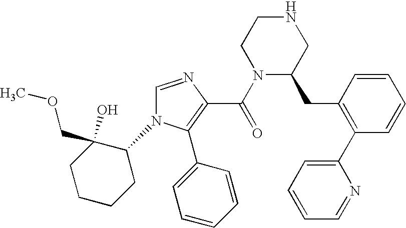

tert-Butyl (2R)-4-benzyl-2-(2-(pyridin-2-yl)benzyl)piperazine-1-carboxylate (140 mg) was dissolved in ethyl acetate (1 ml), 4N hydrogen chloride-ethyl acetate solution (1 ml) was added, and the mixture was stirred at room temperature for 1 hr. The reaction mixture was concentrated under reduced pressure, the residue was suspended in toluene (1 ml), and the suspension was again concentrated under reduced pressure. The residue was suspended in DMF (2 ml), 1-[(1R,2S)-2-hydroxy-2-(methoxymethyl)cyclohexyl]-5-phenyl-1H-imidazole-4-carboxylic acid (96 mg), WSC.HCl (83 mg), HOBt (67 mg), triethylamine (187 mg) were added, and the suspension was stirred at room temperature for 12 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to basic silica gel column chromatography, and the fraction eluted with ethyl acetate-hexane (1:1-1:0) was concentrated under reduced pressure to give (1S,2R)-2-[4-({(2R)-4-benzyl-2-[2-(6-chloropyridin-2-yl)benzyl]piperazin-1-yl}carbonyl)-5-phenyl-1H-imidazol-1-yl]-1-(methoxymethyl)cyclohexanol (115 mg) as an amorphous solid. The total amount thereof was dissolved in methanol (3 ml), 20% palladium hydroxide-carbon (50% containing water, 60 mg) was added thereto, and the mixture was subjected to catalytic reduction at ambient temperature and normal pressure for 6 hr. The catalyst was filtered off, and the filtrate was concentrated under reduced pressure to give the object compound (67 mg). (During the catalytic reduction, the removal of the chlorine atom proceeded together with the removal of the benzyl protecting group.)

MS (ESI+, m/e) 566 (M+1)

### Example 582

### (1S,2S)-2-{4-[((2R)-2-{2-[(2-Methyl-1,3-benzothiazol-5-yl)oxy]ethyl}piperazin-1-yl)carbonyl]-5-phenyl-1H-imidazol-1-yl}cyclohexanamine

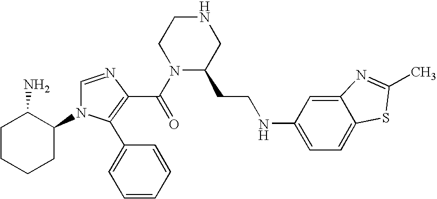

Benzyl (3R)-3-{2-[(2-methyl-1,3-benzothiazol-5-yl)oxy]ethyl}piperazine-1-carboxylate (200 mg) was dissolved in DMF (30 ml), 1-{(1S,2S)-2-[(methoxycarbonyl)amino]cyclohexyl}-5-phenyl-1H-imidazole-4-carboxylic acid (168 mg), WSC.HCl (142 mg), HOBt (93 mg) and N,N-diisopropylethylamine (253 μl) were added thereto, and the mixture was stirred at room temperature for 15 hr. The reaction mixture was poured into saturated aqueous sodium hydrogen carbonate, and the mixture was extracted with ethyl acetate. The extract was washed successively with water and saturated brine, and dried over anhydrous sodium sulfate, and the solvent was evaporated under reduced pressure. The residue was subjected to basic silica gel column chromatography, and the fraction eluted with ethyl acetate-methanol (9:1) was concentrated under reduced pressure to give benzyl (3R)-4-[(1-{(1S,2S)-2-[(methoxycarbonyl)amino]cyclohexyl}-5-phenyl-1H-imidazol-4-yl)carbonyl]-3-{2-[(2-methyl-1,3-benzothiazol-5-yl)oxy]ethyl}piperazine-1-carboxylate (100 mg) as an amorphous solid. The total amount thereof was dissolved in 25% hydrogen bromide-acetic acid solution (2 ml), and the mixture was stirred at room temperature for 3 hr. The reaction mixture was poured into water, and the mixture was washed with ethyl acetate. To the aqueous layer was added potassium carbonate by small portions to basify the layer, and the mixture was saturated with sodium chloride, and extracted with ethyl acetate. The extract was washed with saturated brine, and dried over anhydrous magnesium sulfate, and the solvent was evaporated under reduced pressure. The residue was subjected to basic silica gel column chromatography, and the fraction eluted with ethyl acetate-methanol (4:1) was concentrated under reduced pressure to give the object compound (11 mg) as an amorphous solid.

MS (ESI+, m/e) 545 (M+1)

### Example 583

### 4-{[(3R)-3-Benzyl-4-({1-[(1R,2S)-2-hydroxy-2-(methoxymethyl)cyclohexyl]-5-phenyl-1H-imidazol-4-yl}carbonyl)piperazin-1-yl]methyl}-5-methyl-1,3-dioxol-2-one

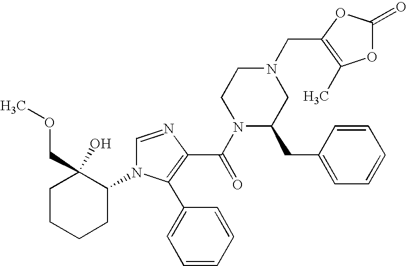

(1S,2R)-2-(4-{[(2R)-2-Benzylpiperazin-1-yl]carbonyl}-5-phenyl-1H-imidazol-1-yl)-1-(methoxymethyl)cyclohexanol obtained in the course of Example 367 (489 mg) and 4-(chloromethyl)-5-methyl-1,3-dioxol-2-one (149 mg) were dissolved in DMF (5 ml), potassium hydrogen carbonate (150 mg) was added, and the mixture was stirred at room temperature for 15 hr. To the reaction mixture was added saturated aqueous sodium hydrogen carbonate, and the mixture was extracted with ethyl acetate. The extract was washed with saturated brine, dried over anhydrous magnesium sulfate, and concentrated under reduced pressure. The residue was subjected to basic silica gel column chromatography, and the fraction eluted with ethyl acetate-methanol (7:3) was concentrated under reduced pressure to give the object compound (28 mg) as an amorphous solid.

MS (ESI+, m/e) 601 (M+1)

### Example 584

### 1-[4-(2-{(2R)-1-({1-[(1R,2S)-2-Hydroxy-2-(methoxymethyl)cyclohexyl]-5-phenyl-1H-imidazol-4-yl}carbonyl)-4-[(5-methyl-2-oxo-1,3-dioxol-4-yl)methyl]piperazin-2-yl}ethoxy)phenyl]pyrrolidin-2-one

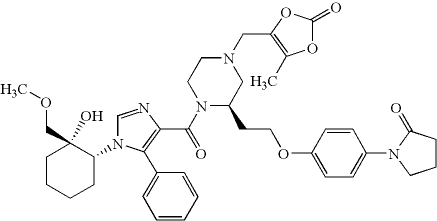

1-(4-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}phenyl)pyrrolidin-2-one (the compound of Example 294) (105 mg) and potassium hydrogen carbonate were suspended in DMF (3 ml). A solution of 4-(bromomethyl)-5-methyl-1,3-dioxol-2-one (37 mg) in DMF (2 ml) which was cooled to 0° C. was added dropwise thereto, and the mixture was stirred at 0° C. for 1 hr, and then at room temperature for 3 hr. To the reaction mixture was added saturated aqueous sodium hydrogen carbonate, and the mixture was extracted with ethyl acetate. The extract was washed with saturated brine, dried over anhydrous magnesium sulfate, and concentrated under reduced pressure. The residue was subjected to basic silica gel column chromatography, and the fraction eluted with ethyl acetate-methanol (7:3) was concentrated under reduced pressure to give the object compound (71 mg) as an amorphous solid.

MS (ESI+, m/e) 714 (M+1)

In the same manner as in Example 1 (Method A) except that the treatment of the final product with 4N hydrogen chloride-ethyl acetate solution was omitted, the following compounds (Examples 585-588) were obtained as a free amorphous solid.

### Example 585

### (1S,2R)-2-{4-[((2R)-2-{2-[(4-Methoxy-2-methylphenyl)amino]ethyl}piperazin-1-yl)carbonyl]-5-phenyl-1H-imidazol-1-yl}-1-methylcyclohexanol

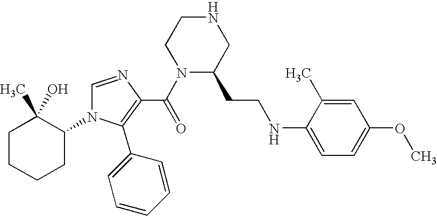

MS (ESI+, m/e) 532 (M+1)

### Example 586

### (1S,2R)-2-{4-[((2R)-2-{2-[(5-Methoxy-2-methylphenyl)amino]ethyl}piperazin-1-yl)carbonyl]-5-phenyl-1H-imidazol-1-yl}-1-methylcyclohexanol

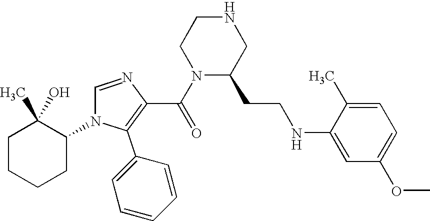

MS (ESI+, m/e) 532 (M+1)

### Example 587

### Methyl ((1S,2S)-2-{4-[((2R)-2-{2-[(4-methoxy-2-methylphenyl)amino]ethyl}piperazin-1-yl)carbonyl]-5-phenyl-1H-imidazol-1-yl}cyclohexyl)carbamate

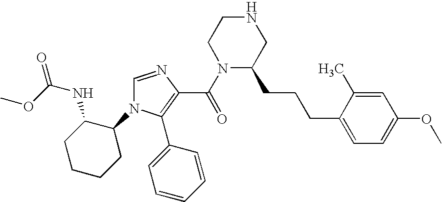

MS (ESI+, m/e) 575 (M+1)

### Example 588

### Methyl ((1S,2S)-2-{4-[((2R)-2-{2-[(5-methoxy-2-methylphenyl)amino]ethyl}piperazin-1-yl)carbonyl]-5-phenyl-1H-imidazol-1-yl}cyclohexyl)carbamate

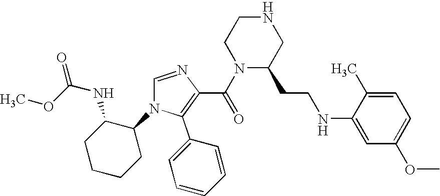

MS (ESI+, m/e) 575 (M+1)

In the same manner as in Example 9 (Method I) except that the treatment of the final product (excluding Example 595) with 4N hydrogen chloride-ethyl acetate solution was omitted, the following compounds (Examples 589-594) were obtained as a free amorphous solid.

### Example 589

### (1S,2R)-2-[4-({(2R)-2-[2-(2,3-Dihydro-1-benzofuran-6-yloxy)ethyl]piperazin-1-yl}carbonyl)-5-phenyl-1H-imidazol-1-yl]-1-(methoxymethyl)cyclohexanol

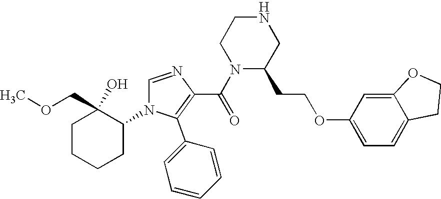

MS (ESI+, m/e) 561 (M+1)

### Example 590

### (1S,2R)-2-{4-[((2R)-2-{2-[(3-Fluoro-2-methoxyphenyl)amino]ethyl}piperazin-1-yl)carbonyl]-5-phenyl-1H-imidazol-1-yl}-1-methylcyclohexanol

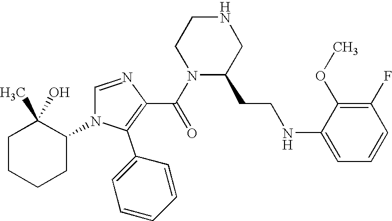

MS (ESI+, m/e) 536 (M+1)

### Example 591

### Methyl ((1S,2S)-2-{4-[((2R)-2-{2-[(3-fluoro-2-methoxyphenyl)amino]ethyl}piperazin-1-yl)carbonyl]-5-phenyl-1H-imidazol-1-yl}cyclohexyl)carbamate

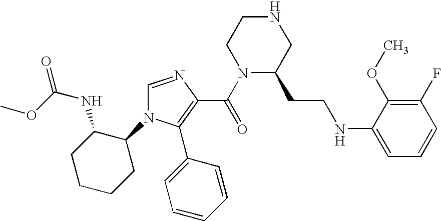

MS (ESI+, m/e) 579 (M+1)

### Example 592

### (1S,2R)-1-(Methoxymethyl)-2-{4-[((2R)-2-{2-[(2-methyl-1,3-benzoxazol-5-yl)oxy]ethyl}piperazin-1-yl)carbonyl]-5-phenyl-1H-imidazol-1-yl}cyclohexanol

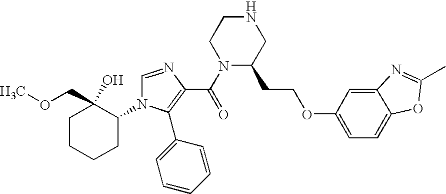

MS (ESI+, m/e) 574 (M+1)

### Example 593

### (1R,2R)-1-(Cyclopropylmethyl)-2-{4-[((2R)-2-{2-[(2-methyl-1,3-benzoxazol-5-yl)oxy]ethyl}piperazin-1-yl)carbonyl]-5-phenyl-1H-imidazol-1-yl}cyclohexanol

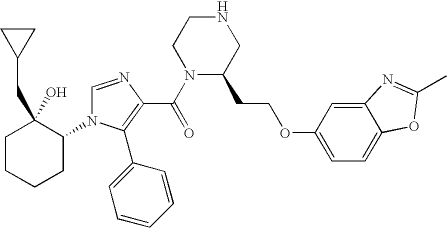

MS (ESI+, m/e) 584 (M+1)

### Example 594

### 1-({4-[((2R)-2-{2-[(3-Fluoro-2-methoxyphenyl)amino]ethyl}piperazin-1-yl)carbonyl]-5-phenyl-1H-imidazol-1-yl}methyl)cyclohexanol

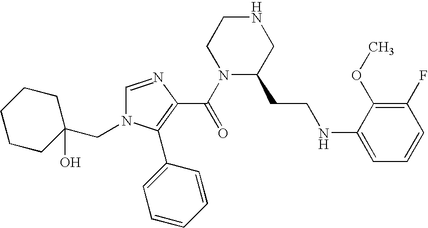

MS (ESI+, m/e) 536 (M+1)

In the same manner as in Example 8 (Method H), the following compound (Example 595) was obtained.

### Example 595

### (1R,2R)-1-(Cyclopropylmethyl)-2-[5-phenyl-4-({(2S)-2-[(phenylsulfonyl)methyl]piperazin-1-yl}carbonyl)-1H-imidazol-1-yl]cyclohexanol dihydrochloride

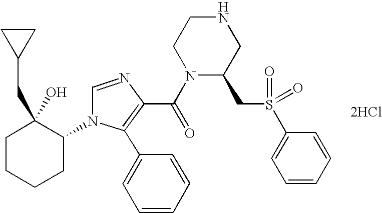

MS (ESI+, m/e) 563 (M+1)

In the same manner as in Example 10 (Method J) except that the treatment of the final product with 4N hydrogen chloride-ethyl acetate solution was omitted, the following compound (Examples 596) was obtained as a free amorphous solid.

### Example 596

### [2-({2-[(2R)-1-({1-[(1R,2S)-2-Hydroxy-2-(methoxymethyl)cyclohexyl]-5-phenyl-1H-imidazol-4-yl}carbonyl)piperazin-2-yl]ethyl}thio)-1,3-thiazol-4-yl]methyl acetate and (1S,2R)-2-(4-{[(2R)-2-(2-{[4-(hydroxymethyl)-1,3-thiazol-2-yl]thio}ethyl)piperazin-1-yl]carbonyl}-5-phenyl-1H-imidazol-1-yl)-1-(methoxymethyl)cyclohexanol

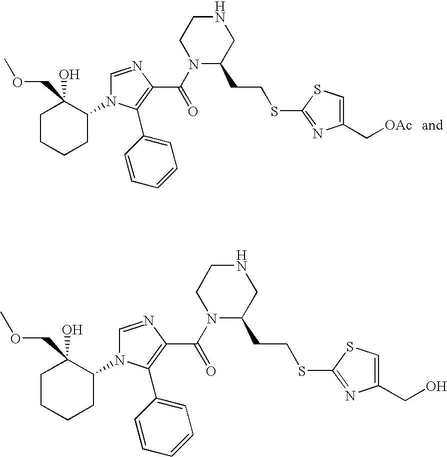

After the reaction by Method J, the residue was subjected to basic silica gel column chromatography, and the fraction eluted with ethyl acetate-methanol (100:0-80:20) was concentrated under reduced pressure to give [2-({2-[(2R)-1-({1-[(1R,2S)-2-hydroxy-2-(methoxymethyl)cyclohexyl]-5-phenyl-1H-imidazol-4-yl}carbonyl)piperazin-2-yl]ethyl}thio)-1,3-thiazol-4-yl]methyl acetate (113 mg, MS (ESI+, m/e) 614 (M+1)) as a component having a short retention time, and (1S,2R)-2-(4-{[(2R)-2-(2-{[4-(hydroxymethyl)-1,3-thiazol-2-yl]thio}ethyl)piperazin-1-yl]carbonyl}-5-phenyl-1H-imidazol-1-yl)-1-(methoxymethyl)cyclohexanol (20 mg, MS (ESI+, m/e) 570 (M+1)) as a component having a long retention time.

The following compounds of Examples 597-644 can be synthesized according to the above-mentioned methods.

### Example 597

### (1S,2R)-2-{4-[((2R)-2-{2-[(2,4-Dimethyl-1,3-benzoxazol-5-yl)oxy]ethyl}piperazin-1-yl)carbonyl]-5-phenyl-1H-imidazol-1-yl}-1-(methoxymethyl)cyclohexanol

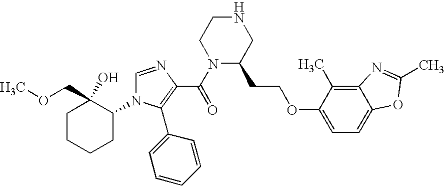

### Example 598

### (1S,2R)-2-{4-[((2R)-2-{2-[(4-Fluoro-2-methyl-1,3-benzoxazol-5-yl)oxy]ethyl}piperazin-1-yl)carbonyl]-5-phenyl-1H-imidazol-1-yl}-1-(methoxymethyl)cyclohexanol

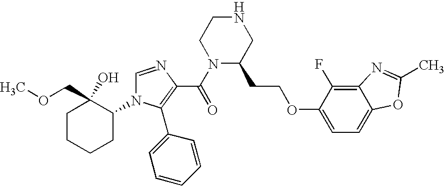

### Example 599

### (1S,2R)-2-{4-[((2R)-2-{2-[(7-Fluoro-2-methyl-1,3-benzoxazol-6-yl)oxy]ethyl}piperazin-1-yl)carbonyl]-5-phenyl-1H-imidazol-1-yl}-1-(methoxymethyl)cyclohexanol

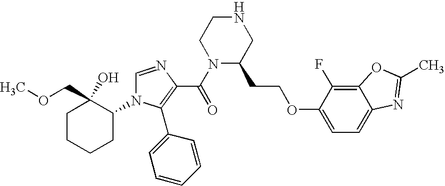

### Example 600

### (1S,2R)-2-{4-[((2R)-2-{2-[(2,7-Dimethyl-1,3-benzoxazol-6-yl)oxy]ethyl}piperazin-1-yl)carbonyl]-5-phenyl-1H-imidazol-1-yl}-1-(methoxymethyl)cyclohexanol

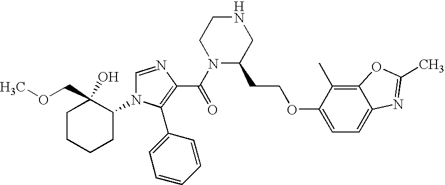

### Example 601

### (1S,2R)-2-{4-[((2R)-2-{2-[(2,6-Dimethyl-1,3-benzoxazol-5-yl)oxy]ethyl}piperazin-1-yl)carbonyl]-5-phenyl-1H-imidazol-1-yl}-1-(methoxymethyl)cyclohexanol

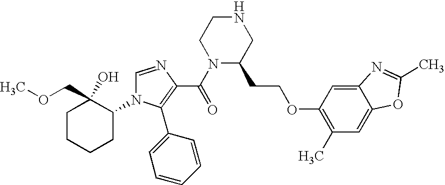

### Example 602

### (1S,2R)-2-{4-[((2R)-2-{2-[(6-Fluoro-2-methyl-1,3-benzoxazol-5-yl)oxy]ethyl}piperazin-1-yl)carbonyl]-5-phenyl-1H-imidazol-1-yl}-1-(methoxymethyl)cyclohexanol

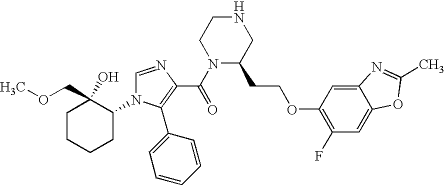

### Example 603

### (1S,2R)-2-{4-[((2R)-2-{2-[(2,7-Dimethyl-1,3-benzoxazol-5-yl)oxy]ethyl}piperazin-1-yl)carbonyl]-5-phenyl-1H-imidazol-1-yl}-1-(methoxymethyl)cyclohexanol

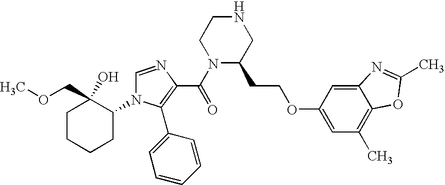

### Example 604

### (1S,2R)-2-{4-[((2R)-2-{2-[(7-Fluoro-2-methyl-1,3-benzoxazol-5-yl)oxy]ethyl}piperazin-1-yl)carbonyl]-5-phenyl-1H-imidazol-1-yl}-1-(methoxymethyl)cyclohexanol

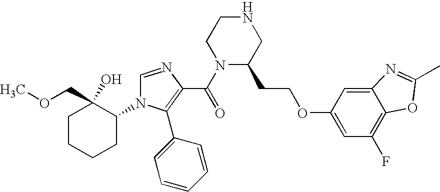

### Example 605

### (1S,2R)-2-{4-[((2R)-2-{2-[(2-Cyclopropyl-1,3-benzoxazol-5-yl)oxy]ethyl}piperazin-1-yl)carbonyl]-5-phenyl-1H-imidazol-1-yl}-1-(methoxymethyl)cyclohexanol

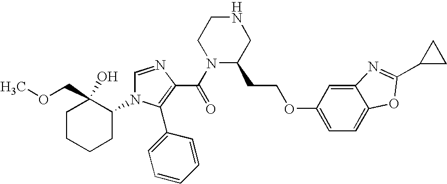

### Example 606

### (1S,2R)-2-{4-[((2R)-2-{2-[(2-Ethyl-1,3-benzoxazol-5-yl)oxy]ethyl}piperazin-1-yl)carbonyl]-5-phenyl-1H-imidazol-1-yl}-1-(methoxymethyl)cyclohexanol

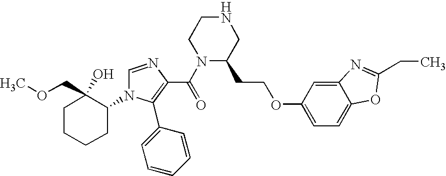

### Example 607

### (1S,2R)-2-{4-[((2R)-2-{2-[(2-Cyclopropyl-4-methyl-1,3-benzoxazol-5-yl)oxy]ethyl}piperazin-1-yl)carbonyl]-5-phenyl-1H-imidazol-1-yl}-1-(methoxymethyl)cyclohexanol

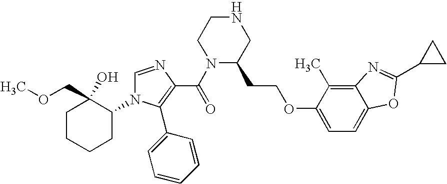

### Example 608

### (1S,2R)-2-{4-[((2R)-2-{2-[(2-Cyclopropyl-4-fluoro-1,3-benzoxazol-5-yl)oxy]ethyl}piperazin-1-yl)carbonyl]-5-phenyl-1H-imidazol-1-yl}-1-(methoxymethyl)cyclohexanol

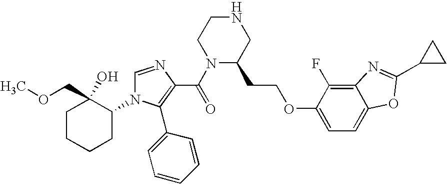

### Example 609

### (1S,2R)-2-[4-({(2R)-2-[2-(1,2-Benzisoxazol-5-yloxy)ethyl]piperazin-1-yl}carbonyl)-5-phenyl-1H-imidazol-1-yl]-1-(methoxymethyl)cyclohexanol

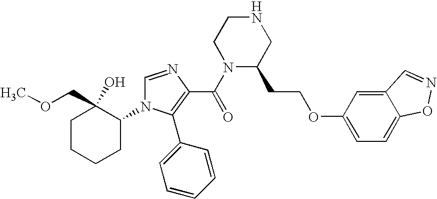

### Example 610

### (1S,2R)-2-[4-({(2R)-2-[2-(1,2-Benzisoxazol-6-yloxy)ethyl]piperazin-1-yl}carbonyl)-5-phenyl-1H-imidazol-1-yl]-1-(methoxymethyl)cyclohexanol

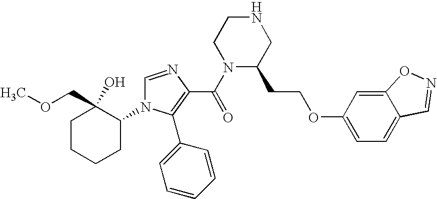

### Example 611

### (1S,2R)-1-(Methoxymethyl)-2-{4-[((2R)-2-{2-[(2-methylimidazo[1,2-a]pyridin-6-yl)oxy]ethyl}piperazin-1-yl)carbonyl]-5-phenyl-1H-imidazol-1-yl}cyclohexanol

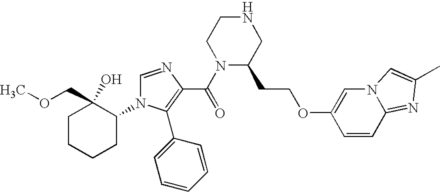

### Example 612

### (1S,2R)-1-(Methoxymethyl)-2-{4-[((2R)-2-{2-[(2-methylimidazo[1,2-a]pyridine-7-yl)oxy]ethyl}piperazin-1-yl)carbonyl]-5-phenyl-1H-imidazol-1-yl}cyclohexanol

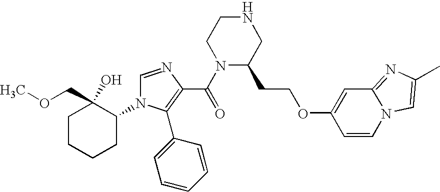

### Example 613

### (1S,2R)-2-[4-({(2R)-2-[2-(1,2-Benzisothiazol-5-yloxy)ethyl]piperazin-1-yl}carbonyl)-5-phenyl-1H-imidazol-1-yl]-1-(methoxymethyl)cyclohexanol

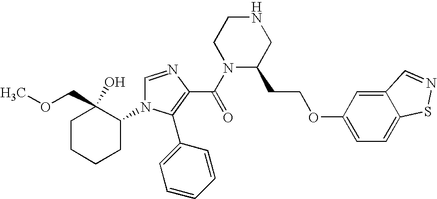

### Example 614

### (1S,2R)-2-[4-({(2R)-2-[2-(1,2-Benzisothiazol-6-yloxy)ethyl]piperazin-1-yl}carbonyl)-5-phenyl-1H-imidazol-1-yl]-1-(methoxymethyl)cyclohexanol

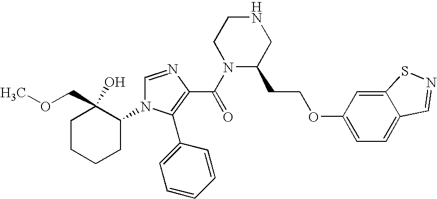

### Example 615

### (1S,2R)-2-{4-[((2R)-2-{2-[(1,2-Dimethyl-1H-indol-5-yl)oxy]ethyl}piperazin-1-yl)carbonyl]-5-phenyl-1H-imidazol-1-yl}-1-(methoxymethyl)cyclohexanol

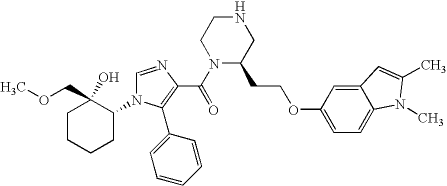

### Example 616

### (1S,2R)-2-{4-[((2R)-2-{2-[(1,2-Dimethyl-1H-indol-6-yl)oxy]ethyl}piperazin-1-yl)carbonyl]-5-phenyl-1H-imidazol-1-yl}-1-(methoxymethyl)cyclohexanol

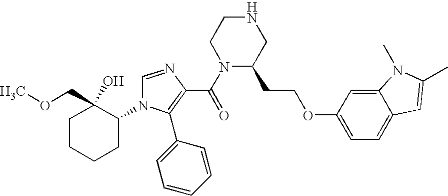

### Example 617

### (1S,2R)-1-(Methoxymethyl)-2-{4-[((2R)-2-{2-[(2-methyl-1-benzofuran-5-yl)oxy]ethyl}piperazin-1-yl)carbonyl]-5-phenyl-1H-imidazol-1-yl}cyclohexanol

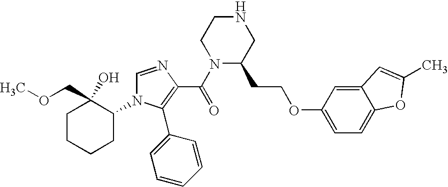

### Example 618

### (1S,2R)-1-(Methoxymethyl)-2-{4-[((2R)-2-{2-[(2-methyl-1-benzofuran-6-yl)oxy]ethyl}piperazin-1-yl)carbonyl]-5-phenyl-1H-imidazol-1-yl}cyclohexanol

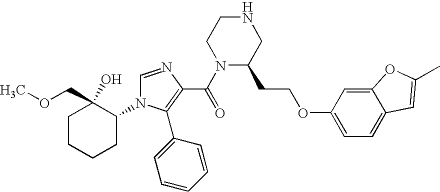

### Example 619

### (1S,2R)-2-{4-[((2R)-2-{2-[(2,4-Dimethyl-1,3-benzoxazol-5-yl)oxy]ethyl}piperazin-1-yl)carbonyl]-5-phenyl-1H-imidazol-1-yl}-1-methylcyclohexanol

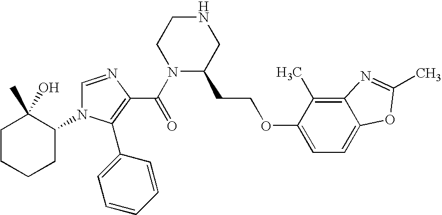

### Example 620

### (1S,2R)-2-{4-[((2R)-2-{2-[(4-Fluoro-2-methyl-1,3-benzoxazol-5-yl)oxy]ethyl}piperazin-1-yl)carbonyl]-5-phenyl-1H-imidazol-1-yl}-1-methylcyclohexanol

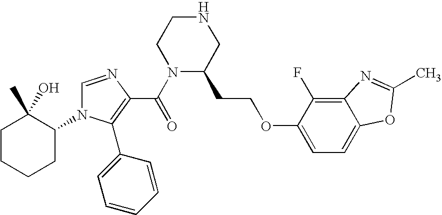

### Example 621

### (1S,2R)-2-{4-[((2R)-2-{2-[(2-Cyclopropyl-1,3-benzoxazol-5-yl)oxy]ethyl}piperazin-1-yl)carbonyl]-5-phenyl-1H-imidazol-1-yl}-1-methylcyclohexanol

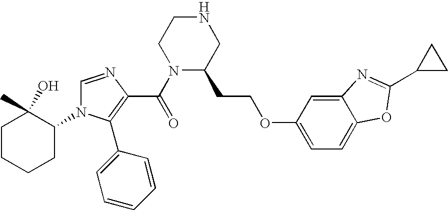

### Example 622

### (1R,2R)-2-{4-[((2R)-2-{2-[(2-Cyclopropyl-1,3-benzoxazol-5-yl)oxy]ethyl}piperazin-1-yl)carbonyl]-5-phenyl-1H-imidazol-1-yl}-1-(cyclopropylmethyl)cyclohexanol

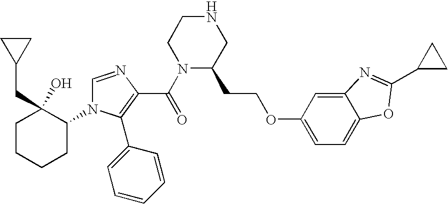

### Example 623

### (1S,2R)-2-{4-[((2R)-2-{2-[(7-Fluoro-2-methyl-1,3-benzoxazol-6-yl)oxy]ethyl}piperazin-1-yl)carbonyl]-5-phenyl-1H-imidazol-1-yl}-1-methylcyclohexanol

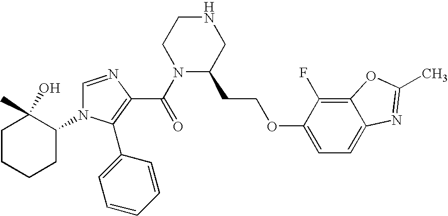

### Example 624

### (1S,2R)-2-{4-[((2R)-2-{2-[(2,7-Dimethyl-1,3-benzoxazol-6-yl)oxy]ethyl}piperazin-1-yl)carbonyl]-5-phenyl-1H-imidazol-1-yl}-1-methylcyclohexanol

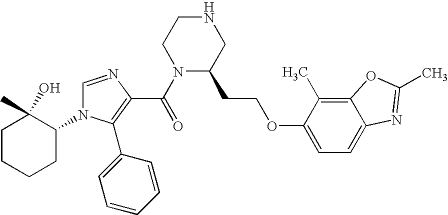

### Example 625

### (1R,2R)-1-(Cyclopropylmethyl)-2-{4-[((2R)-2-{2-[(2,4-dimethyl-1,3-benzoxazol-5-yl)oxy]ethyl}piperazin-1-yl)carbonyl]-5-phenyl-1H-imidazol-1-yl}cyclohexanol

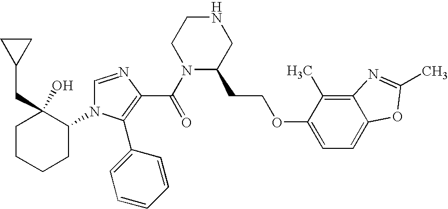

### Example 626

### (1R,2R)-1-(Cyclopropylmethyl)-2-{4-[((2R)-2-{2-[(4-fluoro-2-methyl-1,3-benzoxazol-5-yl)oxy]ethyl}piperazin-1-yl)carbonyl]-5-phenyl-1H-imidazol-1-yl}cyclohexanol

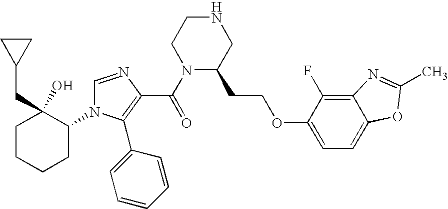

### Example 627

### Ethyl {(1S,2S)-2-[4-({(2R)-2-[2-(3,5-difluorophenoxy)ethyl]piperazin-1-yl}carbonyl)-5-phenyl-1H-imidazol-1-yl]cyclohexyl}carbamate

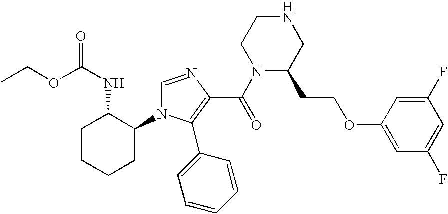

### Example 628

### Methyl {(1S,2S)-2-[4-({(2R)-2-[2-(3,5-dichlorophenoxy)ethyl]piperazin-1-yl}carbonyl)-5-phenyl-1H-imidazol-1-yl]cyclohexyl}carbamate

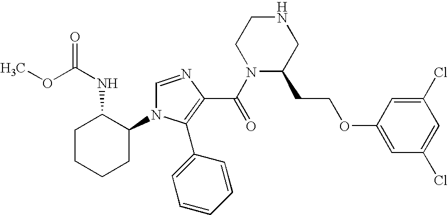

### Example 629

### Methyl {(1S,2S)-2-[4-({(2R)-2-[2-(3-chloro-5-fluorophenoxy)ethyl]piperazin-1-yl}carbonyl)-5-phenyl-1H-imidazol-1-yl]cyclohexyl}carbamate

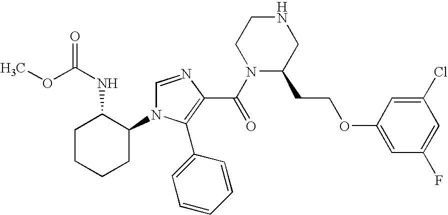

### Example 630

### Methyl {(1S,2S)-2-[4-({(2R)-2-[2-(3-fluoro-4-methylphenoxy)ethyl]piperazin-1-yl}carbonyl)-5-phenyl-1H-imidazol-1-yl]cyclohexyl}carbamate

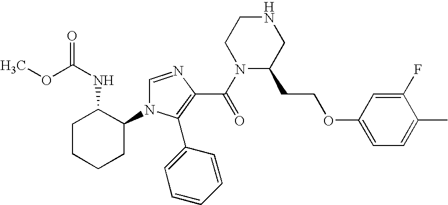

### Example 631

### Methyl {(1S,2S)-2-[4-({(2R)-2-[2-(3-chloro-4-methylphenoxy)ethyl]piperazin-1-yl}carbonyl)-5-phenyl-1H-imidazol-1-yl]cyclohexyl}carbamate

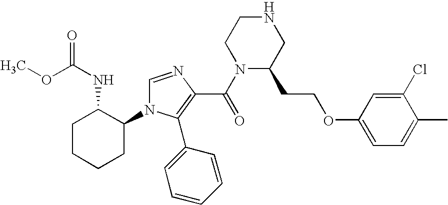

### Example 632

### Methyl ((1S,2S)-2-{4-[((2R)-2-{2-[(2,2-dimethyl-2,3-dihydro-1-benzofuran-6-yl)oxy]ethyl}piperazin-1-yl)carbonyl]-5-phenyl-1H-imidazol-1-yl}cyclohexyl)carbamate

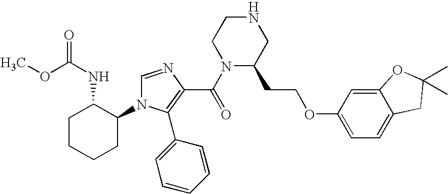

### Example 633

### Methyl {(1S,2S)-2-[4-({(2R)-2-[2-(2-naphthyloxy)ethyl]piperazin-1-yl}carbonyl)-5-phenyl-1H-imidazol-1-yl]cyclohexyl}carbamate

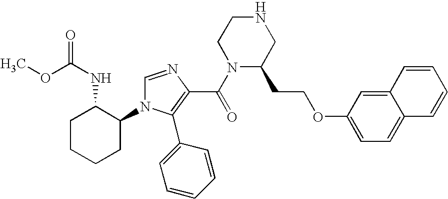

### Example 634

### Methyl {(1S,2S)-2-[4-({(2R)-2-[2-(3,4-difluorophenoxy)ethyl]piperazin-1-yl}carbonyl)-5-phenyl-1H-imidazol-1-yl]cyclohexyl}carbamate

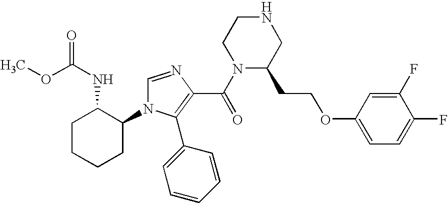

### Example 635

### Methyl {(1S,2S)-2-[4-({(2R)-2-[2-(3,4-dichlorophenoxy)ethyl]piperazin-1-yl}carbonyl)-5-phenyl-1H-imidazol-1-yl]cyclohexyl}carbamate

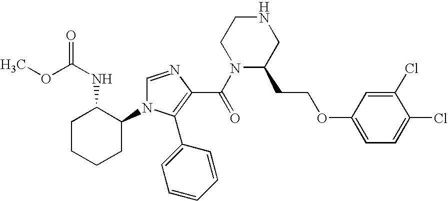

### Example 636

### (1S,2R)-2-{4-[((2R)-2-{2-[(2-Methoxyphenyl)amino]ethyl}piperazin-1-yl)carbonyl]-5-phenyl-1H-imidazol-1-yl}-1-methylcyclohexanol

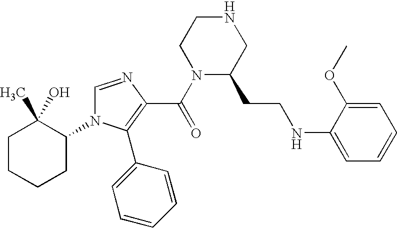

### Example 637

### (1S,2R)-2-{4-[((2R)-2-{2-[(2-Methoxy-5-methylphenyl)amino]ethyl}piperazin-1-yl)carbonyl]-5-phenyl-1H-imidazol-1-yl}-1-methylcyclohexanol

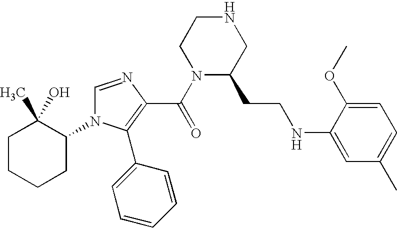

### Example 638

### (1S,2R)-1-Methyl-2-{4-[((2R)-2-{2-[(2-methylphenyl)amino]ethyl}piperazin-1-yl)carbonyl]-5-phenyl-1H-imidazol-1-yl}cyclohexanol

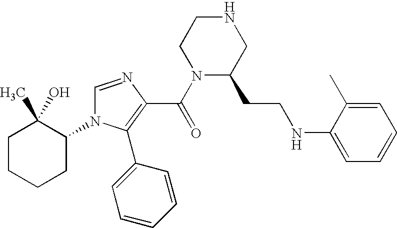

### Example 639

### (1S,2R)-2-{4-[((2R)-2-{2-[(2-Fluorophenyl)amino]ethyl}piperazin-1-yl)carbonyl]-5-phenyl-1H-imidazol-1-yl}-1-methylcyclohexanol

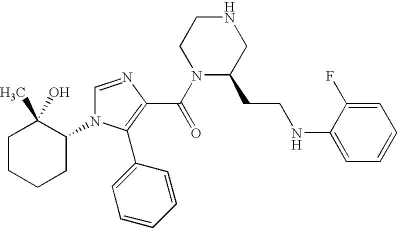

### Example 640

### (1S,2R)-2-{4-[((2R)-2-{2-[(2-Methoxy-4-methylphenyl)amino]ethyl}piperazin-1-yl)carbonyl]-5-phenyl-1H-imidazol-1-yl}-1-methylcyclohexanol

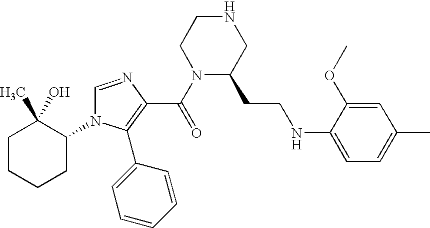

### Example 641

### (1S,2R)-2-{4-[((2R)-2-{2-[(4-Methoxyphenyl)amino]ethyl}piperazin-1-yl)carbonyl]-5-phenyl-1H-imidazol-1-yl}-1-methylcyclohexanol

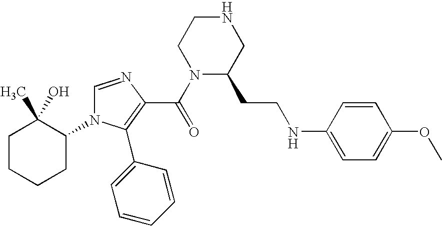

### Example 642

### (1S,2R)-2-{4-[((2R)-2-{2-[(3-Fluoro-4-methoxyphenyl)amino]ethyl}piperazin-1-yl)carbonyl]-5-phenyl-1H-imidazol-1-yl}-1-methylcyclohexanol

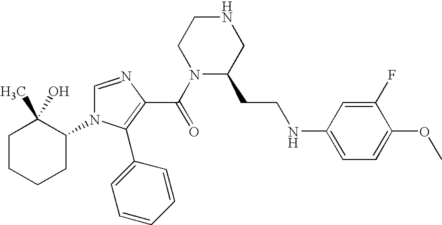

### Example 643

### (1S,2R)-2-{4-[((2R)-2-{2-[(3-Methoxyphenyl)amino]ethyl}piperazin-1-yl)carbonyl]-5-phenyl-1H-imidazol-1-yl}-1-methylcyclohexanol

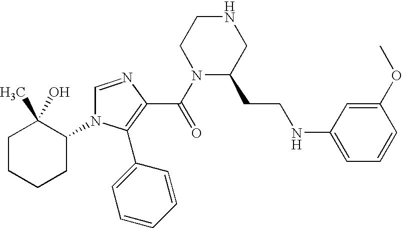

### Example 644

### (1S,2R)-2-{4-[((2R)-2-{2-[(4-Fluoro-2-methoxyphenyl)amino]ethyl}piperazin-1-yl)carbonyl]-5-phenyl-1H-imidazol-1-yl}-1-methylcyclohexanol

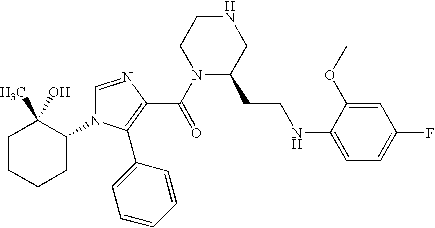

### Example 645

### (1S,2R)-2-(4-{[(2R)-2-Benzylpiperazin-1-yl]carbonyl}-5-phenyl-1H-imidazol-1-yl)-1-(methoxymethyl)cyclohexanol 0.5 fumarate

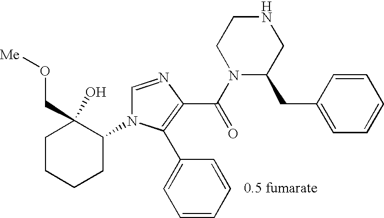

(1S,2R)-2-(4-{[(2R)-2-Benzylpiperazin-1-yl]carbonyl}-5-phenyl-1H-imidazol-1-yl)-1-(methoxymethyl)cyclohexanol fumarate (50 mg) was dissolved in methanol (1.5 ml) at 60° C., ethyl acetate (15 ml) was added, and the mixture was cooled to 0° C. The precipitated crystals were collected by filtration to give the object compound (41 mg).

### Example 646

### (1S,2R)-1-(Methoxymethyl)-2-(4-{[(2R)-2-{2-[(5-methoxy-2-methylphenyl)amino]ethyl}piperazin-1-yl]carbonyl}-5-phenyl-1H-imidazol-1-yl)cyclohexanol fumarate

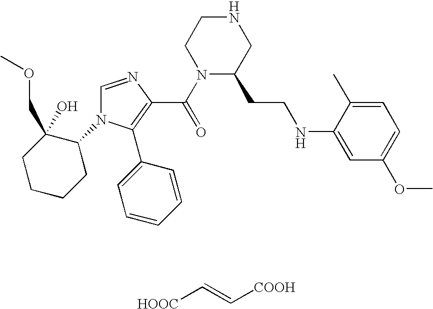

(1S,2R)-1-(Methoxymethyl)-2-(4-{[(2R)-2-{2-[(5-methoxy-2-methylphenyl)amino]ethyl}piperazin-1-yl]carbonyl}-5-phenyl-1H-imidazol-1-yl)cyclohexanol (3.75 g) and fumaric acid (736 mg) were dissolved in ethanol (100 ml) while heating (60° C.), and the solvent (about 50 ml) was evaporated under reduced pressure. To the residue was added acetonitrile (150 ml), and the solvent (about 100 ml) was evaporated under reduced pressure. The residue was left to stand at room temperature for 1 hr, and the crystals were collected by filtration, washed with a small amount of acetonitrile, and dried under reduced pressure to give the object compound (3.5 g) as crystals.

melting point: 157-158° C.

### Example 647

### Methyl [(1S,2S)-2-(4-{[(2R)-2-(3,5-difluorobenzyl)piperazin-1-yl]carbonyl}-5-phenyl-1H-imidazol-1-yl)cyclohexyl]carbamate succinate

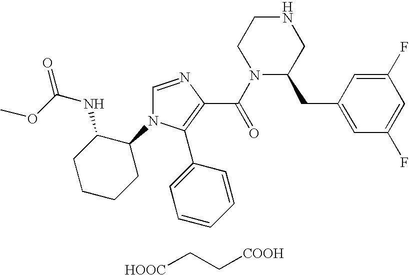

Methyl [(1S,2S)-2-(4-{[(2R)-2-(3,5-difluorobenzyl)piperazin-1-yl]carbonyl}-5-phenyl-1H-imidazol-1-yl)cyclohexyl]carbamate (900 mg) and succinic acid (197 mg) were dissolved in ethanol (20 ml) while heating (60° C.), and the solvent was evaporated under reduced pressure. To the residue were added acetonitrile (20 ml) and ethyl acetate (30 ml), and the mixture was stirred at room temperature for 12 hr. The crystals were collected by filtration, washed with a small amount of acetonitrile, and dried under reduced pressure to give the object compound (800 mg) as crystals.

melting point: 157-176° C.

### Example 648

### Ethyl [(1S,2S)-2-(4-{[(2R)-2-(3,5-difluorobenzyl)piperazin-1-yl]carbonyl}-5-phenyl-1H-imidazol-1-yl)cyclohexyl]carbamate malonate

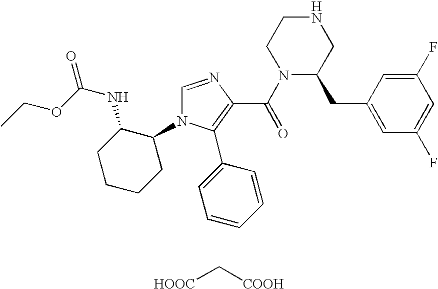

Ethyl [(1S,2S)-2-(4-{[(2R)-2-(3,5-difluorobenzyl)piperazin-1-yl]carbonyl}-5-phenyl-1H-imidazol-1-yl)cyclohexyl]carbamate (36 g) and malonic acid (6.45 g) were dissolved in ethanol (500 ml) while heating (80° C.), and the solvent was evaporated under reduced pressure. To the residue were added ethanol (300 ml) and water (30 ml), and the mixture was heated (80° C.). Ethyl acetate (300 ml) was added, and the mixture was stirred at room temperature for 12 hr. The crystals were collected by filtration, washed with a small amount of ethyl acetate, and dried under reduced pressure to give the object compound as crystals (25.2 g).

melting point: 166-167° C.

### Example 649

### Propyl [(1S,2S)-2-(4-{[(2R)-2-(3,5-difluorobenzyl)piperazin-1-yl]carbonyl}-5-phenyl-1H-imidazol-1-yl)cyclohexyl]carbamate

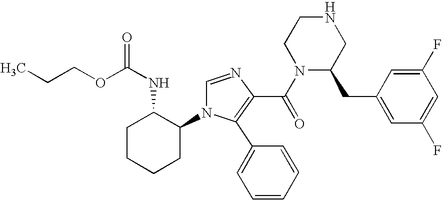

(1S,2S)-2-(4-{[(2R)-4-Benzyl-2-(3,5-difluorobenzyl)piperazin-1-yl]carbonyl}-5-phenyl-1H-imidazol-1-yl)cyclohexanamine (171 mg) and DMAP (44 mg) were dissolved in THF (3 ml), propyl chlorocarbonate (39 mg) was added, and the mixture was stirred at room temperature for 15 hr. To the reaction mixture was added aqueous sodium bicarbonate, and the mixture was extracted with ethyl acetate. The extract was washed with saturated brine, dried over anhydrous magnesium sulfate, and concentrated. The residue was subjected to basic silica gel column chromatography, and the fraction eluted with ethyl acetate-methanol (9:1) was concentrated under reduced pressure to give propyl [(1S,2S)-2-(4-{[(2R)-4-benzyl-2-(3,5-difluorobenzyl)piperazin-1-yl]carbonyl}-5-phenyl-1H-imidazol-1-yl)cyclohexyl]carbamate (182 mg). The obtained propyl [(1S,2S)-2-(4-{[(2R)-4-benzyl-2-(3,5-difluorobenzyl)piperazin-1-yl]carbonyl}-5-phenyl-1H-imidazol-1-yl)cyclohexyl]carbamate (182 mg) was dissolved in methanol (5 ml), 20% palladium hydroxide-carbon (50% containing water) (20 mg) was added, and the mixture was subjected to catalytic reduction at ambient temperature and normal pressure for 15 hr. The catalyst was filtered off, and the filtrate was concentrated under reduced pressure to give the object compound (94 mg).

MS (ESI+, m/e) 566 (M+1)

In the same manner as in Example 649, the following compounds (Examples 650-654) were obtained.

### Example 650

### 3-[(1S,2S)-2-(4-{[(2R)-2-(3,5-Difluorobenzyl)piperazin-1-yl]carbonyl}-5-phenyl-1H-imidazol-1-yl)cyclohexyl]-1,1-dimethylurea

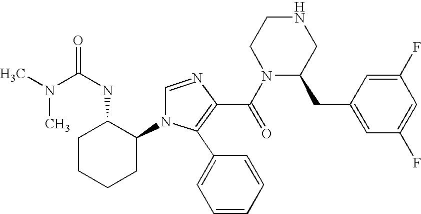

MS (ESI+, m/e) 551 (M+1)

### Example 651

### N-[(1S,2S)-2-(4-{[(2R)-2-(3,5-Difluorobenzyl)piperazin-1-yl]carbonyl}-5-phenyl-1H-imidazol-1-yl)cyclohexyl]propanamide

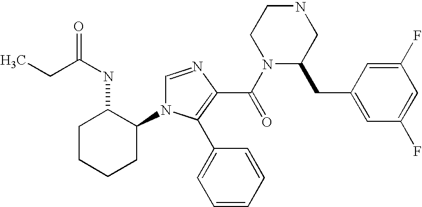

MS (ESI+, m/e) 536 (M+1)

### Example 652

### 2-Methoxyethyl [(1S,2S)-2-(4-{[(2R)-2-(3,5-difluorobenzyl)piperazin-1-yl]carbonyl}-5-phenyl-1H-imidazol-1-yl)cyclohexyl]carbamate

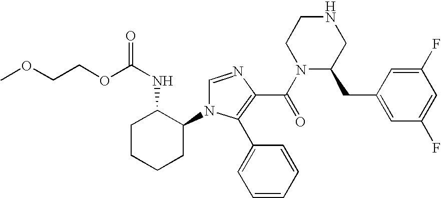

MS (ESI+, m/e) 582 (M+1)

### Example 653

### Isobutyl [(1S,2S)-2-(4-{[(2R)-2-(3,5-difluorobenzyl)piperazin-1-yl]carbonyl}-5-phenyl-1H-imidazol-1-yl)cyclohexyl]carbamate

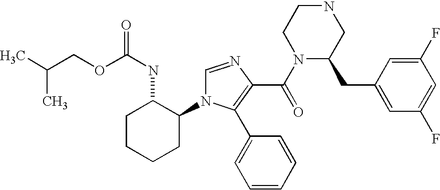

MS (ESI+, m/e) 580 (M+1)

### Example 654

### Isopropyl [(1S,2S)-2-(4-{[(2R)-2-(3,5-difluorobenzyl)piperazin-1-yl]carbonyl}-5-phenyl-1H-imidazol-1-yl)cyclohexyl]carbamate

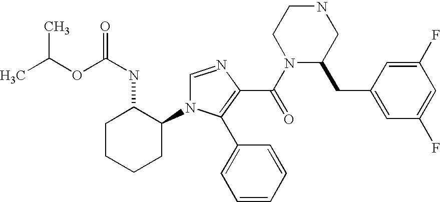

MS (ESI+, m/e) 566 (M+1)

### Example 655

### 2-Fluoroethyl [(1S,2S)-2-(4-{[(2R)-2-(3,5-difluorobenzyl)piperazin-1-yl]carbonyl}-5-phenyl-1H-imidazol-1-yl)cyclohexyl]carbamate

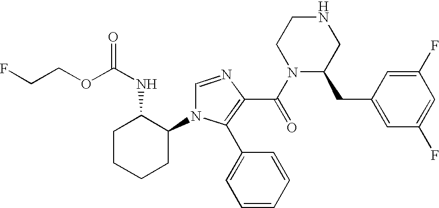

(1S,2S)-2-(4-{[(2R)-4-Benzyl-2-(3,5-difluorobenzyl)piperazin-1-yl]carbonyl}-5-phenyl-1H-imidazol-1-yl)cyclohexanamine (171 mg) and triethylamine (0.209 ml) were dissolved in THF (3 ml), 2-fluoroethyl chlorocarbonate (0.057 ml) was added, and the mixture was stirred at room temperature for 15 hr. To the reaction mixture was added aqueous sodium bicarbonate, and the mixture was extracted with ethyl acetate. The extract was washed with saturated brine, dried over anhydrous magnesium sulfate, and concentrated. The residue was subjected to basic silica gel column chromatography, and the fraction eluted with ethyl acetate-methanol (9:1) was concentrated under reduced pressure to give 2-fluoroethyl [(1S,2S)-2-(4-{[(2R)-4-benzyl-2-(3,5-difluorobenzyl)piperazin-1-yl]carbonyl}-5-phenyl-1H-imidazol-1-yl)cyclohexyl]carbamate (113 mg). The obtained 2-fluoroethyl [(1S,2S)-2-(4-{[(2R)-4-benzyl-2-(3,5-difluorobenzyl)piperazin-1-yl]carbonyl}-5-phenyl-1H-imidazol-1-yl)cyclohexyl]carbamate (113 mg) was dissolved in THF (5 ml), 20% palladium hydroxide-carbon (50% containing water) (20 mg) was added, and the mixture was subjected to catalytic reduction at ambient temperature and normal pressure for 15 hr. The catalyst was filtered off, and the filtrate was concentrated under reduced pressure to give the object compound (91 mg).

MS (ESI+, m/e) 570 (M+1)

In the same manner as in Example 655, the following compounds (Examples 656-659) were obtained.

### Example 656

### N-[(1S,2S)-2-(4-{[(2R)-2-(3,5-Difluorobenzyl)piperazin-1-yl]carbonyl}-5-phenyl-1H-imidazol-1-yl)cyclohexyl]methanesulfonamide

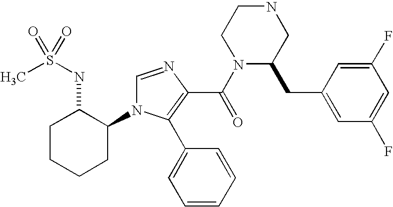

MS (ESI+, m/e) 558 (M+1)

### Example 657

### N′-[(1S,2S)-2-(4-{[(2R)-2-(3,5-Difluorobenzyl)piperazin-1-yl]carbonyl}-5-phenyl-1H-imidazol-1-yl)cyclohexyl]-N,N-dimethylsulfamide

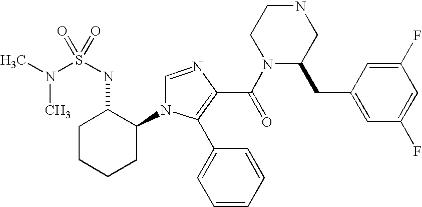

MS (ESI+, m/e) 587 (M+1)

### Example 658

### N-[(1S,2S)-2-(4-{[(2R)-2-(3,5-Difluorobenzyl)piperazin-1-yl]carbonyl}-5-phenyl-1H-imidazol-1-yl)cyclohexyl]propane-1-sulfonamide

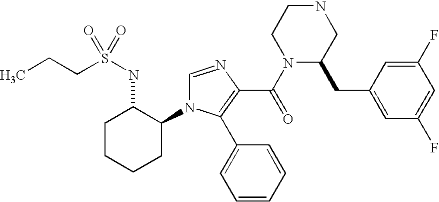

MS (ESI+, m/e) 586 (M+1)

### Example 659

### N-[(1S,2S)-2-(4-{[(2R)-2-(3,5-Difluorobenzyl)piperazin-1-yl]carbonyl}-5-phenyl-1H-imidazol-1-yl)cyclohexyl]ethanesulfonamide

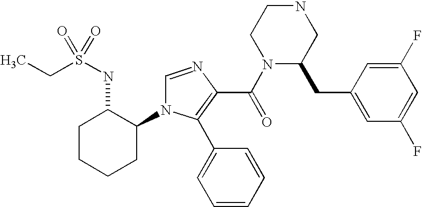

MS (ESI+, m/e) 572 (M+1)

### Example 660

### Cyclopropylmethyl [(1S,2S)-2-(4-{[(2R)-2-(3,5-Difluorobenzyl)piperazin-1-yl]carbonyl}-5-phenyl-1H-imidazol-1-yl)cyclohexyl]carbamate

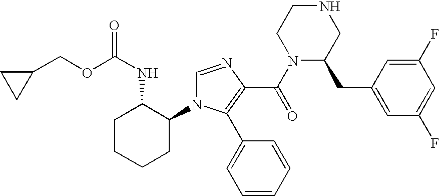

(1S,2S)-2-(4-{[(2R)-4-Benzyl-2-(3,5-difluorobenzyl)piperazin-1-yl]carbonyl}-5-phenyl-1H-imidazol-1-yl)cyclohexanamine (171 mg) and DMAP (110 mg) were dissolved in THF (5 ml), and the solution was ice-cooled. 4-Nitrophenyl chloroformate (91 mg) was added, and the mixture was stirred at 0° C. for 1 hr, and then at room temperature for 2 hr. To the reaction mixture was added cyclopropylmethanol (0.791 ml), and the mixture was stirred at 60° C. for 15 hr. The reaction mixture was poured into 1N aqueous sodium hydroxide solution, and the mixture was extracted with ethyl acetate. The extract was washed with saturated brine, dried over anhydrous magnesium sulfate, and concentrated under reduced pressure. The residue was subjected to basic silica gel column chromatography, and the fraction eluted with ethyl acetate-methanol (9:1) was concentrated under reduced pressure to give cyclopropylmethyl [(1S,2S)-2-(4-{[(2R)-4-benzyl-2-(3,5-difluorobenzyl)piperazin-1-yl]carbonyl}-5-phenyl-1H-imidazol-1-yl)cyclohexyl]carbamate (130 mg) as an amorphous solid. The obtained cyclopropylmethyl [(1S,2S)-2-(4-{[(2R)-4-benzyl-2-(3,5-difluorobenzyl)piperazin-1-yl]carbonyl}-5-phenyl-1H-imidazol-1-yl)cyclohexyl]carbamate (130 mg) was dissolved in THF (5 ml), 20% palladium hydroxide-carbon (50% containing water) (20 mg) was added, and the mixture was subjected to catalytic reduction at ambient temperature and normal pressure for 15 hr. The catalyst was filtered off, and the filtrate was concentrated under reduced pressure to give the object compound (88 mg).

MS (ESI+, m/e) 578 (M+1)

In the same manner as in Example 660, the following compounds (Examples 661-663) were obtained.

### Example 661

### 1-tert-Butyl-3-[(1S,2S)-2-(4-{[(2R)-2-(3,5-difluorobenzyl)piperazin-1-yl]carbonyl}-5-phenyl-1H-imidazol-1-yl)cyclohexyl]urea

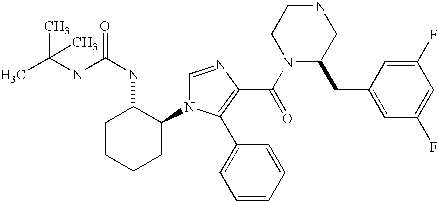

MS (ESI+, m/e) 579 (M+1)

### Example 662

### 2,2-Difluoroethyl [(1S,2S)-2-(4-{[(2R)-2-(3,5-difluorobenzyl)piperazin-1-yl]carbonyl}-5-phenyl-1H-imidazol-1-yl)cyclohexyl]carbamate

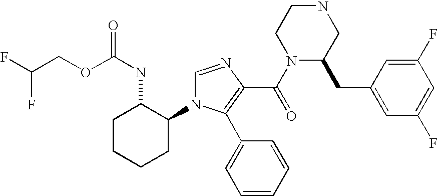

MS (ESI+, m/e) 588 (M+1)

### Example 663

### Cyclobutyl [(1S,2S)-2-(4-{[(2R)-2-(3,5-difluorobenzyl)piperazin-1-yl]carbonyl}-5-phenyl-1H-imidazol-1-yl)cyclohexyl]carbamate

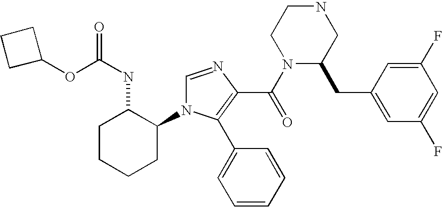

MS (ESI+, m/e) 578 (M+1)

In the same manner as in Example 1 (Method A) except that the treatment of the final compound with 4N hydrogen chloride-ethyl acetate solution was omitted, the following compounds (Examples 664-676) were obtained by isolating as a free amorphous solid.

### Example 664

### (1S,2R)-1-(Ethoxymethyl)-2-(4-{[(2R)-2-{2-[(4-methoxy-2-methylphenyl)amino]ethyl}piperazin-1-yl]carbonyl}-5-phenyl-1H-imidazol-1-yl)cyclohexanol

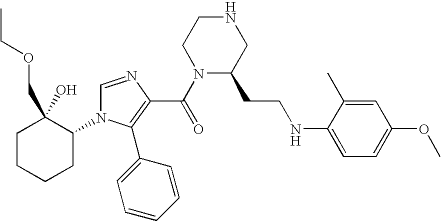

MS (ESI+, m/e) 576 (M+1)

### Example 665

### (1S,2R)-1-(Ethoxymethyl)-2-(4-{[(2R)-2-{2-[(5-methoxy-2-methylphenyl)amino]ethyl}piperazin-1-yl]carbonyl}-5-phenyl-1H-imidazol-1-yl)cyclohexanol

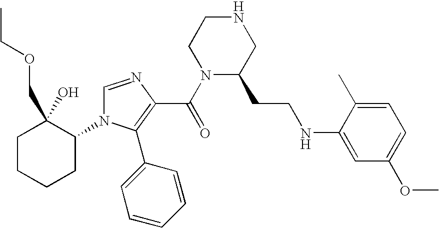

MS (ESI+, m/e) 576 (M+1)

### Example 666

### Cyclobutyl [(1S,2S)-2-(4-{[(2R)-2-{2-[(4-methoxy-2-methylphenyl)amino]ethyl}piperazin-1-yl]carbonyl}-5-phenyl-1H-imidazol-1-yl)cyclohexyl]carbamate

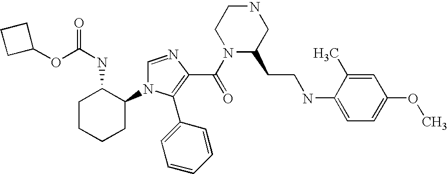

MS (ESI+, m/e) 615 (M+1)

### Example 667

### Cyclobutyl [(1S,2S)-2-(4-{[(2R)-2-{2-[(3-methoxy-2-methylphenyl)amino]ethyl}piperazin-1-yl]carbonyl}-5-phenyl-1H-imidazol-1-yl)cyclohexyl]carbamate

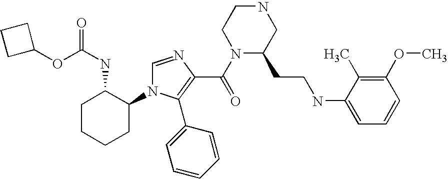

MS (ESI+, m/e) 615 (M+1)

### Example 668

### Isopropyl [(1S,2S)-2-(4-{[(2R)-2-{2-[(5-methoxy-2-methylphenyl)amino]ethyl}piperazin-1-yl]carbonyl}-5-phenyl-1H-imidazol-1-yl)cyclohexyl]carbamate

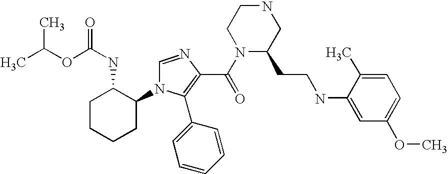

MS (ESI+, m/e) 603 (M+1)

### Example 669

### Isopropyl [(1S,2S)-2-(4-{[(2R)-2-{2-[(3-methoxy-2-methylphenyl)amino]ethyl}piperazin-1-yl]carbonyl}-5-phenyl-1H-imidazol-1-yl)cyclohexyl]carbamate

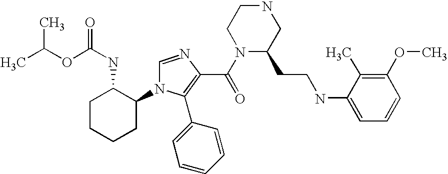

MS (ESI+, m/e) 603 (M+1)

### Example 670

### Ethyl [(1S,2S)-2-(4-{[(2R)-2-{2-[(5-methoxy-2-methylphenyl)amino]ethyl}piperazin-1-yl]carbonyl}-5-phenyl-1H-imidazol-1-yl)cyclohexyl]carbamate

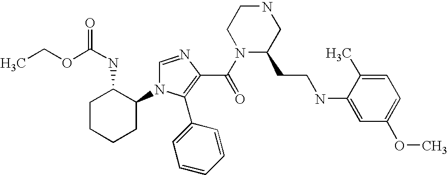

MS (ESI+, m/e) 589 (M+1)

### Example 671

### Ethyl [(1S,2S)-2-(4-{[(2R)-2-{2-[(3-methoxy-2-methylphenyl)amino]ethyl}piperazin-1-yl]carbonyl}-5-phenyl-1H-imidazol-1-yl)cyclohexyl]carbamate

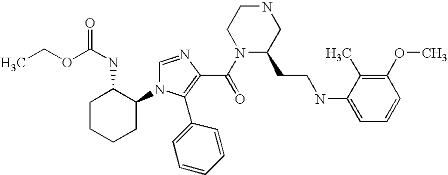

MS (ESI+, m/e) 589 (M+1)

### Example 672

### Methyl [(1S,2S)-2-(4-{[(2R)-2-{2-[(3-methoxy-2-methylphenyl)amino]ethyl}piperazin-1-yl]carbonyl}-5-phenyl-1H-imidazol-1-yl)cyclohexyl]carbamate

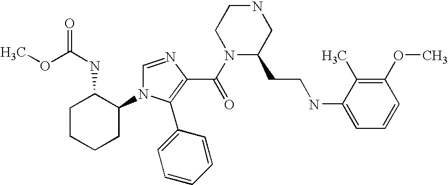

MS (ESI+, m/e) 575 (M+1)

### Example 673

### (1S,2R)-2-(4-{[(2R)-2-{2-[(3-Methoxy-2-methylphenyl)amino]ethyl}piperazin-1-yl]carbonyl}-5-phenyl-1H-imidazol-1-yl)-1-methylcyclohexanol

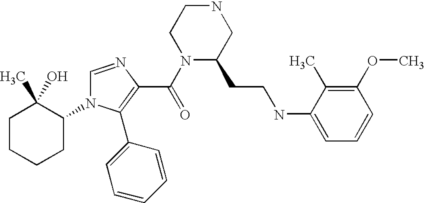

MS (ESI+, m/e) 532 (M+1)

### Example 674

### (1R,2R)-1-(Cyclopropylmethyl)-2-(4-{[(2R)-2-(4-methoxybenzyl)piperazin-1-yl]carbonyl}-5-phenyl-1H-imidazol-1-yl)cyclohexanol

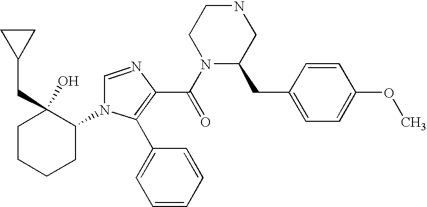

MS (ESI+, m/e) 529 (M+1)

### Example 675

### Isopropyl [(1S,2S)-2-(4-{[(2R)-2-{2-[(2-fluoro-3-methoxyphenyl)amino]ethyl}piperazin-1-yl]carbonyl}-5-phenyl-1H-imidazol-1-yl)cyclohexyl]carbamate

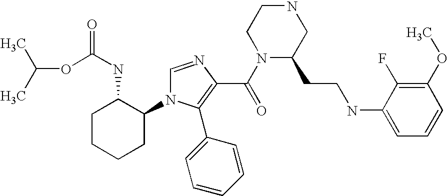

MS (ESI+, m/e) 607 (M+1)

### Example 676

### Cyclobutyl [(1S,2S)-2-(4-{[(2R)-2-{2-[(2-fluoro-3-methoxyphenyl)amino]ethyl}piperazin-1-yl]carbonyl}-5-phenyl-1H-imidazol-1-yl)cyclohexyl]carbamate

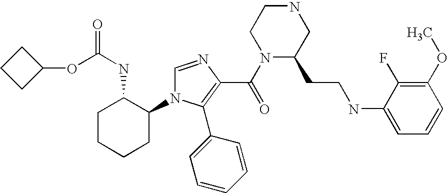

MS (ESI+, m/e) 619 (M+1)

In the same manner as in Example 3 (Method C) except that the treatment of the final compound with 4N hydrogen chloride-ethyl acetate solution was omitted, the following compounds (Examples 677-682) were obtained by isolating as a free amorphous solid.

### Example 677

### 4-{[(2R)-1-({1-[(1R,2R)-2-(Cyclopropylmethyl)-2-hydroxycyclohexyl]-5-phenyl-1H-imidazol-4-yl}carbonyl)piperazin-2-yl]methyl}benzonitrile

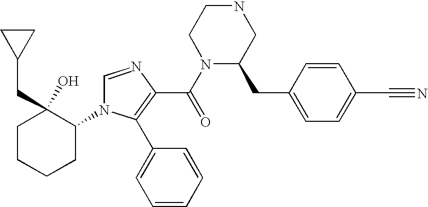

MS (ESI+, m/e) 524 (M+1)

### Example 678

### (1R,2R)-1-(Cyclopropylmethyl)-2-[5-phenyl-4-({(2S)-2-[(5-phenyl-2H-tetrazol-2-yl)methyl]piperazin-1-yl}carbonyl)-1H-imidazol-1-yl]cyclohexanol

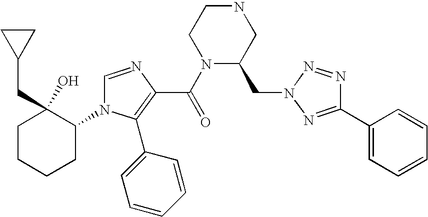

MS (ESI+, m/e) 567 (M+1)

### Example 679

### (1R,2R)-1-(Cyclopropylmethyl)-2-[5-phenyl-4-({(2S)-2-[(2-phenyl-1H-imidazol-1-yl)methyl]piperazin-1-yl}carbonyl)-1H-imidazol-1-yl]cyclohexanol

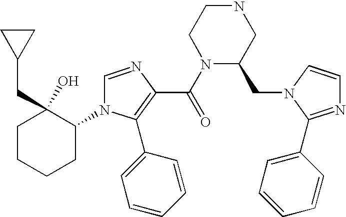

MS (ESI+, m/e) 565 (M+1)

### Example 680

### (1R,2R)-1-(Cyclopropylmethyl)-2-(4-{[(2S)-2-(1H-indazol-1-ylmethyl)piperazin-1-yl]carbonyl}-5-phenyl-1H-imidazol-1-yl)cyclohexanol

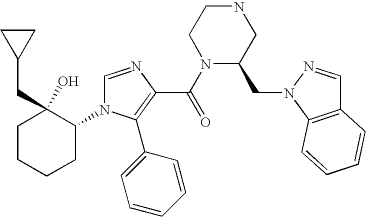

MS (ESI+, m/e) 539 (M+1)

### Example 681

### (1R,2R)-2-(4-{[(2S)-2-(1H-Benzimidazol-1-ylmethyl)piperazin-1-yl]carbonyl}-5-phenyl-1H-imidazol-1-yl)-1-(cyclopropylmethyl)cyclohexanol

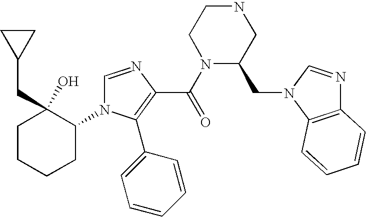

MS (ESI+, m/e) 539 (M+1)

### Example 682

### (1R,2R)-1-(Cyclopropylmethyl)-2-[4-({(2S)-2-[(4-methyl-1H-pyrazol-1-yl)methyl]piperazin-1-yl}carbonyl)-5-phenyl-1H-imidazol-1-yl]cyclohexanol

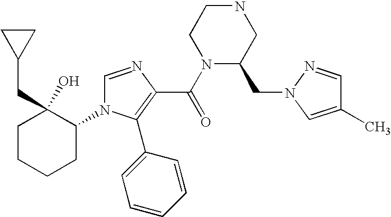

MS (ESI+, m/e) 503 (M+1)

In the same manner as in Example 6 (Method F), the following compound (Example 683) was obtained.

### Example 683

### tert-Butyl [(1S,2S)-2-(4-{[(2R)-2-(3,5-difluorobenzyl)piperazin-1-yl]carbonyl}-5-phenyl-1H-imidazol-1-yl)cyclohexyl]carbamate

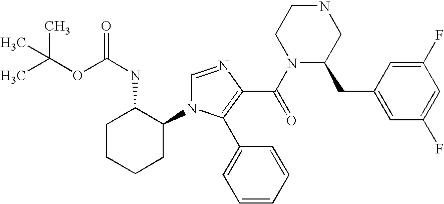

MS (ESI+, m/e) 580 (M+1)

In the same manner as in Example 11 (Method K), the following compounds (Examples 684-692) were obtained.

### Example 684

### Ethyl [(1S,2S)-2-(4-{[(2R)-2-{2-[(5-chloro-2-methylphenyl)amino]ethyl}piperazin-1-yl]carbonyl}-5-phenyl-1H-imidazol-1-yl)cyclohexyl]carbamate

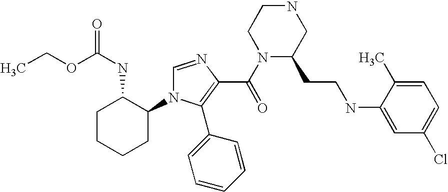

MS (ESI+, m/e) 593 (M+1)

### Example 685

### (1S,2R)-2-(4-{[(2R)-2-{2-[(5-Chloro-2-methylphenyl)amino]ethyl}piperazin-1-yl]carbonyl}-5-phenyl-1H-imidazol-1-yl)-1-(methoxymethyl)cyclohexanol

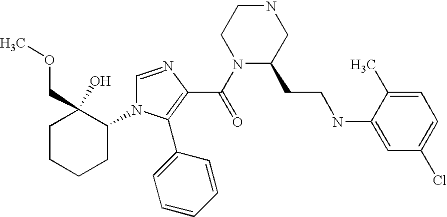

MS (ESI+, m/e) 566 (M+1)

### Example 686

### Ethyl {(1S,2S)-2-[4-({(2R)-2-[2-(2-chloro-4-methylphenoxy)ethyl]piperazin-1-yl}carbonyl)-5-phenyl-1H-imidazol-1-yl]cyclohexyl}carbamate

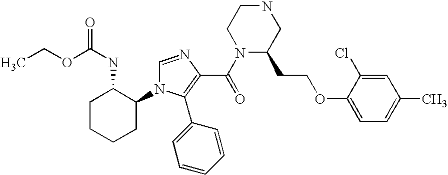

MS (ESI+, m/e) 594 (M+1)

### Example 687

### Ethyl {(1S,2S)-2-[4-({(2R)-2-[2-(4-chloro-2-fluorophenoxy)ethyl]piperazin-1-yl}carbonyl)-5-phenyl-1H-imidazol-1-yl]cyclohexyl}carbamate

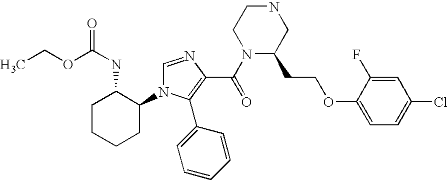

MS (ESI+, m/e) 598 (M+1)

### Example 688

### Ethyl {(1S,2S)-2-[4-({(2R)-2-[2-(2,3-dichlorophenoxy)ethyl]piperazin-1-yl}carbonyl)-5-phenyl-1H-imidazol-1-yl]cyclohexyl}carbamate

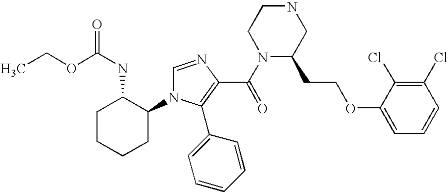

MS (ESI+, m/e) 614 (M+1)

### Example 689

### Ethyl {(1S,2S)-2-[4-({(2R)-2-[2-(4-chlorophenoxy)ethyl]piperazin-1-yl}carbonyl)-5-phenyl-1H-imidazol-1-yl]cyclohexyl}carbamate

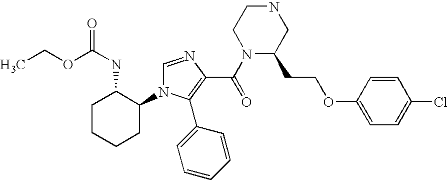

MS (ESI+, m/e) 580 (M+1)

### Example 690

### Methyl {(1S,2S)-2-[4-({(2R)-2-[2-(1-benzothiophen-4-yloxy)ethyl]piperazin-1-yl}carbonyl)-5-phenyl-1H-imidazol-1-yl]cyclohexyl}carbamate

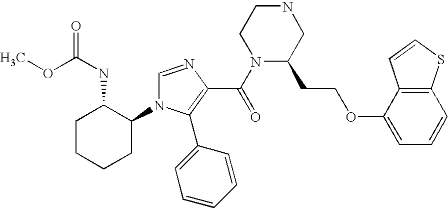

MS (ESI+, m/e) 588 (M+1)

### Example 691

### (1S,2R)-1-(Methoxymethyl)-2-(4-{[(2R)-2-(2-{[2-isopropyl-1,3-benzothiazol-5-yl]oxy}ethyl)piperazin-1-yl]carbonyl}-5-phenyl-1H-imidazol-1-yl)cyclohexanol

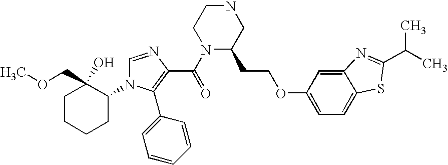

MS (ESI+, m/e) 618 (M+1)

### Example 692

### (1S,2R)-2-(4-{[(2R)-2-{2-[(2-Ethyl-1,3-benzothiazol-5-yl)oxy]ethyl}piperazin-1-yl]carbonyl}-5-phenyl-1H-imidazol-1-yl)-1-(methoxymethyl)cyclohexanol

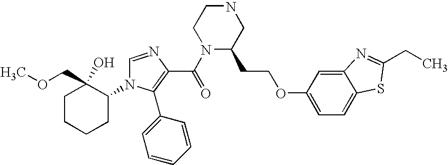

MS (ESI+, m/e) 604 (M+1)

In the same manner as in Example 9 (Method I), the following compounds (Examples 693-762) were obtained.

### Example 693

### (1S,2R)-1-(Methoxymethyl)-2-(4-{[(2R)-2-{2-[3-(3-methoxypropoxy)phenoxy]ethyl}piperazin-1-yl]carbonyl}-5-phenyl-1H-imidazol-1-yl)cyclohexanol

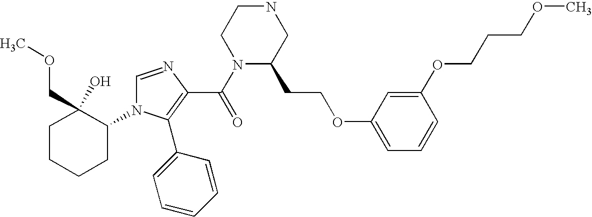

MS (ESI+, m/e) 607 (M+1)

### Example 694

### (1S,2R)-2-(4-{[(2R)-2-{2-[3-(2-Methoxyethoxy)phenoxy]ethyl}piperazin-1-yl]carbonyl}-5-phenyl-1H-imidazol-1-yl)-1-(methoxymethyl)cyclohexanol

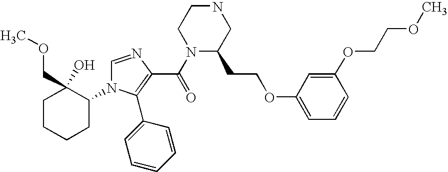

MS (ESI+, m/e) 593 (M+1)

### Example 695

### Ethyl {(1S,2S)-2-[4-({(2R)-2-[2-(2,3-dihydro-1-benzofuran-6-yloxy)ethyl]piperazin-1-yl}carbonyl)-5-phenyl-1H-imidazol-1-yl]cyclohexyl}carbamate

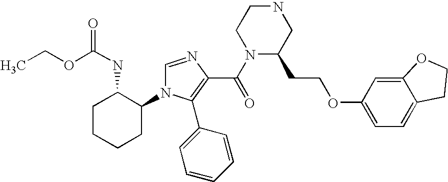

MS (ESI+, m/e) 588 (M+1)

### Example 696

### Ethyl {(1S,2S)-2-[4-({(2R)-2-[2-(2,3-dihydro-1-benzofuran-5-yloxy)ethyl]piperazin-1-yl}carbonyl)-5-phenyl-1H-imidazol-1-yl]cyclohexyl}carbamate

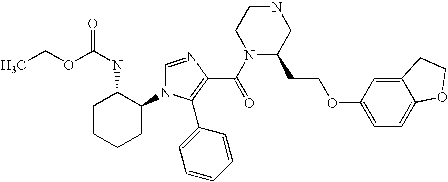

MS (ESI+, m/e) 588 (M+1)

### Example 697

### Ethyl {(1S,2S)-2-[4-({(2R)-2-[2-(5-methoxy-2-methylphenoxy)ethyl]piperazin-1-yl}carbonyl)-5-phenyl-1H-imidazol-1-yl]cyclohexyl}carbamate

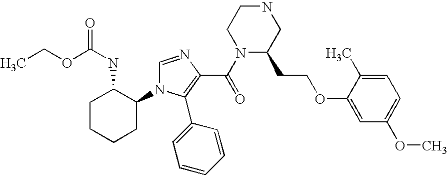

MS (ESI+, m/e) 590 (M+1)

### Example 698

### Methyl {(1S,2S)-2-[4-({(2R)-2-[2-(5-methoxy-2-methylphenoxy)ethyl]piperazin-1-yl}carbonyl)-5-phenyl-1H-imidazol-1-yl]cyclohexyl}carbamate

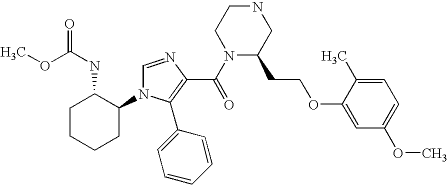

MS (ESI+, m/e) 576 (M+1)

### Example 699

### Ethyl {(1S,2S)-2-[4-({(2R)-2-[2-(5-chloro-2-methylphenoxy)ethyl]piperazin-1-yl}carbonyl)-5-phenyl-1H-imidazol-1-yl]cyclohexyl}carbamate

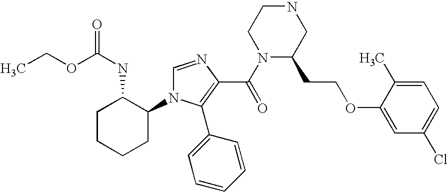

MS (ESI+, m/e) 595 (M+1)

### Example 700

### Ethyl {(1S,2S)-2-[4-({(2R)-2-[2-(2-chloro-5-methoxyphenoxy)ethyl]piperazin-1-yl}carbonyl)-5-phenyl-1H-imidazol-1-yl]cyclohexyl}carbamate

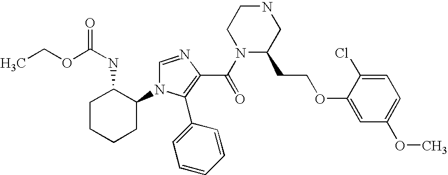

MS (ESI+, m/e) 611 (M+1)

### Example 701

### Ethyl {(1S,2S)-2-[4-({(2R)-2-[2-(2-chloro-4-methoxyphenoxy)ethyl]piperazin-1-yl}carbonyl)-5-phenyl-1H-imidazol-1-yl]cyclohexyl}carbamate

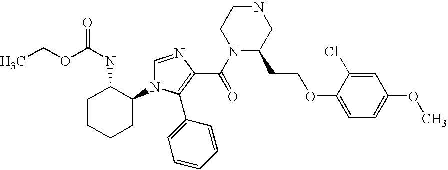

MS (ESI+, m/e) 611 (M+1)

### Example 702

### (1S,2R)-2-(4-{[(2R)-2-{2-[(2-Cyclopropyl-1,3-benzoxazol-5-yl)oxy]ethyl}piperazin-1-yl]carbonyl}-5-phenyl-1H-imidazol-1-yl)-1-(methoxymethyl)cyclohexanol

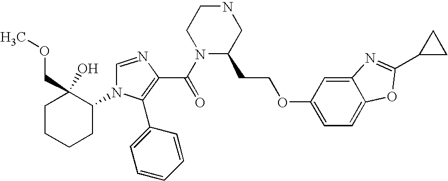

MS (ESI+, m/e) 600 (M+1)

### Example 703

### Ethyl [(1S,2S)-2-(4-{[(2R)-2-{2-[(2-cyclopropyl-1,3-benzoxazol-5-yl)oxy]ethyl}piperazin-1-yl]carbonyl}-5-phenyl-1H-imidazol-1-yl)cyclohexyl]carbamate

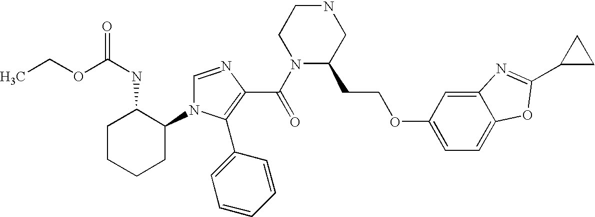

MS (ESI+, m/e) 627 (M+1)

### Example 704

### Ethyl [(1S,2S)-2-(4-{[(2R)-2-{2-[(2,7-dimethyl-1,3-benzoxazol-6-yl)oxy]ethyl}piperazin-1-yl]carbonyl}-5-phenyl-1H-imidazol-1-yl)cyclohexyl]carbamate

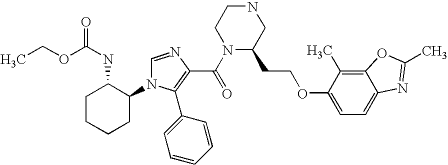

MS (ESI+, m/e) 615 (M+1)

### Example 705

### Ethyl [(1S,2S)-2-(4-{[(2R)-2-{2-[(2-methyl-1,3-benzoxazol-6-yl)oxy]ethyl}piperazin-1-yl]carbonyl}-5-phenyl-1H-imidazol-1-yl)cyclohexyl]carbamate

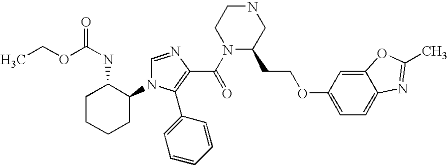

MS (ESI+, m/e) 601 (M+1)

### Example 706

### Ethyl [(1S,2S)-2-(4-{[(2R)-2-{2-[(2-ethyl-1,3-benzoxazol-5-yl)oxy]ethyl}piperazin-1-yl]carbonyl}-5-phenyl-1H-imidazol-1-yl)cyclohexyl]carbamate

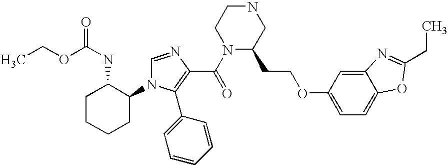

MS (ESI+, m/e) 615 (M+1)

### Example 707

### Methyl [(1S,2S)-2-(4-{[(2R)-2-{2-[(2-ethyl-1,3-benzoxazol-5-yl)oxy]ethyl}piperazin-1-yl]carbonyl}-5-phenyl-1H-imidazol-1-yl)cyclohexyl]carbamate

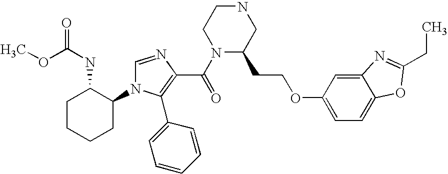

MS (ESI+, m/e) 601 (M+1)

### Example 708

### Methyl [(1S,2S)-2-(4-{[(2R)-2-{2-[(2-methyl-1,3-benzoxazol-6-yl)oxy]ethyl}piperazin-1-yl]carbonyl}-5-phenyl-1H-imidazol-1-yl)cyclohexyl] carbamate

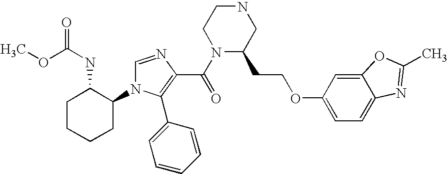

MS (ESI+, m/e) 587 (M+1)

### Example 709

### (1S,2R)-2-(4-{[(2R)-2-{2-[(2-Ethyl-1,3-benzoxazol-5-yl)oxy]ethyl}piperazin-1-yl]carbonyl}-5-phenyl-1H-imidazol-1-yl)-1-(methoxymethyl)cyclohexanol

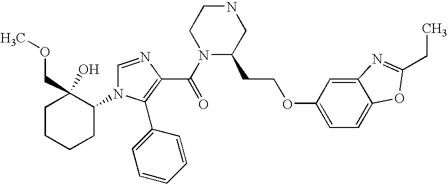

MS (ESI+, m/e) 588 (M+1)

### Example 710

### (1S,2R)-2-(4-{[(2R)-2-{2-[(2,7-Dimethyl-1,3-benzoxazol-6-yl)oxy]ethyl}piperazin-1-yl]carbonyl}-5-phenyl-1H-imidazol-1-yl)-1-(methoxymethyl)cyclohexanol

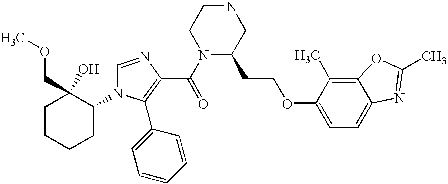

MS (ESI+, m/e) 588 (M+1)

### Example 711

### (1S,2R)-1-(Methoxymethyl)-2-(4-{[(2R)-2-{2-[(2-methyl-1,3-benzoxazol-6-yl)oxy]ethyl}piperazin-1-yl]carbonyl}-5-phenyl-1H-imidazol-1-yl)cyclohexanol

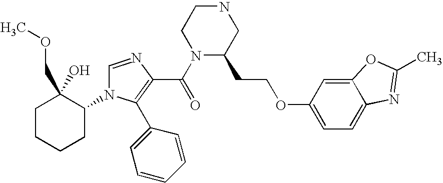

MS (ESI+, m/e) 574 (M+1)

### Example 712

### Methyl [(1S,2S)-2-(4-{[(2R)-2-{2-[3,5-bis(trifluoromethyl)phenoxy]ethyl}piperazin-1-yl]carbonyl}-5-phenyl-1H-imidazol-1-yl)cyclohexyl]carbamate

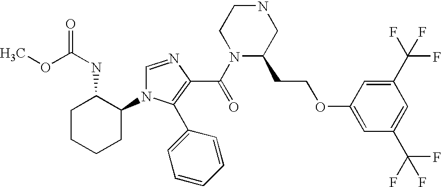

MS (ESI+, m/e) 688 (M+1)

### Example 713

### Methyl [(1S,2S)-2-(4-{[(2R)-2-{2-[3-fluoro-5-(trifluoromethyl)phenoxy]ethyl}piperazin-1-yl]carbonyl}-5-phenyl-1H-imidazol-1-yl)cyclohexyl]carbamate

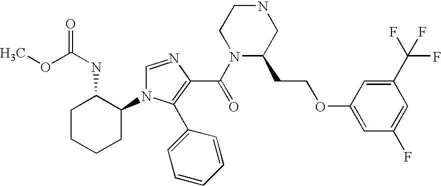

MS (ESI+, m/e) 618 (M+1)

### Example 714

### Methyl {(1S,2S)-2-[4-({(2R)-2-[2-(3,5-difluorophenoxy)ethyl]piperazin-1-yl}carbonyl)-5-phenyl-1H-imidazol-1-yl]cyclohexyl}carbamate

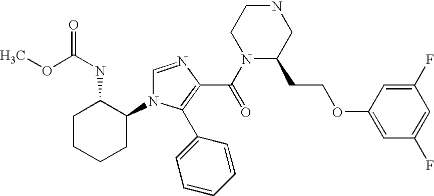

MS (ESI+, m/e) 568 (M+1)

### Example 715

### Methyl [6-(2-{(2R)-1-[(1-{(1S,2S)-2-[(methoxycarbonyl)amino]cyclohexyl}-5-phenyl-1H-imidazol-4-yl)carbonyl]piperazin-2-yl}ethoxy)-2,3-dihydro-1-benzofuran-3-yl]acetate

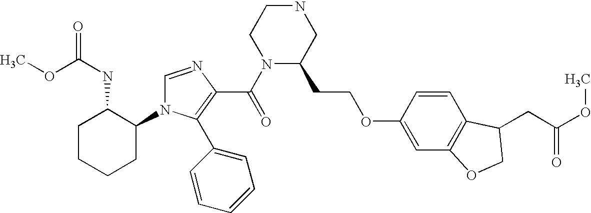

MS (ESI+, m/e) 646 (M+1)

### Example 716

### Methyl {(1S,2S)-2-[4-({(2R)-2-[2-(4-tert-butylphenoxy)ethyl]piperazin-1-yl}carbonyl)-5-phenyl-1H-imidazol-1-yl]cyclohexyl}carbamate

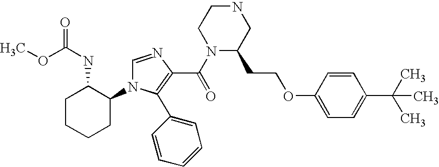

MS (ESI+, m/e) 588 (M+1)

### Example 717

### Methyl {(1S,2S)-2-[4-({(2R)-2-[2-(3,4-dimethylphenoxy)ethyl]piperazin-1-yl}carbonyl)-5-phenyl-1H-imidazol-1-yl]cyclohexyl}carbamate

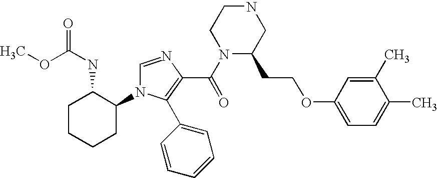

MS (ESI+, m/e) 560 (M+1)

### Example 718

### Methyl {(1S,2S)-2-[4-({(2R)-2-[2-(4-isopropylphenoxy)ethyl]piperazin-1-yl}carbonyl)-5-phenyl-1H-imidazol-1-yl]cyclohexyl}carbamate

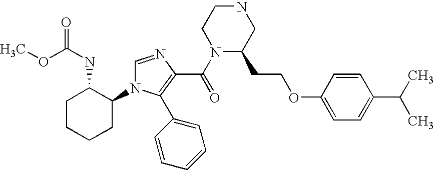

MS (ESI+, m/e) 574 (M+1)

### Example 719

### (1S,2R)-2-{4-[((2R)-2-{2-[(2-Ethyl-7-methyl-1,3-benzoxazol-6-yl)oxy]ethyl}piperazin-1-yl)carbonyl]-5-phenyl-1H-imidazol-1-yl}-1-(methoxymethyl)cyclohexanol

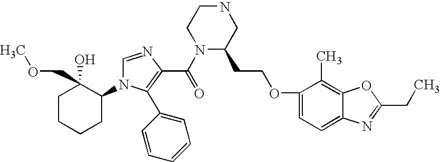

MS (ESI+, m/e) 602 (M+1)

### Example 720

### (1S,2R)-2-{4-[((2R)-2-{2-[(2-Ethyl-1,3-benzoxazol-6-yl)oxy]ethyl}piperazin-1-yl)carbonyl]-5-phenyl-1H-imidazol-1-yl}-1-(methoxymethyl)cyclohexanol

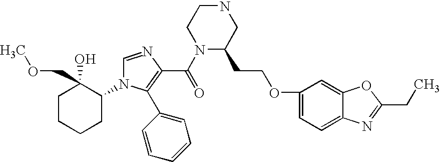

MS (ESI+, m/e) 588 (M+1)

### Example 721

### (1S,2R)-1-Methyl-2-{4-[((2R)-2-{2-[(2-methyl-1,3-benzoxazol-5-yl)oxy]ethyl}piperazin-1-yl)carbonyl]-5-phenyl-1H-imidazol-1-yl}cyclohexanol

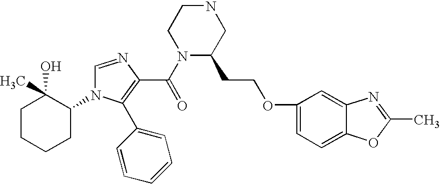

MS (ESI+, m/e) 544 (M+1)

### Example 722

### Ethyl ((1S,2S)-2-{4-[((2R)-2-{2-[(2-methyl-1,3-benzoxazol-5-yl)oxy]ethyl}piperazin-1-yl)carbonyl]-5-phenyl-1H-imidazol-1-yl}cyclohexyl)carbamate

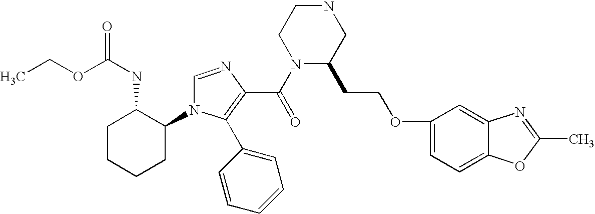

MS (ESI+, m/e) 601 (M+1)

### Example 723

### N-{(1S,2S)-2-[4-({(2R)-2-[2-(3,4-Dimethylphenoxy)ethyl]piperazin-1-yl}carbonyl)-5-phenyl-1H-imidazol-1-yl]cyclohexyl}methanesulfonamide

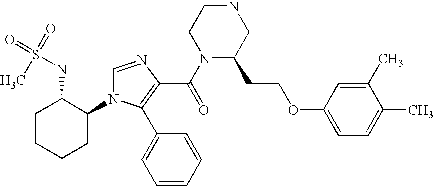

MS (ESI+, m/e) 580 (M+1)

### Example 724

### N-{(1S,2S)-2-[4-({(2R)-2-[2-(Naphthalen-2-yloxy)ethyl]piperazin-1-yl}carbonyl)-5-phenyl-1H-imidazol-1-yl]cyclohexyl}methanesulfonamide

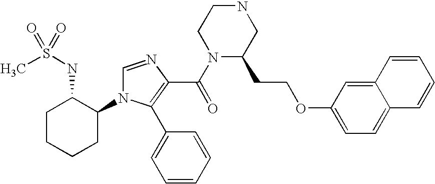

MS (ESI+, m/e) 602 (M+1)

### Example 725

### N-{(1S,2S)-2-[4-({(2R)-2-[2-(4-Methylphenoxy)ethyl]piperazin-1-yl}carbonyl)-5-phenyl-1H-imidazol-1-yl]cyclohexyl}methanesulfonamide

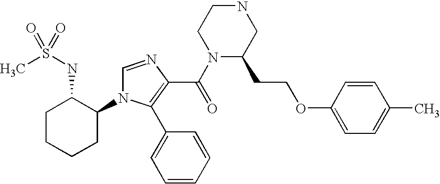

MS (ESI+, m/e) 566 (M+1)

### Example 726

### 2-Methoxyethyl {(1S,2S)-2-[4-({(2R)-2-[2-(naphthalen-2-yloxy)ethyl]piperazin-1-yl}carbonyl)-5-phenyl-1H-imidazol-1-yl]cyclohexyl}carbamate

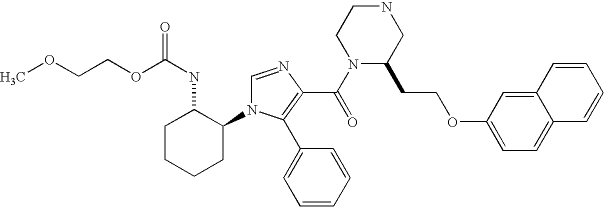

MS (ESI+, m/e) 626 (M+1)

### Example 727

### 2-Methoxyethyl {(1S,2S)-2-[4-({(2R)-2-[2-(4-methylphenoxy)ethyl]piperazin-1-yl}carbonyl)-5-phenyl-1H-imidazol-1-yl]cyclohexyl}carbamate

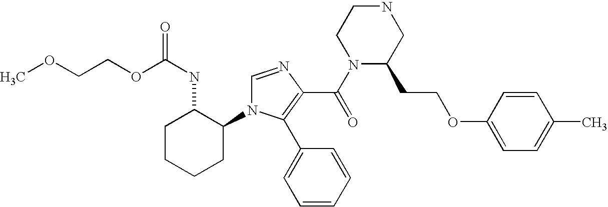

MS (ESI+, m/e) 590 (M+1)

### Example 728

### Cyclobutyl {(1S,2S)-2-[5-phenyl-4-({(2R)-2-[2-(phenylamino)ethyl]piperazin-1-yl}carbonyl)-1H-imidazol-1-yl]cyclohexyl}carbamate

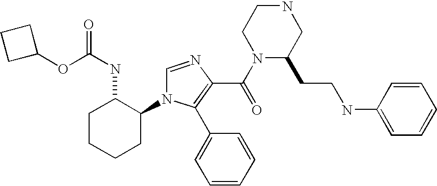

MS (ESI+, m/e) 571 (M+1)

### Example 729

### Cyclobutyl [(1S,2S)-2-(4-{[(2R)-2-benzylpiperazin-1-yl]carbonyl}-5-phenyl-1H-imidazol-1-yl)cyclohexyl]carbamate

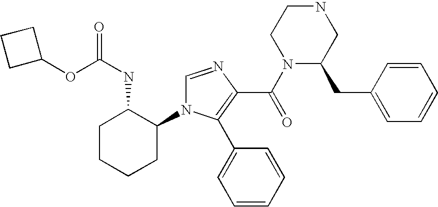

MS (ESI+, m/e) 542 (M+1)

### Example 730

### (1S,2R)-1-(Methoxymethyl)-2-[4-({(2R)-2-[2-(3-methoxy-4-methylphenoxy)ethyl]piperazin-1-yl}carbonyl)-5-phenyl-1H-imidazol-1-yl]cyclohexanol

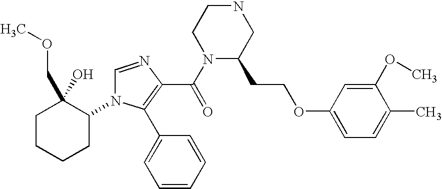

MS (ESI+, m/e) 563 (M+1)

### Example 731

### Methyl {(1S,2S)-2-[4-({(2R)-2-[2-(3-methoxy-4-methylphenoxy)ethyl]piperazin-1-yl}carbonyl)-5-phenyl-1H-imidazol-1-yl]cyclohexyl}carbamate

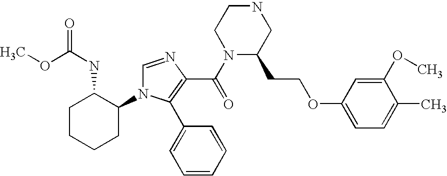

MS (ESI+, m/e) 576 (M+1)

### Example 732

### Ethyl {(1S,2S)-2-[4-({(2R)-2-[2-(3-methoxy-4-methylphenoxy)ethyl]piperazin-1-yl}carbonyl)-5-phenyl-1H-imidazol-1-yl]cyclohexyl}carbamate

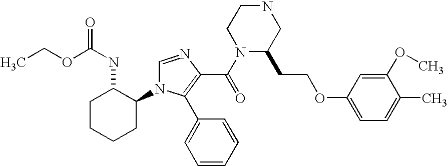

MS (ESI+, m/e) 590 (M+1)

### Example 733

### Ethyl {(1S,2S)-2-[4-({(2R)-2-[2-(4-methylphenoxy)ethyl]piperazin-1-yl}carbonyl)-5-phenyl-1H-imidazol-1-yl]cyclohexyl}carbamate

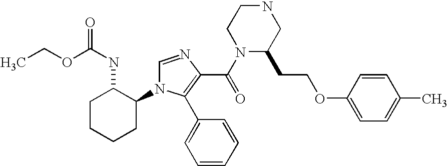

MS (ESI+, m/e) 560 (M+1)

### Example 734

### Ethyl {(1S,2S)-2-[4-({(2R)-2-[2-(naphthalen-2-yloxy)ethyl]piperazin-1-yl}carbonyl)-5-phenyl-1H-imidazol-1-yl]cyclohexyl}carbamate

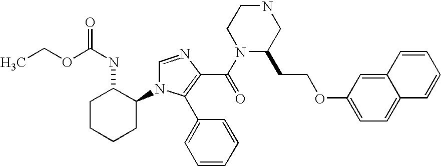

MS (ESI+, m/e) 596 (M+1)

### Example 735

### Methyl {(1S,2S)-2-[4-({(2R)-2-[2-(4-methylphenoxy)ethyl]piperazin-1-yl}carbonyl)-5-phenyl-1H-imidazol-1-yl]cyclohexyl}carbamate

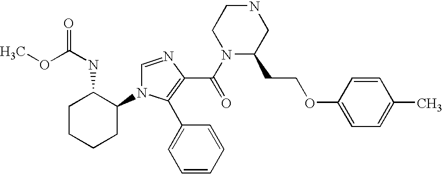

MS (ESI+, m/e) 546 (M+1)

### Example 736

### Methyl {(1S,2S)-2-[4-({(2R)-2-[2-(naphthalen-2-yloxy)ethyl]piperazin-1-yl}carbonyl)-5-phenyl-1H-imidazol-1-yl]cyclohexyl}carbamate

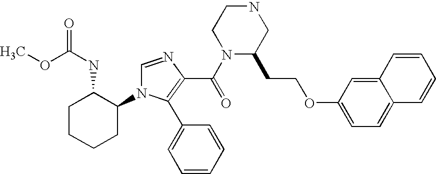

MS (ESI+, m/e) 582 (M+1)

### Example 737

### Methyl {(1S,2S)-2-[4-({(2R)-2-[2-(2,3-dihydro-1H-inden-2-yloxy)ethyl]piperazin-1-yl}carbonyl)-5-phenyl-1H-imidazol-1-yl]cyclohexyl}carbamate

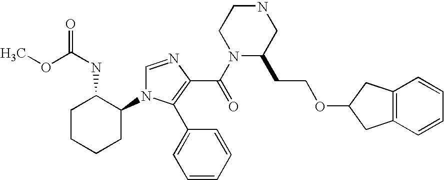

MS (ESI+, m/e) 572 (M+1)

### Example 738

### (1S,2R)-2-[4-({(2R)-2-[2-(2,3-Dihydro-1H-inden-2-yloxy)ethyl]piperazin-1-yl}carbonyl)-5-phenyl-1H-imidazol-1-yl]-1-(methoxymethyl)cyclohexanol

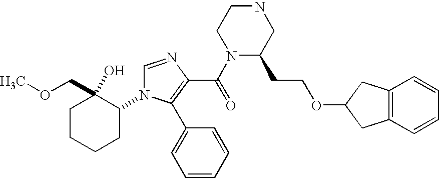

MS (ESI+, m/e) 559 (M+1)

### Example 739

### Methyl {(1S,2S)-2-[4-({(2R)-2-[2-(2,6-difluorophenoxy)ethyl]piperazin-1-yl}carbonyl)-5-phenyl-1H-imidazol-1-yl]cyclohexyl}carbamate

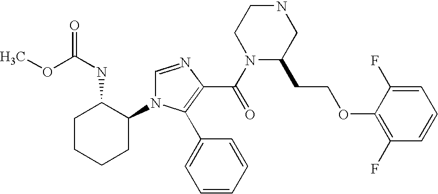

MS (ESI+, m/e) 568 (M+1)

### Example 740

### (1S,2R)-2-[4-({(2R)-2-[2-(2,6-Difluorophenoxy)ethyl]piperazin-1-yl}carbonyl)-5-phenyl-1H-imidazol-1-yl]-1-(methoxymethyl)cyclohexanol

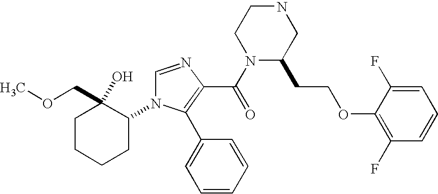

MS (ESI+, m/e) 555 (M+1)

### Example 741

### 6-{2-[(2R)-1-({1-[(1R,2S)-2-Hydroxy-2-(methoxymethyl)cyclohexyl]-5-phenyl-1H-imidazol-4-yl}carbonyl)piperazin-2-yl]ethoxy}-1-(3-methoxypropyl)-3,4-dihydroquinolin-2(1H)-one

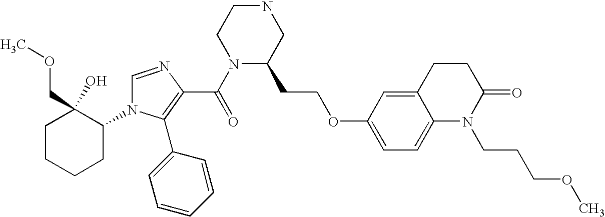

MS (ESI+, m/e) 660 (M+1)

### Example 742

### 6-{2-[(2R)-1-({1-[(1R,2R)-2-(Cyclopropylmethyl)-2-hydroxycyclohexyl]-5-phenyl-1H-imidazol-4-yl}carbonyl)piperazin-2-yl]ethoxy}-1-(2-methoxyethyl)-3,4-dihydroquinolin-2(1H)-one

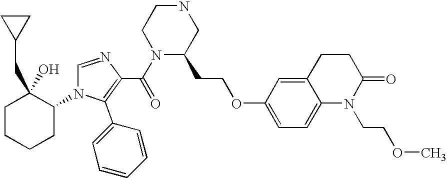

MS (ESI+, m/e) 655 (M+1)

### Example 743

### 6-{2-[(2R)-1-({1-[(1R,2R)-2-(Cyclopropylmethyl)-2-hydroxycyclohexyl]-5-phenyl-1H-imidazol-4-yl}carbonyl)piperazin-2-yl]ethoxy}-1-(3-methoxypropyl)-3,4-dihydroquinolin-2(1H)-one

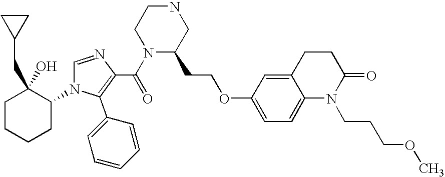

MS (ESI+, m/e) 670 (M+1)

### Example 744

### 6-{2-[(2R)-1-({1-[(1R,2R)-2-(Cyclopropylmethyl)-2-hydroxycyclohexyl]-5-phenyl-1H-imidazol-4-yl}carbonyl)piperazin-2-yl]ethoxy}-2H-1,4-benzoxazin-3(4H)-one

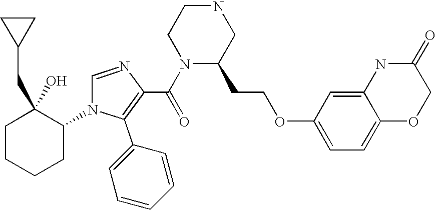

MS (ESI+, m/e) 600 (M+1)

### Example 745

### (1R,2R)-2-(4-{[(2S)-2-(1H-Benzotriazol-1-ylmethyl)piperazin-1-yl]carbonyl}-5-phenyl-1H-imidazol-1-yl)-1-(cyclopropylmethyl)cyclohexanol

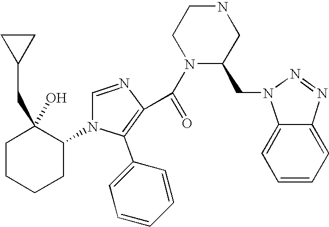

MS (ESI+, m/e) 540 (M+1)

### Example 746

### (1R,2R)-2-[4-({(2R)-2-[2-(1H-Benzotriazol-1-yl)ethyl]piperazin-1-yl}carbonyl)-5-phenyl-1H-imidazol-1-yl]-1-(cyclopropylmethyl)cyclohexanol

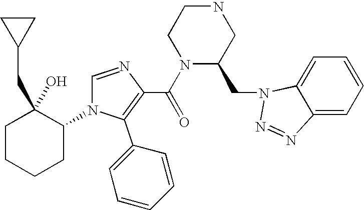

MS (ESI+, m/e) 554 (M+1)

### Example 747

### (1R,2R)-1-(cyclopropylmethyl)-2-(4-{[(2R)-2-{2-[(1,2-dimethyl-1H-benzimidazol-5-yl)oxy]ethyl}piperazin-1-yl]carbonyl}-5-phenyl-1H-imidazol-1-yl)cyclohexanol

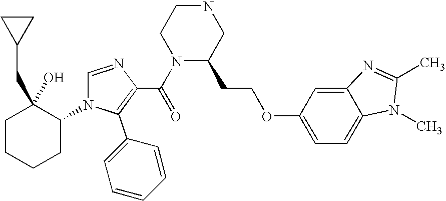

MS (ESI+, m/e) 597 (M+1)

### Example 748

### (1R,2R)-2-[4-({(2R)-2-[2-(1H-Benzimidazol-1-yl)ethyl]piperazin-1-yl}carbonyl)-5-phenyl-1H-imidazol-1-yl]-1-(cyclopropylmethyl)cyclohexanol

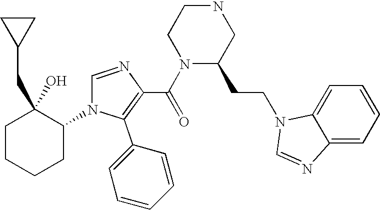

MS (ESI+, m/e) 553 (M+1)

### Example 749

### (1S,2R)-2-[4-({(2R)-2-[2-(3,4-Dimethoxyphenoxy)ethyl]piperazin-1-yl}carbonyl)-5-phenyl-1H-imidazol-1-yl]-1-(methoxymethyl)cyclohexanol

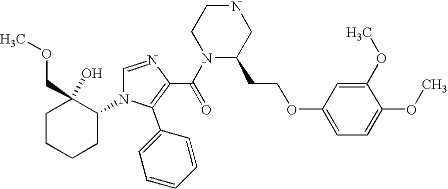

MS (ESI+, m/e) 579 (M+1)

### Example 750

### 6-{2-[(2R)-1-({1-[(1R,2R)-2-(Cyclopropylmethyl)-2-hydroxycyclohexyl]-5-phenyl-1H-imidazol-4-yl}carbonyl)piperazin-2-yl]ethoxy}-1,3-benzoxazol-2(3H)-one

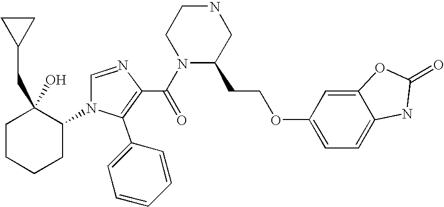

MS (ESI+, m/e) 586 (M+1)

### Example 751

### 7-{2-[(2R)-1-({1-[(1R,2R)-2-(Cyclopropylmethyl)-2-hydroxycyclohexyl]-5-phenyl-1H-imidazol-4-yl}carbonyl)piperazin-2-yl]ethoxy}-3,4-dihydroquinolin-2(1H)-one

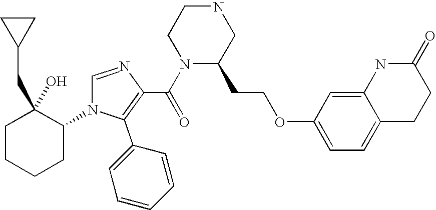

MS (ESI+, m/e) 598 (M+1)

### Example 752

### 5-{2-[(2R)-1-({1-[(1R,2R)-2-(Cyclopropylmethyl)-2-hydroxycyclohexyl]-5-phenyl-1H-imidazol-4-yl}carbonyl)piperazin-2-yl]ethoxy}-3,4-dihydroisoquinolin-1(2H)-one

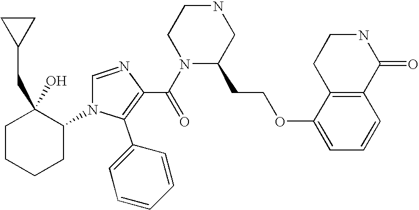

MS (ESI+, m/e) 598 (M+1)

### Example 753

### (1S,2R)-2-(4-{[(2R)-2-{2-[(2,5-Dimethylphenyl)amino]ethyl}piperazin-1-yl]carbonyl}-5-phenyl-1H-imidazol-1-yl)-1-(methoxymethyl)cyclohexanol

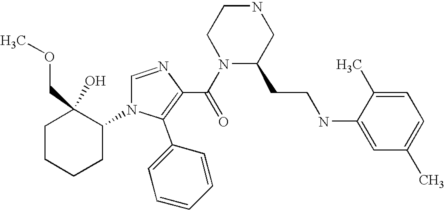

MS (ESI+, m/e) 546 (M+1)

### Example 754

### (1S,2R)-2-(4-{[(2R)-2-{2-[(5-Fluoro-2-methylphenyl)amino]ethyl}piperazin-1-yl]carbonyl}-5-phenyl-1H-imidazol-1-yl)-1-(methoxymethyl)cyclohexanol

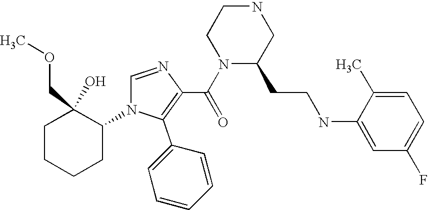

MS (ESI+, m/e) 550 (M+1)

### Example 755

### 6-{2-[(2R)-1-({1-[(1R,2R)-2-(Cyclopropylmethyl)-2-hydroxycyclohexyl]-5-phenyl-1H-imidazol-4-yl}carbonyl)piperazin-2-yl]ethoxy}-3,4-dihydroquinolin-2(1H)-one

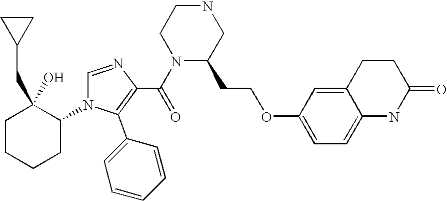

MS (ESI+, m/e) 598 (M+1)

### Example 756

### Ethyl {(1S,2S)-2-[4-({(2R)-2-[2-(3-methoxy-2-methylphenoxy)ethyl]piperazin-1-yl}carbonyl)-5-phenyl-1H-imidazol-1-yl]cyclohexyl}carbamate

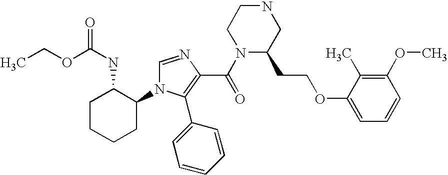

MS (ESI+, m/e) 590 (M+1)

### Example 757

### Ethyl {(1S,2S)-2-[4-({(2R)-2-[2-(2-fluoro-4-methylphenoxy)ethyl]piperazin-1-yl}carbonyl)-5-phenyl-1H-imidazol-1-yl]cyclohexyl}carbamate

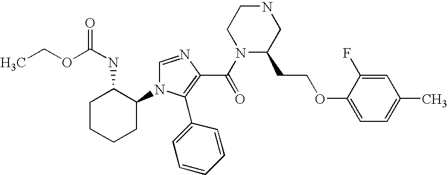

MS (ESI+, m/e) 578 (M+1)

### Example 758

### (1S,2R)-2-(4-{[(2R)-2-{2-[(1,2-Dimethyl-1H-indol-5-yl)oxy]ethyl}piperazin-1-yl]carbonyl}-5-phenyl-1H-imidazol-1-yl)-1-(methoxymethyl)cyclohexanol

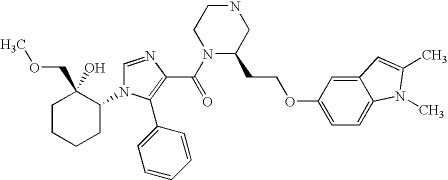

MS (ESI+, m/e) 586 (M+1)

### Example 759

### (1R,2R)-1-(Cyclopropylmethyl)-2-[4-({(2R)-2-[2-(2,3-dihydrofuro[3,2-b]pyridin-5-ylamino)ethyl]piperazin-1-yl}carbonyl)-5-phenyl-1H-imidazol-1-yl]cyclohexanol

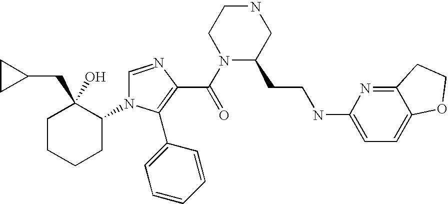

MS (ESI+, m/e) 571 (M+1)

### Example 760

### Methyl {(1S,2S)-2-[4-({(2R)-2-[2-(2-fluoro-3-methoxyphenoxy)ethyl]piperazin-1-yl}carbonyl)-5-phenyl-1H-imidazol-1-yl]cyclohexyl}carbamate

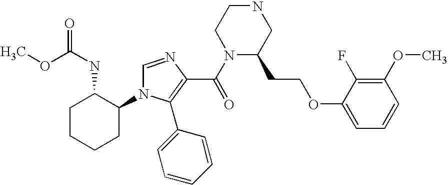

MS (ESI+, m/e) 580 (M+1)

### Example 761

### Methyl [(1S,2S)-2-(4-{[(2R)-2-{2-[(2-fluoro-3-methoxyphenyl)amino]ethyl}piperazin-1-yl]carbonyl}-5-phenyl-1H-imidazol-1-yl)cyclohexyl]carbamate

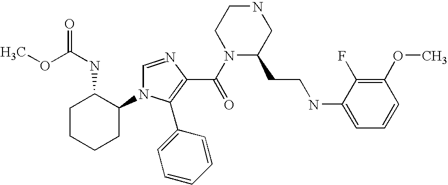

MS (ESI+, m/e) 579 (M+1)

### Example 762

### (1R,2R)-1-(Cyclopropylmethyl)-2-(4-{[(2R)-2-{2-[(2-ethyl-1,3-benzoxazol-5-yl)amino]ethyl}piperazin-1-yl]carbonyl}-5-phenyl-1H-imidazol-1-yl)cyclohexanol

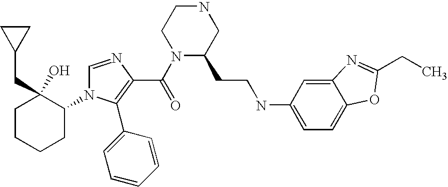

MS (ESI+, m/e) 597 (M+1)

In the same manner as in Example 9 (Method I) except that the treatment of the final compound with 4N hydrogen chloride-ethyl acetate solution was omitted, the following compounds (Examples 763-766) were obtained by isolating as a free amorphous solid.

### Example 763

### (1S,2R)-2-{4-[((2R)-2-{2-[(2,7-Dimethyl-1,3-benzoxazol-6-yl)oxy]ethyl}piperazin-1-yl)carbonyl]-5-phenyl-1H-imidazol-1-yl}-1-(methoxymethyl)cyclohexanol

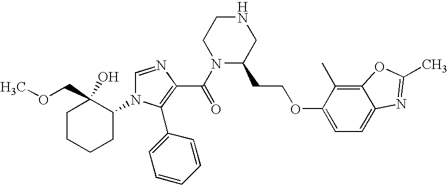

MS (ESI+, m/e) 588 (M+1)

### Example 764

### (1S,2R)-2-{4-[((2R)-2-{2-[(2-Cyclopropyl-1,3-benzoxazol-5-yl)oxy]ethyl}piperazin-1-yl)carbonyl]-5-phenyl-1H-imidazol-1-yl}-1-(methoxymethyl)cyclohexanol

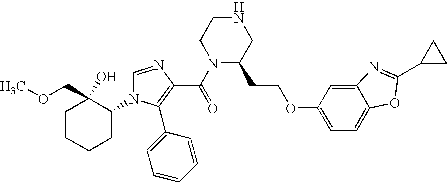

MS (ESI+, m/e) 600 (M+1)

### Example 765

### (1S,2R)-2-{4-[((2R)-2-{2-[(2-Ethyl-1,3-benzoxazol-5-yl)oxy]ethyl}piperazin-1-yl)carbonyl]-5-phenyl-1H-imidazol-1-yl}-1-(methoxymethyl)cyclohexanol

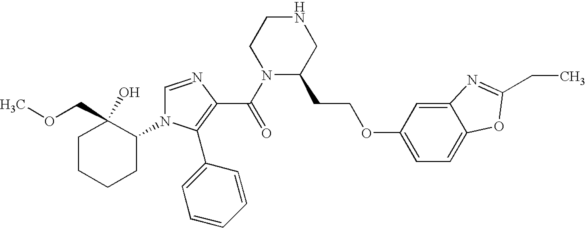

MS (ESI+, m/e) 588 (M+1)

### Example 766

### Methyl {(1S,2S)-2-[4-({(2R)-2-[2-(2-naphthyloxy)ethyl]piperazin-1-yl}carbonyl)-5-phenyl-1H-imidazol-1-yl]cyclohexyl}carbamate

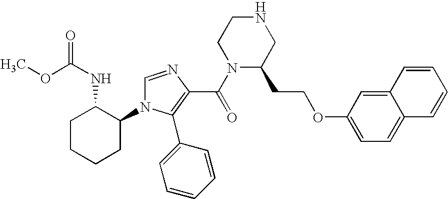

MS (ESI+, m/e) 588 (M+1)

### Example 767

### Ethyl [(1S,2S)-2-(4-{[(2R)-2-(3,5-difluorobenzyl)piperazin-1-yl]carbonyl}-5-phenyl-1H-imidazol-1-yl)cyclohexyl]carbamate succinate

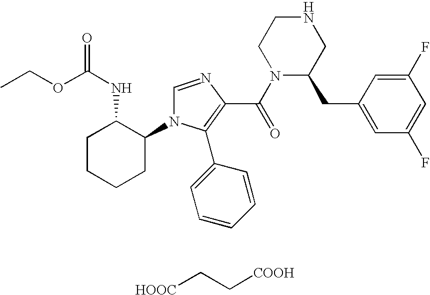

Ethyl [(1S,2S)-2-(4-{[(2R)-2-(3,5-difluorobenzyl)piperazin-1-yl]carbonyl}-5-phenyl-1H-imidazol-1-yl)cyclohexyl]carbamate (1.00 g) and succinic acid (0.21 g) were dissolved in ethanol (10 ml) while heating (80° C.), and the mixture was cooled to room temperature without stirring and stood still at room temperature for 2 days. The crystals were collected by filtration, washed with a small amount of ethanol, and dried under reduced pressure to give the object compound as crystals (1.01 g).

melting point: 204-205° C.

### Example 768

### (1S,2R)-2-(4-{[(2R)-2-Benzylpiperazin-1-yl]carbonyl}-5-phenyl-1H-imidazol-1-yl)-1-methylcyclohexanol 0.5 fumarate

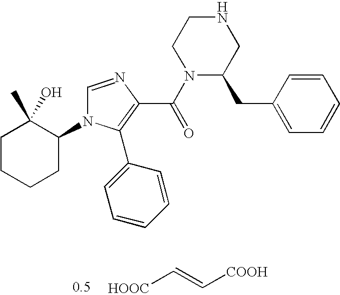

(1S,2R)-2-(4-{[(2R)-2-Benzylpiperazin-1-yl]carbonyl}-5-phenyl-1H-imidazol-1-yl)-1-methylcyclohexanol (0.11 g) and fumaric acid (0.027 g) were dissolved in ethanol (5 ml) while heating (80° C.), and the mixture was cooled to room temperature without stirring and stood still at room temperature for 15 hours. The crystals were collected by filtration, washed with a small amount of ethyl acetate, and dried under reduced pressure to give the object compound as crystals (0.091 g).

melting point: 191° C.

### Example 769

### (1S,2R)-2-(4-{[(2R)-2-Benzylpiperazin-1-yl]carbonyl}-5-phenyl-1H-imidazol-1-yl)-1-methylcyclohexanol 0.5 succinate

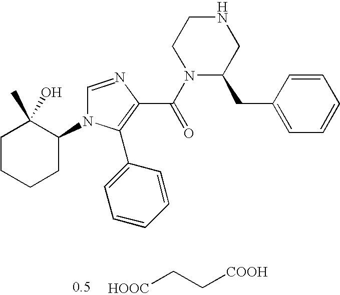

(1S,2R)-2-(4-{[(2R)-2-Benzylpiperazin-1-yl]carbonyl}-5-phenyl-1H-imidazol-1-yl)-1-methylcyclohexanol (0.11 g) and succinic acid (0.028 g) were dissolved in ethanol (5 ml) while heating (80° C.), and the mixture was cooled to room temperature without stirring and stood still at room temperature for 15 hours. The crystals were collected by filtration, washed with a small amount of ethyl acetate, and dried under reduced pressure to give the object compound as crystals (0.062 g).

melting point: 192° C.

### Preparation Example 1

| | | |
|---|---|---|
| (1) Compound of Example 1 | 10.0 | g |
| (2) Lactose | 70.0 | g |
| (3) Cornstarch | 50.0 | g |
| (4) Soluble starch | 7.0 | g |
| (5) Magnesium stearate | 3.0 | g |

10.0 g of the compound of Example 1 and 3.0 g of magnesium stearate are granulated with 70 ml of an aqueous solution of soluble starch (7.0 g as soluble starch), then and the mixture is dried and mixed with 70.0 g of lactose and 50.0 g of corn starch (any of lactose, corn starch, soluble starch and magnesium stearate is products in conformity to the 14^{th} revision of the Japanese Pharmacopoeia). The mixture is compressed to give tablets.

### Experimental Example 1

Human renin was obtained by expressing preprorenin (1-406) in an animal cell, treating the prorenin (24-406) contained in the culture supernatant with trypsin, and taking the active type (67-406).

### (1) Construction of Renin-Expressing Vector

A plasmid DNA to express human renin in HEK293 cells was prepared as follows. PCR was carried out using human renal cDNA (Clontech Laboratories, Inc., Marathon Ready cDNA) as the template and using two synthetic DNAs (5′-AAGCTTATGGATGGATGGAGA-3′; SEQ ID No.1, and 5′-GGATCCTCAGCGGGCCAAGGC-3′; SEQ ID No.2), and the obtained fragments were cloned using a TOPO TA Cloning Kit (Invitrogen Corp.). The obtained fragments were subcloned into pcDNA3.1(+) that had been cleaved by HindIII and BamHI, thus to obtain a plasmid DNA for human preprorenin expression (pcDNA3.1(+)/hREN).

### (2) Construction of Angiotensinogen-Expressing Vector

A plasmid DNA to express human angiotensinogen in HEK293 cells was prepared as follows. PCR was carried out using human liver cDNA (Clontech Laboratories, Inc., Marathon Ready cDNA) as the template and using two synthetic DNAs (5′-AAGCTTATGCGGAAGCGAGCACCCCAGTCT-3′; SEQ ID No.3, and 5′-GGATCCTCACTTGTCATCGTCGTCCTTGTAGTCTGCTGTGCTCAGCGGGTTGGCCACGC-3′; SEQ ID No.4), and the obtained fragments were cloned using a TOPO TA Cloning Kit (Invitrogen Corp.). The obtained fragments were subcloned into pcDNA3.1(+) that had been cleaved by HindIII and BamHI, thereby to give a plasmid DNA for expression of human angiotensinogen having a FLAGtag on the C-terminal (pcDNA3.1(+)/hAngiotensinogen-FLAG). Then, PCR was carried out using the pcDNA3.1(+)/hAngiotensinogen-FLAG as the template and using two synthetic DNAs (5′-CCTTAAGCTTCCACCATGCGGAAGCGAGCACCCCAGTCT-3′; SEQ ID No.5, and 5′-TTGGATCCTCATGCTGTGCTCAGCGGGTTGGCCACGCGG-3′; SEQ ID No.6), and the obtained fragments were cloned using a TOPO TA Cloning Kit (Invitrogen Corp.). The obtained fragments were subcloned into pcDNA3.1(+) that had been cleaved by HindIII and BamHI, thus to obtain a plasmid DNA for human angiotensinogen expression (pcDNA3.1(+)/hAngiotensinogen).

### (3) Expression of Preprorenin and Purification of Prorenin (24-406)

Expression of human preprorenin was conducted using FreeStyle 293 Expression System (Invitrogen Corp.). According to the manual accompanying the FreeStyle 293 Expression System, the plasmid DNA for human preprorenin expression (pcDNA3.1(+)/hREN) constructed in the above-mentioned (1) was used to conduct transient expression by FreeStyle 293-F cells. After transfection of the plasmid DNA, the cells were subjected to shaking culture under the conditions of 37° C., 8% CO₂ and 125 rpm for 3 days. A 600-ml aliquot of the culture solution was centrifuged at 2,000 rpm for 10 min to recover the culture supernatant containing prorenin (24-406). The culture supernatant was concentrated by ultrafiltration using a PM10 membrane (Millipore, Inc.) to a volume of about 50 ml, and then was dialyzed against 20 mM Tris-hydrochloric acid (pH 8.0). The dialyzate was fed to a 6-ml RESOURCE Q column (GE Healthcare) equilibrated with 20 mM Tris-hydrochloric acid (pH 8.0) at a flow rate of 3 ml/min to adsorb the prorenin (24-406). After washing the column with the buffer solution used in the equilibration, elution was carried out by means of a linear concentration gradient of sodium chloride from 0 M to 0.4 M. The fraction containing prorenin (24-406) was collected and concentrated using Vivaspin 20 (molecular weight cut off 10,000; Vivascience, Inc.) to a volume of about 2 ml.

The concentrated liquid was subjected to gel filtration chromatography using HiLoad 16/60 Superdex 200 pg (GE Healthcare) equilibrated with 20 mM Tris-hydrochloric acid (pH 8.0) containing 0.15 M sodium chloride, at a flow rate of 1.4 ml/min, thus to obtain 3.6 mg of purified prorenin (24-406).

### (4) Purification of Active Type Renin (67-406)

To 3.6 mg of prorenin (24-406) dissolved in 5.2 ml of 0.1 M Tris-hydrochloric acid (pH 8.0), 12 μg of trypsin (Roche Diagnostics Corp.) was added, and the mixture was allowed to react at 28° C. for 55 min to carry out activation of renin. After the reaction, 0.4 ml of immobilized trypsin inhibitor (Pierce Biotechnology, Inc.) was added to remove the trypsin used in the activation by adsorption. The reaction liquid containing the active type renin was concentrated using Vivaspin 20 (molecular weight cut off 10,000, Vivascience, Inc.), and was diluted with 20 mM Tris-hydrochloric acid (pH 8.0). The diluted liquid was fed to a TSKgel DEAE-5PW column (7.5 mm I.D.×75 mm, Tosoh Corp.) equilibrated with 20 mM Tris-hydrochloric acid (pH 8.0) at a flow rate of 1 ml/min to adsorb the active type renin (67-406). The column was washed with the buffer solution used for the equilibration, and then elution was carried out by means of a sodium chloride linear concentration gradient from 0 M to 0.3 M, thus to obtain 1.5 mg of a purified product of active type renin (67-406).

### (5) Purification of Angiotensinogen

Expression of human angiotensinogen was conducted using FreeStyle 293 Expression System (Invitrogen Corp.). According to the manual accompanying the FreeStyle 293 Expression System, the plasmid DNA for human angiotensinogen expression (pcDNA3.1(+)/hAngiotensinogen) constructed in the above-mentioned (2) was used to conduct transient expression by FreeStyle 293-F cells. After transfection of the plasmid DNA, the cells were subjected to shaking culture under the conditions of 37° C., 8% CO₂ and 125 rpm for 3 days. A 600-ml aliquot of the culture solution was centrifuged at 2,000 rpm for 10 min to recover the culture supernatant containing angiotensinogen. To the culture supernatant was added ammonium sulfate (30% saturated concentration), and the mixture was thoroughly stirred and centrifuged at 8,000 rpm for 20 min. The obtained supernatant was added to TOYO Pearl butyl 650M (2×5 cm, Tosoh Corporation) equilibrated with 50 mM tris-hydrochloric acid (pH 8.0) containing 30% saturated ammonium sulfate, at a flow rate of 25 ml/min to allow adsorption. After washing with equilibration buffer, angiotensinogen was eluted by linear concentration gradient from the buffer used for equilibration to 20 mM tris-hydrochloric acid (pH 8.0). The eluate containing angiotensinogen was applied to repeated concentration and dilution using Vivaspin 20 (molecular weight cut off 10,000, Vivascience, Inc.), and the buffer was changed to 20 mM tris-hydrochloric acid (pH 8.0). The eluate was fed to a 6-ml RESOURCE Q column (Amersham Biosciences, Inc.) equilibrated with 20 mM Tris-hydrochloric acid (pH 8.0) containing 50 mM sodium chloride at a flow rate of 6 ml/min to adsorb the angiotensinogen. After washing the column with the buffer solution used in the equilibration, elution was carried out by means of a linear concentration gradient of sodium chloride from 50 mM to 400 mM. The fractions containing angiotensinogen were collected and concentrated using Vivaspin 20 (molecular weight cut off 10,000, Vivascience, Inc.) to a volume of about 2 ml. The concentrated liquid was subjected to gel filtration chromatography using HiLoad 26/60 Superdex 200 pg (GE Healthcare) equilibrated with 20 mM Tris-hydrochloric acid (pH 8.0) containing 0.15 M sodium chloride, at a flow rate of 2.0 ml/min, thus to obtain 7.0 mg of purified angiotensinogen.

### (6) Measurement of Renin Inhibition Value—A

As a substrate for renin activity measurement, a substrate peptide (FITC-Acp-Asp-Arg-Val-Tyr-Ile-His-Pro-Phe-His-Leu-Val-Ile-His-Gln-Arg-NH₂; SEQ ID No.8) wherein the N-terminal of a peptide prepared in reference to a partial sequence (Asp-Arg-Val-Tyr-Ile-His-Pro-Phe-His-Leu-Val-Ile-His-Asn-Glu-NH₂; SEQ ID No.7) of human angiotensinogen was bound with epsilon aminocaproic acid (Acp) as a linker and labeled with a fluorescence reagent Fluorescein isothiocyanate (FITC). 2 μl each of the test compound (containing 100% DMSO) was added to each well of a 384-well black plate (Nalge Nunc International Co., Ltd.). Renin was diluted with a buffer solution for reaction (20 mM citric acid-sodium citrate (pH 6.0)) to a concentration of 4.7 nM, and 30 μl each of the dilution was added to each well. The dilution was left to stand at 37° C. for 10 min, and then 8 μl of each of a 25 μM solution of substrate peptide was added to each well to initiate the reaction. The reaction mixture was left to stand at 37° C. for 30 min, and then 40 μl each of a reaction terminating solution [200 mM Tris-hydrochloric acid (pH 8.0), 0.04% Triton-X 100, 0.4% Coating 3 reagent (Caliper Life Sciences Corp.) and 1 μM CGP-29287 (Bachem Holding AG)] was added to each well to terminate the reaction.

The substrate peptide and the product peptide were separated by a microchip type capillary electrophoresis system 250HTS (Caliper Life Sciences Co., Ltd.), and the rate of reaction [(peak height of product)/(peak height of product+peak height of substrate)×100(%)] was calculated from the ratio of the respective peak height of the peptides obtained by fluorimetric detection (excitation wavelength 457 nm, measurement wavelength 530 nm), and was used as an index of the renin activity.

While the reaction rate of the well where 100% DMSO only was added was taken as 0% inhibition rate, and the reaction rate of the well where 10 μM of CGP-29287 was added was taken as 100% inhibition rate, the renin inhibitory activity of the wells where the test compound (containing 100% DMSO) was added was calculated.

The results are presented in Table 29.

TABLE 29

| | inhibitory activity |
|---|---|
| Ex. No. | (%) at 1 μM |
| 1 | 96 |
| 4 | 99 |
| 6 | 98 |
| 7 | 100 |
| 15 | 96 |
| 349 | 101 |
| 352 | 101 |
| 357 | 100 |
| 358 | 103 |
| 360 | 99 |
| 361 | 100 |
| 363 | 98 |
| 367 | 99 |
| 378 | 99 |
| 379 | 99 |
| 380 | 100 |
| 383 | 100 |
| 387 | 109 |
| 389 | 106 |
| 390 | 105 |
| 391 | 109 |

It can be seen from the results of Table 29 that compound (I) of the present invention has a superior renin inhibitory activity as evidenced by an IC₅₀ value of 1 μM or less.

### (7) Measurement of Renin Inhibition Value—B

As a substrate for renin activity measurement, the angiotensinogen mentioned in (5) above was used. 1 μl each of the test compound (containing 100% DMSO) was added to each well of a 384-well plate (ABgene). Renin was diluted with a buffer solution for reaction (20 mM sodium phosphate (pH 7.4)) to a concentration of 57 pM, and 14 μl each of the dilution was added to each well. The dilution was left to stand at 37° C. for 10 min, and then 5 μl of each of a 6 μM solution of substrate angiotensinogen was added to each well to initiate the reaction. The reaction mixture was left to stand at 37° C. for 30 min, and then 20 μl each of a reaction terminating solution [20 mM Tris-hydrochloric acid (pH 7.4), 150 mM sodium chloride, 0.1% BSA, 0.05% Tween 20 and 1 μM CGP-29287] was added to each well to terminate the reaction, thus an enzyme reaction solution was obtained. The amount of angiotensin I produced by an enzyme reaction was quantified by Enzyme Immuno Assay (EIA) described below.

Anti-angiotensin I antibody (Peninsula Laboratories Inc.) diluted 5,000-fold with PBS was added to each well of a 384 well black plate (Nalge Nunc International Co., Ltd.) by 25 μl, and left standing overnight at 4° C. to immobilize the antibody in the plate. The antibody solution was removed, PBS solution (100 μl) containing 1% BSA was added to each well, and the mixture was left standing at room temperature for 2 hr for blocking. The blocking solution was removed, and each well was washed 5 times with 100 μl of 0.05% Tween20-PBS. An angiotensin I standard solution (Wako Pure Chemical Industries, Ltd.) prepared to 0.156-10 nM with an enzyme reaction solution or buffer [20 mM tris-hydrochloric acid (pH 7.4), 150 mM sodium chloride, 0.1% BSA, 0.05% Tween20] was dispensed to each well by 10 μl. Then, a biotinated angiotensin I solution (AnaSpec, 15 μl) prepared to 1.6 nM with a buffer [20 mM tris-hydrochloric acid (pH 7.4), 150 mM sodium chloride, 0.01% BSA, 0.05% Tween20] was added to each well, mixed with a plate mixer and left standing at room temperature for 1 hr. The solutions were removed from each well, and each well was washed 5 times with 100 μl of 0.05% Tween20-PBS. Horseradish peroxydase Streptavidin (PIERCE Biotechnology inc., 25 μl) diluted to 100 ng/ml with a buffer [20 mM tris-hydrochloric acid (pH 7.4), 150 mM sodium chloride, 0.1% BSA, 0.05% Tween 20] was added to each well and the mixture was left standing at room temperature for 30 min. The solutions were removed from each well, and each well was washed 5 times with 100 μl of 0.05% Tween20-PBS. SuperSignal ELISA femto Maximum Sensitivity Substrate (PIERCE Biotechnology Inc.) was added by 25 μl and luminescence intensity was measured by EnVision (Perkin Elmer Inc.). An analytical curve was drawn from the luminescence intensity of a well containing an angiotensin I standard solution, and the amount of angiotensin I produced by an enzyme reaction was calculated and used as an index of renin activity.

While the reaction rate of the well where 100% DMSO only was added was taken as 0% inhibition rate, and the reaction rate of the well where angiotensin I was not contained was taken as 100% inhibition rate, the renin inhibitory activity of the wells where the test compound (containing 100% DMSO) was added was calculated.

### (8) Results

Example compounds 1-367, 369-429 were measured by the method of the above-mentioned (6) or (7). As a result, all compounds showed a renin inhibitory activity of 30% or above at a concentration of 1 μM.

Example compounds 430-596, 645-766 were measured by the method of the above-mentioned (7). As a result, all compounds showed a renin inhibitory activity of 25% or above at a concentration of 0.1 μM.

It is clear therefrom that compound (I) of the present invention has a superior renin inhibitory activity.

### Sequence Listing Free Text

[SEQ ID NO: 1] primer
[SEQ ID NO: 2] primer
[SEQ ID NO: 3] primer
[SEQ ID NO: 4] primer
[SEQ ID NO: 5] primer
[SEQ ID NO: 6] primer
[SEQ ID NO: 7] partial sequence of human angiotensinogen
[SEQ ID NO: 8] substrate peptide of renin

### INDUSTRIAL APPLICABILITY

Compound (I) has superior renin inhibitory activity and thus is useful as an agent for the prophylaxis or treatment of hypertension, various organ damages attributable to hypertension, and the like.

This application is based on patent application Nos. 120292/2007 and 207271/2007 filed in Japan, the contents of which are hereby incorporated by reference.

1. A compound represented by the formula: 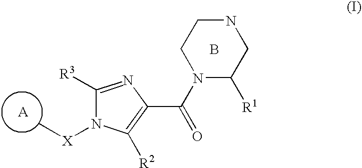
wherein
R¹ is a substituent,
R² is a cyclic group optionally having substituent(s), C₁₋₁₀ alkyl optionally having substituent(s), C₂₋₁₀ alkenyl optionally having substituent(s) or C₂₋₁₀ alkynyl optionally having substituent(s),
R³ is a hydrogen atom, a halogen atom, C₁₋₆ alkyl or C₁₋₆ alkoxy,
X is bond or spacer having 1 to 6 atoms in the main chain,
ring A is C₅₋₇ cycloalkane optionally having substituent(s), and
ring B is piperazine optionally further having substituent(s) besides R¹,
or a salt thereof.

2. The compound of claim 1, wherein R¹ is a hydrocarbon group optionally having substituent(s).

3. The compound of claim 1, wherein R² is C₆₋₁₄ aryl optionally having substituent(s) or C₃₋₁₀ cycloalkyl optionally having substituent(s).

4. The compound of claim 1, wherein R³ is a hydrogen atom, a halogen atom, C₁₋₃ alkyl or C₁₋₃ alkoxy.

5. The compound of claim 1, wherein X is bond or C₁₋₆ alkylene optionally having substituent(s).

6. The compound of claim 1, wherein ring A is C₅₋₇ cycloalkane optionally having substituent(s) selected from a halogen atom, a hydrocarbon group optionally having substituent(s), hydroxy optionally having a substituent and amino optionally having substituent(s).

7. The compound of claim 1, wherein ring B is a ring represented by the formula: 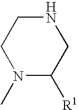
wherein R¹ is as defined in claim 1.

8. A compound represented by the formula: 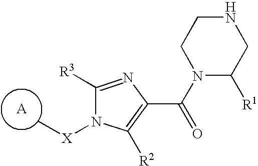
wherein
R¹ is
(a) C₁₋₆ alkyl substituted by hydroxy optionally having a substituent,
(b) C₁₋₆ alkyl substituted by phenylamino optionally having substituent(s), or
(c) C₇₋₁₃ aralkyl optionally having substituent(s);
R² is optionally halogenated C₆₋₁₀ aryl;
R³ is a hydrogen atom, a halogen atom, C₁₋₃ alkyl or C₁₋₃ alkoxy;
X is bond or C₁₋₆ alkylene optionally having substituent(s); and
ring A is
(a) C₅₋₇ cycloalkane substituted by hydroxy optionally having a substituent, and optionally further substituted by C₁₋₃ alkyl optionally having substituent(s), or
(b) C₅₋₇ cycloalkane substituted by amino optionally having substituent(s).

9. (1S,2R)-1-(Methoxymethyl)-2-{4-[((2R)-2-{2-[(2-methyl-1,3-benzothiazol-5-yl)oxy]ethyl}piperazin-1-yl)carbonyl]-5-phenyl-1H-imidazol-1-yl}cyclohexanol or a salt thereof.

10. Methyl [(1S,2S)-2-(4-{[(2R)-2-(3,5-difluorobenzyl)piperazin-1-yl]carbonyl}-5-phenyl-1H-imidazol-1-yl)cyclohexyl]carbamate or a salt thereof.

11. (1S,2R)-1-(Methoxymethyl)-2-[5-phenyl-4-({(2R)-2-[(5-phenyl-1,3,4-oxadiazol-2-yl)methyl]piperazin-1-yl}carbonyl)-1H-imidazol-1-yl]cyclohexanol or a salt thereof.

12. (1S,2R)-1-(Methoxymethyl)-2-[4-({(2R)-2-[2-(2-methoxy-4-methylphenoxy)ethyl]piperazin-1-yl}carbonyl)-5-phenyl-1H-imidazol-1-yl]cyclohexanol or a salt thereof.

13. Ethyl [(1S,2S)-2-(4-{[(2R)-2-(3,5-difluorobenzyl)piperazin-1-yl]carbonyl}-5-phenyl-1H-imidazol-1-yl)cyclohexyl]carbamate or a salt thereof.

14. (1S,2R)-2-(4-{[(2R)-2-Benzylpiperazin-1-yl]carbonyl}-5-phenyl-1H-imidazol-1-yl)-1-(methoxymethyl)cyclohexanol or a salt thereof.

15. (1S,2R)-2-[4-{[(2R)-2-Benzylpiperazin-1-yl]carbonyl}-5-(3-fluorophenyl)-1H-imidazol-1-yl]-1-(methoxymethyl)cyclohexanol or a salt thereof.

16. Methyl [(1S,2S)-2-(4-{[(2R)-2-(2-anilinoethyl)piperazin-1-yl]carbonyl}-5-phenyl-1H-imidazol-1-yl)cyclohexyl]carbamate or a salt thereof.

17. (1S,2R)-1-(Methoxymethyl)-2-(4-{[(2R)-2-(2-morpholinobenzyl)piperazin-1-yl]carbonyl}-5-phenyl-1H-imidazol-1-yl)cyclohexanol or a salt thereof.

18. (1S,2R)-2-(4-{[(2R)-2-Benzylpiperazin-1-yl]carbonyl}-5-phenyl-1H-imidazol-1-yl)-1-methylcyclohexanol or a salt thereof.

19. (1S,2R)-1-(Methoxymethyl)-2-{4-[((2R)-2-{2-[(3-methoxyphenyl)amino]ethyl}piperazin-1-yl)carbonyl]-5-phenyl-1H-imidazol-1-yl}cyclohexanol or a salt thereof.

20. (1S,2R)-1-(Methoxymethyl)-2-(5-phenyl-4-{[(2R)-2-(2-{[4-(1H-pyrazol-1-yl)phenyl]amino}ethyl)piperazin-1-yl]carbonyl}-1H-imidazol-1-yl)cyclohexanol or a salt thereof.

21. (1S,2R)-1-(Methoxymethyl)-2-{4-[((2R)-2-{2-[(5-methoxy-2-methylphenyl)amino]ethyl}piperazin-1-yl)carbonyl]-5-phenyl-1H-imidazol-1-yl}cyclohexanol or a salt thereof.

22. (1S,2R)-2-{4-[((2R)-2-{2-[(2-Ethyl-1,3-benzoxazol-5-yl)amino]ethyl}piperazin-1-yl)carbonyl]-5-phenyl-1H-imidazol-1-yl}-1-(methoxymethyl)cyclohexanol or a salt thereof.

23. 1-[(4-{[(2R)-2-(2-Anilinoethyl)piperazin-1-yl]carbonyl}-5-phenyl-1H-imidazol-1-yl)methyl]cyclohexanol or a salt thereof.

24. A prodrug of the compound of claim 1.

25. A pharmaceutical agent comprising the compound of claim 1 or a prodrug thereof.

26. The pharmaceutical agent of claim 25, which is a renin inhibitor.

27. The pharmaceutical agent of claim 25, which is an agent for the prophylaxis or treatment of hypertension.

28. The pharmaceutical agent of claim 25, which is an agent for the prophylaxis or treatment of various organ damages attributable to hypertension.

29. A method for the prophylaxis or treatment of hypertension in a mammal, which comprises administering an effective amount of the compound of claim 1 or a prodrug thereof to the mammal.

30. Use of the compound of claim 1 or a prodrug thereof for the production of an agent for the prophylaxis or treatment of hypertension.


SEQUENCE LISTING (8 sequences)

SEQ ID NO: 1 - DNA, 21 bp, Artificial
  primer
aagcttatggatggatggaga

SEQ ID NO: 2 - DNA, 21 bp, Artificial
  primer
ggatcctcagcgggccaaggc

SEQ ID NO: 3 - DNA, 30 bp, Artificial
  primer
aagcttatgcggaagcgagcaccccagtct

SEQ ID NO: 4 - DNA, 59 bp, Artificial
  primer
ggatcctcacttgtcatcgtcgtccttgtagtctgctgtgctcagcgggttggccacgc

SEQ ID NO: 5 - DNA, 39 bp, Artificial
  primer
ccttaagcttccaccatgcggaagcgagcaccccagtct

SEQ ID NO: 6 - DNA, 39 bp, Artificial
  primer
ttggatcctcatgctgtgctcagcgggttggccacgcgg

SEQ ID NO: 7 - protein, 15 aa, Artificial
  partial sequence of human angiotensinogen
DRVYIHPFHLVIHNE

SEQ ID NO: 8 - protein, 16 aa, Artificial
  substrate peptide for renin
XDRVYIHPFHLVIHQR